(19) United States
(12) Patent Application Publication
(10) Pub. No.: US 2020/0308627 A1
(43) Pub. Date: Oct. 1, 2020

(54) UNIVERSAL METHOD FOR EXTRACTING NUCLEIC ACID MOLECULES FROM A DIVERSE POPULATION OF ONE OR MORE TYPES OF MICROBES IN A SAMPLE

(71) Applicant: Sun Genomics, Inc., San Diego, CA (US)

(72) Inventor: Suneer Jain, San Diego, CA (US)

(21) Appl. No.: 16/664,771

(22) Filed: Oct. 25, 2019

Related U.S. Application Data
(63) Continuation-in-part of application No. 16/373,387, filed on Apr. 2, 2019.
Application No. 16/373,387 is a continuation-in-part of application No. 16/042,831, filed on Jul. 23, 2018.
Application No. 16/042,831 is a division of application No. 15/824,754, filed on Nov. 28, 2017, now Pat. No. 10,428,370.
Application No. 15/824,754 is a continuation of application No. PCT/US2017/051849, filed on Sep. 15, 2017.
(60) Provisional application No. 62/751,484, filed on Oct. 26, 2018.
(60) Provisional application No. 62/412,787, filed on Oct. 25, 2016.
(60) Provisional application No. 62/395,316, filed on Sep. 15, 2016.
(60) Provisional application No. 62/651,620, filed on Apr. 2, 2018.

(51) Int. Cl. C12Q 1/6806; C12Q 1/686; C12Q 1/6827; A61K 35/741; G16B 20/00

(52) U.S. Cl. CPC C12Q 1/6806; C12Q 1/686; A61K 2035/115; A61K 35/741; G16B 20/00; C12Q 1/6827

(57) ABSTRACT

Disclosed herein are methods of extracting genetic material from a diverse population of one or more types of microbes in a sample. Microbes can be prokaryotes or eukaryotes and may include bacteria, archaea, fungi, protozoa, helminths, parasites, viruses, phages, and others. Extraction may be from a single sample and subsequent identification may be through a molecular method such as qPCR, PCR, RFLP, SSCP, allele specific PCR, targeted sequencing, pull down sequencing, whole shotgun sequencing, or other methods. Also provided are methods that include extracting nucleic acid molecules from a variety of organisms such as fungi (i.e., Saccharomyces spp.), animal cells (Bos taurus), plants (e.g., Hordeum vulgare) from the gut of a human subject, performing a metagenomics analysis therefrom, and determining a probiotic treatment or dietary guidance for the subject based on the metagenomics analysis.

### CROSS REFERENCE TO RELATED APPLICATIONS

This application is a continuation-in-part application of U.S. application Ser. No. 16/373,387 filed on Apr. 2, 2019, now pending, which is a continuation-in-part application of U.S. application Ser. No. 16/042,831 filed on Jul. 23, 2018, now pending, which is a divisional application of U.S. application Ser. No. 15/824,754 filed Nov. 28, 2017, which is a continuation application of International Application No. PCT/US2017/051849 filed Sep. 15, 2017, now pending, which claims the benefit under 35 U.S.C. § 119(e) to U.S. Application Ser. No. 62/412,787 filed Oct. 25, 2016 and to U.S. Application Ser. No. 62/395,316 filed Sep. 15, 2016, both now expired; and this application claims the benefit under 35 U.S.C. § 119(e) to U.S. Application Ser. No. 62/651,620 filed Apr. 2, 2018 and to U.S. Application Ser. No. 62/751,484 filed Oct. 26, 2018. The disclosure of each of the prior applications is considered part of and is incorporated by reference in the disclosure of this application.

### BACKGROUND OF THE INVENTION

### Field of Invention

The present invention relates generally to genomic analysis and more particularly to a method of extracting and analyzing nucleic acid molecules associated with food from a diverse population of microbes in a biological sample.

### Background Information

About 100 trillion microorganisms live in and on the human body vastly outnumbering the body's approximately 10 trillion human cells. These normally harmless viruses, bacteria and fungi are referred to as commensal or mutualistic organisms. Commensal and mutualistic organisms help keep our bodies healthy in many ways. Together all of the microorganisms living in and on the body—commensal, mutualistic and pathogenic—are referred to as the microbiome and their equilibrium and associated metabolome is closely linked to an individual's health status and vice-versa.

Advances in nucleic acid sequencing has created an opportunity to quickly and accurately identify and profile the microbiome inhabiting the gut and subcutaneous tissue. The optimal flora also interacts with the host immune system in a synergistic way further propagating its health benefits. The associated metabolome of individuals can also be profiled either by a mass-spectrometry based system or using genomics-based metabolome modeling and flux-balance analysis and used to make a healthy metabolome profile. All these methodologies can be used to dissect the complexity of microbial communities.

### SUMMARY OF THE INVENTION

The present invention is directed to a method of extracting nucleic acid molecules from a diverse population of microbes in a biological, environmental, dietary supplement, or other ecological microbial organism heterogeneous populations sample and use of nucleic acid or extracts through processing steps and analysis for the determination of probiotic customization to an individual. Processing steps specific to this invention include, RNA or DNA clean-up, fragmentation, separation, or digestion; library or nucleic acid preparation for downstream applications, such as PCR, qPCR, digital PCR, or sequencing; preprocessing for bioinformatic QC, filtering, alignment, or data segregation; metagenomics or human genomic bioinformatics pipeline for microbial species taxonomic assignment; and other organism alignment, identification, and variant interpretation.

The present invention also describes a universal method for using samples for DNA extraction and determination of food consumption based on food DNA sequence from a database of meats, plants, fruits, vegetables, and/or microbes contained with these organisms. Disclosed herein are methods of extracting genetic material from a diverse population of one or more types of cells or cell components in a sample and determining the consumed food and nutritional breakdown for the improvement of health and prevention of disease.

Accordingly, in one aspect, the invention provides a method for preparing a sample for analysis. The method includes: a) mixing the sample with a first lysis solution comprising a detergent, e.g., SDS, and a chelator, e.g., EDTA; b) adding a second lysis solution having a lysozyme to the mixture of step a); and c) adding a third lysis solution comprising a chaotropic agent, e.g., urea, lithium acetate, guanidine hydrochloride, and the like, to the mixture of step b). Pre-processing steps may include physical lysis may be used to further optimize nucleic acid yield. Examples of mechanical lysis include sonication, bead mixing, and bead mill homogenization.

In a similar aspect, the method includes: a) mixing a sample, such as a stool sample, with a liquid nitrogen solution: b) adding a first lysis solution, the first lysis solution comprising a detergent and a chelator, e.g., SDS, and a chelator, e.g., EDTA; and c) adding a second lysis solution, the second lysis solution including a chaotropic agent, e.g., urea, lithium acetate, guanidine hydrochloride. Pre-processing steps may include physical lysis may be used to further optimize nucleic acid yield. Examples of mechanical lysis include sonication, bead mixing, and bead mill homogenization.

In another aspect, the invention provides a method of determining food consumption of a subject. The method includes: a) extracting genetic material from a stool sample obtained from the subject, said genetic material extracted according to a method of the disclosure; and b) subjecting the genetic material extracted from the first sample to metagenomics analysis to determine the food consumption of the subject. In embodiments, the method further includes treating the subject with a probiotic or a food stuff based on the analysis of food consumption.

In another aspect, the invention provides a method of monitoring a probiotic treatment of a subject. The method includes: a) extracting genetic material from any microbes present in a first sample obtained from the subject, said genetic material extracted according to a method of the disclosure; b) subjecting the genetic material extracted from the first sample to metagenomics analysis; c) treating the subject with a probiotic and then extracting genetic material from any microbes present in a second sample obtained from the subject in the same manner as the extraction of genetic material from the first sample; d) performing metagenomics analysis on the extracted genetic material from the second sample; and e) comparing the results of the metagenomics analysis of the first sample with the metagenomics analysis of the second sample.

In yet another aspect, the invention provides a method comprising calculating a probiotic score from probiotic organisms detected in a gut with or without additional chemistry or genetic tests.

In still another aspect, the invention provides a method comprising calculating a score for a microbiome, the score being used to assess if the microbiome is in dysbiosis, neutral, or stable.

The invention further provides a computing system comprising: a memory; and one or more processors coupled to the memory, the one or more processors configured to perform operations to perform a method of the present invention.

The invention also provides an automated platform for performing a method of the invention.

The invention provides an all-in-one method for extracting nucleic acids from a diverse flora of microbes from a biological, environmental, dietary supplement, or other ecological microbial organism heterogeneous populations sample.

In embodiments, the invention may be used in determining composition and relative abundance of microbes, via analyzing their respective nucleic acids, in probiotics and environmental samples. DNA is purified and used downstream for nucleic acid analysis (notably metagenomics analysis where genome of more than one species/subspecies is identified).

In yet another aspect, the invention provides a method of detection, diagnosis and/or treatment for reduction or elimination of opportunistic pathogens or disorder causing microbes of the gut using probiotics, pre-biotics or metabolites of the gut microbiome.

In still another aspect, the invention provides use of strains together, in any combination, or singly listed in Tables 5-14 to reduce the abundance of disease or disorder causing microbes.

In another aspect, the invention provides a method which includes: a) assaying the expression level of one or more gastrointestinal target sequences from a sample from a subject; and b) administering a probiotic composition to the subject.

In another aspect, the invention provides system including: a) a probe set comprising a plurality of polynucleotides that hybridize to at least a portion of one or more gastrointestinal target sequences; and b) a computer readable medium encoding a computer model or algorithm for analyzing an expression level and/or expression profile of the target sequences hybridized to the probe in a sample from a subject.

In still another aspect, the invention provides a method of treating gastrointestinal dysbiosis including: a) assaying the expression level of one or more gastrointestinal target sequences from a sample from a subject; and b) administering a probiotic composition to the subject.

In yet another aspect, the invention provides a composition of probiotics, wherein the composition is determined by assaying the expression level of one or more gastrointestinal target sequences from a sample from a subject and wherein the probiotics correct gastrointestinal dysbiosis in the subject.

Both the foregoing general description and the following detailed description are exemplary and explanatory only and are intended to provide further explanation of the invention as claimed. Any accompanying drawings are included to provide a further understanding of the invention and are incorporated in and constitute part of this specification, illustrate several embodiments of the invention, and together with the description serve to explain the principles of the invention.

### BRIEF DESCRIPTION OF THE DRAWINGS

FIG. 1 shows the most abundant microbes identified in a patient stool sample.

FIGS. 2A-2D show the species of bacteria, viruses, archaea eukaryotic organisms identified in a patient sample. 2A shows all of the organisms identified in the sample. 2B shows the species of viruses identified in the sample. 2C shows the archaea species identified in the sample. 2D shows the species of eukaryotic organisms identified in the sample.

FIGS. 3A-3D show the species of bacteria, viruses, archaea eukaryotic organisms identified in a patient sample. 3A shows all of the organisms identified in the sample. 3B shows the species of viruses identified in the sample. 3C shows the archaea species identified in the sample. 3D shows the species of eukaryotic organisms identified in the sample.

FIGS. 4A-4D show the species of bacteria, viruses, archaea eukaryotic organisms identified in a patient sample. 4A shows all of the organisms identified in the sample. 4B shows the species of viruses identified in the sample. 4C shows the archaea species identified in the sample. 4D shows the species of eukaryotic organisms identified in the sample.

FIG. 5 shows the least abundant microbes identified in a patient stool sample.

FIG. 6 shows the probiotics identified in a patient sample.

FIG. 7 shows the probiotics identified in a patient sample.

FIG. 8 shows a comparison of the relative abundance of microbes identified in a subject sample with the relative abundance of the microbes in the general population.

FIG. 9 is a chart listing the microbes which appear in samples from the subject's sample with the highest and lowest frequency.

FIG. 10 shows the breakdown of unique species of archaea, bacteria, fungi, protozoa and viruses found in the microbiome of the subject's sample.

FIGS. 11A-11C show the species of Mollusca, Bovidae and Liliopsida organisms identified in a patient sample. 11A Mollusca. 11B Bovidae. 11C Liliopsida.

FIG. 12 shows the microbiome analysis of a subject having latent Hepatitis B diagnosed using the disclosed methods.

FIG. 13 shows the opportunistic pathogen content of a subject's sample before and after drug intervention against small intestinal overgrowth (SIBO).

FIGS. 14A-14C are examples of microbiome profiles used to create the healthy reference profile.

FIGS. 15A-15B show the probiotics profile and microbe profile of a subject before antibiotic treatment, after antibiotic treatment and after probiotic treatment. 15A probiotic profile. 15B Microbe profile.

FIGS. 16A-16E show the microbiome profile of a subject. 16A is the probiotic profile. 16B is a list of the top 10 microbes. 16C is a chart of other significant gut influencers. 16D is a comparison of the genus and families of interest compared to a healthy reference. 16E is a summary of the key microbes detected.

FIGS. 17A-17E show the microbiome profile of a subject. 17A is a summary of the key microbes detected. 17B is a list of the top 10 microbes. 17C is a chart of other significant gut influencers. 17D is a comparison of the genus and families of interest compared to a healthy reference. 17E is the probiotic profile.

FIGS. 18A-18B show the analysis of the microbiome for subject SG00095. 18A shows the top 10 microbes identified. 18B shows a comparison of the microbes with a healthy reference.

FIGS. 19A-19B show the analysis of the microbiome for subject SG00443. 19A shows the top 10 microbes identified. 19B shows a comparison of the microbes with a healthy reference.

FIGS. 20A-20B show the analysis of the microbiome for subject SG00216. 20A shows the top 10 microbes identified. 20B shows a comparison of the microbes with a healthy reference.

FIGS. 21A-21B show the analysis of the microbiome for subject SG00346. 21A shows the top 10 microbes identified. 21B shows a comparison of the microbes with a healthy reference.

FIGS. 22A-22B show the analysis of the microbiome for subject SG00279. 22A shows the top 10 microbes identified. 22B shows a comparison of the microbes with a healthy reference.

FIGS. 23A-23B show the analysis of the microbiome for subject SG00210. 23A shows the top 10 microbes identified. 23B shows a comparison of the microbes with a healthy reference.

### DETAILED DESCRIPTION OF THE INVENTION

The present invention provides a universal method for extracting nucleic acid molecules from a diverse population of one or more types of microbes in a sample. The types of microbes include: gram-positive bacteria, gram-positive bacterial spores, gram-negative bacteria, archaea, protozoa, helminths, algae, fungi, fungal spores, viruses, viroids, bacteriophages, and rotifers. In some embodiments, the diverse population is a plurality of different microbes of the same type, e.g., gram-positive bacteria. In some embodiments, the diverse population is a plurality of different types of microbes, e.g., bacteria (gram-positive bacteria, gram-positive bacterial spores and/or gram-negative), fungi, viruses, and bacteriophages.

Because different types of microbes have different compositions and mechanisms to protect their own genetic material it is often difficult to extract the genetic material from one type of microbe without compromising the ability to also extract the genetic material of another type of microbe in the same biological sample. The present invention, however, allows the extraction of genetic material from different types of microbes in a sample without sacrificing the amount of genetic material that can be obtained from one type of microbe by extracting the genetic material of another type of microbe in the same sample. According to the present invention, the sample comprising the microbes may be a biological sample, environmental sample, an artificially created sample (e.g., a laboratory test or control sample, a sample of a probiotic composition or supplement, etc.), or the like. Examples of biological samples include tissue samples, blood samples, plasma samples, cerebrospinal fluid samples, urine samples, fecal samples, samples of material obtained from the digestive tract, biological secretions (e.g., semen, vaginal secretions, breast milk, tears, saliva, etc.), and the like. Solid samples may be liquefied or mixed with a solution, and then genetic material of the microbes present in the liquefied sample, mixture, or solution obtained from the mixture may be extracted in accordance with the present invention. The extracted genetic material may be subjected to further processing and analysis such as purification, amplification, and sequencing.

In some embodiments, the extracted genetic material is subjected to metagenomics analysis to, for example, identify the one or more types of microbes in the sample from which the genetic material was extracted. In additional embodiments, full whole genome shotgun sequencing can be performed on prepared extracted nucleic acid material from human fecal samples. Preparations include nucleic acid clean up reactions to remove organic solvents, impurities, salts, phenols, and other process inhibiting contaminants. Additional preparations include nucleic acid library prep from each sample where the gDNA is subject to modifications and/or amplifications to prep the sample for sequencing on a sequencing platform such as massively parallel sequencing by synthesis, nanopore, long read, and/or CMOS electronic, sequencing methods.

As disclosed herein, the inventive method allows the successful extraction of genetic material from one or more different types of microbes present in the same sample by subjecting the microbes to three different compositions in a particular order. The method according to the present invention comprises first lysing any gram-negative bacteria present in the sample, which is followed by digesting the polysaccharide component of the cell walls of any yeast and bacteria present in the sample, and then disrupting any cell walls that are intact after the second step with a chaotropic agent.

Briefly, in an embodiment, the first step comprises mixing the sample with a first lysis solution comprising a detergent (e.g., sodium dodecyl sulfate (SDS)) and a chelator (e.g., ethylenediaminetetraacetic acid (EDTA)) to lyse any gram-negative bacteria present in the sample. The first lysis solution may further include one or more buffers (e.g., Tris), one or more mild detergents (e.g., Triton™ X-100), and/or one or more proteases (e.g., proteinase K).

After the first step, the sample is mixed with a second lysis solution comprising a lysozyme to digest the polysaccharide component of any yeast and bacterial cell walls present in the mixture. Because lysozyme may inhibit the activity of the first lysis solution, it is important that contact of the sample with the second lysis solution occurs after treating the sample with the first lysis solution.

After treatment with the second lysis solution, a third lysis solution comprising a chaotropic agent (e.g., urea, lithium acetate, guanidine hydrochloride, and the like) is added to the mixture to disrupt any cell walls that are not digested by the second lysis solution. The third lysis solution may include a detergent such as SDS.

In some embodiments, both the first lysis solution and the third lysis solution comprise SDS at a working concentration of between 1-10% w/v. In some embodiments, after treatment with the third lysis solution, the mixture is further treated with a fourth lysis solution comprising a chaotropic agent (e.g., urea, lithium acetate, guanidine hydrochloride, and the like) and Proteinase K. In some embodiments where the chaotropic agent of the third lysis solution is lithium acetate, the mixture is then subjected to heat shock treatment and may then be treated with the fourth lysis solution.

In certain aspects, the following disclosure describes a universal method for using stool samples for DNA extraction and determination of food consumption based on food DNA sequence from a database of meats, plants, fruits, vegetables, and/or microbes contained with these organisms. Disclosed herein are methods of extracting genetic material from a diverse population of one or more types of cells or cell components in a sample and determining the consumed food and nutritional breakdown for the improvement of health and prevention of disease.

In some embodiments, biological secretions (e.g., semen, vaginal secretions, breast milk, tears, saliva, blood, urine, and the like) are obtained from the digestive tract, and the like. Solid samples may be liquefied or mixed with a solution, and then genetic material of any food item containing genetic material, such as plant based (seedlings, leaves, cotyledons, seeds, endosperm, tissue culture callus, roots, and the like), animal based, fungi based, or protista based foods in the liquefied sample, mixture, or solution obtained from the mixture may be extracted in accordance with the present invention or other standard nucleic acid extraction protocols known in the art. In some embodiments, the extracted genetic material may be subjected to further processing and analysis, such as purification, amplification, and sequencing. In some embodiments, the extracted genetic material is subjected to metagenomics analysis to, for example, identify the one or more types of organisms in the sample from which the genetic material was extracted.

In some embodiments the database that the metagenomic analysis will utilize has been customized for a specific purpose of identifying and taxonomically assigning, within the appropriate phylogeny, the nucleic acids with relative abundances of organisms or components of organisms ingested by humans or other animals. In some embodiments and additional data table or database may be used as a lookup of the relative abundances of organisms to determine macronutrient content of an organism's gut sample as a representation of their diet. In some embodiments this macronutrient breakdown may include fats, carbohydrates, proteins, vitamins minerals, and subcomponents of any macronutrients.

As disclosed herein, the inventive method allows the successful extraction of genetic material from one or more different types of organisms, one or more of an organism's cells, or cellular matrices or organelles present in the same sample by subjecting the sample to isolation, purification, or other methods for capturing nucleic acids. The method according to the present invention comprises lysing or disrupting any food cells in the sample, including but not limited to any cell walls and cell membranes, digesting the polysaccharide or lignin component of any cell walls or membranes of any fungi, plant, mammalian, or protista cells present in the sample, and disrupting any cell walls that are intact after the digestion step with a chaotropic agent.

The present invention includes a step to physically disrupt the cell wall or membranes of food cells by liquid nitrogen flash freezing and immediate mechanical disruption or grinding to break down cell walls and keep harmful cell enzyme inactivated prior to chemical lysis. The present invention includes a step comprising mixing the sample with a first lysis solution comprising a detergent (e.g., sodium dodecyl sulfate (SDS)) and a chelator (e.g., ethylenediaminetetraacetic acid (EDTA)) to lyse any animal cells present in the sample. The first lysis solution may further include one or more buffers (e.g., Tris), one or more mild detergents (e.g., Triton™ X-100, Cetyltrimethylammonium bromide), and/or one or more proteases (e.g., proteinase K). In particular embodiments, the first lysis solution comprises SDS at a working concentration 1-10% w/v. The present invention includes a step comprising mixing the sample with a second lysis solution comprising a chaotropic agent (e.g., urea, lithium acetate, guanidine hydrochloride, and the like). The second lysis solution may include a detergent, such as SDS. In particular example embodiments, the first and second lysis solutions may be added in any particular order.

In some embodiments, the present invention may include a step comprising mixing the sample with a third lysis solution comprising a lysozyme to digest the polysaccharide component of any fungi or bacteria cell walls present in the mixture. In some embodiments, the mixture may be further treated with a fourth lysis solution comprising a chaotropic agent (e.g., urea, lithium acetate, guanidine hydrochloride, and the like) and Proteinase K. In some embodiments where the chaotropic agent of the fourth lysis solution is lithium acetate, the mixture may then be subjected to heat shock treatment and may then be treated with the fourth lysis solution. In particular example embodiments, the third and/or fourth solution may be added to the mixture at any point to disrupt any cell walls that are not digested by any previous lysis solution.

In some embodiments, if the sample has or is suspected of having bacterial and/or fungal spores, the sample may be subjected to a pretreatment step that induces germination of the cell walls of the spores before contact with the first lysis solution. The pretreatment step may comprise mixing the sample with a chemical such as a mild detergent, e.g., Tween-80, to induce germination or cultivating the sample under conditions (e.g., temperature) that induce germination. In some embodiments, where germination is induced with a chemical, the chemical is preferably one that does not inhibit, reduce, or modify the activity or effectiveness of the first, second, and third lysis solutions.

In some embodiments, the method according to the present invention may further include one or more mechanical treatment steps that cause physical lysis by mechanical methods including sonication, bead mixing, bead mill homogenization, pressurization, microfluidization, and the like. In some embodiments, a mechanical treatment step is performed before subjecting the sample to the first lysis solution.

In embodiments, the method according to the present invention is capable of extracting nucleic acid molecules from a variety of microbes including yeast (i.e., Saccharomyces spp.), gram-negative bacteria (e.g., Acinetobacter spp.), gram-positive bacteria (e.g., Bifidobacterium spp.), viruses (e.g., Sclerotinia spp.), spores (Bacillus spp.) Helminths (tapeworm Echinococcus spp.), Protozoa (Sarcodina—the ameba, e.g., Entamoeba) and phages (e.g., Lactobacillus phages).

In embodiments, the method according to the present invention is capable of extracting nucleic acid molecules from a variety of organisms including fungi (i.e., Saccharomyces spp.), animal cells (Bos taurus), plants (e.g., Hordeum vulgare).

The following examples are intended to illustrate but not to limit the invention.

### Extraction Method A

A range of 10 mg to 5000 mg of sample were added to a sterile 2 milliliters (mL) micro centrifuge tube. Bead beating may optionally be performed by adding 400 microliters (μL) of bead pure mixture and vortexing for about 30 seconds at 8000 rpm. If, however, high-molecular weight nucleic acids, e.g., genomic DNA, are desired to be obtained, bead beating is preferably avoided.

### First Lysis Solution Treatment Step

To lyse any gram-negative bacteria in the sample, the sample was subjected to a First Lysis Solution by adding about 400 μL of Digestion Buffer (1% w/v SDS, 25 mM Tris HCl, 2.5 mM EDTA, 1% Triton™ X-100, pH 8) and about 20 μl of Proteinase K to the sample and gently mixed. The mixture was then incubated for about 30 minutes at 55° C.

### Second Lysis Solution Treatment Step

To lyse any gram-positive bacteria in the sample, a Second Lysis Solution comprising a glucoside hydrolase (“lysozyme”) was added to the mixture obtained from the First Lysis Solution Treatment Step to give a final lysozyme concentration of 1 mg/mL and a pH of about 8.0. Suitable glucoside hydrolases may be obtained from a variety of sources including egg whites, tears, or mucus or saliva of various animals. The mixture was then incubated for a period of about 1 to 24 hours at 37° C.

### Third Lysis Solution Treatment Step

To lyse any fungal and/or yeast cells present in the sample, a Third Lysis Solution comprising 1M lithium acetate in distilled sterile H₂O and 5% w/v SDS was added to obtain about a 1:5 dilution of the mixture resulting from the Second Lysis Solution Treatment Step. The treated mixture was incubated for 15 minutes at 70° C. followed by heat shock at 95° C. for one minute and then brought to room temperature by placing in a 22° C. water bath.

As the Second and Third Lysis Solution Treatment Steps are sufficient to lyse the outer coats of bacteriophages and viruses, no additional step is needed for extracting the genetic material from bacteriophages and viruses that may be present in the sample.

### Extraction Method B

### Pre-Lysis Treatment Step

100-200 mg of sample were added to a sterile 2 milliliters (mL) micro centrifuge tube. Add 500 mL of liquid nitrogen and allow sample to freeze for 30 sec. Then using a pellet pestle or saw-tooth generator probe, grind the sample thoroughly before continuing to the next step.

### First Lysis Solution Treatment Step

To lyse any animal, fungi, and protista food cell membranes in the sample, the sample was subjected to a First Lysis Solution by adding about 400 μL of Digestion Buffer (1% w/v SDS, 25 mM Tris HCl, 2.5 mM EDTA, 1% Triton™ X-100, 1.2M NaCl pH 8) and about 20 μL of Proteinase K to the sample and gently mixed. The mixture was then incubated for about 30 minutes at 55° C.

### Second Lysis Solution Treatment Step

To lyse any fungal and/or yeast cells present in the sample, a second Lysis Solution comprising 1M lithium acetate in distilled sterile H₂O and 5% w/v SDS was added to obtain about a 1:5 dilution of the mixture resulting from the first lysis solution treatment step. The treated mixture was incubated for 15 minutes at 70° C. followed by heat shock at 95° C. for one minute and then brought to room temperature by placing in a 22° C. water bath.

### Nucleic Acid Purification

In an embodiment, the genetic material extracted from the lysed microbes, i.e., the nucleic acid molecules present in the mixture after being subjected to the First, Second, and Third Lysis Solution Treatment Steps were then purified to DNA and RNA purification by splitting the mixture into two microcentifuge tubes. DNA was extracted from one tube by adding about 20 μL RNAse A and incubating for 5 minutes at room temperature. The mixture was run through a biopolymer tissue homogenizer column. If bead beating was previously performed, subjecting the mixture to the tissue homogenizer column is preferably avoided.

The eluate was then centrifuged at 1000 g for 5 minutes. The supernatant was treated with about 400 μL of DNA Lysis Solution (Guanidine HCl, Tris-EDTA, and 70% EtOH) and about 20 μL of Proteinase K, mixed, and then incubated at 55° C. for 10 minutes. Then EtOH at −22° C. was added and the mixture was mixed by inverting. The mixture may be subjected to one or more additional DNA extraction and purification methods known in the art.

RNA was extracted from the second microcentrifuge tube by running the mixture through a biopolymer tissue homogenizer column. Again, if bead beating was previously performed, subjecting the mixture to the tissue homogenizer column is preferably avoided. The eluate was then centrifuged at 1000 g for 5 minutes. The supernatant was treated with about 40 μL DNase I (1 U) in a solution of 25 mM MgCl2 and then incubated at 37° for about 15 minutes. Then the mixture was subjected to acid guanidinium thiocyanate-phenol-chloroform extraction. The mixture may be subjected to one or more additional RNA extraction and purification methods known in the art.

In an embodiment, the genetic material extracted from the lysed microbes, i.e., the nucleic acid molecules present in the mixture after being subjected to the First, Second, and pre lysis Treatment Steps were then purified to DNA and RNA purification by splitting the mixture into two microcentifuge tubes. DNA was extracted from one tube by adding about 20 ?L RNAse A and incubating for 5 minutes at room temperature.

The eluent was then centrifuged at 1000 g for 5 minutes. The supernatant was treated with about 400 μL of DNA Lysis Solution (Guanidine HCl, Tris-EDTA, and 70% EtOH) and about 20 μL of Proteinase K, mixed, and then incubated at 55° C. for 10 minutes. Then EtOH at −22° C. was added and the mixture was mixed by inverting. The mixture may be subjected to one or more additional DNA extraction and purification methods known in the art.

RNA was extracted from the second microcentrifuge tube. The eluent was then centrifuged at 1000 g for 5 minutes. The supernatant was treated with about 40 μL DNase I (1 U) in a solution of 25 mM MgCl2 and then incubated at 37° for about 15 minutes. Then the mixture was subjected to acid guanidinium thiocyanate-phenol-chloroform extraction. The mixture may be subjected to one or more additional RNA extraction and purification methods known in the art.

In some embodiments, where the quantitative expression of RNA molecules is desired, the use of an RNA stabilization buffer and bead beating is preferred to ensure release and limited degradation of RNA nucleic acid molecules.

In some embodiments where extraction of high molecular weight nucleic acid molecules is desired, bead beating and tissue homogenization column are avoided and phenol-chloroform-alcohol extraction is performed instead of silica column based extraction. In some embodiments a magnetic bead based nucleic acid purification may be performed. To remove selective molecular weights of nucleic acids and purify the sample, an agarose gel based purification and enrichment may be performed.

### Metagenomics Analysis

In an embodiment, the extracted and purified genetic material was prepared for sequencing using Illumina index adaptors and checked for sizing and quantity. Low cycle PCR was performed between 1-20 cycles for any input less then 50 ng of DNA, otherwise PCR-Free methods of library prep can be utilized for 50 ng of nucleic acid or greater. Gel purification was performed using the Qiagen Gel Purification Kit™ (Qiagen, Frederick, Md.). Clean PCR products were quantified using the Qubit™ 2.0 Fluorometer (Life Technologies, Carlsbad, Calif.). Samples were combined in equimolar amounts. Library pools were size verified using the Fragment Analyzer™ CE (Advanced Analytical Technologies Inc., Ames Iowa) and quantified using the Qubit™ High Sensitivity dsDNA kit (Life Technologies, Carlsbad, Calif.). After dilution, a 1% to 10% spike of PhiX™ V3 library control (Illumina, San Diego Calif.), pools were denatured for 5 minutes in an equal volume of 0.1 N NaOH then further diluted in Illumina's HT1 buffer. The denatured and PhiX™-spiked pool was loaded on an Illumina Next Generation™ Sequencer with Illumina sequencing primers and set for between 50-550 base, paired-end or single reads.

A range from 1000 or greater reads of sequencing for short insert methods can be used for this method. Large insert methods such as Pac Bio™, Nanopore™, or other next gene sequencing methods can use <1000 sequencing reads. Bioinformatics quality filtering was performed before taxonomy assignment. Quality trimming of raw sequencing files may include removal of sequencing adaptors or indexes; trimming 3′ or 5′ end of reads based on quality scores (Q20>), basepairs of end, or signal intensity; removal of reads based on quality scores, GC content, or non-aligned basepairs; removal of overlapping reads at set number of base pairs. Alignment of processed sequencing files was done using a custom microbial genome database consisting of sequences from Refseg™, Greengeens™, HMP™, NCBI™, PATRIC™, or other public/private data repositories or in-house data sets. This database may be used as full genome alignment scaffold, k-mer fragment alignment, or other schemes practiced in the art of metagenomics and bioinformatics. Based off the number of sequencing reads/fragments that match the database genomes we assign a taxonomic identity that is common or unique to the organism. This identifier can be a barcode, nucleotide sequence, or some other computational tag that will associate the matching sequencing read to an organism or strain within a taxonomic group. Some identifiers will be of higher order and would identify domain, kingdom, phylum, class, order, family, or genus of the organism.

The present invention is able to identify the organism at the lowest order of strain within a species.

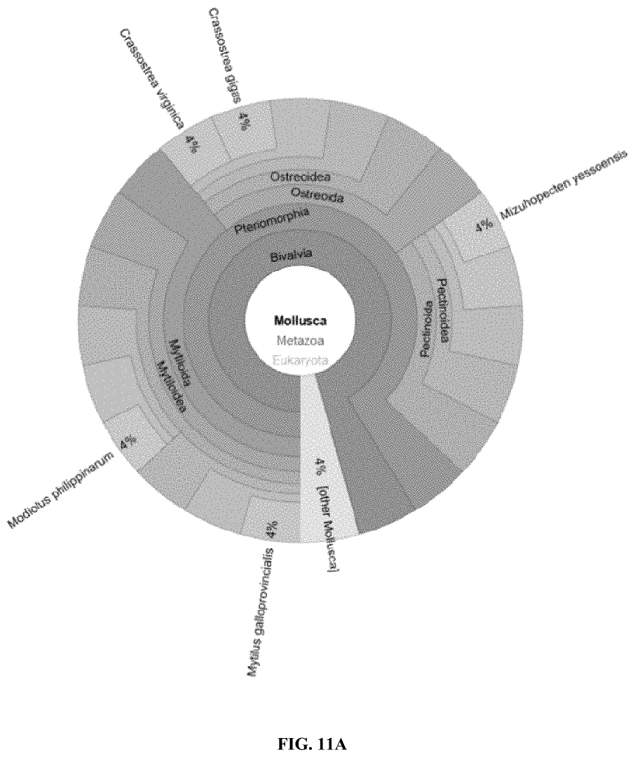

In embodiments the invention includes identification and/or analysis of one or more bacteria contained within our database (FIG. 10). Some selected examples are Bacillus clausii, Bifidobacterium animalis, Pediococcus acidilactici, Acinetobacter indicus, Lactobacillus salivarius, Acinetobacter, Bacillus amyloliquefaciens, Lactobacillus helveticus, Bacillus subtilis, Lactobacillus plantarum, Bifidobacterium longum subsp infantis, Enterococcus hirae, Lactobacillus delbrueckii subsp bulgaricus, Enterococcus, Lactobacillus rhamnosus, Lactococcus lactis, Pseudomonas stutzeri, Lactobacillus acidophilus, Klebsiella and Enterobacter cloacae strain.

In embodiments the invention includes identification and/or analysis of one or more yeast contained within our database (FIG. 10). Some selected examples are Saccharomyces sp. Boulardii, Saccharomyces kudriavzevii, Saccharomyces pastorianus and Saccharomyces cerevisiae.

In embodiments the invention includes identification and/or analysis of one or more phage or viruses contained within our database (FIG. 10). Some selected examples are Bacillus phage phi29, Enterobacteria phage HK022, Lactobacillus phage A2, Escherichia phage HK639, Phage cdtl, Sclerotinia sclerotiorum partitivirus S segment 2, Burkholderia phage BcepMu, Lactococcus prophage bIL311, Enterococcus phage phiFL4A and Streptococcus phage SM1.

Future database improvements will increase or refine the organisms that can be detected by this method.

In an embodiment, the extracted and purified genetic material was prepared for sequencing using Illumina index adaptors and checked for sizing and quantity. Low cycle PCR may be performed or standard PCR-free methods. Gel purification was performed using the Qiagen Gel Purification Kit™ (Qiagen, Frederick, Md.). Clean PCR products were quantified using the Qubit™ 2.0 Fluorometer (Life Technologies, Carlsbad, Calif.). Samples were combined in equimolar amounts. Library pools were size verified using the Fragment Analyzer™ CE (Advanced Analytical Technologies Inc., Ames Iowa) and quantified using the Qubit™ High Sensitivity dsDNA kit (Life Technologies, Carlsbad, Calif.). After dilution, a 10% spike of PhiX™ V3 library control (Illumina, San Diego Calif.), pools were denatured for 5 minutes in an equal volume of 0.1 N NaOH then further diluted in Illumina's HT1 buffer. The denatured and PhiX™-spiked pool was loaded on an Illumina™ Next Generation Sequencer with Illumina sequencing primers and set for 150 base, paired-end reads. Bioinformatics quality filtering was performed before taxonomy assignment.

Using Table 1, we determine that the individual has consumed the following:

TABLE 1

| Taxon | Reference | Species | | | | |
|---|---|---|---|---|---|---|
| Level | ID | Name | reads_subtree | reads_subtree_reestimated | abundances_subtree | abundances_subtree_reestimated |
| S | 4513 | Hordeum vulgare | 684758 | 651478 | 4.56 | 77.33 |
| S | 9858 | Capreolus capreolus | 1522 | 1522 | 0.09 | 11.10 |
| S | 3276 | Raphanus sativus | 179566 | 179566 | 10.21 | 36.03 |
| S | 161934 | Beta vulgaris | 3251 | 0 | 0.17 | 11.21 |
| S | 4565 | Triticum aestivum | 32243 | 32243 | 1.24 | 32.47 |
| S | 93759 | Corchorus olitorius | 23293 | 23293 | 0.15 | 18.56 |
| S | 9796 | Equus caballus | 69875 | 69875 | 0.43 | 11.49 |
| S | 4120 | Ipomoea batatas | 5374 | 5374 | 0.13 | 11.71 |

### Monitoring Macronutrient Intake and Dietary Guidance

In some embodiments, the present invention may be used to monitor food intake nutrition, quantity, and quality in subjects. For example, prior to treatment with a probiotic, a sample obtained from the digestive tract of a subject may be obtained and the genetic material of the food organisms therein extracted as disclosed herein and subjected to metagenomics analysis. A customized food specific database comprised of whole, partial, or incomplete reference genomes, RNA's, or nucleic acid components or fragments will be utilized by bioinformatics tools to identify, quantify, and taxonomically assign the nucleic acid information from sequencing. The output of which is exemplified in Table 2 below and contains identification of the species of organisms or cells of organisms that were in the gut.

TABLE 2

| | | In Grams | | | | | | | | |
|---|---|---|---|---|---|---|---|---|---|---|
| Scientific | Common | Relative | | | | As a percent of daily recommended | | | | |
| Name | Name | Abundance | Fats | Carbs | Proteins | Iron | Vitamin A | Vitamin B | Vitamin C | Vitamin D |
| Capreolus | Deer | 0.05 | 2.7 | 0 | 26 | 21.0% | | | | |
| capreolus | | | | | | | | | | |
| Raphanus | Raddish | 0.05 | 0 | 0.2 | 0 | 0.0% | 10.0% | | | |
| sativus | | | | | | | | | | |
| Beta | Beet | 0.05 | 0.2 | 13 | 2.2 | 6.0% | 5.0% | 11.0% | | |
| vulgaris | | | | | | | | | | |
| Triticum | Wheat | 0.3 | 4.7 | 137 | 26 | 37.0% | 40.0% | | | |
| aestivum | | | | | | | | | | |
| Corchorus | Jute | 0.05 | 0.2 | 0 | 6 | 15.0% | 90.0% | 25.0% | 47.0% | |
| olitorius | | | | | | | | | | |
| Bos taurus | Cattle | 0.4 | 6 | 0 | 22 | 15.0% | | | | |
| Ipomoea | Sweet | 0.1 | 0.1 | 27 | 2.1 | 4.0% | 377.0% | 15.0% | 5.0% | |
| batatas | Potato | | | | | | | | | |

Then during and/or after treatment with a given probiotic, a second sample may be obtained from the digestive tract of the subject and the genetic material of the microbes in the second sample extracted and subjected to metagenomics analysis, the results of which are compared to the results of the metagenomics analysis of the first sample. Then, based on the comparative results, the food organism results maybe compared to the microbiome organism results to understand the microbes associated with food and an overall food quality assessment. In some embodiments, this may provide information to the species of organism that an individual is ingesting through their food source and any genetic modifications, mutations, or irregularities to the species either by selection or direct modification.

In some embodiments, the second sample of microbiome analysis will enable detection of microbes common to the food organisms and provide information on the health of the food organism. In some embodiments, the human consumed food may be part of the common food source such as chickens, cows, pig, or even plants, and protista where the species will be identified and match to microbes that are specific to them. In particular example embodiments, a chicken species that may have a chicken sarcoma virus may be detected in the second gut microbiome sample analyzed. In some embodiments, the health of a food organism ingested can be determined by the presence or absence of microbes that negatively impact the health of the host organism. In particular example embodiments, a disease, such as Equid herpesvirus 2, which is a respiratory disease in horses, may be detected that may impact the health of a host organism.

In some embodiments, the present invention may be used to screen the gut microbiome of a given subject and then custom tailor a food or diet regime that would enable them to improve the quality of their health for aspects of nutritional balance, improved microbial gut profile, and absorption of nutrients.

### Monitoring Probiotic Treatment

In some embodiments, the present invention may be used to monitor probiotic treatment in subjects. For example, prior to treatment with a probiotic, a sample obtained from the digestive tract of a subject may be obtained and the genetic material of the microbes therein extracted as disclosed herein and subjected to metagenomics analysis. Then during and/or after treatment with a given probiotic, a second sample may be obtained from the digestive tract of the subject and the genetic material of the microbes in the second sample extracted as disclosed herein and subjected to metagenomics analysis, the results of which are compared to the results of the metagenomics analysis of the first sample. Then, based on the comparative results, the probiotic treatment of the subject may be modified to obtain a desired population of microbes in the gut of the subject. For example, a probiotic that comprises a microbe whose amount is desired to be increased in the gut of the subject may be administered to the subject.

In some embodiments, the fecal sample may be mixed or cultured for determination of metabolomic of microbial fecal community. Metabolomic profile can then be used to determine probiotic strains that would benefit the individual. Examples of metabolomic profiles include those affecting energy metabolism, nutrient utilization, insulin resistance, adiposity, dyslipidemia, inflammation, short-chain fatty acids, organic acids, cytokines, neurotransmitters chemicals or phenotype and may include other metabolomic markers.

### Microbiome Screening and Probiotic Selection

The present invention has been successfully used to determine the microbe content of a variety of commercially available probiotics. Additionally, the methods of the present invention are used to determine the microbe content of various probiotics and the microbiome content in the gut of the subject. In one embodiment, based on the microbiome content in the gut of the subject and any desired changes thereto, one may select one or more probiotics that contain the microbes that are desired to be increased and/or maintained in the subject's microbiome health. In one embodiment, based on the microbiome content in the gut of the subject and any desired changes thereto, one may select one or more probiotics that contain the microbes that are desired to be increased and/or maintained in the subject's gut balance in relation to the macronutrient content they are getting from their food source as recorded by survey information from the individual directly or by the present invention of gut organism nucleic acid analysis.

Where the microbiome represents a full picture of their microbiota and the organisms contained in them from bacteria, fungi, viruses, phages, and parasites. For example, using the methods described herein, a subject's gut microbiome is determined to contain 25% A and 75% B, Probiotic 1 is determined to contain 75% A and 25% B and Probiotic 2 is determined to contain 25% A and 75% B. If the subject's gut microbiome is desired to be maintained, one would select Probiotic 2 for administering to the subject. However, if the amounts of A and B in the subject's gut are desired to be 50/50, one may select both Probiotics 1 and 2 to be administered to the subject. Alternatively, one may select Probiotic 1 to be administered to the subject until the amounts of A and B in the subject's gut reaches 50/50. In some embodiments, one may custom tailor a probiotic formulation, e.g., containing equal, varying, or diverse amounts of A and B or other probiotic strains, for administration to the subject. Calculation models utilizing relative abundance of the microbes present in an individual's gut will help determine the type, dose, and cocktail of microbes to include in the probotic. For example, if it is determined that organism A is reduced or absent compared to the general population or previous microbiome analysis, then we would provide probiotic or prebiotics that would increase the concentration of organism A. This prebiotic or probiotic may be the exact organism A or another organism what would support the grown of organism A. The dose given would consider relative abundance of organisms in the individual, performance characteristics of the prebiotic/probiotic such as growth rate, compatibility, receptors or receptor density, genes, or expression patterns, or metabolomic products.

Custom tailored probiotics may not be in equal amounts but are formulated based on relative abundance detected from the individual gut/fecal sample. These formulations are geared to modulate the microbiome to a healthy status. The healthy status of a microbiome is determined by the use of existing aggregate private and public databases such as metaHIT™, Human Microbiome Project™, American Gut Project™, and the like. The healthy status may also be determined individually when a person has no known issues and is in good health, from a blood biomarker checkup perspective, and then has their full microbiome profile completed. After one or several microbiome signatures have been completed then the average of some/all of the microbes found can be understood for that individual and variances from that average can be accessed to determine if they are in dysbiosis. Microbiome profiles can be aggregated into groups that are then assigned a barcode for rapid bioinformatic assignment. Groups can be created by single or multiple phenotypic, diagnostic, or demographic information related to the individual from which the sample was collected from. A unique group can be determined from another group by using statistical models such as linear distance calculations, diversity values, classifiers such as C4.5 decision tree, or principal component analysis an comparing to an aggregate known population such as “normals” defined by the Human Microbiome Project or American Gut Project.

Thus, in some embodiments, the present invention may be used to screen the gut microbiome of a given subject and then custom tailor a probiotic regimen to the given subject based on the subject's gut microbiome.

### Treatment of Dysbiosis

In some embodiments, the present invention may be used to restore a subject's gut flora and/or fauna to homeostasis after an event that has caused a shift in the subject's microbiota from balanced microbiome to one that is causing or may be causing negative side effects, disorders, and/or disease. Health conditions can include but is not limited to various conditions, from acne and allergies, through gastrointestinal ailments, obesity and cancer. One example of such a dysbiosis is in the case of the onset of obesity. Several strains of microbes in the guts of subjects have been shown to be associated with obesity or weight management issues suffered by the subjects. See, e.g., Ley, et al. (2005) PNAS USA 102:11070-11075. For example, in obese animal and human subjects, the ratio of Bacterides to Firmicutes phyla microbes plays an important role in metabolic performance. See, e.g., Turnbaugh, et al. (2012) PLOS ONE 7:e41079. Some gut microbes known to be associated with obesity and weight management issues include Bacteroides uniformis, Bacteroides pectinophilus, Roseburia inulinivorans, Methanobrevibacter smithii, and Bifidobacterium animalis.

Thus, in some embodiments, a ratio of a first given microbe to a second given microbe in the gut of a subject is determined using the methods described herein and then if the ratio is undesired or abnormal, the subject is administered a treatment to modify the ratio to be a desired ratio. In some embodiments, the amount of a first given microbe in a gut of a subject relative to the total amount of all the microbes in the gut of the subject is determined using the methods described herein and then if the relative amount of the first given microbe is undesired or abnormal, the subject is administered a treatment to modify the amount to be a desired amount. Re-testing of their gut microbiome maybe used to determine well they are adhering to the macronutrient and food guidance. Such treatments include administering to the subject: a probiotic containing one or more microbes whose amounts are desired to be increased in the gut of the subject, an antimicrobial agent, e.g., an antibiotic, an antifungal, an antiviral, etc., to kill or slow the growth of a microbe or microbes whose amounts are desired to be decreased in the gut of the subject, a diet and/or a dietary supplement that supports the growth or maintenance of a healthy gut microbiome, e.g., a prebiotic, magnesium, fish oil, L-glutamine, vitamin D, etc., and the like. For example, Million, et al. ((2005) Int. J. Obes. 36:817-825) indicate that the gut microbiota of obese subjects are enriched in Lactobacillus reuteri and depleted in Bifidobacterium animalis and Methanobrevibacter smithii. Therefore, after determining the amounts of Lactobacillus reuteri, Bifidobacterium animalis, and Methanobrevibacter smithii in the gut of a subject using the methods described herein and finding that the amounts are typical or indicative of obesity-associated gut microbiota, the subject may be administered a probiotic containing Bifidobacterium animalis and Methanobrevibacter smithii and relatively little to no amount of Lactobacillus reuteri. In embodiments, the gut microbiota of obese subjects would benefit from foods with flavonoids, polyphenols, and short chain fatty acids.

### Scoring of Your Microbiome

Scoring of the microbiome signature overall uses a similar decision tree, algorithm, artificial intelligence, script, or logic tree as represented in Table 3. This system would enable a score that helps a user understand how healthy their gut microbiome is and if they need to take action on a few or many challenges found. Challenges can include but not limited to, identification of known pathogenic organisms, count and identification of opportunistic pathogens, latent organisms known to cause pathogenic affects when given opportunity, lack of support for good microbial environment but their composition or lack of key strains, overall diversity and count of unique organisms found in top 10 and or organisms with greater than 0.1% prevalence.

Diversity cut offs were determined from an aggregate of sample analysis and a cutoff is determined at x relative abundance. For example, if x=0.1% then 352 unique organisms make up the average healthy profile. Then apply standard deviations around this number and using a Gaussian distribution and percentile under the curve analysis we can score how close to the average diversity number from our database average. The lower your diversity number and further away from the average you are then the less that microbiome would score. The higher the number and the greater your diversity is the more that microbiome would score. This type of scoring categories along with probiotic score will determine a number and visual metered score for the custom to understand how healthy their microbiome is. An example of the graphic visualization is included below. Where low is equal to low microbiome quality and high is equal to high microbiome quality and score. Low->30 out of 100, Med >65 out of 100, High=65 or greater out of 100.

An example of a scoring and probiotic formula algorithm is included in Table 3 below. Table 3 can be represented as decision tree, algorithm, artificial intelligence, script, or logic tree. The function of such decision tree, algorithm, artificial intelligence, script, or logic tree would be output a score of wellness of the individual microbiome as related to probiotics detected and to provide formulation and dosing recommendations for probiotic usage.

An exemplary list of potential categories into which microbes may be grouped is set forth in Table 4 below.

TABLE 3
Example Decision Table for Probiotic Scoring and Formulation.
Includes the Utilization of a Probiotic Strain Database, Metagenomic
Analysis Database, and Literature Curation Database

| Criteria | | Criteria | |
|---|---|---|---|
| Number | Criteria | Answer | Score or Inclusion/Exclusion |
| 1 | Greater than 100 | Yes | If yes then include |
| | reads | | |
| 2 | Greater than 50% of | Yes | |
| | total probiotic reads | | |
| 3 | Greater than 10,000 | Yes | If yes do not include in probiotic formula |
| | reads | | |
| 4 | Greater than 50% of | No | |
| | total reads | | |
| 5 | Greater than 30,000 | Yes | If yes do not include in probiotic formula |
| | reads | | |
| 6 | Greater than 30,000 | Yes | If x > 5 then score +20, x > 3 score 10, |
| | reads for x number | | x > 1 score 5 |
| | of probiotics | | |
| 7 | Total number of | x | If x > 10 then score +20, x > 10 then score 10, |
| | microbes above 100 | | x > 5 score 5 |
| | reads (count) | | |
| 8 | Query for probiotic | Yes | Include in formula at 20 CFU/g or greater |
| | strains and output | | |
| | where 1 = yes and 4 | | |
| | is no and 6 is no and | | |
| | the number of reads | | |
| | is less than 1000 | | |
| 9 | If bacillus | Yes | Do not include |
| 10 | If lactobacillus | Yes | If x > 10000 score +20, if x > 1000 score +10, |
| | acidophilus greater | | if x >100 score +5 |
| | than x reads | | |
| 11 | If bacillus genus | Yes | If x > 1000 score +20, if x > 100 score +10, |
| | greater than x reads | | if x > 10 score +5 |
| 12 | If Saccharomyes | Yes | If x > 1000 score +20, if x > 100 score +10, |
| | boulardi greater than | | if x > 10 score +5 |
| | x reads | | |
| 13 | If infant if nursing | Yes | If x > 0 then score +5, x > 30% then score +10, |
| | and bifidobacterium | | x > 50% then score +20, x > 70% then |
| | infantis >x % | | score +30 |
| 14 | If not infant, not | Yes | If x > 20 then score +5, if x > 10 then score +10, |
| | child and | | if x < 10 then score +20 |
| | bifidobacterium | | |
| | infantis >x % | | |

| | |
|---|---|
| 15 | Query to probiotic function, if function table is equal to health phenotype or |
| | healthDx then include in formula unless 3 or 5 = yes |

TABLE 4
Potential Categories from which to Create Groups

| Categories1 | Categories2 | Categories3 | Categories4 | Categories5 | Categories6 |
|---|---|---|---|---|---|
| ACID_REFLUX | FLOSSING_FREQUENCY | SCIENTIFIC_NAME | VIOSCREEN_D_YOGURT | VIOSCREEN_M_MEAT | VIOSCREEN_VITB12 |
| ACNE_MEDICATION | FLU_VACCINE_DATE | SEAFOOD_FREQUENCY | VIOSCREEN_EER | VIOSCREEN_M_MPF | VIOSCREEN_VITB6 |
| ACNE_MEDICATION_OTC | FROZEN_DESSERT_ | SEASONAL_ALLERGIES | VIOSCREEN_EMAIL | VIOSCREEN_M_NUTSD | VIOSCREEN_VITC |
| | FREQUENCY | | | | |
| ADD_ADHD | FRUIT_FREQUENCY | SEQUENCING_METH | VIOSCREEN_ERYTHR | VIOSCREEN_M_ORGAN | VIOSCREEN_VITD |
| AGE_CAT | FUNGAL_OVERGROWTH | SEX | VIOSCREEN_FAT | VIOSCREEN_M_POULT | VIOSCREEN_VITD2 |
| AGE_CORRECTED | GEO_LOC_NAME | shannon_10k | VIOSCREEN_F_CITMLB | VIOSCREEN_M_SOY | VIOSCREEN_VITD3 |
| AGE_YEARS | GLUTEN | shannon_1k | VIOSCREEN_FIBER | VIOSCREEN_MULTI_ | VIOSCREEN_VITD_IU |
| | | | | CALCIUM_AVG | |
| ALCOHOL_CONSUMPTION | HAS_PHYSICAL_SPECIMEN | SIBO | VIOSCREEN_FIBH2O | VIOSCREEN_MULTI_ | VIOSCREEN_VITE_IU |
| | | | | CALCIUM_DOSE | |
| ALCOHOL_FREQUENCY | HEIGHT_CM | SKIN_CONDITION | VIOSCREEN_FIBINSO | VIOSCREEN_MULTIVITAMIN | VIOSCREEN_VITK |
| ALCOHOL_TYPES | HEIGHT_UNITS | SLEEP_DURATION | VIOSCREEN_FINISHED | VIOSCREEN_MULTIVITAMIN_ | VIOSCREEN_V_ORANGE |
| | | | | FREQ | |
| ALCOHOL_TYPES_ | HIGH_FAT_RED_MEAT_ | SMOKING_FREQUENCY | VIOSCREEN_FISH_ | VIOSCREEN_NATOCO | VIOSCREEN_V_OTHER |
| BEERCIDER | FREQUENCY | | SERVINGS | | |
| ALCOHOL_TYPES_RED_ | HOMECOOKED_MEALS_ | SOFTENER | VIOSCREEN_F_NJ_CITMLB | VIOSCREEN_NCCGLBR | VIOSCREEN_V_POTATO |
| WINE | FREQUENCY | | | | |
| ALCOHOL_TYPES_SOUR_ | HOST_COMMON_NAME | SPECIALIZED_DIET | VIOSCREEN_F_NJ_OTHER | VIOSCREEN_NCCGLGR | VIOSCREEN_V_STARCY |
| BEERS | | | | | |
| ALCOHOL_TYPES_ | HOST_SUBJECT_ID | SPECIALIZED_DIET_ | VIOSCREEN_F_NJ_TOTAL | VIOSCREEN_NIACIN | VIOSCREEN_V_TOMATO |
| SPIRITSHARD_ALCOHOL | | EXCLUDE_DAIRY | | | |
| ALCOHOL_TYPES_ | HOST_TAXID | SPECIALIZED_DIET_ | VIOSCREEN_FOL_DEQV | VIOSCREEN_NIACINEQ | VIOSCREEN_V_TOTAL |
| UNSPECIFIED | | EXCLUDE_NIGHTSHADES | | | |
| ALCOHOL_TYPES_WHITE_ | IBD | SPECIALIZED_DIET_ | VIOSCREEN_FOL_NAT | VIOSCREEN_NITROGEN | VIOSCREEN_WATER |
| WINE | | EXCLUDE_REFINED_SUGARS | | | |
| ALLERGIC_TO | IBD_DIAGNOSIS | SPECIALIZED_DIET_ | VIOSCREEN_FOL_SYN | VIOSCREEN_NON_FRIED_ | VIOSCREEN_WEIGHT |
| | | FODMAP | | FISH_SERVINGS | |
| ALLERGIC_TO_I_HAVE_ | IBD_DIAGNOSIS_REFINED | SPECIALIZED_DIET_HALAAL | VIOSCREEN_FORMONTN | VIOSCREEN_NUTRIENT_ | VIOSCREEN_WGRAIN |
| NO_FOOD_ALLERGIES_ | | | | RECOMMENDATION | |
| THAT_I_KNOW_OF | | | | | |
| ALLERGIC_TO_OTHER | IBS | SPECIALIZED_DIET_I_DO_ | VIOSCREEN_F_OTHER | VIOSCREEN_OMEGA3 | VIOSCREEN_WHOLE_ |
| | | NOT_EAT_A_SPECIALIZED_ | | | GRAIN_SERVINGS |
| | | DIET | | | |
| ALLERGIC_TO_PEANUTS | INSTRUMENT_MODEL | SPECIALIZED_DIET_ | VIOSCREEN_FRIED_FISH_ | VIOSCREEN_OXALIC | VIOSCREEN_XYLITOL |
| | | KOSHER | SERVINGS | | |
| ALLERGIC_TO_SHELLFISH | KIDNEY_DISEASE | SPECIALIZED_DIET_ | VIOSCREEN_FRIED_FOOD_ | VIOSCREEN_OXALICM | VIOSCREEN_ZINC |
| | | MODIFIED_PALEO_DIET | SERVINGS | | |
| ALLERGIC_TO_TREE_ | LACTOSE | SPECIALIZED_DIET_OTHER_ | VIOSCREEN_FRT5_DAY | VIOSCREEN_PANTOTHE | VITAMIN_B_SUPPLEMENT_ |
| NUTS | | RESTRICTIONS_NOT_ | | | FREQUENCY |
| | | DESCRIBED_HERE | | | |
| ALLERGIC_TO_ | LAST_MOVE | SPECIALIZED_DIET_ | VIOSCREEN_FRTSUMM | VIOSCREEN_PECTINS | VITAMIN_D_SUPPLEMENT_ |
| UNSPECIFIED | | PALEODIET_OR_PRIMAL_DIET | | | FREQUENCY |
| ALTITUDE | LAST_TRAVEL | SPECIALIZED_DIET_RAW_ | VIOSCREEN_FRUCTOSE | VIOSCREEN_PFA182 | VIVID_DREAMS |
| | | FOOD_DIET | | | |
| ALZHEIMERS | LATITUDE | SPECIALIZED_DIET_ | VIOSCREEN_FRUIT_ | VIOSCREEN_PFA183 | WATER_LOT |
| | | UNSPECIFIED | SERVINGS | | |
| ANONYMIZED_NAME | LEVEL_OF_EDUCATION | SPECIALIZED_DIET_ | VIOSCREEN_F_TOTAL | VIOSCREEN_PFA184 | WEIGHT_CHANGE |
| | | WESTENPRICE_OR_OTHER_ | | | |
| | | LOWGRAIN_LOW_PROCESSED | | | |
| ANTIBIOTIC_HISTORY | LIBRARY_CONSTRUCTION_ | STATE | VIOSCREEN_GALACTOS | VIOSCREEN_PFA204 | WEIGHT_KG |
| | PROTOCOL | | | | |
| APPENDIX_REMOVED | LINKER | SUBSET_AGE | VIOSCREEN_GAMMTOCO | VIOSCREEN_PFA205 | WEIGHT_UNITS |
| ARTIFICIAL_SWEETENERS | LinkerPrimerSequence | SUBSET_ANTIBIOTIC_ | VIOSCREEN_GENDER | VIOSCREEN_PFA225 | WELL_DESCRIPTION |
| | | HISTORY | | | |
| ASD | LIVER_DISEASE | SUBSET_BMI | VIOSCREEN_GENISTN | VIOSCREEN_PFA226 | WELL_ID |
| ASSIGNED_FROM_GEO | LIVINGWITH | SUBSET_DIABETES | VIOSCREEN_GLAC | VIOSCREEN_PFATOT | WHOLE_EGGS |
| AUTOIMMUNE | LONGITUDE | SUBSET_HEALTHY | VIOSCREEN_GLTC | VIOSCREEN_PHENYLAL | WHOLE_GRAIN_ |
| | | | | | FREQUENCY |
| BarcodeSequence | LOWGRAIN_DIET_TYPE | SUBSET_IBD | VIOSCREEN_GLUCOSE | VIOSCREEN_PHOSPHOR | |
| BIRTH_YEAR | LUNG_DISEASE | SUGAR_SWEETENED_ | VIOSCREEN_GLUTAMIC | VIOSCREEN_PHYTIC | |
| | | DRINK_FREQUENCY | | | |
| BMI | MASTERMIX_LOT | SUGARY_SWEETS_ | VIOSCREEN_GLYCINE | VIOSCREEN_PINITOL | |
| | | FREQUENCY | | | |
| BMI_CAT | MEAT_EGGS_FREQUENCY | SURVEY_ID | VIOSCREEN_GLYCITN | VIOSCREEN_POTASS | |
| BMI_CORRECTED | MENTAL_ILLNESS | TARGET_GENE | VIOSCREEN_G_VWHL | VIOSCREEN_PROCDATE | |
| BODY_HABITAT | MENTAL_ILLNESS_TYPE | TARGET_SUBFRAGMENT | VIOSCREEN_GRAMS | VIOSCREEN_PROLINE | |
| BODY_PRODUCT | MENTAL_ILLNESS_TYPE_ | TAXON_ID | VIOSCREEN_G_TOTAL | VIOSCREEN_PROTANIM | |
| | ANOREXIA_NERVOSA | | | | |
| BODY_SITE | MENTAL_ILLNESS_TYPE_ | TEETHBRUSHING_ | VIOSCREEN_G_WHL | VIOSCREEN_PROTEIN | |
| | BIPOLAR_DISORDER | FREQUENCY | | | |
| BOWEL_MOVEMENT_ | MENTAL_ILLNESS_TYPE_ | THYROID | VIOSCREEN_HEI2010_ | VIOSCREEN_PROTOCOL | |
| FREQUENCY | BULIMIA_NERVOSA | | DAIRY | | |
| BOWEL_MOVEMENT_ | MENTAL_ILLNESS_TYPE_ | TITLE | VIOSCREEN_HEI2010_ | VIOSCREEN_PROTVEG | |
| QUALITY | DEPRESSION | | EMPTY_CALORIES | | |
| BREASTMILK_FORMULA_ | MENTAL_ILLNESS_TYPE_ | TM1000_8_TOOL | VIOSCREEN_HEI2010_ | VIOSCREEN_ | |
| ENSURE | PTSD_POSTTRAUMATIC_ | | FATTY_ACIDS | QUESTIONNAIRE | |
| | STRESS_DISORDER | | | | |
| CANCER | MENTAL_ILLNESS_TYPE_ | TM300_8_TOOL | VIOSCREEN_HEI2010_ | VIOSCREEN_RECNO | |
| | SCHIZOPHRENIA | | FRUIT | | |
| CANCER_TREATMENT | MENTAL_ILLNESS_TYPE_ | TM50_8_TOOL | VIOSCREEN_HEI2010_ | VIOSCREEN_RETINOL | |
| | SUBSTANCE_ABUSE | | GREENS_BEANS | | |
| CARDIOVASCULAR_ | MENTAL_ILLNESS_TYPE_ | TONSILS_REMOVED | VIOSCREEN_HEI2010_ | VIOSCREEN_RGRAIN | |
| DISEASE | UNSPECIFIED | | PROTIEN_FOODS | | |
| CAT | MIGRAINE | TYPES_OF_PLANTS | VIOSCREEN_HEI2010_ | VIOSCREEN_RIBOFLA | |
| | | | REFINED_GRAINS | | |
| CDIFF | MILK_CHEESE_FREQUENCY | VEGETABLE_FREQUENCY | VIOSCREEN_HEI2010_ | VIOSCREEN_SACCHAR | |
| | | | SCORE | | |
| CENSUS_REGION | MILK_SUBSTITUTE_ | VIOSCREEN_A_BEV | VIOSCREEN_HEI2010_ | VIOSCREEN_SALAD_ | |
| | FREQUENCY | | SEAFOODS_PLANT_ | VEGETABLE_SERVINGS | |
| | | | PROTIENS | | |
| CENTER_NAME | MULTIVITAMIN | VIOSCREEN_A_CAL | VIOSCREEN_HEI2010_ | VIOSCREEN_SATOCO | |
| | | | SODIUM | | |
| CENTER_PROJECT_ | NAIL_BITER | VIOSCREEN_ACESUPOT | VIOSCREEN_HEI2010_VEG | VIOSCREEN_SCF | |
| NAME | | | | | |
| chao1_10k | NON_FOOD_ALLERGIES | VIOSCREEN_ACTIVITY_ | VIOSCREEN_HEI2010_ | VIOSCREEN_SCFV | |
| | | LEVEL | WHOLE_FRUIT | | |
| chao1_1k | NON_FOOD_ALLERGIES_ | VIOSCREEN_ADD_SUG | VIOSCREEN_HEI2010_ | VIOSCREEN_SELENIUM | |
| | BEESTINGS | | WHOLE_GRAINS | | |
| CHICKENPOX | NON_FOOD_ALLERGIES_ | VIOSCREEN_ADDSUGAR | VIOSCREEN_HEI_DRK_G_ | VIOSCREEN_SERINE | |
| | DRUG_EG_PENICILLIN | | ORG_VEG_LEG | | |
| CLINICAL_CONDITION | NON_FOOD_ALLERGIES_ | VIOSCREEN_ADSUGTOT | VIOSCREEN_HEI_FRUIT | VIOSCREEN_SFA100 | |
| | PET_DANDER | | | | |
| COLLECTION_DATE | NON_FOOD_ALLERGIES_ | VIOSCREEN_AGE | VIOSCREEN_HEIGHT | VIOSCREEN_SFA120 | |
| | POISON_IVYOAK | | | | |
| COLLECTION_MONTH | NON_FOOD_ALLERGIES_ | VIOSCREEN_ALANINE | VIOSCREEN_HEI_GRAINS | VIOSCREEN_SFA140 | |
| | SUN | | | | |
| COLLECTION_SEASON | NON_FOOD_ALLERGIES_ | VIOSCREEN_ALCOHOL | VIOSCREEN_HEI_MEAT_ | VIOSCREEN_SFA160 | |
| | UNSPECIFIED | | BEANS | | |
| COLLECTION_TIME | observed_otus_10k | VIOSCREEN_ALCOHOL_ | VIOSCREEN_HEI_MILK | VIOSCREEN_SFA170 | |
| | | SERVINGS | | | |
| COLLECTION_TIMESTAMP | observed_otus_1k | VIOSCREEN_ALPHACAR | VIOSCREEN_HEI_NON_ | VIOSCREEN_SFA180 | |
| | | | JUICE_FRT | | |
| COMMON_NAME | OLIVE_OIL | VIOSCREEN_ALPHTOCE | VIOSCREEN_HEI_OILS | VIOSCREEN_SFA200 | |
| CONSUME_ANIMAL_ | ONE_LITER_OF_WATER_ | VIOSCREEN_ALPHTOCO | VIOSCREEN_HEI_SAT_FAT | VIOSCREEN_SFA220 | |
| PRODUCTS_ABX | A_DAY_FREQUENCY | | | | |
| CONTRACEPTIVE | ORIG_NAME | VIOSCREEN_ARGININE | VIOSCREEN_HEI_SCORE | VIOSCREEN_SFA40 | |
| COSMETICS_FREQUENCY | OTHER_SUPPLEMENT_ | VIOSCREEN_ASH | VIOSCREEN_HEI_SODIUM | VIOSCREEN_SFA60 | |
| | FREQUENCY | | | | |
| COUNTRY | PCR_PRIMERS | VIOSCREEN_ASPARTAM | VIOSCREEN_HEI_SOL_FAT_ | VIOSCREEN_SFA80 | |
| | | | ALC_ADD_SUG | | |
| COUNTRY_OF_BIRTH | PD_whole_tree_10k | VIOSCREEN_ASPARTIC | VIOSCREEN_HEI_VEG | VIOSCREEN_SFATOT | |
| COUNTRY_RESIDENCE | PD_whole_tree_1k | VIOSCREEN_AVCARB | VIOSCREEN_HEI_WHL_ | VIOSCREEN_SODIUM | |
| | | | GRAINS | | |
| CSECTION | PETS_OTHER | VIOSCREEN_BCODEID | VIOSCREEN_HISTIDIN | VIOSCREEN_SORBITOL | |
| DEODORANT_USE | PETS_OTHER_FREETEXT | VIOSCREEN_BETACAR | VIOSCREEN_INOSITOL | VIOSCREEN_SRVID | |
| DEPRESSION_BIPOLAR_ | PHYSICAL_SPECIMEN_ | VIOSCREEN_BETACRYP | VIOSCREEN_IRON | VIOSCREEN_STARCH | |
| SCHIZOPHRENIA | LOCATION | | | | |
| DEPTH | PHYSICAL_SPECIMEN_ | VIOSCREEN_BETAINE | VIOSCREEN_ISOLEUC | VIOSCREEN_STARTED | |
| | REMAINING | | | | |
| Description | PKU | VIOSCREEN_BETATOCO | VIOSCREEN_ISOMALT | VIOSCREEN_SUBJECT_ID | |
| DIABETES | PLATFORM | VIOSCREEN_BIOCHANA | VIOSCREEN_JOULES | VIOSCREEN_SUCPOLY | |
| DIABETES_TYPE | PLATING | VIOSCREEN_BMI | VIOSCREEN_JUICE_ | VIOSCREEN_SUCRLOSE | |
| | | | SERVINGS | | |
| DIET_TYPE | POOL_FREQUENCY | VIOSCREEN_CAFFEINE | VIOSCREEN_LACTITOL | VIOSCREEN_SUCROSE | |
| DNA_EXTRACTED | POULTRY_FREQUENCY | VIOSCREEN_CALCIUM | VIOSCREEN_LACTOSE | VIOSCREEN_SWEET_ | |
| | | | | SERVINGS | |
| DOG | PREGNANT | VIOSCREEN_CALCIUM_AVG | VIOSCREEN_LEGUMES | VIOSCREEN_TAGATOSE | |
| DOMINANT_HAND | PREPARED_MEALS_ | VIOSCREEN_CALCIUM_ | VIOSCREEN_LEUCINE | VIOSCREEN_TFA161T | |
| | FREQUENCY | DOSE | | | |
| DRINKING_WATER_ | PRIMER_DATE | VIOSCREEN_CALCIUM_ | VIOSCREEN_LINE_GI | VIOSCREEN_TFA181T | |
| SOURCE | | FREQ | | | |
| DRINKS_PER_SESSION | PRIMER_PLATE | VIOSCREEN_CALCIUM_ | VIOSCREEN_LOW_FAT_ | VIOSCREEN_TFA182T | |
| | | FROM_DAIRY_SERVINGS | DAIRY_SERVING | | |
| ECONOMIC_REGION | PROBIOTIC_FREQUENCY | VIOSCREEN_CALCIUM_ | VIOSCREEN_LUTZEAX | VIOSCREEN_TGRAIN | |
| | | SERVINGS | | | |
| ELEVATION | PROCESSING_ROBOT | VIOSCREEN_CALORIES | VIOSCREEN_LYCOPENE | VIOSCREEN_THIAMIN | |
| ENA-BASE-COUNT | PROJECT_NAME | VIOSCREEN_CARBO | VIOSCREEN_LYSINE | VIOSCREEN_THREONIN | |
| ENA-CHECKLIST | PUBLIC | VIOSCREEN_CHOLEST | VIOSCREEN_MAGNES | VIOSCREEN_TIME | |
| ENA-SPOT-COUNT | QIITA_PREP_ID | VIOSCREEN_CHOLINE | VIOSCREEN_MALTITOL | VIOSCREEN_TOTALTFA | |
| ENV_BIOME | QIITA_STUDY_ID | VIOSCREEN_CLAC9T11 | VIOSCREEN_MALTOSE | VIOSCREEN_TOTCLA | |
| ENV_FEATURE | RACE | VIOSCREEN_CLAT10C12 | VIOSCREEN_MANGAN | VIOSCREEN_TOTFOLAT | |
| ENV_MATERIAL | READY_TO_EAT_MEALS_ | VIOSCREEN_COPPER | VIOSCREEN_MANNITOL | VIOSCREEN_TOTSUGAR | |
| | FREQUENCY | | | | |
| ENV_MATTER | RED_MEAT_FREQUENCY | VIOSCREEN_COUMEST | VIOSCREEN_M_EGG | VIOSCREEN_TRYPTOPH | |
| ENV_PACKAGE | REQUIRED_SAMPLE_INFO_ | VIOSCREEN_CYSTINE | VIOSCREEN_METHHIS3 | VIOSCREEN_TYROSINE | |
| | STATUS | | | | |
| EPILEPSY_OR_SEIZURE_ | ROOMMATES | VIOSCREEN_DAIDZEIN | VIOSCREEN_METHION | VIOSCREEN_USER_ID | |
| DISORDER | | | | | |
| EXERCISE_FREQUENCY | ROOMMATES_IN_STUDY | VIOSCREEN_DATABASE | VIOSCREEN_MFA141 | VIOSCREEN_VALINE | |
| EXERCISE_LOCATION | RUNCENTER | VIOSCREEN_D_CHEESE | VIOSCREEN_MFA161 | VIOSCREEN_V_DRKGR | |
| EXPERIMENT_CENTER | RUNDATE | VIOSCREEN_DELTTOCO | VIOSCREEN_MFA181 | VIOSCREEN_VEG5_DAY | |
| EXPERIMENT_DESIGN_ | RUN_PREFIX | VIOSCREEN_DISCFAT_OIL | VIOSCREEN_MFA201 | VIOSCREEN_VEGETABLE_ | |
| DESCRIPTION | | | | SERVINGS | |
| EXPERIMENT_TITLE | SALTED_SNACKS_ | VIOSCREEN_D_ISCFAT_SOL | VIOSCREEN_MFA221 | VIOSCREEN_VEGSUMM | |
| | FREQUENCY | | | | |
| EXTRACTIONKIT_LOT | #Sample ID | VIOSCREEN_D_MILK | VIOSCREEN_MFATOT | VIOSCREEN_VISIT | |
| EXTRACTION_ROBOT | SAMPLE_PLATE | VIOSCREEN_DOB | VIOSCREEN_M_FISH_HI | VIOSCREEN_VITA_IU | |
| FED_AS_INFANT | SAMPLE_TYPE | VIOSCREEN_D_TOTAL | VIOSCREEN_M_FISH_LO | VIOSCREEN_VITA_RAE | |
| FERMENTED_PLANT_ | SAMP_SIZE | VIOSCREEN_D_TOT_SOYM | VIOSCREEN_M_FRANK | VIOSCREEN_VITA_RE | |
| FREQUENCY | | | | | |

Additional Tables are provided below describing microbiome populations and linkage of microbes of the gut to disease.

TABLE 5
List of Strains of Gut Bacteria That Can be Used to Restore Conditions and Profiles of the Microbial Ecosystems

| | | | | | Types of | |
|---|---|---|---|---|---|---|
| | | Combination | | Normal healthy | profiles that | |
| Organism | Organism | species it | Prebiotic or | profile | organism will | Indications |
| Genus | species | works with | Postbiotics | identified in | benefit | for use |
| Akkermansia | muciniphila | Methanobevibacter | Resistant starches, | SG455 | Obesity, | Increased |
| | KLE1798, | smithii, | inulin, chicory | SG449 | Weight Loss, | metabolism |
| | KLE1797, | Faecalibacterium | root, | | Chronic | and weight |
| | KLE160 | prausnitzii | oligosacharrides, | | Fatigue, Low | loss or |
| | | Roseburia | soluble fiber | | Energy | management. |
| | | hominis, | | | | |
| | | Prevotella copri | | | | |
| Faecalibacterium | prausnitzii | Bacteroides | Frucoto- | SG216 | Low | IBD with low |
| | strain A2165, | ovatus, | oligosaccharide, | SG242 | Bacteroides | Faecalibacterium, |
| | M21/2, | Bacteroides | inulin, other | | genus, | Low |
| | KLE1255, | uniformis, | oligosaccharides | | infectious | Bacteroides, |
| | | Bacteroides | | | microbes like | High |
| | | caccae | | | C. diff, | Bifidobacterium |
| | | | | | sutterella | |
| | | | | | wadsworthenis, | |
| | | | | | or | |
| | | | | | blastocysthominis, | |
| | | | | | plasmodium | |
| Methanobrevibacter | Smithii | Akkermansia | Resveratrol, green | SG210 | Endurance | Remove |
| | TS94C | muciniphila, | tea extract, other | | athletes, active | bloating and |
| | | Faecalbacterium | herbs, | | individuals, | side effects of |
| | | prausnitzii, | oligosaccharides, | | those that have | Dorea, |
| | | Roserburia hominis, | and biochemicals | | Oderibacteri, | Reumminococus, |
| | | Bacteroides | | | or | and other |
| | | uniformis | | | Akkermansia | gut microbes |
| | | | | | at | that produce |
| | | | | | concnetrations | Alcohols, H2O |
| | | | | | of Resveratrol | |
| | | | | | 0.1% or | |
| | | | | | greater relative | |
| | | | | | abundance | |
| Bifidobacterium | Pseudocatenulatum | Other probiotic | Natural herbs and | SG178 | People with | |
| | IPLA36007, | strains just at | extracts that | | depression, | |
| | DSM 20438, | B. animalis lactis, | increase dopamine | | low probiotic | |
| | Catenulatum | B animalis infantis, | or serotonin as well | | diversity with | |
| | DSM 16992 | Strephococcus thermophilus | as XOS that | | probiotics | |
| | | and L. planantarum | support growth of | | below 0.1 | |
| | | | Bifidobacterium | | | |
| Bacteroides | xylanisolvens | Bacteroides ovatus, | Xylans, xylitol, | SG338 | vegetarian and | |
| | CL03T12C04 | caccae, uniformis, | plant celluloses or | SG352 | plant based | |
| | | xylanisolvens, | other synthetic | | diet to enable | |
| | | Butyrivibrio | celluloses | | advanced | |
| | | crossotus | | | nutritional | |
| | | | | | absorption | |
| | | | | | from plant | |
| | | | | | based food | |
| Bacteroides | cellulitis | Bacteroides ovatus, | Plant celluloses or | SG338 | vegetarian and | |
| | | caccae, uniformis, | other synthetic | SG352 | plant based | |
| | | xylanisolvens, | celluloses, plant | | diet to enable | |
| | | Butyrivibrio | based fibers, | | advanced | |
| | | crossotus | | | nutritional | |
| | | | | | absorption | |
| | | | | | from plant | |
| | | | | | based food | |
| Anaerostipes | Hadrus DSM | Facalibacterium | Walnuts, | SG279 | potential for | |
| | 3319 | prausnitzii, | Resveratrol | | diabetes or | |
| | | Saccharomyes | | | weight loss. If | |
| | | boulardii | | | you have | |
| | | | | | Oscillibacter | |
| | | | | | organisms that | |
| | | | | | are high but do | |
| | | | | | not have any | |
| | | | | | IBD | |
| | | | | | phenotypes. | |
| | | | | | Cannot be low | |
| | | | | | on | |
| | | | | | Faecalibacterium, | |
| | | | | | helpful in | |
| | | | | | MS or leaky | |
| | | | | | gut scenarios, | |
| | | | | | removal of | |
| | | | | | reducing sugar | |
| Butyrivibrio | Crossotus | Facalibacterium | Walnuts, | SG279 | Increase | |
| | DSM 2876 | prausnitzii, | Resveratrol | | butyrate, heal | |
| | | Bacteroides ovatus, | | | leaky gut, | |
| | | caccae, uniformis, | | | reduce | |
| | | cellulitis | | | bacteremia of | |
| | | | | | blood, digest | |
| | | | | | plant based | |
| | | | | | foods | |
| Gemmiger | formicilis | Facalibacterium | Walnuts | SG104, | Profiles with | |
| | | prausnitzii | Resveratrol | SG179 | Bacteroides | |
| | | | Saccharomyces | | vulgatus above | |
| | | | boulardii | | 10% or fragilis | |
| | | | | | above 5%, | |
| Roseburia | faecis | Roseburia | Resistant starches, | SG242 | IBD, IBS, C- | |
| | | hominis + | dietary fiber, | SG104 | diff or other | |
| | | Roseburia | Bifidobacterium | | gut | |
| | | intestinalis + | pseudocatenulatum | | inflammation | |
| | | Roseburia | Catenulatum, | | issues, | |
| | | inulinivorans | Dopamine, | | butyrate- | |
| | | | Vitamin D, fungi, | | producing | |
| | | | yeasts, algae, | | bacteria - | |
| | | | lichens, and plants, | | anti- | |
| | | | such as oats and | | inflammatory, | |
| | | | barley. | | explore for | |
| | | | | | autoimmune | |
| | | | | | issues | |
| Roseburia | Inulinivorans | Roseburia | Resistant starches, | SG179 | IBD, IBS, C- | |
| | DSM 16841, | hominis + | dietary fiber, | | diff or other | |
| | | Roseburia | Bifidobacterium | | gut | |
| | | intestinalis + | pseudocatenulatum | | inflammation | |
| | | Roseburia | Catenulatum, | | issues, | |
| | | faecis | Dopamine, | | butyrate- | |
| | | | Vitamin D, fungi, | | producing | |
| | | | yeasts, algae, | | bacteria - | |
| | | | lichens, and plants, | | anti- | |
| | | | such as oats and | | inflammatory, | |
| | | | barley. | | explore for | |
| | | | | | autoimmune | |
| | | | | | issues | |
| Roseburia | Hominis | Roseburia | Resistant starches, | SG179 | IBD, IBS, C- | |
| | A2-183, | intestinalis + | dietary fiber, | | diff or other | |
| | | Roseburia | Bifidobacterium | | gut | |
| | | faecis + | pseudocatenulatum | | inflammation | |
| | | Roseburia | Catenulatum, | | issues, | |
| | | inulinivorans | Dopamine, | | butyrate- | |
| | | | Vitamin D, fungi, | | producing | |
| | | | yeasts, algae, | | bacteria - | |
| | | | lichens, and plants, | | anti- | |
| | | | such as oats and | | inflammatory, | |
| | | | barley. | | explore for | |
| | | | | | autoimmune | |
| | | | | | issues es | |
| Roseburia | intestinalis | Roseburia | Resistant starches, | SG242 | IBD, IBS, C- | |
| | | hominis + | dietary fiber, | | diff or other | |
| | | Roseburia | Bifidobacterium | | gut | |
| | | inulinivorans + | pseudocatenulatum | | inflammation | |
| | | Roseburia | Catenulatum, | | issues, | |
| | | faecis | Dopamine, | | butyrate- | |
| | | | Vitamin D, fungi, | | producing | |
| | | | yeasts, algae, | | bacteria - | |
| | | | lichens, and plants, | | anti- | |
| | | | such as oats and | | inflammatory, | |
| | | | barley. | | explore for | |
| | | | | | autoimmune | |
| | | | | | issues | |
| Bacteroides | uniformis | Bacteroides ovatus, | Fructo- | SG104, | IBD, IBS, | |
| | dnLKV2, etc | caccae, | oligosaccharides, | SG179, | gluten | |
| | | Facalibacterium | resistant starches, | SG279, | sensitivity or | |
| | | prausnitzii, | red wine, L- | SG441, | intolerance, | |
| | | Roseburia | citrulline | SG338, | food allergies | |
| | | species, | | SG352 | | |
| | | Prevotella copri, | | | | |
| | | Eubacterium | | | | |
| | | species | | | | |
| Bacteroides | ovatus | Bacteroides uniformis, | Fructo- | SG104, | IBD, IBS, | |
| | | caccae, | oligosaccharides, | SG179, | gluten | |
| | | Facalibacterium | resistant starches, | SG279, | sensitivity or | |
| | | prausnitzii, | red wine, L- | SG441, | intolerance, | |
| | | Roseburia | citrulline | SG338, | food allergies | |
| | | species, | | SG352 | | |
| | | Prevotella copri, | | | | |
| | | Eubacterium | | | | |
| | | species | | | | |
| Prevotella | copri | Bacteroides ovatus, | Saccharomyces | SG441 | In cases of | |
| | | caccae, | organisms, | | high | |
| | | Facalibacterium | legumes and | | Bacteroides | |
| | | prausnitzii, | lentils, | | fragilis or | |
| | | Roseburia | Mediterranean diet, | | vulgatus when | |
| | | species, | flaxseed, | | you shift to | |
| | | Eubacterium | | | low saturated | |
| | | species, | | | fat diet or | |
| | | Saccharomyces | | | vegetarian | |
| | | organisms, | | | diet, | |
| | | | | | Intolerances to | |
| | | | | | legumes or | |
| | | | | | plant based | |
| | | | | | food. | |
| Bifidobacterium | kashiwanohense | Bifidobacterium | Natural herbs and | SG441 | People with | |
| | PV20-2, | longum sp | extracts that | | depression, | |
| | DSM 21854, | infantis, | increase dopamine | | low probiotic | |
| | JCM 15439 | Bifidobacterium | or serotonin as well | | diversity, | |
| | etc | | as XOS that | | mood | |
| | | | support growth of | | disorders, with | |
| | | | Bifidobacterium | | probiotics | |
| | | | | | below 0.1 | |
| Papillibacter | cinnamivorans | Bacteroides | Cinnamon oils, | SG441 | Vegetarians, | |
| | DSM | cellulitis, | shea butter, | | people with | |
| | 12816, etc | xylanisolvens | balsams | | trouble eating | |
| | | | | | spicy foods, | |
| | | | | | trouble with | |
| | | | | | eating plants | |
| Lactobacillus | ruminis | Lactobacillus | Oligo-saccharides | SG441 | Reduction of | |
| | ATCC 25644, | acidophilus, | (mono-, di-, tri-, | | pathogens | |
| | L5, S21, S23, | plantarum, | and tetra-) and | | such as E. coli, | |
| | S36, S38, etc | reuteri, | other | | C. duff, | |
| | | delbrueckii, and | oligosaccharides, | | Shigella, | |
| | | other organism | lactose, lactulose, | | Salmonella, | |
| | | that have been | GOS inulin or | | and other toxic | |
| | | shown to reduce | other inulins | | Bacteroides | |
| | | gut microbes | | | fragilis or | |
| | | that have | | | valgatus | |
| | | negative | | | organisms that | |
| | | impacts | | | are motile | |
| Oxalobacter | formigenes | Lactobacillus | Calcium, | SG441 | Reduce the | |
| | HOxBLS, etc | acidophilus, | Magnesium, | | kidney stone | |
| | | Oxalobacter | Vitamins (like | | build up, help | |
| | | vibrioformis | vitamin C) | | with mineral | |
| | | | | | metabolism, | |
| | | | | | reduce risk of | |
| | | | | | hyperoxaluria | |
| | | | | | and metabolic | |
| | | | | | disorders | |
| Bacteroides | caccae | Bacteroides | Fructo- | SG104, | IBD, IBS, | |
| | CL03T12C61, | uniformis, | oligosaccharides, | SG179, | gluten | |
| | ATCC | ovatus, | resistant starches, | SG279, | sensitivity or | |
| | 43185 etc | Facalibacterium | red wine, L- | SG441, | intolerance, | |
| | | prausnitzii, | citrulline | SG338, | food allergies | |
| | | Roseburia | | SG352 | | |
| | | species, | | | | |
| | | Prevotella | | | | |
| | | copri, | | | | |
| | | Eubacterium | | | | |
| | | species | | | | |
| Eubacterium | rectale ATCC | Eubacterium siraeum, | Inulin, dietary | SG433 | Constipation, | |
| | 33656, etc | ramulus, | fibers, resistant | | chronic | |
| | | eligens, hallii, | starches, raw | | fatigue, | |
| | | Akkermansia | banana extract, | | lethargy, | |
| | | muciniphilia | | | irregular | |
| | | | | | bowel | |
| | | | | | movements, | |
| | | | | | poor Bristol | |
| | | | | | score for | |
| | | | | | movements, | |
| | | | | | leaky gut, IBS | |
| Eubacterium | siraeum DSM | Eubacterium | Inulin, dietary | SG441 | Constipation, | |
| | 15702 | rectale, | fibers, resistant | | chronic | |
| | | ramulus, | starches | | fatigue, | |
| | | eligens, hallii, | | | lethargy, | |
| | | Akkermansia | | | irregular | |
| | | muciniphilia | | | bowel | |
| | | | | | movements, | |
| | | | | | poor Bristol | |
| | | | | | score for | |
| | | | | | movements, | |
| | | | | | leaky gut, IBS | |
| Eubacterium | eligens | Eubacterium | Inulin, dietary | SG433 | Constipation, | |
| | ATCC 27750 | rectale, | fibers, resistant | | chronic | |
| | | ramulus, | starches | | fatigue, | |
| | | siraeum, hallii, | | | lethargy, | |
| | | Akkermansia | | | irregular | |
| | | muciniphilia | | | bowel | |
| | | | | | movements, | |
| | | | | | poor Bristol | |
| | | | | | score for | |
| | | | | | movements, | |
| | | | | | leaky gut, IBS | |
| Eubacterium | hallii DSM | Eubacterium | Inulin, dietary | SG433 | Constipation, | |
| | 3353 | rectale, | fibers, resistant | | chronic | |
| | | ramulus, | starches | | fatigue, | |
| | | siraeum, | | | lethargy, | |
| | | eligens, | | | irregular | |
| | | Akkermansia | | | bowel | |
| | | muciniphilia | | | movements, | |
| | | | | | poor Bristol | |
| | | | | | score for | |
| | | | | | movements, | |
| | | | | | leaky gut, IBS | |
| Butyricicoccus | pullicaecorum | | | | | |

TABLE 6
List of Strains of Gut Bacteria that are Causative Microbes Impacting the Microbiome and Gut Health of an Individual

| | | | Prebiotic or | | | |
|---|---|---|---|---|---|---|
| | | | Postbiotics to | Disorder | | |
| | | Combination | reduce | profile | | |
| Organism | Organism | species it works | causative | identified | Risk of | |
| Genus | species | with | microbe | in | disease | Indications |
| Oscillibacter | ruminantium | Other | Avoid Resistant | SG355 | Cardiovascular | Type 2 |
| | | Oscillibacter, | starches | | disease risk | diabetes risk |
| | | | | | when above | and weight |
| | | | | | 0.5% | management |
| | | | | | | issues |
| Bacteroides | vulgatus | Any | Avoid XOS | SG372 | Both vulgatus | Irritable |
| | AND/OR | inflammatory | | | and fragilis are | Bowel |
| | fragilis | microbe such as | | | above 5% or one | Syndrome |
| | | those of the | | | is above 10% | |
| | | genus | | | | |
| | | Peptoclostridium | | | | |
| | | Escherichia | | | | |
| | | Klebsiella | | | | |
| Bacteroides | Overall | Faecalibacterium | Bacillus | | When | IBD |
| | with vulgatus | reduced | Coagulans | | Faecalibacterium | Ulcerative |
| | and fragilis high | | Bifidobacterium | | is below 15%, | colitis |
| | | | breve | | | |
| | | | L. casei | | | |
| | | | S. boulardii | | | |
| | | | Lactobacillus | | | |
| | | | plantarum | | | |
| | | | prebiotic fibers | | | |
| Clostridium | Boltea | Akkermansia | Lactobacillus | SG314 | When mucin | Mood |
| Plasmodium | Various | muciniphilia, | rhamnosus GG | SG213 | degrading | Disorder |
| Cryptosporidium | Various | Cloacibacillus | Lactobacillus casei | | microbes are | |
| Blastocystis | Various | porcorum | B. breve | | present along with | |
| Penicillium | Various | | Other microbes | | pathogenic | |
| Botulism | excrementihomini | | that support the | | microbes | |
| Parasutterella | | | growth of | | | |
| | | | B. psedocatenlaum | | | |
| Alistipes | Various | Pathogens like | Reduce | SG280 | When any one | Small |
| Bacteriodes | | Klebsiella, | saturated fat | SG346 | microbe is | Intestinal |
| Escherichia | | Salmonella, | intake, reduce | | present at greater | Bacteria |
| Prevotella | | Shigella | refined sugar | | than 25% | Overgrowth |
| Ruminococcus | | | intake, add | | relative | |
| | | | soluable fibers | | abundance | |
| | | | to diet | | | |
| Klebsiella | psuenomiae | | Reduce | SG179 | EARLY or | Cancer |
| Escherichia | variicola | | constipation | SG95 | PRE Decreased | injury or |
| Salmonella | michiganensis | | B. Infantis, | | Roseburia | post |
| Shigella | oxytoca | | Bifidobacterium | | organisms, | surgery, |
| | quasipneumoniae | | animalis | | Bacteroides | |
| | coli | | B. longum | | vulgatus and | |
| | enterica | | | | fragilis above | |
| | | | | | 5% along with | |
| | | | | | Escherichia | |
| | | | | | OR LATE | |
| | | | | | STAGE | |
| | | | | | Reduce | |
| | | | | | Bacteroides | |
| | | | | | genus | |
| | | | | | Increased | |
| | | | | | Rosburia | |
| | | | | | concentrations | |
| Escherichia | coli | Bacteroides | | SG841 | When E. coli is | IBS-D |
| | | vulgatus, | | | above 2.5% | |
| | | fragilis; | | | combined with | |
| | | Klebsiella | | | elevated level of | |
| | | pneumonia, | | | Bacteroides, | |
| | | variicola | | | Klebsiella, or | |
| | | | | | Pseudomonas | |
| | | | | | organisms | |
| Clostridium | difficile | Bacteroides | | SG845 | Symptoms of | IBS-C |
| Enterobacteria | cloacae | vulgatus, | | | fatigue, | |
| | | fragilis; | | | constipation, | |
| | | Candida; | | | arthritis, GERD, | |
| | | Klebsiella | | | stomach pain. | |
| | | | | | C. diff is above | |
| | | | | | 1.8% | |

### Tables 7-10

### Example of a Complete GutBuster™ Report Detailing the Abundance of Bacterioides, Archaea, Eukaryotes, Viruses, and Bacteria in an Exemplary Stool Sample

TABLE 7
Report of the Abundance of Bacterioides

| | | | Assigned | Assigned | | Corrected |
|---|---|---|---|---|---|---|
| Taxonomy | Taxonomy | | reads | reads | Relative | relative |
| levels | ID | Organisms | (tree) | (taxon) | abundances | abundances |
| S | 820 | Bacteroides uniformis | 1398898 | 1398898 | 10.24 | 13.11 |
| S | 214856 | Alistipes finegoldii | 683209 | 683209 | 5 | 6.4 |
| S | 46506 | Bacteroides stercoris | 656815 | 656815 | 4.81 | 6.15 |
| S | 821 | Bacteroides vulgatus | 570952 | 570952 | 4.18 | 5.35 |
| S | 287 | Pseudomonas aeruginosa | 464292 | 464292 | 3.4 | 4.35 |
| S | 562 | Escherichia coli | 460793 | 460793 | 3.37 | 4.32 |
| S | 28116 | Bacteroides ovatus | 376631 | 376631 | 2.76 | 3.53 |
| S | 39492 | [Eubacterium] siraeum | 348382 | 348382 | 2.55 | 3.26 |
| S | 817 | Bacteroides fragilis | 308172 | 308172 | 2.26 | 2.89 |
| S | 28117 | Alistipes putredinis | 291675 | 291675 | 2.14 | 2.73 |
| S | 328814 | Alistipes shahii | 246655 | 246655 | 1.81 | 2.31 |
| S | 46503 | Parabacteroides merdae | 244990 | 244990 | 1.79 | 2.3 |
| S | 853 | Faecalibacterium prausnitzii | 238187 | 238187 | 1.74 | 2.23 |
| S | 1150298 | Fusicatenibacter saccharivorans | 210553 | 210553 | 1.54 | 1.97 |
| S | 328813 | Alistipes onderdonkii | 196619 | 196619 | 1.44 | 1.84 |
| S | 33039 | [Ruminococcus] torques | 194902 | 194902 | 1.43 | 1.83 |
| S | 239935 | Akkermansia muciniphila | 177149 | 177149 | 1.3 | 1.66 |
| S | 39491 | [Eubacterium] rectale | 172572 | 172572 | 1.26 | 1.62 |
| S | 1581179 | Clostridium sp. HM5C19A11 | 138737 | 138737 | 1.02 | 1.3 |
| S | 908612 | Alistipes sp. HGBS | 119109 | 119109 | 0.87 | 1.12 |
| S | 823 | Parabacteroides distasonis | 113923 | 113923 | 0.83 | 1.07 |
| S | 47678 | Bacteroides caccae | 107493 | 107493 | 0.79 | 1.01 |
| S | 1463165 | Klebsiella quasipneumoniae | 103357 | 103357 | 0.76 | 0.97 |
| S | 410072 | Coprococcus comes | 85711 | 85711 | 0.63 | 0.8 |
| S | 457394 | Bacteroides sp. 4_3 47FAA | 82836 | 82836 | 0.61 | 0.78 |
| S | 1496 | Clostridioides difficile | 68751 | 68751 | 0.5 | 0.64 |
| S | 28118 | Odoribacter splanchnicus | 63959 | 63959 | 0.47 | 0.6 |
| S | 328812 | Parabacteroides goldsteinii | 54004 | 54004 | 0.4 | 0.51 |
| S | 1499682 | Alistipes sp. AL-1 | 53142 | 53142 | 0.39 | 0.5 |
| S | 418240 | Blautia wexlerae | 52500 | 52500 | 0.38 | 0.49 |
| S | 649756 | Anaerostipes hadrus | 48915 | 48915 | 0.36 | 0.46 |
| S | 88431 | Dorea longicatena | 47143 | 47143 | 0.35 | 0.44 |
| S | 1118061 | Alistipes obesi | 45000 | 45000 | 0.33 | 0.42 |
| S | 469591 | Parabacteroides sp. 20_3 | 44426 | 44426 | 0.33 | 0.42 |
| S | 35833 | Bilophila wadsworthia | 44069 | 44069 | 0.32 | 0.41 |
| S | 585543 | Bacteroides sp. D20 | 43700 | 43700 | 0.32 | 0.41 |
| S | 1715163 | Pseudomonas sp. HM5C058A10 | 42905 | 42905 | 0.31 | 0.4 |
| S | 818 | Bacteroides thetaiotaomicron | 42205 | 42205 | 0.31 | 0.4 |
| S | 1504823 | bacterium LF-3 | 41024 | 41024 | 0.3 | 0.38 |
| S | 658662 | Parabacteroides sp. D26 | 40182 | 40182 | 0.29 | 0.38 |
| S | 166486 | Roseburia intestinalis | 37299 | 37299 | 0.27 | 0.35 |
| S | 556258 | Bacteroides sp. D1 | 37178 | 37178 | 0.27 | 0.35 |
| S | 573 | Klebsiella pneumoniae | 35661 | 35661 | 0.26 | 0.33 |
| S | 29348 | [Clostridium] spiroforme | 33592 | 33592 | 0.25 | 0.31 |
| S | 1470347 | Alistipes ihumii | 32731 | 32731 | 0.24 | 0.31 |
| S | 469593 | Bacteroides sp. 3_1 40A | 32544 | 32544 | 0.24 | 0.3 |
| S | 39488 | [Eubacterium] hallii | 32172 | 32172 | 0.24 | 0.3 |
| S | 40520 | Blautia obeum | 31062 | 31062 | 0.23 | 0.29 |
| S | 39486 | Dorea formicigenerans | 30806 | 30806 | 0.23 | 0.29 |
| S | 169435 | Anaerotruncus colihominis | 30706 | 30706 | 0.22 | 0.29 |
| S | 665949 | Tannerella sp. 6_1_58FAA_CT1 | 29336 | 29336 | 0.21 | 0.27 |
| S | 1297617 | Intestinimonas butyriciproducens | 28667 | 28667 | 0.21 | 0.27 |
| S | 100886 | Catenibacterium mitsuokai | 28587 | 28587 | 0.21 | 0.27 |
| S | 291645 | Bacteroides nordii | 28552 | 28552 | 0.21 | 0.27 |
| S | 745368 | Gemmiger formicilis | 27814 | 27814 | 0.2 | 0.26 |
| S | 357276 | Bacteroides dorei | 26184 | 26184 | 0.19 | 0.25 |
| S | 39485 | [Eubacterium] eligens | 25952 | 25952 | 0.19 | 0.24 |
| S | 360807 | Roseburia inulinivorans | 24453 | 24453 | 0.18 | 0.23 |
| S | 1099853 | Coprobacter fastidiosus | 23908 | 23908 | 0.18 | 0.22 |
| S | 1531 | [Clostridium] clostridioforme | 23228 | 23228 | 0.17 | 0.22 |
| S | 292800 | Flavonifractor plautii | 23050 | 23050 | 0.17 | 0.22 |
| S | 74426 | Collinsella aerofaciens | 21999 | 21999 | 0.16 | 0.21 |
| S | 301302 | Roseburia faecis | 21335 | 21335 | 0.16 | 0.2 |
| S | 457393 | Bacteroides sp. 4_1_36 | 19511 | 19511 | 0.14 | 0.18 |
| S | 592978 | Ruminococcus faecis | 19271 | 19271 | 0.14 | 0.18 |
| S | 469592 | Bacteroides sp. 3_1_19 | 19051 | 19051 | 0.14 | 0.18 |
| S | 1535 | [Clostridium] leptum | 17531 | 17531 | 0.13 | 0.16 |
| S | 665956 | Subdoligranulum sp. 4_3_54A2FAA | 17450 | 17450 | 0.13 | 0.16 |
| S | 310298 | Bacteroides coprocola | 16870 | 16870 | 0.12 | 0.16 |
| S | 1737424 | Blautia massiliensis | 16078 | 16078 | 0.12 | 0.15 |
| S | 1432052 | Eisenbergiella tayi | 15741 | 15741 | 0.12 | 0.15 |
| S | 469590 | Bacteroides sp. 2_2_4 | 15107 | 15107 | 0.11 | 0.14 |
| S | 1288121 | Alistipes senegalensis | 14730 | 14730 | 0.11 | 0.14 |
| S | 487175 | Parasutterella excrementihominis | 14498 | 14498 | 0.11 | 0.14 |
| S | 571438 | Desulfovibrio legallii | 13019 | 13019 | 0.1 | 0.12 |
| S | 469610 | Burkholderiales bacterium 1_1_47 | 12688 | 12688 | 0.09 | 0.12 |
| S | 1519439 | Oscillibacter sp. ER4 | 12032 | 12032 | 0.09 | 0.11 |
| S | 1661050 | Pseudomonas sp. NBRC 111135 | 11809 | 11809 | 0.09 | 0.11 |
| S | 33038 | [Ruminococcus] gnavus | 11385 | 11385 | 0.08 | 0.11 |
| S | 1550024 | Ruthenibacterium lactatiformans | 11146 | 11146 | 0.08 | 0.1 |
| S | 371601 | Bacteroides xylanisolvens | 10839 | 10839 | 0.08 | 0.1 |
| S | 1520805 | Blautia sp. SF-50 | 10647 | 10647 | 0.08 | 0.1 |
| S | 1882754 | Clostridium sp. DSM 4029 | 10556 | 10556 | 0.08 | 0.1 |
| S | 2025876 | Parabacteroides sp. CTO6 | 10490 | 10490 | 0.08 | 0.1 |
| S | 1226324 | Blautia sp. KLE 1732 | 9880 | 9880 | 0.07 | 0.09 |
| S | 1406512 | Candidatus Methanomassiliicoccus intestinalis | 9667 | 9667 | 0.07 | 0.09 |
| S | 1078089 | Bacteroides sp. HPS0048 | 8594 | 8594 | 0.06 | 0.08 |
| S | 658657 | Erysipelotrichaceae bacterium 21_3 | 8061 | 8061 | 0.06 | 0.08 |
| S | 1095771 | Ruminococcus sp. JC304 | 8047 | 8047 | 0.06 | 0.08 |
| S | 623 | Shigella flexneri | 7995 | 7995 | 0.06 | 0.07 |
| S | 626929 | Bacteroides clarus | 7860 | 7860 | 0.06 | 0.07 |
| S | 457412 | Ruminococcus sp. 5_1_39BFAA | 7782 | 7782 | 0.06 | 0.07 |
| S | 208479 | [Clostridium] bolteae | 7763 | 7763 | 0.06 | 0.07 |
| S | 387661 | Parabacteroides johnsonii | 7730 | 7730 | 0.06 | 0.07 |
| S | 2024228 | Prevotella sp. P4-51 | 7505 | 7505 | 0.05 | 0.07 |
| S | 1805476 | Blautia sp. Marseille-P2398 | 7495 | 7495 | 0.05 | 0.07 |
| S | 160404 | [Clostridium] lactatifermentans | 7423 | 7423 | 0.05 | 0.07 |
| S | 39490 | Eubacterium ramulus | 7338 | 7338 | 0.05 | 0.07 |
| S | 450746 | Coprobacillus sp. 8_1_38FAA | 7261 | 7261 | 0.05 | 0.07 |
| S | 1232457 | Clostridiales bacterium VE202-27 | 7092 | 7092 | 0.05 | 0.07 |
| S | 419005 | Prevotella amnii | 6970 | 6970 | 0.05 | 0.07 |
| S | 457402 | Eubacterium sp. 3_1_31 | 6724 | 6724 | 0.05 | 0.06 |
| S | 501571 | Butyricicoccus pullicaecorum | 6714 | 6714 | 0.05 | 0.06 |
| S | 1650661 | Clostridium phoceensis | 6702 | 6702 | 0.05 | 0.06 |
| S | 1780376 | Alistipes sp. CHKCI003 | 6434 | 6434 | 0.05 | 0.06 |
| S | 1715004 | Clostridiales bacterium KLE1615 | 6268 | 6268 | 0.05 | 0.06 |
| S | 469614 | Erysipelotrichaceae bacterium 6_1 45 | 6245 | 6245 | 0.05 | 0.06 |
| S | 658089 | Lachnospiraceae bacterium 5_1_63FAA | 6232 | 6232 | 0.05 | 0.06 |
| S | 1965580 | Bacteroides sp. An19 | 5992 | 5992 | 0.04 | 0.06 |
| S | 552398 | Ruminococcaceae bacterium D16 | 5865 | 5865 | 0.04 | 0.05 |
| S | 1235803 | Parabacteroides sp. ASF519 | 5854 | 5854 | 0.04 | 0.05 |
| S | 1512 | [Clostridium] symbiosum | 5614 | 5614 | 0.04 | 0.05 |
| S | 457395 | Bacteroides sp. 9_1 42FAA | 5579 | 5579 | 0.04 | 0.05 |
| S | 1965576 | Pseudoflavonifractor sp. An184 | 5499 | 5499 | 0.04 | 0.05 |
| S | 1627893 | Ruminococcaceae bacterium cv2 | 5446 | 5446 | 0.04 | 0.05 |
| S | 28901 | Salmonella enterica | 5440 | 5440 | 0.04 | 0.05 |
| S | 563193 | Parabacteroides sp. D13 | 5371 | 5371 | 0.04 | 0.05 |
| S | 1520815 | Ruminococcaceae bacterium D5 | 5323 | 5323 | 0.04 | 0.05 |
| S | 1965604 | Anaeromassilibacillus sp. An250 | 5311 | 5311 | 0.04 | 0.05 |
| S | 1352 | Enterococcus faecium | 5235 | 5235 | 0.04 | 0.05 |
| S | 665941 | Coprobacillus sp. 3_3_56FAA | 5203 | 5203 | 0.04 | 0.05 |
| S | 457392 | Bacteroides sp. 3_2_5 | 5121 | 5121 | 0.04 | 0.05 |
| S | 626932 | Alistipes indistinctus | 5033 | 5033 | 0.04 | 0.05 |
| S | 1232439 | Clostridiales bacterium VE202-03 | 5005 | 5005 | 0.04 | 0.05 |
| S | 658087 | Lachnospiraceae bacterium 7_1_58FAA | 4994 | 4994 | 0.04 | 0.05 |
| S | 626930 | Bacteroides fluxus | 4981 | 4981 | 0.04 | 0.05 |
| S | 246787 | Bacteroides cellulosilyticus | 4967 | 4967 | 0.04 | 0.05 |
| S | 460384 | [Clostridium] lavalense | 4956 | 4956 | 0.04 | 0.05 |
| S | 1965587 | Anaeromassilibacillus sp. An200 | 4892 | 4892 | 0.04 | 0.05 |
| S | 469586 | Bacteroides sp. 1_1_6 | 4881 | 4881 | 0.04 | 0.05 |
| S | 338188 | Bacteroides finegoldii | 4870 | 4870 | 0.04 | 0.05 |
| S | 1870991 | Massilioclostridium coli | 4765 | 4765 | 0.03 | 0.04 |
| S | 1226322 | Oscillibacter sp. KLE 1728 | 4601 | 4601 | 0.03 | 0.04 |
| S | 1235797 | Oscillibacter sp. 1-3 | 4597 | 4597 | 0.03 | 0.04 |
| S | 1226323 | Oscillibacter sp. KLE 1745 | 4563 | 4563 | 0.03 | 0.04 |
| S | 106588 | Pseudoflavonifractor capillosus | 4523 | 4523 | 0.03 | 0.04 |
| S | 1965629 | Flavonifractor sp. An306 | 4516 | 4516 | 0.03 | 0.04 |
| S | 1806509 | Fusicatenibacter sp. 2789STDY5834925 | 4467 | 4467 | 0.03 | 0.04 |
| S | 411484 | Clostridium sp. SS2/1 | 4456 | 4456 | 0.03 | 0.04 |
| S | 1280669 | Dorea sp. AGR2135 | 4373 | 4373 | 0.03 | 0.04 |
| S | 742722 | Collinsella sp. 4_8_47FAA | 4358 | 4358 | 0.03 | 0.04 |
| S | 665950 | Lachnospiraceae bacterium 3_1_46FAA | 4356 | 4356 | 0.03 | 0.04 |
| S | 457387 | Bacteroides sp. 1_1_30 | 4310 | 4310 | 0.03 | 0.04 |
| S | 446660 | Adlercreutzia equolifaciens | 4293 | 4293 | 0.03 | 0.04 |
| S | 43306 | Pseudomonas denitrificans (nomen rejiciendum) | 4269 | 4269 | 0.03 | 0.04 |
| S | 1650663 | Fournierella massiliensis | 4216 | 4216 | 0.03 | 0.04 |
| S | 154046 | Hungatella hathewayi | 4197 | 4197 | 0.03 | 0.04 |
| S | 1739490 | Pseudomonas sp. HMSC073F05 | 4156 | 4156 | 0.03 | 0.04 |
| S | 469588 | Bacteroides sp. 2_1_22 | 4074 | 4074 | 0.03 | 0.04 |
| S | 1232445 | Clostridiales bacterium VE202-16 | 4034 | 4034 | 0.03 | 0.04 |
| S | 1965588 | Anaerofilum sp. An201 | 4013 | 4013 | 0.03 | 0.04 |
| S | 1522 | [Clostridium] innocuum | 3963 | 3963 | 0.03 | 0.04 |
| S | 1965537 | Flavonifractor sp. An10 | 3928 | 3928 | 0.03 | 0.04 |
| S | 1739298 | Bacteroides sp. HMSC067B03 | 3868 | 3868 | 0.03 | 0.04 |
| S | 39496 | Eubacterium ventriosum | 3840 | 3840 | 0.03 | 0.04 |
| S | 1673721 | Intestinimonas massiliensis | 3812 | 3812 | 0.03 | 0.04 |
| S | 61171 | Holdemania filiformis | 3796 | 3796 | 0.03 | 0.04 |
| S | 28111 | Bacteroides eggerthii | 3704 | 3704 | 0.03 | 0.03 |
| S | 1232459 | Oscillospiraceae bacterium VE202-24 | 3686 | 3686 | 0.03 | 0.03 |
| S | 411486 | Clostridium sp. M62/1 | 3656 | 3656 | 0.03 | 0.03 |
| S | 1232443 | Clostridiales bacterium VE202-13 | 3647 | 3647 | 0.03 | 0.03 |
| S | 1917876 | Blautia sp. Marseille-P3087 | 3613 | 3613 | 0.03 | 0.03 |
| S | 39483 | Faecalitalea cylindroides | 3601 | 3601 | 0.03 | 0.03 |
| S | 1720194 | Clostridium sp. AT4 | 3513 | 3513 | 0.03 | 0.03 |
| S | 649724 | Clostridium sp. ATCC BAA-442 | 3507 | 3507 | 0.03 | 0.03 |
| S | 301301 | Roseburia hominis | 3506 | 3506 | 0.03 | 0.03 |
| S | 329854 | Bacteroides intestinalis | 3505 | 3505 | 0.03 | 0.03 |
| S | 46228 | Ruminococcus lactaris | 3498 | 3498 | 0.03 | 0.03 |
| S | 693988 | Bilophila sp. 4_1_30 | 3462 | 3462 | 0.03 | 0.03 |
| S | 1871018 | Angelakisella massiliensis | 3451 | 3451 | 0.03 | 0.03 |
| S | 847 | Oxalobacter formigenes | 3417 | 3417 | 0.03 | 0.03 |
| S | 1465754 | Alistipes timonensis | 3358 | 3358 | 0.02 | 0.03 |
| S | 622 | Shigella dysenteriae | 3348 | 3348 | 0.02 | 0.03 |
| S | 1871015 | Pseudoflavonifractor sp. Marseille-P3106 | 3342 | 3342 | 0.02 | 0.03 |
| S | 1841868 | Provencibacterium massiliense | 3331 | 3331 | 0.02 | 0.03 |
| S | 457422 | Erysipelotrichaceae bacterium 2_2_44A | 3279 | 3279 | 0.02 | 0.03 |
| S | 908340 | Clostridium sp. HGF2 | 3252 | 3252 | 0.02 | 0.03 |
| S | 1965665 | Flavonifractor sp. An91 | 3238 | 3238 | 0.02 | 0.03 |
| S | 1308 | Streptococcus thermophilus | 3233 | 3233 | 0.02 | 0.03 |
| S | 1609975 | Clostridium sp. F541 | 3193 | 3193 | 0.02 | 0.03 |
| S | 457391 | Bacteroides sp. 3_1_33FAA | 3170 | 3170 | 0.02 | 0.03 |
| S | 1965664 | Flavonifractor sp. An9 | 3113 | 3113 | 0.02 | 0.03 |
| S | 84030 | [Clostridium] saccharolyticum | 3095 | 3095 | 0.02 | 0.03 |
| S | 384638 | [Bacteroides] pectinophilus | 3074 | 3074 | 0.02 | 0.03 |
| S | 1776382 | Neglecta timonensis | 3054 | 3054 | 0.02 | 0.03 |
| S | 28052 | Lachnospira pectinoschiza | 2966 | 2966 | 0.02 | 0.03 |
| S | 1965562 | Lachnoclostridium sp. An14 | 2943 | 2943 | 0.02 | 0.03 |
| S | 1965538 | Flavonifractor sp. An100 | 2929 | 2929 | 0.02 | 0.03 |
| S | 227866 | Bacillus tequilensis | 2921 | 2921 | 0.02 | 0.03 |
| S | 1739517 | Bacteroides sp. HMSC073E02 | 2895 | 2895 | 0.02 | 0.03 |
| S | 29347 | [Clostridium] scindens | 2877 | 2877 | 0.02 | 0.03 |
| S | 1280 | Staphylococcus aureus | 2834 | 2834 | 0.02 | 0.03 |
| S | 1965570 | Anaeromassilibacillus sp. An172 | 2804 | 2804 | 0.02 | 0.03 |
| S | 214851 | Subdoligranulum variabile | 2796 | 2796 | 0.02 | 0.03 |
| S | 626934 | Slackia piriformis | 2787 | 2787 | 0.02 | 0.03 |
| S | 2025877 | Parabacteroides sp. AT13 | 2749 | 2749 | 0.02 | 0.03 |
| S | 1871035 | Negativibacillus massiliensis | 2726 | 2726 | 0.02 | 0.03 |
| S | 1965548 | Drancourtella sp. An12 | 2675 | 2675 | 0.02 | 0.03 |
| S | 1965634 | Flavonifractor sp. An4 | 2660 | 2660 | 0.02 | 0.02 |
| S | 29361 | Tyzzerella nexilis | 2656 | 2656 | 0.02 | 0.02 |
| S | 457389 | Bacteroides sp. 3_1_13 | 2601 | 2601 | 0.02 | 0.02 |
| S | 457415 | Synergistes sp. 3_1_synl | 2585 | 2585 | 0.02 | 0.02 |
| S | 658086 | Lachnospiraceae bacterium 3_1_57FAA_CT1 | 2581 | 2581 | 0.02 | 0.02 |
| S | 574930 | Parabacteroides gordonii | 2560 | 2560 | 0.02 | 0.02 |
| S | 1816676 | Alistipes sp. Marseille-P2431 | 2547 | 2547 | 0.02 | 0.02 |
| S | 1855299 | Oscillibacter sp. PC13 | 2499 | 2499 | 0.02 | 0.02 |
| S | 2173 | Methanobrevibacter smithii | 2467 | 2467 | 0.02 | 0.02 |
| S | 1697787 | Clostridia bacterium UC5.1-1D10 | 2464 | 2464 | 0.02 | 0.02 |
| S | 508460 | Cloacibacillus evryensis | 2457 | 2457 | 0.02 | 0.02 |
| S | 621 | Shigella boydii | 2453 | 2453 | 0.02 | 0.02 |
| S | 1697788 | Clostridia bacterium UC5.1-2H6 | 2386 | 2386 | 0.02 | 0.02 |
| S | 1232453 | Clostridiales bacterium VE202-21 | 2379 | 2379 | 0.02 | 0.02 |
| S | 2018038 | Collinsella sp. TF06-26 | 2364 | 2364 | 0.02 | 0.02 |
| S | 1965660 | Flavonifractor sp. An82 | 2357 | 2357 | 0.02 | 0.02 |
| S | 1263547 | Oscillibacter ruminantium | 2342 | 2342 | 0.02 | 0.02 |
| S | 1944639 | Anaeromassilibacillus sp. Marseille-P3371 | 2316 | 2316 | 0.02 | 0.02 |
| S | 204516 | Bacteroides massiliensis | 2302 | 2302 | 0.02 | 0.02 |
| S | 1965542 | Eubacterium sp. Anil | 2285 | 2285 | 0.02 | 0.02 |
| S | 1965558 | Flavonifractor sp. An135 | 2279 | 2279 | 0.02 | 0.02 |
| S | 1871020 | Clostridium sp. Marseille-P3244 | 2266 | 2266 | 0.02 | 0.02 |
| S | 291644 | Bacteroides salyersiae | 2255 | 2255 | 0.02 | 0.02 |
| S | 1965581 | Faecalibacterium sp. An192 | 2254 | 2254 | 0.02 | 0.02 |
| S | 658655 | Lachnospiraceae bacterium 1_4_56FAA | 2236 | 2236 | 0.02 | 0.02 |
| S | 1265 | Ruminococcus flavefaciens | 2212 | 2212 | 0.02 | 0.02 |
| S | 341225 | [Clostridium] saccharogumia | 2177 | 2177 | 0.02 | 0.02 |
| S | 1965551 | Faecalibacterium sp. An122 | 2173 | 2173 | 0.02 | 0.02 |
| S | 469589 | Bacteroides sp. 2_1_33B | 2150 | 2150 | 0.02 | 0.02 |
| S | 342942 | [Clostridium] glycyrrhizinilyticum | 2095 | 2095 | 0.02 | 0.02 |
| S | 33043 | Coprococcus eutactus | 2071 | 2071 | 0.02 | 0.02 |
| S | 585544 | Bacteroides sp. D22 | 2065 | 2065 | 0.02 | 0.02 |
| S | 1750560 | Parabacteroides sp. SN4 | 2065 | 2065 | 0.02 | 0.02 |
| S | 358743 | [Clostridium] citroniae | 2003 | 2003 | 0.01 | 0.02 |
| S | 376806 | Bacteroides gallinarum | 1984 | 1984 | 0.01 | 0.02 |
| S | 1965661 | Pseudoflavonifractor sp. An85 | 1982 | 1982 | 0.01 | 0.02 |
| S | 1965639 | Gemmiger sp. An50 | 1926 | 1926 | 0.01 | 0.02 |
| S | 457390 | Bacteroides sp. 3_1_23 | 1886 | 1886 | 0.01 | 0.02 |
| S | 706562 | Lactobacillus rogosae | 1866 | 1866 | 0.01 | 0.02 |
| S | 626931 | Bacteroides oleiciplenus | 1849 | 1849 | 0.01 | 0.02 |
| S | 1965659 | Blautia sp. An81 | 1840 | 1840 | 0.01 | 0.02 |
| S | 1871030 | Merdibacter massiliensis | 1820 | 1820 | 0.01 | 0.02 |
| S | 1965549 | Gemmiger sp. An120 | 1734 | 1734 | 0.01 | 0.02 |
| S | 1378168 | Firmicutes bacterium ASF500 | 1711 | 1711 | 0.01 | 0.02 |
| S | 671267 | Bacteroides sartorii | 1666 | 1666 | 0.01 | 0.02 |
| S | 1965560 | Lachnoclostridium sp. An138 | 1660 | 1660 | 0.01 | 0.02 |
| S | 665940 | Clostridium sp. 7_3_54FAA | 1659 | 1659 | 0.01 | 0.02 |
| S | 351091 | Oscillibacter valericigenes | 1653 | 1653 | 0.01 | 0.02 |
| S | 40518 | Ruminococcus bromii | 1643 | 1643 | 0.01 | 0.02 |
| S | 1965632 | Bacteroides sp. An322 | 1640 | 1640 | 0.01 | 0.02 |
| S | 1468449 | Holdemania massiliensis | 1640 | 1640 | 0.01 | 0.02 |
| S | 165179 | Prevotella copri | 1630 | 1630 | 0.01 | 0.02 |
| S | 244366 | Klebsiella variicola | 1587 | 1587 | 0.01 | 0.01 |
| S | 31971 | [Eubacterium] dolichum | 1558 | 1558 | 0.01 | 0.01 |
| S | 1796613 | Bacteroides caecimuris | 1556 | 1556 | 0.01 | 0.01 |
| S | 1161942 | Ruminococcus champanellensis | 1524 | 1524 | 0.01 | 0.01 |
| S | 1965575 | Lachnoclostridium sp. An181 | 1501 | 1501 | 0.01 | 0.01 |
| S | 1965655 | Faecalibacterium sp. An77 | 1492 | 1492 | 0.01 | 0.01 |
| S | 1965635 | Pseudoflavonifractor sp. An44 | 1451 | 1451 | 0.01 | 0.01 |
| S | 290054 | Eubacterium coprostanoligenes | 1435 | 1435 | 0.01 | 0.01 |
| S | 1965571 | Erysipelatoclostridium sp. An173 | 1430 | 1430 | 0.01 | 0.01 |
| S | 1313 | Streptococcus pneumoniae | 1428 | 1428 | 0.01 | 0.01 |
| S | 53443 | Blautia hydrogenotrophica | 1416 | 1416 | 0.01 | 0.01 |
| S | 1720200 | Anaerotruncus rubiinfantis | 1378 | 1378 | 0.01 | 0.01 |
| S | 1805714 | Christensenella massiliensis | 1367 | 1367 | 0.01 | 0.01 |
| S | 1780381 | Eubacteriaceae bacterium CHKCI005 | 1349 | 1349 | 0.01 | 0.01 |
| S | 1720300 | Ruminococcus sp. AT10 | 1342 | 1342 | 0.01 | 0.01 |
| S | 624 | Shigella sonnei | 1334 | 1334 | 0.01 | 0.01 |
| S | 1965662 | Gemmiger sp. An87 | 1330 | 1330 | 0.01 | 0.01 |
| S | 45851 | Butyrivibrio crossotus | 1278 | 1278 | 0.01 | 0.01 |
| S | 1965650 | Alistipes sp. An66 | 1256 | 1256 | 0.01 | 0.01 |
| S | 550 | Enterobacter cloacae | 1256 | 1256 | 0.01 | 0.01 |
| S | 1335613 | Gordonibacter urolithinfaciens | 1239 | 1239 | 0.01 | 0.01 |
| S | 1965636 | Blautia sp. An46 | 1238 | 1238 | 0.01 | 0.01 |
| S | 1965603 | Blautia sp. An249 | 1231 | 1231 | 0.01 | 0.01 |
| S | 40519 | Ruminococcus callidus | 1213 | 1213 | 0.01 | 0.01 |
| S | 1232460 | Clostridiales bacterium VE202-28 | 1194 | 1194 | 0.01 | 0.01 |
| S | 471189 | Gordonibacter pamelaeae | 1193 | 1193 | 0.01 | 0.01 |
| S | 1679169 | Bacillus sp. FJAT-27916 | 1192 | 1192 | 0.01 | 0.01 |
| S | 158836 | Enterobacter hormaechei | 1191 | 1191 | 0.01 | 0.01 |
| S | 44749 | Sporobacter termitidis | 1181 | 1181 | 0.01 | 0.01 |
| S | 1871021 | Lachnoclostridium phocaeense | 1176 | 1176 | 0.01 | 0.01 |
| S | 1322 | Blautia hansenii | 1154 | 1154 | 0.01 | 0.01 |
| S | 712118 | Actinomyces sp. oral taxon 172 | 1149 | 1149 | 0.01 | 0.01 |
| S | 626940 | Phascolarctobacterium succinatutens | 1139 | 1139 | 0.01 | 0.01 |
| S | 290053 | Bacteroides helcogenes | 1135 | 1135 | 0.01 | 0.01 |
| S | 84112 | Eggerthella lenta | 1120 | 1120 | 0.01 | 0.01 |
| S | 1226325 | Clostridium sp. KLE 1755 | 1119 | 1119 | 0.01 | 0.01 |
| S | 28139 | Rikenella microfusus | 1091 | 1091 | 0.01 | 0.01 |
| S | 1492 | Clostridium butyricum | 1088 | 1088 | 0.01 | 0.01 |
| S | 1965578 | Pseudoflavonifractor sp. An187 | 1071 | 1071 | 0.01 | 0.01 |
| S | 180164 | Blautia schinkii | 1068 | 1068 | 0.01 | 0.01 |
| S | 1965666 | Flavonifractor sp. An92 | 1066 | 1066 | 0.01 | 0.01 |
| S | 1987501 | Massilimaliae sp. Marseille-P3753 | 1057 | 1057 | 0.01 | 0.01 |
| S | 556259 | Bacteroides sp. D2 | 1053 | 1053 | 0.01 | 0.01 |
| S | 469587 | Bacteroides sp. 2_1_16 | 1052 | 1052 | 0.01 | 0.01 |
| S | 1965654 | Lachnoclostridium sp. An76 | 1048 | 1048 | 0.01 | 0.01 |
| S | 1965648 | Faecalibacterium sp. An58 | 1039 | 1039 | 0.01 | 0.01 |
| S | 1408819 | Youngiibacter fragilis | 1037 | 1037 | 0.01 | 0.01 |
| S | 1358 | Lactococcus lactis | 1036 | 1036 | 0.01 | 0.01 |
| S | 1870993 | Tyzzerella sp. Marseille-P3062 | 1029 | 1029 | 0.01 | 0.01 |
| S | 39484 | Butyricicoccus desmolans | 1025 | 1025 | 0.01 | 0.01 |
| S | 1965569 | Lachnoclostridium sp. An169 | 1024 | 1024 | 0.01 | 0.01 |
| S | 33035 | Blautia producta | 1020 | 1020 | 0.01 | 0.01 |
| S | 1965572 | Pseudoflavonifractor sp. An176 | 1020 | 1020 | 0.01 | 0.01 |
| S | 1780379 | Clostridiales bacterium CHKCI006 | 1011 | 1011 | 0.01 | 0.01 |
| S | 1776384 | Emergencia timonensis | 999 | 999 | 0.01 | 0.01 |
| S | 1898207 | Clostridiales bacterium | 985 | 985 | 0.01 | 0.01 |
| S | 1703332 | Lachnospiraceae bacterium TF01-11 | 977 | 977 | 0.01 | 0.01 |
| S | 1200751 | Ruminococcaceae bacterium P7 | 974 | 974 | 0.01 | 0.01 |
| S | 1653435 | Merdimonas faecis | 969 | 969 | 0.01 | 0.01 |
| S | 877414 | Clostridiales bacterium NK3B98 | 969 | 969 | 0.01 | 0.01 |
| S | 1697794 | Clostridia bacterium UC5.1-1D1 | 969 | 969 | 0.01 | 0.01 |
| S | 1078087 | Parabacteroides sp. HG50025 | 967 | 967 | 0.01 | 0.01 |
| S | 467210 | Lachnoanaerobaculum saburreum | 962 | 962 | 0.01 | 0.01 |
| S | 1903262 | Bacteroides sp. Marseille-P3108 | 957 | 957 | 0.01 | 0.01 |
| S | 1852384 | Massilimaliae massiliensis | 951 | 951 | 0.01 | 0.01 |
| S | 674529 | Bacteroides faecis | 940 | 940 | 0.01 | 0.01 |
| S | 1965583 | Lachnoclostridium sp. An196 | 927 | 927 | 0.01 | 0.01 |
| S | 1965642 | Flavonifractor sp. An52 | 926 | 926 | 0.01 | 0.01 |
| S | 1264 | Ruminococcus albus | 909 | 909 | 0.01 | 0.01 |
| S | 216816 | Bifidobacterium longum | 908 | 908 | 0.01 | 0.01 |
| S | 1739319 | Bacteroides sp. HMSC068A09 | 899 | 899 | 0.01 | 0.01 |
| S | 1673717 | Anaeromassilibacillus senegalensis | 896 | 896 | 0.01 | 0.01 |
| S | 1841855 | Bacteroides sp. Marseille-P2653 | 881 | 881 | 0.01 | 0.01 |
| S | 97253 | Eubacterium plexicaudatum | 869 | 869 | 0.01 | 0.01 |
| S | 658088 | Lachnospiraceae bacterium 9_1_43BFAA | 867 | 867 | 0.01 | 0.01 |
| S | 1965546 | Alistipes sp. An116 | 863 | 863 | 0.01 | 0.01 |
| S | 1965640 | Bacteroides sp. An51A | 852 | 852 | 0.01 | 0.01 |
| S | 1549 | [Clostridium] sporosphaeroides | 852 | 852 | 0.01 | 0.01 |
| S | 469597 | Coprobacillus sp. 8_2_54BFAA | 851 | 851 | 0.01 | 0.01 |
| S | 1261634 | Roseburia sp. 499 | 848 | 848 | 0.01 | 0.01 |
| S | 1965627 | Lachnoclostridium sp. An298 | 848 | 848 | 0.01 | 0.01 |
| S | 1965555 | Lachnoclostridium sp. An131 | 834 | 834 | 0.01 | 0.01 |
| S | 1120746 | bacterium MS4 | 828 | 828 | 0.01 | 0.01 |
| S | 871324 | Bacteroides stercorirosoris | 826 | 826 | 0.01 | 0.01 |
| S | 1965573 | Drancourtella sp. An177 | 819 | 819 | 0.01 | 0.01 |
| S | 1917883 | Bacteroides sp. Marseille-P3166 | 804 | 804 | 0.01 | 0.01 |
| S | 1470345 | Bacteroides timonensis | 794 | 794 | 0.01 | 0.01 |
| S | 655809 | Prevotella sp. oral taxon 472 | 788 | 788 | 0.01 | 0.01 |
| S | 1965550 | Faecalibacterium sp. An121 | 786 | 786 | 0.01 | 0.01 |
| S | 548 | Klebsiella aerogenes | 781 | 781 | 0.01 | 0.01 |
| S | 168384 | Marvinbryantia formatexigens | 778 | 778 | 0.01 | 0.01 |
| S | 1780380 | Eubacteriaceae bacterium CHKCI004 | 778 | 778 | 0.01 | 0.01 |
| S | 1965544 | Flavonifractor sp. An112 | 775 | 775 | 0.01 | 0.01 |
| S | 1720313 | Bittarella massiliensis | 766 | 766 | 0.01 | 0.01 |
| S | 1965586 | Mediterranea sp. An20 | 762 | 762 | 0.01 | 0.01 |
| S | 1287488 | Prevotella sp. S7 MS 2 | 752 | 752 | 0.01 | 0.01 |
| S | 1773 | Mycobacterium tuberculosis | 750 | 750 | 0.01 | 0.01 |
| S | 1501392 | Coprobacter secundus | 743 | 743 | 0.01 | 0.01 |
| S | 1232444 | Clostridiales bacterium VE202-15 | 743 | 743 | 0.01 | 0.01 |
| S | 1965613 | Bacteroides sp. An269 | 742 | 742 | 0.01 | 0.01 |
| S | 376805 | Bacteroides salanitronis | 740 | 740 | 0.01 | 0.01 |
| S | 1965645 | Alistipes sp. An54 | 738 | 738 | 0.01 | 0.01 |
| S | 258515 | Acetanaerobacterium elongatum | 726 | 726 | 0.01 | 0.01 |
| S | 1871029 | Intestinibacillus massiliensis | 724 | 724 | 0.01 | 0.01 |
| S | 270498 | Catabacter hongkongensis | 721 | 721 | 0.01 | 0.01 |
| S | 1852361 | Actinomyces sp. Marseille-P2825 | 717 | 717 | 0.01 | 0.01 |
| S | 971 | Selenomonas ruminantium | 715 | 715 | 0.01 | 0.01 |
| S | 333367 | [Clostridium] asparagiforme | 709 | 709 | 0.01 | 0.01 |
| S | 1965631 | Alistipes sp. An31A | 707 | 707 | 0.01 | 0.01 |
| S | 1235835 | Anaerotruncus sp. G3(2012) | 697 | 697 | 0.01 | 0.01 |
| S | 1491 | Clostridium botulinum | 694 | 694 | 0.01 | 0.01 |
| S | 397288 | Lachnospiraceae bacterium 3-1 | 691 | 691 | 0.01 | 0.01 |
| S | 100176 | Papillibacter cinnamivorans | 687 | 687 | 0.01 | 0.01 |
| S | 1470354 | Candidatus Soleaferrea massiliensis | 682 | 682 | 0 | 0.01 |
| S | 1623 | Lactobacillus ruminis | 682 | 682 | 0 | 0.01 |
| S | 310300 | Bacteroides pyogenes | 672 | 672 | 0 | 0.01 |
| S | 1776391 | Romboutsia timonensis | 661 | 661 | 0 | 0.01 |
| S | 1907659 | Blautia sp. Marseille-P3201T | 660 | 660 | 0 | 0.01 |
| S | 1965628 | Eubacterium sp. An3 | 654 | 654 | 0 | 0.01 |
| S | 1871013 | Parabacteroides sp. Marseille-P3236 | 653 | 653 | 0 | 0.01 |
| S | 290052 | Acetivibrio ethanolgignens | 652 | 652 | 0 | 0.01 |
| S | 1965589 | Drancourtella sp. An210 | 648 | 648 | 0 | 0.01 |
| S | 936595 | Lachnoanaerobaculum sp. OBRCS-5 | 644 | 644 | 0 | 0.01 |
| S | 39482 | Faecalicatena contorta | 641 | 641 | 0 | 0.01 |
| S | 1078090 | Coprococcus sp. HPP0074 | 636 | 636 | 0 | 0.01 |
| S | 1235800 | Lachnospiraceae bacterium 10-1 | 629 | 629 | 0 | 0.01 |
| S | 93974 | Bacteroides sp. AR20 | 628 | 628 | 0 | 0.01 |
| S | 1852383 | Ruminococcaceae bacterium Marseille-P2935 | 625 | 625 | 0 | 0.01 |
| S | 1351 | Enterococcus faecalis | 622 | 622 | 0 | 0.01 |
| S | 1945634 | Butyricicoccus sp. BB10 | 615 | 615 | 0 | 0.01 |
| S | 208962 | Escherichia albertii | 612 | 612 | 0 | 0.01 |
| S | 253239 | Ethanoligenens harbinense | 609 | 609 | 0 | 0.01 |
| S | 1676614 | Prevotella sp. 109 | 608 | 608 | 0 | 0.01 |
| S | 470 | Acinetobacter baumannii | 605 | 605 | 0 | 0.01 |
| S | 1352374 | Ruminococcus sp. YE78 | 604 | 604 | 0 | 0.01 |
| S | 84026 | [Clostridium] methylpentosum | 602 | 602 | 0 | 0.01 |
| S | 556270 | Coprobacillus sp. D7 | 593 | 593 | 0 | 0.01 |
| S | 1965582 | Gemmiger sp. An194 | 591 | 591 | 0 | 0.01 |
| S | 1849176 | Clostridium sp. W14A | 582 | 582 | 0 | 0.01 |
| S | 387090 | Bacteroides coprophilus | 581 | 581 | 0 | 0.01 |
| S | 1871003 | Tidjanibacter massiliensis | 575 | 575 | 0 | 0.01 |
| S | 411489 | Clostridium sp. L2-50 | 573 | 573 | 0 | 0.01 |
| S | 454155 | Paraprevotella xylaniphila | 572 | 572 | 0 | 0.01 |
| S | 615 | Serratia marcescens | 563 | 563 | 0 | 0.01 |
| S | 1235799 | Lachnospiraceae bacterium 3-2 | 556 | 556 | 0 | 0.01 |
| S | 147802 | Lactobacillus iners | 555 | 555 | 0 | 0.01 |
| S | 1235798 | Dorea sp. 5-2 | 546 | 546 | 0 | 0.01 |
| S | 1841865 | Mediterranea massiliensis | 539 | 539 | 0 | 0.01 |
| S | 1519438 | Eubacterium sp. ER2 | 533 | 533 | 0 | 0 |
| S | 97139 | Clostridium sp. ASF502 | 521 | 521 | 0 | 0 |
| S | 1581118 | Pseudomonas sp. HMSCO5H02 | 506 | 506 | 0 | 0 |
| S | 1261635 | Roseburia sp. 831b | 505 | 505 | 0 | 0 |
| S | 1547597 | Sanguibacteroides justesenii | 499 | 499 | 0 | 0 |
| S | 303 | Pseudomonas putida | 494 | 494 | 0 | 0 |
| S | 1841867 | Phocea massiliensis | 489 | 489 | 0 | 0 |
| S | 457421 | Clostridiales bacterium 1_7_47FAA | 488 | 488 | 0 | 0 |
| S | 397287 | Lachnospiraceae bacterium 28-4 | 484 | 484 | 0 | 0 |
| S | 1841856 | Bacteroides mediterraneensis | 476 | 476 | 0 | 0 |
| S | 658656 | Lachnospiraceae bacterium 6_1_37FAA | 472 | 472 | 0 | 0 |
| S | 397290 | Lachnospiraceae bacterium A2 | 471 | 471 | 0 | 0 |
| S | 143393 | [Eubacterium] sulci | 469 | 469 | 0 | 0 |
| S | 1235790 | Eubacterium sp. 14-2 | 465 | 465 | 0 | 0 |
| S | 1160721 | Ruminococcus bicirculans | 460 | 460 | 0 | 0 |
| S | 180311 | Hespellia stercorisuis | 455 | 455 | 0 | 0 |
| S | 1658109 | Candidatus Stoquefichus sp. SB1 | 453 | 453 | 0 | 0 |
| S | 1261637 | Anaerostipes sp. 992a | 450 | 450 | 0 | 0 |
| S | 1697793 | Clostridia bacterium UC5.1-1E11 | 450 | 450 | 0 | 0 |
| S | 1602172 | Prevotella sp. P5-125 | 449 | 449 | 0 | 0 |
| S | 39497 | Eubacterium xylanophilum | 444 | 444 | 0 | 0 |
| S | 1776379 | Prevotella sp. KHD1 | 441 | 441 | 0 | 0 |
| S | 1911 | Streptomyces griseus | 441 | 441 | 0 | 0 |
| S | 1739250 | Pseudomonas sp. HMSC075A08 | 434 | 434 | 0 | 0 |
| S | 1888 | Streptomyces albus | 429 | 429 | 0 | 0 |
| S | 1235793 | Lachnospiraceae bacterium COE1 | 428 | 428 | 0 | 0 |
| S | 13690 | Sphingobium yanoikuyae | 428 | 428 | 0 | 0 |
| S | 376804 | Bacteroides barnesiae | 423 | 423 | 0 | 0 |
| S | 397291 | Lachnospiraceae bacterium A4 | 423 | 423 | 0 | 0 |
| S | 310297 | Bacteroides plebeius | 421 | 421 | 0 | 0 |
| S | 294 | Pseudomonas fluorescens | 420 | 420 | 0 | 0 |
| S | 1347393 | Bacteroides neonati | 416 | 416 | 0 | 0 |
| S | 1163670 | Bacteroides sp. 14(A) | 413 | 413 | 0 | 0 |
| S | 544645 | Butyricimonas virosa | 412 | 412 | 0 | 0 |
| S | 1526 | [Clostridium] aminophilum | 406 | 406 | 0 | 0 |
| S | 1852366 | Holdemania sp. Marseille-P2844 | 403 | 403 | 0 | 0 |
| S | 1780378 | Clostridiales bacterium CHKCI001 | 402 | 402 | 0 | 0 |
| S | 1572656 | Ruminococcaceae bacterium CPB6 | 401 | 401 | 0 | 0 |
| S | 53442 | Eubacterium callanderi | 401 | 401 | 0 | 0 |
| S | 742723 | Lachnospiraceae bacterium 2_1_46FAA | 394 | 394 | 0 | 0 |
| S | 552396 | Erysipelotrichaceae bacterium 5_2_54FAA | 391 | 391 | 0 | 0 |
| S | 1871006 | Bacteroides sp. Marseille-P3132 | 387 | 387 | 0 | 0 |
| S | 1540257 | Clostridium sp. KNHs214 | 387 | 387 | 0 | 0 |
| S | 1566277 | Paenibacillus sp. 276b | 385 | 385 | 0 | 0 |
| S | 29343 | [Clostridium] cellulosi | 384 | 384 | 0 | 0 |
| S | 454154 | Paraprevotella clara | 383 | 383 | 0 | 0 |
| S | 1965547 | Lachnoclostridium sp. An118 | 380 | 380 | 0 | 0 |
| S | 1903263 | Traorella massiliensis | 375 | 375 | 0 | 0 |
| S | 910311 | Eggerthella sp. HGA1 | 373 | 373 | 0 | 0 |
| S | 1520817 | Ruminococcaceae bacterium FB2012 | 369 | 369 | 0 | 0 |
| S | 341220 | Lactonifactor longoviformis | 369 | 369 | 0 | 0 |
| S | 1134687 | Klebsiella michiganensis | 369 | 369 | 0 | 0 |
| S | 1745713 | Bariatricus massiliensis | 366 | 366 | 0 | 0 |
| S | 1958780 | Clostridium merdae | 366 | 366 | 0 | 0 |
| S | 1182695 | Klebsiella sp. KTE92 | 364 | 364 | 0 | 0 |
| S | 85831 | Bacteroides acidifaciens | 358 | 358 | 0 | 0 |
| S | 1235792 | Lachnospiraceae bacterium M18-1 | 358 | 358 | 0 | 0 |
| S | 438033 | Ruminococcus gauvreauii | 352 | 352 | 0 | 0 |
| S | 1653434 | Sellimonas intestinalis | 351 | 351 | 0 | 0 |
| S | 1686379 | Citrobacter sp. MGH104 | 350 | 350 | 0 | 0 |
| S | 604330 | Olsenella umbonata | 348 | 348 | 0 | 0 |
| S | 1458462 | Lachnospiraceae bacterium AC2029 | 344 | 344 | 0 | 0 |
| S | 2025494 | Anaerostipes sp. BG01 | 344 | 344 | 0 | 0 |
| S | 796942 | Stomatobaculum longum | 344 | 344 | 0 | 0 |
| S | 40841 | Succiniclasticum ruminis | 344 | 344 | 0 | 0 |
| S | 1261636 | Anaerostipes sp. 494a | 342 | 342 | 0 | 0 |
| S | 665943 | Eggerthella sp. 1_3_56FAA | 341 | 341 | 0 | 0 |
| S | 474960 | Hydrogenoanaerobacterium saccharovorans | 340 | 340 | 0 | 0 |
| S | 658085 | Lachnospiraceae bacterium 5_1_57FAA | 338 | 338 | 0 | 0 |
| S | 487174 | Barnesiella intestinihominis | 335 | 335 | 0 | 0 |
| S | 729 | Haemophilus parainfluenzae | 333 | 333 | 0 | 0 |

TABLE 8
Report of the Abundance of Archaea

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| D | 2157 Archaea | 12705 | 0 | 0.09 | 0.12 |
| S | 1406512 Candidatus | 9667 | 9667 | 0.07 | 0.09 |
| | Methanomassiliicoccus intestinalis | | | | |
| S | 2173 Methanobrevibacter smithii | 2467 | 2467 | 0.02 | 0.02 |
| S | 2187 Methanococcus vannielii | 21 | 21 | 0 | 0 |
| S | 1080712 Methanomassiliicoccus | 14 | 14 | 0 | 0 |
| | luminyensis | | | | |
| | 49547 Methanobrevibacter curvatus | 13 | 13 | 0 | 0 |
| | 39152 Methanococcus maripaludis | 12 | 12 | 0 | 0 |
| | 2223 Methanosaeta concilii | 9 | 9 | 0 | 0 |
| S | 1898749 Candidatus Nitrosomarinus | 9 | 9 | 0 | 0 |
| | catalina | | | | |
| S | 39441 Methanobrevibacter arboriphilus | 8 | 8 | 0 | 0 |
| | 660521 Halogranum gelatinilyticum | 8 | 8 | 0 | 0 |
| S | 332168 Halococcus hamelinensis | 8 | 8 | 0 | 0 |
| S | 1495314 Methanoculleus sp. MH98A | 8 | 8 | 0 | 0 |
| | 83984 Methanocorpusculum Iabreanum | 8 | 8 | 0 | 0 |
| S | 1561964 Methanosphaera sp. WGK6 | 7 | 7 | 0 | 0 |
| | 43928 Halobaculum gomorrense | 7 | 7 | 0 | 0 |
| | 413812 Natronococcus jeotgali | 7 | 7 | 0 | 0 |
| | 54076 Thermococcus zilligii | 7 | 7 | 0 | 0 |
| | 2201 Methanofollis liminatans | 6 | 6 | 0 | 0 |
| | 71518 Methanocorpusculum bavaricum | 6 | 6 | 0 | 0 |
| | 418008 Methanosarcina horonobensis | 6 | 6 | 0 | 0 |
| S | 1175445 Methanocella arvoryzae | 6 | 6 | 0 | 0 |
| | 644281 Methanocaldococcus sp. | 6 | 6 | 0 | 0 |
| | FS406-22 | | | | |
| | 294671 Methanobrevibacter olleyae | 5 | 5 | 0 | 0 |
| S | 1379702 Methanobacterium sp. MB1 | 5 | 5 | 0 | 0 |
| S | 1380430 Halolamina rubra | 5 | 5 | 0 | 0 |
| S | 1679489 Haladaptatus sp. R4 | 5 | 5 | 0 | 0 |
| | 301967 Haloterrigena saccharevitans | 5 | 5 | 0 | 0 |
| | 253108 Natronolimnobius baerhuensis | 5 | 5 | 0 | 0 |
| | 118126 Methanoculleus chikugoensis | 5 | 5 | 0 | 0 |
| | 420950 Methanolobus psychrophilus | 5 | 5 | 0 | 0 |
| | 301375 Methanosaeta harundinacea | 5 | 5 | 0 | 0 |
| S | 1392998 Candidatus Methanoperedens | 5 | 5 | 0 | 0 |
| | nitroreducens | | | | |
| | 190974 Methanobrevibacter | 4 | 4 | 0 | 0 |
| | gottschalkii | | | | |
| | 66851 Methanobrevibacter oralis | 4 | 4 | 0 | 0 |
| | 145261 Methanothermobacter wolfeii | 4 | 4 | 0 | 0 |
| S | 1514971 Haloprofundus marisrubri | 4 | 4 | 0 | 0 |
| S | 1604004 Halanaeroarchaeum | 4 | 4 | 0 | 0 |
| | sulfurireducens | | | | |
| S | 1906246 Natronomonas sp. CBA1134 | 4 | 4 | 0 | 0 |
| S | 2008440 Thermococcus sp. 5-4 | 4 | 4 | 0 | 0 |
| S | 1609968 Methanobrevibacter sp. | 3 | 3 | 0 | 0 |
| | YE315 | | | | |
| S | 1495336 Methanobacterium sp. SMA-27 | 3 | 3 | 0 | 0 |
| S | 1853690 Halolamina sp. CBA1230 | 3 | 3 | 0 | 0 |
| S | 376171 Haloplanus natans | 3 | 3 | 0 | 0 |
| S | 1236180 Halopelagius longus | 3 | 3 | 0 | 0 |
| S | 2242 Halobacterium salinarum | 3 | 3 | 0 | 0 |
| S | 767519 Halomicrobium zhouii | 3 | 3 | 0 | 0 |
| S | 13769 Natrialba magadii | 3 | 3 | 0 | 0 |
| S | 528314 Methanoculleus horonobensis | 3 | 3 | 0 | 0 |
| S | 38027 Methanosarcina siciliae | 3 | 3 | 0 | 0 |
| S | 38026 Methanolobus vulcani | 3 | 3 | 0 | 0 |
| S | 1175444 Methanocella conradii | 3 | 3 | 0 | 0 |
| S | 187878 Thermococcus gammatolerans | 3 | 3 | 0 | 0 |
| S | 43687 Metallosphaera sedula | 3 | 3 | 0 | 0 |
| S | 97393 Ferroplasma acidarmanus | 2 | 2 | 0 | 0 |
| S | 1860099 Methanobrevibacter sp. A27 | 2 | 2 | 0 | 0 |
| S | 230361 Methanobrevibacter millerae | 2 | 2 | 0 | 0 |
| S | 190977 Methanobrevibacter wolinii | 2 | 2 | 0 | 0 |
| S | 83816 Methanobrevibacter | 2 | 2 | 0 | 0 |
| | ruminantium | | | | |
| S | 145262 Methanothermobacter | 2 | 2 | 0 | 0 |
| | thermautotrophicus | | | | |
| S | 1419722 Halorubrum sp. SD626R | 2 | 2 | 0 | 0 |
| S | 2247 Halorubrum lacusprofundi | 2 | 2 | 0 | 0 |
| S | 553469 Halogeometricum rufum | 2 | 2 | 0 | 0 |
| S | 553468 Haladaptatus litoreus | 2 | 2 | 0 | 0 |
| S | 1407499 Halobacterium hubeiense | 2 | 2 | 0 | 0 |
| S | 996166 Haloarchaeobius iranensis | 2 | 2 | 0 | 0 |
| S | 29282 Haloarcula japonica | 2 | 2 | 0 | 0 |
| S | 146826 Halorhabdus utahensis | 2 | 2 | 0 | 0 |
| S | 392421 Haloterrigena hispanica | 2 | 2 | 0 | 0 |
| S | 1202768 Halopiger salifodinae | 2 | 2 | 0 | 0 |
| S | 68911 Natrialba chahannaoensis | 2 | 2 | 0 | 0 |
| S | 1095778 Natronobacterium | 2 | 2 | 0 | 0 |
| | texcoconense | | | | |
| S | 83986 Methanoculleus bourgensis | 2 | 2 | 0 | 0 |
| S | 358766 Methanoregula boonei | 2 | 2 | 0 | 0 |
| S | 73913 Methanocaldococcus vulcanius | 2 | 2 | 0 | 0 |
| S | 1042877 Thermococcus sp. 4557 | 2 | 2 | 0 | 0 |
| S | 582419 Thermococcus paralvinellae | 2 | 2 | 0 | 0 |
| S | 2234 Archaeoglobus fulgidus | 2 | 2 | 0 | 0 |
| S | 1034015 Nitrososphaera viennensis | 2 | 2 | 0 | 0 |
| S | 756883 halophilic archaeon DL31 | 2 | 2 | 0 | 0 |
| S | 1769296 Candidatus | 1 | 1 | 0 | 0 |
| | Methanomethylophilus sp. 1R26 | | | | |
| S | 2303 Thermoplasma acidophilum | 1 | 1 | 0 | 0 |
| S | 1495144 methanogenic archaeon IS04-H5 | 1 | 1 | 0 | 0 |
| S | 387957 Methanobrevibacter sp. 87.7 | 1 | 1 | 0 | 0 |
| S | 55758 Methanobrevibacter filiformis | 1 | 1 | 0 | 0 |
| S | 47311 Methanobrevibacter cuticularis | 1 | 1 | 0 | 0 |
| S | 877455 Methanobacterium lacus | 1 | 1 | 0 | 0 |
| S | 868131 Methanobacterium paludis | 1 | 1 | 0 | 0 |
| S | 2162 Methanobacterium formicicum | 1 | 1 | 0 | 0 |
| S | 1765655 Halorubrum tropicale | 1 | 1 | 0 | 0 |
| S | 1383851 Halorubrum halodurans | 1 | 1 | 0 | 0 |
| S | 1227480 Halorubrum hochstenium | 1 | 1 | 0 | 0 |
| S | 1194088 Halorubrum sp. T3 | 1 | 1 | 0 | 0 |
| | 368623 Halorubrum aidingense | 1 | 1 | 0 | 0 |
| | 35743 Halorubrum sodomense | 1 | 1 | 0 | 0 |
| | 2248 Halorubrum saccharovorum | 1 | 1 | 0 | 0 |
| S | 1267564 Halopenitus malekzadehii | 1 | 1 | 0 | 0 |
| S | 1048396 Halopenitus persicus | 1 | 1 | 0 | 0 |
| | 693851 Halogranum salarium | 1 | 1 | 0 | 0 |
| | 403181 Haloferax mucosum | 1 | 1 | 0 | 0 |
| | 555874 Haloplanus vescus | 1 | 1 | 0 | 0 |
| | 411361 Halogeometricum pallidum | 1 | 1 | 0 | 0 |
| | 453847 Haladaptatus cibarius | 1 | 1 | 0 | 0 |
| | 367189 Haladaptatus paucihalophilus | 1 | 1 | 0 | 0 |
| | 751944 Halobacterium sp. DL1 | 1 | 1 | 0 | 0 |
| S | 1008153 Halalkalicoccus | 1 | 1 | 0 | 0 |
| | paucihalophilus | | | | |
| | 2257 Natronomonas pharaonis | 1 | 1 | 0 | 0 |
| S | 1457250 Halapricum salinum | 1 | 1 | 0 | 0 |
| | 660518 Halorientalis regularis | 1 | 1 | 0 | 0 |
| | 171164 Halosimplex carlsbadense | 1 | 1 | 0 | 0 |
| S | 1264579 Halococcus sediminicola | 1 | 1 | 0 | 0 |
| S | 1232219 Halococcus agarilyticus | 1 | 1 | 0 | 0 |
| | 335952 Halococcus thailandensis | 1 | 1 | 0 | 0 |
| | 504937 Haloterrigena salina | 1 | 1 | 0 | 0 |
| | 370323 Haloterrigena limicola | 1 | 1 | 0 | 0 |
| | 253107 Natronolimnobius | 1 | 1 | 0 | 0 |
| | innermongolicus | | | | |
| S | 1293048 Halopiger goleimassiliensis | 1 | 1 | 0 | 0 |
| S | 1293047 Halopiger djelfimassiliensis | 1 | 1 | 0 | 0 |
| | 387343 Halopiger xanaduensis | 1 | 1 | 0 | 0 |
| | 44930 Natronobacterium gregoryi | 1 | 1 | 0 | 0 |
| S | 1095776 Natronorubrum texcoconense | 1 | 1 | 0 | 0 |
| | 63128 Natronorubrum tibetense | 1 | 1 | 0 | 0 |
| | 148448 Halobiforma haloterrestris | 1 | 1 | 0 | 0 |
| | 54120 Methanolacinia petrolearia | 1 | 1 | 0 | 0 |
| | 2315 Methanoplanus limicola | 1 | 1 | 0 | 0 |
| | 2205 Methanomicrobium mobile | 1 | 1 | 0 | 0 |
| | 2197 Methanogenium cariaci | 1 | 1 | 0 | 0 |
| | 882104 Methanoregula formicica | 1 | 1 | 0 | 0 |
| | 263906 Methanolinea tarda | 1 | 1 | 0 | 0 |
| S | 2203 Methanospirillum hungatei | 1 | 1 | 0 | 0 |
| S | 1483602 Methanosarcina sp. | 1 | 1 | 0 | 0 |
| | 1.H.T.1A.1 | | | | |
| S | 1434102 Methanosarcina sp. WH1 | 1 | 1 | 0 | 0 |
| S | 2208 Methanosarcina barkeri | 1 | 1 | 0 | 0 |
| S | 39669 Methanosalsum zhilinae | 1 | 1 | 0 | 0 |
| S | 101192 Methanomethylovorans | 1 | 1 | 0 | 0 |
| | hollandica | | | | |
| S | 39664 Methanohalophilus portucalensis | 1 | 1 | 0 | 0 |
| S | 2188 Methanococcus voltae | 1 | 1 | 0 | 0 |
| S | 83171 Methanocaldococcus fervens | 1 | 1 | 0 | 0 |
| S | 195522 Thermococcus nautili | 1 | 1 | 0 | 0 |
| S | 172049 Thermococcus sibiricus | 1 | 1 | 0 | 0 |
| S | 122420 Thermococcus sp. P6 | 1 | 1 | 0 | 0 |
| S | 72803 Thermococcus siculi | 1 | 1 | 0 | 0 |
| S | 55802 Thermococcus barophilus | 1 | 1 | 0 | 0 |
| S | 53952 Thermococcus peptonophilus | 1 | 1 | 0 | 0 |
| S | 49899 Thermococcus profundus | 1 | 1 | 0 | 0 |
| S | 1008460 Pyrococcus yayanosii | 1 | 1 | 0 | 0 |
| S | 565033 Geoglobus acetivorans | 1 | 1 | 0 | 0 |
| S | 160233 Ignicoccus hospitalis | 1 | 1 | 0 | 0 |
| S | 84599 Staphylothermus hellenicus | 1 | 1 | 0 | 0 |
| S | 1650660 Vulcanisaeta sp. EB80 | 1 | 1 | 0 | 0 |

TABLE 9
Report of the Abundance of Eukaryotes

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| z | 2759 Eukaryota | 1701 | 0 | 0.01 | 0.02 |
| D1 | 33154 Opisthokonta | 707 | 0 | 0.01 | 0.01 |
| K | 4751 Fungi | 707 | 0 | 0.01 | 0.01 |
| K1 | 451864 Dikarya | 621 | 0 | 0 | 0.01 |
| P | 4890 Ascomycota | 547 | 0 | 0 | 0.01 |
| P1 | 716545 saccharomyceta | 545 | 0 | 0 | 0.01 |
| P2 | 147538 Pezizomycotina | 407 | 0 | 0 | 0 |
| P3 | 716546 leotiomyceta | 397 | 0 | 0 | 0 |
| P4 | 715989 sordariomyceta | 173 | 0 | 0 | 0 |
| C | 147550 Sordariomycetes | 145 | 0 | 0 | 0 |
| C1 | 222543 | 89 | 0 | 0 | 0 |
| | Hypocreomycetidae | | | | |
| 0 | 5125 Hypocreales | 38 | 0 | 0 | 0 |
| F | 474943 Cordycipitaceae | 13 | 0 | 0 | 0 |
| G | 72232 lsaria | 9 | 0 | 0 | 0 |
| S | 114497 lsaria fumosorosea | 9 | 9 | 0 | 0 |
| G | 5581 Beauveria | 2 | 0 | 0 | 0 |
| S | 176275 Beauveria | 2 | 2 | 0 | 0 |
| | bassiana | | | | |
| G | 45234 Cordyceps | 1 | 0 | 0 | 0 |
| S | 73501 Cordyceps militaris | 1 | 1 | 0 | 0 |
| F | 34397 Clavicipitaceae | 11 | 0 | 0 | 0 |
| G | 243023 Pochonia | 6 | 0 | 0 | 0 |
| S | 280754 Pochonia | 6 | 6 | 0 | 0 |
| | chlamydosporia | | | | |
| G | 5529 Metarhizium | 5 | 0 | 0 | 0 |
| S | 1650735 Metarhizium | 2 | 2 | 0 | 0 |
| | majus | | | | |
| S | 92637 Metarhizium | 2 | 2 | 0 | 0 |
| | acridum | | | | |
| F | 5129 Hypocreaceae | 10 | 0 | 0 | 0 |
| G | 5543 Trichoderma | 10 | 0 | 0 | 0 |
| S | 51453 Trichoderma reesei | 7 | 7 | 0 | 0 |
| S | 63577 Trichoderma | 2 | 2 | 0 | 0 |
| | atroviride | | | | |
| S | 398673 Trichoderma | 1 | 1 | 0 | 0 |
| | gamsii | | | | |
| F | 474942 | 2 | 0 | 0 | 0 |
| | Ophiocordycipitaceae | | | | |
| G | 1052105 Purpureocillium | 2 | 0 | 0 | 0 |
| S | 33203 Purpureocillium | 2 | 2 | 0 | 0 |
| | lilacinum | | | | |
| F | 110618 Nectriaceae | 2 | 0 | 0 | 0 |
| G | 5506 Fusarium | 2 | 0 | 0 | 0 |
| G1 | 232080 Fusarium solani | 1 | 0 | 0 | 0 |
| | species complex | | | | |
| S | 140110 Nectria | 1 | 1 | 0 | 0 |
| | haematococca | | | | |
| G1 | 171631 Fusarium | 1 | 0 | 0 | 0 |
| | oxysporum species | | | | |
| | complex | | | | |
| S | 5507 Fusarium oxysporum | 1 | 1 | 0 | 0 |
| 0 | 1028384 Glomerellales | 47 | 0 | 0 | 0 |
| F | 681950 Glomerellaceae | 42 | 0 | 0 | 0 |
| G | 5455 Colletotrichum | 42 | 0 | 0 | 0 |
| S | 80884 Colletotrichum | 31 | 31 | 0 | 0 |
| | higginsianum | | | | |
| S | 31870 Colletotrichum | 7 | 7 | 0 | 0 |
| | graminicola | | | | |
| S | 474922 Colletotrichum | 3 | 3 | 0 | 0 |
| | gloeosporioides | | | | |
| S | 710243 Colletotrichum | 1 | 1 | 0 | 0 |
| | fioriniae | | | | |
| F | 1033978 | 5 | 0 | 0 | 0 |
| | Plectosphaerellaceae | | | | |
| G | 1036719 Verticillium | 5 | 0 | 0 | 0 |
| S | 27337 Verticillium dahliae | 4 | 4 | 0 | 0 |
| S | 1051613 Verticillium | 1 | 1 | 0 | 0 |
| | alfalfae | | | | |
| 0 | 5592 Microascales | 4 | 0 | 0 | 0 |
| F | 5593 Microascaceae | 4 | 0 | 0 | 0 |
| G | 41687 Scedosporium | 4 | 0 | 0 | 0 |
| S | 563466 Scedosporium | 4 | 4 | 0 | 0 |
| | apiospermum | | | | |
| C1 | 222544 Sordariomycetidae | 50 | 0 | 0 | 0 |
| 0 | 5139 Sordariales | 32 | 0 | 0 | 0 |
| F | 35718 Chaetomiaceae | 21 | 0 | 0 | 0 |
| G | 5149 Chaetomium | 10 | 0 | 0 | 0 |
| S | 38033 Chaetomium | 8 | 8 | 0 | 0 |
| | globosum | | | | |
| S | 209285 Chaetomium | 2 | 2 | 0 | 0 |
| | thermophilum | | | | |
| G | 35719 Thielavia | 6 | 0 | 0 | 0 |
| S | 35720 Thielavia terrestris | 6 | 6 | 0 | 0 |
| G | 1920207 | 4 | 0 | 0 | 0 |
| | Thermothelomyces | | | | |
| S | 78579 Thermothelomyces | 4 | 4 | 0 | 0 |
| | thermophila | | | | |
| F | 5148 Sordariaceae | 8 | 0 | 0 | 0 |
| G | 5140 Neurospora | 7 | 0 | 0 | 0 |
| S | 5141 Neurospora crassa | 6 | 6 | 0 | 0 |
| S | 40127 Neurospora | 1 | 1 | 0 | 0 |
| | tetrasperma | | | | |
| G | 5146 Sordaria | 1 | 0 | 0 | 0 |
| S | 5147 Sordaria macrospora | 1 | 1 | 0 | 0 |
| F | 42302 Lasiosphaeriaceae | 3 | 0 | 0 | 0 |
| G | 5144 Podospora | 3 | 0 | 0 | 0 |
| S | 5145 Podospora anserina | 3 | 3 | 0 | 0 |
| 0 | 5151 Ophiostomatales | 12 | 0 | 0 | 0 |
| F | 5152 Ophiostomataceae | 12 | 0 | 0 | 0 |
| G | 29907 Sporothrix | 11 | 0 | 0 | 0 |
| S | 29908 Sporothrix | 11 | 11 | 0 | 0 |
| | schenckii | | | | |
| G | 360145 Grosmannia | 1 | 0 | 0 | 0 |
| S | 226899 Grosmannia | 1 | 1 | 0 | 0 |
| | clavigera | | | | |
| 0 | 639021 Magnaporthales | 5 | 0 | 0 | 0 |
| F | 81093 Magnaporthaceae | 5 | 0 | 0 | 0 |
| G | 148303 Magnaporthe | 5 | 0 | 0 | 0 |
| S | 318829 Magnaporthe | 5 | 5 | 0 | 0 |
| | oryzae | | | | |
| 0 | 1775898 Togniniales | 1 | 0 | 0 | 0 |
| F | 1756146 Togniniaceae | 1 | 0 | 0 | 0 |
| G | 65412 Phaeoacremonium | 1 | 0 | 0 | 0 |
| S | 223192 Phaeoacremonium | 1 | 1 | 0 | 0 |
| | minimum | | | | |
| C1 | 222545 Xylariomycetidae | 6 | 0 | 0 | 0 |
| 0 | 37989 Xylariales | 6 | 0 | 0 | 0 |
| F | 42364 Diatrypaceae | 4 | 0 | 0 | 0 |
| G | 97095 Eutypa | 4 | 0 | 0 | 0 |
| S | 97096 Eutypa lata | 4 | 4 | 0 | 0 |
| F | 1812776 Sporocadaceae | 2 | 0 | 0 | 0 |
| G | 37840 Pestalotiopsis | 2 | 0 | 0 | 0 |
| S | 393283 Pestalotiopsis fici | 2 | 2 | 0 | 0 |
| C | 147548 Leotiomycetes | 28 | 0 | 0 | 0 |
| 0 | 5178 Helotiales | 22 | 0 | 0 | 0 |
| F | 76657 Dermateaceae | 9 | 0 | 0 | 0 |
| G | 324777 Marssonina | 9 | 0 | 0 | 0 |
| S | 698440 Marssonina | 9 | 9 | 0 | 0 |
| | brunnea | | | | |
| F | 28983 Sclerotiniaceae | 8 | 0 | 0 | 0 |
| G | 33196 Botrytis | 6 | 0 | 0 | 0 |
| S | 40559 Botrytis cinerea | 6 | 6 | 0 | 0 |
| G | 5179 Sclerotinia | 2 | 0 | 0 | 0 |
| S | 5180 Sclerotinia | 2 | 2 | 0 | 0 |
| | sclerotiorum | | | | |
| F | 5181 Helotiaceae | 4 | 0 | 0 | 0 |
| G | 101851 Glarea | 4 | 0 | 0 | 0 |
| S | 101852 Glarea Iozoyensis | 4 | 4 | 0 | 0 |
| 01 | 159639 Helotiales incertae | 1 | 0 | 0 | 0 |
| | sedis | | | | |
| G | 150173 Phialocephala | 1 | 0 | 0 | 0 |
| S | 149040 Phialocephala | 1 | 1 | 0 | 0 |
| | scopiformis | | | | |
| C1 | 221903 Leotiomycetes | 6 | 0 | 0 | 0 |
| | incertae sedis | | | | |
| F | 34379 Pseudeurotiaceae | 6 | 0 | 0 | 0 |
| G | 78156 Pseudogymnoascus | 6 | 0 | 0 | 0 |
| S | 342668 | 5 | 5 | 0 | 0 |
| | Pseudogymnoascus | | | | |
| | verrucosus | | | | |
| S | 655981 | 1 | 1 | 0 | 0 |
| | Pseudogymnoascus | | | | |
| | destructans | | | | |
| C | 147545 Eurotiomycetes | 174 | 0 | 0 | 0 |
| C1 | 451871 Eurotiomycetidae | 96 | 0 | 0 | 0 |
| 0 | 5042 Eurotiales | 65 | 0 | 0 | 0 |
| F | 1131492 Aspergillaceae | 59 | 0 | 0 | 0 |
| G | 5052 Aspergillus | 57 | 0 | 0 | 0 |
| S | 5061 Aspergillus niger | 44 | 44 | 0 | 0 |
| S | 5057 Aspergillus clavatus | 7 | 7 | 0 | 0 |
| S | 5062 Aspergillus oryzae | 2 | 2 | 0 | 0 |
| S | 5053 Aspergillus aculeatus | 2 | 2 | 0 | 0 |
| S | 36630 Aspergillus fischeri | 1 | 1 | 0 | 0 |
| S | 33178 Aspergillus terreus | 1 | 1 | 0 | 0 |
| G | 5073 Penicillium | 2 | 0 | 0 | 0 |
| G1 | 254878 Penicillium | 1 | 0 | 0 | 0 |
| | chrysogenum species | | | | |
| | complex | | | | |
| S | 1108849 Penicillium | 1 | 1 | 0 | 0 |
| | rubens | | | | |
| S | 36651 Penicillium | 1 | 1 | 0 | 0 |
| | digitatum | | | | |
| F | 28568 Trichocomaceae | 6 | 0 | 0 | 0 |
| G | 1132856 Rasamsonia | 3 | 0 | 0 | 0 |
| S | 68825 Rasamsonia | 3 | 3 | 0 | 0 |
| | emersonii | | | | |
| G | 5094 Talaromyces | 3 | 0 | 0 | 0 |
| S | 37727 Talaromyces | 2 | 2 | 0 | 0 |
| | mameffei | | | | |
| S | 28564 Talaromyces | 1 | 1 | 0 | 0 |
| | stipitatus | | | | |
| 0 | 33183 Onygenales | 31 | 0 | 0 | 0 |
| 01 | 1593277 Onygenales | 11 | 0 | 0 | 0 |
| | incertae sedis | | | | |
| G | 38946 Paracoccidioides | 7 | 0 | 0 | 0 |
| S | 1048829 Paracoccidioides | 5 | 5 | 0 | 0 |
| | Iutzii | | | | |
| S | 121759 Paracoccidioides | 2 | 2 | 0 | 0 |
| | brasiliensis | | | | |
| G | 5500 Coccidioides | 4 | 0 | 0 | 0 |
| S | 199306 Coccidioides | 3 | 3 | 0 | 0 |
| | posadasii | | | | |
| S | 5501 Coccidioides immitis | 1 | 1 | 0 | 0 |
| F | 34384 Arthrodermataceae | 11 | 0 | 0 | 0 |
| G | 5550 Trichophyton | 5 | 0 | 0 | 0 |
| S | 63400 Trichophyton | 3 | 3 | 0 | 0 |
| | benhamiae | | | | |
| S | 5551 Trichophyton | 1 | 1 | 0 | 0 |
| | rubrum | | | | |
| G | 34392 Microsporum | 4 | 0 | 0 | 0 |
| S | 63405 Microsporum canis | 4 | 4 | 0 | 0 |
| G | 1915381 Nannizzia | 2 | 0 | 0 | 0 |
| S | 63402 Nannizzia gypsea | 2 | 2 | 0 | 0 |
| F | 299071 Ajellomycetaceae | 9 | 0 | 0 | 0 |
| G | 5036 Histoplasma | 5 | 0 | 0 | 0 |
| S | 5037 Histoplasma | 5 | 5 | 0 | 0 |
| | capsulatum | | | | |
| G | 229219 Blastomyces | 4 | 0 | 0 | 0 |
| S | 1681229 Blastomyces | 4 | 4 | 0 | 0 |
| | gilchristii | | | | |
| C1 | 451870 | 78 | 0 | 0 | 0 |
| | Chaetothyriomycetidae | | | | |
| 0 | 34395 Chaetothyriales | 76 | 0 | 0 | 0 |
| F | 43219 Herpotrichiellaceae | 74 | 0 | 0 | 0 |
| G | 5583 Exophiala | 62 | 0 | 0 | 0 |
| S | 348802 Exophiala | 53 | 53 | 0 | 0 |
| | xenobiotica | | | | |
| S | 215243 Exophiala | 5 | 5 | 0 | 0 |
| | oligosperma | | | | |
| S | 91928 Exophiala spinifera | 3 | 3 | 0 | 0 |
| S | 5970 Exophiala | 1 | 1 | 0 | 0 |
| | dermatitidis | | | | |
| G | 40354 Fonsecaea | 6 | 0 | 0 | 0 |
| S | 40355 Fonsecaea pedrosoi | 5 | 5 | 0 | 0 |
| G | 82105 Cladophialophora | 3 | 0 | 0 | 0 |
| S | 569365 Cladophialophora | 2 | 2 | 0 | 0 |
| | immunda | | | | |
| S | 86049 Cladophialophora | 1 | 1 | 0 | 0 |
| | carrionii | | | | |
| G | 43220 Capronia | 2 | 0 | 0 | 0 |
| S | 43229 Capronia coronata | 2 | 2 | 0 | 0 |
| G | 5600 Phialophora | 1 | 0 | 0 | 0 |
| S | 1664694 Phialophora attae | 1 | 1 | 0 | 0 |
| F | 1341112 | 2 | 0 | 0 | 0 |
| | Cyphellophoraceae | | | | |
| G | 226991 Cyphellophora | 2 | 0 | 0 | 0 |
| S | 293227 Cyphellophora | 2 | 2 | 0 | 0 |
| | europaea | | | | |
| 0 | 146291 Verrucariales | 2 | 0 | 0 | 0 |
| F | 146292 Verrucariaceae | 2 | 0 | 0 | 0 |
| G | 364710 Endocarpon | 2 | 0 | 0 | 0 |
| S | 364733 Endocarpon | 2 | 2 | 0 | 0 |
| | pusillum | | | | |
| P4 | 715962 dothideomyceta | 48 | 0 | 0 | 0 |
| C | 147541 Dothideomycetes | 48 | 0 | 0 | 0 |
| C1 | 451868 | 29 | 0 | 0 | 0 |
| | Pleosporomycetidae | | | | |
| 0 | 92860 Pleosporales | 29 | 0 | 0 | 0 |
| 01 | 715340 Pleosporineae | 25 | 0 | 0 | 0 |
| F | 28556 Pleosporaceae | 21 | 0 | 0 | 0 |
| G | 39952 Setosphaeria | 8 | 0 | 0 | 0 |
| S | 93612 Setosphaeria turcica | 8 | 8 | 0 | 0 |
| G | 5027 Pyrenophora | 7 | 0 | 0 | 0 |
| S | 45151 Pyrenophora tritici- | 4 | 4 | 0 | 0 |
| | repentis | | | | |
| S | 53485 Pyrenophora teres | 1 | 1 | 0 | 0 |
| G | 33194 Bipolaris | 4 | 0 | 0 | 0 |
| S | 40125 Bipolaris victoriae | 2 | 2 | 0 | 0 |
| S | 101162 Bipolaris oryzae | 1 | 1 | 0 | 0 |
| S | 45130 Bipolaris | 1 | 1 | 0 | 0 |
| | sorokiniana | | | | |
| G | 5598 Alternaria | 1 | 0 | 0 | 0 |
| G1 | 187734 Alternaria | 1 | 0 | 0 | 0 |
| | alternata group | | | | |
| S | 5599 Alternaria alternata | 1 | 1 | 0 | 0 |
| F | 34374 Leptosphaeriaceae | 3 | 0 | 0 | 0 |
| G | 5021 Leptosphaeria | 3 | 0 | 0 | 0 |
| G1 | 220671 Leptosphaeria | 3 | 0 | 0 | 0 |
| | maculans species complex | | | | |
| S | 5022 Leptosphaeria | 3 | 3 | 0 | 0 |
| | maculans | | | | |
| F | 5020 Phaeosphaeriaceae | 1 | 0 | 0 | 0 |
| G | 1351751 Parastagonospora | 1 | 0 | 0 | 0 |
| S | 13684 Parastagonospora | 1 | 1 | 0 | 0 |
| | nodorum | | | | |
| 01 | 1255046 Massarineae | 4 | 0 | 0 | 0 |
| F | 221678 | 4 | 0 | 0 | 0 |
| | Didymosphaeriaceae | | | | |
| G | 125369 Paraphaeosphaeria | 4 | 0 | 0 | 0 |
| S | 1460663 | 4 | 4 | 0 | 0 |
| | Paraphaeosphaeria | | | | |
| | sporulosa | | | | |
| C1 | 451867 | 12 | 0 | 0 | 0 |
| | Dothideomycetidae | | | | |
| 0 | 134362 Capnodiales | 10 | 0 | 0 | 0 |
| F | 93133 | 10 | 0 | 0 | 0 |
| | Mycosphaerellaceae | | | | |
| G | 131324 Pseudocercospora | 6 | 0 | 0 | 0 |
| S | 1873960 | 6 | 6 | 0 | 0 |
| | Pseudocercospora fijiensis | | | | |
| G | 237179 Sphaerulina | 4 | 0 | 0 | 0 |
| S | 85929 Sphaerulina musiva | 4 | 4 | 0 | 0 |
| 0 | 5014 Dothideales | 2 | 0 | 0 | 0 |
| F | 1570301 Saccotheciaceae | 2 | 0 | 0 | 0 |
| G | 5579 Aureobasidium | 2 | 0 | 0 | 0 |
| S | 1042127 Aureobasidium | 1 | 1 | 0 | 0 |
| | subglaciale | | | | |
| S | 559561 Aureobasidium | 1 | 1 | 0 | 0 |
| | namibiae | | | | |
| C1 | 159987 Dothideomycetes | 7 | 0 | 0 | 0 |
| | incertae sedis | | | | |
| 0 | 451869 Botryosphaeriales | 4 | 0 | 0 | 0 |
| F | 45131 Botryosphaeriaceae | 4 | 0 | 0 | 0 |
| G | 407951 Neofusicoccum | 2 | 0 | 0 | 0 |
| S | 310453 Neofusicoccum | 2 | 2 | 0 | 0 |
| | parvum | | | | |
| G | 66735 Diplodia | 2 | 0 | 0 | 0 |
| S | 236234 Diplodia corticola | 2 | 2 | 0 | 0 |
| G | 1955842 Coniosporium | 2 | 0 | 0 | 0 |
| S | 61459 Coniosporium | 2 | 2 | 0 | 0 |
| | apollinis | | | | |
| 0 | 1111111 Venturiales | 1 | 0 | 0 | 0 |
| F | 1111112 | 1 | 0 | 0 | 0 |
| | Sympoventuriaceae | | | | |
| G | 1494215 Verruconis | 1 | 0 | 0 | 0 |
| S | 253628 Verruconis | 1 | 1 | 0 | 0 |
| | gallopava | | | | |
| C | 1217819 Xylonomycetes | 1 | 0 | 0 | 0 |
| 0 | 1217820 Xylonomycetales | 1 | 0 | 0 | 0 |
| F | 1217822 | 1 | 0 | 0 | 0 |
| | Xylonomycetaceae | | | | |
| G | 1217823 Xylona | 1 | 0 | 0 | 0 |
| S | 1217826 Xylona heveae | 1 | 1 | 0 | 0 |
| C | 189478 Orbiliomycetes | 9 | 0 | 0 | 0 |
| 0 | 189479 Orbiliales | 9 | 0 | 0 | 0 |
| F | 47021 Orbiliaceae | 9 | 0 | 0 | 0 |
| G | 314122 Dactylellina | 5 | 0 | 0 | 0 |
| S | 430498 Dactylellina | 5 | 5 | 0 | 0 |
| | haptotyla | | | | |
| G | 13348 Arthrobotrys | 4 | 0 | 0 | 0 |
| S | 13349 Arthrobotrys | 4 | 4 | 0 | 0 |
| | oligospora | | | | |
| C | 147549 Pezizomycetes | 1 | 0 | 0 | 0 |
| 0 | 5185 Pezizales | 1 | 0 | 0 | 0 |
| F | 40289 Tuberaceae | 1 | 0 | 0 | 0 |
| G | 36048 Tuber | 1 | 0 | 0 | 0 |
| S | 39416 Tuber | 1 | 1 | 0 | 0 |
| | melanosporum | | | | |
| P2 | 147537 Saccharomycotina | 138 | 0 | 0 | 0 |
| C | 4891 Saccharomycetes | 138 | 0 | 0 | 0 |
| 0 | 4892 Saccharomycetales | 138 | 0 | 0 | 0 |
| F | 4893 Saccharomycetaceae | 94 | 0 | 0 | 0 |
| G | 4930 Saccharomyces | 86 | 0 | 0 | 0 |
| S | 4932 Saccharomyces | 84 | • 84 | 0 | 0 |
| | cerevisiae | | | | |
| S | 1080349 Saccharomyces | 2 | 2 | 0 | 0 |
| | eubayanus | | | | |
| G | 374468 Nakaseomyces | 3 | 0 | 0 | 0 |
| G1 | 600669 | 3 | 0 | 0 | 0 |
| | Nakaseomyces/Candida | | | | |
| | Glade | | | | |
| S | 5478 [Candida] glabrata | 3 | 3 | 0 | 0 |
| G | 113604 Tetrapisispora | 3 | 0 | 0 | 0 |
| S | 1071379 Tetrapisispora | 3 | 3 | 0 | 0 |
| | blattae | | | | |
| G | 278028 Naumovozyma | 1 | 0 | 0 | 0 |
| S | 27289 Naumovozyma | 1 | 1 | 0 | 0 |
| | dairenensis | | | | |
| G | 33170 Eremothecium | 1 | 0 | 0 | 0 |
| S | 33169 Eremothecium | 1 | 1 | 0 | 0 |
| | gossypii | | | | |
| F | 44277 Ascoideaceae | 18 | 0 | 0 | 0 |
| G | 27308 Ascoidea | 18 | 0 | 0 | 0 |
| S | 54195 Ascoidea rubescens | 18 | 18 | 0 | 0 |
| F | 766764 | 11 | 0 | 0 | 0 |
| | Debaryomycetaceae | | | | |
| F1 | 1535325 | 8 | 0 | 0 | 0 |
| | Candida/Lodderomyces | | | | |
| | Glade | | | | |
| G | 36913 Lodderomyces | 5 | 0 | 0 | 0 |
| S | 36914 Lodderomyces | 5 | 5 | 0 | 0 |
| | elongisporus | | | | |
| G | 1535326 Candida | 3 | 0 | 0 | 0 |
| S | 42374 Candida | 2 | 2 | 0 | 0 |
| | dubliniensis | | | | |
| S | 5482 Candida tropicalis | 1 | 1 | 0 | 0 |
| G | 412764 Spathaspora | 2 | 0 | 0 | 0 |
| S | 340170 Spathaspora | 2 | 2 | 0 | 0 |
| | passalidarum | | | | |
| G | 766748 Babjeviella | 1 | 0 | 0 | 0 |
| S | 45609 Babjeviella | 1 | 1 | 0 | 0 |
| | inositovora | | | | |
| F | 115784 Phaffomycetaceae | 10 | 0 | 0 | 0 |
| G | 599737 | 8 | 0 | 0 | 0 |
| | Wickerhamomyces | | | | |
| S | 1041607 | 8 | 8 | 0 | 0 |
| | Wickerhamomyces ciferrii | | | | |
| G | 460517 Komagataella | 2 | 0 | 0 | 0 |
| S | 460519 Komagataella | 2 | 2 | 0 | 0 |
| | phaffii | | | | |
| F | 34353 Dipodascaceae | 2 | 0 | 0 | 0 |
| G | 4951 Yarrowia | 2 | 0 | 0 | 0 |
| S | 4952 Yarrowia lipolytica | 2 | 2 | 0 | 0 |
| F | 1156497 Pichiaceae | 1 | 0 | 0 | 0 |
| G | 4919 Pichia | 1 | 0 | 0′ | 0 |
| S | 4909 Pichia kudriavzevii | 1 | 1 | 0 | 0 |
| F | 27319 Metschnikowiaceae | 1 | 0 | 0 | 0 |
| G | 36910 Clavispora | 1 | 0 | 0 | 0 |
| S | 36911 Clavispora | 1 | 1 | 0 | 0 |
| | Iusitaniae | | | | |
| P1 | 451866 Taphrinomycotina | 2 | 0 | 0 | 0 |
| C | 147554 | 2 | 0 | 0 | 0 |
| | Schizosaccharomycetes | | | | |
| 0 | 34346 | 2 | 0 | 0 | 0 |
| | Schizosaccharomycetales | | | | |
| F | 4894 | 2 | 0 | 0 | 0 |
| | Schizosaccharomycetaceae | | | | |
| G | 4895 | 2 | 0 | 0 | 0 |
| | Schizosaccharomyces | | | | |
| S | 866546 | 1 | 1 | 0 | 0 |
| | Schizosaccharomyces | | | | |
| | cryophilus | | | | |
| S | 4899 | 1 | 1 | 0 | 0 |
| | Schizosaccharomyces | | | | |
| | octosporus | | | | |
| P | 5204 Basidiomycota | 74 | 0 | 0 | 0 |
| P1 | 5302 Agaricomycotina | 47 | 0 | 0 | 0 |
| C | 155619 Agaricomycetes | 35 | 0 | 0 | 0 |
| C1 | 355688 Agaricomycetes | 23 | 0 | 0 | 0 |
| | incertae sedis | | | | |
| 0 | 5303 Polyporales | 15 | 0 | 0 | 0 |
| F | 396331 Phanerochaetaceae | 6 | 0 | 0 | 0 |
| G | 5305 Phanerochaete | 6 | 0 | 0 | 0 |
| S | 231932 Phanerochaete | 6 | 6 | 0 | 0 |
| | camosa | | | | |
| F | 2028214 Dacryobolaceae | 5 | 0 | 0 | 0 |
| G | 83235 Postia | 5 | 0 | 0 | 0 |
| S | 104341 Postia placenta | 5 | 5 | 0 | 0 |
| F | 5317 Polyporaceae | 4 | 0 | 0 | 0 |
| G | 5324 Trametes | 4 | 0 | 0 | 0 |
| S | 5325 Trametes versicolor | 4 | 4 | 0 | 0 |
| 0 | 5226 Auriculariales | 6 | 0 | 0 | 0 |
| F | 5227 Auriculariaceae | 6 | 0 | 0 | 0 |
| G | 5230 Auricularia | 6 | 0 | 0 | 0 |
| S | 1331295 Auricularia | 6 | 6 | 0 | 0 |
| | subglabra | | | | |
| 0 | 452342 Russulales | 1 | 0 | 0 | 0 |
| F | 40420 Bondarzewiaceae | 1 | 0 | 0 | 0 |
| G | 13562 Heterobasidion | 1 | 0 | 0 | 0 |
| G1 | 256003 Heterobasidion | 1 | 0 | 0 | 0 |
| | annosum species complex | | | | |
| S | 984962 Heterobasidion | 1 | 1 | 0 | 0 |
| | irregulare | | | | |
| 0 | 452339 Gloeophyllales | 1 | 0 | 0 | 0 |
| F | 452340 Gloeophyllaceae | 1 | 0 | 0 | 0 |
| G\ | 40443 Gloeophyllum | 1 | 0 | 0 | 0 |
| S | 104355 Gloeophyllum | 1 | 1 | 0 | 0 |
| | trabeum | | | | |
| C1 | 452333 Agaricomycetidae | 12 | 0 | 0 | 0 |
| 0 | 68889 Boletales | 8 | 0 | 0 | 0 |
| 01 | 227334 Coniophorineae | 8 | 0 | 0 | 0 |
| F | 389951 Serpulaceae | 6 | 0 | 0 | 0 |
| G | 80744 Serpula | 6 | 0 | 0 | 0 |
| S | 85982 Serpula lacrymans | 6 | 6 | 0 | 0 |
| F | 80634 Coniophoraceae | 2 | 0 | 0 | 0 |
| G | 80635 Coniophora | 2 | 0 | 0 | 0 |
| S | 80637 Coniophora puteana | 2 | 2 | 0 | 0 |
| 0 | 5338 Agaricales | 4 | 0 | 0 | 0 |
| F | 184208 Psathyrellaceae | 2 | 0 | 0 | 0 |
| G | 184431 Coprinopsis | 2 | 0 | 0 | 0 |
| S | 5346 Coprinopsis cinerea | 2 | 2 | 0 | 0 |
| F | 5339 Agaricaceae | 2 | 0 | 0 | 0 |
| G | 5340 Agaricus | 2 | 0 | 0 | 0 |
| S | 5341 Agaricus bisporus | 2 | 2 | 0 | 0 |
| C | 155616 Tremellomycetes | 10 | 0 | 0 | 0 |
| 0 | 1851469 Trichosporonales | 6 | 0 | 0 | 0 |
| F | 1759442 | 6 | 0 | 0 | 0 |
| | Trichosporonaceae | | | | |
| G | 1838142 | 4 | 0 | 0 | 0 |
| | Cutaneotrichosporon | | | | |
| S | 879819 | 4 | 4 | 0 | 0 |
| | Cutaneotrichosporon | | | | |
| | oleaginosus | | | | |
| G | 5552 Trichosporon | 2 | 0 | 0 | 0 |
| S | 82508 Trichosporon asahii | 2 | 2 | 0 | 0 |
| 0 | - 5234 Tremellales | 4 | 0 | 0 | 0 |
| F | 1884633 Cryptococcaceae | 4 | 0 | 0 | 0 |
| G | 490731 Kwoniella | 2 | 0 | 0 | 0 |
| S | 463800 Kwoniella | 1 | 1 | 0 | 0 |
| | mangrovensis | | | | |
| S | 324770 Kwoniella | 1 | 1 | 0 | 0 |
| | dejecticola | | | | |
| G | 5206 Cryptococcus | 2 | 0 | 0 | 0 |
| G1 | 1884637 Cryptococcus | 2 | 0 | 0 | 0 |
| | gattii species complex | | | | |
| S | 37769 Cryptococcus gattii | 2 | 2 | 0 | 0 |
| | VGI | | | | |
| C | 431957 Wallemiomycetes | 1 | 0 | 0 | 0 |
| 0 | 431958 Wallemiales | 1 | 0 | 0 | 0 |
| 01 | 431959 Wallemiales | 1 | 0 | 0 | 0 |
| | incertae sedis | | | | |
| G | 148959 Wallemia | 1 | 0 | 0 | 0 |
| S | 245174 Wallemia | 1 | 1 | 0 | 0 |
| | ichthyophaga | | | | |
| P1 | 452284 | 16 | 0 | 0 | 0 |
| | Ustilaginomycotina | | | | |
| C | 5257 Ustilaginomycetes | 10 | 0 | 0 | 0 |
| 0 | 5267 Ustilaginales | 10 | 0 | 0 | 0 |
| F | 5268 Ustilaginaceae | 10 | 0 | 0 | 0 |
| G | 1392992 Anthracocystis | 5 | 0 | 0 | 0 |
| S | 84751 Anthracocystis | 5 | 5 | 0 | 0 |
| | flocculosa | | | | |
| G | 63298 Pseudozyma | 2 | 0 | 0 | 0 |
| S | 327079 Pseudozyma | 2 | 2 | 0 | 0 |
| | hubeiensis | | | | |
| G | 1804794 Kalmanozyma | 1 | 0 | 0 | 0 |
| S | 1392244 Kalmanozyma | 1 | 1 | 0 | 0 |
| | brasiliensis | | | | |
| G | 63261 Moesziomyces | 1 | 0 | 0 | 0 |
| S | 84753 Moesziomyces | 1 | 1 | 0 | 0 |
| | antarcticus | | | | |
| C | 1538075 | 5 | 0 | 0 | 0 |
| | Malasseziomycetes | | | | |
| 0 | 162474 Malasseziales | 5 | 0 | 0 | 0 |
| F | 742845 Malasseziaceae | 5 | 0 | 0 | 0 |
| G | 55193 Malassezia | 5 | 0 | 0 | 0 |
| S | 76773 Malassezia globosa | 3 | 3 | 0 | 0 |
| S | 76777 Malassezia | 2 | 2 | 0 | 0 |
| | sympodialis | | | | |
| C | 452283 | 1 | 0 | 0 | 0 |
| | Exobasidiomycetes | | | | |
| 0 | 62913 Georgefischeriales | 1 | 0 | 0 | 0 |
| F | 62919 Tilletiariaceae | 1 | 0 | 0 | 0 |
| G | 5280 Tilletiaria | 1 | 0 | 0 | 0 |
| S | 5281 Tilletiaria anomala | 1 | 1 | 0 | 0 |
| P1 | 29000 Pucciniomycotina | 8 | 0 | 0 | 0 |
| C | 162484 Pucciniomycetes | 5 | 0 | 0 | 0 |
| 0 | 5258 Pucciniales | 5 | 0 | 0 | 0 |
| F | 5262 Pucciniaceae | 5 | 0 | 0 | 0 |
| G | 5296 Puccinia | 5 | 0 | 0 | 0 |
| S | 5297 Puccinia graminis | 5 | 5 | 0 | 0 |
| C | 162481 | 2 | 0 | 0 | 0 |
| | Microbotryomycetes | | | | |
| 0 | 231213 Sporidiobolales | 2 | 0 | 0 | 0 |
| F | 1799696 Sporidiobolaceae | 2 | 0 | 0 | 0 |
| G | 5533 Rhodotorula | 2 | 0 | 0 | 0 |
| S | 29898 Rhodotorula | 2 | 2 | 0 | 0 |
| | graminis | | | | |
| C | 432025 Mixiomycetes | 1 | 0 | 0 | 0 |
| 0 | 432026 Mixiales | 1 | 0 | 0 | 0 |
| F | 165795 Mixiaceae | 1 | 0 | 0 | 0 |
| G | 34348 Mixia | 1 | 0 | 0 | 0 |
| S | 34349 Mixia osmundae | 1 | 1 | 0 | 0 |
| P | 6029 Microsporidia | 74 | 0 | 0 | 0 |
| P1 | 6032 Apansporoblastina | 74 | 0 | 0 | 0 |
| F | 27970 Enterocytozoonidae | 74 | 0 | 0 | 0 |
| G | 27971 Enterocytozoon | 74 | 0 | 0 | 0 |
| S | 31281 Enterocytozoon | 74 | 74 | 0 | 0 |
| | bieneusi | | | | |
| P | 1913637 Mucoromycota | 10 | 0 | 0 | 0 |
| P1 | 1137986 | 9 | 0 | 0 | 0 |
| | Mortierellomycotina | | | | |
| 0 | 214503 Mortierellales | 9 | 0 | 0 | 0 |
| F | 4854 Mortierellaceae | 9 | 0 | 0 | 0 |
| G | 299330 Lobosporangium | 9 | 0 | 0 | 0 |
| S | 64571 Lobosporangium | 9 | 9 | 0 | 0 |
| | transversale | | | | |
| P1 | 451507 Mucoromycotina | 1 | 0 | 0 | 0 |
| 0 | 4827 Mucorales | 1 | 0 | 0 | 0 |
| F | 1344966 | 1 | 0 | 0 | 0 |
| | Phycomycetaceae | | | | |
| G | 4836 Phycomyces | 1 | 0 | 0 | 0 |
| S | 4837 Phycomyces | 1 | 1 | 0 | 0 |
| | blakesleeanus | | | | |
| P | 4761 Chytridiomycota | 1 | 0 | 0 | 0 |
| C | 451435 Chytridiomycetes | 1 | 0 | 0 | 0 |
| 0 | 34478 Spizellomycetales | 1 | 0 | 0 | 0 |
| F | 34479 Spizellomycetaceae | 1 | 0 | 0 | 0 |
| G | 4815 Spizellomyces | 1 | 0 | 0 | 0 |
| S | 109760 Spizellomyces | 1 | 1 | 0 | 0 |
| | punctatus | | | | |
| D1 | 33630 Alveolata | 460 | 0 | 0 | 0 |
| P | 5794 Apicomplexa | 411 | 0 | 0 | 0 |
| C | 422676 Aconoidasida | 264 | 0 | 0 | 0 |
| 0 | 5819 Haemosporida | 262 | 0 | 0 | 0 |
| F | 1639119 Plasmodiidae | 262 | 0 | 0 | 0 |
| G | 5820 Plasmodium | 262 | 0 | 0 | 0 |
| G1 | 418101 Plasmodium | 166 | 0 | 0 | 0 |
| | (Vinckeia) | | | | |
| S | 5821 Plasmodium berghei | 110 | 110 | 0 | 0 |
| S | 5861 Plasmodium yoelii | 51 | 51 | 0 | 0 |
| S | 5825 Plasmodium | 5 | 5 | 0 | 0 |
| | chabaudi | | | | |
| G1 | 418107 Plasmodium | 58 | 0 | 0 | 0 |
| | (Laverania) | | | | |
| S | 5833 Plasmodium | 24 | 24 | 0 | 0 |
| | falciparum | | | | |
| S | 5854 Plasmodium | 18 | 18 | 0 | 0 |
| | reichenowi | | | | |
| S | 647221 Plasmodium | 15 | 15 | 0 | 0 |
| | gaboni | | | | |
| G1 | 418103 Plasmodium | 20 | 0 | 0 | 0 |
| | (Plasmodium) | | | | |
| S | 5827 Plasmodium | 6 | 6 | 0 | 0 |
| | cynomolgi | | | | |
| S | 5857 Plasmodium fragile | 4 | 4 | 0 | 0 |
| S | 5850 Plasmodium | 4 | 4 | 0 | 0 |
| | knowlesi | | | | |
| S | 5855 Plasmodium vivax | 3 | 3 | 0 | 0 |
| S | 52288 Plasmodium inui | , 2 | 2 | 0 | 0 |
| S | 208452 Plasmodium | 17 | 17 | 0 | 0 |
| | coatneyi | | | | |
| C1 | 5863 Piroplasmida | 2 | 0 | 0 | 0 |
| F | 32594 Babesiidae | 2 | 0 | 0 | 0 |
| G | 5864 Babesia | 2 | 0 | 0 | 0 |
| S | 5866 Babesia bigemina | 2 | 2 | 0 | 0 |
| P1 | 1280412 Conoidasida | 146 | 0 | 0 | 0 |
| C | 5796 Coccidia | 139 | 0 | 0 | 0 |
| 0 | 75739 Eucoccidiorida | 139 | 0 | 0 | 0 |
| 01 | 423054 Eimeriorina | 139 | 0 | 0 | 0 |
| F | 5799 Eimeriidae | 111 | 0 | 0 | 0 |
| G | 5800 Eimeria | 111 | 0 | 0 | 0 |
| S | 44415 Eimeria mitis | 50 | 50 | 0 | 0 |
| S | 5804 Eimeria maxima | 31 | 31 | 0 | 0 |
| S | 5801 Eimeria acervulina | 11 | 11 | 0 | 0 |
| S | 51315 Eimeria necatrix | 9 | 9 | 0 | 0 |
| S | 5802 Eimeria tenella | 9 | 9 | 0 | 0 |
| F | 35082 Cryptosporidiidae | 17 | 0 | 0 | 0 |
| G | 5806 Cryptosporidium | 17 | 0 | 0 | 0 |
| S | 5808 Cryptosporidium | 1 | 1 | 0 | 0 |
| | muris | | | | |
| F | 5809 Sarcocystidae | 11 | 0 | 0 | 0 |
| G | 99157 Hammondia | 4 | 0 | 0 | 0 |
| S | 99158 Hammondia | 4 | 4 | 0 | 0 |
| | hammondi | | | | |
| G | 29175 Neospora | 4 | 0 | 0 | 0 |
| S | 29176 Neospora caninum | 4 | 4 | 0 | 0 |
| G | 5810 Toxoplasma | 3 | 0 | 0 | 0 |
| S | 5811 Toxoplasma gondii | 3 | 3 | 0 | 0 |
| C | 35086 Gregarinasina | 7 | 0 | 0 | 0 |
| 0 | 35087 Eugregarinorida | 7 | 0 | 0 | 0 |
| F | 35088 Gregarinidae | 7 | 0 | 0 | 0 |
| G | 35089 Gregarina | 7 | 0 | 0 | 0 |
| S | 110365 Gregarina | 7 | 7 | 0 | 0 |
| | niphandrodes | | | | |
| D2 | 5878 Ciliophora | 47 | 0 | 0 | 0 |
| D3 | 431838 | 47 | 0 | 0 | 0 |
| | Intramacronucleata | | | | |
| C | 6020 Oligohymenophorea | 47 | 0 | 0 | 0 |
| 0 | 31277 Hymenostomatida | 43 | 0 | 0 | 0 |
| 01 | 37090 Ophryoglenina | 33 | 0 | 0 | 0 |
| G | 5931 lchthyophthirius | 33 | 0 | 0 | 0 |
| S | 5932 lchthyophthirius | 33 | 33 | 0 | 0 |
| | multifiliis | | | | |
| 01 | 37093 Tetrahymenina | 10 | 0 | 0 | 0 |
| F | 291294 Tetrahymenidae | 10 | 0 | 0 | 0 |
| G | 5890 Tetrahymena | 10 | 0 | 0 | 0 |
| S | 5911 Tetrahymena | 10 | 10 | 0 | 0 |
| | thermophila | | | | |
| 0 | 33825 Peniculida | 4 | 0 | 0 | 0 |
| F | 340080 Parameciidae | 4 | 0 | 0 | 0 |
| G | 5884 Paramecium | 4 | 0 | 0 | 0 |
| S | 5888 Paramecium | 4 | 4 | 0 | 0 |
| | tetraurelia | | | | |
| D2 | 27997 Perkinsea | 2 | 0 | 0 | 0 |
| 0 | 27998 Perkinsida | 2 | 0 | 0 | 0 |
| F | 27999 Perkinsidae | 2 | 0 | 0 | 0 |
| G | 28000 Perkinsus | 2 | 0 | 0 | 0 |
| S | 31276 Perkinsus marinus | 2 | 2 | 0 | 0 |
| D1 | 554915 Amoebozoa | 351 | 0 | 0 | 0 |
| D2 | 142796 Mycetozoa | 196 | 0 | 0 | 0 |
| 0 | 33083 Dictyosteliida | 196 | 0 | 0 | 0 |
| G | 5782 Dictyostelium | 146 | 0 | 0 | 0 |
| S | 44689 Dictyostelium | 84 | 84 | 0 | 0 |
| | discoideum | | | | |
| S | 5786 Dictyostelium | 57 | 57 | 0 | 0 |
| | purpureum | | | | |
| S | 261658 Dictyostelium | 5 | 5 | 0 | 0 |
| | fasciculatum | | | | |
| G | 13641 Polysphondylium | 46 | 0 | 0 | 0 |
| S | 13642 Polysphondylium | 46 | 46 | 0 | 0 |
| | pallidum | | | | |
| G | 133407 Acytostelium | 4 | 0 | 0 | 0 |
| S | 361139 Acytostelium | 4 | 4 | 0 | 0 |
| | subglobosum | | | | |
| D2 | 555406 Archamoebae | 145 | 0 | 0 | 0 |
| F | 33084 Entamoebidae | 145 | 0 | 0 | 0 |
| G | 5758 Entamoeba | 145 | 0 | 0 | 0 |
| S | 46681 Entamoeba dispar | 112 | 112 | 0 | 0 |
| S | 33085 Entamoeba | 24 | 24 | 0 | 0 |
| | invadens | | | | |
| S | 5759 Entamoeba | 9 | 9 | 0 | 0 |
| | histolytica | | | | |
| D2 | 555280 Discosea | 10 | 0 | 0 | 0 |
| 0 | 1485168 Longamoebia | 10 | 0 | 0 | 0 |
| 01 | 555407 Centramoebida | 10 | 0 | 0 | 0 |
| F | 33677 Acanthamoebidae | 10 | 0 | 0 | 0 |
| G | 5754 Acanthamoeba | 10 | 0 | 0 | 0 |
| S | 5755 Acanthamoeba | 10 | 10 | 0 | 0 |
| | castellanii | | | | |
| D1 | 33634 Stramenopiles | 57 | 0 | 0 | 0 |
| C | 4762 Oomycetes | 31 | 0 | 0 | 0 |
| 0 | 4763 Saprolegniales | 16 | 0 | 0 | 0 |
| F | 4764 Saprolegniaceae | 16 | 0 | 0 | 0 |
| G | 4769 Saprolegnia | 12 | 0 | 0 | 0 |
| S | 112098 Saprolegnia | 12 | 12 | 0 | 0 |
| | diclina | | | | |
| G | 100860 Aphanomyces | 4 | 0 | 0 | 0 |
| S | 157072 Aphanomyces | 3 | 3 | 0 | 0 |
| | invadans | | | | |
| S | 112090 Aphanomyces | 1 | 1 | 0 | 0 |
| | astaci | | | | |
| 0 | 4776 Peronosporales | 15 | 0 | 0 | 0 |
| G | 4783 Phytophthora | 15 | 0 | 0 | 0 |
| S | 67593 Phytophthora sojae | 7 | 7 | 0 | 0 |
| S | 4787 Phytophthora | 7 | 7 | 0 | 0 |
| | infestans | | | | |
| C | 35675 Pelagophyceae | 12 | 0 | 0 | 0 |
| 0 | 54409 Pelagomonadales | 12 | 0 | 0 | 0 |
| G | 44055 Aureococcus | 12 | 0 | 0 | 0 |
| S | 44056 Aureococcus | 12 | 12 | 0 | 0 |
| | anophagefferens | | | | |
| G | 12967 Blastocystis | 7 | 0 | 0 | 0 |
| S | 12968 Blastocystis | 7 | 7 | 0 | 0 |
| | hominis | | | | |
| P | 2836 Bacillariophyta | 5 | 0 | 0 | 0 |
| C | 33836 | 5 | 0 | 0 | 0 |
| | Coscinodiscophyceae | | | | |
| C1 | 33846 | 5 | 0 | 0 | 0 |
| | Thalassiosirophycidae | | | | |
| 0 | 33847 Thalassiosirales | 5 | 0 | 0 | 0 |
| F | 29202 Thalassiosiraceae | 5 | 0 | 0 | 0 |
| G | 35127 Thalassiosira | 5 | 0 | 0 | 0 |
| S | 35128 Thalassiosira | 5 | 5 | 0 | 0 |
| | pseudonana | | | | |
| P | 5747 Eustigmatophyceae | 2 | 0 | 0 | 0 |
| 0 | 425074 Eustigmatales | 2 | 0 | 0 | 0 |
| F | 425072 Monodopsidaceae | 2 | 0 | 0 | 0 |
| G | 5748 Nannochloropsis | 2 | 0 | 0 | 0 |
| S | 72520 Nannochloropsis | 2 | 2 | 0 | 0 |
| | gaditana | | | | |
| D1 | 33682 Euglenozoa | 66 | 0 | 0 | 0 |
| 0 | 5653 Kinetoplastida | 66 | 0 | 0 | 0 |
| F | 5654 Trypanosomatidae | 66 | 0 | 0 | 0 |
| Fl | 1286322 Leishmaniinae | 27 | 0 | 0 | 0 |
| G | 5658 Leishmania | 25 | 0 | 0 | 0 |
| G1 | 38568 Leishmania | 20 | 0 | 0 | 0 |
| G2 | 38574 Leishmania | 8 | 0 | 0 | 0 |
| | donovani species complex | | | | |
| S | 5671 Leishmania infantum | 3 | 3 | 0 | 0 |
| S | 5661 Leishmania | 3 | 3 | 0 | 0 |
| | donovani | | | | |
| G2 | 38581 Leishmania major | 6 | 0 | 0 | 0 |
| | species complex | | | | |
| S | 5664 Leishmania major | 6 | 6\ | 0 | 0 |
| G2 | 38582 Leishmania | 3 | 0 | 0 | 0 |
| | mexicana species complex | | | | |
| S | 5665 Leishmania | 3 | 3 | 0 | 0 |
| | mexicana | | | | |
| G1 | 37616 Viannia | 5 | 5 | 0 | 0 |
| G | 5683 Leptomonas | 1 | 0 | 0 | 0 |
| S | 157538 Leptomonas | 1 | 1 | 0 | 0 |
| | pyrrhocoris | | | | |
| G | 5690 Trypanosoma | 39 | 0 | 0 | 0 |
| G1 | 47570 Schizotrypanum | 33 | 0 | 0 | 0 |
| S | 5693 Trypanosoma cruzi | 33 | 33 | 0 | 0 |
| G1 | 669453 Trypanosoma with | 3 | 0 | 0 | 0 |
| | unspecified subgenus | | | | |
| S | 71804 Trypanosoma grayi | 3 | 3 | 0 | 0 |
| G1 | 39700 Trypanozoon | 3 | 0 | 0 | 0 |
| S | 5691 Trypanosoma brucei | 3 | 3 | 0 | 0 |
| D1 | 2830 Haptophyceae | 30 | 0 | 0 | 0 |
| 0 | 73020 Isochrysidales | 30 | 0 | 0 | 0 |
| F | 418966 Noelaerhabdaceae | 30 | 0 | 0 | 0 |
| G | 2902 Emiliania | 30 | 0 | 0 | 0 |
| S | 2903 Emiliania huxleyi | 30 | 30 | 0 | 0 |
| C | 3027 Cryptophyta | 12 | 0 | 0 | 0 |
| 0 | 589342 Pyrenomonadales | 12 | 0 | 0 | 0 |
| F | 589343 Geminigeraceae | 12 | 0 | 0 | 0 |
| G | 55528 Guillardia | 12 | 0 | 0 | 0 |
| S | 55529 Guillardia theta | 12 | 12 | 0 | 0 |
| D1 | 554296 Apusozoa | 11 | 0 | 0 | 0 |
| F | 172820 Apusomonadidae | 11 | 0 | 0 | 0 |
| G | 877559 Thecamonas | 11 | 0 | 0 | 0 |
| S | 529818 Thecamonas | 11 | 11 | 0 | 0 |
| | trahens | | | | |
| D1 | 5719 Parabasalia | 2 | 0 | 0 | 0 |
| 0 | 37104 Trichomonadida | 2 | 0 | 0 | 0 |
| F | 181550 Trichomonadidae | 2 | 0 | 0 | 0 |
| G | 5721 Trichomonas | 2 | 0 | 0 | 0 |
| S | 5722 Trichomonas | 2 | 2 | 0 | 0 |
| | vaginalis | | | | |
| D1 | 207245 Fornicata | 1 | 0 | 0 | 0 |
| D2 | 5738 Diplomonadida | 1 | 0 | 0 | 0 |
| F | 5739 Hexamitidae | 1 | 0 | 0 | 0 |
| F1 | 68459 Giardiinae | 1 | 0 | 0 | 0 |
| G | 5740 Giardia | 1 | 0 | 0 | 0 |
| S | 5741 Giardia intestinalis | 1 | 1 | 0 | 0 |

TABLE 10
Report of the Abundance of Viruses

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| D | 10239 Viruses | 225 | 0 | 0 | 0 |
| D1 | 35268 Retro-transcribing viruses | 175 | 0 | 0 | 0 |
| F | 11632 Retroviridae | 175 | 0 | 0 | 0 |
| F1 | 327045 Orthoretrovirinae | 175 | 0 | 0 | 0 |
| G | 153135 Gammaretrovirus | 175 | 0 | 0 | 0 |
| G1 | 353769 unclassified | 141 | 0 | 0 | 0 |
| | Gammaretrovirus | | | | |
| S | 590745 Mus musculus mobilized | 86 | 86 | 0 | 0 |
| | endogenous polytropic provirus | | | | |
| S | 11819 Spleen focus-forming virus | 37 | 37 | 0 | 0 |
| S | 11830 Murine osteosarcoma virus | 12 | 12 | 0 | 0 |
| S | 99182 Murine leukemia-related | 3 | 3 | 0 | 0 |
| | retroviruses | | | | |
| S | 11788 Abelson murine leukemia | 3 | 3 | 0 | 0 |
| | virus | | | | |
| G1 | 353212 Mammalian | 33 | 0 | 0 | 0 |
| | gammaretrovirus group | | | | |
| G2 | 353210 Replication competent | 33 | 0 | 0 | 0 |
| | viruses | | | | |
| S | 11786 Murine leukemia virus | 33 | 33 | 0 | 0 |
| D1 | 35237 dsDNA viruses, no RNA | 48 | 0 | 0 | 0 |
| | stage | | | | |
| 0 | 28883 Caudovirales | 40 | 0 | 0 | 0 |
| F | 10699 Siphoviridae | 38 | 0 | 0 | 0 |
| F1 | 196894 unclassified Siphoviridae | 13 | 0 | 0 | 0 |
| S | 1385385 Streptococcus phage TP- | 6 | 6 | 0 | 0 |
| | 778L | | | | |
| S | 646413 Streptococcus phage 5093 | 4 | 4 | 0 | 0 |
| S | 1814960 Streptococcus virus 9874 | 1 | 1 | 0 | 0 |
| S | 1541819 Mycobacterium phage | 1 | 1 | 0 | 0 |
| | Omnicron | | | | |
| S | 1150990 Enterobacter phage Enc34 | 1 | 1 | 0 | 0 |
| G | 1623303 Sfillvirus | 11 | 0 | 0 | 0 |
| S | 682370 Streptococcus phage Alq132 | 6 | 6 | 0 | 0 |
| S | 306323 Streptococcus phage 2972 | 1 | 1 | 0 | 0 |
| S | 85154 Streptococcus phage 01205 | 1 | 1 | 0 | 0 |
| G | 1623304 Sfi21dtlvirus | 8 | 0 | 0 | 0 |
| S | 90410 Streptococcus phage DT1 | 5 | 5 | 0 | 0 |
| S | 112023 Streptococcus phage 7201 | 2 | 2 | 0 | 0 |
| S | 64186 Streptococcus phage Sfi21 | 1 | 1 | 0 | 0 |
| G | 186764 L5virus | 2 | 0 | 0 | 0 |
| S | 2003504 Mycobacterium virus | 2 | 2 | 0 | 0 |
| | Redrock | | | | |
| F | 10662 Myoviridae | 2 | 0 | 0 | 0 |
| F1 | 1198136 Tevenvirinae | 2 | 0 | 0 | 0 |
| G | 1913650 Cc3lvirus | 2 | 0 | 0 | 0 |
| S | 1913655 Enterobacter virus PG7 | 2 | 2 | 0 | 0 |
| F | 549779 Mimiviridae | 5 | 0 | 0 | 0 |
| F1 | 985780 unclassified Mimiviridae | 5 | 0 | 0 | 0 |
| S | 1094892 Megavirus chiliensis | 5 | 5 | 0 | 0 |
| D2 | 51368 unclassified dsDNA viruses | 2 | 0 | 0 | 0 |
| S | 1349409 Pandoravirus dulcis | 2 | 2 | 0 | 0 |
| F | 10240 Poxviridae | 1 | 0 | 0 | 0 |
| F1 | 10284 Entomopoxvirinae | 1 | 0 | 0 | 0 |
| G | 10286 Betaentomopoxvirus | 1 | 0 | 0 | 0 |
| S | 1179664 Mythimna separata | 1 | 1 | 0 | 0 |
| | entomopoxvirus | | | | |
| D1 | 29258 ssDNA viruses | 2 | 0 | 0 | 0 |
| F | 10841 Microviridae | 2 | 0 | 0 | 0 |
| F1 | 117574 unclassified Microviridae | 2 | 0 | 0 | 0 |
| S | 1655645 Parabacteroides phage YZ- | 2 | 2 | 0 | 0 |
| | 2015b | | | | |

TABLE 11
Report of the Abundance of Bacteria

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| D | 2 Bacteria | 10657882 | 0 | 78.03 | 99.86 |
| D1 | 1783270 FCB group | 6305012 | 0 | 46.16 | 59.08 |
| D2 | 68336 Bacteroidetes/Chlorobi group | 6304703 | 0 | 46.16 | 59.07 |
| P | 976 Bacteroidetes | 6304491 | 0 | 46.16 | 59.07 |
| C | 200643 Bacteroidia | 6300451 | 0 | 46.13 | 59.03 |
| 0 | 171549 Bacteroidales | 6300181 | 0 | 46.12 | 59.03 |
| F | 815 Bacteroidaceae | 3912934 | 0 | 28.65 | 36.66 |
| G | 816 Bacteroides | 3911586 | 0 | 28.64 | 36.65 |
| S | 46506 Bacteroides stercoris | 656815 | 656815 | 4.81 | 6.15 |
| S | 820 Bacteroides uniformis | 1398898 | 1398898 | 10.24 | 13.11 |
| S | 821 Bacteroides vulgatus | 570952 | 570952 | 4.18 | 5.35 |
| S | 28116 Bacteroides ovatus | 376631 | 376631 | 2.76 | 3.53 |
| S | 47678 Bacteroides caccae | 107493 | 107493 | 0.79 | 1.01 |
| S | 817 Bacteroides fragilis | 308172 | 308172 | 2.26 | 2.89 |
| S | 818 Bacteroides thetaiotaomicron | 42205 | 42205 | 0.31 | 0.4 |
| S | 291645 Bacteroides nordii | 28552 | 28552 | 0.21 | 0.27 |
| S | 310298 Bacteroides coprocola | 16870 | 16870 | 0.12 | 0.16 |
| S | 585543 Bacteroides sp. D20 | 43700 | 43700 | 0.32 | 0.41 |
| S | 457394 Bacteroides sp. 4_3_47FAA | 82836 | 82836 | 0.61 | 0.78 |
| S | 626929 Bacteroides clarus | 7860 | 7860 | 0.06 | 0.07 |
| S | 1078089 Bacteroides sp. HPS0048 | 8594 | 8594 | 0.06 | 0.08 |
| S | 626930 Bacteroides fluxus | 4981 | 4981 | 0.04 | 0.05 |
| S | 469590 Bacteroides sp. 2_2_4 | 15107 | 15107 | 0.11 | 0.14 |
| S | 469593 Bacteroides sp. 3_1_40A | 32544 | 32544 | 0.24 | 0.3 |
| S | 371601 Bacteroides xylanisolvens | 10839 | 10839 | 0.08 | 0.1 |
| S | 338188 Bacteroides finegoldii | 4870 | 4870 | 0.04 | 0.05 |
| S | 357276 Bacteroides dorei | 26184 | 26184 | 0.19 | 0.25 |
| S | 1965580 Bacteroides sp. An19 | 5992 | 5992 | 0.04 | 0.06 |
| S | 469592 Bacteroides sp. 3_1_19 | 19051 | 19051 | 0.14 | 0.18 |
| S | 28111 Bacteroides eggerthii | 3704 | 3704 | 0.03 | 0.03 |
| S | 457393 Bacteroides sp. 4_1_36 | 19511 | 19511 | 0.14 | 0.18 |
| S | 329854 Bacteroides intestinalis | 3505 | 3505 | 0.03 | 0.03 |
| S | 204516 Bacteroides massiliensis | 2302 | 2302 | 0.02 | 0.02 |
| S | 291644 Bacteroides salyersiae | 2255 | 2255 | 0.02 | 0.02 |
| S | 376806 Bacteroides gallinarum | 1984 | 1984 | 0.01 | 0.02 |
| S | 626931 Bacteroides oleiciplenus | 1849 | 1849 | 0.01 | 0.02 |
| S | 246787 Bacteroides cellulosilyticus | 4967 | 4967 | 0.04 | 0.05 |
| S | 1965632 Bacteroides sp. An322 | 1640 | 1640 | 0.01 | 0.02 |
| S | 671267 Bacteroides sartorii | 1666 | 1666 | 0.01 | 0.02 |
| S | 1796613 Bacteroides caecimuris | 1556 | 1556 | 0.01 | 0.01 |
| S | 1739298 Bacteroides sp. HMSC067B03 | 3868 | 3868 | 0.03 | 0.04 |
| S | 457387 Bacteroides sp. 1_1_30 | 4310 | 4310 | 0.03 | 0.04 |
| S | 457389 Bacteroides sp. 3_1_13 | 2601 | 2601 | 0.02 | 0.02 |
| S | 457395 Bacteroides sp. 9_1_42FAA | 5579 | 5579 | 0.04 | 0.05 |
| S | 290053 Bacteroides helcogenes | 1135 | 1135 | 0.01 | 0.01 |
| S | 469586 Bacteroides sp. 1_1_6 | 4881 | 4881 | 0.04 | 0.05 |
| S | 1903262 Bacteroides sp. Marseille-P3108 | 957 | 957 | 0.01 | 0.01 |
| S | 585544 Bacteroides sp. D22 | 2065 | 2065 | 0.02 | 0.02 |
| S | 457391 Bacteroides sp. 3_1_33FAA | 3170 | 3170 | 0.02 | 0.03 |
| S | 871324 Bacteroides stercorirosoris | 826 | 826 | 0.01 | 0.01 |
| S | 457390 Bacteroides sp. 3_1_23 | 1886 | 1886 | 0.01 | 0.02 |
| S | 1841855 Bacteroides sp. Marseille-P2653 | 881 | 881 | 0.01 | 0.01 |
| S | 1470345 Bacteroides timonensis | 794 | 794 | 0.01 | 0.01 |
| S | 1917883 Bacteroides sp. Marseille-P3166 | 804 | 804 | 0.01 | 0.01 |
| S | 556259 Bacteroides sp. D2 | 1053 | 1053 | 0.01 | 0.01 |
| S | 376805 Bacteroides salanitronis | 740 | 740 | 0.01 | 0.01 |
| S | 310300 Bacteroides pyogenes | 672 | 672 | 0 | 0.01 |
| S | 469589 Bacteroides sp. 2_1_33B | 2150 | 2150 | 0.02 | 0.02 |
| S | 387090 Bacteroides coprophilus | 581 | 581 | 0 | 0.01 |
| S | 1965640 Bacteroides sp. An51A | 852 | 852 | 0.01 | 0.01 |
| S | 1965613 Bacteroides sp. An269 | 742 | 742 | 0.01 | 0.01 |
| S | 1841856 Bacteroides mediterraneensis | 476 | 476 | 0 | 0 |
| S | 376804 Bacteroides barnesiae | 423 | 423 | 0 | 0 |
| S | 1347393 Bacteroides neonati | 416 | 416 | 0 | 0 |
| S | 674529 Bacteroides faecis | 940 | 940 | 0.01 | 0.01 |
| S | 310297 Bacteroides plebeius | 421 | 421 | 0 | 0 |
| S | 1871006 Bacteroides sp. Marseille-P3132 | 387 | 387 | 0 | 0 |
| S | 85831 Bacteroides acidifaciens | 358 | 358 | 0 | 0 |
| S | 1133319 Bacteroides reticulotermitis | 261 | 261 | 0 | 0 |
| S | 871325 Bacteroides faecichinchillae | 264 | 264 | 0 | 0 |
| S | 556258 Bacteroides sp. DI. | 37178 | 37178 | 0.27 | 0.35 |
| S | 1163670 Bacteroides sp. 14(A) | 413 | 413 | 0 | 0 |
| S | 93974 Bacteroides sp. AR20 | 628 | 628 | 0 | 0.01 |
| S | 1907658 Bacteroides sp. Ma rseille-P3208T | 203 | 203 | 0 | 0 |
| S | 1739517 Bacteroides sp. HMSC073E02 | 2895 | 2895 | 0.02 | 0.03 |
| S | 1965620 Bacteroides sp. An279 | 251 | 251 | 0 | 0 |
| S | 469587 Bacteroides sp. 2_1_16 | 1052 | 1052 | 0.01 | 0.01 |
| S | 477666 Bacteroides graminisolvens | 113 | 113 | 0 | 0 |
| S | 1739319 Bacteroides sp. HMSC068A09 | 899 | 899 | 0.01 | 0.01 |
| S | 457392 Bacteroides sp. 3_2_5 | 5121 | 5121 | 0.04 | 0.05 |
| S | 732242 Bacteroides paurosaccharolyticus | 51 | 51 | 0 | 0 |
| S | 1297750 Bacteroides Iuti | 29 | 29 | 0 | 0 |
| S | 1852362 Bacteroides ihuae | 28 | 28 | 0 | 0 |
| S | 151276 Bacteroides coprosuis | 24 | 24 | 0 | 0 |
| S | 392838 Bacteroides propionicifaciens | 13 | 13 | 0 | 0 |
| S | 469588 Bacteroides sp. 2_1_22 | 4074 | 4074 | 0.03 | 0.04 |
| S | 1450439 Bacteroides sp. UW | 1 | 1 | 0 | 0 |
| G | 1926659 Mediterranea | 1302 | 0 | 0.01 | 0.01 |
| S | 1965586 Mediterranea sp. An20 | 762 | 762 | 0.01 | 0.01 |
| S | 1841865 Mediterranea massiliensis | 539 | 539 | 0 | 0.01 |
| G | 118966 Anaerorhabdus | 46 | 0 | 0 | 0 |
| S | 118967 Anaerorhabdus furcosa | 46 | 46 | 0 | 0 |
| F | 171550 Rikenellaceae | 1705527 | 0 | 12.49 | 15.98 |
| G | 239759 Alistipes | 1703819 | 0 | 12.47 | 15.96 |
| S | 214856 Alistipes finegoldii | 683209 | 683209 | 5 | 6.4 |
| S | 28117 Alistipes putredinis | 291675 | 291675 | 2.14 | 2.73 |
| S | 328814 Alistipes shahli | 246655 | 246655 | 1.81 | 2.31 |
| S | 328813 Alistipes onderdonkii | 196619 | 196619 | 1.44 | 1.84 |
| S | 908612 Alistipes sp. HGB5 | 119109 | 119109 | 0.87 | 1.12 |
| S | 1118061 Alistipes obesi | 45000 | 45000 | 0.33 | 0.42 |
| S | 1470347 Alistipes ihumil | 32731 | 32731 | 0.24 | 0.31 |
| S | 1499682 Alistipes sp. AL-1 | 53142 | 53142 | 0.39 | 0.5 |
| S | 1288121 Alistipes senegalensis | 14730 | 14730 | 0.11 | 0.14 |
| S | 1780376 Alistipes sp. CHKCI003 | 6434 | 6434 | 0.05 | 0.06 |
| S | 626932 Alistipes indistinctus | 5033 | 5033 | 0.04 | 0.05 |
| S | 1465754 Alistipes timonensis | 3358 | 3358 | 0.02 | 0.03 |
| S | 1816676 Alistipes sp. Marseille-P2431 | 2547 | 2547 | 0.02 | 0.02 |
| S | 1965650 Alistipes sp. An66 | 1256 | 1256 | 0.01 | 0.01 |
| S | 1965546 Alistipes sp. An116 | 863 | 863 | 0.01 | 0.01 |
| S | 1965631 Alistipes sp. An31A | 707 | 707 | 0.01 | 0.01 |
| S | 1965645 Alistipes sp. An54 | 738 | 738 | 0.01 | 0.01 |
| S | 1339253 Alistipes sp. ZOR0009 | 4 | 4 | 0 | 0 |
| S | 1501391 Alistipes inops | 3 | 3 | 0 | 0 |
| G | 28138 Rikenella | 1091 | 0 | 0.01 | 0.01 |
| S | 28139 Rikenella microfusus | 1091 | 1091 | 0.01 | 0.01 |
| G | 1929083 Tidjanibacter | 575 | 0 | 0 | 0.01 |
| S | 1871003 Tidjanibacter massiliensis | 575 | 575 | 0 | 0.01 |
| G | 1980038 Millionella | 23 | 0 | 0 | 0 |
| S | 1871023 Millionella massiliensis | 23 | 23 | 0 | 0 |
| G | 1611681 Mucinivorans | 18 | 0 | 0 | 0 |
| S | 1433126 Mucinivorans hirudinis | 18 | 18 | 0 | 0 |
| F | 2005525 Tannerellaceae | 565710 | 0 | 4.14 | 5.3 |
| G | 375288 Parabacteroides | 536043 | 0 | 3.92 | 5.02 |
| S | 46503 Parabacteroides merdae | 244990 | 244990 | 1.79 | 2.3 |
| S | 328812 Parabacteroides goldsteinii | 54004 | 54004 | 0.4 | 0.51 |
| S | 823 Parabacteroides distasonis | 113923 | 113923 | 0.83 | 1.07 |
| S | 658662 Parabacteroides sp. D26 | 40182 | 40182 | 0.29 | 0.38 |
| S | 387661 Parabacteroides johnsonii | 7730 | 7730 | 0.06 | 0.07 |
| S | 574930 Parabacteroides gordonii | 2560 | 2560 | 0.02 | 0.02 |
| S | 469591 Parabacteroides sp. 20_3 | 44426 | 44426 | 0.33 | 0.42 |
| S | 1750560 Parabacteroides sp. 5N4 | 2065 | 2065 | 0.02 | 0.02 |
| S | 563193 Parabacteroides sp. D13 | 5371 | 5371 | 0.04 | 0.05 |
| S | 1078087 Parabacteroides sp. HG50025 | 967 | 967 | 0.01 | 0.01 |
| S | 1871013 Parabacteroides sp. Marseille-P3236 | 653 | 653 | 0 | 0.01 |
| S | 2025876 Parabacteroides sp. CT06 | 10490 | 10490 | 0.08 | 0.1 |
| S | 2025877 Parabacteroides sp. AT13 | 2749 | 2749 | 0.02 | 0.03 |
| S | 1965619 Parabacteroides sp. An277 | 43 | 43 | 0 | 0 |
| S | 1235803 Parabacteroides sp. A5F519 | 5854 | 5854 | 0.04 | 0.05 |
| 5 | 1917887 Parabacteroides sp. Marseille-P3160 | 12 | 12 | 0 | 0 |
| 5 | 1037355 Parabacteroides chartae | 12 | 12 | 0 | 0 |
| 5 | 658661 Parabacteroides sp. D25 | 2 | 2 | 0 | 0 |
| 5 | 457388 Parabacteroides sp. 2_1_7 | 2 | 2 | 0 | 0 |
| G | 195950 Tannerella | 29666 | 0 | 0.22 | 0.28 |
| 5 | 665949 Tannerella sp. 6_1_58FAA_CT1 | 29336 | 29336 | 0.21 | 0.27 |
| 5 | 28112 Tannerella forsythia | 221 | 221 | 0 | 0 |
| 5 | 712710 Tannerella sp. oral taxon HOT-286 | 108 | 108 | 0 | 0 |
| F | 1853231 Odoribacteraceae | 64842 | 0 | 0.47 | 0.61 |
| G | 283168 Odoribacter | 63991 | 0 | 0.47 | 0.6 |
| 5 | 28118 Odoribacter splanchnicus | 63959 | 63959 | 0.47 | 0.6 |
| 5 | 626933 Odoribacter laneus | 32 | 32 | 0 | 0 |
| G | 574697 Butyricimonas | 769 | 0 | 0.01 | 0.01 |
| 5 | 544645 Butyricimonas virosa | 412 | 412 | 0 | 0 |
| S | 1816677 Butyricimonas sp. Marseille-P2440 | 163 | 163 | 0 | 0 |
| S | 544644 Butyricimonas synergistica | 153 | 153 | 0 | 0 |
| S | 1965649 Butyricimonas sp. An62 | 40 | 40 | 0 | 0 |
| G | 1926651 Culturomica | 81 | 0 | 0 | 0 |
| S | 1841857 Culturomica massiliensis | 81 | 81 | 0 | 0 |
| F | 171552 Prevotellaceae | 23830 | 0 | 0.17 | 0.22 |
| G | 838 Prevotella | 22658 | 0 | 0.17 | 0.21 |
| S | 419005 Prevotella amnli | 6970 | 6970 | 0.05 | 0.07 |
| S | 2024228 Prevotella sp. P4-51 | 7505 | 7505 | 0.05 | 0.07 |
| S | 165179 Prevotella copri | 1630 | 1630 | 0.01 | 0.02 |
| S | 655809 Prevotella sp. oral taxon 472 | 788 | 788 | 0.01 | 0.01 |
| S | 1676614 Prevotella sp. 109 | 608 | 608 | 0 | 0.01 |
| S | 1776379 Prevotella sp. KHD1 | 441 | 441 | 0 | 0 |
| S | 2022527 Prevotella sp. 885 | 276 | 276 | 0 | 0 |
| S | 1284775 Prevotella sp. 57-1-8 | 284 | 284 | 0 | 0 |
| S | 386414 Prevotella timonensis | 268 | 268 | 0 | 0 |
| S | 1287488 Prevotella sp. S7 MS 2 | 752 | 752 | 0.01 | 0.01 |
| S | 596085 Prevotella aurantiaca | 143 | 143 | 0 | 0 |
| S | 305719 Prevotella baroniae | 131 | 131 | 0 | 0 |
| S | 228604 Prevotella salivae | 116 | 116 | 0 | 0 |
| S | 1602172 Prevotella sp. P5-125 | 449 | 449 | 0 | 0 |
| S | 28125 Prevotella bivia | 218 | 218 | 0 | 0 |
| S | 28129 Prevotella denticola | 99 | 99 | 0 | 0 |
| S | 28131 Prevotella intermedia | 94 | 94 | 0 | 0 |
| S | 839 Prevotella ruminicola | 108 | 108 | 0 | 0 |
| S | 28128 Prevotella corporis | 83 | 83 | 0 | 0 |
| S | 77095 Prevotella bryantii | 65 | 65 | 0 | 0 |
| S | 1855325 Prevotella sp. KH2C16 | 69 | 69 | 0 | 0 |
| S | 1392486 Prevotella sp. HUN102 | 52 | 52 | 0 | 0 |
| S | 652716 Prevotella sp. oral taxon 299 | 50 | 50 | 0 | 0 |
| S | 340486 Prevotella conceptionensis | 68 | 68 | 0 | 0 |
| S | 470565 Prevotella histicola | 47 | 47 | 0 | 0 |
| S | 652721 Prevotella sp. oral taxon 317 | 53 | 53 | 0 | 0 |
| S | 28127 Prevotella buccalis | 37 | 37 | 0 | 0 |
| S | 28132 Prevotella melaninogenica | 48 | 48 | 0 | 0 |
| S | 242750 Prevotella bergensis | 33 | 33 | 0 | 0 |
| S | 589436 Prevotella fusca | 32 | 32 | 0 | 0 |
| S | 363265 Prevotella stercorea | 32 | 32 | 0 | 0 |
| S | 2024216 Prevotella sp. P5-126 | 139 | 139 | 0 | 0 |
| S | 2024222 Prevotella sp. P5-92 | 28 | 28 | 0 | 0 |
| S | 2024219 Prevotella sp. P4-98 | 139 | 139 | 0 | 0 |
| S | 189722 Prevotella marshii | 31 | 31 | 0 | 0 |
| S | 1433844 Prevotella sp. HJM029 | 31 | 31 | 0 | 0 |
| S | 28130 Prevotella disiens | 35 | 35 | 0 | 0 |
| S | 439703 Prevotella maculosa | 25 | 25 | 0 | 0 |
| S | 425941 Prevotella nanceiensis | 22 | 22 | 0 | 0 |
| S | 563031 Prevotella sp. C561 | 27 | 27 | 0 | 0 |
| S | 28133 Prevotella nigrescens | 30 | 30 | 0 | 0 |
| S | 1761885 Prevotella sp. khp7 | 47 | 47 | 0 | 0 |
| S | 1602168 Prevotella sp. P4-65 | 81 | 81 | 0 | 0 |
| S | 1161412 Prevotella sp. ICM33 | 26 | 26 | 0 | 0 |
| S | 83231 Prevotella brevis | 17 | 17 | 0 | 0 |
| S | 185294 Prevotella paludivivens | 15 | 15 | 0 | 0 |
| S | 2024223 Prevotella sp. P3-122 | 15 | 15 | 0 | 0 |
| S | 1227276 Prevotella sp. F0091 | 16 | 16 | 0 | 0 |
| S | 515414 Prevotella falsenii | 14 | 14 | 0 | 0 |
| S | 282402 Prevotella multiformis | 16 | 16 | 0 | 0 |
| S | 407975 Prevotella pleuritidis | 17 | 17 | 0 | 0 |
| S | 2024224 Prevotella sp. P2-180 | 12 | 12 | 0 | 0 |
| S | 1761888 Prevotella sp. tc2-28 | 12 | 12 | 0 | 0 |
| S | 1032506 Prevotella sp. MSX73 | 39 | 39 | 0 | 0 |
| S | 310514 Prevotella multisaccharivorax | 12 | 12 | 0 | 0 |
| S | 28136 Prevotella oulorum | 13 | 13 | 0 | 0 |
| S | 76123 Prevotella enoeca | 14 | 14 | 0 | 0 |
| S | 1280674 Prevotella sp. AGR2160 | 10 | 10 | 0 | 0 |
| S | 712461 Prevotella sp. oral taxon 306 | 12 | 12 | 0 | 0 |
| S | 28135 Prevotella oris | 14 | 14 | 0 | 0 |
| S | 840 Prevotella loescheil | 12 | 12 | 0 | 0 |
| S | 1917878 Prevotella ihu mil | 9 | 9 | 0 | 0 |
| S | 589437 Prevotella scopos | 9 | 9 | 0 | 0 |
| S | 28126 Prevotella buccae | 9 | 9 | 0 | 0 |
| S | 1761889 Prevotella sp. tf2-5 | 8 | 8 | 0 | 0 |
| S | 185293 Prevotella oryzae | 8 | 8 | 0 | 0 |
| S | 28137 Prevotella veroralis | 8 | 8 | 0 | 0 |
| S | 1761887 Prevotella sp. ne3005 | 7 | 7 | 0 | 0 |
| S | 1158294 Prevotella sp. 10(H) | 7 | 7 | 0 | 0 |
| S | 633701 Prevotella saccharolytica | 7 | 7 | 0 | 0 |
| S | 60133 Prevotella pallens | 7 | 7 | 0 | 0 |
| S | 52229 [Melia] seregens | 7 | 7 | 0 | 0 |
| S | 1111130 Prevotella sp. BV3P1 | 6 | 6 | 0 | 0 |
| S | 189723 Prevotella micans | 6 | 6 | 0 | 0 |
| S | 2024225 Prevotella sp. P5-108 | 5 | 5 | 0 | 0 |
| S | 77768 Prevotella albensis | 5 | 5 | 0 | 0 |
| S | 2024226 Prevotella sp. P5-64 | 4 | 4 | 0 | 0 |
| S | 2024218 Prevotella sp. P4-119 | 4 | 4 | 0 | 0 |
| S | 1602169 Prevotella sp. P4-76 | 4 | 4 | 0 | 0 |
| S | 1410613 Prevotella sp. P6B1 | 4 | 4 | 0 | 0 |
| S | 28134 Prevotella oralis | 4 | 4 | 0 | 0 |
| S | 1761884 Prevotella sp. khpl | 3 | 3 | 0 | 0 |
| S | 1408310 Prevotella sp. MA2016 | 3 | 3 | 0 | 0 |
| S | 589537 Prevotella dentasini | 3 | 3 | 0 | 0 |
| S | 228603 Prevotella shahii | 3 | 3 | 0 | 0 |
| S | 52227 Prevotella dentalis | 3 | 3 | 0 | 0 |
| S | 2024217 Prevotella sp. P5-50 | 2 | 2 | 0 | 0 |
| S | 1739496 Prevotella sp. HMSC069G02 | 2 | 2 | 0 | 0 |
| S | 1384078 Prevotella sp. DNF00663 | 2 | 2 | 0 | 0 |
| S | 645274 Prevotella sp. BP1-148 | 2 | 2 | 0 | 0 |
| S | 645273 Prevotella sp. BP1-145 | 2 | 2 | 0 | 0 |
| S | 2024227 Prevotella sp. P4-67 | 1 | 1 | 0 | 0 |
| S | 2024221 Prevotella sp. P3-92 | 1 | 1 | 0 | 0 |
| S | 1761886 Prevotella sp. Ic2012 | 1 | 1 | 0 | 0 |
| S | 1739459 Prevotella sp. HMSC073D09 | 1 | 1 | 0 | 0 |
| S | 1602171 Prevotella sp. P5-119 | 1 | 1 | 0 | 0 |
| S | 1602170 Prevotella sp. P5-60 | 1 | 1 | 0 | 0 |
| S | 81582 Prevotella aff. ruminicola Tc2-24 | 1 | 1 | 0 | 0 |
| G | 577309 Paraprevotella | 956 | 0 | 0.01 | 0.01 |
| S | 454155 Paraprevotella xylaniphila | 572 | 572 | 0 | 0.01 |
| S | 454154 Paraprevotella clara | 383 | 383 | 0 | 0 |
| G | 1283313 Alloprevotella | 144 | 0 | 0 | 0 |
| S | 76122 Alloprevotella tannerae | 139 | 139 | 0 | 0 |
| S | 671218 Alloprevotella rava | 3 | 3 | 0 | 0 |
| S | 712469 Prevotella sp. oral taxon 473 | 2 | 2 | 0 | 0 |
| G | 1926672 Prevotellamassilia | 51 | 0 | 0 | 0 |
| S | 1852370 Prevotellamassilia timonensis | 51 | 51 | 0 | 0 |
| F1 | 224765 unclassified Prevotellaceae | 18 | 0 | 0 | 0 |
| S | 1520830 Prevotellaceae bacterium HUN156 | 8 | 8 | 0 | 0 |
| S | 1945887 Prevotellaceae bacterium MN60 | 5 | 5 | 0 | 0 |
| S | 1852368 Prevotellaceae bacterium Marseille-P2826 | 3 | 3 | 0 | 0 |
| S | 1945886 Prevotellaceae bacterium KH2P17 | 1 | 1 | 0 | 0 |
| F | 2005519 Barnesiellaceae | 25602 | 0 | 0.19 | 0.24 |
| G | 1348911 Coprobacter | 24652 | 0 | 0.18 | 0.23 |
| S | 1099853 Coprobacter fastidiosus | 23908 | 23908 | 0.18 | 0.22 |
| S | 1501392 Coprobacter secundus | 743 | 743 | 0.01 | 0.01 |
| G | 397864 Barnesiella | 949 | 0 | 0.01 | 0.01 |
| S | 487174 Barnesiella intestinihominis | 335 | 335 | 0 | 0 |
| S | 1965590 Barnesiella sp. An22 | 280 | 280 | 0 | 0 |
| S | 1965646 Barnesiella sp. An55 | 189 | 189 | 0 | 0 |
| S | 397865 Barnesiella viscericola | 144 | 144 | 0 | 0 |
| F | 171551 Porphyromonadaceae | 1240 | 0 | 0.01 | 0.01 |
| G | 1635148 Sanguibacteroides | 499 | 0 | 0 | 0 |
| S | 1547597 Sanguibacteroides justesenii | 499 | 499 | 0 | 0 |
| G | 836 Porphyromonas | 438 | 0 | 0 | 0 |
| S | 1537504 Porphyromonas sp. COT-108 OH1349 | 111 | 111 | 0 | 0 |
| S | 28123 Porphyromonas asaccharolytica | 60 | 60 | 0 | 0 |
| S | 837 Porphyromonas gingivalis | 48 | 48 | 0 | 0 |
| S | 28115 Porphyromonas macacae | 38 | 38 | 0 | 0 |
| S | 28124 Porphyromonas endodontalis | 33 | 33 | 0 | 0 |
| S | 111105 Porphyromonas gulae | 33 | 33 | 0 | |
| S | 281920 Porphyromonas uenonis | 18 | 18 | 0 | 0 |
| S | 36874 Porphyromonas cangingivalis | 18 | 18 | 0 | 0 |
| S | 501496 Porphyromonas bennonis | 15 | 15 | 0 | 0 |
| S | 1515613 Porphyromonas sp. COT-239 OH1446 | 9 | 9 | 0 | 0 |
| S | 1739394 Porphyromonas sp. HMSC065F10 | 8 | 8 | 0 | 0 |
| S | 1515614 Porphyromonas sp. COT-108 OH2963 | 8 | 8 | 0 | 0 |
| S | 28114 Porphyromonas levii | 8 | 8 | 0 | 0 |
| S | 41976 Porphyromonas catoniae | 7 | 7 | 0 | 0 |
| S | 393921 Porphyromonas crevioricanis | 5 | 5 | 0 | 0 |
| S | 266762 Porphyromonas gingivicanis | 3 | 3 | 0 | 0 |
| S | 29524 Porphyromonas circumdentaria | 3 | 3 | 0 | 0 |
| S | 712437 Porphyromonas sp. oral taxon 278 | 2 | 2 | 0 | 0 |
| S | 322095 Porphyromonas somerae | 2 | 2 | 0 | 0 |
| S | 36875 Porphyromonas canoris | 2 | 2 | 0 | 0 |
| S | 1739529 Porphyromonas sp. HMSC077F02 | 1 | 1 | 0 | 0 |
| S | 1515618 Porphyromonas sp. COT-052 OH4946 | 1 | 1 | 0 | 0 |
| S | 1515617 Porphyromonas sp. COT-290 OH3588 | 1 | 1 | 0 | 0 |
| S | 997829 Porphyromonas sp. KLE 1280 | 1 | 1 | 0 | 0 |
| G | 1923867 Gabonia | 162 | 0 | 0 | 0 |
| S | 1686296 Gabonia massiliensis | 162 | 162 | 0 | 0 |
| G | 1911312 Gabonibacter | 66 | 0 | 0 | 0 |
| S | 1720195 Gabonibacter massiliensis | 66 | 66 | 0 | 0 |
| F1 | 361210 unclassified Porphyromonadaceae | 28 | 0 | 0 | 0 |
| S | 1720317 Porphyromonadaceae bacterium FC4 | 14 | 14 | 0 | 0 |
| S | 1658779 Porphyromonadaceae bacterium H1 | 6 | 6 | 0 | 0 |
| S | 1517682 Porphyromonadaceae bacterium | 6 | 6 | 0 | 0 |
| | COT-184 OH4590 | | | | |
| S | 1855399 Porphyromonadaceae bacterium | 1 | 1 | 0 | 0 |
| | NLAE-zl-C104 | | | | |
| S | 1855397 Porphyromonadaceae bacterium | 1 | 1 | 0 | 0 |
| | KH3R12 | | | | |
| G | 2034765 Massilibacteroides | 26 | 0 | 0 | 0 |
| S | 1673718 Massilibacteroides vaginae | 26 | 26 | 0 | 0 |
| G | 307628 Petrimonas | 16 | 0 | 0 | 0 |
| S | 1642646 Petrimonas mucosa | 16 | 16 | 0 | 0 |
| G | 1924067 Lascolabacillus | 2 | 0 | 0 | 0 |
| S | 1627894 Lascolabacillus massiliensis | 2 | 2 | 0 | 0 |
| G | 1159323 Macellibacteroides | 1 | 0 | 0 | 0 |
| S | 1857568 Macellibacteroides sp. HH-ZS | 1 | 1 | 0 | 0 |
| 01 | 333046 unclassified Bacteroidales | 235 | 0 | 0 | 0 |
| 02 | 185291 unclassified Bacteroidales (miscellaneous) | 227 | 0 | 0 | 0 |
| S | 1945890 Bacteroidales bacterium WCE2004 | 94 | 94 | 0 | 0 |
| S | 1497954 Bacteroidales bacterium KA00344 | 60 | 60 | 0 | 0 |
| S | 1855373 Bacteroidales bacterium KHT7 | 29 | 29 | 0 | 0 |
| S | 1945891 Bacteroidales bacterium WCE2008 | 12 | 12 | 0 | 0 |
| S | 1688776 Bacteroidales bacterium 6E | 9 | 9 | 0 | 0 |
| S | 1633202 Bacteroidales bacterium Barb6XT | 6 | 6 | 0 | 0 |
| S | 1400053 Bacteroidales bacterium CF | 6 | 6 | 0 | 0 |
| S | 1633201 Bacteroidales bacterium Barb6 | 5 | 5 | 0 | 0 |
| S | 1497953 Bacteroidales bacterium KA00251 | 5 | 5 | 0 | 0 |
| G | 467084 Candidatus Symbiothrix | 5 | 0 | 0 | 0 |
| S | 467085 Candidatus Symbiothrix | 5 | 5 | 0 | 0 |
| | dinenymphae | | | | |
| G | 511434 Candidatus Azobacteroides | 3 | 0 | 0 | 0 |
| S | 511435 Candidatus Azobacteroides | 3 | 3 | 0 | 0 |
| | pseudotrichonymphae | | | | |
| F | 2005473 Muribaculaceae | 127 | 0 | 0 | 0 |
| G | 1918540 Muribaculum | 127 | 127 | 0 | 0 |
| F | 2005520 Dysgonamonadaceae | 99 | 0 | 0 | 0 |
| G | 156973 Dysgonomonas | 96 | 0 | 0 | 0 |
| S | 156974 Dysgonomonas gadei | 64 | 64 | 0 | 0 |
| S | 1346286 Dysgonomonas macrotermitis | 17 | 17 | 0 | 0 |
| S | 1658009 Dysgonomonas sp. HGC4 | 7 | 7 | 0 | 0 |
| S | 1658008 Dysgonomonas sp. BGC7 | 4 | 4 | 0 | 0 |
| S | 163665 Dysgonomonas mossii | 2 | 2 | 0 | 0 |
| S | 45254 Dysgonomonas capnocytophagoides | 2 | 2 | 0 | 0 |
| G | 294702 Proteiniphilum | 3 | 0 | 0 | 0 |
| S | 1642647 Proteiniphilum saccharofermentans | 2 | 2 | 0 | 0 |
| S | 294710 Proteiniphilum acetatigenes | 1 | 1 | 0 | 0 |
| F | 2005523 Paludibacteraceae | 31 | 0 | 0 | 0 |
| G | 346096 Paludibacter | 31 | 0 | 0 | 0 |
| S | 681398 Paludibacterjiangxiensis | 20 | 20 | 0 | 0 |
| S | 185300 Paludibacter propionicigenes | 11 | 11 | 0 | 0 |
| F | 1840213 Lentimicrobiaceae | 2 | 0 | 0 | 0 |
| G | 1840214 Lentimicrobium | 2 | 0 | 0 | 0 |
| S | 1678841 Lentimicrobium saccharophilum | 2 | 2 | 0 | 0 |
| 0 | 1970189 Marinilabiliales | 269 | 0 | 0 | 0 |
| F | 1471398 Prolixibacteraceae | 117 | 0 | 0 | 0 |
| G | 314318 Prolixibacter | 64 | 0 | 0 | 0 |
| S | 314319 Prolixibacter bellarlivorans | 64 | 64 | 0 | 0 |
| G | 1254401 Sunxiuqinia | 26 | 0 | 0 | 0 |
| S | 1409788 Sunxiuqinia dokdonensis | 20 | 20 | 0 | 0 |
| S | 655355 Sunxiuqinia elliptica | 6 | 6 | 0 | 0 |
| G | 1471399 Draconibacterium | 14 | 0 | 0 | 0 |
| S | 1544798 Draconibacterium sediminis | 5 | 5 | 0 | 0 |
| S | 1168034 Draconibacterium orientale | 2 | 2 | 0 | 0 |
| G | 1573806 Mariniphaga | 10 | 0 | 0 | 0 |
| S | 1484053 Mariniphaga anaerophila | 10 | 10 | 0 | 0 |
| G | 1649501 Tangfeifania | 3 | 0 | 0 | 0 |
| S | 1168035 Tangfeifania diversioriginum | 3 | 3 | 0 | 0 |
| F | 558415 Marinilabiliaceae | 117 | 0 | 0 | 0 |
| G | 286729 Alkaliflexus | 39 | 0 | 0 | 0 |
| S | 286730 Alkaliflexus imshenetskii | 39 | 39 | 0 | 0 |
| G | 177399 Anaerophaga | 33 | 0 | 0 | 0 |
| S | 177400 Anaerophaga thermohalophila | 33 | 33 | 0 | 0 |
| G | 1849718 Labilibacter | 14 | 0 | 0 | 0 |
| S | 1849719 Labilibacter aurantiacus | 13 | 13 | 0 | 0 |
| S | 1477105 Labilibacter marinus | 1 | 1 | 0 | 0 |
| G | 1236988 Geofilum | 12 | 0 | 0 | 0 |
| S | 472113 Geofilum rubicundum | 10 | 10 | 0 | 0 |
| S | 1965019 Geofilum sp. H F401 | 2 | 2 | 0 | 0 |
| G | 1618113 Saccharicrinis | 6 | 0 | 0 | 0 |
| S | 982 Saccharicrinis fermentans | 6 | 6 | 0 | 0 |
| G | 59738 Marinilabilia | 6 | 0 | 0 | 0 |
| S | 989 Marinilabilia salmonicolor | 6 | 6 | 0 | 0 |
| G | 1193324 Alkalitalea - | 4 | 0 | 0 | 0 |
| S | 889453 Alkalitalea saponilacus | 4 | 4 | 0 | 0 |
| G | 1434049 Thermophagus | 2 | 0 | 0 | 0 |
| S | 385682 Thermophagus xiamenensis | 2 | 2 | 0 | 0 |
| F | 1970190 Salinivirgaceae | 33 | 0 | 0 | 0 |
| G | 1970191 Salinivirga | 33 | 0 | 0 | 0 |
| S | 1307839 Salinivirga cyanobacteriivorans | 33 | 33 | 0 | 0 |
| F | 1573805 Marinifilaceae | 2 | 0 | 0 | 0 |
| G | 866673 Marinifilum | 2 | 0 | 0 | 0 |
| S | 570161 Marinifilum fragile | 2 | 2 | 0 | 0 |
| C | 117743 Flavobacteriia | 2155 | 0 | 0.02 | 0.02 |
| 0 | 200644 Flavobacteriales | 2151 | 0 | 0.02 | 0.02 |
| F | 49546 Flavobacteriaceae | 2096 | 0 | 0.02 | 0.02 |
| G | 237 Flavobacterium | 557 | 0 | 0 | 0.01 |
| S | 55197 Flavobacterium branchiophilum | 81 | 81 | 0 | 0 |
| S | 1124188 Flavobacterium fontis | 69 | 69 | 0 | 0 |
| S | 996 Flavobacterium columnare | 49 | 49 | 0 | 0 |
| S | 498301 Flavobacterium rivuli | 41 | 41 | 0 | 0 |
| S | 1603293 Flavobacterium sp. 316 | 25 | 25 | 0 | 0 |
| 5 | 96345 Flavobacterium psychrophilum | 22 | 22 | 0 | 0 |
| 5 | 657326 Flavobacterium beibuense | 14 | 14 | 0 | 0 |
| 5 | 683124 Flavobacterium haoranii | 22 | 22 | 0 | 0 |
| 5 | 426226 Flavobacterium subsaxonicum | 15 | 15 | 0 | 0 |
| 5 | 1306519 Flavobacterium communis | 25 | 25 | 0 | 0 |
| 5 | 913024 Flavobacterium ummariense | 12 | 12 | 0 | 0 |
| 5 | 1736351 Flavobacterium sp. Leaf359 | 9 | 9 | 0 | 0 |
| 5 | 1114867 Flavobacterium plurextorum | 9 | 9 | 0 | 0 |
| 5 | 1202724 Flavobacterium akiainvivens | 8 | 8 | 0 | 0 |
| 5 | 362418 Flavobacterium reichenbachii | 8 | 8 | 0 | 0 |
| 5 | 350893 Flavobacterium daejeonense | 8 | 8 | 0 | 0 |
| 5 | 1736610 Flavobacterium sp. Root935 | 7 | 7 | 0 | 0 |
| 5 | 229205 Flavobacterium micromati | 7 | 7 | 0 | 0 |
| 5 | 1566022 Flavobacterium sp. JRM | 6 | 6 | 0 | 0 |
| 5 | 1144313 Flavobacterium sp. CF136 | 6 | 6 | 0 | 0 |
| 5 | 946678 Flavobacterium araucananum | 6 | 6 | 0 | 0 |
| 5 | 1298868 Flavobacterium sp. URHB0058 | 5 | 5 | 0 | 0 |
| 5 | 739143 Flavobacterium phragmitis | 5 | 5 | 0 | 0 |
| 5 | 986 Flavobacterium johnsoniae | 5 | 5 | 0 | 0 |
| 5 | 2022802 Flavobacterium sp. TH021 | 4 | 4 | 0 | 0 |
| 5 | 1938609 Flavobacterium sp. LM4 | 4 | 4 | 0 | 0 |
| 5 | 1929267 Flavobacterium sp. FPG59 | 4 | 4 | 0 | 0 |
| 5 | 1587524 Flavobacterium sp. MEB061 | 4 | 4 | 0 | 0 |
| 5 | 1299341 Flavobacterium urocaniciphilum | 4 | 4 | 0 | 0 |
| 5 | 312277 Flavobacterium indicum | 4 | 4 | 0 | 0 |
| 5 | 206041 Flavobacterium gelidilacus | 4 | 4 | 0 | 0 |
| 5 | 178356 Flavobacterium xinjiangense | 4 | 4 | 0 | 0 |
| 5 | 29536 Flavobacterium succinicans | 4 | 4 | 0 | 0 |
| 5 | 2023067 Flavobacterium sp. TH167 | 3 | 3 | 0 | 0 |
| 5 | 1763534 Flavobacterium crassostreae | 3 | 3 | 0 | 0 |
| 5 | 1492738 Flavobacterium seoulense | 3 | 3 | 0 | 0 |
| 5 | 415425 Flavobacterium terrae | 3 | 3 | 0 | 0 |
| 5 | 229204 Flavobacterium frigoris | 3 | 3 | 0 | 0 |
| 5 | 983 Flavobacterium flevense | 3 | 3 | 0 | 0 |
| 5 | 1736605 Flavobacterium sp. Root901 | 2 | 2 | 0 | 0 |
| 5 | 1492737 Flavobacterium gilvum | 2 | 2 | 0 | 0 |
| 5 | 1423322 Flavobacterium sp. ABG | 2 | 2 | 0 | 0 |
| 5 | 1341165 Flavobacterium enshiense | 2 | 2 | 0 | 0 |
| 5 | 1159016 Flavobacterium marinum | 2 | 2 | 0 | 0 |
| 5 | 503361 Flavobacterium sasangense | 2 | 2 | 0 | 0 |
| 5 | 387094 Flavobacterium hercynium | 2 | 2 | 0 | 0 |
| 5 | 370974 Flavobacterium filum | 2 | 2 | 0 | 0 |
| 5 | 350894 Flavobacterium suncheonense | 2 | 2 | 0 | 0 |
| 5 | 2026304 Flavobacterium sp. IR1 | 1 | 1 | 0 | 0 |
| 5 | 1736533 Flavobacterium sp. Root420 | 1 | 1 | 0 | 0 |
| 5 | 1729581 Flavobacterium sp. TAB 87 | 1 | 1 | 0 | 0 |
| 5 | 1566023 Flavobacterium sp. KMS | 1 | 1 | 0 | 0 |
| 5 | 1506583 Flavobacterium sp. Fl | 1 | 1 | 0 | 0 |
| 5 | 1229487 Flavobacterium sp. WG21 | 1 | 1 | 0 | 0 |
| 5 | 1189620 Flavobacterium sp. ACAM 123 | 1 | 1 | 0 | 0 |
| 5 | 935223 Flavobacterium xueshanense | 1 | 1 | 0 | 0 |
| 5 | 728056 Flavobacterium oncorhynchi | 1 | 1 | 0 | 0 |
| 5 | 604089 Flavobacterium sinopsychrotolerans | 1 | 1 | 0 | 0 |
| 5 | 554283 Flavobacterium chungangense | 1 | 1 | 0 | 0 |
| 5 | 490189 Flavobacterium caeni | 1 | 1 | 0 | 0 |
| S | 280093 Flavobacterium granuli | 1 | 1 | 0 | 0 |
| S | 271155 Flavobacterium antarcticum | 1 | 1 | 0 | 0 |
| S | 150172 Flavobacterium tegetincola | 1 | 1 | 0 | 0 |
| S | 95618 Flavobacterium sp. B17 | 1 | 1 | 0 | 0 |
| S | 29533 Flavobacterium pectinovorum | 1 | 1 | 0 | 0 |
| G | 59732 Chryseobacterium | 235 | 0 | 0 | 0 |
| S | 558151 Chryseobacterium angstadtii | 23 | 23 | 0 | 0 |
| S | 1414727 Chryseobacterium artocarpi | 23 | 23 | 0 | 0 |
| S | 1736361 Chryseobacterium sp. Leaf394 | 11 | 11 | 0 | 0 |
| S | 253 Chryseobacterium indologenes | 45 | 45 | 0 | 0 |
| S | 421531 Chryseobacterium luteum | 8 | 8 | 0 | 0 |
| S | 2015076 Chryseobacterium sp. T16E-39 | 7 | 7 | 0 | 0 |
| S | 2010998 Chryseobacterium sp. VAUSW3 | 7 | 7 | 0 | 0 |
| S | 1302685 Chryseobacterium takakiae | 6 | 6 | 0 | 0 |
| S | 1907398 Chryseobacterium sp. RU33C | 5 | 5 | 0 | 0 |
| S | 680127 Chryseobacterium culicis | 5 | 5 | 0 | 0 |
| S | 266749 Chryseobacterium jeonii | 5 | 5 | 0 | 0 |
| S | 1816690 Chryseobacterium sp. 6021061333 | 4 | 4 | 0 | 0 |
| S | 1735672 Chryseobacterium sp. Leaf201 | 4 | 4 | 0 | 0 |
| S | 1416778 Chryseobacterium arachidis | 4 | 4 | 0 | 0 |
| S | 1125876 Chryseobacterium frigidisoli | 4 | 4 | 0 | 0 |
| S | 421530 Chryseobacterium bovis | 4 | 4 | 0 | 0 |
| S | 1869230 Chryseobacterium sp. CBo1 | 3 | 3 | 0 | 0 |
| S | 1500292 Chryseobacterium sp. YR459 | 3 | 3 | 0 | 0 |
| S | 1500285 Chryseobacterium sp. OV279 | 3 | 3 | 0 | 0 |
| S | 1324352 Chryseobacterium gallinarum | 3 | 3 | 0 | 0 |
| S | 631455 Chryseobacterium treverense | 3 | 3 | 0 | 0 |
| S | 558152 Chryseobacterium piperi | 3 | 3 | 0 | 0 |
| S | 536441 Chryseobacterium taklimakanense | 3 | 3 | 0 | 0 |
| S | 454006 Chryseobacterium hungaricum | 3 | 3 | 0 | 0 |
| S | 267125 Chryseobacterium kwangjuense | 3 | 3 | 0 | 0 |
| S | 1500298 Chryseobacterium sp. YR561 | 2 | 2 | 0 | 0 |
| S | 1500294 Chryseobacterium sp. YR485 | 2 | 2 | 0 | 0 |
| S | 1500286 Chryseobacterium sp. OV259 | 2 | 2 | 0 | 0 |
| S | 1500282 Chryseobacterium sp. CF365 | 2 | 2 | 0 | 0 |
| S | 1453492 Chryseobacterium hispalense | 2 | 2 | 0 | 0 |
| S | 1416779 Chryseobacterium zeae | 2 | 2 | 0 | 0 |
| S | 1141221 Chryseobacterium taihuense | 2 | 2 | 0 | 0 |
| S | 1124835 Chryseobacterium carnipullorum | 2 | 2 | 0 | 0 |
| S | 373672 Chryseobacterium gambrini | 2 | 2 | 0 | 0 |
| S | 345663 Chryseobacterium greenlandense | 2 | 2 | 0 | 0 |
| S | 311334 Chryseobacterium taeanense | 2 | 2 | 0 | 0 |
| S | 250 Chryseobacterium gleum | 2 | 2 | 0 | 0 |
| S | 1736289 Chryseobacterium sp. Leaf180 | 1 | 1 | 0 | 0 |
| S | 1705393 Chryseobacterium sp. ERMR1: 04 | 1 | 1 | 0 | 0 |
| S | 1685010 Chryseobacterium sp. IHBB 10212 | 1 | 1 | 0 | 0 |
| S | 1302687 Chryseobacterium polytrichastri | 1 | 1 | 0 | 0 |
| S | 1233951 Chryseobacterium sp. FH1 | 1 | 1 | 0 | 0 |
| S | 1233950 Chryseobacterium sp. JM1 | 1 | 1 | 0 | 0 |
| S | 885586 Chryseobacterium sp. BLS98 | 1 | 1 | 0 | 0 |
| S | 510955 Chryseobacterium solincola | 1 | 1 | 0 | 0 |
| S | 445961 Chryseobacterium soli | 1 | 1 | 0 | 0 |
| S | 421072 Chryseobacterium halperniae | 1 | 1 | 0 | 0 |
| S | 412438 Chryseobacterium daeguense | 1 | 1 | 0 | 0 |
| S | 356305 Chryseobacterium wanjuense | 1 | 1 | 0 | 0 |
| S | 311333 Chryseobacterium soldanellicola | 1 | 1 | 0 | 0 |
| S | 307480 Chryseobacterium vrystaatense | 1 | 1 | 0 | 0 |
| S | 295069 Chryseobacterium taichungense | 1 | 1 | 0 | 0 |
| S | 232216 Chryseobacterium koreense | 1 | 1 | 0 | 0 |
| S | 112234 Chryseobacterium joostei | 1 | 1 | 0 | 0 |
| S | 254 Chryseobacterium indoltheticum | 1 | 1 | 0 | 0 |
| G | 308865 Elizabethkingia | 256 | 0 | 0 | 0 |
| S | 1756149 Elizabethkingia genomosp. 3 | 233 | 233 | 0 | 0 |
| S | 1117645 Elizabethkingia anophelis | 13 | 13 | 0 | 0 |
| S | 238 Elizabethkingia meningoseptica | 4 | 4 | 0 | 0 |
| S | 1756150 Elizabethkingia genomosp. 4 | 3 | 3 | 0 | 0 |
| S | 172045 Elizabethkingia miricola | 2 | 2 | 0 | 0 |
| G | 1016 Capnocytophaga | 104 | 0 | 0 | 0 |
| S | 1848903 Capnocytophaga canis | 53 | 53 | 0 | 0 |
| S | 712213 Capnocytophaga sp. oral taxon 332 | 13 | 13 | 0 | 0 |
| S | 706435 Capnocytophaga sp. oral taxon 329 | 6 | 6 | 0 | 0 |
| S | 710239 Capnocytophaga sp. oral taxon 338 | 5 | 5 | 0 | 0 |
| S | 1227265 Capnocytophaga sp. oral taxon 863 | 4 | 4 | 0 | 0 |
| S | 1019 Capnocytophaga sputigena | 4 | 4 | 0 | 0 |
| S | 45242 Capnocytophaga granulosa | 3 | 3 | 0 | 0 |
| S | 28189 Capnocytophaga cynodegmi | 3 | 3 | 0 | 0 |
| S | 28188 Capnocytophaga canimorsus | 3 | 3 | 0 | 0 |
| S | 1017 Capnocytophaga gingivalis | 3 | 3 | 0 | 0 |
| S | 936370 Capnocytophaga sp. CM59 | 2 | 2 | 0 | 0 |
| S | 1018 Capnocytophaga ochracea | 2 | 2 | 0 | 0 |
| S | 712215 Capnocytophaga sp. oral taxon 335 | 1 | 1 | 0 | 0 |
| S | 45243 Capnocytophaga haemolytica | 1 | 1 | 0 | 0 |
| G | 261827 Algibacter | 89 | 0 | 0 | 0 |
| S | 1849968 Algibacter aquaticus | 46 | 46 | 0 | 0 |
| S | 221126 Algibacter lectus | 42 | 42 | 0 | 0 |
| S | 870482 Algibacter pectinivorans | 1 | 1 | 0 | 0 |
| G | 49277 Psychroserpens | 59 | 0 | 0 | 0 |
| S | 943936 Psychroserpens damuponensis | 44 | 44 | 0 | 0 |
| S | 1250004 Psychroserpens sp. Hel_1_66 | 7 | 7 | 0 | 0 |
| S | 49278 Psychroserpens burtonensis | 7 | 7 | 0 | 0 |
| S | 1484460 Psychroserpens jangbogonensis | 1 | 1 | 0 | 0 |
| G | 292691 Gramella | 51 | 0 | 0 | 0 |
| S | 1913577 Gramella sp. LPB0144 | 37 | 37 | 0 | 0 |
| S | 1250205 Gramella sp. MAR_2010_147 | 8 | 8 | 0 | 0 |
| S | 279359 Gramella echinicola | 3 | 3 | 0 | 0 |
| S | 326069 Gramella portivictoriae | 2 | 2 | 0 | 0 |
| S | 1250231 Gramella sp. MAR_2010_102 | 1 | 1 | 0 | 0 |
| G | 76831 Myroides | 50 | 0 | 0 | 0 |
| S | 1150112 Myroides guanonis | 7 | 7 | 0 | 0 |
| S | 76832 Myroides odoratimimus | 3 | 3 | 0 | 0 |
| S | 256 Myroides odoratus | 1 | 1 | 0 | 0 |
| G | 290174 Aquimarina | 41 | 0 | 0 | 0 |
| S | 1038014 Aquimarina amphilecti | 17 | 17 | 0 | 0 |
| S | 987 Aquimarina latercula | 7 | 7 | 0 | 0 |
| S | 1642818 Aquimarina sp. RZW4-3-2 | 5 | 5 | 0 | 0 |
| S | 1296415 Aquimarina pacifica | 5 | 5 | 0 | 0 |
| S | 980584 Aquimarina agarivorans | 3 | 3 | 0 | 0 |
| S | 279356 Aquimarina muelleri | 2 | 2 | 0 | 0 |
| S | 1443666 Aquimarina megaterium | 1 | 1 | 0 | 0 |
| S | 666563 Aquimarina macrocephali | 1 | 1 | 0 | 0 |
| G | 52959 Polaribacter | 38 | 0 | 0 | 0 |
| S | 1888915 Polaribacter haliotis | 9 | 9 | 0 | 0 |
| S | 996801 Polaribacter reichenbachii | 7 | 7 | 0 | 0 |
| S | 1250006 Polaribactersp. Hel_1_88 | 2 | 2 | 0 | 0 |
| S | 1250005 Polaribactersp. Hel1_85 | 2 | 2 | 0 | 0 |
| S | 1336804 Polaribactersp. Hel1_33_78 | 1 | 1 | 0 | 0 |

TABLE 12
Example of a GutBuster ™ Report Detailing the Abundance
of the Top 10 Organisms Found in an Exemplary Stool Sample

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| S | 820 Bacteroides uniformis | 1398898 | 1398898 | 10.24 | 13.11 |
| S | 214856 Alistipes finegoldii | 683209 | 683209 | 5 | 6.4 |
| S | 46506 Bacteroides stercoris | 656815 | 656815 | 4.81 | 6.15 |
| S | 821 Bacteroides vuigatus | 570952 | 570952 | 4.18 | 5.35 |
| S | 287 Pseudomonas aeruginosa | 464292 | 464292 | 3.4 | 4.35 |
| S | 562 Escherichia coil | 460793 | 460793 | 3.37 | 4.32 |
| S | 28116 Bacteroides ovatus | 376631 | 376631 | 2.76 | 3.53 |
| S | 39492 [Eubacterium] siraeum | 348382 | 348382 | 2.55 | 3.26 |
| S | 817 Bacteroides fragiiis | 308172 | 308172 | 2.26 | 2.89 |
| S | 28117 Alistipes putredinis | 291675 | 291675 | 2.14 | 2.73 |

TABLE 13
Example of a GutBuster ™ Report Detailing the Abundance of
the Influencers Organisms Found in an Exemplary Stool Sample

| | | Assigned | Assigned | | Corrected |
|---|---|---|---|---|---|
| Taxonomy | | reads | reads | Relative | relative |
| levels | Taxonomy ID Organisms | (tree) | (taxon) | abundances | abundances |
| S | 328814 Alistipes shahii | 246655 | 246655 | 1.81 | 2.31 |
| S | 46503 Parabacteroides merdae | 244990 | 244990 | 1.79 | 2.3 |
| S | 853 Faecalibacterium prausnitzii | 238187 | 238187 | 1.74 | 2.23 |
| S | 1150298 Fusicatenibacter saccharivorans | 210553 | 210553 | 1.54 | 1.97 |
| S | 328813 Alistipes onderdonkii | 196619 | 196619 | 1.44 | 1.84 |
| S | 33039 [Ruminococcus] torques | 194902 | 194902 | 1.43 | 1.83 |
| S | 239935 Akkermansia muciniphila | 177149 | 177149 | 1.3 | 1.66 |
| S | 39491 [Eubacterium] rectale | 172572 | 172572 | 1.26 | 1.62 |
| S | 1581179 Clostridium sp. HMSC19A11 | 138737 | 138737 | 1.02 | 1.3 |
| S | 908612 Alistipes sp. HGBS | 119109 | 119109 | 0.87 | 1.12 |
| | 823 Parabacteroides distasonis | 113923 | 113923 | 0.83 | 1.07 |
| S | 47678 Bacteroides caccae | 107493 | 107493 | 0.79 | 1.01 |
| S | 1463165 Klebsiella quasipneumoniae | 103357 | 103357 | 0.76 | 0.97 |
| S | 410072 Coprococcus comes | 85711 | 85711 | 0.63 | 0.8 |
| S | 457394 Bacteroides sp. 4_3_47FAA | 82836 | 82836 | 0.61 | 0.78 |
| S | 1496 Clostridioides difficile | 68751 | 68751 | 0.5 | 0.64 |
| S | 28118 Odoribacter splanchnicus | 63959 | 63959 | 0.47 | 0.6 |
| S | 328812 Parabacteroides goldsteinii | 54004 | 54004 | 0.4 | 0.51 |
| S | 1499682 Alistipes sp. AL-1 | 53142 | 53142 | 0.39 | 0.5 |
| S | 418240 Blautia wexlerae | 52500 | 52500 | 0.38 | 0.49 |
| S | 649756 Anaerostipes hadrus | 48915 | 48915 | 0.36 | 0.46 |
| S | 88431 Dorea longicatena | 47143 | 47143 | 0.35 | 0.44 |
| S | 1118061 Alistipes obesi | 45000 | 45000 | 0.33 | 0.42 |
| S | 469591 Parabacteroides sp. 20_3 | 44426 | 44426 | 0.33 | 0.42 |
| S | 35833 Bilophila wadsworthia | 44069 | 44069 | 0.32 | 0.41 |
| S | 585543 Bacteroides sp. D20 | 43700 | 43700 | 0.32 | 0.41 |
| S | 1715163 Pseudomonas sp. HMSC058A10 | 42905 | 42905 | 0.31 | 0.4 |
| | 818 Bacteroides thetaiotaomicron | 42205 | 42205 | 0.31 | 0.4 |
| S | 1504823 bacterium LF-3 | 41024 | 41024 | 0.3 | 0.38 |
| S | 658662 Parabacteroides sp. D26 | 40182 | 40182 | 0.29 | 0.38 |
| S | 166486 Roseburia intestinalis | 37299 | 37299 | 0.27 | 0.35 |
| S | 556258 Bacteroides sp. D1 | 37178 | 37178 | 0.27 | 0.35 |
| | 573 Klebsiella pneumoniae | 35661 | 35661 | 0.26 | 0.33 |
| S | 29348 [Clostridium] spiroforme | 33592 | 33592 | 0.25 | 0.31 |
| S | 1470347 Alistipes ihumii | 32731 | 32731 | 0.24 | 0.31 |
| S | 469593 Bacteroides sp. 3_1_40A | 32544 | 32544 | 0.24 | 0.3 |
| S | 39488 [Eubacterium] hallii | 32172 | 32172 | 0.24 | 0.3 |
| S | 40520 Blautia obeum | 31062 | 31062 | 0.23 | 0.29 |
| S | 39486 Dorea formicigenerans | 30806 | 30806 | 0.23 | 0.29 |
| S | 169435 Anaerotruncus colihominis | 30706 | 30706 | 0.22 | 0.29 |
| S | 665949 Tannerella sp. 6_1_58FAA_CT1 | 29336 | 29336 | 0.21 | 0.27 |
| S | 1297617 Intestinimonas butyriciproducens | 28667 | 28667 | 0.21 | 0.27 |
| S | 100886 Catenibacterium mitsuokai | 28587 | 28587 | 0.21 | 0.27 |
| S | 291645 Bacteroides nordii | 28552 | 28552 | 0.21 | 0.27 |
| S | 745368 Gemmiger formicilis | 27814 | 27814 | 0.2 | 0.26 |
| S | 357276 Bacteroides dorei | 26184 | 26184 | 0.19 | 0.25 |
| S | 39485 [Eubacterium] eligens | 25952 | 25952 | 0.19 | 0.24 |
| S | 360807 Roseburia inulinivorans | 24453 | 24453 | 0.18 | 0.23 |
| S | 1099853 Coprobacter fastidiosus | 23908 | 23908 | 0.18 | 0.22 |
| S | 1531 [Clostridium] clostridioforme | 23228 | 23228 | 0.17 | 0.22 |
| S | 292800 Flavonifractor plautii | 23050 | 23050 | 0.17 | 0.22 |
| S | 74426 Collinsella aerofaciens | 21999 | 21999 | 0.16 | 0.21 |
| S | 301302 Roseburia faecis | 21335 | 21335 | 0.16 | 0.2 |
| S | 457393 Bacteroides sp. 4L1_36 | 19511 | 19511 | 0.14 | 0.18 |
| S | 592978 Ruminococcus faecis | 19271 | 19271 | 0.14 | 0.18 |
| S | 469592 Bacteroides sp. 3_1_19 | 19051 | 19051 | 0.14 | 0.18 |
| S | 1535 [Clostridium] leptum | 17531 | 17531 | 0.13 | 0.16 |
| 5 | 665956 Subdoligranulum sp. 4_3_54A2FAA | 17450 | 17450 | 0.13 | 0.16 |
| 5 | 310298 Bacteroides coprocola | 16870 | 16870 | 0.12 | 0.16 |
| 5 | 1737424 Blautia massiliensis | 16078 | 16078 | 0.12 | 0.15 |
| 5 | 1432052 Eisenbergiella tayi | 15741 | 15741 | 0.12 | 0.15 |
| 5 | 469590 Bacteroides sp. 2_2_4 | 15107 | 15107 | 0.11 | 0.14 |
| 5 | 1288121 Alistipes senegalensis | 14730 | 14730 | 0.11 | 0.14 |
| 5 | 487175 Parasutterella excrementihominis | 14498 | 14498 | 0.11 | 0.14 |
| 5 | 571438 Desulfovibrio legallii | 13019 | 13019 | 0.1 | 0.12 |
| 5 | 469610 Burkholderiales bacterium 1_1_47 | 12688 | 12688 | 0.09 | 0.12 |
| 5 | 1519439 Oscillibacter sp. ER4 | 12032 | 12032 | 0.09 | 0.11 |
| 5 | 1661050 Pseudomonas sp. NBRC 111135 | 11809 | 11809 | 0.09 | 0.11 |
| 5 | 33038 [Ruminococcus] gnavus | 11385 | 11385 | 0.08 | 0.11 |
| 5 | 1550024 Ruthenibacterium lactatiformans | 11146 | 11146 | 0.08 | 0.1 |
| 5 | 371601 Bacteroides xylanisolvens | 10839 | 10839 | 0.08 | 0.1 |
| S | 1520805 Blautia sp. 5F-50 | 10647 | 10647 | 0.08 | 0.1 |
| 5 | 1882754 Clostridium sp. DSM 4029 | 10556 | 10556 | 0.08 | 0.1 |
| 5 | 2025876 Parabacteroides sp. CTO6 | 10490 | 10490 | 0.08 | 0.1 |

TABLE 14
Report of the Abundance of Gram-positive Bacteria
Found in an Exemplary Stool Sample

| | Abundance | Reads |
|---|---|---|
| Bifidobacterium coagilans | #N/A | #N/A |
| Bifidobacterium infantis | #N/A | #N/A |
| Bifidobacterium lactis | #N/A | #N/A |
| Bifidobacterium subtilis | #N/A | #N/A |
| Lactobacillus bulgaricus | #N/A | #N/A |
| Lactobacillus lactis | #N/A | #N/A |
| Saccharomyces boulardii | #N/A | #N/A |
| Lactobacillus helveticus | #N/A | #N/A |
| Enterococcus faecium | 0.05 | 5235 |
| Streptococcus thermophilus | 0.03 | 3233 |
| Bifidobacterium longum | 0.01 | 908 |
| Bifidobacterium bifidum | 0 | 127 |
| Lactobacillus salivarius | 0 | 123 |
| Lactobacillus paracasei | 0 | 83 |
| Bifidobacterium breve | 0 | 77 |
| Bacillus subtilis | 0 | 71 |
| Bacillus coagulans | 0 | 69 |
| Lactobacillus plantarum | 0 | 68 |
| Lactobacillus delbrueckii | 0 | 58 |
| Bifidobacterium animalis | 0 | 51 |
| Lactobacillus casei | 0 | 33 |
| Lactobacillus acidophilus | 0 | 24 |
| Lactobacillus gasseri | 0 | 22 |
| Lactobacillus reuteri | 0 | 18 |
| Lactobacillus rhamnosus | 0 | 3 |

TABLE 15
Listing of Organisms Found in the Gut Microbiome

| | | | | |
|---|---|---|---|---|
| [Actinopolyspora] | [Bacillus] | [Bacillus] | [Bacillus] | [Bacteroides] |
| iraqiensis | clarkii | selenitireducens | sp. KCTC 13219 | pectinophilus |
| [Brevibacterium] | [Butyribacterium] | [Candida] | [Candida] | [Candida] |
| frigoritolerans | methylotrophicum | auris | glabrata | tanzawaensis |
| [Candida] | [Clostridium] | [Clostridium] | [Clostridium] | [Clostridium] |
| tenuis | aerotolerans | aminophilum | asparagiforme | bolteae |
| [Clostridium] | [Clostridium] | [Clostridium] | [Clostridium] | [Clostridium] |
| celerecrescens | cellobioparum | cellulolyticum | cellulosi | citroniae |
| [Clostridium] | [Clostridium] | [Clostridium] | [Clostridium] | [Clostridium] |
| clariflavum | clostridioforme | cocleatum | dakarense | fimetarium |
| [Clostridium] | [Clostridium] | [Clostridium] | [Clostridium] | [Clostridium] |
| glycyrrhizinilyticum | hiranonis | hungatei | hylemonae | indolis |
| [Clostridium] | [Clostridium] | [Clostridium] | [Clostridium] | [Clostridium] |
| innocuum | josui | lactatifermentans | lavalense | leptum |
| [Clostridium] | [Clostridium] | [Clostridium] | [Clostridium] | [Clostridium] |
| methoxybenzovorans | methylpentosum | neopropionicum | papyrosolvens | paradoxum |
| [Clostridium] | [Clostridium] | [Clostridium] | [Clostridium] | [Clostridium] |
| polysaccharolyticum | populeti | propionicum | purinilyticum | saccharogumia |
| [Clostridium] | [Clostridium] | [Clostridium] | [Clostridium] | [Clostridium] |
| saccharolyticum | scindens | spiroforme | sporosphaeroides | stercorarium |
| [Clostridium] | [Clostridium] | [Clostridium] | [Clostridium] | [Clostridium] |
| straminisolvens | symbiosum | termitidis | thermoalcaliphilum | ultunense |
| [Clostridium] | [Collinsella] | [Desiilfotomaculum] | [Eubacterium] | [Eubacterium] |
| viride | massiliensis | guttoideum | angustum | brachy |
| [Eubacterium] | [Eubacterium] | [Eubacterium] | [Eubacterium] | [Eubacterium] |
| cellulosolvens | dolichum | eligens | hallii | infirmum |
| [Eubacterium] | [Eubacterium] | [Eubacterium] | [Eubacterium] | [Eubacterium] |
| nodatum | rectale | saphenum | siraeum | sulci |
| [Eubacterium] | [Flexibacter] | [Flexibacter] | [Haemophilus] | [Haemophilus] |
| yurii | sp. ATCC 35103 | sp. ATCC 35208 | ducreyi | felis |
| [Haemophilus] | [Hallella] | [Mannheimia] | [Polyangium] | [Propionibacterium] |
| parasuis | seregens | succiniciproducens | brachysporum | namnetense |
| [Pseudomonas] | [Pseudomonas] | [Pseudomonas] | [Ruminococcus] | [Ruminococcus] |
| cissicola | geniculata | mesoacidophila | gnavus | torques |
| [Scytonema | Abiotrophia | Abiotrophia | Acanthamoeba | Acanthamoeba |
| hofmanni] | defectiva | sp. HMSC24B09 | castellanii | polyphaga mimivirus |
| UTEX B 1581 | | | | |
| Acaricomes | Acaryochloris | Acaryochloris | Acetanaerobacterium | Acetitomaculum |
| phytoseiuli | marina | sp. CCMEE 5410 | elongatum | ruminis |
| Acetivibrio | Acetivibrio | Acetoanaerobium | Acetoanaerobium | Acetobacter |
| cellulolyticus | ethanolgignens | noterae | sticklandii | aceti |
| Acetobacter | Acetobacter | Acetobacter | Acetobacter | Acetobacter |
| cerevisiae | cibinongensis | fabarum | ghanensis | indonesiensis |
| Acetobacter | Acetobacter | Acetobacter | Acetobacter | Acetobacter |
| malorum | nitrogenifigens | okinawensis | orientalis | orleanensis |
| Acetobacter | Acetobacter | Acetobacter | Acetobacter | Acetobacter |
| papayae | pasteurianus | persici | senegalensis | sp. DmW_043 |
| Acetobacter | Acetobacter | Acetobacter | Acetobacter | Acetobacter |
| sp. DsW_059 | sp. DsW_063 | sp. DsW_47 | sp. DsW_54 | sp. SLV-7 |
| Acetobacter | Acetobacter | Acetobacteraceae | Acetobacterium | Acetobacterium |
| syzygii | tropicalis | bacterium | bakii | dehalogenans |
| | | AT-5844 | | |
| Acetobacterium | Acetobacterium | Acetohalobium | Acetomicrobium | Acetomicrobium |
| wieringae | woodii | arabaticum | flavidum | hydrogeniformans |
| Acetomicrobium | Acetomicrobium | Acetonema | Acholeplasma | Acholeplasma |
| mobile | thermoterrenum | longum | axanthum | brassicae |
| Acholeplasma | Acholeplasma | Acholeplasma | Acholeplasma | Acholeplasma |
| equifetale | granularum | hippikon | laidlawii | modicum |
| Acholeplasma | Acholeplasma | Acholeplasma | Achromobacter | Achromobacter |
| multilocale | oculi | palmae | arsenitoxydans | denitrificans |
| Achromobacter | Achromobacter | Achromobacter | Achromobacter | Achromobacter |
| insolitus | insuavis | marplatensis | piechaudii | ruhlandii |
| Achromobacter | Achromobacter | Achromobacter | Achromobacter | Achromobacter |
| sp. 2789STDY5608606 | sp. 2789STDY5608615 | sp. 2789STDY5608618 | sp. 2789STDY5608619 | sp. 2789STDY5608621 |
| Achromobacter | Achromobacter | Achromobacter | Achromobacter | Achromobacter |
| sp. 2789STDY5608622 | sp. 2789STDY5608623 | sp. 2789STDY5608625 | sp. 2789STDY5608629 | sp. 2789STDY5663429 |
| Achromobacter | Achromobacter | Achromobacter | Achromobacter | Achromobacter |
| F sp. AR476-2 | sp. ATCC31444 | sp. DH1f | sp. DMS1 | sp. HMSC056C09 |
| Achromobacter | Achromobacter | Achromobacter | Achromobacter | Achromobacter |
| sp. HMSC15D03 | sp. LC458 | sp. MFA1 R4 | sp. NFACC18-2 | sp. Root170 |
| Achromobacter | Achromobacter | Achromobacter | Achromobacter | Achromobacter |
| sp. Root565 | sp. Root83 | sp. RTa | spanius | xylosoxidans |
| Acidaminobacter | Acidaminococcus | Acidaminococcus | Acidaminococcus | Acidaminococcus |
| hydrogenoformans | fermentans | intestini | massiliensis | sp. BV3L6 |
| Acidaminococcus | Acidaminococcus | Acidaminococcus | Acidianus | Acidibacillus |
| sp. D21 | sp. HPA0509 | timonensis | hospitalis | ferrooxidans |
| Acidiferrobacter | Acidihalobacter | Acidihalobacter | Acidimicrobium | Acidiphilium |
| thiooxydans | ferrooxidans | prosperus | ferrooxidans | cryptum |
| Acidiphilium | Acidiphilium | Acidiphilium | Acidiplasma | Acidipropionibacterium |
| multivorum | sp. JA12-A1 | sp. PM | ciipricumulans | acidipropionici |
| Acidipropionibacterium | Acidipropionibacterium | Acidisphaera | Acidithiobacillus | Acidithiobacillus |
| jensenii | thoenii | rubrifaciens | caldus | ferrivorans |
| Acidithiobacillus | Acidithiobacillus | Acidithrix | Acidobacteria | Acidobacteriaceae |
| ferrooxidans | thiooxidans | ferrooxidans | bacterium KBS 146 | bacterium KBS 83 |
| Acidobacteriaceae | Acidobacteriaceae | Acidobacteriaceae | Acidobacteriaceae | Acidobacterium |
| bacterium KBS 89 | bacterium KBS 96 | bacterium TAA166 | bacterium URHE0068 | ailaaui |
| Acidobacterium | Acidocella | Acidocella | Acidocella | Acidomonas |
| capsulatum | aminolytica | facilis | sp. MX-AZ02 | methanolica |
| Acidothermus | Acidovorax | Acidovorax | Acidovorax | Acidovorax |
| cellulolyticus | avenae | caeni | cattleyae | citrulli |
| Acidovorax | Acidovorax | Acidovorax | Acidovorax | Acidovorax |
| delafieldii | ebreus | konjaci | oryzae | radicis |
| Acidovorax | Acidovorax | Acidovorax | Acidovorax | Acidovorax |
| soli | sp. 12322-1 | sp. CCH12-A4 | sp. CF316 | sp. GW101-3H11 |
| Acidovorax | Acidovorax | Acidovorax | Acidovorax | Acidovorax |
| sp. JHL-3 | sp. JHL-9 | sp. JS42 | sp. KKS102 | sp. KNDSW-TSA6 |
| Acidovorax | Acidovorax | Acidovorax | Acidovorax | Acidovorax |
| sp. Leaf160 | sp. Leaf78 | sp. MR-S7 | sp. NA2 | sp. NA3 |
| Acidovorax | Acidovorax | Acidovorax | Acidovorax | Acidovorax |
| sp. NO-1 | sp. RAC01 | sp. Root217 | sp. Root219 | sp. Root267 |
| Acidovorax | Acidovorax | Acidovorax | Acidovorax | Acidovorax |
| sp. Root275 | sp. Root402 | sp. Root568 | sp. Root70 | sp. SD340 |
| Acidovorax | Acidovorax | Acidovorax | Aciduliprofundum | Aciduliprofundum |
| temperans | valerianellae | wautersii | boonei | sp. MAR08-339 |
| Acinetobacter | Acinetobacter | Acinetobacter | Acinetobacter | Acinetobacter |
| albensis | apis | baumannii | beijerinckii | bereziniae |
| Acinetobacter | Acinetobacter | Acinetobacter | Acinetobacter | Acinetobacter |
| bohemicus | boissieri | bouvetii | brisouii | calcoaceticus |
| Acinetobacter | Acinetobacter | Acinetobacter | Acinetobacter | Acinetobacter |
| celticus | defluvii | dijkshoorniae | equi | gandensis |
| Acinetobacter | Acinetobacter | Acinetobacter | Acinetobacter | Acinetobacter |
| genomosp. 33YU | gerneri | guillouiae | gyllenbergii | haemolyticus |
| Acinetobacter | Acinetobacter | Acinetobacter | Acinetobacter | Acinetobacter |
| harbinensis | idrijaensis | indicus | johnsonii | junii |
| Acinetobacter | Acinetobacter | Acinetobacter | Acinetobacter | Acinetobacter |
| kookii | larvae | lwoffii | marinus | nectaris |
| Acinetobacter | Acinetobacter | Acinetobacter phage | Acinetobacter | Acinetobacter |
| nosocomialis | parvus | LZ35 | pittii | populi |
| Acinetobacter | Acinetobacter | Acinetobacter | Acinetobacter | Acinetobacter |
| proteolyticus | qingfengensis | radioresistens | schindleri | soli |
| Acinetobacter | Acinetobacter | Acinetobacter | Acinetobacter | Acinetobacter |
| sp. 1294596 | sp. 25977_10 | sp. A47 | sp. Ag2 | sp. ANC 3789 |
| Acinetobacter | Acinetobacter | Acinetobacter | Acinetobacter | Acinetobacter |
| sp. ANC 3813 | sp. ANC 3832 | sp. ANC 3862 | sp. ANC 3880 | sp. ANC 3903 |
| Acinetobacter | Acinetobacter | Acinetobacter | Acinetobacter | Acinetobacter |
| sp. ANC 3929 | sp. ANC 4105 | sp. ANC 4149 | sp. ANC 4169 | sp. ANC 4204 |
| Acinetobacter | Acinetobacter | Acinetobacter | Acinetobacter | Acinetobacter |
| sp. ANC 4218 | sp. ANC 4470 | sp. ANC 4648 | sp. ANC 4654 | sp. ANC 4655 |
| Acinetobacter | Acinetobacter | Acinetobacter | Acinetobacter | Acinetobacter |
| sp. ANC 4945 | sp. ANC 4973 | sp. ANC 4999 | sp. ANC 5054 | sp. ANC 5600 |
| Acinetobacter | Acinetobacter | Acinetobacter | Acinetobacter | Acinetobacter |
| sp. CIP 102129 | sp. CIP 102136 | sp. CIP 110321 | sp. CIP 51.11 | sp. CIP 56.2 |
| Acinetobacter | Acinetobacter | Acinetobacter | Acinetobacter | Acinetobacter |
| sp. CIP 64.2 | sp. CIP-A165 | sp. COS3 | sp. DSM 11652 | sp. ETR1 |
| Acinetobacter | Acinetobacter | Acinetobacter | Acinetobacter | Acinetobacter |
| sp. GG2 | sp. HR7 | sp. LCT-H3 | sp. LW15 | sp. MDS7A |
| Acinetobacter | Acinetobacter | Acinetobacter | Acinetobacter | Acinetobacter |
| sp. MF4640 | sp. MF4642 | sp. MN12 | sp. NBRC 110496 | sp. NCu2D-2 |
| Acinetobacter | Acinetobacter | Acinetobacter | Acinetobacter | Acinetobacter |
| sp. neg1 | sp. NIPH 1847 | sp. NIPH 2036 | sp. NIPH 2168 | sp. NIPH 2171 |
| Acinetobacter | Acinetobacter | Acinetobacter | Acinetobacter | Acinetobacter |
| sp. NIPH 236 | sp. NIPH 284 | sp. NIPH 3623 | sp. NIPH 542 | sp. NIPH 809 |
| Acinetobacter | Acinetobacter | Acinetobacter | Acinetobacter | Acinetobacter |
| sp. NIPH 817 | sp. NRRL B-65365 | sp. P8-3-8 | sp. Root1280 | sp. SFA |
| Acinetobacter | Acinetobacter | Acinetobacter | Acinetobacter | Acinetobacter |
| sp. SFB | sp. SFC | sp. SFD | sp. TGL-Y2 | sp. Ver3 |
| Acinetobacter | Acinetobacter | Acinetobacter | Acinetobacter | Acinetobacter |
| sp. WC-323 | sp. WC-743 | sp. WCHA29 | sp. WCHA34 | sp. WCHA45 |
| Acinetobacter | Acinetobacter | Acinetobacter | Acinetobacter | Acinetobacter |
| sp. WCHA5 | sp. WCHA52 | sp. WCHA55 | sp. WCHA60 | sp. WCHA62 |
| Acinetobacter | Acinetobacter | Acinetobacter | Acinetobacter | Acinetobacter |
| sp. YK3 | sp. yr461 | sp. YZS-X1-1 | tandoii | towneri |
| Acinetobacter | Acinetobacter | Actibacterium | Actibacterium | Actibacterium |
| ursingii | venetianus | atlanticum | mucosum | sp. JN33 |
| Actibacterium | Actinoalloteichus | Actinoalloteichus | Actinoalloteichus | Actinoalloteichus |
| ureilyticum | cyanogriseus | hoggarensis | hymeniacidonis | sp. ADI127-7 |
| Actinoalloteichus | Actinobacillus | Actinobacillus | Actinobacillus | Actinobacillus |
| spitiensis | capsulatus | equuli | minor | pleuropneumoniae |
| Actinobacillus | Actinobacillus | Actinobacillus | Actinobacillus | Actinobacteria |
| seminis | succinogenes | suis | ureae | bacterium |
| | | | | DSM 45722 |
| Actinobacteria | Actinobacteria | Actinobacteria | Actinobacteria | Actinobacteria |
| bacterium | bacterium | bacterium | bacterium | bacterium |
| IMCC25003 | IMCC26077 | IMCC26103 | IMCC26207 | IMCC26256 |
| Actinobacteria | Actinobacteria | Actinobacteria | Actinobacteria | Actinobacteria |
| bacterium OK006 | bacterium | bacterium OV320 | bacterium OV450 | bacterium |
| | OK074 | | | UC5.1-1B11 |
| actinobacterium | Actinobaculum | Actinobaculum | Actinobaculum | Actinocatenispora |
| LLX17 | massiliense | sp. oral | suis | sera |
| | | taxon 183 | | |
| Actinokineospora | Actinokineospora | Actinokineospora | Actinokineospora | Actinokineospora |
| bangkokensis | enzanensis | inagensis | spheciospongiae | terrae |
| Actinomadura | Actinomadura | Actinomadura | Actinomadura | Actinomadura |
| atramentaria | chibensis | flavalba | formosensis | hibisca |
| Actinomadura | Actinomadura | Actinomadura | Actinomadura | Actinomadura |
| kijaniata | latina | macra | madurae | mexicana |
| Actinomadura | Actinomadura | Actinomadura | Actinomadura | Actinomadura |
| meyerae | oligospora | rifamycini | rubrobrunea | sp. CNU-125 |
| Actinomyces | Actinomyces | Actinomyces | Actinomyces | Actinomyces |
| cardiffensis | coleocanis | dentalis | denticolens | europaeus |
| Actinomyces | Actinomyces | Actinomyces | Actinomyces | Actinomyces |
| gaoshouyii | georgiae | gerencseriae | glycerinitolerans | graevenitzii |
| Actinomyces | Actinomyces | Actinomyces | Actinomyces | Actinomyces |
| hordeovulneris | ihumii | israelii | johnsonii | liubingyangii |
| Actinomyces | Actinomyces | Actinomyces | Actinomyces | Actinomyces |
| marimammalium | massiliensis | meyeri | naeslundii | nasicola |
| Actinomyces | Actinomyces | Actinomyces | Actinomyces | Actinomyces |
| odontolyticus | oris | polynesiensis | provencensis | radicidentis |
| Actinomyces | Actinomyces | Actinomyces | Actinomyces | Actinomyces |
| radingae | ruminicola | slackii | sp. CtC 72 | sp. HMSC035G02 |
| Actinomyces | Actinomyces | Actinomyces | Actinomyces | Actinomyces |
| sp. HMSC062G12 | sp. HMSC065F11 | sp. HMSC065F12 | sp. HMSC075B09 | sp. HMSC075C01 |
| Actinomyces | Actinomyces | Actinomyces | Actinomyces | Actinomyces |
| sp. HMSC08A09 | sp. HPA0247 | sp. ICM39 | sp. 1CM47 | sp. 1CM54 |
| Actinomyces | Actinomyces | Actinomyces | Actinomyces | Actinomyces |
| sp. ICM58 | sp. Marseille-P2825 | sp. Marseille-P3257 | sp. oral | sp. oral |
| | | | taxon 170 | taxon 171 |
| Actinomyces | Actinomyces | Actinomyces | Actinomyces | Actinomyces |
| sp. oral | sp. oral | sp. oral | sp. oral | sp. oral |
| taxon 172 | taxon 175 | taxon 178 | taxon 180 | taxon 181 |
| Actinomyces | Actinomyces | Actinomyces | Actinomyces | Actinomyces |
| sp. oral | sp. oral | sp. oral | sp. oral | sp. oral |
| taxon 414 | taxon 448 | taxon 848 | taxon 849 | taxon 877 |
| Actinomyces | Actinomyces | Actinomyces | Actinomyces | Actinomyces |
| sp. ph3 | sp. S4-C9 | sp. S6-Spd3 | sp. VUL4_3 | suimastitidis |
| Actinomyces | Actinomyces | Actinomyces | Actinomyces | Actinomyces |
| timonensis | turicensis | urinae | urogenitalis | vaccimaxillae |
| Actinomyces | Actinomyces | Actinomyces | Actinomycetaceae | Actinomycetales |
| virus Av1 | viscosus | vulturis | bacterium BA112 | bacterium JB111 |
| Actinomycetospora | Actinophytocola | Actinophytocola | Actinoplanes | Actinoplanes |
| chiangmaiensis | xanthii | xinjiangensis | awajinensis | derwentensis |
| Actinoplanes | Actinoplanes | Actinoplanes | Actinoplanes | Actinoplanes |
| friuliensis | globispoms | missouriensis | philippinensis | rectilineatus |
| Actinoplanes | Actinoplanes | Actinoplanes | Actinoplanes | Actinoplanes |
| regularis | sp. N902-109 | sp. SE50/110 | sp. TFC3 | subtropicus |
| Actinoplanes | Actinopolymorpha | Actinopolymorpha | Actinopolymorpha | Actinopolyspora |
| utahensis | alba | cephalotaxi | singaporensis | alba |
| Actinopolyspora | Actinopolyspora | Actinopolyspora | Actinopolyspora | Actinopolyspora |
| erythraea | halophila | mortivallis | mzabensis | righensis |
| Actinopolyspora | Actinopolyspora | Actinospica | Actinospica | Actinosporangium |
| saharensis | xinjiangensis | acidiphila | robiniae | sp. NRRL B-3428 |
| Actinosynnema | Actinosynnema | Actinotalea | Actinotignum | Actinotignum |
| mirum | sp. ALI-1.44 | ferrariae | schaalii | timonense |
| Actinotignum | Acuticoccus | Acytostelium | Adhaeribacter | Adlercreutzia |
| urinate | yangtzensis | subglobosum | aquaticus | equolifaciens |
| Advenella | Advenella | Aequorivita | Aequorivita | Aequorivita |
| kashmirensis | mimigardefordensis | capsosiphonis | soesokkakensis | sublithincola |
| Aequorivita | Aequorivita | Aeribacillus | Aeriscardovia | Aerococcus |
| viscosa | vladivostokensis | pallidus | aeriphila | christensenii |
| Aerococcus | Aerococcus | Aerococcus | Aerococcus | Aerococcus |
| sanguinicola | sp. 1KP-2016 | sp. HMSC062B07 | sp. HMSC10H05 | sp. HMSC23C02 |
| Aerococcus | Aerococcus | Aerococcus | Aerococcus | Aerococcus |
| suis | urinae | urinaeequi | urinaehominis | viridans |
| Aeromicrobium | Aeromicrobium | Aeromicrobium | Aeromicrobium | Aeromicrobium |
| choanae | erythreum | marinum | massiliense | sp. Leaf272 |
| Aeromicrobium | Aeromicrobium | Aeromicrobium | Aeromicrobium | Aeromicrobium |
| sp. PE09-221 | sp. Root236 | sp. Root344 | sp. Root472D3 | sp. Root495 |
| Aeromonas | Aeromonas | Aeromonas | Aeromonas | Aeromonas |
| allosaccharophila | aquatica | australiensis | bestiarum | bivalvium |
| Aeromonas | Aeromonas | Aeromonas | Aeromonas | Aeromonas |
| caviae | dhakensis | diversa | encheleia | enteropelogenes |
| Aeromonas | Aeromonas | Aeromonas | Aeromonas | Aeromonas |
| eucrenophila | finlandiensis | fluvialis | hydrophila | jandaei |
| Aeromonas | Aeromonas | Aeromonas | Aeromonas | Aeromonas |
| lacus | media | molluscorum | piscicola | popoffii |
| Aeromonas | Aeromonas | Aeromonas | Aeromonas | Aeromonas |
| rivuli | salmonicida | sanarellii | schubertii | simiae |
| Aeromonas | Aeromonas | Aeromonas | Aeromonas | Aeromonas |
| sobria | sp. 159 | sp. A35_P | sp. DNP9 | sp. EERV15 |
| Aeromonas | Aeromonas | Aeromonas | Aeromonas | Aeromonas |
| sp. L_1B5_3 | sp. O23A | sp. RU39B | sp. SCS5 | sp. YN13HZO-058 |
| Aeromonas | Aeromonas | Aeromonas | Aeromonas | Aeromonas |
| sp. ZOR0001 | sp. ZOR0002 | taiwanensis | tecta | veronii |
| Aeromonas | Aestuariibacter | Aestuariibacter | Aestuariimicrobium | Aestuariivita |
| virus 65 | aggregatus | salexigens | kwangyangense | atlantica |
| Aestuariivita | Afifella | Afifella | Afipia | Afipia |
| boseongensis | marina | pfennigii | broomeae | clevelandensis |
| Afipia | Afipia | Afipia | Afipia | Afipia |
| felis | massiliensis | sp. 1NLS2 | sp. GAS231 | sp. P52-10 |
| Afipia | Agaricus | Agarivorans | Agarivorans | Aggregatibacter |
| sp. Root123D2 | bisporus | albus | gilvus | actinomycetemcomitans |
| Aggregatibacter | Aggregatibacter | Aggregatibacter | Agreia | Agreia |
| aphrophilus | segnis | sp. oral taxon 458 | bicolorata | pratensis |
| Agreia | Agreia | Agreia | Agreia | Agreia |
| sp. Leaf210 | sp. Leaf244 | sp. Leaf283 | sp. Leaf335 | sp. VKM Ac-1783 |
| Agreia | Agrobacterium | Agrobacterium | Agrobacterium | Agrobacterium |
| sp. VKM Ac- 2052 | albertimagni | arsenijevicii | genomosp. 1 | genomosp. 13 |
| Agrobacterium | Agrobacterium | Agrobacterium | Agrobacterium | Agrobacterium |
| genomosp. 2 | genomosp. 3 | genomosp. 5 | genomosp. 6 | genomosp. 7 |
| Agrobacterium | Agrobacterium | Agrobacterium | Agrobacterium | Agrobacterium |
| genomosp. 9 | larrymoorei | rhizogenes | rubi | salinitolerans |
| Agrobacterium | Agrobacterium | Agrobacterium | Agrobacterium | Agrobacterium |
| sp. 10MFCol1.1 | sp. 13-2099-1-2 | sp. ATCC 31749 | sp. B133/95 | sp. D14 |
| Agrobacterium | Agrobacterium | Agrobacterium | Agrobacterium | Agrobacterium |
| sp. DSM 25558 | sp. DSM 25559 | sp. H13-3 | sp. R89-1 | sp. RAC06 |
| Agrobacterium | Agrobacterium | Agrobacterium | Agrobacterium | Agrococcus |
| sp. SUL3 | sp. YIC 4121 | tumefaciens | vitis | baldri |
| Agrococcus | Agrococcus | Agrococcus | Agrococcus | Agrococcus |
| carbonis | casei | jejuensis | lahaulensis | pavilionensis |
| Agrococcus | Agromyces | Agromyces | Agromyces | Agromyces |
| sp. Marseille-P2731 | aureus | cerinus | flavus | italicus |
| Agromyces | Agromyces | Agromyces | Agromyces | Agromyces |
| sp. CF514 | sp. Leaf222 | sp. NDB4Y10 | sp. Root1464 | sp. Root81 |
| Agromyces | Agromyces | Ahrensia | Ahrensia | Ahrensia |
| sp. Soil535 | subbeticus | marina | sp. 13_GOM-1096m | sp. R2A130 |
| Aidingimonas | Akkermansia | Akkermansia | Akkermansia | Akkermansia |
| halophila | glycaniphila | muciniphila | sp. KLE1605 | sp. KLE1797 |
| Akkermansia | Albidiferax | Albidovulum | Albimonas | Albimonas |
| sp. KLE1798 | sp. OV413 | xiamenense | donghaensis | pacifica |
| Alcaligenes | Alcaligenes | Alcanivorax | Alcanivorax | Alcanivorax |
| faecalis | sp. EGD-AK7 | borkumensis | dieselolei | hongdengensis |
| Alcanivorax | Alcanivorax | Alcanivorax | Alcanivorax | Alcanivorax |
| jadensis | nanhaiticus | pacificus | sp. 97CO-5 | sp. DG881 |
| Alcanivorax | Alcanivorax | Alcanivorax | Alcanivorax | Alcanivorax |
| sp. KX64203 | sp. N3-7A | sp. NBRC 102024 | sp. P2S70 | xenomutans |
| Algibacter | Algibacter | Algibacter | Algibacter | Algicola |
| alginicilyticus | aquaticus | lectus | pectinivorans | sagamiensis |
| Algiphilus | Algoriella | Algoriphagus | Algoriphagus | Algoriphagus |
| aromaticivorans | xinjiangensis | alkaliphilus | antarcticus | aquimarinus |
| Algoriphagus | Algoriphagus | Algoriphagus | Algoriphagus | Algoriphagus |
| faecimaris | halophilus | hitonicola | locisalis | machipongonensis |
| Algoriphagus | Algoriphagus | Algoriphagus | Algoriphagus | Algoriphagus |
| mannitolivorans | marincola | marinus | ornithinivorans | ratkowskyi |
| Algoriphagus | Algoriphagus | Algoriphagus | Algoriphagus | Algoriphagus |
| resistens | sp. A40 | sp. M8-2 | terrigena | vanfongensis |
| Algoriphagus | Aliagarivorans | Aliagarivorans | Alicycliphilus | Alicycliphilus |
| zhangzhouensis | marinus | taiwanensis | denitrificans | sp. B1 |
| Alicycliphilus | Alicycliphilus | Alicyclobacillus | Alicyclobacillus | Alicyclobacillus |
| sp. BQ1 | sp. EPL6 | acidiphilus | acidocaldarius | acidoterrestris |
| Alicyclobacillus | Alicyclobacillus | Alicyclobacillus | Alicyclobacillus | Alicyclobacillus |
| contaminans | ferrooxydans | herbarius | hesperidum | kakegawensis |
| Alicyclobacillus | Alicyclobacillus | Alicyclobacillus | Alicyclobacillus | Alicyclobacillus |
| macrosporangiidus | mali | pomorum | sendaiensis | shizuokensis |
| Alicyclobacillus | Alicyclobacillus | Alicyclobacillus | Aliicoccus | Aliifodinibius |
| sp. USBA-503 | tengchongensis | vulcanalis | persicus | roseus |
| Aliiglaciecola | Aliihoeflea | Aliiroseovarius | Aliiroseovarius | Aliivibrio |
| lipolytica | sp. 2WW | crassostreae | sediminilitoris | fischeri |
| Aliivibrio | Aliivibrio | Alishewanella | Alishewanella | Alishewanella |
| salmonicida | wodanis | aestuarii | agri | jeotgali |
| Alishewanella | Alishewanella | Alistipes | Alistipes | Alistipes |
| sp. HH-ZS | sp. WH16-1 | finegoldii | ihumii | indistinctus |
| Alistipes | Alistipes | Alistipes | Alistipes | Alistipes |
| inops | obesi | onderdonkii | putredinis | senegalensis |
| Alistipes shahii | Alistipes | Alistipes | Alistipes | Alistipes |
| | sp. AL-1 | sp. An116 | sp. An31A | sp. An54 |
| Alistipes | Alistipes | Alistipes | Alistipes | Alistipes |
| sp. An66 | sp. CHKC1003 | sp. HGB5 | sp. Marseille-P2431 | sp. ZOR0009 |
| Alistipes | Aliterella | Alkalibacillus | Alkalibacter | Alkalibacterium |
| timonensis | atlantica | haloalkaliphilus | saccharofermentans | gilvum |
| Alkalibacterium | Alkalibacterium | Alkalibacterium | Alkalibacterium | Alkalibacterium |
| pelagium | putridalgicola | sp. 20 | sp. AK22 | subtropicum |
| Alkalibacterium | Alkaliflexus | Alkalilimnicola | Alkalimonas | Alkaliphilus |
| thalassium | imshenetskii | ehrlichii | amylolytica | metalliredigens |
| Alkaliphilus | Alkaliphilus | Alkaliphilus | Alkalispirochaeta | Alkalispirochaeta |
| oremlandii | peptidifermentans | transvaalensis | alkalica | americana |
| Alkalispirochaeta | Alkalitalea | Alkanindiges | Alkanindiges | Allisonella |
| odontotermitis | saponilacus | illinoisensis | sp. H1 | histaminiformans |
| Alloactinosynnema | Alloactinosynnema | Alloactinosynnema | Allobaculum | Allochromatium |
| album | iranicum | sp. L-07 | stercoricanis | vinosum |
| Allochromatium | Allofrancisella | Allofustis | Alloiococcus | Allokutzneria |
| warmingii | guangzhouensis | seminis | otitis | albata |
| Allokutzneria | Alloprevotella | Alloprevotella | Allosalinactinospora | Alloscardovia |
| sp. NRRL B-24872 | rava | tannerae | lopnorensis | criceti |
| Alloscardovia | Alloscardovia | Alloscardovia | alpha proteobacterium | alpha proteobacterium |
| macacae | omnicolens | sp. HMSC034E08 | AAP38 | AAP81b |
| alpha proteobacterium | alpha proteobacterium | alpha proteobacterium | alpha proteobacterium | alpha proteobacterium |
| BAL199 | H1MB114 | HIMB5 | HIMB59 | IMCC14465 |
| alpha proteobacterium | alpha proteobacterium | alpha proteobacterium | alpha proteobacterium | alpha proteobacterium |
| L41A | LLX12A | Mf 1.05b.01 | Q-1 | RS24 |
| alpha proteobacterium | Altererythrobacter | Altererythrobacter | Altererythrobacter | Altererythrobacter |
| U9-1i | atlanticus | dongtanensis | epoxidivorans | ishigakiensis |
| Altererythrobacter | Altererythrobacter | Altererythrobacter | Altererythrobacter | Altererythrobacter |
| marensis | namhicola | sp. C9-11 | sp. MH-B5 | sp. Root672 |
| Altererythrobacter | Altererythrobacter | Alteribacillus | Alteribacillus | Alteribacillus |
| troitsensis | xiamenensis | bidgolensis | iranensis | persepolensis |
| Alternaria | Alteromonadaceae | Alteromonadaceae | Alteromonadaceae | Alteromonadales |
| alternata | bacterium Bs12 | bacterium Bs31 | bacterium XY-R5 | bacterium BS08 |
| Alteromonas | Alteromonas | Alteromonas | Alteromonas | Alteromonas |
| australica | confluentis | lipolytica | macleodii | marina |
| Alteromonas | Alteromonas | Alteromonas | Alteromonas | Altibacter |
| mediterranea | naphthalenivorans | sp. ALT199 | sp. RW2A1 | lentus |
| Alysiella | Amantichitinum | Aminiphilus | Aminobacter | Aminobacter |
| crassa | ursilacus | circumscriptus | aminovorans | sp. J41 |
| Aminobacter | Aminobacterium | Aminobacterium | Aminomonas | Ammonifex |
| sp. Root100 | colombiense | mobile | paucivorans | degensii |
| Amorphus | Amphibacillus | Amphibacillus | Amphibacillus | Amphibacillus |
| coralli | jilinensis | marinus | sediminis | xylanus |
| Amphiplicatus | Amphritea | Amphritea | Amycolatopsis | Amycolatopsis |
| metriothermophilus | atlantica | japonica | alba | australiensis |
| Amycolatopsis | Amycolatopsis | Amycolatopsis | Amycolatopsis | Amycolatopsis |
| azurea | balhimycina | benzoatilytica | coloradensis | decaplanina |
| Amycolatopsis | Amycolatopsis | Amycolatopsis | Amycolatopsis | Amycolatopsis |
| halophila | japonica | jejuensis | kentuckyensis | keratiniphila |
| Amycolatopsis | Amycolatopsis | Amycolatopsis | Amycolatopsis | Amycolatopsis |
| lexingtonensis | lurida | marina | mediterranei | methanolica |
| Amycolatopsis | Amycolatopsis | Amycolatopsis | Amycolatopsis | Amycolatopsis |
| nigrescens | orientalis | pretoriensis | regifaucium | rifamycinica |
| Amycolatopsis | Amycolatopsis | Amycolatopsis | Amycolatopsis | Amycolatopsis |
| rubida | saalfeldensis | sacchari | sp. ATCC 39116 | sp. AU-G6 |
| Amycolatopsis | Amycolatopsis | Amycolatopsis | Amycolatopsis | Amycolatopsis |
| sp. H5 | sp. KNN50.9b | sp. M39 | sp. MJM2582 | taiwanensis |
| Amycolatopsis | Amycolatopsis | Amycolatopsis | Amycolatopsis | Amycolatopsis |
| thailandensis | thermoflava | tolypomycina | vancoresmycina | xylanica |
| Anabaena | Anabaena | Anabaena | Anabaena | Anabaena |
| cylindrica | sp. 90 | sp. CA = | sp. PCC 7108 | sp. WA102 |
| | | ATCC 33047 | | |
| Anaeroarcus | Anaerobacillus | Anaerobacillus | Anaerobacillus | Anaerobacillus |
| burkinensis | alkalidiazotrophicus | alkalilacustris | arseniciselenatis | macyae |
| Anaerobacillus | Anaerobiospirillum | Anaerobium | Anaerobranca | Anaerobranca |
| sp. NB2006 | succiniciproducens | acetethylicum | californiensis | gottschalkii |
| Anaerococcus | Anaerococcus | Anaerococcus | Anaerococcus | Anaerococcus |
| hydrogenalis | lactolyticus | mediterraneensis | obesiensis | pacaensis |
| Anaerococcus | Anaerococcus | Anaerococcus | Anaerococcus | Anaerococcus |
| prevotii | provencensis | rubeinfantis | senegalensis | sp. HMSC065G05 |
| Anaerococcus | Anaerococcus | Anaerococcus | Anaerococcus | Anaerococcus |
| sp. HMSC068A02 | sp. HMSC075B03 | sp. Marseille-P2143 | sp. SB3 | tetradius |
| Anaerococcus | Anaerocolumna | Anaerocolumna | Anaerocolumna | Anaerqfilum |
| vaginalis | aminovalerica | jejuensis | xylanovorans | sp. An201 |
| Anaerofustis | Anaeroglobus | Anaerolinea | Anaerolinea | Anaerolineaceae |
| stercorihominis | geminatus | thermolimosa | thermophila | bacterium oral taxon |
| | | | | 439 |
| Anaeromassilibacillus | Anaeromassilibacillus | Anaeromassilibacillus | Anaeromassilibacillus | Anaeromassilibacillus |
| senegalensis | sp. An172 | sp. An200 | sp. An250 | sp. Marseille-P3371 |
| Anaeromicrobium | Anaeromusa | Anaeromyxobacter | Anaeromyxobacter | Anaeromyxobacter |
| sediminis | acidaminophila | dehalogenans | sp. Fw109-5 | sp. K |
| Anaeromyxobacter | Anaerophaga | Anaerorhabdus | Anaerosalibacter | Anaerosalibacter |
| sp. PSR-1 | thermohalophila | furcosa | massiliensis | sp. Marseille-P3206 |
| Anaerosphaera | Anaerosphaera | Anaerosporobacter | Anaerosporomusa | Anaerostipes |
| aminiphila | sp. HMSC064C01 | mobilis | subterranea | caccae |
| Anaerostipes | Anaerostipes | Anaerostipes | Anaerostipes | Anaerostipes |
| hadrus | sp. 3_2_56FAA | sp. 494a | sp. 992a | sp. BG01 |
| Anaerotruncus | Anaerotruncus | Anaerotruncus | Anaerovibrio | Anaerovibrio |
| colihominis | rubiinfantis | sp. G3(2012) | lipolyticus | sp. JC8 |
| Anaerovibrio | Anaerovirgula | Anaerovorax | Anaplasma | Anaplasma |
| sp. RM50 | multivorans | odorimutans | centrale | marginale |
| Anaplasma | Ancylobacter | Anditalea | Andreprevotia | Andreprevotia |
| phagocytophilum | rudongensis | andensis | chitinilytica | lacus |
| Aneurinibacillus | Aneurinibacillus | Aneurinibacillus | Aneurinibacillus | Aneurinibacillus |
| aneurinilyticus | migulanus | terranovensis | thermoaerophilus | tyrosinisolvens |
| Angelakisella | Angustibacter | Anoxybacillus | Anoxybacillus | Anoxybacillus |
| massiliensis | sp. Root456 | amylolyticus | ayderensis | flavithermus |
| Anoxybacillus | Anoxybacillus | Anoxybacillus | Anoxybacillus | Anoxybacillus |
| geothermalis | kamchatkensis | mongoliensis | pushchinoensis | sp. 103 |
| Anoxybacillus | Anoxybacillus | Anoxybacillus | Anoxybacillus | Anoxybacillus |
| sp. BCO1 | sp. DT3-1 | sp. KU2-6(11) | sp. P3H1B | sp. SK3-4 |
| Anoxybacillus | Anoxybacillus | Anoxybacillus | Anoxybacillus | Antarctobacter |
| sp. UARK-01 | suryakundensis | tepidamans | thermarum | heliothermus |
| Anthococcus | Anthracocystis | Aphanizomenon | Aphanomyces | Aphanomyces |
| sp. DF1 | flocculosa | flos-aquae | astaci | invadans |
| Apibacter | Aquabacterium | Aquabacterium | Aquamicrobium | Aquamicrobium |
| mensalis | parvum | sp. NJ1 | aerolatum | defluvii |
| Aquaspirillum | Aquaspirillum | Aquifex | Aquiflexum | Aquimarina |
| serpens | sp. LM1 | aeolicus | balticum | agarivorans |
| Aquimarina | Aquimarina | Aquimarina | Aquimarina | Aquimarina |
| amphilecti | atlantica | latercula | longa | macrocephali |
| Aquimarina | Aquimarina | Aquimarina | Aquimarina | Aquimarina |
| megaterium | muelleri | paciflca | sp. RZW4-3-2 | spongiae |
| Aquimixticola | Aquimonas | Aquincola | Aquisalimonas | Aquitalea |
| soesokkakensis | voraii | tertiaricarbonis | asiatica | magntisonii |
| Aquitalea | Arabia | Arachidicoccus | Arachidicoccus | Arcanobacterium |
| pelogenes | massiliensis | rhizosphaerae | sp. BS20 | haemolyticum |
| Arcanobacterium | Arcanobacterium | Arcanobacterium | Archaeoglobus | Archaeoglobus |
| phocae | sp. S3PF19 | urinimassiliense | fulgidus | profundus |
| Archaeoglobus | Archangium | Archangium | Archangium | Arcobacter |
| veneficus | gephyra | sp. Cb G35 | violaceum | anaerophilus |
| Arcobacter | Arcobacter | Arcobacter | Arcobacter | Arcobacter |
| butzleri | cibarius | cryaerophilus | lanthieri | lekithochrous |
| Arcobacter | Arcobacter | Arcobacter | Arcobacter | Arcobacter |
| marinus | nitrofigilis | porcinus | skirrowii | sp. L |
| Arcobacter | Arcobacter | Arcobacter | Arcticibacter | Arcticibacter |
| sp. LA11 | sp. LPB0137 | thereius | eurypsychrophilus | svalbardensis |
| Ardenticatena | Arenibacter | Arenibacter | Arenibacter | Arenibacter |
| maritima | algicola | certesii | latericius | nanhaiticus |
| Arenibacter | Arenibacter | Arenibacter | Arenimonas | Arenimonas |
| palladensis | sp. AK53 | troitsensis | composti | donghaensis |
| Arenimonas | Arenimonas | Arenimonas | Arenitalea | Arhodomonas |
| malthae | metalli | oryziterrae | lutea | uipiueolei |
| Armatimonadetes | Armatimonadetes | Aromatoleum | Arsenicibacter | Arsenicicoccus |
| bacterium DC | bacterium GXS | aromaticum | rosenii | bolidensis |
| Arsenicicoccus | Arsenophonus | Arsenophonus | Arsenophonus | Arsiikibacterium |
| sp. oral | endosymbiont of | nasoniae | symbiont of | ikkense |
| taxon 190 | Nilaparvata lugens | | Lipoptena fortisetosa | |
| Arsiikibacterium | Arthrobacter | Arthrobacter | Arthrobacter | Arthrobacter |
| sp. MJ3 | agilis | alpinus | castelli | crystallopoietes |
| Arthrobacter | Arthrobacter | Arthrobacter | Arthrobacter | Arthrobacter |
| cupressi | enclensis | globiformis | koreensis | luteolus |
| Arthrobacter | Arthrobacter | Arthrobacter | Arthrobacter | Arthrobacter |
| nitrophenolicus | rhombi | sp. 131MFCol6.1 | sp. 135MFCol5.1 | sp. 162MFSha1.1 |
| Arthrobacter | Arthrobacter | Arthrobacter | Arthrobacter | Arthrobacter |
| sp. 31Cvi3.1E | sp. 31Y | sp. 35/47 | sp. 35W | sp. 49Tsu3.1M3 |
| Arthrobacter | Arthrobacter | Arthrobacter | Arthrobacter | Arthrobacter |
| sp. 9MFCol3.1 | sp. A3 | sp. ATCC | sp. B6 | sp. Br18 |
| | | 21022 | | |
| Arthrobacter | Arthrobacter | Arthrobacter | Arthrobacter | Arthrobacter |
| sp. CAL618 | sp. cf158 | sp. Edens01 | sp. EpRS66 | sp. EpRS71 |
| Arthrobacter | Arthrobacter | Arthrobacter | Arthrobacter | Arthrobacter |
| sp. EPSL27 | sp. ERGS1: 01 | sp. FB24 | sp. H14 | sp. H20 |
| Arthrobacter | Arthrobacter | Arthrobacter | Arthrobacter | Arthrobacter |
| sp. H41 | sp. H5 | sp. HG | sp. Hiyo1 | sp. HMSC06H05 |
| Arthrobacter | Arthrobacter | Arthrobacter | Arthrobacter | Arthrobacter |
| sp. HMSC08H08 | sp. I3 | sp. IHBB 11108 | sp. KBS0703 | sp. L77 |
| Arthrobacter | Arthrobacter | Arthrobacter | Arthrobacter | Arthrobacter |
| sp. Leaf137 | sp. Leaf141 | sp. Leaf234 | sp. Leaf337 | sp. Leaf69 |
| Arthrobacter | Arthrobacter | Arthrobacter | Arthrobacter | Arthrobacter |
| sp. LS16 | sp. M2012083 | sp. MA-N2 | sp. MWB30 | sp. NIO-1057 |
| Arthrobacter | Arthrobacter | Arthrobacter | Arthrobacter | Arthrobacter |
| sp. ok362 | sp. ok909 | sp. ov118 | sp. ov407 | sp. OV608 |
| Arthrobacter | Arthrobacter | Arthrobacter | Arthrobacter | Arthrobacter |
| sp. OY3WO11 | sp. P2b | sp. PAMC 25486 | sp. PAO19 | sp. QXT-31 |
| Arthrobacter | Arthrobacter | Arthrobacter | Arthrobacter | Arthrobacter |
| sp. RIT-PI-e | sp. Rue61a | sp. Soil736 | sp. Soil761 | sp. Soil762 |
| Arthrobacter | Arthrobacter | Arthrobacter | Arthrobacter | Arthrobacter |
| sp. Soil763 | sp. Soil764 | sp. Soil782 | sp. SPG23 | sp. SRS-W-1-2016 |
| Arthrobacter | Arthrobacter | Arthrobacter | Arthrobacter | Arthrobacter |
| sp. SW1 | sp. TB 23 | sp. TB 26 | sp. U41 | sp. UCD-GKA |
| Arthrobacter | Arthrobacter | Arthrobacter | Arthrobacter | Arthrobacter |
| sp. UNC362MFTsu5.1 | sp. UNCCL28 | sp. W1 | sp. YC-RL1 | sp. YN |
| Arthrobacter | Arthrobacter | Arthrobacter | Arthrobacter | Arthrobacter virus |
| sp. yr096 | sp. ZBG10 | sp. ZXY-2 | subterraneus | Mudcat |
| Arthrobacter | Arthrobotrys | Arthrospira | Arthrospira | Arthrospira |
| woluwensis | oligospora | platensis | sp. PCC 8005 | sp. TJSD091 |
| Asaccharospora | Asaia | Asaia | Asaia | Asaia |
| irregularis | astilbis | bogorensis | platycodi | prunellae |
| Asanoa | Asanoa | Ascoidea | Asinibacterium | Asinibacterium |
| hainanensis | ishikariensis | mbescens | sp. OR43 | sp. OR53 |
| Aspergillus | Aspergillus | Aspergillus | Aspergillus | Aspergillus |
| aculeatus | clavatus | fischeri | flavus | fumigatus |
| Aspergillus | Aspergillus | Aspergillus | Aspergillus | Aspergillus |
| nidulans | niger | nomius | oryzae | terreus |
| Asticcacaulis | Asticcacaulis | Asticcacaulis | Asticcacaulis | Asticcacaulis |
| benevestitus | biprosthecium | excentricus | sp. AC402 | sp. AC460 |
| Asticcacaulis | Asticcacaulis | Asticcacaulis | Atlantibacter | Atopobacter |
| sp. AC466 | sp. YBE204 | taihuensis | hermannii | phocae |
| Atopobium | Atopobium | Atopobium | Atopobium | Atopobium |
| deltae | fossor | minutum | parvulum | rimae |
| Atopobium | Atopobium | Atopobium | Atopobium | Atopobium |
| sp. BS2 | sp. BV3Ac4 | sp. HMSC064B08 | sp. ICM42b | sp. oral |
| | | | | taxon 199 |
| Atopobium | Atopobium | Atopococcus | Atopostipes | Atribacteria |
| sp. oral | vaginae | tabaci | suicloacalis | bacterium |
| taxon 810 | | | | JG10000014-F07 |
| Aurantimonas | Aurantimonas | Aurantimonas | Aurantimonas | Aurantimonas |
| coralicida | manganoxydans | sp. 22II-16-19i | sp. Leaf443 | sp. USBA 369 |
| Auraticoccus | Aureimonas | Aureimonas | Aureimonas | Aureimonas |
| monumenti | altamirensis | frigidaquae | jatrophae | phyllosphaerae |
| Aureimonas | Aureimonas | Aureimonas | Aureimonas | Aureimonas |
| sp. AU12 | sp. AU20 | sp. AU22 | sp. AU4 | sp. AU40 |
| Aureimonas | Aureimonas | Aureimonas | Aureimonas | Aureimonas |
| sp. D3 | sp. Leaf324 | sp. Leaf427 | sp. Leaf454 | sp. N4 |
| Aureimonas | Aureispira | Aureobasidium | Aureobasidium | Aureococcus |
| ureilytica | sp. CCB-QB1 | namibiae | subglaciale | anophagefferens |
| Aureococcus | Auricoccus | Auricularia | Austwickia | Avibacterium |
| anophagefferens | indicus | subglabra | chelonae | paragallinarum |
| virus | | | | |
| Azoarcus | Azoarcus | Azoarcus | Azoarcus | Azoarcus |
| olearius | sp. BH72 | sp. CIB | sp. KH32C | sp. PA01 |
| Azoarcus | Azoarcus | Azohydromonas | Azohydromonas | Azonexus |
| toluclasticus | tolulyticus | australica | lata | hydrophilus |
| Azorhizobium | Azorhizobium | Azospira | Azospirillum | Azospirillum |
| caulinodans | doebereinerae | oryzae | brasilense | halopraeferens |
| Azospirillum | Azospirillum | Azospirillum | Azospirillum | Azospirillum |
| humicireducens | lipoferum | oryzae | sp. B4 | sp. B506 |
| Azospirillum | Azotobacter | Azotobacter | Azotobacter | Azovibrio |
| thiophilum | beijerinckii | chroococcum | vinelandii | restrictus |
| Babesia | Babesia | Babesia | Babjeviella | Bacillaceae |
| bigemina | bovis | microti | inositovora | bacterium |
| | | | | MTCC 10057 |
| Bacillaceae | Bacillaceae | Bacillaceae | Bacilli | Bacillus |
| bacterium | bacterium | bacterium | bacterium | abyssalis |
| MTCC 8252 | SAOS7 | SAS-127 | VT-13-104 | |
| Bacillus | Bacillus | Bacillus | Bacillus | Bacillus |
| acidiceler | acidicola | acidiproducens | aerophilus | aidingensis |
| Bacillus | Bacillus | Bacillus | Bacillus | Bacillus |
| akibai | alcalophilus | alkalinitrilicus | alkalitelluris | altitudinis |
| Bacillus | Bacillus | Bacillus | Bacillus | Bacillus |
| alveayuensis | amyloliquefaciens | andreraoultii | anthracis | aquimaris |
| Bacillus | Bacillus | Bacillus | Bacillus | Bacillus |
| aryabhattai | atrophaeus | aurantiacus | australimaris | azotoformans |
| Bacillus | Bacillus | Bacillus | Bacillus | Bacillus |
| badius | bataviensis | beveridgei | bingmayongensis | bogoriensis |
| Bacillus | Bacillus | Bacillus | Bacillus | Bacillus |
| bombysepticus | butanolivorans | campisalis | caseinilyticus | cecembensis |
| Bacillus | Bacillus | Bacillus | Bacillus | Bacillus |
| cellulosilyticus | cereus | chagannorensis | cihuensis | circulans |
| Bacillus | Bacillus | Bacillus | Bacillus | Bacillus |
| clausii | coagulans | coahuilensis | cohnii | cytotoxicus |
| Bacillus | Bacillus | Bacillus | Bacillus | Bacillus |
| dakarensis | daliensis | dielmoensis | drentensis | eiseniae |
| Bacillus | Bacillus | Bacillus | Bacillus | Bacillus |
| encimensis | enclensis | endophyticus | farraginis | fastidiosus |
| Bacillus | Bacillus | Bacillus | Bacillus | Bacillus |
| firmus | flexus | fordii | fumarioli | gaemokensis |
| Bacillus | Bacillus | Bacillus | Bacillus | Bacillus |
| galactosidilyticus | ginsengihumi | glycinifermentans | gobiensis | gottheilii |
| Bacillus | Bacillus | Bacillus | Bacillus | Bacillus |
| halmapalus | halodurans | halosaccharovorans | halotolerans | haynesii |
| Bacillus | Bacillus | Bacillus | Bacillus | Bacillus |
| hemicellulosilyticus | hisashii | horikoshii | horneckiae | humi |
| Bacillus | Bacillus | Bacillus | Bacillus | Bacillus |
| indicus | intestinalis | jeotgali | kochii | koreensis |
| Bacillus | Bacillus | Bacillus | Bacillus | Bacillus |
| korlensis | kribbensis | krulwichiae | lentus | licheniformis |
| Bacillus | Bacillus | Bacillus | Bacillus | Bacillus |
| ligniniphilus | litoralis | loiseleuriae | lonarensis | luciferensis |
| Bacillus | Bacillus | Bacillus | Bacillus | Bacillus |
| manliponensis | mannanilyticus | marisflavi | marmarensis | massilioanorexius |
| Bacillus | Bacillus | Bacillus | Bacillus | Bacillus |
| massiliogorillae | massiliosenegalensis | megaterium | mesonae | methanolicus |
| Bacillus | Bacillus | Bacillus | Bacillus | Bacillus |
| mojavensis | muralis | mycoides | nakamurai | ndiopicus |
| Bacillus | Bacillus | Bacillus | Bacillus | Bacillus |
| nealsonii | niacini | niameyensis | novalis | obstructivus |
| Bacillus | Bacillus | Bacillus | Bacillus | Bacillus |
| oceanisediminis | okhensis | okuhidensis | oleronius | oryziterrae |
| Bacillus | Bacillus | Bacillus | Bacillus | Bacillus |
| panaciterrae | paralicheniformis | patagoniensis | persicus | praedii |
| Bacillus | Bacillus | Bacillus | Bacillus | Bacillus |
| pseudalcaliphilus | pseudofirmus | pseudomycoides | psychrosaccharolyticus | pumilus |
| Bacillus | Bacillus | Bacillus | Bacillus | Bacillus |
| rhizosphaerae | rubiinfantis | safensis | salsus | selenatarsenatis |
| Bacillus | Bacillus | Bacillus | Bacillus | Bacillus |
| shacheensis | shackletonii | siamensis | simplex | sinesaloumensis |
| Bacillus | Bacillus | Bacillus | Bacillus | Bacillus |
| smithii | solani | soli | solimangrovi | sonorensis |
| Bacillus | Bacillus | Bacillus | Bacillus | Bacillus |
| sp. 105MF | sp. 112mf | sp. 123MFChir2 | sp. 166amftsu | sp. 171095106 |
| Bacillus | Bacillus | Bacillus | Bacillus | Bacillus |
| sp. 17376 | sp. 1NLA3E | sp. 2 A 57 CT2 | sp. 278922107 | sp. 37MA |
| Bacillus | Bacillus | Bacillus | Bacillus | Bacillus |
| sp. 45385 | sp. 491mf | sp. 522 BSPC | sp. 5mfcol3.1 | sp. 72 |
| Bacillus | Bacillus | Bacillus | Bacillus | Bacillus |
| sp. 7504-2 | sp. 7586-K | sp. 7884-1 | sp. 7894-2 | sp. B14905 |
| Bacillus | Bacillus | Bacillus | Bacillus | Bacillus |
| sp. B-jedd | sp. BO | sp. CCTCCAB-2014251 | sp. CHD6a | sp. CPSM8 |
| Bacillus | Bacillus | Bacillus | Bacillus | Bacillus |
| sp. EAC | sp. EB01 | sp. FJAT-14578 | sp. FJAT-18017 | sp. FJAT-18019 |
| Bacillus | Bacillus | Bacillus | Bacillus | Bacillus |
| sp. FJAT-20673 | sp. FJAT-22058 | sp. FJAT-22090 | sp. FJAT-25496 | sp. FJAT-25509 |
| Bacillus | Bacillus | Bacillus | Bacillus | Bacillus |
| sp. FJAT-26390 | sp. FJAT-26652 | sp. FJAT-27225 | sp. FJAT-27231 | sp. FJAT-27238 |
| Bacillus | Bacillus | Bacillus | Bacillus | Bacillus |
| sp. FJAT-27245 | sp. FJAT- 27251 | sp. FJAT-27264 | sp. FJAT-27445 | sp. FJAT-27916 |
| Bacillus | Bacillus | Bacillus | Bacillus | Bacillus |
| sp. FJAT-27986 | sp. FJAT-28004 | sp. FJAT-29814 | sp. FJAT-29937 | sp. FJAT-44921 |
| Bacillus | Bacillus | Bacillus | Bacillus | Bacillus |
| sp. FMQ74 | sp. GZT | sp. HMSC76G11 | sp. 1-2 | sp. J13 |
| Bacillus | Bacillus | Bacillus | Bacillus | Bacillus |
| sp. J33 | sp. J37 | sp. JCM 19034 | sp. JCM 19035 | sp. JCM 19041 |
| Bacillus | Bacillus | Bacillus | Bacillus | Bacillus |
| sp. JCM 19047 | sp. JEM-1 | sp. JFL15 | sp. JS | sp. KbaL1 |
| Bacillus | Bacillus | Bacillus | Bacillus | Bacillus |
| sp. Leaf406 | sp. Leaf49 | sp. Leaf75 | sp. LF1 | sp. LK2 |
| Bacillus | Bacillus | Bacillus | Bacillus | Bacillus |
| sp. LL01 | sp. LYLB4 | sp. M 2-6 | sp. m3-13 | sp. Marseille-P2366 |
| Bacillus | Bacillus | Bacillus | Bacillus | Bacillus |
| sp. Marseille-P2384 | sp. MB2021 | sp. MB353a | sp. MB366 | sp. MKU004 |
| Bacillus | Bacillus | Bacillus | Bacillus | Bacillus |
| sp. MRMR6 | sp. MSP13 | sp. MSP5.4 | sp. MUM 116 | sp. MUM 13 |
| Bacillus | Bacillus | Bacillus | Bacillus | Bacillus |
| sp. N24 | sp. NC2-31 | sp. NEAU-cbsb5 | sp. NH11B | sp. NH24A2 |
| Bacillus | Bacillus | Bacillus | Bacillus | Bacillus |
| sp. NSP22.2 | sp. NSP9.1 | sp. OG2 | sp. OK048 | sp. OK838 |
| Bacillus | Bacillus | Bacillus | Bacillus | Bacillus |
| sp. OV166 | sp. OV194 | sp. OV322 | sp. OxB-1 | sp. REN51N |
| Bacillus | Bacillus | Bacillus | Bacillus | Bacillus |
| sp. Root147 | sp. Root239 | sp. Root920 | sp. RRD69 | sp. RU2C |
| Bacillus | Bacillus | Bacillus | Bacillus | Bacillus |
| sp. S1-R1J2-FB | sp. S1-R2T1- FB | sp. S1-R4H1-FB | sp. S1-R5C1-FB | sp. S2-R3J1-FB-BA1 |
| Bacillus | Bacillus | Bacillus | Bacillus | Bacillus |
| sp. SA1-12 | sp. SA5d-4 | sp. SDL11 | sp. SG-1 | sp. SJS |
| Bacillus | Bacillus | Bacillus | Bacillus | Bacillus |
| sp. Soil745 | sp. Soil768D1 | sp. TS-2 | sp. UNC125MFCrub1.1 | sp. UNC322MFChir4.1 |
| Bacillus | Bacillus | Bacillus | Bacillus | Bacillus |
| sp. UNC41MFS5 | sp. UNC437CL72CviS29 | sp. UNC438CL73TsuS30 | sp. UNCCL81 | sp. URHB0009 |
| Bacillus | Bacillus | Bacillus | Bacillus | Bacillus |
| sp. VT-16-64 | sp. WP8 | sp. X1 (2014) | sp. X2(2017) | sporothermodurans |
| Bacillus | Bacillus | Bacillus | Bacillus | Bacillus |
| stratosphericus | subterraneus | subtilis | swezeyi | tequilensis |
| Bacillus | Bacillus | Bacillus | Bacillus | Bacillus |
| testis | thermoamylovorans | thermotolerans | thuringiensis | timonensis |
| Bacillus | Bacillus | Bacillus | Bacillus | Bacillus |
| toyonensis | trypoxylicola | tuaregi | vallismortis | velezensis |
| Bacillus | Bacillus | Bacillus | Bacillus | Bacillus |
| vietnamensis | vireti | virus G | virus Hakuna | wakoensis |
| Bacillus | Bacillus | Bacillus | Bacillus | Bacillus |
| weihaiensis | weihenstephanensis | wiedmannii | wudalianchiensis | xerothermodurans |
| Bacillus | Bacillus | Bacterioplanes | Bacteriovorax | Bacteriovorax |
| xiamenensis | zhangzhouensis | sanyensis | sp. BAL6_X | sp. BSW11_IV |
| Bacteriovorax | Bacteriovorax | bacterium 2013Arg42i | bacterium 2013Ark19i | bacterium 336/3 |
| sp. DB6_IX | sp. Seq25_V | | | |
| bacterium A37T11 | bacterium A52C2 | bacterium A7P-90m | bacterium | bacterium |
| | | | endosymbiont of | endosymbiont of |
| | | | Mortierella elongata | Pedicinus |
| | | | FMR23-6 | badii |
| bacterium JKG1 | bacterium LF-3 | bacterium MS4 | bacterium OL-1 | bacterium SIT5 |
| bacterium SIT6 | Bacteroidales | Bacteroidales | Bacteroidales | Bacteroidales |
| | bacterium 6E | bacterium Barb4 | bacterium Barb6 | bacterium Barb6XT |
| Bacteroidales | Bacteroidales | Bacteroidales | Bacteroidales | Bacteroidales |
| bacterium CF | bacterium KA00251 | bacterium KA00344 | bacterium KHT7 | bacterium IVCE2004 |
| Bacteroidales | Bacteroides | Bacteroides | Bacteroides | Bacteroides |
| bacterium WCE2008 | acidifaciens | barnesiae | caccae | caecimuris |
| Bacteroides | Bacteroides | Bacteroides | Bacteroides | Bacteroides |
| cellulosilyticus | clarus | coprocola | coprophilus | coprosuis |
| Bacteroides | Bacteroides | Bacteroides | Bacteroides | Bacteroides |
| dorei | eggerthii | faecichinchillae | faecis | finegoldii |
| Bacteroides | Bacteroides | Bacteroides | Bacteroides | Bacteroides |
| fluxiis | fragilis | gallinarum | graminisolvens | helcogenes |
| Bacteroides | Bacteroides | Bacteroides | Bacteroides | Bacteroides |
| ihuae | intestinalis | luti | massiliensis | mediterraneensis |
| Bacteroides | Bacteroides | Bacteroides | Bacteroides | Bacteroides |
| neonati | nordii | oleiciplenus | ovatus | paurosaccharolyticus |
| Bacteroides | Bacteroides | Bacteroides | Bacteroides | Bacteroides |
| plebeius | propionicifaciens | pyogenes | reticulotermitis | salanitronis |
| Bacteroides | Bacteroides | Bacteroides | Bacteroides | Bacteroides |
| salyersiae | sartorii | sp. 1_1_30 | sp. 1_1_6 | sp. 14(A) |
| Bacteroides | Bacteroides | Bacteroides | Bacteroides | Bacteroides |
| sp. 2_1_16 | sp. 2_1_22 | sp. 2_1_33B | sp. 2_2_4 | sp. 3_1_13 |
| Bacteroides | Bacteroides | Bacteroides | Bacteroides | Bacteroides |
| sp. 3_1_19 | sp. 3_1_23 | sp. 3_1_33FAA | sp. 3_1_40A | sp. 3_2_5 |
| Bacteroides | Bacteroides | Bacteroides | Bacteroides | Bacteroides |
| sp. 4_1_36 | sp. 4_3_47FAA | sp. 9_1_42FAA | sp. An19 | sp. An269 |
| Bacteroides | Bacteroides | Bacteroides | Bacteroides | Bacteroides |
| sp. An279 | sp. An322 | sp. An51A | sp. AR20 | sp. D1 |
| Bacteroides | Bacteroides | Bacteroides | Bacteroides | Bacteroides |
| sp. D2 | sp. D20 | sp. D22 | sp. HMSC067B03 | sp. HMSC068A09 |
| Bacteroides | Bacteroides | Bacteroides | Bacteroides | Bacteroides |
| sp. HMSC073E02 | sp. HPS0048 | sp. Marseille-P2653 | sp. Marseille-P3108 | sp. Marseille-P3132 |
| Bacteroides | Bacteroides | Bacteroides | Bacteroides | Bacteroides |
| sp. Marseille-P3166 | sp. Marseille-P3208T | sp. UW | stercorirosoris | stercoris |
| Bacteroides | Bacteroides | Bacteroides | Bacteroides | Bacteroides |
| thetaiotaomicron | timonensis | uniformis | vulgatus | xylanisolvens |
| Bacteroidetes | Bacteroidetes | Balneatrix | Balneola | Balneola |
| bacterium | oral taxon 274 | alpica | sp. EhC07 | vulgaris |
| oral taxon 272 | | | | |
| Bariatricus | Barnesiella | Barnesiella | Barnesiella | Barnesiella |
| massiliensis | intestinihominis | sp. An22 | sp. An55 | viscericola |
| Bartonella | Bartonella | Bartonella | Bartonella | Bartonella |
| alsatica | ancashensis | apis | australis | bacilliformis |
| Bartonella | Bartonella | Bartonella | Bartonella | Bartonella |
| birtlesii | bovis | clarridgeiae | doshiae | elizabethae |
| Bartonella | Bartonella | Bartonella | Bartonella | Bartonella |
| florencae | grahamii | henselae | koehlerae | melophagi |
| Bartonella | Bartonella | Bartonella | Bartonella | Bartonella |
| queenslandensis | quintana | rattaustraliani | rattimassiliensis | senegalensis |
| Bartonella | Bartonella | Bartonella | Bartonella | Bartonella |
| sp. AR 15-3 | sp. DB5-6 | sp. WD12.1 | tamiae | taylorii |
| Bartonella | Bartonella | Bartonella | Basfia | Basilea |
| tribocorum | vinsonii | washoensis | succiniciproducens | psittacipulmonis |
| Bathymodiolus | Bathymodiolus | Bathymodiolus | Bathymodiolus | Batrachochytrium |
| azoricus | platifrons | septemdierum | thermophilus | dendrobatidis |
| thioautotrophic gill | methanotrophic gill | thioautotrophic gill | thioautotrophic gill | |
| symbiont | symbiont | symbiont | symbiont | |
| Baudoinia | Bauldia | Bavariicoccus | Bdellovibrio | Bdellovibrio |
| panamericana | litoralis | seileri | bacteriovorus | exovorus |
| Bdellovibrio | BeAn 58058 virus | Beauveria | Beduini | Beggiatoa |
| sp. SKB1291214 | | bassiana | massiliensis | alba |
| Beggiatoa | Beijerinckia | Beijerinckia | Beijerinckia | Belliella |
| leptomitiformis | indica | mobilis | sp. 28- | baltica |
| | | | YEA-48 | |
| Belliella | Belliella | Bellilinea | Belnapia | Belnapia |
| buryatensis | pelovolcani | caldifistulae | moabensis | rosea |
| Belnapia | Bergeriella | Bergeyella | Bermanella | Bernardetia |
| sp. F-4-1 | denitrificans | zoohelcum | marisrubri | litoralis |
| beta proteobacterium | beta proteobacterium | beta proteobacterium | beta proteobacterium | beta proteobacterium |
| AAP51 | AAP99 | CB | KB13 | L13 |
| Betaproteobacteria | Betaproteobacteria | Beutenbergia | Bhargavaea | Bhargavaea |
| bacterium GR16-43 | bacterium MOLA814 | cavernae | beijingensis | cecembensis |
| Bhargavaea | Bibersteinia | Bifidobacterium | Bifidobacterium | Bifidobacterium |
| ginsengi | trehalosi | actinocoloniiforme | adolescentis | aesculapii |
| Bifidobacterium | Bifidobacterium | Bifidobacterium | Bifidobacterium | Bifidobacterium |
| angulatum | animalis | aquikefiri | asteroides | biavatii |
| Bifidobacterium | Bifidobacterium | Bifidobacterium | Bifidobacterium | Bifidobacterium |
| bifidum | bohemicum | bombi | boum | breve |
| Bifidobacterium | Bifidobacterium | Bifidobacterium | Bifidobacterium | Bifidobacterium |
| callitrichos | catenulatum | choerinum | commune | coryneforme |
| Bifidobacterium | Bifidobacterium | Bifidobacterium | Bifidobacterium | Bifidobacterium |
| crudilactis | cuniculi | dentium | eulemuris | gallicum |
| Bifidobacterium | Bifidobacterium | Bifidobacterium | Bifidobacterium | Bifidobacterium |
| gallinarum | hapali | indicium | kashiwanohense | lemurum |
| Bifidobacterium | Bifidobacterium | Bifidobacterium | Bifidobacterium | Bifidobacterium |
| longum | magnum | merycicum | minimum | mongoliense |
| Bifidobacterium | Bifidobacterium | Bifidobacterium | Bifidobacterium | Bifidobacterium |
| moukalabense | myosotis | pseudocatenulatum | pseudolongum | psychraerophilum |
| Bifidobacterium | Bifidobacterium | Bifidobacterium | Bifidobacterium | Bifidobacterium |
| pullorum | reuteri | ruminantium | saeculare | saguini |
| Bifidobacterium | Bifidobacterium | Bifidobacterium | Bifidobacterium | Bifidobacterium |
| scardovii | sp. 12_1_47BFAA | sp. 7101 | sp. A11 | sp. AGR2158 |
| Bifidobacterium | Bifidobacterium | Bifidobacterium | Bifidobacterium | Bifidobacterium |
| sp. MSTE12 | sp. Tam10B | stellenboschense | subtile | thermacidophilum |
| Bifidobacterium | Bifidobacterium | Bifidobacterium | Bigelowiella | Bilophila |
| thermophilum | tissieri | tsurumiense | natans | sp. 4 1 30 |
| Bilophila | Bipolaris | Bipolaris | Bipolaris | Bipolaris |
| wadsworthia | maydis | oryzae | sorokiniana | victoriae |
| Bipolaris | Bittarella | Bizionia | Bizionia | Bizionia |
| zeicola | massiliensis | argentinensis | echini | paragorgiae |
| Bizionia | Blastochloris | Blastococcus | Blastococcus | Blastococcus |
| sp. APA-3 | viridis | endophyticus | saxobsidens | sp. DSM 44205 |
| Blastococcus | Blastococcus | Blastococcus | Blastococcus | Blastococcus |
| sp. DSM | sp. DSM | sp. DSM | sp. DSM | sp. URHD0036 |
| 44268 | 44272 | 46786 | 46838 | |
| Blastocystis | Blastocystis | Blastomonas | Blastomonas | Blastomonas |
| hominis | sp. subtype 4 | sp. AAP25 | sp. AAP53 | sp. CCH1-A6 |
| Blastomonas | Blastomonas | Blastomyces | Blastopirellula | Blattabacterium |
| sp. CCH2-A2 | sp. RAC04 | gilchristii | marina | cuenoti |
| Blattabacterium | Blattabacterium | Blattabacterium | Blattabacterium | Blattabacterium |
| punctulatus | sp. (Blaberus | sp. (Blattella | sp. (Mastotermes | sp. (Nauphoeta |
| | giganteus) | germanica) | darwiniensis) | cinerea) |
| Blattabacterium | Blautia | Blautia | Blautia | Blautia |
| sp. (Periplaneta | coccoides | hansenii | hydrogenotrophica | massiliensis |
| americana) | | | | |
| Blautia | Blautia | Blautia | Blautia | Blautia |
| obeum | producta | schinkii | sp. An249 | sp. An46 |
| Blautia | Blautia | Blautia | Blautia | Blautia |
| sp. An81 | sp. KB1 | sp. KLE 1732 | sp. Marseille-P2398 | sp. Marseille-P3087 |
| Blautia | Blautia | Blautia | Blautia | Blochmannia |
| sp. Marseille-P3201T | sp. SF-50 | sp. YL58 | wexlerae | endosymbiont of |
| | | | | Camponotus |
| | | | | (Colobopsis) |
| | | | | obliquus |
| Blochmannia | Bombella | Bombiscardovia | Bordetella | Bordetella |
| endosymbiont of | intestini | coagulans | ansorpii | avium |
| Polyrhachis | | | | |
| (Hedomyrma) | | | | |
| turneri | | | | |
| Bordetella | Bordetella | Bordetella | Bordetella | Bordetella |
| bronchialis | bronchiseptica | flabilis | genomosp. 1 | genomosp. 10 |
| Bordetella | Bordetella | Bordetella | Bordetella | Bordetella |
| genomosp. 11 | genomosp. 12 | genomosp. 13 | genomosp. 2 | genomosp. 4 |
| Bordetella | Bordetella | Bordetella | Bordetella | Bordetella |
| genomosp. 5 | genomosp. 6 | genomosp. 7 | genomosp. 8 | genomosp. 9 |
| Bordetella | Bordetella | Bordetella | Bordetella | Bordetella |
| hinzii | holmesii | parapertussis | pertussis | petrii |
| Bordetella | Bordetella | Bordetella | Bordetella | Bordetella |
| pseudohinzii | sp. FB-8 | sp. H567 | sp. N | trematum |
| Borrelia | Borrelia | Borrelia | Borrelia | Borrelia |
| anserina | hermsii | mayonii | miyamotoi | parkeri |
| Borrelia | Borrelia | Borrelia | Borreliella | Borreliella |
| persica | recurrentis | turicatae | afzelii | bissettii |
| Borreliella | Borreliella | Borreliella | Borreliella | Bosea |
| burgdorferi | garinii | spielmanii | valaisiana | lupini |
| Bosea | Bosea | Bosea | Bosea | Bosea |
| robiniae | sp. 117 | sp. AAP35 | sp. AS-1 | sp. BIWAKO-01 |
| Bosea | Bosea | Bosea | Bosea | Bosea |
| sp. DSM 18164 | sp. LC85 | sp. Leaf344 | sp. OK403 | sp. PAMC 26642 |
| Bosea | Bosea | Bosea | Bosea | Bosea |
| sp. RAC05 | sp. Root381 | sp. Root483D1 | sp. Root670 | sp. TND4EK4 |
| Bosea | Bosea | Bosea | Bosea | Boseongicola |
| sp. UNC402CLCol | sp. WAO | thiooxidans | vaviloviae | aestuarii |
| Botrytis | Brachybacterium | Brachybacterium | Brachybacterium | Brachybacterium |
| cinerea | faecium | massiliense | muris | nesterenkovii |
| Brachybacterium | Brachybacterium | Brachybacterium | Brachybacterium | Brachybacterium |
| paraconglomeratum | phenoliresistens | sp. HMSC06H03 | sp. P6-10-X1 | sp. sponge |
| Brachybacterium | Brachybacterium | Brachybacterium | Brachymonas | Brachymonas |
| sp. SW0106-09 | sp. VR2415 | squiliarum | chironomi | denitrificans |
| Brachyspira | Brachyspira | Brachyspira | Brachyspira | Brachyspira |
| alvinipulli | hampsonii | hyodysenteriae | innocens | intermedia |
| Brachyspira | Brachyspira | Brachyspira | Brackiella | Bradyrhizobiaceae |
| murdochii | pilosicoli | suanatina | oedipodis | bacterium SG-6C |
| Bradyrhizobium | Bradyrhizobium | Bradyrhizobium | Bradyrhizobium | Bradyrhizobium |
| arachidis | canariense | diazoefficiens | elkanii | embrapense |
| Bradyrhizobium | Bradyrhizobium | Bradyrhizobium | Bradyrhizobium | Bradyrhizobium |
| erythrophlei | icense | japonicum | jicamae | lablabi |
| Bradyrhizobium | Bradyrhizobium | Bradyrhizobium | Bradyrhizobium | Bradyrhizobium |
| liaoningense | manausense | mercantei | neotropicale | oligotrophicum |
| Bradyrhizobium | Bradyrhizobium | Bradyrhizobium | Bradyrhizobium | Bradyrhizobium |
| ottawaense | pachyrhizi | paxllaeri | retamae | sp. |
| Bradyrhizobium | Bradyrhizobium | Bradyrhizobium | Bradyrhizobium | Bradyrhizobium |
| sp. 39S1MB | sp. 85S1MB | sp. Aila-2 | sp. ARR65 | sp. AS23.2 |
| Bradyrhizobium | Bradyrhizobium | Bradyrhizobium | Bradyrhizobium | Bradyrhizobium |
| sp. AT1 | sp. BR 10245 | sp. BR 10303 | sp. BR10280 | sp. BTAi1 |
| Bradyrhizobium | Bradyrhizobium | Bradyrhizobium | Bradyrhizobium | Bradyrhizobium |
| sp. CCBAU 15544 | sp. CCBAU 15635 | sp. CCBAU 43298 | sp. CCGE-LA001 | sp. CCH10-C7 |
| Bradyrhizobium | Bradyrhizobium | Bradyrhizobium | Bradyrhizobium | Bradyrhizobium |
| sp. CCH4-A6 | sp. CCH5-F6 | sp. cf659 | sp. Cp5.3 | sp. DOA1 |
| Bradyrhizobium | Bradyrhizobium | Bradyrhizobium | Bradyrhizobium | Bradyrhizobium |
| sp. DOA9 | sp. Ec3.3 | sp. err11 | sp. G22 | sp. Gha |
| Bradyrhizobium | Bradyrhizobium | Bradyrhizobium | Bradyrhizobium | Bradyrhizobium |
| sp. Ghvi | sp. Leaf396 | sp. LMTR 3 | sp. LTSP849 | sp. LTSP857 |
| Bradyrhizobium | Bradyrhizobium | Bradyrhizobium | Bradyrhizobium | Bradyrhizobium |
| sp. LTSP885 | sp. LTSPM299 | sp. NAS80.1 | sp. NAS96.2 | sp. NFR13 |
| Bradyrhizobium | Bradyrhizobium | Bradyrhizobium | Bradyrhizobium | Bradyrhizobium |
| sp. OK095 | sp. ORS 278 | sp. ORS 285 | sp. ORS 375 | sp. R5 |
| Bradyrhizobium | Bradyrhizobium | Bradyrhizobium | Bradyrhizobium | Bradyrhizobium |
| sp. Rc2d | sp. Rc3b | sp. S23321 | sp. STM 3809 | sp. STM 3843 |
| Bradyrhizobium | Bradyrhizobium | Bradyrhizobium | Bradyrhizobium | Bradyrhizobium |
| sp. th.b2 | sp. Tv2a-2 | sp. UFLA 03-321 | sp. URHA0002 | sp. URHA0013 |
| Bradyrhizobium | Bradyrhizobium | Bradyrhizobium | Bradyrhizobium | Bradyrhizobium |
| sp. URHD0069 | sp. WSM1253 | sp. WSM1417 | sp. WSM1743 | sp. WSM2254 |
| Bradyrhizobium | Bradyrhizobium | Bradyrhizobium | Bradyrhizobium | Bradyrhizobium |
| sp. WSM2793 | sp. WSM3983 | sp. WSM4349 | sp. WSM471 | sp. YR681 |
| Bradyrhizobium | Bradyrhizobium | Bradyrhizobium | Bradyrhizohium | Brenneria |
| stylosanthis | valentinum | viridifuturi | yuanmingense | goodwinii |
| Brenneria | Brevefilum | Brevibacillus | Brevibacillus | Brevibacillus |
| sp. EniD312 | fermentans | agri | borstelensis | brevis |
| Brevibacillus | Brevibacillus | Brevibacillus | Brevibacillus | Brevibacillus |
| choshinensis | formosus | laterosporus | massiliensis | panacihumi |
| Brevibacillus | Brevibacillus | Brevibacillus | Brevibacillus | Brevibacillus |
| parabrevis | reuszeri | sp. BC25 | sp. CF112 | sp. Leaf182 |
| Brevibacillus | Brevibacillus | Brevibacillus | Brevibacillus | Brevibacterium |
| sp. OK042 | sp. SKDU10 | sp. WF146 | thermoruber | album |
| Brevibacterium | Brevibacterium | Brevibacterium | Brevibacterium | Brevibacterium |
| casei | epidermidis | linens | mcbrellneri | ravenspurgense |
| Brevibacterium | Brevibacterium | Brevibacterium | Brevibacterium | Brevibacterium |
| sandarakinum | senegalense | siliguriense | sp. HMSC063G07 | sp. HMSC07C04 |
| Brevibacterium | Brevibacterium | Brevibacterium | Brevibacterium | Brevibacterium |
| sp. HMSC08F02 | sp. HMSC22B09 | sp. HMSC24B04 | sp. VCM10 | yomogidense |
| Brevinema | Brevundimonas | Brevundimonas | Brevundimonas | Brevundimonas |
| andersonii | abyssalis | aveniformis | bacteroides | diminuta |
| Brevundimonas | Brevundimonas | Brevundimonas | Brevundimonas | Brevundimonas |
| naejangsanensis | nasdae | sp. 374 | sp. AAP58 | sp. BAL3 |
| Brevundimonas | Brevundimonas | Brevundimonas | Brevundimonas | Brevundimonas |
| sp. DS20 | sp. EAKA | sp. GW460-12-10-14-LB2 | sp. KM4 | sp. Leaf168 |
| Brevundimonas | Brevundimonas | Brevundimonas | Brevundimonas | Brevundimonas |
| sp. Leaf280 | sp. Leaf363 | sp. LM2 | sp. Root1279 | sp. Root1423 |
| Brevundimonas | Brevundimonas | Brevundimonas | Brevundimonas | Brevundimonas |
| sp. Root608 | sp. SH203 | sp. ZS04 | subvibrioides | vesicularis |
| Brevundimonas | Brochothrix | Brochothrix | Brochothrix | Brucella |
| viscosa | campestris | phage NF5 | thermosphacta | abortus |
| Brucella | Brucella | Brucella | Brucella | Brucella |
| inopinata | melitensis | neotomae | ovis | sp. 09RB8471 |
| Brucella | Brucella | Brucella | Brucella | Brucella |
| sp. 09RB8910 | sp. 141012304 | sp. B13-0095 | sp. BO2 | sp. CMUL 026 |
| Brucella | Brucella | Brucella | Bryobacter | Buchnera |
| sp. NF 2653 | suis | vulpis | aggregatus | aphidicola |
| Budvicia | Bulleidia | Burkholderia | Burkholderia | Burkholderia |
| aquatica | extructa | ambifaria | anthina | arationis |
| Burkholderia | Burkholderia | Burkholderia | Burkholderia | Burkholderia |
| arvi | calidae | catudaia | cenocepacia | cepacia |
| Burkholderia | Burkholderia | Burkholderia | Burkholderia | Burkholderia |
| concitans | contaminans | diffusa | dolosa | fortuita |
| Burkholderia | Burkholderia | Burkholderia | Burkholderia | Burkholderia |
| gladioli | glebae | glumae | hypogeia | lata |
| Burkholderia | Burkholderia | Burkholderia | Burkholderia | Burkholderia |
| latens | mallei | metallica | multivorans | oklahomensis |
| Burkholderia | Burkholderia | Burkholderia | Burkholderia | Burkholderia |
| paludis | pedi | peredens | plantarii | pseudomallei |
| Burkholderia | Burkholderia | Burkholderia | Burkholderia | Burkholderia |
| pseudomultivorans | ptereochthonis | pyrrocinia | seminalis | sp. 2002721687 |
| Burkholderia | Burkholderia | Burkholderia | Burkholderia | Burkholderia |
| sp. 9120 | sp. A1 | sp. A2 | sp. A27 | sp. A9 |
| Burkholderia | Burkholderia | Burkholderia | Burkholderia | Burkholderia |
| sp. ABCPW 11 | sp. ABCPW 14 | sp. AU15512 | sp. AU16741 | sp. AU17325 |
| Burkholderia | Burkholderia | Burkholderia | Burkholderia | Burkholderia |
| sp. AU17457 | sp. AU27893 | sp. AU31280 | sp. AU31652 | sp. AU33423 |
| Burkholderia | Burkholderia | Burkholderia | Burkholderia | Burkholderia |
| sp. AU33803 | sp. AU4i | sp. AU6039 | sp. b13 | sp. b14 |
| Burkholderia | Burkholderia | Burkholderia | Burkholderia | Burkholderia |
| sp. BDU18 | sp. BDU19 | sp. BDU5 | sp. BDU8 | sp. Bk |
| Burkholderia | Burkholderia | Burkholderia | Burkholderia | Burkholderia |
| sp. Bp7605 | sp. BT03 | sp. CAMPA 1040 | sp. CCGE1001 | sp. CCGE1002 |
| Burkholderia | Burkholderia | Burkholderia | Burkholderia | Burkholderia |
| sp. CCGE1003 | sp. CF099 | sp. CF145 | sp. Ch1-1 | sp. E7m39 |
| Burkholderia | Burkholderia | Burkholderia | Burkholderia | Burkholderia |
| sp. E7m48 | sp. FL-7-2-10-S1-D7 | sp. GAS332 | sp. H160 | sp. HI2500 |
| Burkholderia | Burkholderia | Burkholderia | Burkholderia | Burkholderia |
| sp. H12714 | sp. H12761 | sp. JPY251 | sp. JPY347 | sp. JS23 |
| Burkholderia | Burkholderia | Burkholderia | Burkholderia | Burkholderia |
| sp. K24 | sp. KK1 | sp. LA-2-3-30-S1-D2 | sp. Leaf177 | sp. lig30 |
| Burkholderia | Burkholderia | Burkholderia | Burkholderia | Burkholderia |
| sp. LMG 28154 | sp. MSMB0265 | sp. MSMB0852 | sp. MSMB0856 | sp. MSMB1072 |
| Burkholderia | Burkholderia | Burkholderia | Burkholderia | Burkholderia |
| sp. MSMB1459WGS | sp. MSMB1826 | sp. MSMB1835 | sp. MSMB2041 | sp. OLGA172 |
| Burkholderia | Burkholderia | Burkholderia | Burkholderia | Burkholderia |
| sp. PAMC 26561 | sp. PAMC 28687 | sp. RF2-non_BP3 | sp. RF4-BP95 | sp. RPE64 |
| Burkholderia | Burkholderia | Burkholderia | Burkholderia | Burkholderia |
| sp. RPE67 | sp. SRS-W-2-2016 | sp. TNe-862 | sp. TSV85 | sp. TSV86 |
| Burkholderia | Burkholderia | Burkholderia | Burkholderia | Burkholderia |
| sp. URHA0054 | sp. USMm B20 | sp. UYPR1.413 | sp. WP9 | sp. WSM2230 |
| Burkholderia | Burkholderia | Burkholderia | Burkholderia | Burkholderia |
| sp. WSM2232 | sp. WSM4176 | sp. YI23 | sp. YR277 | sp. yr281 |
| Burkholderia | Burkholderia | Burkholderia | Burkholderia | Burkholderia |
| stabilis | stagnalis | temeraria | territorii | thailandensis |
| Burkholderia | Burkholderia | Burkholderia | Burkholderiaceae | Burkholderiaceae |
| turbans | ubonensis | vietnamiensis | bacterium 16 | bacterium 26 |
| Burkholderiales | Burkholderiales | Burkholderiales | Burkholderiales | Buttiauxella |
| bacterium 1_1_47 | bacterium GJ-E10 | bacterium JOSHI_001 | bacterium YL45 | agrestis |
| Buttiauxella | Buttiauxella | Buttiauxella | Buttiauxella | Butyricicoccus |
| brennerae | ferragutiae | gaviniae | noackiae | desmolans |
| Butyricicoccus | Butyricicoccus | Butyricimonas | Butyricimonas | Butyricimonas |
| pullicaecorum | sp. BB10 | sp. An62 | sp. Marseille-P2440 | synergistica |
| Butyricimonas | Butyrivibrio | Butyrivibrio | Butyrivibrio | Butyrivibrio |
| virosa | crossotus | fibrisolvens | hungatei | proteoclasticus |
| Butyrivibrio | Butyrivibrio | Butyrivibrio | Butyrivibrio | Butyrivibrio |
| sp. AC2005 | sp. AD3002 | sp. AE2005 | sp. AE2015 | sp. AE2032 |
| Butyrivibrio | Butyrivibrio | Butyrivibrio | Butyrivibrio | Butyrivibrio |
| sp. AE3003 | sp. AE3004 | sp. AE3006 | sp. AE3009 | sp. FC2001 |
| Butyrivibrio | Butyrivibrio | Butyrivibrio | Butyrivibrio | Butyrivibrio |
| sp. FCS006 | sp. FCS014 | sp. INlla14 | sp. INlla16 | sp. INlla18 |
| Butyrivibrio | Butyrivibrio | Butyrivibrio | Butyrivibrio | Butyrivibrio |
| sp. INlla21 | sp. LB2008 | sp. LC3010 | sp. M55 | sp. MB2005 |
| Butyrivibrio | Butyrivibrio | Butyrivibrio | Butyrivibrio | Butyrivibrio |
| sp. MC2013 | sp. MC2021 | sp. NC2002 | sp. NC2007 | sp. NC3005 |
| Butyrivibrio | Butyrivibrio | Butyrivibrio | Butyrivibrio | Butyrivibrio |
| sp. ob235 | sp. TB | sp. VCB2001 | sp. FCB2006 | sp. VCD2006 |
| Butyrivibrio | Butyrivibrio | Butyrivibrio | Butyrivibrio | Butyrivibrio |
| sp. WCD2001 | sp. WCD3002 | sp. WCE2006 | sp. XBB1001 | sp. XPD2002 |
| Butyrivibrio | Butyrivibrio | Caballeronia | Caballeronia | Caballeronia |
| sp. XPD2006 | sp. YAB3001 | choica | cordobensis | glathei |
| Caballeronia | Caballeronia | Caballeronia | Caballeronia | Caballeronia |
| grimmiae | humi | jiangsuensis | megalochromosomata | mineralivorans |
| Caballeronia | Caballeronia | Caballeronia | Caballeronia | Caballeronia |
| sordidicola | telluris | terrestris | udeis | zhejiangensis |
| Caecibacter | Caedibacter | Caenibacillus | Caenimonas | Caenispirillum |
| massiliensis | varicaedens | caldisaponilyticus | sp. SL110 | salinarum |
| Caldalkalibacillus | Caldanaerobacter | Caldanaerobius | Caldanaerobius | Caldanaerovirga |
| thermarum | subterraneus | fijiensis | polysaccharolyticus | acetigignens |
| Calderihabitans | Caldibacillus | Caldicellulosiruptor | Caldicellulosiruptor | Caldicellulosiruptor |
| maritimus | debilis | acetigenus | bescii | hydrothermalis |
| Caldicellulosiruptor | Caldicellulosiruptor | Caldicellulosiruptor | Caldicellulosiruptor | Caldicellulosiruptor |
| kristjanssonii | kronotskyensis | lactoaceticus | obsidiansis | owensensis |
| Caldicellulosiruptor | Caldicellulosiruptor | Caldicellulosiruptor | Caldicellulosiruptor | Caldicoprobacter |
| saccharolyticus | sp. F32 | sp. Rt8.B8 | sp. Wai35.B1 | faecalis |
| Caldicoprobacter | Caldilinea | Caldimonas | Caldimonas | Caldisalinibacter |
| oshimai | aerophila | manganoxidans | taiwanensis | kiritimatiensis |
| Caldisericum | Caldisphaera | Calditerricola | Calditerrivibrio | Caldithrix |
| exile | lagunensis | satsumensis | nitroreducens | abyssi |
| Caldivirga | Caloramator | Caloramator | Caloramator | Caloramator |
| sp. MU80 | australicus | mitchellensis | proteoclasticus | quimbayensis |
| Caloramator | Caloranaerobacter | Caloranaerobacter | Caloranaerobacter | Calothrix |
| sp. ALD01 | azorensis | ferrireducens | sp. TR13 | parietina |
| Calothrix | Calothrix | Calothrix | Calothrix | Calothrix |
| rhizosoleniae | sp. 336/3 | sp. HK-06 | sp. PCC 7103 | sp. PCC 7507 |
| Calyptogena | Caminibacter | Caminicella | Campylobacter | Campylobacter |
| okutanii | mediatlanticus | sporogenes | avium | coli |
| thioautotrophic gill | | | | |
| symbiont | | | | |
| Campylobacter | Campylobacter | Campylobacter | Campylobacter | Campylobacter |
| concisus | corcagiensis | cuniculorum | ciirvus | fetus |
| Campylobacter | Campylobacter | Campylobacter | Campylobacter | Campylobacter |
| gracilis | helveticus | hepaticus | hominis | hyointestinalis |
| Campylobacter | Campylobacter | Campylobacter | Campylobacter | Campylobacter |
| iguaniorum | insulaenigrae | jejuni | lari | mucosalis |
| Campylobacter | Campylobacter | Campylobacter | Campylobacter | Campylobacter |
| ornithocola | peloridis | pinnipediorum | rectus | showae |
| Campylobacter | Campylobacter | Campylobacter | Campylobacter | Campylobacter |
| sp. 10_1_50 | sp. BCW 6879 | sp. FOBRC14 | sp. MIT 97-5078 | sp. P0023 |
| Campylobacter | Campylobacter | Campylobacter | Campylobacter | Campylobacter |
| sp. P0094 | sp. P0106 | sp. P0108 | sp. P0124 | sp. P0132 |
| Campylobacter | Campylobacter | Campylobacter | Campylobacter | Campylobacter |
| sp. P0139 | sp. P0187 | sp. P0227 | sp. P031 | sp. P090 |
| Campylobacter | Campylobacter | Campylobacter | Campylobacter | Campylobacter |
| sp. P091 | sp. P146 | sp. P148 | sp. P157 | sp. P159 |
| Campylobacter | Campylobacter | Campylobacter | Campylobacter | Campylobacter |
| sp. RM16704 | sp. RM6137 | sp. RM8835 | sp. RM8964 | sp. RM8965 |
| Campylobacter | Campylobacter | Campylobacter | Campylobacter | Campylobacter |
| sp. RM8970 | sp. S0112 | sputorum | subantarcticus | upsaliensis |
| Campylobacter | Campylobacter | Candida | Candida | Candida |
| ureolyticiis | volucris | albicans | dubliniensis | orthopsilosis |
| Candida | Candidatus | Candidatus | Candidatus | Candidatus |
| tropicalis | Accumulibacter | Amoebophilus | Aquiluna | Aquiluna |
| | phosphatis | asiaticus | sp. IMCC13023 | sp. UB-MaderosW2red |
| Candidatus | Candidatus | Candidatus | Candidatus | Candidatus |
| Arthromitus | Arthromitus | Arthromitus | Arthromitus | Atelocyanobacterium |
| sp. SFB-mouse | sp. SFB-mouse-NL | sp. SFB-rat-Yit | sp. SFB-turkey | thalassa |
| Candidatus | Candidatus | Candidatus | Candidatus | Candidatus |
| Azobacteroides | Babela | Baumannia | Berkiella | Berkiella |
| pseudotrichonymphae | massiliensis | cicadellinicola | aquae | cookevillensis |
| Candidatus | Candidatus | Candidatus | Candidatus | Candidatus |
| Blastococcus | Blochmannia | Blochmannia | Blochmannia | Brocadia |
| massiliensis | floridanus | pennsylvanicus | vafer | sapporoensis |
| Candidatus | Candidatus | Candidatus | Candidatus | Candidatus |
| Brocadia | Burkholderia | Burkholderia | Burkholderia | Carsonella |
| sinica | crenata | humilis | verschuerenii | ruddii |
| Candidatus | Candidatus | Candidatus | Candidatus | Candidatus |
| Chrysopegis | Cloacimonas | Competibacter | Contendobacter | Dactylopiibacterium |
| kryptomonas | acidaminovorans | denitrificans | odensis | carminicum |
| Candidatus | Candidatus | Candidatus | Candidatus | Candidatus |
| Desulfofervidus | Desulfonidis | Doolittlea | Dorea | Endolissoclinum |
| auxilii | audaxviator | endobia | massiliensis | faulkneri |
| Candidatus | Candidatus | Candidatus | Candidatus | Candidatus |
| Entotheonella | Erwinia | Erwinia | Evansia | Filomicrobium |
| palauensis | dacicola | sp. ErCipseudotaxifoliae | muelleri | marinum |
| Candidatus | Candidatus | Candidatus | Candidatus | Candidatus |
| Fukatsuia | Glomeribacter | Gullanella | Halobonum | Hamiltonella |
| symbiotica | gigasporarum | endobia | tyrrellensis | defensa |
| Candidatus | Candidatus | Candidatus | Candidatus | Candidatus |
| Hepatobacter | Hoaglandella | Ishikawaella | Izimaplasma | Izimaplasma |
| penaei | endobia | capsulata | sp. HR1 | sp. ZiA1 |
| Candidatus | Candidatus | Candidatus | Candidatus | Candidatus |
| Jettenia | Jidaibacter | Kinetoplastibacterium | Kinetoplastibacterium | Kinetoplastibacterium |
| caeni | acanthamoeba | crithidii | desouzaii | galatii |
| Candidatus | Candidatus | Candidatus | Candidatus | Candidatus |
| Kinetoplastibacterium | Koribacter | Kryptobacter | Kryptonium | Liberibacter |
| oncopeltii | versatilis | tengchongensis | thompsoni | africanus |
| Candidatus | Candidatus | Candidatus | Candidatus | Candidatus |
| Liberibacter | Liberibacter | Liberibacter | Magnetobacterium | Marispirochaeta |
| americanus | asiaticus | solanacearum | casensis | associata |
| Candidatus | Candidatus | Candidatus | Candidatus | Candidatus |
| Marispirochaeta | Marithrix | Methanomassiliicoccus | Methanomethylophilus | Methanomethylophilus |
| sp. JC444 | sp. Canyon 246 | intestinalis | alvus | sp. 1R26 |
| Candidatus | Candidatus | Candidatus | Candidatus | Candidatus |
| Methanoperedens | Methanoplasma | Methylopumilus | Methylopumilus | Micrarchaeota |
| nitroreducens | termitum | planktonicus | turicensis | archaeon Mia14 |
| Candidatus | Candidatus | Candidatus | Candidatus | Candidatus |
| Microthrix | Midichloria | Mikella | Moranella | Nasuia |
| parvicella | mitochondrii | endobia | endobia | deltocephalinicola |
| Candidatus | Candidatus | Candidatus | Candidatus | Candidatus |
| Neoehrlichia | Nitrosoarchaeum | Nitrosoarchaeum | Nitrosomarinus | Nitrosopelagicus |
| lotoris | koreensis | limnia | catalina | brevis |
| Candidatus | Candidatus | Candidatus | Candidatus | Candidatus |
| Nitrosopumilus | Nitrosopumilus | Nitrosotenuis | Nitrospira | Nitrospira |
| adriaticus | salaria | chungbukensis | inopinata | nitrificans |
| Candidatus | Candidatus | Candidatus | Candidatus | Candidatus |
| Nitrospira | Nucleicidtrix | Pantoea | Paraburkholderia | Paracaedibacter |
| nitrosa | amoebiphila | carbekii | schumannianae | acanthamoebae |
| Candidatus | Candidatus | Candidatus | Candidatus | Candidatus |
| Paracaedibacter | Pelagibacter | Pelagibacter | Pelagibacter | Pelagibacter |
| symbiosus | sp. HIMB1321 | sp. HTCC7211 | sp. IMCC9063 | sp. RS39 |
| Candidatus | Candidatus | Candidatus | Candidatus | Candidatus |
| Pelagibacter | Pelagibacter | Phaeomarinobacter | Photodesmus | Photodesmus |
| sp. RS40 | tibique | ectocarpi | blepharus | katoptron |
| Candidatus | Candidatus | Candidatus | Candidatus | Candidatus |
| Phytoplasma | Phytoplasma | Phytoplasma | Phytoplasma | Phytoplasma |
| australiense | mali | oryzae | phoenicium | pruni |
| Candidatus | Candidatus | Candidatus | Candidatus | Candidatus |
| Portiera | Profftella | Promineofilum | Protochlamydia | Protochlamydia |
| aleyrodidarum | armatura | breve | amoebophila | sp. W-9 |
| Candidatus | Candidatus | Candidatus | Candidatus | Candidatus |
| Puniceispirillum | Regiella | Rhodobacter | Rhodoluna | Rickettsiella |
| marinum | insecticola | lobularis | planktonica | isopodorum |
| Candidatus | Candidatus | Candidatus | Candidatus | Candidatus |
| Riesia | Riesia | Ruthia | Saccharibacteria | Schmidhempelia |
| pediculicola | pediculischaeffi | magnifica | oral taxon TM7x | bombi |
| Candidatus | Candidatus | Candidatus | Candidatus | Candidatus |
| Sodalis | Sodalis | Soleaferrea | Solibacter | Stoquefichus |
| pierantonius | sp. SoCistrobi | massiliensis | usitatus | massiliensis |
| Candidatus | Candidatus | Candidatus | Candidatus | Candidatus |
| Stoquefichus | Stoquefichus | Sulcia | Symbiobacter | Symbiothrix |
| FF sp. KLE1796 | sp. SB1 | muelleri | mobilis | dinenymphae |
| Candidatus | Candidatus | Candidatus | Candidatus | Candidatus |
| Synechococcus | Tachikawaea | Thiodiazotropha | Thiodiazotropha | Thioglobus |
| spongiarum | gelatinosa | endoloripes | endolucinida | autotrophicus |
| Candidatus | Candidatus | Candidatus | Candidatus | Candidatus |
| Thioglobus | Thiosymbion | Tremblaya | Walczuchella | Xiphinematobacter |
| singularis | oneisti | princeps | monophlebidarum | sp. Idaho Grape |
| Candidimonas | Candidimonas | Capnocytophaga | Capnocytophaga | Capnocytophaga |
| bauzanensis | nitroreducens | canimorsus | canis | cynodegmi |
| Capnocytophaga | Capnocytophaga | Capnocytophaga | Capnocytophaga | Capnocytophaga |
| gingivalis | granulosa | haemolytica | ochracea | sp. ChDC OS43 |
| Capnocytophaga | Capnocytophaga | Capnocytophaga | Capnocytophaga | Capnocytophaga |
| sp. CM59 | sp. oral taxon 323 | sp. oral taxon 324 | sp. oral taxon 326 | sp. oral taxon 329 |
| Capnocytophaga | Capnocytophaga | Capnocytophaga | Capnocytophaga | Capnocytophaga |
| sp. oral taxon 332 | sp. oral taxon 336 | sp. oral taxon 338 | sp. oral taxon 380 | sp. oral taxon 863 |
| Capnocytophaga | Capronia | Capronia | Carboxydocella | Carboxydocella |
| sputigena | coronata | epimyces | sp. JDF658 | sp. ULO1 |
| Carboxydocella | Carboxydothermus | Carboxydothermus | Carboxydothermus | Carboxydothermus |
| sporoproducens | ferrireducens | hydrogenoformans | islandicus | pertinax |
| Cardinium | Cardinium | Cardiobacterium | Cardiobacterium | Carnimonas |
| endosymbiont of | endosymbiont of | hominis | valvarum | nigrificans |
| Bemisia | Encarsia | | | |
| tabaci | pergandiella | | | |
| Carnobacterium | Carnobacterium | Carnobacterium | Carnobacterium | Carnobacterium |
| alterfunditum | divergens | funditum | gallinarum | iners |
| Carnobacterium | Carnobacterium | Carnobacterium | Carnobacterium | Carnobacterium |
| inhibens | jeotgali | maltaromaticum | mobile | pleistocenium |
| Carnobacterium | Carnobacterium | Carnobacterium | Carnobacterium | Carnobacterium |
| sp. 1290_CSPC | sp. 17-4 | sp. AT7 | sp. CP1 | sp. WN1374 |
| Carnobacterium | Carnobacterium | Caryophanon | Caryophanon | Casaltella |
| sp. ZWU0011 | viridans | latum | tenue | massiliensis |
| Castellaniella | Castellaniella | Catabacter | Catalinimonas | Catellicoccus |
| caeni | defragrans | hongkongensis | alkaloidigena | marimammalium |
| Catelliglobosispora | Catenibacterium | Catenovulum | Catenovulum | Catenulispora |
| koreensis | mitsuokai | agarivorans | maritimum | acidiphila |
| Catenuloplanes | Catonella | Caulobacter | Caulobacter | Caulobacter |
| japonicus | morbi | henricii | segnis | sp. AP07 |
| Caulobacter | Caulobacter | Caulobacter | Caulobacter | Caulobacter |
| sp. CCH4-E1 | sp. CCH5-E12 | sp. CCH9-E1 | sp. K31 | sp. OV484 |
| Caulobacter | Caulobacter | Caulobacter | Caulobacter | Caulobacter |
| sp. Root1455 | sp. Root1472 | sp. Root342 | sp. Root487D2Y | sp. Root655 |
| Caulobacter | Caulobacter | Caulobacter | Caulobacter | Caulobacter |
| sp. Root656 | sp. UNC279MFTsu5.1 | sp. UNC358MFTsu5.1 | sp. URHA0033 | vibrioides |
| Caulobacteraceae | Caulobacteraceae | Caulobacteraceae | Caviibacter | Cecembia |
| bacterium | bacterium PMMR1 | bacterium TH1-2 | abscessus | lonarensis |
| OTSz_A_272 | | | | |
| Cedecea | Cedecea | Cedecea | Celeribacter | Celeribacter |
| davisae | neteri | sp. NFIX57 | baekdonensis | ethanolicus |
| Celeribacter | Celeribacter | Celeribacter | Celeribacter | Cellulomonas |
| halophilus | indicus | marinus | neptunius | bogoriensis |
| Cellulomonas | Cellulomonas | Cellulomonas | Cellulomonas | Cellulomonas |
| carbonis | cellasea | fimi | flavigena | gilvus |
| Cellulomonas | Cellulomonas | Cellulomonas | Cellulomonas | Cellulomonas |
| iranensis | marina | massiliensis | sp. A375-1 | sp. B6 |
| Cellulomonas | Cellulomonas | Cellulomonas | Cellulomonas | Cellulomonas |
| sp. FA1 | sp. HZM | sp. KH9 | sp. KRMCY2 | sp. Leaf334 |
| Cellulomonas | Cellulomonas | Cellulomonas | Cellulomonas | Cellulomonas |
| sp. Leaf395 | sp. PSBB021 | sp. Root137 | sp. Root485 | sp. Root930 |
| Cellulomonas | Cellulomonas | Cellulomonas | Cellulophaga | Cellulophaga |
| sp. SN7 | sp. URHD0024 | sp. URHE0023 | algicola | baltica |
| Cellulophaga | Cellulophaga | Cellulophaga | Cellulophaga | Cellulophaga |
| fucicola | lytica | sp. E6(2014) | sp. Hel_1_12 | sp. W5C |
| Cellulophaga | Cellulophaga virus | Celliilosilyticum | Cellulosilyticum | Cellulosilyticum |
| tyrosinoxydans | Cba41 | lentocellum | ruminicola | sp. 115G1012 |
| Cellulosimicrobium | Cellulosimicrobium | Cellulosimicrobium | Cellulosimicrobium | Cellulosimicrobium |
| aquatile | cellulans | funkei | sp. CUA-896 | sp. I38E |
| Cellulosimicrobium sp. | Cellulosimicrobium | Cellvibrio | Cellvibrio | Cellvibrio |
| KWT-B | sp. TH-20 | japonicus | mixtus | sp. BR |
| Cellvibrio | Cellvibrio | Cellvibrio | Cellvibrio | Cellvibrionaceae |
| sp. OA-2007 | sp. pealriver | sp. PSBB006 | sp. PSBB023 | bacterium 017 |
| Centipeda | Cephaloticoccus | Cephaloticoccus | Cesiribacter | Cetobacterium |
| periodontii | capnophilus | primus | andamanensis | ceti |
| Cetobacterium | Cetobacterium | Cetobacterium | Chaetomium | Chaetomium |
| somerae | sp. ZOR0034 | sp. ZWU0022 | globosum | thermophilum |
| Chamaesiphon | Chania | Chelativorans | Chelativorans | Chelatococcus |
| minutus | multitudinisentens | sp. BNC1 | sp. J32 | daeguensis |
| Chelatococcus | Chelonobacter | Cherry necrotic rusty | Cherry virus A | Chicken anemia virus |
| sambhunathii | oris | mottle virus | | |
| Chishuiella | Chitinibacter | Chitinibacter | Chitinilyticum | Chitinilyticum |
| changwenlii | sp. ZOR0017 | tainanensis | aquatile | litopenaei |
| Chitinimonas | Chitinimonas | Chitiniphilus | Chitinivibrio | Chitinophaga |
| koreensis | taiwanensis | shinanonensis | alkaliphilus | arvensicola |
| Chitinophaga | Chitinophaga | Chitinophaga | Chitinophaga | Chitinophaga |
| costaii | eiseniae | filiformis | ginsengisegetis | jiangningensis |
| Chitinophaga | Chitinophaga | Chitinophaga | Chitinophaga | Chitinophaga |
| niabensis | pinensis | rupis | sancti | sp. CF118 |
| Chitinophaga | Chitinophaga | Chitinophaga | Chitinophagaceae | Chitinophagaceae |
| sp. YR573 | sp. YR627 | terrae | bacterium IBVUCB1 | bacterium IBVUCB2 |
| | | Kim and Jung 2007 | | |
| Chitinophagaceae | Chlamydia | Chlamydia | Chlamydia | Chlamydia |
| bacterium PMP191F | abortus | caviae | felis | gallinacea |
| Chlamydia | Chlamydia | Chlamydia | Chlamydia | Chlamydia |
| muridarum | pecorum | pneumoniae | psittaci | sp. 2742-308 |
| Chlamydia | Chlamydia | Chlamydia | Chlamydiales | Chlamydiales |
| sp. ‘Diamant’ | suis | trachomatis | bacterium | bacterium |
| | | | SCGC AB-751-O23 | SCGC AG-110-P3 |
| Chloracidobacterium | Chlorobaculum | Chlorobaculum | Chlorobaculum | Chlorobium |
| thermophilum | limnaeum | parvum | tepidum | chlorochromatii |
| Chlorobium | Chlorobium | Chlorobium | Chlorobium | Chlorobium |
| ferrooxidans | limicola | phaeobacteroides | phaeovibrioides | sp. KB01 |
| Chloroflexus | Chloroflexus | Chloroflexus | Chloroflexus | Chlorogloeopsis |
| aggregans | islandicus | sp. MS-G | sp. Y-396-1 | fritschii |
| Chloroherpeton | Chondromyces | Chondromyces | Choristoneura | Christensenella |
| thalassium | apiculatus | crocatus | fumiferana multiple | massiliensis |
| | | | nucleopolyhedrovirus | |
| Christensenella | Christensenella | Christensenella | Chromatiaceae | Chromobacterium |
| minuta | sp. AF73-05CM02 | timonensis | bacterium | amazonense |
| | | | 2141T.STBD.0c.01a | |
| Chromobacterium | Chromobacterium | Chromobacterium | Chromobacterium | Chromobacterium |
| haemolyticum | piscinae | pseudoviolaceum | sp. C-61 | sp. LK1 |
| Chromobacterium | Chromobacterium | Chromobacterium | Chromobacterium | Chromobacterium |
| sp. LK11 | sphagni | subtsugae | vaccinii | violaceum |
| Chromohalobacter | Chromohalobacter | Chromohalobacter | Chroococcidiopsis | Chroogloeocystis |
| israelensis | japonicus | salexigens | thermalis | siderophila |
| ‘Chrysanthemum | Chryseobacterium | Chryseobacterium | Chryseobacterium | Chryseobacterium |
| coronarium’ | angstadtii | antarcticum | aquaticum | arachidis |
| phytoplasma | | | | |
| Chryseobacterium | Chryseobacterium | Chryseobacterium | Chryseobacterium | Chryseobacterium |
| arthrosphaerae | artocarpi | balustinum | bovis | caeni |
| Chryseobacterium | Chryseobacterium | Chryseobacterium | Chryseobacterium | Chryseobacterium |
| carnipullorum | chaponense | contaminans | cucumeris | culicis |
| Chryseobacterium | Chryseobacterium | Chryseobacterium | Chryseobacterium | Chryseobacterium |
| daeguense | frigidisoli | gallinarum | gambrini | gleum |
| Chryseobacterium | Chryseobacterium | Chryseobacterium | Chryseobacterium | Chryseobacterium |
| greenlandense | gregarium | haifense | halperniae | hispalense |
| Chryseobacterium | Chryseobacterium | Chryseobacterium | Chryseobacterium | Chryseobacterium |
| hominis | hungaricum | indologenes | indoltheticum | jejuense |
| Chryseobacterium | Chryseobacterium | Chryseobacterium | Chryseobacterium | Chryseobacterium |
| jeonii | joostei | koreense | kwangjuense | limigenitum |
| Chryseobacterium | Chryseobacterium | Chryseobacterium | Chryseobacterium | Chryseobacterium |
| luteum | molle | oleae | oranimense | palustre |
| Chryseobacterium | Chryseobacterium | Chryseobacterium | Chryseobacterium | Chryseobacterium |
| piperi | piscicola | polytrichastri | scophthalmum | shigense |
| Chryseobacterium | Chryseobacterium | Chryseobacterium | Chryseobacterium | Chryseobacterium |
| soldanellicola | soli | solincola | sp. 6021061333 | sp. BLS98 |
| Chryseobacterium | Chryseobacterium | Chryseobacterium | Chryseobacterium | Chryseobacterium |
| sp. CBo1 | sp. CCH4-E10 | sp. CF356 | sp. ERMR1: 04 | sp. FF12 |
| Chryseobacterium | Chryseobacterium | Chryseobacterium | Chryseobacterium | Chryseobacterium |
| sp. FH1 | sp. FH2 | sp. G972 | sp. Hurlbut01 | sp. IHBB 17019 |
| Chryseobacterium | Chryseobacterium | Chryseobacterium | Chryseobacterium | Chryseobacterium |
| sp. 1HBB 10212 | sp. J200 | sp. JAH | sp. JM1 | sp. JV274 |
| Chryseobacterium | Chryseobacterium | Chryseobacterium | Chryseobacterium | Chryseobacterium |
| sp. Leaf180 | sp. Leaf201 | sp. Leaf394 | sp. Leaf404 | sp. Leaf405 |
| Chryseobacterium | Chryseobacterium | Chryseobacterium | Chryseobacterium | Chryseobacterium |
| sp. OV259 | sp. OV279 | sp. OV705 | sp. OV715 | sp. RU33C |
| Chryseobacterium | Chryseobacterium | Chryseobacterium | Chryseobacterium | Chryseobacterium |
| sp. RU37D | sp. StRB126 | sp. T16E-39 | sp. UNC8MFCol | sp. VAUSW3 |
| Chryseobacterium | Chryseobacterium | Chryseobacterium | Chryseobacterium | Chryseobacterium |
| sp. VT16-26 | sp. YR005 | sp. YR203 | sp. YR221 | sp. YR459 |
| Chryseobacterium | Chryseobacterium | Chryseobacterium | Chryseobacterium | Chryseobacterium |
| sp. YR460 | sp. YR477 | sp. YR480 | sp. YR485 | sp. YR561 |
| Chryseobacterium | Chryseobacterium | Chryseobacterium | Chryseobacterium | Chryseobacterium |
| taeanense | taichungense | taihuense | taiwanense | takakiae |
| Chryseobacterium | Chryseobacterium | Chryseobacterium | Chryseobacterium | Chryseobacterium |
| taklimakanense | tenax | treverense | ureilyticum | vrystaatense |
| Chryseobacterium | Chryseobacterium | Chryseolinea | Chrysiogenes | Chthoniobacter |
| wanjuense | zeae | serpens | arsenatis | flavus |
| Chthonomonas | Citreicella | Citreicella | Citreicella | Citreicella |
| calidirosea | marina | sp. 357 | sp. SE45 | thiooxidans |
| Citreimonas | Citricoccus | Citrobacter | Citrobacter | Citrobacter |
| salinaria | sp. CH26A | amalonaticus | braakii | europaeus |
| Citrobacter | Citrobacter | Citrobacter | Citrobacter | Citrobacter |
| farmeri | freundii | koseri | pasteurii | portucalensis |
| Citrobacter | Citrobacter | Citrobacter | Citrobacter | Citrobacter |
| rodentium | sedlakii | sp. 30_2 | sp. 50677481 | sp. A316 |
| Citrobacter | Citrobacter | Citrobacter | Citrobacter | Citrobacter |
| sp. AATXQ | sp. AATXR | sp. BIDMC107 | sp. BIDMC108 | sp. CFSAN044567 |
| Citrobacter | Citrobacter | Citrobacter | Citrobacter | Citrobacter |
| sp. CtB7.12 | sp. FDAARGOS_156 | sp. JT3 | sp. KTE151 | sp. KTE30 |
| Citrobacter | Citrobacter | Citrobacter | Citrobacter | Citrobacter |
| sp. MGH 55 | sp. MGH100 | sp. MGH103 | sp. MGH104 | sp. MGH105 |
| Citrobacter | Citrobacter | Citrobacter | Citrobacter | Citrobacter |
| sp. MGH106 | sp. MGH109 | sp. MGH110 | sp. MGH99 | sp. NLAE-zl-C269 |
| Citrobacter | Citrobacter | Citrobacter | Citromicrobium | Citromicrobium |
| sp. S-77 | werkmanii | youngae | sp. JLT1363 | sp. WPS32 |
| Cladophialophora | Cladophialophora | Cladophialophora | Cladophialophora | Cladophialophora |
| bantiana | carrionii | immunda | psammophila | yegresii |
| Clavibacter cf. | Clavibacter | Clavispora | Cloacibacillus | Cloacibacillus |
| michiganensis LMG | michiganensis | lusitaniae | evryensis | porcorum |
| 26808 | | | | |
| Cloacibacillus | Cloacibacterium | Clostridia | Clostridia | Clostridia |
| sp. An23 | normanense | bacterium | bacterium | bacterium |
| | | UC5.1-1C12 | UC5.1-1D1 | UC5.1-1D10 |
| Clostridia | Clostridia | Clostridia | Clostridia | Clostridiaceae |
| bacterium | bacterium | bacterium | bacterium | bacterium MS3 |
| UC5.1-1E11 | UC5.1-2F7 | UC5.1-2H11 | UC5.1-2H6 | |
| Clostridiales | Clostridiales | Clostridiales | Clostridiales | Clostridiales |
| bacterium | bacterium 1_7_47FAA | bacterium CHKC1001 | bacterium CHKC1006 | bacterium DRI-13 |
| Clostridiales | Clostridiales | Clostridiales | Clostridiales | Clostridiales |
| bacterium KA00134 | bacterium KA00274 | bacterium KLE1615 | bacterium | bacterium MCWD3 |
| | | | Marseille-P2986 | |
| Clostridiales | Clostridiales | Clostridiales | Clostridiales | Clostridiales |
| bacterium mt11 | bacterium NK3B98 | bacterium | bacterium S5-A14a | bacterium SIT11 |
| | | oral taxon 876 | | |
| Clostridiales | Clostridiales | Clostridiales | Clostridiales | Clostridiales |
| bacterium SK-Y3 | bacterium VE202-01 | bacterium VE202-03 | bacterium VE202-06 | bacterium VE202-08 |
| Clostridiales | Clostridiales | Clostridiales | Clostridiales | Clostridiales |
| bacterium VE202-09 | bacterium VE202-13 | bacterium VE202-14 | bacterium VE202-15 | bacterium VE202-16 |
| Clostridiales | Clostridiales | Clostridiales | Clostridiales | Clostridiales |
| bacterium VE202-18 | bacterium VE202-21 | bacterium VE202-26 | bacterium VE202-27 | bacterium VE202-28 |
| Clostridiales | Clostridiisalibacter | Clostridioides | Clostridioides | Clostridium |
| bacterium VE202-29 | paucivorans | difficile | mangenotii | aceticum |
| Clostridium | Clostridium | Clostridium | Clostridium | Clostridium |
| acetireducens | acetobutylicum | acidisoli | akagii | algidicarnis |
| Clostridium | Clostridium | Clostridium | Clostridium | Clostridium |
| amylolyticum | arbusti | argentinense | aurantibutyricum | autoethanogenum |
| Clostridium | Clostridium | Clostridium | Clostridium | Clostridium |
| baratii | beijerinckii | bornimense | botulinum | butyricum |
| Clostridium | Clostridium | Clostridium | Clostridium | Clostridium |
| cadaveris | carboxidivorans | cavendishii | celatum | cellulovorans |
| Clostridium | Clostridium | Clostridium | Clostridium | Clostridium |
| chauvoei | chromiireducens | cochlearium | colicanis | collagenovorans |
| Clostridium | Clostridium | Clostridium | Clostridium | Clostridium |
| coskatii | cylindrosporum | diolis | disporicum | drakei |
| Clostridium | Clostridium | Clostridium | Clostridium | Clostridium |
| estertheticum | fallax | felsineum | formicaceticum | frigidicarnis |
| Clostridium | Clostridium | Clostridium | Clostridium | Clostridium |
| gasigenes | grantii | haemolyticum | homopropionicum | hydrogeniformans |
| Clostridium | Clostridium | Clostridium | Clostridium | Clostridium |
| ihumii | intestinale | jeddahense | kluyveri | ljungdahlii |
| Clostridium | Clostridium | Clostridium | Clostridium | Clostridium |
| lundense | magnum | massiliodielmoense | merdae | neonatale |
| Clostridium | Clostridium | Clostridium | Clostridium | Clostridium |
| novyi | oryzae | paraputrificum | pasteurianum | perfringens |
| Clostridium | Clostridium | Clostridium | Clostridium | Clostridium |
| phage c-st | phage phi24R | phoceensis | polynesiense | puniceum |
| Clostridium | Clostridium | Clostridium | Clostridium | Clostridium |
| ragsdalei | roseum | saccharobutylicum | saccharoperbutylacetonicum | sartagoforme |
| Clostridium | Clostridium | Clostridium | Clostridium | Clostridium |
| saudiense | scatologenes | senegalense | septicum | sp. 1_1_41A1FAA |
| Clostridium | Clostridium | Clostridium | Clostridium | Clostridium |
| sp. 12(A) | sp. 7_2_43FAA | sp. 7_3_54FAA | sp. Ade. TY | sp. ASBs410 |
| Clostridium | Clostridium | Clostridium | Clostridium | Clostridium |
| sp. ASF356 | sp. ASF502 | sp. AT4 | sp. ATCC 25772 | sp. ATCC 29733 |
| Clostridium | Clostridium | Clostridium | Clostridium | Clostridium |
| sp. ATCC BAA-442 | sp. Bc-iso-3 | sp. BL8 | sp. BL-8 | sp. BNL1100 |
| Clostridium | Clostridium | Clostridium | Clostridium | Clostridium |
| sp. C105KSO13 | sp. C105KSO14 | sp. C105KSO15 | sp. C8 | sp. CL-2 |
| Clostridium | Clostridium | Clostridium | Clostridium | Clostridium |
| sp. CL-6 | sp. D5 | sp. DL-VIII | sp. DMHC 10 | sp. DSM |
| | | | | 18688 |
| Clostridium | Clostridium | Clostridium | Clostridium | Clostridium |
| sp. DSM 4029 | sp. DSM 8431 | sp. FS41 | sp. HGF2 | sp. HMSC19A10 |
| Clostridium | Clostridium | Clostridium | Clostridium | Clostridium |
| sp. HMSC19A11 | sp. HMSC19B01 | sp. HMSC19B10 | sp. HMSC19B11 | sp. HMSC19C05 |
| Clostridium | Clostridium | Clostridium | Clostridium | Clostridium |
| sp. HMSC19C08 | sp. HMSC19D02 | sp. HMSC19D07 | sp. IBU/N125C | sp. IBUN62F |
| Clostridium | Clostridium | Clostridium | Clostridium | Clostridium |
| sp. K25 | sp. KLE 1755 | sp. KNHs205 | sp. KNHs209 | sp. KNHs214 |
| Clostridium | Clostridium | Clostridium | Clostridium | Clostridium |
| sp. L2-50 | sp. L74 | sp. LF2 | sp. M62/1 | sp. Marseille-P2414 |
| Clostridium | Clostridium | Clostridium | Clostridium | Clostridium |
| sp. Marseille-P2415 | sp. Marseille-P2434 | sp. Marseille-P2538 | sp. Marseille-P299 | sp. Marseille-P3244 |
| Clostridium | Clostridium | Clostridium | Clostridium | Clostridium |
| sp. MSTE9 | sp. mt5 | sp. N3C | sp. NCR | sp. ND2 |
| Clostridium | Clostridium | Clostridium | Clostridium | Clostridium |
| sp. SS2/1 | sp. SY8519 | sp. USBA 49 | sp. W14A | sporogenes |
| Clostridium | Clostridium | Clostridium | Clostridium | Clostridium |
| sulfidigenes | taeniosporum | tepidiprofundi | tepidum | tetani |
| Clostridium | Clostridium | Clostridium | Clostridium | Clostridium |
| tetanomorphum | thermobutyricum | tunisiense | tyrobutyricum | uliginosum |
| Clostridium | Cnuella | Cnuibacter | Cobetia | Cobetia |
| ventriculi | takakiae | physcomitrellae | amphilecti | crustatorum |
| Cobetia | Cobetia | Cobetia | Cobetia | Coccidioides |
| marina | sp. MM11DA2H-1 | sp. QF-1 | sp. UCD-24C | immitis |
| Coccidioides | Cohaesibacter | Cohnella | Cohnella | Cohnella |
| posadasii | marisflavi | kolymensis | laeviribosi | panacarvi |
| Cohnella | Cohnella | Cohnella | Cohnella | Coleofasciculus |
| sp. 6021052837 | sp. CIP 111063 | sp. 0V330 | thermotolerans | chthonoplastes |
| Colibacter | Colletotrichum | Colletotrichum | Colletotrichum | Colletotrichum |
| massiliensis | fioriniae | gloeosporioides | graminicola | higginsianum |
| Collimonas | Collimonas | Collimonas | Collimonas | Collimonas |
| arenae | fungivorous | pratensis | sp. OK242 | sp. OK307 |
| Collimonas | Collimonas | Collinsella | Collinsella | Collinsella |
| sp. OK412 | sp. OK607 | aerofaciens | bouchesdurhonensis | ihuae |
| Collinsella | Collinsella | Collinsella | Collinsella | Collinsella |
| intestinalis | phocaeensis | sp. 4_8_47FAA | sp. An2 | sp. An268 |
| Collinsella | Collinsella | Collinsella | Collinsella | Collinsella |
| sp. An271 | sp. An307 | sp. An7 | sp. Marseille-P2666 | sp. MS5 |
| Collinsella | Collinsella | Collinsella | Colwellia | Colwellia |
| sp. TF06-26 | stercoris | tanakaei | aestuarii | agarivorans |
| Colwellia | Colwellia | Colwellia | Colwellia | Colwellia |
| chukchiensis | marinimaniae | mytili | piezophila | polaris |
| Colwellia | Colwellia | Colwellia | Colwellia | Colwellia |
| psychrerythraea | sediminilitoris | sp. NB097-1 | sp. PAMC 20917 | sp. PAMC 21821 |
| Colwellia | Colwellia | Comamonadaceae | Comamonadaceae | Comamonadaceae |
| sp. TT2012 | sp. UCD-KL20 | bacterium Al | bacterium B1 | bacterium CCH12-A10 |
| Comamonadaceae | Comamonadaceae | Comamonadaceae | Comamonadaceae | Comamonadaceae |
| bacterium CCH4-C5 | bacterium H1 | bacterium NML00-0135 | bacterium NML03-0146 | bacterium NML120219 |
| Comamonadaceae | Comamonadaceae | Comamonadaceae | Comamonas | Comamonas |
| bacterium NML79-0751 | bacterium NML97-0112 | bacterium URHA0028 | aquatica | badia |
| Comamonas | Comamonas | Comamonas | Comamonas | Comamonas |
| composti | granuli | kerstersii | serinivorans | sp. B-9 |
| Comamonas | Comamonas | Comamonas | Comamonas | Comamonas |
| sp. E6 | terrae | terrigena | testosteroni | thiooxydans |
| Commensalibacter | Commensalibacter | Conchiformibius | Conchiformibius | Conexibacter |
| intestini | sp. MX01 | kuhniae | steedae | woesei |
| Confluentimicrobium | Confluentimicrobium | Congregibacter | Coniophora | Coniosporium |
| lipolyticum | sp. EMB200-NS6 | litoralis | puteana | apollinis |
| Coprinopsis | Coprobacillus | Coprobacillus | Coprobacillus | Coprobacillus |
| cinerea | sp. 29_1 | sp. 3_3_56FAA | sp. 8_1_38FAA | sp. 8_2_54BFAA |
| Coprobacillus | Coprobacillus | Coprobacter | Coprobacter | Coprococcus |
| sp. D6 | sp. D7 | fastidiosus | secundus | comes |
| Coprococcus | Coprococcus | Coprococcus | Coprothermobacter | Coprothermobacter |
| eutactus | sp. HPP0048 | sp. HPP0074 | platensis | proteolyticus |
| Coraliomargarita | Corallococcus | Cordyceps | Coriobacteriaceae | Coriobacteriaceae |
| akajimensis | coralloides | militaris | bacterium 68-1-3 | bacterium B V3Ac1 |
| Coriobacteriaceae | Coriobacteriales | Coriobacterium | Corynebacterium | Corynebacterium |
| bacterium CHKC1002 | bacterium DNF00809 | glomerans | accolens | afermentans |
| Corynebacterium | Corynebacterium | Corynebacterium | Corynebacterium | Corynebacterium |
| ammoniagenes | amycolatum | appendicis | aquilae | argentoratense |
| Corynebacterium | Corynebacterium | Corynebacterium | Corynebacterium | Corynebacterium |
| atypicum | aurimucosum | auriscanis | bouchesdurhonense | bovis |
| Corynebacterium | Corynebacterium | Corynebacterium | Corynebacterium | Corynebacterium |
| callunae | camporealensis | capitovis | casei | caspium |
| Corynebacterium | Corynebacterium | Corynebacterium | Corynebacterium | Corynebacterium |
| ciconiae | coyleae | crudilactis | cystitidis | deserti |
| Corynebacterium | Corynebacterium | Corynebacterium | Corynebacterium | Corynebacterium |
| diphtheriae | doosanense | durum | efficiens | epidermidicanis |
| Corynebacterium | Corynebacterium | Corynebacterium | Corynebacterium | Corynebacterium |
| falsenii | flavescens | fournierii | frankenforstense | freiburgense |
| Corynebacterium | Corynebacterium | Corynebacterium | Corynebacterium | Corynebacterium |
| freneyi | genitalium | glucuronolyticum | glutamicum | glyciniphilum |
| Corynebacterium | Corynebacterium | Corynebacterium | Corynebacterium | Corynebacterium |
| halotolerans | humireducens | ihumii | imitans | jeddahense |
| Corynebacterium | Corynebacterium | Corynebacterium | Corynebacterium | Corynebacterium |
| jeikeium | kroppenstedtii | kutscheri | lactis | lipophiloflavum |
| Corynebacterium | Corynebacterium | Corynebacterium | Corynebacterium | Corynebacterium |
| lowii | lubricantis | marinum | maris | massiliense |
| Corynebacterium | Corynebacterium | Corynebacterium | Corynebacterium | Corynebacterium |
| mastitidis | matmchotii | minutissimum | mustelae | mycetoides |
| Corynebacterium | Corynebacterium | Corynebacterium | Corynebacterium | Corynebacterium |
| nuruki | oculi | phocae | pilosum | pollutisoli |
| Corynebacterium | Corynebacterium | Corynebacterium | Corynebacterium | Corynebacterium |
| propinquum | provencense | pseudodiphtheriticum | pseudogenitalium | pseudotuberculosis |
| Corynebacterium | Corynebacterium | Corynebacterium | Corynebacterium | Corynebacterium |
| pyruviciproducens | renale | resistens | riegelii | simulans |
| Corynebacterium | Corynebacterium | Corynebacterium | Corynebacterium | Corynebacterium |
| singulare | sp. CMW7794 | sp. CNJ-954 | sp. EPI-003-04- | sp. HMSC034A01 |
| | | | 2554_SCH2473622 | |
| Corynebacterium | Corynebacterium | Corynebacterium | Corynebacterium | Corynebacterium |
| sp. HMSC034B08 | sp. HMSC034E11 | sp. HMSC035E02 | sp. HMSC036D03 | sp. HMSC036E10 |
| Corynebacterium | Corynebacterium | Corynebacterium | Corynebacterium | Corynebacterium |
| sp. HMSC04H06 | sp. HMSC058E07 | sp. HMSC05C01 | sp. HMSC05D03 | sp. HMSC05E07 |
| Corynebacterium | Corynebacterium | Corynebacterium | Corynebacterium | Corynebacterium |
| sp. HMSC05H05 | sp. HMSC062A03 | sp. HMSC065D07 | sp. HMSC067D03 | sp. HMSC069E04 |
| Corynebacterium | Corynebacterium | Corynebacterium | Corynebacterium | Corynebacterium |
| sp. HMSC06C06 | sp. HMSC070E08 | sp. HMSC071B10 | sp. HMSC071F07 | sp. HMSC072A02 |
| Corynebacterium | Corynebacterium | Corynebacterium | Corynebacterium | Corynebacterium |
| sp. HMSC072D01 | sp. HMSC072D12 | sp. HMSC073D01 | sp. HMSC073H12 | sp. HMSC074A01 |
| Corynebacterium | Corynebacterium | Corynebacterium | Corynebacterium | Corynebacterium |
| sp. HMSC074C03 | sp. HMSC074C11 | sp. HMSC074E01 | sp. HMSC076C10 | sp. HMSC077D03 |
| Corynebacterium | Corynebacterium | Corynebacterium | Corynebacterium | Corynebacterium |
| sp. HMSC078A10 | sp. HMSC078H07 | sp. HMSC08A12 | sp. HMSC08D02 | sp. HMSC08F01 |
| Corynebacterium | Corynebacterium | Corynebacterium | Corynebacterium | Corynebacterium |
| sp. HMSC11D10 | sp. HMSC11E11 | sp. HMSC11H10 | sp. HMSC22B11 | sp. HMSC27B11 |
| Corynebacterium | Corynebacterium | Corynebacterium | Corynebacterium | Corynebacterium |
| sp. HMSC28B08 | sp. HMSC29G08 | sp. HMSC30G07 | sp. KPL1818 | sp. KPL1824 |
| Corynebacterium | Corynebacterium | Corynebacterium | Corynebacterium | Corynebacterium |
| sp. Marseille-P2417 | sp. MC3 | sp. NBT06-6 | sp. NML 120412 | sp. NML 150383 |
| Corynebacterium | Corynebacterium | Corynebacterium | Corynebacterium | Corynebacterium |
| sp. NML00-0156 | sp. NML120713 | sp. NML130628 | sp. NML140438 | sp. NML92-0415 |
| Corynebacterium | Corynebacterium | Corynebacterium | Corynebacterium | Corynebacterium |
| sp. NML93-0607 | sp. NML98-0116 | sp. NML99-0020 | sp. OG2 | sphenisci |
| Corynebacterium | Corynebacterium | Corynebacterium | Corynebacterium | Corynebacterium |
| spheniscorum | sputi | stationis | striatum | terpenotabidum |
| Corynebacterium | Corynebacterium | Corynebacterium | Corynebacterium | Corynebacterium |
| testudinoris | timonense | tuberculostearicum | tuscaniense | ulcerans |
| Corynebacterium | Corynebacterium | Corynebacterium | Corynebacterium | Corynebacterium |
| ulceribovis | urealyticum | ureicelerivorans | tirinapleomorphum | utere/jui |
| Corynebacterium | Corynebacterium | Corynebacterium | Corynebacterium-like | Cosenzaea |
| variabile | vitaeruminis | xerosis | bacterium B27 | myxofaciens |
| Couchioplanes | Coxiella | Coxiella endosymbiont | Coxiella-like | Crenobacter |
| caeruleus | burnetii | of Amblyomma | endosymbiont | luteus |
| | | americanum | | |
| Crenotalea | Crenothrix | Criblamydia | Cribrihabitans | Criibacterium |
| thermophila | polyspora | sequanensis | marinus | bergeronii |
| Crinalium | Croceibacter | Croceicoccus | Croceicoccus | Croceicoccus |
| epipsammum | atlanticus | marinus | mobilis | naphthovorans |
| Croceicoccus | Croceicoccus | Croceitalea | Crocinitomix | Crocinitomix |
| pelagius | sp. H4 | dokdonensis | catalasitica | sp. 0182 |
| Crocosphaera | Cronobacter | Cronobacter | Cronobacter | Cronobacter |
| watsonii | condimenti | dublinensis | malonaticus | muytjensii |
| Cronobacter | Cronobacter | Cronobacter | Crossiella | Cruoricaptor |
| sakazakii | turicensis | universalis | equi | ignavus |
| Cryobacterium | Cryobacterium | Cryobacterium | Cryobacterium | Cryobacterium |
| arcticum | flavum | levicorallinum | luteum | psychrotolerans |
| Cryobacterium | Cryobacterium | Cryobacterium | Cryocola | Cryptobacterium |
| roopkundense | sp. LW097 | sp. MLB-32 | sp. 340MFSha3.1 | curtum |
| Cryptococcus | Cryptococcus | Cryptosporangium | Cryptosporangium | Cryptosporidium |
| amylolentus | neoformans | arvum | aurantiacum | hominis |
| Cryptosporidium | Cryptosporidium | Cucumibacter | Culturomica | Cuniculiplasma |
| muris | parvum | marinus | massiliensis | divulgatum |
| Cupriavidus | Cupriavidus | Cupriavidus | Cupriavidus | Cupriavidus |
| alkaliphilus | basilensis | gilardii | metallidurans | nantongensis |
| Cupriavidus | Cupriavidus | Cupriavidus | Cupriavidus | Cupriavidus |
| necator | oxalaticus | pauculus | pinatubonensis | sp. amp6 |
| Cupriavidus | Cupriavidus | Cupriavidus | Cupriavidus | Cupriavidus |
| sp. BIS7 | sp. D384 | sp. HMR-1 | sp. HPC(L) | sp. IDO |
| Cupriavidus | Cupriavidus | Cupriavidus | Cupriavidus | Cupriavidus |
| sp. SK-3 | sp. SK-4 | sp. USMAA1020 | sp. USMAA2-4 | sp. USMAHM13 |
| Cupriavidus | Cupriavidus | Cupriavidus | Cupriavidus | Curtobacterium |
| sp. UYPR2.512 | sp. WS | sp. YR651 | taiwanensis | ammoniigenes |
| Curtobacterium | Curtobacterium | Curtobacterium | Curtobacterium | Curtobacterium |
| citreum | flaccumfaciens | luteum | oceanosedimentum | pusillum |
| Curtobacterium | Curtobacterium | Curtobacterium | Curtobacterium | Curtobacterium |
| sp. B18 | sp. B8 | sp. BH-2-1-1 | sp. ER1/6 | sp. Leaf183 |
| Curtobacterium | Curtobacterium | Curtobacterium | Curtobacterium | Curtobacterium |
| sp. Leaf261 | sp. MCBA15_001 | sp. MCBA15_004 | sp. MCBA15_005 | sp. MCBA15_008 |
| Curtobacterium | Curtobacterium | Curtobacterium | Curtobacterium | Curtobacterium |
| sp. MCBA15_009 | sp. MCBA15_012 | sp. MCBA15_013 | sp. MMLR14_010 | sp. MR_MD2014 |
| Curtobacterium | Curtobacterium | Curtobacterium | Curtobacterium | Curvibacter |
| sp. S6 | sp. UCD-KPL2560 | sp. UNCCL17 | sp. UNCCL20 | delicatus |
| Curvibacter | Curvibacter | Curvibacter | Curvibacter | Cutaneotrichosporon |
| gracilis | lanceolatus | sp. AEP1-3 | sp. PAE-UM | oleaginosus |
| Cutibacterium | Cutibacterium | Cutibacterium | Cyanobacterium | cyanobacterium |
| acnes | avidum | granulosum | aponinum | endosymbiont of |
| | | | | Epithemia turgida |
| cyanobacterium | Cyanobacterium | Cyanobium | Cyanobium | Cyanobium |
| PCC 7702 | sp. IPPAS B-1200 | gracile | sp. NIES-981 | sp. PCC 7001 |
| Cyanophage S-TIM5 | Cyanothece | Cyanothece | Cyanothece | Cyanothece |
| | sp. CCY0110 | sp. PCC 7424 | sp. PCC 7425 | sp. PCC 7822 |
| Cyanothece | Cyanothece | Cyberlindnera | Cyclobacterium | Cyclobacterium |
| sp. PCC 8801 | sp. PCC 8802 | jadinii | amurskyense | halophilum |
| Cyclobacterium | Cyclobacterium | Cyclobacterium | Cylindrospermopsis | Cylindrospermum |
| lianum | marinum | qasimii | raciborskii | stagnale |
| Cyphellophora | Cystobacter | Cystobacter | Cytophaga | Cytophaga |
| europaea | ferrugineus | fuscus | aurantiaca | hutchinsonii |
| Cytophagales | Dactylellina | Dactylococcopsis | Dactylosporangium | Dakarella |
| bacterium B6 | haptotyla | salina | aurantiacum | massiliensis |
| Dasania | Debaryomyces | Debaryomyces | Dechloromonas | Dechloromonas |
| marina | fabryi | hansenii | agitata | aromatica |
| Dechloromonas | Deefgea | Deferribacter | Deferrisoma | Defluviimonas |
| denitrificans | rivuli | desulfuricans | camini | alba |
| Defluviitalea | Defluviitoga | Dehalobacter | Dehalobacter | Dehalobacter |
| phaphyphila | tunisiensis | restrictus | sp. CF | sp. DCA |
| Dehalobacter | Dehalobacter | Dehalobacter | Dehalobacter | Dehalobacterium |
| sp. E1 | sp. FTH1 | sp. TeCB1 | sp. UNSWDHB | formicoaceticum |
| Dehalococcoides | Dehalococcoides | Dehalogenimonas | Dehalogenimonas | Dehalogenimonas |
| mccartyi | sp. UCH007 | alkenigignens | formicexedens | lykanthroporepellens |
| Deinococcus | Deinococcus | Deinococcus | Deinococcus | Deinococcus |
| actinosclerus | apachensis | aquatilis | deserti | ficus |
| Deinococcus | Deinococcus | Deinococcus | Deinococcus | Deinococcus |
| frigens | geothermalis | gobiensis | grandis | hopiensis |
| Deinococcus | Deinococcus | Deinococcus | Deinococcus | Deinococcus |
| indicus | maricopensis | marmoris | misasensis | murrayi |
| Deinococcus | Deinococcus | Deinococcus | Deinococcus | Deinococcus |
| peraridilitoris | phoenicis | pimensis | proteolyticus | puniceus |
| Deinococcus | Deinococcus | Deinococcus | Deinococcus | Deinococcus |
| radiodurans | reticulitermitis | soli Cha | sp. 2009 | sp. Leaf326 |
| | | et al. 2016 | | |
| Deinococcus | Deinococcus | Deinococcus | Deinococcus | Deinococcus |
| sp. LM3 | sp. RL | sp. YIM 77859 | swuensis | wulumuqiensis |
| Delftia | Delftia | Delftia | Delftia | Delftia |
| acidovorans | lacustris | sp. 670 | sp. Cs1-4 | sp. GW456-R20 |
| Delftia | Delftia | Delftia | Delftia | Delftia |
| sp. JD2 | sp. K82 | sp. RIT313 | sp. ZNC0008 | tsuruhatensis |
| delta proteobacterium | delta proteobacterium | delta proteobacterium | delta proteobacterium | delta proteobacterium |
| MLMS-1 | NaphS2 | PSCGC 5296 | PSCGC 5342 | PSCGC 5451 |
| Demequina | Demequina | Demequina | Demequina | Demequina |
| aestuarii | aurantiaca | flava | globuliformis | lutea |
| Demequina | Demequina | Demequina | Demequina | Demequina |
| oxidasica | salsinemoris | sediminicola | sp. NBRC | sp. NBRC |
| | | | 110051 | 110052 |
| Demequina | Demequina | Demequina | Demequina | Demequina |
| sp. NBRC | sp. NBRC | sp. NBRC | sp. NBRC | sp. NBRC |
| 110053 | 110054 | 110055 | 110056 | 110057 |
| Demetria | Dendrosporobacter | Denitrobacterium | Denitrovibrio | Dermabacter |
| terragena | quercicolus | detoxificans | acetiphilus | hominis |
| Dermabacter | Dermabacter | Dermabacter | Dermabacter | Dermacoccus |
| sp. HFH0086 | sp. HMSC06F07 | sp. HMSC08H10 | vaginalis | nishinomiyaensis |
| Dermacoccus | Dermacoccus | Dermatophilus | Derxia | Derxia |
| sp. Ellin185 | sp. PE3 | congolensis | gummosa | lacustris |
| Desemzia | Desertifilum | Desmospora | Desnuesiella | Desulfacinum |
| incerta | sp. IPPAS B-1220 | sp. 8437 | massiliensis | hydrothermale |
| Desulfacinum | Desulfamplus | Desulfarculus | Desiilfatibacillum | Desulfatibacillum |
| infernum | magnetovallimortis | baarsii | aliphaticivorans | alkenivorans |
| Desulfatiglans | Desulfatirhabdium | Desulfatitalea | Desulfitibacter | Desulfitobacterium |
| anilini | butyrativorans | tepidiphila | alkalitolerans | chlororespirans |
| Desulfitobacterium | Desulfitobacterium | Desulfitobacterium | Desulfitobacterium | Desulfitobacterium |
| dehalogenans | dichloroeliminans | hafniense | metallireducens | sp. PCE1 |
| Desulfobacca | Desulfobacter | Desulfobacter | Desulfobacter | Desulfobacterium |
| acetoxidans | curvatus | postgatei | vibrioformis | autotrophicum |
| Desulfobacterium | Desulfobacula | Desulfobacula | Desulfobacula | Desulfobulbus |
| vacuolatum | phenolica | sp. TS | toluolica | elongatus |
| Desulfobulbus | Desulfobulbus | Desulfobulbus | Desulfocapsa | Desulfocarbo |
| japonicus | mediterraneus | propionicus | sulfexigens | indianensis |
| Desulfococcus | Desulfococcus | Desulfocurvus | Desulfofustis | Desulfohalobium |
| multivorans | oleovorans | vexinensis | glycolicus | retbaense |
| Desulfoluna | Desulfomicrobium | Desulfomicrobium | Desulfomicrobium | Desulfomicrobium |
| spongiiphila | apsheronum | baculatum | escambiense | norvegicum |
| Desulfomicrobium | Desulfomonile | Desulfonatronospira | Desulfonatronovibrio | Desulfonatronovibrio |
| orale | tiedjei | thiodismutans | hydrogenovorans | magnus |
| Desulfonatronum | Desulfonatronum | Desulfonatronum | Desulfonatronum | Desulfonauticus |
| lacustre | thioautotrophicum | thiodismutans | thiosulfatophilum | submarinus |
| Desulfonispora | Desulfopila | Desulfoplanes | Desulforegula | Desulforhopalus |
| thiosulfatigenes | aestuarii | formicivorans | conservatrix | singaporensis |
| Desulfosarcina | Desulfosarcina | Desulfospira | Desulfosporosinus | Desulfosporosinus |
| cetonica | sp. BuS5 | joergensenii | acididurans | acidiphilus |
| Desulfosporosinus | Desulfosporosinus | Desulfosporosinus | Desulfosporosinus | Desulfosporosinus |
| hippei | lacus | meridiei | orientis | sp. BG |
| Desulfosporosinus | Desulfosporosinus | Desulfosporosinus | Desulfosporosinus | Desulfosporosinus |
| sp. FKA | sp. FKB | sp. HMP52 | sp. I2 | sp. OL |
| Desulfosporosinus | Desulfosporosinus | Desulfotalea | Desulfotignum | Desulfotignum |
| sp. OF | youngiae | psychrophila | balticum | phosphitoxidans |
| Desulfotomaculum | Desulfotomaculum | Desulfotomaculum | Desulfotomaculum | Desulfotomaculum |
| acetoxidans | aeronauticum | alcoholivorax | alkaliphilum | arcticum |
| Desulfotomaculum | Desulfotomaculum | Desulfotomaculum | Desulfotomaculum | Desulfotomaculum |
| australicum | copahuensis | ferrireducens | geothermicum | gibsoniae |
| Desulfotomaculum | Desulfotomaculum | Desulfotomaculum | Desulfotomaculum | Desulfotomaculum |
| hydrothermale | intricatum | nigrificans | putei | reducens |
| Desulfotomaculum | Desulfotomaculum | Desulfotomaculum | Desulfovermiculus | Desulfovibrio |
| ruminis | thermocisternum | thermosubterraneum | halophilus | aespoeensis |
| Desulfovibrio | Desulfovibrio | Desulfovibrio | Desulfovibrio | Desulfovibrio |
| africanus | alaskensis | alcoholivorans | alkalitolerans | aminophilus |
| Desulfovibrio | Desulfovibrio | Desulfovibrio | Desulfovibrio | Desulfovibrio |
| bastinii | bizertensis | brasiliensis | cuneatus | dechloracetivorans |
| Desulfovibrio | Desulfovibrio | Desulfovibrio | Desulfovibrio | Desulfovibrio |
| desulfuricans | fairfieldensis | ferrireducens | frigidus | fructosivorans |
| Desulfovibrio | Desulfovibrio | Desulfovibrio | Desulfovibrio | Desulfovibrio |
| gigas | gracilis | hydrothermalis | indicus | inopinatus |
| Desulfovibrio | Desulfovibrio | Desulfovibrio | Desulfovibrio | Desulfovibrio |
| legallii | litoralis | longus | magneticus | mexicanus |
| Desulfovibrio | Desulfovibrio | Desulfovibrio | Desulfovibrio | Desulfovibrio |
| oxyclinae | piezophilus | piger | putealis | salexigens |
| Desulfovibrio | Desulfovibrio | Desulfovibrio | Desulfovibrio | Desulfovibrio |
| sp. 3_1_syn3 | sp. 6_1_46AFAA | sp. A2 | sp. An276 | sp. DV |
| Desulfovibrio | Desulfovibrio | Desulfovibrio | Desulfovibrio | Desulfovibrio |
| sp. FW1012B | sp. K3S | sp. L21-Syr-AB | sp. TomC | sp. U5L |
| Desulfovibrio | Desulfovibrio | Desulfovibrio | Desulfovibrio | Desulfovirgula |
| sp. X2 | termitidis | vulgaris | zosterae | thermocuniculi |
| Desulfurella | Desulfurella | Desulfuribacillus | Desulfuribacillus | Desulfurispirillum |
| amilsii | multipotens | alkaliarsenatis | stibiiarsenatis | indicum |
| Desulfurispora | Desulfurivibrio | Desulfurobacterium | Desulfurobacterium | Desulfurobacterium |
| thermophila | alkaliphilus | atlanticum | indicum | sp. TC5-1 |
| Desulfurobacterium | Desulfuromonas | Desulfuromonas | Desulfuromonas | Desulfuromonas |
| thermolithotrophum | acetexigens | acetoxidans | soudanensis | sp. DDH964 |
| Desulfuromonas | Desulfuromonas | Desulfuromusa | Dethiobacter | Dethiosulfatarculus |
| sp. TF | thiophila | kysingii | alkaliphilus | sandiegensis |
| Dethiosulfatibacter | Dethiosulfovibrio | Dethiosulfovibrio | Devosia | Devosia |
| aminovorans | peptidovorans | salsuginis | chinhatensis | crocina |
| Devosia | Devosia | Devosia | Devosia | Devosia |
| enhydra | epidermidihirudinis | geojensis | insulae | limi |
| Devosia | Devosia | Devosia | Devosia | Devosia |
| lucknowensis | psychrophila | riboflavina | soli | sp. 17-2-E-8 |
| Devosia | Devosia | Devosia | Devosia | Devosia |
| sp. A16 | sp. DBB001 | sp. H5989 | sp. LC5 | sp. Leaf420 |
| Devosia | Devosia | Devosia | Devosia | Devosia |
| sp. Leaf64 | sp. Root105 | sp. Root413D1 | sp. Root436 | sp. Root635 |
| Devosia | Devosia | Devosia | Devriesea | Dialister |
| sp. Root685 | sp. S37 | sp. YR412 | agamarum | invisus |
| Dialister | Dialister | Dialister | Diaphorobacter | Diaphorobacter |
| micraerophilus | pneumosintes | succinatiphilus | polyhydroxybutyrativoruns | sp. J5-51 |
| Dichelobacter | Dichomitus | Dickeya | Dickeya | Dickeya |
| nodosus | squalens | chrysanthemi | dadantii | dianthicola |
| Dickeya | Dickeya | Dickeya | Dickeya | Dickeya |
| paradisiaca | solani | sp. 2B12 | sp. NCPPB 3274 | sp. S1 |
| Dickeya | Dictyoglomus | Dictyoglomus | Dictyostelium | Dictyostelium |
| zeae | thermophilum | turgidum | discoideum | fasciculatum |
| Dictyostelium | Didymococcus | Dielma | Dietzia | Dietzia |
| purpureum | colitermitum | fastidiosa | alimentaria | cinnamea |
| Dietzia | Dietzia | Dietzia | Dietzia | Dinoroseobacter |
| sp. 111N12-1 | sp. HMSC21D01 | sp. UCD-THP | timorensis | shibae |
| Diplodia | Diplorickettsia | Dissulfuribacter | Dokdonella | Dokdonella |
| corticola | massiliensis | thermophilus | immobilis | koreensis |
| Dokdonia | Dokdonia | Dokdonia | Dokdonia | Dokdonia |
| donghaensis | pacifica | sp. 4H-3-7-5 | sp. MED134 | sp. PRO95 |
| Dolichospermum | Dolosicoccus | Dolesigranulum | Domibacillus | Domibacillus |
| circinale | paucivorans | pigrum | aminovorans | antri |
| Domibacillus | Domibacillus | Domibacillus | Domibacillus | Domibacillus |
| enclensis | epiphyticus | indicus | iocasae | mangrovi |
| Domibacillus | Domibacillus | Donghicola | Dongia | Dorea |
| robiginosus | tundrae | sp. KarMa | sp. URHE0060 | formicigenerans |
| Dorea | Dorea | Dorea | Dorea | Draconibacterium |
| longicatena | sp. 5-2 | sp. AGR2135 | sp. D27 | orientale |
| Draconibacterium | Drancourtella | Drancourtella | Drancourtella | Drancourtella |
| sediminis | massiliensis | sp. An12 | sp. An177 | sp. An210 |
| Drancourtella | Dubosiella | Duganella | Duganella | Duganella |
| sp. An57 | newyorkensis | phyllosphaerae | sacchari | sp. CF402 |
| Dunganella | Duganella | Duganella | Duganella | Duganella |
| sp. CF458 | sp. CF517 | sp. HH101 | sp. HH105 | sp. Leaf126 |
| Duganella | Duganella | Duganella | Duganella | Duganella |
| sp. Leaf61 | sp. Root1480D1 | sp. Root198D2 | sp. Root336D2 | zoogloeoides |
| Duodenibacillus | Dyadobacter | Dyadobacter | Dyadobacter | Dyadobacter |
| massiliensis | alkalitolerans | beijingensis | crusticola | fermentans |
| Dyadobacter | Dyadobacter | Dyadobacter | Dyadobacter | Dyadobacter |
| koreensis | psychrophilus | soli | sp. Leaf189 | sp. SG02 |
| Dyadobacter | Dyella | Dyella | Dyella | Dyella |
| tibetensis | ginsengisoli | japonica | jiangningensis | sp. OK004 |
| Dyella | Dyella-like sp. DHo | Dysgonomonas | Dysgonomonas | Dysgonomonas |
| thiooxydans | | capnocytophagoides | gadei | macrotermitis |
| Dysgonomonas | Dysgonomonas | Dysgonomonas | Echinicola | Echinicola |
| mossii | sp. BGC7 | sp. HGC4 | pacifica | vietnamensis |
| Ectothiorhodosinus | Ectothiorhodospira | Ectothiorhodospira | Ectothiorhodospira | Ectothiorhodospira |
| mongolicus | haloalkaliphila | magna | marina | mobilis |
| Ectothiorhodospira | Ectothiorhodospira | Edaphobacillus | Edaphobacter | Edwardsiella |
| sp. BSL-9 | sp. PHS-1 | lindanitolerans | aggregans | anguillarum |
| Edwardsiella | Edwardsiella | Edwardsiella | Edwardsiella | Edwardsiella |
| hoshinae | ictaluri | phage PEi20 | piscicida | sp. EA181011 |
| Edwardsiella | Effusibacillus | Eggerthella | Eggerthella | Eggerthella |
| tarda | pohliae | lenta | sp. 1_3_56FAA | sp. HGA1 |
| Eggerthella | Eggerthellaceae | Eggerthia | Ehrlichia | Ehrlichia |
| sp. YY7918 | bacterium AT8 | catenaformis | canis | chaffeensis |
| Ehrlichia | Ehrlichia | Ehrlichia | Eikenella | Eikenella |
| minasensis | ruminantium | sp. HF | corrodens | sp. NML02-A-017 |
| Eikenella | Eikenella | Eikenella | Eimeria | Eimeria |
| sp. NML03-A-027 | sp. NML120348 | sp. NML130454 | acervulina | maxima |
| Eimeria | Eimeria | Eimeria | Eisenbergiella | Eisenibacter |
| mitis | necatrix | tenella | tayi | elegans |
| Ekhidna | Elioraea | Elizabethkingia | Elizabethkingia | Elizabethkingia |
| lutea | tepidiphila | anophelis | endophytica | genomosp. 3 |
| Elizabethkingia | Elizabethkingia | Elizabethkingia | Elizabethkingia | Elizabethkingia |
| genomosp. 4 | meningoseptica | miricola | sp. C1558 | sp. F8124 |
| Elstera | Elstera | Elusimicrobium | Elusimicrobium | Emergencia |
| litoralis | sp. TH019 | minutum | sp. An273 | timonensis |
| Emiliania | Empedobacter | Empedobacter | Emticicia | Endocarpon |
| huxleyi | brevis | falsenii | sp. MM | pusilium |
| Endomicrobium | endosymbiont of | endosymbiont of | endosymbiont of | endosymbiont of |
| proavitum | Acanthamoeba | Bathymodiolus | Ridgeia piscesae | Tevnia jerichonana |
| | sp. UWC8 | sp. | | |
| endosymbiont of | endosymbiont ‘TC1’ of | Endozoicomonas | Endozoicomonas | Endozoicomonas |
| unidentified scaly snail | Trimyema compression | arenosclerae | ascidiicola | atrinae |
| isolate Monju | | | | |
| Endozoicomonas | Endozoicomonas | Endozoicomonas | Endozoicomonas | Enhydrobacter |
| elysicola | montiporae | numazuensis | sp. S-B4-1U | aerosaccus |
| Enhydrobacter | Enorma | Ensifer | Ensifer | Ensifer |
| sp. H5 | massiliensis | adhaerens | aridi | glycinis |
| Ensifer | Ensifer | Ensifer | Ensifer | Ensifer |
| shofinae | sojae | sp. 1H6 | sp. BR816 | sp. LC163 |
| Ensifer | Ensifer | Ensifer | Ensifer | Ensifer |
| sp. LCM 4579 | sp. Root 127 | sp. Root278 | sp. Root31 | sp. Root423 |
| Ensifer | Ensifer | Ensifer | Ensifer | Ensifer |
| sp. USDA 6670 | sp. WSM1721 | sp. Y1C4027 | sp. YR511 | sp. ZNC0028 |
| Entamoeba | Entamoeba | Entamoeba | Entamoeba | Enteractinococcus |
| dispar | histolytica | invadens | nuttalli | helveticum |
| Enterobacter | Enterobacter | Enterobacter | Enterobacter | Enterobacter |
| asburiae | bugandensis | cancerogenus | cloacae | cloacae |
| | | | | complex ‘Hoffmann |
| | | | | cluster III’; |
| Enterobacter | Enterobacter | Enterobacter | Enterobacter | Enterobacter |
| cloacae | cloacae | cloacae | cloacae | cloacae |
| complex ‘Hoffmann | complex sp. 35009 | complex sp. 35669 | complex sp. 35699 | complex sp. GN02208 |
| cluster IV’ | | | | |
| Enterobacter | Enterobacter | Enterobacter | Enterobacter | Enterobacter |
| cloacae | cloacae | cloacae | cloacae | cloacae |
| complex sp. GN02283 | complex sp. GN02548 | complex sp. GN02693 | complex sp. GN03164 | complex sp. GN03842 |
| Enterobacter | Enterobacter | Enterobacter | Enterobacter | Enterobacter |
| cloacae | cloacae | cloacae | cloacae | cloacae |
| complex sp. GN04363 | complex sp. GN04787 | complex sp. GN05526 | complex sp. GN05729 | complex sp. GN05753 |
| Enterobacter | Enterobacter | Enterobacter | Enterobacter | Enterobacter |
| cloacae | cloacae | cloacae | cloacae | hormaechei |
| complex sp. GN06232 | complex sp. SMART_454 | complex sp. SMART_763 | complex sp. SMART_930 | |
| Enterobacter kobei | Enterobacter | Enterobacter ludwigii | Enterobacter | Enterobacter mori |
| | lignolyticus | | massiliensis | |
| Enterobacter phage | Enterobacter soli | Enterobacter | Enterobacter | Enterobacter |
| Enc34 | | sp. 10-1 | sp. 50588862 | sp. 50858885 |
| Enterobacter | Enterobacter | Enterobacter | Enterobacter | Enterobacter |
| sp. 638 | sp. Ag1 | sp. BIDMC 26 | sp. BIDMC 29 | sp. BIDMC 30 |
| Enterobacter | Enterobacter | Enterobacter | Enterobacter | Enterobacter |
| sp. BIDMC87 | sp. BIDMC92 | sp. B1DMC99 | sp. Bisph1 | sp. Bisph2 |
| Enterobacter | Enterobacter | Enterobacter | Enterobacter | Enterobacter |
| sp. BWH64 | sp. DC1 | sp. EGD-HP1 | sp. FY-07 | sp. GN02600 |
| Enterobacter | Enterobacter | Enterobacter | Enterobacter | Enterobacter |
| sp. HMSC055A11 | sp. HMSC16D10 | sp. 1F2SW-B1 | sp. 1F2SW-P2 | sp. J49 |
| Enterobacter | Enterobacter | Enterobacter | Enterobacter | Enterobacter |
| sp. kpr-6 | sp. ku-bf2 | sp. MGH 24 | sp. MGH 38 | sp. MGH120 |
| Enterobacter | Enterobacter | Enterobacter | Enterobacter | Enterobacter |
| sp. NFIX45 | sp. NFIX58 | sp. NFIX59 | sp. NFR05 | sp. PDC34 |
| Enterobacter | Enterobacter | Enterobacter | Enterobacter | Enterobacter |
| sp. R4-368 | sp. Sa187 | sp. SENG-6 | sp. T1-1 | sp. TUM13743 |
| Enterobacter | Enterobacter | Enterobacter | Enterobacter virus | Enterobacter |
| sp. UCD-UG_FMILLET | sp. ZOR0014 | timonensis | PG7 | xiangfangensis |
| Enterobacteria phage | Enterobacteriaceae | Enterobacteriaceae | Enterobacteriaceae | Enterobacteriaceae |
| SfV | bacterium 9_2_54FAA | bacterium ATCC 29904 | bacterium B14 | bacterium LSJC7 |
| Enterobacteriaceae | Enterococcus | Enterococcus asini | Enterococcus avium | Enterococcus caccae |
| bacterium strain FGI 57 | aquimarinus | | | |
| Enterococcus | Enterococcus canis | Enterococcus | Enterococcus cecorum | Enterococcus |
| canintestini | | casseliflavus | | columbae |
| Enterococcus devriesei | Enterococcus dispar | Enterococcus durans | Enterococcus faecalis | Enterococcus faecium |
| Enterococcus | Enterococcus gilvus | Enterococcus | Enterococcus | Enterococcus hirae |
| gallinarum | | haemoperoxidus | hermanniensis | |
| Enterococcus italicus | Enterococcus | Enterococcus | Enterococcus | Enterococcus mundtii |
| | malodoratus | massiliensis | moraviensis | |
| Enterococcus pallens | Enterococcus pernyi | Enterococcus phage | Enterococcus phage | Enterococcus |
| | | IME-EFm1 | IME-EFm5 | phoeniculicola |
| Enterococcus | Enterococcus | Enterococcus | Enterococcus | Enterococcus ratti |
| plantarum | pseudoavium | quebecensis | raffinosus | |
| Enterococcus rivorum | Enterococcus | Enterococcus | Enterococcus | Enterococcus |
| | saccharolyticus | silesiacus | sp. 10A9_DIV0425 | sp. 12C11_DIV0727 |
| Enterococcus | Enterococcus | Enterococcus | Enterococcus | Enterococcus |
| sp. 3C7_DIV0644 | sp. 3G1_DIV0629 | sp. 3G6_DIV0642 | sp. 3H8_DIV0648 | sp. 4E1_DIV0656 |
| Enterococcus | Enterococcus | Enterococcus | Enterococcus | Enterococcus |
| sp. 4G2_DIV0659 | sp. 5B3_DIV0040 | sp. 6C8_DIV0013 | sp. 6D12_DIV0197 | sp. 7F3_DIV0205 |
| Enterococcus | Enterococcus | Enterococcus | Enterococcus | Enterococcus |
| sp. 8G7_MSG3316 | sp. 9D6_DIV0238 | sp. 9E7_D1V0242 | sp. C1 | sp. HMSC05C03 |
| Enterococcus | Enterococcus | Enterococcus | Enterococcus | Enterococcus |
| sp. HMSC063D12 | sp. HMSC063H10 | sp. HMSC069A01 | sp. HMSC070F12 | sp. HMSC072F07 |
| Enterococcus | Enterococcus | Enterococcus | Enterococcus | Enterococcus |
| sp. HMSC072H05 | sp. HMSC29A04 | sp. kppr-6 | sp. Marseille-P2817 | sp. MN05 |
| Enterococcus | Enterococcus | Enterococcus sulfureus | Enterococcus termitis | Enterococcus |
| sp. RIT-PI-f | sp. TR | | | thailandicus |
| Enterococcus | Enterococcus | Enterococcus villorum | Enterocytozoon | Enterorhabdus |
| ureasiticus | ureilyticus | | bieneusi | caecimuris |
| Enterorhabdus | Enterovibrio calviensis | Enterovibrio coralii | Enterovibrio nigricans | Enterovibrio |
| mucosicola | | | | norvegicus |
| Enterovibrio paciflcus | Entomoplasma | Entomoplasma | Entomoplasma | Entomoplasma |
| | lucivorax | luminosum | melaleucae | somnilux |
| Epibacterium ulvae | Epulopiscium | Eremococcus | Eremothecium | Eremothecium gossypii |
| | sp. ‘N.t. morphotype B’ | coleocola | cymbalariae | |
| Eremothecium | Erwinia amylovora | Erwinia billingiae | Erwinia gerundensis | Erwinia iniecta |
| sinecaiidum | | | | |
| Erwinia mallotivora | Erwinia oleae | Erwinia persicina | Erwinia phage PEp14 | Erwinia phage |
| | | | | PhiEaH1 |
| Erwinia phage | Erwinia | Erwinia pyrifoliae | Erwinia | Erwinia |
| F phiEaH2 | piriflorinigrans | | sp. 9145 | sp. Ejp617 |
| Erwinia | Erwinia | Erwinia tasmaniensis | Erwinia teleogrylli | Erwinia toletana |
| sp. ErVv1 | sp. Leaf53 | | | |
| Erwinia tracheiphila | Erwinia typographi | Erwinia virus Ea35-70 | Erysipelatoclostridium | Erysipelatoclostridium |
| | | | ramosum | sp. An15 |
| Erysipelatoclostridium | Erysipelothrix larvae | Erysipelothrix | Erysipelothrix | Erysipelotrichaceae |
| sp. An173 | | rhusiopathiae | tonsillarum | bacterium 2_2_44A |
| Erysipelotrichaceae | Erysipelotrichaceae | Erysipelotrichaceae | Erysipelotrichaceae | Erysipelotrichaceae |
| bacterium 21_3 | bacterium 5_2_54FAA | bacterium 6_1_45 | bacterium 146 | bacterium MTC7 |
| Erysipelotrichaceae | Erythrobacter | Erythrobacter citreus | Erythrobacter flavus | Erythrobacter |
| bacterium NK3D112 | atlanticus | | | gangjinensis |
| Erythrobacter litoralis | Erythrobacter longus | Erythrobacter luteus | Erythrobacter marinus | Erythrobacter |
| | | | | nanhaisediminis |
| Erythrobacter | Erythrobacter | Erythrobacter | Erythrobacter | Erythrobacter |
| sp. AP23 | sp. CCH5-A1 | sp. EhN03 | sp. HI0063 | sp. HI00D59 |
| Erythrobacter | Erythrobacter | Erythrobacter | Erythrobacter | Erythrobacter |
| sp. HL-111 | sp. JL475 | sp. NAP1 | sp. QSSC1-22B | sp. SD-21 |
| Erythrobacter | Erythrobacter | Erythrobacte vulgarisr | Erythrobacteraceae | Escherichia albertii |
| sp. SG61-1L | sp. YT30 | | bacterium CCH12-C2 | |
| Escherichia coli | Escherichia fergusonii | Escherichia marmotae | Escherichia | Escherichia |
| | | | sp. 1_1_43 | sp. 3_2_53FAA |
| Escherichia | Escherichia | Escherichia | Escherichia | Escherichia |
| sp. 4_1_40B | sp. B1 147 | sp. KTE11 | sp. KTE114 | sp. KTE159 |
| Escherichia | Escherichia | Escherichia | Escherichia | Escherichia |
| sp. KTE31 | sp. KTE52 | sp. KTE96 | sp. TW09231 | sp. TW09276 |
| Escherichia | Escherichia | Escherichia | Escherichia | Escherichia |
| sp. TW09308 | sp. TW10509 | sp. TW11588 | sp. TW14182 | sp. TW15838 |
| Escherichia virus 186 | Escherichia virus | Escherichia virus | Escherichia virus T5 | Escherichia vulneris |
| | Cajan | FFH2 | | |
| Ethanoligenens | Eubacteriaceae | Eubacteriaceae | Eubacterium | Eubacterium barkeri |
| harbinense | bacterium CHKC1004 | bacterium CHKC1005 | aggregans | |
| Eubacterium | Eubacterium | Eubacterium limosum | Eubacterium | Eubacterium |
| callanderi | coprostanoligenes | | oxidoreducens | plexicaudatum |
| Eubacterium | Eubacterium ramulus | Eubacterium | Eubacterium | Eubacterium |
| pyruvativorans | | ruminantium | sp. 14-2 | sp. 3_1_31 |
| Eubacterium | Eubacterium | Eubacterium | Eubacterium | Eubacterium |
| sp. 68-3-10 | sp. AB3007 | sp. An11 | sp. An3 | sp. ER2 |
| Eubacterium | Eubacterium uniforme | Eubacterium | Eubacterium | Eudoraea adriatica |
| sp. SB2 | | ventriosum | xylanophilum | |
| Euryhalocaulis | Eutypa lata | Ewingella americana | Exiguobacterium | Exiguobacterium |
| caribicus | | | acetylicum | alkaliphilum |
| Exiguobacterium | Exiguobacterium | Exiguobacterium | Exiguobacterium | Exiguobacterium |
| antarcticum | aurantiacum | chiriqhucha | indicum | marinum |
| Exiguobacterium | Exiguobacterium | Exiguobacterium | Exiguobacterium | Exiguobacterium |
| mexicanum | oxidotolerans | profundum | sibiricum | sp. AB2 |
| Exiguobacterium | Exiguobacterium | Exiguobacterium | Exiguobacterium | Exiguobacterium |
| sp. AT1b | sp. BMC-KP | sp. HVEsp1 | sp. JLM-2 | sp. KKBO11 |
| Exiguobacterium | Exiguobacterium | Exiguobacterium | Exiguobacterium | Exiguobacterium |
| sp. KRL4 | sp. Leaf187 | sp. NG55 | sp. RIT341 | sp. S17 |
| Exiguobacterium | Exiguobacterium | Exiguobacterium | Exiguobacterium | Exiguobacterium |
| sp. SH31 | sp. U13-1 | sp. ZOR0005 | sp. ZWU0009 | undae |
| Exophiala atiuamarina | Exophiala dermatitidis | Exophiala mesophila | Exophiala oligosperma | Exophiala spinifera |
| Exophiala xenobiotica | Ezakiella massiliensis | Fabibacter misakiensis | Fabibacter pacificus | Fabibacter |
| | | | | sp. 4D4 |
| Facklamia hominis | Facklamia ignava | Facklamia languida | Facklamia miroungae | Facklamia sourekii |
| Facklamia | Faecalibacterium | Faecalibacterium | Faecalibacterium | Faecalibacterium |
| sp. HMSC062C11 | prausnitzii | sp. An121 | sp. An122 | sp. An192 |
| Faecalibacterium | Faecalibacterium | Faecalibaciilum | Faecalicatena contorta | Faecalicatena |
| sp. An58 | sp. An77 | rodentium | | fissicatena |
| Faecalicoccus | Faecalitalea | Falsirhodobacter | Fangia hongkongensis | Fenollaria massiliensis |
| pleomorphus | cylindroides | sp. alg1 | | |
| Fenollaria timonensis | Fermentimonas | Ferrimicrobium | Ferrimonas balearica | Ferrimonas fiittsuensis |
| | caenicola | acidiphilum | | |
| Ferrimonas | Ferrimonas marina | Ferrimonas sediminum | Ferrimonas senticii | Ferriphaselus |
| kyonanensis | | | | amnicola |
| Ferriphaselus | Ferrithrix | Ferrovum myxofaciens | Ferrovum | Ferrovum |
| sp. R-1 | thermotolerans | | sp. JA12 | sp. PN-J185 |
| Fervidicella | Fervidicola | Fervidobacterium | Fervidobacterium | Fervidobacterium |
| metallireducens | ferrireducens | gondwanense | islandicum | nodosum |
| Fervidobacterium | Fervidobacterium | Fibrella aestuarina | Fibrella | Fibrisoma limi |
| pennivorans | thailandensis | | sp. ES10-3-2-2 | |
| Fibrobacter | Fibrobacter | Fibrobacter | Fibrobacter | Fibrobacter |
| intestinalis | sp. UWB1 | sp. UWB11 | sp. UWB12 | sp. UWB13 |
| Fibrobacter | Fibrobacter | Fibrobacter | Fibrobacter | Fibrobacter |
| sp. UWB15 | sp. UWB2 | sp. UWB3 | sp. UWB4 | sp. UWB5 |
| Fibrobacter | Fibrobacter | Fibrobacter | Fibrobacter | Fibrobacter |
| sp. UWB17 | sp. UWB8 | sp. UWCM | sp. UWEL | sp. UWH1 |
| Fibrobacter | Fibrobacter | Fibrobacter | Fibrobacter | Fibrobacter |
| sp. UWH3 | sp. UWH4 | sp. UWH5 | sp. UWH6 | sp. UWH9 |
| Fibrobacter | Fibrobacter | Fibrobacter | Fibrobacter | Fibrobacter |
| sp. UWOS | sp. UWOV1 | sp. UWP2 | sp. UWR2 | sp. UWR3 |
| Fibrobacter | Fibrobacter | Fibroporia radiculosa | Fictibacillus arsenicus | Fictibacillus enclensis |
| sp. UWT2 | succinogenes | | | |
| Fictibacillus gelatini | Fictibacillus | Fictibacillus | Fictibacillus solisalsi | Fictibacillus |
| | macauensis | phosphorivorans | | sp. FJAT-27399 |
| Fictibacillus | filamentous | Filifactor alocis | Filimonas lacunae | Fimbriiglobus ruber |
| sp. GDSW-R2A3 | cyanobacterium ESFC-1 | | | |
| Fimbriimonas | Finegoldia magna | Firmicutes bacterium | Firmicutes bacterium | Firmicutes bacterium |
| ginsengisoli | | ASF500 | M10-2 | ZOR0006 |
| Fischerella muscicola | Fischerella | Fischerella | Fischerella | Flagellimonas |
| | sp. PCC 9339 | sp. PCC 9431 | sp. PCC 9605 | eckloniae |
| Flammeovirga paciflca | Flammeovirga | Flammeovirga | Flammeovirga | Flammeovirgaceae |
| | sp. MY04 | sp. OC4 | sp. SJP92 | bacterium 311 |
| Flaviflexus | Flavihumibacter | Flavihumibacter | Flavihumibacter | Flaviramulus basaltis |
| massiliensis | petaseus | solisilvae | sp. ZG627 | |
| Flaviramulus | Flavisolibacter | Flavisolibacter | Flavobacteria | Flavobacteria |
| ichthyoenteri | ginsengisoli | sp. LCS9 | bacterium BAL38 | bacterium BBFL7 |
| Flavobacteria | Flavobacteria | Flavobacteriaceae | Flavobacteriaceae | Flavobacteriaceae |
| bacterium MS024-2A | bacterium MS024-3C | bacterium 3519-10 | bacterium A100 | bacterium CP2B |
| Flavobacteriaceae | Flavobacteriaceae | Flavobacteriaceae | Flavobacteriaceae | Flavobacteriaceae |
| bacterium CRH | bacterium HSG9 | bacterium JJC | bacterium | bacterium UJ101 |
| | | | MAR_2010_188 | |
| Flavobacteriales | Flavobacterium | Flavobacterium | Flavobacterium | Flavobacterium |
| bacterium ALC-1 | akiainvivens | anhuiense | antarcticum | aituatile |
| Flavobacterium | Flavobacterium | Flavobacterium | Flavobacterium | Flavobacterium caeni |
| aquidurense | araucananum | beibuense | branchiophilum | |
| Flavobacterium | Flavobacterium | Flavobacterium | Flavobacterium | Flavobacterium |
| cauense | chilense | chungangense | columnare | communis |
| Flavobacterium | Flavobacterium | Flavobacterium | Flavobacterium | Flavobacterium |
| crassostreae | cucumis | daejeonense | defluvii | degerlachei |
| Flavobacterium | Flavobacterium | Flavobacterium | Flavobacterium | Flavobacterium |
| denitrificans | enshiense | filum | flevense | fluvii |
| Flavobacterium | Flavobacterium | Flavobacterium | Flavobacterium | Flavobacterium |
| fontis | frigidarium | frigoris | gelidilacus | gillisiae |
| Flavobacterium | Flavobacterium | Flavobacterium | Flavobacterium | Flavobacterium |
| gilvum | glycines | granuli | haoranii | hercynium |
| Flavobacterium | Flavobacterium | Flavobacterium | Flavobacterium | Flavobacterium |
| hibernum | hydatis | indicium | johnsoniae | limnosediminis |
| Flavobacterium | Flavobacterium | Flavobacterium | Flavobacterium | Flavobacterium |
| marinum | micromati | noncentrifugens | omnivorum | oncorhynchi |
| Flavobacterium | Flavobacterium | Flavobacterium | Flavobacterium | Flavobacterium |
| pectinovorum | phage 11b | phragmitis | piscis | plurextorum |
| Flavobacterium | Flavobacterium | Flavobacterium | Flavobacterium | Flavobacterium |
| psychrophilum | reichenbachii | rivuli | saccharophilum | saliperosum |
| Flavobacterium | Flavobacterium | Flavobacterium | Flavobacterium | Flavobacterium |
| sasangense | segetis | seoulense | sinopsychrotolerans | soli |
| Flavobacterium | Flavobacterium | Flavobacterium | Flavobacterium | Flavobacterium |
| sp. 316 | sp. 83 | sp. A45 | sp. ABG | sp. ACAM 123 |
| Flavobacterium | Flavobacterium | Flavobacterium | Flavobacterium | Flavobacterium |
| sp. AED | sp. AJR | sp. B17 | sp. CF136 | sp. F52 |
| Flavobacterium | Flavobacterium | Flavobacterium | Flavobacterium | Flavobacterium |
| sp. Fl | sp. FPG59 | sp. IR1 | sp. JRM | sp. KBS0721 |
| Flavobacterium | Flavobacterium | Flavobacterium | Flavobacterium | Flavobacterium |
| sp. KJJ | sp. KMS | sp. Leaf359 | sp. Leaf82 | sp. LM4 |
| Flavobacterium | Flavobacterium | Flavobacterium | Flavobacterium | Flavobacterium |
| sp. LM5 | sp. MEB061 | sp. ov086 | sp. Root186 | sp. Root420 |
| Flavobacterium | Flavobacterium | Flavobacterium | Flavobacterium | Flavobacterium |
| sp. Root901 | sp. Root935 | sp. TAB 87 | sp. TH021 | sp. TH167 |
| Flavobacterium | Flavobacterium | Flavobacterium | Flavobacterium | Flavobacterium |
| sp. URHB0058 | sp. WG21 | spartansii | subsaxonicum | succinicans |
| Flavobacterium | Flavobacterium | Flavobacterium | Flavobacterium | Flavobacterium |
| suncheonense | swingsii | tegetincola | terrae | terrigena |
| Flavobacterium | Flavobacterium | Flavobacterium | Flavobacterium | Flavobacterium |
| ummariense | urocaniciphilum | xanthum | xinjiangense | xueshanense |
| Flavonifractor | Flavonifractor | Flavonifractor | Flavonifractor | Flavonifractor |
| plautii | sp. An10 | sp. An100 | sp. An112 | sp. An135 |
| Flavonifractor | Flavonifractor | Flavonifractor | Flavonifractor | Flavonifractor |
| sp. An306 | sp. An4 | sp. An52 | sp. An82 | sp. An9 |
| Flavonifractor | Flavonifractor | Flectobacillus | Flectobacillus | Flexibacter |
| sp. An91 | sp. An92 | major | sp. BAB-3569 | flexilis |
| Flexilinea | Flexistipes | Flexithrix | Fluoribacter | Fluoribacter |
| flocculi | sinusarabici | dorotheae | bozemanae | dumoffii |
| Fluoribacter | Fluviicola | Fodinicurvata | Fodinicurvata | Fomitiporia |
| gormanii | taffensis | fenggangensis | sediminis | mediterranea |
| Fonsecaea | Fonsecaea | Fonsecaea | Fontibacillus | Fontimonas |
| erecta | multimorphosa | pedrosoi | panacisegetis | thermophila |
| Formivibrio | Formosa | Formosa | Formosa | Formosa |
| citricus | agariphila | algae | haliotis | sp. Hel1_31_208 |
| Formosa | Fortiea | Fournierella | Francisella | Francisella |
| sp. Hel1_33_131 | contorta | massiliensis | halioticida | noatunensis |
| Francisella | Francisella | Francisella | Francisella | Francisella |
| persica | philomiragia | sp. CA97-1460 | sp. FSC1006 | sp. MA067296 |
| Francisella | Francisella | Francisella | Francisella | Francisella |
| sp. TX076608 | sp. TX077308 | sp. TX077310 | sp. W12- | tularensis |
| | | | 1067 | |
| Franconibacter | Franconibacter | Frankia | Frankia | Frankia |
| helveticus | pulveris | alni | discariae | elaeagni |
| Frankia | Frankia | Frankia | Frankia | Frankia |
| inefficax | sp. ACN1ag | sp. BMG5.36 | sp. Cc117 | sp. Cc149 |
| Frankia | Frankia | Frankia | Frankia | Frankia |
| sp. DC12 | sp. EAN1pec | sp. EI5c | sp. EUN1f | sp. G2 |
| Frankia | Frankia | Frankia | Frankia | Frankia |
| sp. Iso899 | sp. KB5 | sp. M16386 | sp. NRRL B- | sp. QA3 |
| | | | 16219 | |
| Frankia | Frankia symbiont of | Frankineae bacterium | Frateuria | Frateuria |
| sp. R43 | Datisca glomerata | MT45 | aurantia | sp. Soil773 |
| Frateuria | Friedmanniella | Friedmanniella | Friedmanniella | Frigoribacterium |
| terrea | flava | luteola | sagamiharensis | sp. JB110 |
| Frigoribacterium | Frigoribacterium | Frigoribacterium | Frigoribacterium | Frigoribacterium |
| sp. Leaf164 | sp. Leaf172 | sp. Leaf186 | sp. Leaf263 | sp. Leaf44 |
| Frigoribacterium | Frigoribacterium | Frigoribacterium | Frigoribacterium | Frischella |
| sp. Leaf8 | sp. MCBA15 019 | sp. MEB024 | sp. RIT-PI-h | perrara |
| Frondihabitans | Frondihabitans | Fructobacillus | Fructobacillus | Fructobacillus |
| sp. Leaf304 | sp. PAMC 28766 | durionis | ficulneus | fructosus |
| Fructobacillus | Fructobacillus | Fructobacillus | Fuerstia | Fulvimarina |
| pseudoficulneus | sp. EFB-N1 | tropaeoli | marisgermanicae | manganoxydans |
| Fulvimarina | Fulvivirga | Fusarium | Fusarium | Fusarium |
| pelagi | imtechensis | graminearum | oxysporum | pseudograminearum |
| Fusarium | Fusibacter | Fusicatenibacter | Fusicatenibacter | Fusobacterium |
| verticillioides | sp. 3D3 | saccharivorans | sp. 2789STDY5834925 | equinum |
| Fusobacterium | Fusobacterium | Fusobacterium | Fusobacterium | Fusobacterium |
| gonidiaformans | hwasookii | massiliense | mortiferum | necrophorum |
| Fusobacterium | Fusobacterium | Fusobacterium | Fusobacterium | Fusobacterium |
| nucleatum | perfoetens | periodonticum | russii | sp. CM21 |
| Fusobacterium | Fusobacterium | Fusobacterium | Fusobacterium | Fusobacterium |
| sp. CM22 | sp. HMSC064B11 | sp. HMSC064B12 | sp. HMSC065F01 | sp. HMSC073F01 |
| Fusobacterium | Fusobacterium | Fusobacterium | Fusobacterium | Fusobacterium |
| sp. OBRC1 | sp. oral | sp. oral | ulcerans | varium |
| | taxon 203 | taxon 370 | | |
| Gabonia | Gabonibacter | Gaetbulibacter | Gaeumannomyces | Galbibacter |
| massiliensis | massiliensis | saemankumensis | tritici | marinus |
| Gallaecimonas | Gallaecimonas | Gallibacterium | Gallibacterium | Gallibacterium |
| pentaromativorans | xiamenensis | anatis | genomosp. 1 | genomosp. 2 |
| Gallibacterium | Gallibacterium | Gallionella | gamma | gamma |
| genomosp. 3 | salpingitidis | capsiferriformans | proteobacterium | proteobacterium HdN1 |
| | | | BDW918 | |
| gamma | gamma | gamma | gamma | gamma |
| proteobacterium | proteobacterium | proteobacterium | proteobacterium | proteobacterium |
| HIMB30 | HIMB55 | HTCC2207 | HTCC5015 | IMCC1989 |
| gamma | gamma | gamma | gamma | Gammaproteobacteria |
| proteobacterium | proteobacterium L18 | proteobacterium | proteobacterium | bacterium 2W06 |
| IMCC3088 | | NOR5-3 | WG36 | |
| Gammaproteobacteria | Gammaproteobacteria | Garciella | Gardnerella | Gardnerella |
| bacterium MFB021 | bacterium MOLA455 | nitratireducens | sp. 30-4 | vaginalis |
| Gayadomonas | Geitlerinema | Geitlerinema | Geitlerinema | Gelidibacter |
| joobiniege | sp. PCC | sp. PCC | sp. PCC | algens |
| | 7105 | 7407 | 9228 | |
| Gelidibacter | Gemella | Gemella | Gemella | Gemella |
| mesophilus | asaccharolytica | bergeri | cuniculi | haemolysans |
| Gemella | Gemella | Gemella | Geminicoccus | Geminocystis |
| morbillorum | sanguinis | sp. Marseille- | roseus | herdmanii |
| | | P3249 | | |
| Geminocystis | Geminocystis | Gemmata | Gemmata | Gemmatimonas |
| sp. NIES-3708 | sp. NIES-3709 | obscuriglobus | sp. SH-PL17 | aurantiaca |
| Gemmatimonas | Gemmiger | Gemmiger | Gemmiger | Gemmiger |
| phototrophica | formicilis | sp. An120 | sp. An194 | sp. An50 |
| Gemmiger | Gemmobacter | Gemmobacter | Gemmobacter | Gemmobacter |
| sp. An87 | aquatilis | megaterium | nectariphilus | sp. LW-1 |
| Geoalkalibacter | Geoalkalibacter | Geobacillus | Geobacillus | Geobacillus |
| ferrihydriticus | subterraneus | galactosidasius | genomosp. 3 | icigianus |
| Geobacillus | Geobacillus | Geobacillus | Geobacillus | Geobacillus |
| jurassicus | kaustophilus | lituanicus | phage GBK2 | sp. 44C |
| Geobacillus | Geobacillus | Geobacillus | Geobacillus | Geobacillus |
| sp. 46C-IIa | sp. 8 | sp. B4113 201601 | sp. C56-T3 | sp. G11MC16 |
| Geobacillus | Geobacillus | Geobacillus | Geobacillus | Geobacillus |
| sp. GHH01 | sp. LEMMY01 | sp. MAS1 | sp. WCH70 | sp. Y4.1MC1 |
| Geobacillus | Geobacillus | Geobacillus | Geobacillus | Geobacillus |
| stearothermophilus | subterraneus | thermocatenulatus | thermodenitrificans | thermoleovorans |
| Geobacillus virus E2 | Geobacillus virus E3 | Geobacillus | Geobacillus | Geobacter |
| | | vulcani | zalihae | anodireducens |
| Geobacter | Geobacter | Geobacter | Geobacter | Geobacter |
| bemidjiensis | bremensis | daltonii | lovleyi | metallireducens |
| Geobacter | Geobacter | Geobacter | Geobacter | Geobacter |
| pelophilus | pickeringii | soli | sp. DSM | sp. M18 |
| | | | 9736 | |
| Geobacter | Geobacter | Geobacter | Geobacter | Geobacter |
| sp. M21 | sp. OR-1 | sulfurreducens | thiogenes | uraniireducens |
| Geodermatophilaceae | Geodermatophilaceae | Geodermatophilaceae | Geodermatophilus | Geodermatophilus |
| bacterium URHA0031 | bacterium URHB0048 | bacterium URHB0062 | africanus | amargosae |
| Geodermatophilus | Geodermatophilus | Geodermatophilus | Geodermatophilus | Geodermatophilus |
| brasiliensis | nigrescens | obscurus | poikilotrophus | pulveris |
| Geodermatophilus | Geodermatophilus | Geodermatophilus | Geodermatophilus | Geodermatophilus |
| ruber | saharensis | siccatus | soli | sp. DSM 44208 |
| Geodermatophilus | Geodermatophilus | Geodermatophilus | Geodermatophilus | Geofilum |
| sp. DSM 45219 | sp. Leaf369 | taihuensis | telluris | rubicundum |
| Geofilum | Geoglobus | Geoglobus | Geomicrobium | Geomicrobium |
| sp. HF401 | acetivorans | ahangari | sp. JCM 19037 | sp. JCM 19038 |
| Geomicrobium | Geomicrobium | Geopsychrobacter | Geosporobacter | Geothermobacter |
| sp. JCM 19039 | sp. JCM 19055 | electrodiphilus | ferrireducens | sp. EPR-M |
| Geothrix | Geotoga | Geovibrio | Giesbergeria | Gilliamella |
| fermentans | petraea | sp. L21-Ace- | anulus | apicola |
| | | BES | | |
| Gilliamella | Gilliamella | Gilliamella | Gilliamella | Gilliamella |
| bombi | bombicola | intestini | mensalis | sp. A-4-12 |
| Gilliamella | Gilliamella | Gillisia | Gillisia | Gillisia |
| sp. A7 | sp. AM1 | limnaea | marina | sp. CAL575 |
| Gillisia | Gilvibacter | Gilvimarinus | Gilvimarinus | Gilvimarinus |
| sp. JM1 | sp. SZ-19 | agarilyticus | chinensis | polysaccharolyticus |
| Gimesia maris | Glaciecola | Glaciecola | Glaciecola | Glaciecola |
| | nitratireducens | pallidula | punicea | sp. HTCC2999 |
| Glaciibacter | Glarea | Globicatella | Globicatella | Globicatella |
| superstes | lozoyensis | sanguinis | sp. HMSC072A10 | sulfidifaciens |
| Gloeobacter | Gloeobacter | Gloeocapsa | Gloeocapsa | Gloeomargarita |
| kilaueensis | violaceus | sp. PCC 73106 | sp. PCC 7428 | lithophora |
| Gloeophyllum | Glomeribacter | Gluconacetobacter | Gluconacetobacter | Gluconobacter |
| trabeum | sp. 1016415 | diazotrophicus | sp. SXCC-1 | albidus |
| Gluconobacter | Gluconobacter | Gluconobacter | Gluconobacter | Gluconobacter |
| cerinus | frateurii | japonicus | morbifer | oxydans |
| Gluconobacter | Gluconobacter | Gluconobacter | Glutamicibacter | Glutamicibacter |
| roseus | sp. DsW_056 | sp. DsW_058 | arilaitensis | mysorens |
| Glutamicibacter | Glycomyces | Glycomyces | Glycomyces | Glycomyces |
| sp. 0426 | arizonensis | harbinensis | sambucus | sp. NRRL |
| | | | | B-16210 |
| Glycomyces | Glypta | Gordonia | Gordonia | Gordonia |
| tenuis | fumiferanae | aichiensis | alkanivorans | amarae |
| | ichnovirus | | | |
| Gordonia | Gordonia | Gordonia | Gordonia | Gordonia |
| amicalis | araii | bronchialis | desulfuricans | effusa |
| Gordonia | Gordonia | Gordonia | Gordonia | Gordonia |
| hirsuta | hydrophobica | jacobaea | kroppenstedtii | lacunae |
| Gordonia | Gordonia | Gordonia | Gordonia | Gordonia |
| malaquae | namibiensis | neofelifaecis | otitidis | paraffinivorans |
| Gordonia | Gordonia | Gordonia | Gordonia | Gordonia |
| polyisoprenivorans | rhizosphera | rubripertincta | shandongensis | sihwensis |
| Gordonia | Gordonia | Gordonia | Gordonia | Gordonia |
| soli | sp. 852002- | sp. 852002- | sp. 852002- | sp. 852002- |
| | 10350 SCH5691597 | 50395 SCH5434458 | 50816 SCH5313054-c | 51296 SCH5728562-b |
| Gordonia | Gordonia | Gordonia | Gordonia | Gordonia |
| sp. HS-NH1 | sp. i37 | sp. IITR100 | sp. LAM0048 | sp. NB4-1Y |
| Gordonia | Gordonia | Gordonia | Gordonia | Gordonia |
| sp. QH-11 | sp. QH-12 | sp. UCD- | terrae | virus Woes |
| | | TK1 | | |
| Gordonia | Gordonibacter | Gordonibacter | Gordonibacter | Gordonibacter |
| westfalica | massiliensis | pamelaeae | sp. An230 | sp. An232A |
| Gordonibacter | Gorillibacterium | Gorillibacterium | Gottschalkia | Gracilibacillus |
| urolithinfaciens | massiliense | sp. SN4 | acidurici | boraciitolerans |
| Gracilibacillus | Gracilibacillus | Gracilibacillus | Gracilibacillus | Gracilibacillus |
| halophilus | kekensis | lacisalsi | massiliensis | orientalis |
| Gracilibacillus | Gracilibacillus | Gracilimonas | Gramella | Gramella |
| timonensis | ureilyticus | tropica | echinicola | flava |
| Gramella | Gramella | Gramella | Gramella | Gramella |
| forsetii | portivictoriae | sp. LPB0144 | sp. MAR_2010_102 | sp. MAR_2010_147 |
| Granulibacter | Granulicatella | Granulicatella | Granulicatella | Granulicatella |
| bethesdensis | adiacens | balaenopterae | elegans | sp. HMSC30F09 |
| Granulicatella | Granulicella | Granulicella | Granulicella | Granulicella |
| sp. HMSC31F03 | mallensis | pectinivorans | rosea | tundricola |
| Granulicoccus | Granulosicoccus | Gregarina | Grimontia | Grimontia |
| phenolivorans | antarcticus | niphandrodes | celer | hollisae |
| Grimontia | Grimontia | Grimontia | Grosmannia | Gryllotalpicola |
| indica | marina | sp. AD028 | clavigera | ginsengisoli |
| Guillardia | Gulbenkiania | Gulbenkiania | Gulosibacter | Gulosibacter |
| theta | indica | mobilis | molinativorax | sp. 10 |
| Gynuella | Haematobacter | Haematobacter | Haematobacter | Haematomicrobium |
| sunshinyii | genomosp. 1 | massiliensis | missouriensis | sanguinis |
| Haematospirillum | Haemophilus | Haemophilus | Haemophilus | Haemophilus |
| jordaniae | aegyptius | haemoglobinophilus | haemolyticus | influenzae |
| Haemophilus | Haemophilus | Haemophilus | Haemophilus | Haemophilus |
| massiliensis | paracuniculus | parahaemolyticus | parainfluenzae | paraphrohaemolyticus |
| Haemophilus | Haemophilus | Haemophilus | Haemophilus | Haemophilus |
| pittmaniae | sp. C1 | sp. CCUG 60358 | sp. CCUG 66565 | sp. HMSC061E01 |
| Haemophilus | Haemophilus | Haemophilus | Haemophilus | Haemophilus |
| sp. HMSC066D02 | sp. HMSC066D03 | sp. HMSC068C11 | sp. HMSC073C03 | sp. HMSC61B11 |
| Haemophilus | Haemophilus | Haemophilus | Hafnia | Hafnia |
| sp. HMSC71H05 | sp. oral | sputorum | alvei | paralvei |
| | taxon 851 | | | |
| Hafnia | Hahella | Hahella | Hahella | Haladaptatus |
| sp. HMSC23F03 | chejuensis | ganghwensis | sp. CCB-MM4 | cibarius |
| Haladaptatus | Haladaptatus | Haladaptatus | Haladaptatus | Halalkalibacillus |
| litoreus | paucihalophilus | sp. R4 | sp. W1 | halophilus |
| Halalkalicoccus | Halalkalicoccus | Halanaeroarchaeum | Halanaerobium | Halanaerobium |
| jeotgali | paucihalophilus | sulfurireducens | congolense | hydrogeniformans |
| Halanaerobium | Halanaerobium | Halanaerobium | Halanaerobium | Halapricum |
| kushneri | praevalens | saccharolyticum | salsuginis | salinum |
| Halarchaeum | Halarsenatibacter | Haliangium | Haliangium | Haliea |
| acidiphilum | silvermanii | ochraceum | sp. TSM_HC | salexigens |
| Halioglobus | Halioglobus | Halioglobus | Haliscomenobacter | Haloactinobacterium |
| japonicus | pacificus | sp. H100S01 | hydrossis | album |
| Haloarchaeobius | haloarchaeon | Haloarcula | Haloarcula | Haloarcula |
| iranensis | 3A1_DGR | amylolytica | argentinensis | japonica |
| Haloarcula | Haloarcula | Halobacillus | Halobacillus | Halobacillus |
| rubripromontorii | vallismortis | aidingensis | alkaliphilus | dabanensis |
| Halobacillus | Halobacillus | Halobacillus | Halobacillus | Halobacillus |
| halophilus | hunanensis | karajensis | kuroshimensis | mangrovi |
| Halobacillus | Halobacillus | Halobacillus | Halobacillus | Halobacteriovorax |
| massiliensis | salinus | sp. BAB-2008 | sp. BBL2006 | marinus |
| Halobacterium | Halobacterium | Halobacterium | Halobacterium | Halobacterium |
| hubeiense | jilantaiense | salinarum | sp. CBA1132 | sp. DL1 |
| Halobacteroides | Halobaculum | Halobellus | Halobellus | Halobiforma |
| halobius | gomorrense | clavatus | rufus | haloterrestris |
| Halobiforma | Halobiforma | Halococcus | Halococcus | Halococcus |
| lacisalsi | nitratireducens | agarilyticus | hamelinensis | morrhuae |
| Halococcus | Halococcus | Halococcus | Halococcus | Halocynthiibacter |
| saccharolyticus | salifodinae | sediminicola | thailandensis | arcticus |
| Halocynthiibacter | Halodesulfovibrio | Halodesulfovibrio | Halodesulfovibrio | Halodesulfurarchaeum |
| namhaensis | aestuarii | marinisediminis | spirochaetisodalis | formicicum |
| Haloechinothrix | Haloferax | Haloferax | Haloferax | Haloferax |
| alba | denitrificans | gibbonsii | larsenii | massiliensis |
| Haloferax | Haloferax | Haloferax | Haloferax | Haloferax |
| mediterranei | mucosum | prahovense | sp. Q22 | sp. SB29 |
| Haloferax | Haloferax | Haloferax | Haloferula | Halofilum |
| sp. SB3 | sulfurifontis | volcanii | sp. BvORR071 | ochraceum |
| Halogeometricum | Halogeometricum | Halogeometricum | Halogeometricum | Haloglycomyces |
| borinquense | limi | pallidum | rufum | albus |
| Halogranum | Halogranum | Halogranum | Halogranum | Halohasta |
| amylolyticum | gelatinilyticum | rubrum | salarium | litchfieldiae |
| Halolactibacillus | Halolactibacillus | Halolactibacillus | Halolamina | Halolamina |
| alkaliphilus | halophilus | miurensis | pelagica | rubra |
| Halolamina | Halolamina | Halomicrobium | Halomicrobium | Halomicrobium |
| sediminis | sp. CBA1230 | katesii | mukohataei | zhouii |
| Halomicronema | Halomonas | Halomonas | Halomonas | Halomonas |
| hongdechloris | aestuarii | alkaliantarctica | anticariensis | aquamarina |
| Halomonas | Halomonas | Halomonas | Halomonas | Halomonas |
| arcis | axialensis | boliviensis | campaniensis | caseinilytica |
| Halomonas | Halomonas | Halomonas | Halomonas | Halomonas |
| chromatireducens | cupida | daqiaonensis | desiderata | elongata |
| Halomonas | Halomonas | Halomonas | Halomonas | Halomonas |
| gudaonensis | halocynthiae | halodenitrificans | huangheensis | hydrothermalis |
| Halomonas | Halomonas | Halomonas | Halomonas | Halomonas |
| ilicicola | jeotgali | korlensis | lionensis | lutea |
| Halomonas | Halomonas | Halomonas | Halomonas | Halomonas |
| meridiana | muralis | pantelleriensis | saccharevitans | salina |
| Halomonas | Halomonas | Halomonas | Halomonas | Halomonas |
| shengliensis | sinaiensis | smyrnensis | sp. 1513 | sp. ALS9 |
| Halomonas | Halomonas | Halomonas | Halomonas | Halomonas |
| sp. BC04 | sp. BC1 | sp. BC2 | sp. CSM-2 | sp. DSM 26667 |
| Halomonas | Halomonas | Halomonas | Halomonas | Halomonas |
| sp. G11 | sp. GFAJ-1 | sp. GT | sp. HAL1 | sp. HG01 |
| Halomonas | Halomonas | Halomonas | Halomonas | Halomonas |
| sp. HL-48 | sp. HL-93 | sp. JB380 | sp. KHS3 | sp. KM-1 |
| Halomonas | Halomonas | Halomonas | Halomonas | Halomonas |
| sp. KO116 | sp. KX33721 | sp. Marseille-P2426 | sp. N3-2A | sp. PBN3 |
| Halomonas | Halomonas | Halomonas | Halomonas | Halomonas |
| sp. PR-M31 | sp. QHL1 | sp. S2151 | sp. ‘Soap | sp. TD01 |
| | | | Lake #7’ | |
| Halomonas | Halomonas | Halomonas | Halomonas | Halomonas |
| stevensii | subglaciescola | subterranea | taeanensis | utahensis |
| Halomonas | Halomonas | Halomonas | Halomonas | Halonatronum |
| xianhensis | xinjiangensis | zhanjiangensis | zincidurans | saccharophilum |
| Haloparvum | Halopelagius | Halopelagius | Halopenitus | Halopenitus |
| sedimenti | inordinatus | longus | malekzadehii | persicus |
| halophilic archaeon | Halopiger | Halopiger | Halopiger | Halopiger |
| DL31 | djelfimassiliensis | goleimassiliensis | salifodinae | xanaduensis |
| Haloplanus | Haloplanus | Haloplasma | Haloprofundus | Haloquadratum |
| natans | vescus | contractile | marisrubri | walsbyi |
| Halorhabdus | Halorhabdus | Halorhodospira | Halorientalis | Halorientalis |
| tiamatea | utahensis | halophila | persicus | regularis |
| Halorientalis | Halorubrum | Halorubrum | Halorubrum | Halorubrum |
| sp. IM1011 | aidingense | coriense | distributum | ezzemoulense |
| Halorubrum | Halorubrum | Halorubrum | Halorubrum | Halorubrum |
| halodurans | hochstenium | kocurii | lacusprofiindi | lipolyticum |
| Halorubrum | Halorubrum | Halorubrum | Halorubrum | Halorubrum |
| saccharovorum | sodomense | sp. AJ67 | sp. BV1 | sp. Ea8 |
| Halorubrum | Halorubrum | Halorubrum | Halorubrum | Halorubrum |
| sp. Hd13 | sp. SAH-A6 | sp. SD612 | sp. SD626R | sp. T3 |
| Halorubrum | Halorubrum | Halorubrum | Halorubrum | Halosimplex |
| tebeniiuichense | terrestre | tropicale | vacuolatum | carlsbadense |
| Halostagnicola | Halostagnicola | Halostagnicola | Halotalea | Haloterrigena |
| kamekurae | larsenii | sp. A56 | alkalilenta | daqingensis |
| Haloterrigena | Haloterrigena | Haloterrigena | Haloterrigena | Haloterrigena |
| hispanica | jeotgali | limicola | mahii | saccharevitans |
| Haloterrigena | Haloterrigena | Haloterrigena | Halothece | Halothermothrix |
| salina | thermotolerans | turkmenica | sp. PCC 7418 | orenii |
| Halothiobacillus | Halothiobacillus | Halovenus | Hamadaea | Hammondia |
| neapolitanus | sp. LS2 | aranensis | tsunoensis | hammondi |
| Hathewaya | Helcobacillus | Helcococcus | Helcococcus | Helicobacter |
| proteolytica | massiliensis | kunzii | sueciensis | ailurogastricus |
| Helicobacter | Helicobacter | Helicobacter | Helicobacter | Helicobacter |
| apodemus | bilis | bizzozeronii | canadensis | canis |
| Helicobacter | Helicobacter | Helicobacter | Helicobacter | Helicobacter |
| cetorum | cinaedi | felis | fennelliae | heilmannii |
| Helicobacter | Helicobacter | Helicobacter | Helicobacter | Helicobacter |
| himalayensis | jaachi | japonicus | macacae | magdeburgensis |
| Helicobacter | Helicobacter | Helicobacter | Helicobacter | Helicobacter |
| muridarum | mustelae | pametensis | pullorum | pylori |
| Helicobacter | Helicobacter | Helicobacter | Helicobacter | Helicobacter |
| rodentium | saguini | sp. 11S02596-1 | sp. 11S02629-2 | sp. 11S03491-1 |
| Helicobacter | Helicobacter | Helicobacter | Helicobacter | Helicobacter |
| sp. 12S02232-10 | sp. 12S02634-8 | sp. 13S00401-1 | sp. 13S00477-4 | sp. 13S00482-2 |
| Helicobacter | Helicobacter | Helicobacter | Helicobacter | Helicobacter |
| sp. CLO-3 | sp. MIT | sp. MIT | suis | trogontum |
| | 05-5293 | 11-5569 | | |
| Helicobacter | Heliobacterium | Hellea | Henriciella | Henriciella |
| winghamensis | modesticaldum | balneolensis | aquimarina | litoralis |
| Henriciella | Henriciella | Herbaspirillum | Herbaspirillum | Herbaspirillum |
| marina | pelagia | aquaticum | autotrophicum | chlorophenolicum |
| Herbaspirillum | Herbaspirillum | Herbaspirillum | Herbaspirillum | Herbaspirillum |
| frisingense | hiltneri | huttiense | lusitanum | rhizosphaerae |
| Herbaspirillum | Herbaspirillum | Herbaspirillum | Herbaspirillum | Herbaspirillum |
| rubrisubalbicans | seropedicae | sp. B39 | sp. B65 | sp. CF444 |
| Herbaspirillum | Herbaspirillum | Herbaspirillum | Herbaspirillum | Herbaspirillum |
| sp. GW103 | sp. HZ10 | sp. meg3 | sp. RV1423 | sp. VT-16-41 |
| Herbaspirillum | Herbaspirillum | Herbiconiux | Herbiconiux | Herbiconiux |
| sp. WT00C | sp. YR522 | ginsengi | solani | sp. YR403 |
| Herbidospora | Herbidospora | Herbidospora | Herbidospora | Herbidospora |
| cretacea | daliensis | mongoliensis | sakaeratensis | yilanensis |
| Herbinix | Herminiimonas | Herminiimonas | Herminiimonas | Herpetosiphon |
| luporum | arsenicoxydans | arsenitoxidans | sp. CN | geysericola |
| Hespellia | Heterobasidion | Hippea | Hippea | Hippea |
| stercorisuis | irregulare | alviniae | jasoniae | maritima |
| Hippea | Hirschia | Hirschia | Histophilus | Histoplasma |
| sp. KM1 | baltica | maritima | somni | capsulation |
| Hoeflea | Hoeflea | Hoeflea | Hoeflea | Hoeflea |
| olei | phototrophica | sp. 108 | sp. BAL378 | sp. 1MCC20628 |
| Holdemanella | Holdemania | Holdemania | Holdemania | Holophaga |
| biformis | flliformis | massiliensis | sp. Marseille-P2844 | foetida |
| Holospora | Holospora | Hoyosella | Hoyosella | Hubei |
| obtusa | undulata | altamirensis | subflava | picorna-like |
| | | | | virus 44 |
| Hugenholtzia | Human | Human | Human | Humibacillus |
| roseola | endogenous | fecal virus | mastadenovirus C | sp. DSM |
| | retrovirus K | Jorvi2 | | 29435 |
| Humibacter | Hungatella | Hyalangium | Hydrobacter | Hydrocarboniphaga |
| albus | hathewayi | minutum | penzbergensis | daqingensis |
| Hydrocarboniphaga | Hydrococcus | Hydrocoleum | Hydrogenibacillus | Hydrogenimonas |
| effusa | rivularis | sp. CS-953 | schlegelii | thermophila |
| Hydrogenivirga | Hydrogenoanaerobacterium | Hydrogenobacter | Hydrogenobaculum | Hydrogenobaculum |
| sp. 128-5-R1-1 | saccharovorans | thermophilus | sp. HO | sp. Y04AAS1 |
| Hydrogenophaga | Hydrogenophaga | Hydrogenophaga | Hydrogenophaga | Hydrogenophaga |
| flava | intermedia | palleronii | pseudoflava | sp. A37 |
| Hydrogenophaga | Hydrogenophaga | Hydrogenophaga | Hydrogenophaga | Hydrogenophaga |
| sp. H7 | sp. 1BVHS1 | sp. 1BVHS2 | sp. LPB0072 | sp. PML113 |
| Hydrogenophaga | Hydrogenophaga | Hydrogenophaga | Hydrogenovibrio | Hydrotalea |
| sp. RAC07 | sp. Root209 | taeniospiralis | marinus | flava |
| Hylemonella | Hymenobacter | Hymenobacter | Hymenobacter | Hymenobacter |
| gracilis | actinosclerus | aerophilus | coccineus | daecheongensis |
| Hymenobacter | Hymenobacter | Hymenobacter | Hymenobacter | Hymenobacter |
| gelipurpurascens | glacialis | lapidarius | mucosus | norwichensis |
| Hymenobacter | Hymenobacter | Hymenobacter | Hymenobacter | Hymenobacter |
| psychrophilus | psychrotolerans | roseosalivarius | sedentarius | sp. APR13 |
| Hymenobacter | Hymenobacter | Hymenobacter | Hymenobacter | Hymenobacter |
| sp. CCM 8682 | sp. CRA2 | sp. DG25A | sp. DG25B | sp. IS2118 |
| Hymenobacter | Hymenobacter | Hymenobacter | Hymenobacter | Hymenobacter |
| sp. MIMBbqt21 | sp. PAMC 26554 | sp. PAMC 26628 | swuensis | terrenus |
| Hyperthermus | Hyphomicrobium | Hyphomicrobium | Hyphomicrobium | Hyphomicrobium |
| butylicus | denitrificans | facile | nitrativorans | sp. 99 |
| Hyphomicrobium | Hyphomicrobium | Hyphomicrobium | Hyphomicrobium | Hyphomicrobium |
| sp. CS1BSMeth3 | sp. CS1GBMeth3 | sp. MC1 | sp. NDB2Meth4 | sulfonivorans |
| Hyphomicrobium | Hyphomonas | Hyphomonas | Hyphomonas | Hyphomonas |
| zavarzinii | adhaerens | atlantica | beringensis | chukchiensis |
| Hyphomonas | Hyphomonas | Hyphomonas | Hyphomonas | Hyphomonas |
| jannaschiana | johnsonii | oceanitis | polymorpha | sp. L-53-1-40 |
| Hyphomonas | Hyphomonas | Hyphopichia | Hyunsoonleella | Ichthyophthirius |
| sp. Mor2 | sp. T16B2 | burtonii | jejuensis | multifiliis |
| Ideonella | Ideonella | Ideonella | Idiomarina | Idiomarina |
| sakaiensis | sp. A 288 | sp. B508-1 | abyssalis | atlantica |
| Idiomarina | Idiomarina | Idiomarina | Idiomarina | Idiomarina |
| baltica | donghaiensis | indica | loihiensis | maritima |
| Idiomarina | Idiomarina | Idiomarina | Idiomarina | Idiomarina |
| piscisalsi | planktonica | salinamm | sediminum | sp. 28-8 |
| Idiomarina | Idiomarina | Idiomarina | Idiomarina | Idiomarinaceae |
| sp. 5.13 | sp. A28L | xiamenensis | zobellii | bacterium HL-53 |
| Ignatzschineria | Ignatzschineria | Ignavibacterium | Ignavigranum | Ignicoccus |
| larvae | sp. F8392 | album | ruoffiae | hospitalis |
| Ileibacterium | Ilumatobacter | Ilumatobacter | Ilyobacter | Imhoffiella |
| massiliense | coccineus | nonamiensis | polytropus | purpurea |
| Immundisolibacter | Imtechella | Indibacter | Inediibacterium | Inquilinus |
| cernigliae | halotolerans | alkaliphilus | massiliense | limosus |
| Insolitispirillum | Intestinibacillus | Intestinibacter | Intestinimonas | Intestinimonas |
| peregrinum | massiliensis | bartlettii | butyriciproducens | massiliensis |
| Intrasporangiaceae | Intrasporangium | Intrasporangium | Intrasporangium | Isaria |
| bacterium URHB0013 | calvum | chromatireducens | oryzae | fumosorosea |
| Isobaculum | Isoptericola | Isoptericola | Isosphaera | Italian clover phyllody |
| melis | dokdonensis | variabilis | pallida | phytoplasma |
| Izhakiella | Izhakiella | Janibacter | Janibacter | Janibacter |
| capsodis | sp. D4N98 | anophelis | corallicola | hoylei |
| Janibacter | Janibacter | Janibacter | Janibacter | Janibacter |
| indicus | limosus | melonis | sp. HTCC2649 | sp. Soil728 |
| Janibacter | Jannaschia | Jannaschia | Jannaschia | Jannaschia |
| terrae | aquimarina | donghaensis | faecimaris | helgolandensis |
| Jannaschia | Jannaschia | Jannaschia | Jannaschia | Jannaschia |
| pohangensis | rubra | seosinensis | sp. CCS1 | sp. EhC01 |
| Janthinobacterium | Janthinobacterium | Janthinobacterium | Janthinobacterium | Janthinobacterium |
| agaricidamnosum | lividum | sp. 1_2014MBL_MicDiv | sp. Ant5-2-l | sp. B9-8 |
| Janthinobacterium | Janthinobacterium | Janthinobacterium | Janthinobacterium | Janthinobacterium |
| sp. CG23_2 | sp. CG3 | sp. GW458P | sp. HH01 | sp. HH106 |
| Janthinobacterium | Janthinobacterium | Janthinobacterium | Janthinobacterium | Janthinobacterium |
| sp. HH107 | sp. KBS0711 | sp. LM6 | sp. Marseille | sp. MP5059B |
| Janthinobacterium | Janthinobacterium | Janthinobacterium | Janthinobacterium | Janthinobacterium |
| sp. OK676 | sp. PC23-8 | sp. RA13 | sp. S3-2 | sp. TND4EL3 |
| Janthinobacterium | Jatrophihabitans | Jejuia | Jeongeupia | Jeotgalibaca |
| sp. YR213 | endophyticus | pallidilutea | sp. USM3 | dankookensis |
| Jeotgalibaca sp. | Jeotgalibacillus | Jeotgalibacillus | Jeotgalibacillus | Jeotgalibacillus |
| PTS2502 | alimentarius | campisalis | malaysiensis | soli |
| | | | | Cunha et al. 2012 |
| Jeotgalicoccus | Jeotgalicoccus | Jeotgalicoccus | Jeotgalicoccus | Jiangella |
| halophilus | marinus | psychrophilus | saudimassiliensis | alba |
| Jiangella | Jiangella | Jiangella | Jiangella | Jiulongibacter |
| alkaliphila | gansuensis | muralis | sp. DSM 45060 | sediminis |
| Johnsonella | Jonesia | Jonesia | Jonquetella | Joostella |
| ignava | denitrificans | quinghaiensis | anthropi | marina |
| Kaistia | Kaistia | Kaistia | Kallipyga | Kallipyga |
| adipata | granuli | soli | gabonensis | massiliensis |
| Kalmanozynui | Kamptonema | Kandleria | Kangiella | Kangiella |
| brasiliensis | formosum | vitulina | aquimarina | geojedonensis |
| Kangiella | Kangiella | Kazachstania | Kerstersia | Ketogulonicigenium |
| koreensis | sediminilitoris | africana | gyiorum | robustum |
| Ketogulonicigenium | Kibdelosporangium | Kibdelosporangium | Kibdelosporangium | Kiloniella |
| vulgare | aridum | phytohabitans | sp. MJ126-NF4 | laminariae |
| Kiloniella | Kiloniella | Kiloniella | Kineococcus | Kineosphaera |
| litopenaei | majae | spongiae | radiotolerans | limosa |
| Kineosporia | Kineosporia | Kinetoplastibacterium | Kingella | Kingella |
| sp. A_224 | sp. R_H_3 | blastocrithidii | denitrificans | kingae |
| Kingella | Kingella | Kiritimatiella | Kitasatospora | Kitasatospora |
| negevensis | oralis | glycovorans | albolonga | aureofaciens |
| Kitasatospora | Kitasatospora | Kitasatospora | Kitasatospora | Kitasatospora |
| azatica | cheerisanensis | griseola | mediocidica | phosalacinea |
| Kitasatospora | Kitasatospora | Kitasatospora | Kitasatospora | Kitasatospora |
| purpeofiisca | setae | sp. CB01950 | sp. MBT63 | sp. MBT66 |
| Kitasatospora | Kitasatospora | Kitasatospora | Klebsiella | Klebsiella |
| sp. MY 5-36 | sp. NRRL B-11411 | sp. Rootl07 | aerogenes | cf. planticola B43 |
| Klebsiella | Klebsiella | Klebsiella phage | Klebsiella | Klebsiella |
| michiganensis | oxytoca | vB_KpnM_KpV477 | pneumoniae | quasipneumoniae |
| Klebsiella | Klebsiella | Klebsiella | Klebsiella | Klebsiella |
| sp. 10982 | sp. 4_1_44FAA | sp. D5A | sp. HMSC09D12 | sp. HMSC16A12 |
| Klebsiella | Klebsiella | Klebsiella | Klebsiella | Klebsiella |
| sp. HMSC16C06 | sp. KGM- | sp. KPN1705 | sp. KTE92 | sp. LTGPAF-6F |
| | IMP216 | | | |
| Klebsiella | Klebsiella | Klebsiella | Klebsiella | Klebsiella |
| sp. MS 92-3 | sp. NFIX12 | sp. OBRC7 | sp. PO552 | sp. RIT-PI-d |
| Klebsiella | Klebsiella virus | Klebsiella virus | Kluyvera | Kluyvera |
| variicola | KLPN1 | PKP126 | ascorbata | cryocrescens |
| Kluyvera | Kluyvera | Kluyveromyces | Knoellia | Knoellia |
| georgiana | intermedia | lactis | aerolata | flava |
| Knoellia | Knoellia | Knoellia | Kockovaella | Kocuria |
| sinensis | sp. Soil729 | subterranea | imperatae | flava |
| Kocuria | Kocuria | Kocuria | Kocuria | Kocuria |
| indica | kristinae | marina | palustris | polaris |
| Kocuria | Kocuria | Kocuria | Kocuria | Kocuria |
| rhizophila | salsicia | sp. HMSC066H03 | sp. ICS0012 | sp. SM24M-10 |
| Kocuria | Kocuria | Kocuria | Kocuria | Komagataeibacter |
| sp. UCD- | sp. ZOR0020 | turfanensis | varians | europaeus |
| OTCP | | | | |
| Komagataeibacter | Komagataeibacter | Komagataeibacter | Komagataeibacter | Komagataeibacter |
| hansenii | intermedius | kakiaceti | medellinensis | nataicola |
| Komagataeibacter | Komagataeibacter | Komagataeibacter | Komagataella | Kordia |
| oboediens | rhaeticus | xylinus | phaffii | algicida |
| Kordia | Kordia | Kordiimonas | Kordiimonas | Kordiimonas |
| jejudonensis | zhangzhouensis | gwangyangensis | lacus | lipolytica |
| Kosakonia | Kosakonia | Kosakonia | Kosakonia | Kosakonia |
| arachidis | cowanii | oryzae | oryzendophytica | oryziphila |
| Kosakonia | Kosakonia | Kosakonia | Kosakonia | Kosmotoga |
| pseudosacchari | radicincitans | sacchari | sp. S29 | arenicorallina |
| Kosmotoga | Kosmotoga | Kosmotoga | Kozakia | Krasilnikoviella |
| olearia | pacifica | sp. DU53 | baliensis | flava |
| Kribbella | Kribbella | Kribbella | Kribbia | Kriegella |
| catacumbae | flavida | sp. ALI-6-A | dieselivorans | aquimaris |
| Kroppenstedtia | Ktedonobacter | Kurthia | Kurthia | Kurthia |
| eburnea | racemifer | gibsonii | huakuii | massiliensis |
| Kurthia | Kurthia | Kurthia | Kushneria | Kushneria |
| senegalensis | sp. 11kri321 | sp. Dielmo | aurantia | avicenniae |
| Kushneria | Kushneria | Kushneria | Kutzneria | Kutzneria |
| marisflavi | sp. X49 | sp. YCWA18 | albida | sp. 744 |
| Kwoniella | Kwoniella | Kwoniella | Kwoniella | Kyrpidia |
| bestiolae | dejecticola | mangrovensis | pini | tusciae |
| Kytococcus | Kytococcus | Kytococcus | Kytococcus | Labilibacter |
| aerolatus | sedentarius | sp. CUA-901 | sp. HMSC28H12 | aurantiacus |
| Labilibacter | Labrenzia | Labrenzia | Labrenzia | Labrenzia |
| marinus | aggregata | alba | alexandrii | sp. CP4 |
| Labrenzia | Labrenzia | Labrenzia | Labrenzia | Labrys |
| sp. DG1229 | sp. OB1 | sp. VG12 | suaedae | sp. WJW |
| Laccaria bicolor | Lachancea | Lachnoanaerobaculum | Lachnoanaerobaculum | Lachnoanaerobaculum |
| | thermotolerans | saburreum | sp. ICM7 | sp. MSX33 |
| Lachnoanaerobaculum | Lachnobacterium | Lachnoclostridium | Lachnoclostridium | Lachnoclostridium |
| sp. OBRC5-5 | bovis | phocaeense | phytofermentans | sp. An118 |
| Lachnoclostridium | Lachnoclostridium | Lachnoclostridium | Lachnoclostridium | Lachnoclostridium |
| sp. An131 | sp. An138 | sp. An14 | sp. An169 | sp. An181 |
| Lachnoclostridium | Lachnoclostridium | Lachnoclostridium | Lachnoclostridium | Lachnospira |
| sp. An196 | sp. An298 | sp. An76 | sp. YL32 | multipara |
| Lachnospira | Lachnospiraceae | Lachnospiraceae | Lachnospiraceae | Lachnospiraceae |
| pectinoschiza | bacterium | bacterium 1_1_57FAA | bacterium 1_4_56FAA | bacterium 10-1 |
| Lachnospiraceae | Lachnospiraceae | Lachnospiraceae | Lachnospiraceae | Lachnospiraceae |
| bacterium 2_1_46FAA | bacterium 2_1_58FAA | bacterium 28-4 | bacterium 3_1_46FAA | bacterium |
| | | | | 3_1_57FAA CT1 |
| Lachnospiraceae | Lachnospiraceae | Lachnospiraceae | Lachnospiraceae | Lachnospiraceae |
| bacterium 3-1 | bacterium 3-2 | bacterium 5_1_57FAA | bacterium 5_1_63FAA | bacterium 6_1_37FAA |
| Lachnospiraceae | Lachnospiraceae | Lachnospiraceae | Lachnospiraceae | Lachnospiraceae |
| bacterium 6_1_63FAA | bacterium 7_1_58FAA | bacterium 8_1_57FAA | bacterium | bacterium A10 |
| | | | 9_1_43BFAA | |
| Lachnospiraceae | Lachnospiraceae | Lachnospiraceae | Lachnospiraceae | Lachnospiraceae |
| bacterium A2 | bacterium A4 | bacterium AB2028 | bacterium AC2012 | bacterium AC2014 |
| Lachnospiraceae | Lachnospiraceae | Lachnospiraceae | Lachnospiraceae | Lachnospiraceae |
| bacterium AC2028 | bacterium AC2029 | bacterium AC2031 | bacterium AC3007 | bacterium AD3010 |
| Lachnospiraceae | Lachnospiraceae | Lachnospiraceae | Lachnospiraceae | Lachnospiraceae |
| bacterium C10 | bacterium C6A11 | bacterium C7 | bacterium COE1 | bacterium FD2005 |
| Lachnospiraceae | Lachnospiraceae | Lachnospiraceae | Lachnospiraceae | Lachnospiraceae |
| bacterium FE2018 | bacterium G11 | bacterium G41 | bacterium JC7 | bacterium KH1T2 |
| Lachnospiraceae | Lachnospiraceae | Lachnospiraceae | Lachnospiraceae | Lachnospiraceae |
| bacterium KHCPX20 | bacterium M18-1 | bacterium MA2020 | bacterium MC2017 | bacterium MD2004 |
| Lachnospiraceae | Lachnospiraceae | Lachnospiraceae | Lachnospiraceae | Lachnospiraceae |
| bacterium NC2004 | bacterium NC2008 | bacterium ND2006 | bacterium NE2001 | bacterium NK3A20 |
| Lachnospiraceae | Lachnospiraceae | Lachnospiraceae | Lachnospiraceae | Lachnospiraceae |
| bacterium NK4A136 | bacterium NK4A144 | bacterium NK4A179 | bacterium NLAE-zl- | bacterium oral taxon |
| | | | G231 | 082 |
| Lachnospiraceae | Lachnospiraceae | Lachnospiraceae | Lachnospiraceae | Lachnospiraceae |
| bacterium oral taxon | bacterium P6A3 | bacterium P6B14 | bacterium RM5 | bacterium TF01-11 |
| 500 | | | | |
| Lachnospiraceae | Lachnospiraceae | Lachnospiraceae | Lachnospiraceae | Lachnospiraceae |
| bacterium TWA4 | bacterium V9D3004 | bacterium VE202-12 | bacterium VE202-23 | bacterium XBB1006 |
| Lachnospiraceae | Lachnospiraceae | Lachnospiraceae | Lachnospiraceae | Lachnospiraceae |
| bacterium XBB2008 | bacterium XBD2001 | bacterium XPB1003 | bacterium YSB2008 | bacterium YSD2013 |
| Lachnotalea | Lacimicrobium | Lacimicrobium | Lacinutrix | Lacinutrix |
| glycerini | alkaliphilum | sp. SS2-24 | algicola | himadriensis |
| Lacinutrix | Lacinutrix | Lacinutrix | Lacinutrix | Lacticigenium |
| jangbogonensis | mariniflava | sp. 5H-3-7-4 | venerupis | naphtae |
| Lactobacillus | Lactobacillus | Lactobacillus | Lactobacillus | Lactobacillus |
| acetotolerans | acidifarinae | acidipiscis | acidophilus | agilis |
| Lactobacillus | Lactobacillus | Lactobacillus | Lactobacillus | Lactobacillus |
| algidus | alimentarius | amylolyticus | amylophilus | amylovorus |
| Lactobacillus | Lactobacillus | Lactobacillus | Lactobacillus | Lactobacillus |
| animalis | antri | apinorum | apis | apodemi |
| Lactobacillus | Lactobacillus | Lactobacillus | Lactobacillus | Lactobacillus |
| aquaticus | aviarius | backii | bifermentans | bombicola |
| Lactobacillus | Lactobacillus | Lactobacillus | Lactobacillus | Lactobacillus |
| brantae | brevis | buchneri | cacaonum | camelliae |
| Lactobacillus | Lactobacillus | Lactobacillus | Lactobacillus | Lactobacillus |
| capillatus | casei | ceti | coleohominis | collinoides |
| Lactobacillus | Lactobacillus | Lactobacillus | Lactobacillus | Lactobacillus |
| composti | concavus | coryniformis | crispatus | crustorum |
| Lactobacillus | Lactobacillus | Lactobacillus | Lactobacillus | Lactobacillus |
| curieae | curvatus | delbrueckii | dextrinicus | diolivorans |
| Lactobacillus | Lactobacillus | Lactobacillus | Lactobacillus | Lactobacillus |
| equi | equicursoris | equigenerosi | fabifermentans | farciminis |
| Lactobacillus | Lactobacillus | Lactobacillus | Lactobacillus | Lactobacillus |
| farraginis | fermentum | floricola | florum | fructivorans |
| Lactobacillus | Lactobacillus | Lactobacillus | Lactobacillus | Lactobacillus |
| frumenti | fuchuensis | futsaii | gallinarum | gasseri |
| Lactobacillus | Lactobacillus | Lactobacillus | Lactobacillus | Lactobacillus |
| gastricus | ghanensis | gigeriorum | ginsenosidimutans | gorillae |
| Lactobacillus | Lactobacillus | Lactobacillus | Lactobacillus | Lactobacillus |
| graminis | hammesii | hamsteri | harbinensis | hayakitensis |
| Lactobacillus | Lactobacillus | Lactobacillus | Lactobacillus | Lactobacillus |
| heilongjiangensis | helsingborgensis | helveticus | herbarum | hilgardii |
| Lactobacillus | Lactobacillus | Lactobacillus | Lactobacillus | Lactobacillus |
| hokkaidonensis | hominis | hordei | iners | ingluviei |
| Lactobacillus | Lactobacillus | Lactobacillus | Lactobacillus | Lactobacillus |
| intestinalis | jensenii | johnsonii | kalixensis | kefiranofaciens |
| Lactobacillus | Lactobacillus | Lactobacillus | Lactobacillus | Lactobacillus |
| kefiri | kimbladii | kimchicus | kimchiensis | kisonensis |
| Lactobacillus | Lactobacillus | Lactobacillus | Lactobacillus | Lactobacillus |
| kitasatonis | koreensis | kullabergensis | kunkeei | lindneri |
| Lactobacillus | Lactobacillus | Lactobacillus | Lactobacillus | Lactobacillus |
| malefermentans | mali | manihotivorans | mellifer | mellis |
| Lactobacillus | Lactobacillus | Lactobacillus | Lactobacillus | Lactobacillus |
| melliventris | mindensis | mixtipabuli | mucosae | murinus |
| Lactobacillus | Lactobacillus | Lactobacillus | Lactobacillus | Lactobacillus |
| nagelii | namurensis | nantensis | nasuensis | nodensis |
| Lactobacillus | Lactobacillus | Lactobacillus | Lactobacillus | Lactobacillus |
| odoratitofui | oeni | oligofermentans | oris | oryzae |
| Lactobacillus | Lactobacillus | Lactobacillus | Lactobacillus | Lactobacillus |
| otakiensis | ozensis | panis | pantheris | parabrevis |
| Lactobacillus | Lactobacillus | Lactobacillus | Lactobacillus | Lactobacillus |
| parabuchneri | paracasei | paracollinoides | parafarraginis | parakefiri |
| Lactobacillus | Lactobacillus | Lactobacillus | Lactobacillus | Lactobacillus |
| paralimentarius | paraplantarum | pasteurii | paucivorans | pentosus |
| Lactobacillus | Lactobacillus phage | Lactobacillus phage | Lactobacillus phage | Lactobacillus phage |
| perolens | A2 | CL2 | J-1 | Lc-Nu |
| Lactobacillus phage | Lactobacillus | Lactobacillus | Lactobacillus | Lactobacillus |
| PL-1 | plantarum | pobuzihii | pontis | psittaci |
| Lactobacillus | Lactobacillus | Lactobacillus | Lactobacillus | Lactobacillus |
| rapi | rennini | reuteri | rhamnosus | rogosae |
| Lactobacillus | Lactobacillus | Lactobacillus | Lactobacillus | Lactobacillus |
| rossiae | ruminis | saerimneri | sakei | salivarius |
| Lactobacillus | Lactobacillus | Lactobacillus | Lactobacillus | Lactobacillus |
| sanfranciscensis | saniviri | satsumensis | secaliphilus | selangorensis |
| Lactobacillus | Lactobacillus | Lactobacillus | Lactobacillus | Lactobacillus |
| senioris | senmaizukei | sharpeae | shenzhenensis | silagei |
| Lactobacillus | Lactobacillus | Lactobacillus | Lactobacillus | Lactobacillus |
| siliginis | similis | sp. ASF360 | sp. FMNP02 | sp. HMSC061B07 |
| Lactobacillus | Lactobacillus | Lactobacillus | Lactobacillus | Lactobacillus |
| sp. HMSC066G01 | sp. HMSC068B07 | sp. HMSC072E07 | sp. HMSC08B12 | sp. HMSC24D01 |
| Lactobacillus | Lactobacillus | Lactobacillus | Lactobacillus | Lactobacillus |
| sp. IWT140 | sp. IWT25 | sp. Marseille-P3519 | sp. WiKim39 | sp. wkB10 |
| Lactobacillus | Lactobacillus | Lactobacillus | Lactobacillus | Lactobacillus |
| sp. wkB8 | spicheri | sucicola | suebicus | sunkii |
| Lactobacillus | Lactobacillus | Lactobacillus | Lactobacillus | Lactobacillus |
| taiwanensis | thailandensis | tucceti | ultunensis | uvarum |
| Lactobacillus | Lactobacillus | Lactobacillus | Lactobacillus | Lactobacillus virus |
| vaccinostercus | vaginalis | versmoldensis | vini | Ld25A |
| Lactobacillus | Lactobacillus | Lactobacillus | Lactobacillus | Lactobacillus |
| virus LLKu | wasatchensis | xiangfangensis | zeae | zymae |
| Lactococcus | Lactococcus | Lactococcus | Lactococcus | Lactococcus |
| chungangensis | garvieae | lactis | phage 340 | phage BM13 |
| Lactococcus | Lactococcus | Lactococcus | Lactococcus | Lactococcus |
| phage D4410 | phage D4412 | phage jm2 | phage jm3 | phage M5938 |
| Lactococcus | Lactococcus | Lactococcus | Lactococcus | Lactococcus |
| phage M6165 | phage P680 | phage phi7 | phage Tuc2009 | phage ul36 |
| Lactococcus | Lactococcus | Lactococcus | Lactococcus | Lactococcus |
| phage WRP3 | piscium | plantarum | raffinolactis | sp. 159469 |
| Lactococcus | Lactococcus | Lactococcus | Lactococcus | Lactococcus |
| sp. DD01 | sp. Rs- | virus 712 | virus ASCC191 | virus Bibb29 |
| | Y01 | | | |
| Lactococcus | Lactococcus | Lactococcus | Lactococcus | Lactococcus |
| virus bIL170 | virus bIL67 | virus c2 | virus CB13 | virus CB14 |
| Lactococcus | Lactococcus | Lactococcus | Lactococcus | Lactonifactor |
| virus jj50 | virus P008 | virus sk1 | virus Sl4 | longoviformis |
| Lacunisphaera | Lagierella | Lamprocystis | Lampropedia | Lampropedia |
| limnophila | massiliensis | purpurea | cohaerens | hyalina |
| Laribacter | Lascolabacillus | Lausannevirus | Lautropia | Lawsonella |
| hongkongensis | massiliensis | | mirabilis | clevelandensis |
| Lawsoma | Leadbetterella | Lebetimonas | Lebetimonas | Lechevalieria |
| intracellularis | byssophila | sp. JH292 | sp. JS032 | aerocolonigenes |
| Lechevalieria | Lechevalieria | Leclercia | Leeia | Leeuwenhoekiella |
| fradiae | xinjiangensis | adecarboxylata | oryzae | blandensis |
| Leeuwenhoekiella | Leeuwenhoekiella | Leeuwenhoekiella | Leeuwenhoekiella | Legionella |
| marinoflava | palythoae | sp. Hel_1_48 | sp. MAR_2009_132 | adelaidensis |
| Legionella | Legionella | Legionella | Legionella | Legionella |
| anisa | birminghamensis | brunensis | cherrii | cincinnatiensis |
| Legionella | Legionella | Legionella | Legionella | Legionella |
| clemsonensis | drancourtii | drozanskii | erythra | fairfieldensis |
| Legionella | Legionella | Legionella | Legionella | Legionella |
| fallonii | feeleii | geestiana | hackeliae | israelensis |
| Legionella | Legionella | Legionella | Legionella | Legionella |
| jamestowniensis | jordanis | lansingensis | londiniensis | longbeachae |
| Legionella | Legionella | Legionella | Legionella | Legionella |
| maceachernii | massiliensis | moravica | nautarum | norrlandica |
| Legionella | Legionella | Legionella | Legionella | Legionella |
| oakridgensis | parisiensis | pneumophila | quateirensis | quinlivanii |
| Legionella | Legionella | Legionella | Legionella | Legionella |
| rubrilucens | sainthelensi | santicrucis | saoudiensis | shakespearei |
| Legionella | Legionella | Legionella | Legionella | Legionella |
| spiritensis | steelei | steigerwaltii | tucsonensis | tunisiensis |
| Legionella | Legionella | Legionella | Leifsonia | Leifsonia |
| wadsworthii | waltersii | worsleiensis | aquatica | rubra |
| Leifsonia | Leifsonia | Leifsonia | Leifsonia | Leifsonia |
| sp. 109 | sp. 21MFCrub1.1 | sp. AL1-44-B | sp. Leaf264 | sp. Leaf325 |
| Leifsonia | Leifsonia | Leifsonia | Leifsonia | Leifsonia |
| sp. Leaf336 | sp. NCR5 | sp. Root112D2 | sp. Root227 | sp. Root4 |
| Leifsonia | Leishmania | Leishmania | Leishmania | Leishmania |
| xyli | braziliensis | donovani | infantum | major |
| Leishmania | Leishmania | Leisingera | Leisingera | Leisingera |
| mexicana | panamensis | aquaemixtae | aquimarina | caerulea |
| Leisingera | Leisingera | Leisingera | Leisingera | Leisingera |
| daeponensis | methylohalidivorans | sp. ANG-DT | sp. ANG-M1 | sp. ANG-M7 |
| Leisingera | Leisingera | Leisingera | Lelliottia | Lelliottia |
| sp. ANG-S5 | sp. ANG-Vp | sp. JC1 | amnigena | sp. PFL01 |
| Leminorella | Lentibacillus | Lentibacillus | Lentibacillus | Lentibacillus |
| grimontii | amyloliquefaciens | halodurans | jeotgali | persicus |
| Lentibacter | Lentimicrobium | Lentisphaera | Lentzea | Lentzea |
| algarum | saccharophilum | araneosa | albida | albidocapillata |
| Lentzea | Lentzea | Lentzea | Lentzea | Lentzea |
| flaviverrucosa | guizhouensis | jiangxiensis | kentuckyensis | violacea |
| Lentzea | Leptolinea | Leptolyngbya | Leptolyngbya | Leptolyngbya |
| waywayandensis | tardivitalis | boryana | ohadii | sp. ‘hensonii’ |
| Leptolyngbya | Leptolyngbya | Leptolyngbya | Leptolyngbya | Leptolyngbya |
| sp. Heron Island J | sp. JSC-1 | sp. KIOST-1 | sp. NIES-2104 | sp. NIES-3755 |
| Leptolyngbya | Leptolyngbya | Leptolyngbya | Leptolyngbya | Leptolyngbya |
| sp. O-77 | sp. PCC 6406 | sp. PCC 7375 | sp. PCC 7376 | valderiana |
| Leptomonas | Leptonema | Leptosphaeria | Leptospira | Leptospira |
| pyrrhocoris | illini | maculans | alexanderi | alstonii |
| Leptospira | Leptospira | Leptospira | Leptospira | Leptospira |
| biflexa | borgpetersenii | broomii | fainei | inadai |
| Leptospira | Leptospira | Leptospira | Leptospira | Leptospira |
| interrogans | kirschneri | kmetyi | licerasiae | mayottensis |
| Leptospira | Leptospira | Leptospira | Leptospira | Leptospira |
| meyeri | noguchii | santarosai | sp. B5-022 | terpstrae |
| Leptospira | Leptospira | Leptospira | Leptospira | Leptospira |
| vanthielii | weilii | wolbachii | wolffii | yanagawae |
| Leptospirillum | Leptospirillum | Leptothrix | Leptothrix | Leptotrichia |
| ferriphilum | ferrooxidans | cholodnii | ochracea | buccalis |
| Leptotrichia | Leptotrichia | Leptotrichia | Leptotrichia | Leptotrichia |
| goodfellowii | hofstadii | shahii | sp. Marseille-P3007 | sp. oral |
| | | | | taxon 212 |
| Leptotrichia | Leptotrichia | Leptotrichia | Leptotrichia | Leptotrichia |
| sp. oral | sp. oral | sp. oral | sp. oral | sp. oral |
| taxon 215 | taxon 225 | taxon 498 | taxon 847 | taxon 879 |
| Leptotrichia | Leptotrichia | Leucobacter | Leucobacter | Leucobacter |
| trevisanii | wadei | celer | chironomi | chromiiresistens |
| Leucobacter | Leucobacter | Leucobacter | Leucobacter | Leucobacter |
| komagatae | musarum | salsicius | sp. 4J7B1 | sp. 7(1) |
| Leucobacter | Leucobacter | Leucobacter | Leucobacter | Leuconostoc |
| sp. Ag1 | sp. G161 | sp. PH1c | sp. UCD-THU | carnosum |
| Leuconostoc | Leuconostoc | Leuconostoc | Leuconostoc | Leuconostoc |
| citreum | fallax | garlicum | gelidum | kimchii |
| Leuconostoc | Leuconostoc | Leuconostoc | Leuconostoc | Leuconostoc |
| lactis | mesenteroides | phage P793 | phage phiLN03 | phage phiLN04 |
| Leuconostoc | Leuconostoc | Leuconostoc | Leuconostocaceae | Leucothrix mucor |
| phage phiLN6B | phage phiLNTR2 | pseudomesenteroides | bacterium R-53105 | |
| Levilinea | Levyella | Lewinella | Lewinella | Lewinella |
| saccharolytica | massiliensis | agarilytica | cohaerens | persica |
| Lewinella | Libanicoccus | Liberibacter | Lihuaxuella | Limimonas |
| sp. 4G2 | massiliensis | crescens | thermophila | halophila |
| Limnobacter | Limnochorda | Limnohabitans | Limnohabitans | Limnohabitans |
| sp. MED105 | pilosa | planktonicus | sp. 103DPR2 | sp. 2KL-27 |
| Limnohabitans | Limnohabitans | Limnohabitans | Limnohabitans | Limnohabitans |
| sp. 2KL-3 | sp. 63ED37-2 | sp. DM1 | sp. Rim11 | sp. Rim28 |
| Limnohabitans | Limnoraphis | Limnothrix | Lishizhenia | Listeria |
| sp. Rim47 | robusta | rosea | tianjinensis | aquatica |
| Listeria | Listeria | Listeria | Listeria | Listeria |
| booriae | cornellensis | fleischmannii | floridensis | grandensis |
| Listeria | Listeria | Listeria | Listeria | Listeria |
| grayi | innocua | ivanovii | monocytogenes | newyorkensis |
| Listeria | Listeria | Listeria | Listeria | Listeria |
| riparia | rocourtiae | seeligeri | sp. ILCC801 | weihenstephanensis |
| Listeria | Listeriaceae bacterium | Litoreibacter | Litoreibacter | Litoreibacter |
| welshimeri | FSL A5-0209 | albidus | arenae | ascidiaceicola |
| Litoreibacter | Litorimicrobium | Lobosporangium | Lodderomyces | Loktanella |
| janthinus | taeanense | transversale | elongisporus | atrilutea |
| Loktanella | Loktanella | Loktanella | Loktanella | Loktanella |
| cinnabarina | fryxellensis | hongkongensis | koreensis | litorea |
| Loktanella | Loktanella | Loktanella | Loktanella | Loktanella |
| marina | pyoseonensis | rosea | salsilacus | sediminum |
| Loktanella | Loktanella | Loktanella | Loktanella | Loktanella |
| soesokkakensis | sp. 1ANDIMAR09 | sp. 3ANDIMAR09 | sp. 5RATIMAR09 | sp. DSM |
| | | | | 29012 |
| Loktanella | Loktanella | Loktanella | Longilinea | Longispora |
| sp. S4079 | tamlensis | vestfoldensis | arvoryzae | albida |
| Lonsdalea | Luminiphilus | Lunatimonas | Luteibacter | Luteibacter |
| quercina | syltensis | lonarensis | rhizovicinus | sp. 22Crub2.1 |
| Luteibacter | Luteibacter | Luteibacter | Luteibacter | Luteibacter |
| sp. 329MFSha | sp. 9133 | sp. 9135 | sp. UNC138MFCol5.1 | sp. UNCMF331Sha3.1 |
| Luteibacter | Luteibacter | Luteimonas | Luteimonas | Luteimonas |
| sp. UNCMF366Tsu5.1 | yeojuensis | abyssi | huabeiensis | mephitis |
| Luteimonas | Luteimonas | Luteimonas | Luteimonas | Liiteipulveratus |
| sp. FCS-9 | sp. J29 | sp. JM171 | tolerans | halotolerans |
| Liiteipulveratus | Luteococcus | Lutibacter | Lutibacter | Lutibacter |
| mongoliensis | japonicus | agarilyticus | flavus | maritimus |
| Lutibacter | Lutibacter | Lutibacter | Lutibaculum | Lutimaribacter |
| oricola | profundi | sp. LPB0138 | baratangense | saemankumensis |
| Lutispora | Lyngbya | Lyngbya | Lyngbya | Lysinibacillus |
| thermophila | aestuarii | confervoides | sp. PCC 8106 | boronitolerans |
| Lysinibacillus | Lysinibacillus | Lysinibacillus | Lysinibacillus | Lysinibacillus |
| contaminans | fusiformis | macroides | manganicus | massiliensis |
| Lysinibacillus | Lysinibacillus | Lysinibacillus | Lysinibacillus | Lysinibacillus |
| odysseyi | sinduriensis | sp. AR18-8 | sp. BF-4 | sp. F5 |
| Lysinibacillus | Lysinibacillus | Lysinibacillus | Lysinibacillus | Lysinibacillus |
| sp. FJAT-14222 | sp. FJAT-14745 | sp. LK3 | sp. ZYM-1 | sphaericus |
| Lysinibacillus | Lysinimicrobium | Lysinimicrobium | Lysinimicrobium | Lysinimicrobium |
| xylanilyticus | aestuarii | flavum | gelatinilyticum | iriomotense |
| Lysinimicrobium | Lysinimicrobium | Lysinimicrobium | Lysinimicrobium | Lysinimicrobium |
| luteum | mangrovi | pelophilum | rhizosphaerae | soli |
| Lysinimicrobium | Lysobacter | Lysobacter | Lysobacter | Lysobacter |
| subtropicum | antibioticus | arseniciresistens | capsici | concretionis |
| Lysobacter | Lysobacter | Lysobacter | Lysobacter | Lysobacter |
| daejeonensis | defluvii | dokdonensis | enzymogenes | gummosus |
| Lysobacter | Lysobacter | Lysobacter | Lysobacter | Lysobacter |
| sp. A03 | sp. cf310 | sp. Root494 | sp. Root604 | sp. Root667 |
| Lysobacter | Lysobacter | Lysobacter | Lysobacter | Lysobacter |
| sp. Root690 | sp. Root916 | sp. Root983 | sp. URHA0019 | sp. yr284 |
| Lysobacter | Macellibacteroides | Macrococcus | Macrococcus | Mageeibacillus |
| spongiicola | sp. HH-ZS | canis | caseolyticus | indolicus |
| Magnaporthe | Magnetococcus | Magnetofaba | Magnetospira | Magnetospirillum |
| oryzae | marinus | australis | sp. QH-2 | caucaseum |
| Magnetospirillum | Magnetospirillum | Magnetospirillum | Magnetospirillum | Magnetospirillum |
| gryphiswaldense | magneticum | magnetotacticum | marisnigri | moscoviense |
| Magnetospirillum | Magnetospirillum | Magnetovibrio | Mahella | Mailhella |
| sp. ME-1 | sp. XM-1 | blakemorei | australiensis | massiliensis |
| Malassezia | Malassezia | Malassezia | Maledivibacter | Maliponia |
| globosa | pachydermatis | sympodialis | halophilus | aquimaris |
| Malonomonas | Mameliella | Mameliella | Mameliella | Mangrovibacter |
| rubra | alba | atlantica | phaeodactyli | phragmitis |
| Mangrovibacter | Mangrovimonas | Mangrovimonas | Mangrovimonas | Mangrovimonas |
| sp. MFB070 | sp. D1 80 | sp. ST2L12 | sp. ST2L15 | sp. TPBH4 |
| Mangrovimonas | Mannheimia | Mannheimia | Mannheimia | Mannheimia |
| yunxiaonensis | granulomatis | haemolytica | massilioguelmaensis | sp. USDA-ARS-USMARC- |
| | | | | 1261 |
| Mannheimia | Maribacter | Maribacter | Maribacter | Maribacter |
| varigena | antarcticus | aquivivus | arcticus | dokdonensis |
| Maribacter | Maribacter | Maribacter | Maribacter | Maribacter |
| forsetii | orientalis | sedimenticola | sp. 1_2014MBLvMicDiv | sp. B1 |
| Maribacter | Maribacter | Maribacter | Maribacter | Maribacter |
| sp. Hel_1_7 | sp. HTCC2170 | sp. MAR_2009_60 | sp. T28 | stanieri |
| Maribacter | Maribacter | Maribius | Maribius | Maribius |
| thermophilus | ulvicola | pelagius | salinus | sp. MOLA |
| | | | | 401 |
| Maricaulis | Maricaulis | Maricaulis | Marichromatium | Marichromatium |
| maris | salignorans | sp. W15 | gracile | purpuratum |
| Marinactinospora | Marinagarivorans | marine | marine gamma | marine gamma |
| thermotolerans | algicola | actinobacterium | proteobacterium | proteobacterium |
| | | PHSC20C1 | HTCC2080 | HTCC2143 |
| marine gamma | Marinibacterium | Mariniblastus | Marinicella | Marinifilum |
| proteobacterium | profundimaris | fucicola | sp. F2 | fragile |
| HTCC2148 | | | | |
| Marinilabilia | Marinilactibacillus | Marinilactibacillus | Marinilactibacillus | Marinimicrobium |
| salmonicolor | piezotolerans | psychrotolerans | sp. 15R | agarilyticum |
| Marinimicrobium | Marininema | Marininema | Mariniphaga | Mariniradius |
| sp. LS-A18 | halotolerans | mesophilum | anaerophila | saccharolyticus |
| Marinithermus | Marinitoga | Marinitoga | Marinitoga | Marinitoga |
| hydrothermalis | hydrogenitolerans | sp. 1137 | sp. 1155 | sp. 1197 |
| Marinobacter | Marinobacter | Marinobacter | Marinobacter | Marinobacter |
| adhaerens | algicola | antarcticus | daepoensis | daqiaonensis |
| Marinobacter | Marinobacter | Marinobacter | Marinobacter | Marinobacter |
| excellens | gudaonensis | hydrocarbonoclasticus | lipolyticus | lutaoensis |
| Marinobacter | Marinobacter | Marinobacter | Marinobacter | Marinobacter |
| manganoxydans | mobilis | nanhaiticus | nitratireducens | pelagius |
| Marinobacter | Marinobacter | Marinobacter | Marinobacter | Marinobacter |
| persicus | psychrophilus | salarius | salinus | santoriniensis |
| Marinobacter | Marinobacter | Marinobacter | Marinobacter | Marinobacter |
| segnicrescens | similis | sp. AC-23 | sp. BSs20148 | sp. C18 |
| Marinobacter | Marinobacter | Marinobacter | Marinobacter | Marinobacter |
| sp. C1S70 | sp. DSM | sp. EhN04 | sp. ELB17 | sp. EN3 |
| | 26291 | | | |
| Marinobacter | Marinobacter | Marinobacter | Marinobacter | Marinobacter |
| sp. es.042 | sp. es.048 | sp. ES-1 | sp. EVN1 | sp. HL-58 |
| Marinobacter | Marinobacter | Marinobacter | Marinobacter | Marinobacter |
| sp. LQ44 | sp. MCTG268 | sp. P4B1 | sp. XI5-166B | sp. YWL01 |
| Marinobacter | Marinobacter | Marinobacter | Marinobacterium | Marinobacterium |
| subterrani | vinifirmus | zhejiangensis | aestuarii | georgiense |
| Marinobacterium | Marinobacterium | Marinobacterium | Marinobacterium | Marinobacterium |
| jannaschii | litorale | profundum | rhizophilum | sp. AK27 |
| Marinobacterium | Marinococcus | Marinococcus | Marinococcus | Marinomonas |
| stanieri | halophilus | halotolerans | luteus | aquimarina |
| Marinomonas | Marinomonas | Marinomonas | Marinomonas | Marinomonas |
| atlantica | fungiae | gallaica | mediterranea | polaris |
| Marinomonas | Marinomonas | Marinomonas | Marinomonas | Marinomonas |
| posidonica | profundimaris | sp. MED121 | sp. MWYL1 | sp. QM202 |
| Marinomonas | Marinomonas | Marinomonas | Marinomonas | Marinospirillum |
| sp. S3726 | sp. TW1 | spartinae | ushiiaiensis | alkaliphilum |
| Marinospirillum | Marinospirillum | Marinospirillum | Marinovum | Mariprofundiis |
| celere | insulare | minutulum | algicola | ferrooxydans |
| Mariprofundus | Maritalea | Maritimibacter | Maritimibacter | Marivirga |
| micogutta | myrionectae | alkaliphilus | sp. HL-12 | sericea |
| Marivirga | Marivita | Marivita | Marivita | Marmoricola |
| tractuosa | cryptomonadis | geojedonensis | hallyeonensis | aequoreus |
| Marmoricola | Marmoricola | Marmoricola | Marssonina | Martelella |
| scoriae | sp. Leaf446 | sp. URHB0036 | brunnea | endophytica |
| Martelella | Martelella | Marvinbryantia | Massilia | Massilia |
| mediterranea | sp. AD-3 | formatexigens | alkalitolerans | namucuonensis |
| Massilia | Massilia | Massilia | Massilia | Massilia |
| niastensis | putida | sp. 9096 | sp. BSC265 | sp. CF038 |
| Massilia | Massilia | Massilia | Massilia | Massilia |
| sp. JS1662 | sp. KIM | sp. LC238 | sp. Leaf139 | sp. NR 4-1 |
| Massilia | Massilia | Massilia | Massilia | Massilia |
| sp. PDC64 | sp. Root1485 | sp. Root335 | sp. Root351 | sp. Root418 |
| Massilia | Massilia | Massilia | Massilibacterium | Massilibacteroides |
| sp. WF1 | timonae | yuzhufengensis | senegalense | vaginae |
| Massilimaliae | Massilimaliae | Massilioclostridium | Massiliomicrobiota | Massiliomicrobiota |
| massiliensis | sp. Marseille-P3753 | coli | sp. An105 | sp. An134 |
| Massiliomicrobiota | Massiliomicrobiota | Massiliomicrobiota | Mastigocladopsis | Mastigocladus |
| sp. An142 | sp. An80 | timonensis | repens | laminosus |
| Mastigocoleus | Mediterranea | Mediterranea | Megamonas | Megamonas |
| testarum | massiliensis | sp. An20 | funiformis | hypermegale |
| Megamonas | Megamonas | Megamonas | Meganema | Megasphaera |
| rupellensis | sp. Calf98-2 | sp. Marseille-P3344 | perideroedes | cerevisiae |
| Megasphaera | Megasphaera | Megasphaera | Megasphaera | Megasphaera |
| elsdenii | genomosp. type_1 | massiliensis | micronuciformis | paucivorans |
| Megasphaera | Megasphaera | Megasphaera | Megasphaera | Megasphaera |
| sp. An286 | sp. BL7 | sp. BV3C16-1 | sp. DISK 18 | sp. DJF_B143 |
| Megasphaera | Megasphaera | Megasphaera | Megasphaera | Megavirus |
| sp. MJR8396C | sp. NM10 | sp. UPII | sp. UPII | chiliensis |
| | | 135-E | 199-6 | |
| Meiothermus | Meiothermus | Meiothermus | Meiothermus | Meiothermus |
| cerbereus | chliarophilus | ruber | rufus | silvanus |
| Meiothermus | Meiothermus | Melaminivora | Melampsora | Melghirimyces |
| taiwanensis | timidus | alkalimesophila | larici- | thermohalophilus |
| | | | populina | |
| Melioribacter | Melissococcus | Merdibacter | Merdimonas | Mesoaciditoga |
| roseus | plutonius | massiliensis | faecis | lauensis |
| Mesoflavibacter | Mesonia | Mesonia | Mesoplasma | Mesoplasma |
| zeaxanthinifaciens | mobilis | phycicola | chauliocola | florum |
| Mesoplasma | Mesoplasma | Mesoplasma | Mesoplasma | Mesorhizobium |
| grammopterae | photuris | seiffertii | syrphidae | alhagi |
| Mesorhizobium | Mesorhizobium | Mesorhizobium | Mesorhizobium | Mesorhizobium |
| amorphae | australicum | ciceri | erdmanii | huakuii |
| Mesorhizobium | Mesorhizobium | Mesorhizobium | Mesorhizobium | Mesorhizobium |
| loti | mediterraneum | metallidurans | muleiense | opportunistum |
| Mesorhizobium | Mesorhizobium | Mesorhizobium | Mesorhizobium | Mesorhizobium |
| plurifarium | prunaredense | qingshengii | sophorae | sp. 1M-11 |
| Mesorhizobium | Mesorhizobium | Mesorhizobium | Mesorhizobium | Mesorhizobium |
| sp. AA22 | sp. AA23 | sp. B7 | sp. F7 | sp. ICMP 18942 |
| Mesorhizobium | Mesorhizobium | Mesorhizobium | Mesorhizobium | Mesorhizobium |
| sp. L103C105A0 | sp. L103C131B0 | sp. L103C565B0 | sp. L2C054A000 | sp. L2C066B000 |
| Mesorhizobium | Mesorhizobium | Mesorhizobium | Mesorhizobium | Mesorhizobium |
| sp. L2C067A000 | sp. L2C084A000 | sp. L2C085B000 | sp. L2C089B000 | sp. L48C026A00 |
| Mesorhizobium | Mesorhizobium | Mesorhizobium | Mesorhizobium | Mesorhizobium |
| sp. LC103 | sp. LNHC209A00 | sp. LNHC220B00 | sp. LNHC221B00 | sp. LNHC229A00 |
| Mesorhizobium | Mesorhizobium | Mesorhizobium | Mesorhizobium | Mesorhizobium |
| sp. LNHC232B00 | sp. LNHC252B00 | sp. LNJC374B00 | sp. LNJC380A00 | sp. LNJC384A00 |
| Mesorhizobium | Mesorhizobium | Mesorhizobium | Mesorhizobium | Mesorhizobium |
| sp. LNJC391B00 | sp. LNJC394B00 | sp. LNJC395A00 | sp. LNJC398B00 | sp. LNJC399B00 |
| Mesorhizobium | Mesorhizobium | Mesorhizobium | Mesorhizobium | Mesorhizobium |
| sp. LNJC403B00 | sp. LNJC405B00 | sp. LSHC412B00 | sp. LSHC416B00 | sp. LSHC420B00 |
| Mesorhizobium | Mesorhizobium | Mesorhizobium | Mesorhizobium | Mesorhizobium |
| sp. LSHC422A00 | sp. LSHC424B00 | sp. LSHC432A00 | sp. LSHC440A00 | sp. LSJC255A00 |
| Mesorhizobium | Mesorhizobium | Mesorhizobium | Mesorhizobium | Mesorhizobium |
| sp. LSJC264A00 | sp. LSJC265A00 | sp. LSJC268A00 | sp. LSJC269B00 | sp. LSJC277A00 |
| Mesorhizobium | Mesorhizobium | Mesorhizobium | Mesorhizobium | Mesorhizobium |
| sp. LSJC280B00 | sp. LSJC285A00 | sp. NBIMC_P2-C2 | sp. ORS3324 | sp. ORS3359 |
| Mesorhizobium | Mesorhizobium | Mesorhizobium | Mesorhizobium | Mesorhizobium |
| sp. ORS3428 | sp. Root102 | sp. Root157 | sp. Root552 | sp. Root695 |
| Mesorhizobium | Mesorhizobium | Mesorhizobium | Mesorhizobium | Mesorhizobium |
| sp. SEMIA 3007 | sp. SOD10 | sp. STM 4661 | sp. UASWS1009 | sp. URHA0056 |
| Mesorhizobium | Mesorhizobium | Mesorhizobium | Mesorhizobium | Mesorhizobium |
| sp. URHB0007 | sp. URHC0008 | sp. WSM1293 | sp. WSM1497 | sp. WSM2561 |
| Mesorhizobium | Mesorhizobium | Mesorhizobium | Mesorhizobium | Mesorhizobium |
| sp. WSM3224 | sp. WSM3626 | sp. WSM3873 | sp. WYCCWR 10019 | sp. YR577 |
| Mesorhizobium | Mesotoga | Metallibacterium | Metallosphaera | Metarhizium |
| temperatum | prima | scheffleri | sedula | acridum |
| Metarhizium | Metarhizium | Metarhizium | Methanobacterium | Methanobacterium |
| brunneum | majus | robertsii | congolense | formicicum |
| Methanobacterium | Methanobacterium | Methanobacterium | Methanobacterium | Methanobacterium |
| lacus | paludis | sp. A39 | sp. MB1 | sp. SMA-27 |
| Methanobrevibacter | Methanobrevibacter | Methanobrevibacter | Methanobrevibacter | Methanobrevibacter |
| arboriphilus | boviskoreani | curvatus | cuticularis | filiformis |
| Methanobrevibacter | Methanobrevibacter | Methanobrevibacter | Methanobrevibacter | Methanobrevibacter |
| gottschalkii | millerae | olleyae | oralis | ruminantium |
| Methanobrevibacter | Methanobrevibacter | Methanobrevibacter | Methanobrevibacter | Methanobrevibacter |
| smithii | sp. 87.7 | sp. A27 | sp. A54 | sp. AbM4 |
| Methanobrevibacter | Methanobrevibacter | Methanocaldococcus | Methanocaldococcus | Methanocaldococcus |
| sp. YE315 | wolinii | bathoardescens | infernus | sp. FS406-22 |
| Methanocaldococcus | Methanocella | Methanocella | Methanococcoides | Methanococcoides |
| villosus | arvoryzae | conradii | burtonii | methylutens |
| Methanococcoides | Methanococcus | Methanococcus | Methanococcus | Methanococcus |
| vulcani | aeolicus | maripaludis | vannielii | voltae |
| Methanocorpusculum | Methanocorpusculum | Methanoculleus | Methanoculleus | Methanoculleus |
| bavaricum | labreanum | bourgensis | chikugoensis | horonobensis |
| Methanoculleus | Methanoculleus | Methanoculleus | Methanoculleus | Methanoculleus |
| marisnigri | sediminis | sp. MAB1 | sp. MH98A | thermophilus |
| Methanofollis | Methanofollis | methanogenic | Methanogenium | Methanohalobium |
| ethanolicus | liminatans | archaeon ISO4-H5 | cariaci | evestigatum |
| Methanohalophilus | Methanohalophilus | Methanohalophilus | Methanolacinia | Methanolacinia |
| halophilus | mahii | portucalensis | paynteri | petrolearia |
| Methanolinea | Methanolobus | Methanolobus | Methanolobus | Methanolobus |
| tarda | profundi | psychrophilus | sp. YSF-03 | tindarius |
| Methanolobus | Methanomassiliicoccus | Methanomethylovorans | Methanomicrobium | Methanoplanus |
| vulcani | luminyensis | hollandica | mobile | limicola |
| Methanopyrus | Methanoregula | Methanoregula | Methanosaeta | Methanosaeta |
| sp. KOL6 | boonei | formicica | concilii | harundinacea |
| Methanosalsum | Methanosarcina | Methanosarcina | Methanosarcina | Methanosarcina |
| zhilinae | acetivorans | barkeri | flavescens | horonobensis |
| Methanosarcina | Methanosarcina | Methanosarcina | Methanosarcina | Methanosarcina |
| lacustris | mazei | siciliae | soligelidi | sp. 1.H.T.1A.1 |
| Methanosarcina | Methanosarcina | Methanosarcina | Methanosarcina | Methanosphaera |
| sp. Kolksee | sp. MTP4 | thermophila | vacuolata | sp. WGK6 |
| Methanosphaera | Methanosphaemla | Methanospirillum | Methanothermobacter | Methanothermobacter |
| stadtmanae | palustris | hungatei | marburgensis | sp. CaT2 |
| Methanothermobacter | Methanothermobacter | Methanothermococcus | Methanothermus | Methanotorris |
| thermautotrophicus | wolfeii | okinawensis | fervidus | formicicus |
| Methanotorris | Methermicoccus | Methylacidiphilum | Methylacidiphilum | Methylacidiphilum |
| igneus | shengliensis | fumariolicum | infernorum | kamchatkense |
| Methylibium | Methylibium | Methylibium | Methylibium | Methylibium |
| petroleiphilum | sp. CF059 | sp. CF468 | sp. Root1272 | sp. YR605 |
| Methylobacillus | Methylobacillus | Methylobacillus | Methylobacillus | Methylobacter |
| flagellatus | glycogenes | rhizosphaerae | sp. MM2 | luteus |
| Methylobacter | Methylobacter | Methylobacter | Methylobacter | Methylobacterium |
| marinus | sp. BBA5.1 | tundripaludum | whittenburyi | aquaticum |
| Methylobacterium | Methylobacterium | Methylobacterium | Methylobacterium | Methylobacterium |
| brachiatum | extorquens | gossipiicola | indicium | mesophilicum |
| Methylobacterium | Methylobacterium | Methylobacterium | Methylobacterium | Methylobacterium |
| nodulans | phyllosphaerae | phyllostachyos | platani | populi |
| Methylobacterium | Methylobacterium | Methylobacterium | Methylobacterium | Methylobacterium |
| pseudosasicola | radiotolerans | salsuginis | sp. 10 | sp. 13MFTsu3.1M2 |
| Methylobacterium | Methylobacterium | Methylobacterium | Methylobacterium | Methylobacterium |
| sp. 174MFSha1.1 | sp. 275MFSha3.1 | sp. 4-46 | sp. 77 | sp. 88A |
| Methylobacterium | Methylobacterium | Methylobacterium | Methylobacterium | Methylobacterium |
| sp. AMS5 | sp. ap11 | sp. ARG-1 | sp. B1 | sp. C1 |
| Methylobacterium | Methylobacterium | Methylobacterium | Methylobacterium | Methylobacterium |
| sp. CCH5-D2 | sp. GXF4 | sp. GXS13 | sp. Leaf100 | sp. Leaf104 |
| Methylobacterium | Methylobacterium | Methylobacterium | Methylobacterium | Methylobacterium |
| sp. Leaf106 | sp. Leaf108 | sp. Leaf112 | sp. Leaf113 | sp. Leaf117 |
| Methylobacterium | Methylobacterium | Methylobacterium | Methylobacterium | Methylobacterium |
| sp. Leaf122 | sp. Leaf123 | sp. Leaf125 | sp. Leaf361 | sp. Leaf399 |
| Methylobacterium | Methylobacterium | Methylobacterium | Methylobacterium | Methylobacterium |
| sp. Leaf456 | sp. Leaf465 | sp. Leaf466 | sp. Leaf85 | sp. Leaf86 |
| Methylobacterium | Methylobacterium | Methylobacterium | Methylobacterium | Methylobacterium |
| sp. Leaf88 | sp. Leaf90 | sp. Leaf92 | sp. Leaf93 | sp. Leaf94 |
| Methylobacterium | Methylobacterium | Methylobacterium | Methylobacterium | Methylobacterium |
| sp. Leaf99 | sp. MB200 | sp. UNC378MF | sp. WSM2598 | sp. yr668 |
| Methylobacterium | Methylobacterium | Methylobacterium | Methylobrevis | Methylocaldum |
| sp. ZNC0032 | tarhaniae | variabile | pamukkalensis | sp. 14B |
| Methylocaldum | Methylocaldum | Methylocaldum | Methylocapsa | Methylocapsa |
| sp. 175 | sp. SAD2 | szegediense | acidiphila | aurea |
| Methylocapsa | Methyloceanibacter | Methyloceanibacter | Methyloceanibacter | Methyloceanibacter |
| palsamm | caenitepidi | marginalis | methanicus | stevinii |
| Methyloceanibacter | Methylocella | Methylococcaceae | Methylococcaceae | Methylococcus |
| superfactus | Silvestris | bacterium 73a | bacterium Sn10-6 | capsulatus |
| Methylocystis | Methylocystis | Methylocystis | Methylocystis | Methylocystis |
| bryophila | parvus | rosea | sp. ATCC 49242 | sp. SB2 |
| Methylocystis | Methyloferula | Methylogaea | Methyloglobulus | Methylohalobius |
| sp. SC2 | stellata | oryzae | morosus | crimeensis |
| Methyloligella | Methylomarinum | Methylomicrobium | Methylomicrobium | Methylomicrobium |
| halotolerans | vadi | album | alcaliphilum | buryatense |
| Methylomonas | Methylomonas | Methylomonas | Methylomonas | Methylomonas |
| denitrificans | koyamae | lenta | methanica | sp. 11b |
| Methylomonas | Methylomonas | Methylomonas | Methylomonas | Methylophaga |
| sp. DH-1 | sp. LW13 | sp. LWB | sp. MK1 | aminisulfidivorans |
| Methylophaga | Methylophaga | Methylophaga | Methylophaga | Methylophaga |
| frappieri | lonarensis | muralis | nitratireducenticrescens | sulfidovorans |
| Methylophaga | Methylophilales | Methylophilales | Methylophilus | Methylophilus |
| thiooxydans | bacterium HTCC2181 | bacterium LSUCC0135 | methylotrophus | rhizosphaerae |
| Methylophilus | Methylophilus | Methylophilus | Methylophilus | Methylophilus |
| sp. 5 | sp. Leaf414 | sp. OH31 | sp. Q8 | sp. TWE2 |
| Methylopila | Methylopila | Methyloprofundus | Methylosarcina | Methylosarcina |
| sp. 73B | sp. M107 | sedimenti | fibrata | lacus |
| Methylosinus | Methylosinus | Methylosinus | Methylosinus | Methylotenera |
| sp. LW3 | sp. LW4 | sp. PW1 | sp. R-45379 | mobilis |
| Methylotenera | Methylotenera | Methylotenera | Methylotenera | Methyloversatilis |
| sp. G11 | sp. L2L1 | sp. N17 | versatilis | discipulorum |
| Methyloversatilis | Methyloversatilis | Methyloversatilis | Methylovorus | Methylovorus |
| sp. RAC08 | thermotolerans | universalis | glucosotrophus | sp. MM1 |
| Methylovorus | Methylovulum | Methylovulum | Metschnikowia | Meyerozyma |
| sp. MP688 | miyakonense | psychrotolerans | bicuspidata | guilliermondii |
| Micavibrio | Microbacteriaceae | Microbacterium | Microbacterium | Microbacterium |
| aeruginosavorus | bacterium | arborescens | aurum | azadirachtae |
| Microbacterium | Microbacterium | Microbacterium | Microbacterium | Microbacterium |
| barkeri | chocolatum | enclense | esteraromaticum | foliorum |
| Microbacterium | Microbacterium | Microbacterium | Microbacterium | Microbacterium |
| ginsengisoli | gorillae | gubbeenense | hominis | humi |
| Microbacterium | Microbacterium | Microbacterium | Microbacterium | Microbacterium |
| hydrocarbonoxydans | indicum | ketosireducens | laevaniformans | luticocti |
| Microbacterium | Microbacterium | Microbacterium | Microbacterium | Microbacterium |
| mangrovi | maritypicum | oleivorans | oxydans | paludicola |
| Microbacterium | Microbacterium | Microbacterium | Microbacterium | Microbacterium |
| paraoxydans | profundi | pygmaeum | resistens | sediminis |
| Microbacterium | Microbacterium | Microbacterium | Microbacterium | Microbacterium |
| sp. 11MF | sp. 292MF | sp. 3J1 | sp. Ag1 | sp. AISO3 |
| Microbacterium | Microbacterium | Microbacterium | Microbacterium | Microbacterium |
| sp. AR7-10 | sp. B19 | sp. B24 | sp. BH-3-3-3 | sp. C448 |
| Microbacterium | Microbacterium | Microbacterium | Microbacterium | Microbacterium |
| sp. CCH5-D1 | sp. cf046 | sp. cf332 | sp. CF335 | sp. CH1 |
| Microbacterium | Microbacterium | Microbacterium | Microbacterium | Microbacterium |
| sp. CH12i | sp. cl127 | sp. cl140 | sp. Cr-K29 | sp. CSI-V |
| Microbacterium | Microbacterium | Microbacterium | Microbacterium | Microbacterium |
| sp. GCS4 | sp. H83 | sp. HM58-2 | sp. KROCY2 | sp. LCT-H2 |
| Microbacterium | Microbacterium | Microbacterium | Microbacterium | Microbacterium |
| sp. Leaf151 | sp. Leaf159 | sp. Leaf161 | sp. Leaf179 | sp. Leaf203 |
| Microbacterium | Microbacterium | Microbacterium | Microbacterium | Microbacterium |
| sp. Leaf288 | sp. Leaf320 | sp. Leaf436 | sp. LEMMJ01 | sp. LKL04 |
| Microbacterium | Microbacterium | Microbacterium | Microbacterium | Microbacterium |
| sp. MEJ108Y | sp. MRS-1 | sp. No. 7 | sp. oral taxon 186 | sp. PAMC 28756 |
| Microbacterium | Microbacterium | Microbacterium | Microbacterium | Microbacterium |
| sp. Root1433D11 | sp. Root166 | sp. Root180 | sp. Root280D1 | sp. Root322 |
| Microbacterium | Microbacterium | Microbacterium | Microbacterium | Microbacterium |
| sp. Root53 | sp. Root553 | sp. Root61 | sp. RU1D | sp. RU33B |
| Microbacterium | Microbacterium | Microbacterium | Microbacterium | Microbacterium |
| sp. ru370.1 | sp. RURRCA19A | sp. SA39 | sp. T32 | sp. TNHR37B |
| Microbacterium | Microbacterium | Microbacterium | Microbacterium | Microbacterium |
| sp. TS-1 | sp. UCD-TDU | sp. URHA0036 | sp. XT11 | sp. Yaish 1 |
| Microbacterium | Microbacterium | Microbacterium | Microbacterium | Microbispora |
| sp. ZOR0019 | testaceum | trichothecenolyticum | yannicii | rosea |
| Microbispora | Microbispora | Microbispora | Microbispora | Microbispora |
| sp. ATCC PTA-5024 | sp. GKU 823 | sp. GMKU363 | sp. NBRC 110460 | sp. NRRL B-24597 |
| Microbulbifer | Microbulbifer | Microbulbifer | Microbulbifer | Microbulbifer |
| agarilyticus | donghaiensis | mangrovi | marinas | sp. CCB-MM1 |
| Microbulbifer | Microbulbifer | Microbulbifer | Microbulbifer | Microbulbifer |
| sp. HZ11 | sp. Q7 | sp. ZGT114 | thermotolerans | variabilis |
| Microbulbifer | Micrococcales | Micrococcus | Micrococcus | Micrococcus |
| yueqingensis | bacterium KH10 | aloeverae | luteus | lylae |
| Micrococcus | Micrococcus | Micrococcus | Micrococcus | Micrococcus |
| sp. CH3 | sp. CH7 | sp. FDAARGOS_333 | sp. HMSC31B01 | sp. KBS0714 |
| Micrococcus | Micrococcus | Microcoleus | Microcoleus | Microcystis |
| sp. MS- | terreus | sp. PCC | vaginatus | aeruginosa |
| ASIII-49 | | 7113 | | |
| Microcystis | Microlunatus | Microlunatus | Micromonospora | Micromonospora |
| sp. T1-4 | phosphovorus | soli | auratinigra | avicenniae |
| Micromonospora | Micromonospora | Micromonospora | Micromonospora | Micromonospora |
| carbonacea | chaiyaphumensis | chersina | chokoriensis | citrea |
| Micromonospora | Micromonospora | Micromonospora | Micromonospora | Micromonospora |
| coriariae | coxensis | cremea | eburnea | echinaurantiaca |
| Micromonospora | Micromonospora | Micromonospora | Micromonospora | Micromonospora |
| echinofusca | echinospora | haikouensis | halophytica | humi |
| Micromonospora | Micromonospora | Micromonospora | Micromonospora | Micromonospora |
| inositola | inyonensis | krabiensis | lupini | marina |
| Micromonospora | Micromonospora | Micromonospora | Micromonospora | Micromonospora |
| matsumotoense | mirobrigensis | narathiwatensis | nigra | pallida |
| Micromonospora | Micromonospora | Micromonospora | Micromonospora | Micromonospora |
| parva | pattaloongensis | peucetia | purpureochromogenes | rhizosphaerae |
| Micromonospora | Micromonospora | Micromonospora | Micromonospora | Micromonospora |
| rifamycinica | rosaria | saelicesensis | sediminicola | siamensis |
| Micromonospora | Micromonospora | Micromonospora | Micromonospora | Micromonospora |
| sp. ATCC 39149 | sp. CB01531 | sp. CNB394 | sp. HK10 | sp. II |
| Micromonospora | Micromonospora | Micromonospora | Micromonospora | Micromonospora |
| sp. M42 | sp. NBRC 110037 | sp. NBS11-29 | sp. NRRL B-16802 | sp. Rc5 |
| Micromonospora | Micromonospora | Micromonospora | Micromonospora | Micromonospora |
| sp. RV43 | sp. TSR10369 | tulbaghiae | viridifaciens | wenchangensis |
| Micromonospora | Micromonospora | Microscilla | Microsporum | Microterricola |
| yangpuensis | zamorensis | marina | canis | viridarii |
| Microtetraspora | Microtetraspora | Microtetraspora | Microtetraspora | Microvirga |
| fusca | glauca | malaysiensis | niveoalba | flocculans |
| Microvirga | Microvirga | Microvirga | Microvirga | Microvirga |
| guangxiensis | lotononidis | massiliensis | sp. BSC39 | vignae |
| Microvirgula | Milkweed yellows | Millionella | Millisia | Mimivirus |
| aerodenitrificans | phytoplasma | massiliensis | brevis | terra2 |
| Mitosporidium | Mitsuaria | Mitsuaria | Mitsuaria | Mitsuaria |
| daphniae | chitosanitabida | sp. 7 | sp. HZ7 | sp. PDC51 |
| Mitsuokella | Mitsuokella | Mitsuokella | Mixia | Mizugakiibacter |
| jalaludinii | multacida | sp. oral | osmundae | sediminis |
| | | taxon 131 | | |
| Mobilibacterium | Mobilicoccus | Mobilicoccus | Mobiluncus | Mobiluncus |
| timonense | massiliensis | pelagius | curtisii | mulieris |
| Modestobacter | Modestobacter | Modestobacter | Modestobacter | Moellerella |
| caceresii | marinus | sp. DSM 44400 | sp. Leaf380 | wisconsensis |
| Moesziomyces | Mogibacterium | Mogibacterium | Mogibacterium | Moheibacter |
| antarcticus | pumilum | sp. CM50 | timidum | sediminis |
| Monashia | Mongoliimonas | Moniliophthora | Moorea | Moorea |
| flava | terrestris | roreri | bouillonii | producens |
| Moorella | Moorella | Moorella | Moraxella | Moraxella |
| glycerini | mulderi | thermoacetica | atlantae | boevrei |
| Moraxella | Moraxella | Moraxella | Moraxella | Moraxella |
| bovis | bovoculi | canis | caprae | catarrhalis |
| Moraxella | Moraxella | Moraxella | Moraxella | Moraxella |
| caviae | cuniculi | equi | lacunata | lincolnii |
| Moraxella | Moraxella | Moraxella | Moraxella | Moraxella |
| macacae | nonliquefaciens | oblonga | osloensis | ovis |
| Moraxella | Moraxella | Moraxella | Morganella | Morganella |
| pluranimalium | porci | sp. VT-16-12 | morganii | psychrotolerans |
| Morganella | Moritella | Moritella | Moritella | Moritella |
| sp. HMSC11D09 | dasanensis | marina | sp. JT01 | sp. PE36 |
| Moritella | Morococcus | Motiliproteus | Moumouviriis | Mucilaginibacter |
| viscosa | cerebrosus | sp. MSK22-1 | | gossypii |
| Mucilaginibacter | Mucilaginibacter | Mucilaginibacter | Mucilaginibacter | Mucilaginibacter |
| gossypiicola | lappiensis | mallensis | paludis | pedocola |
| Mucilaginibacter | Mucilaginibacter | Mucilaginibacter | Mucilaginibacter | Mucilaginibacter |
| pineti | polytrichastri | sp. BJC16-A31 | sp. L294 | sp. NFR10 |
| Mucilaginibacter | Mucilaginibacter | Mucilaginibacter | Mucilaginibacter | Mucilaginibacter |
| sp. OK098 | sp. OK268 | sp. OK283 | sp. PAMC 26640 | sp. PPCGB 2223 |
| Mucinivorans | Mucispirillum | Murdochiella | Murdochiella | Muribacter muris |
| hirudinis | schaedleri | massiliensis | sp. Marseille-P2341 | |
| Miiribaculum | Muribaculum | Muricauda | Muricauda | Muricauda |
| sp. An287 | sp. An289 | antarctica | lutaonensis | ruestringensis |
| Muricauda | Muricauda | Muricauda | Mycetocola | Mycetocola |
| sp. CP2A | sp. MAR_2010_75 | zhangzhouensis | miduiensis | reblochoni |
| Mycetocola | Mycobacterium | Mycobacterium | Mycobacterium | Mycobacterium |
| saprophilus | abscessus | acapulcensis | africanum | algericum |
| Mycobacterium | Mycobacterium | Mycobacterium | Mycobacterium | Mycobacterium |
| alsense | angelicum | aquaticum | aromaticivorans | arosiense |
| Mycobacterium | Mycobacterium | Mycobacterium | Mycobacterium | Mycobacterium |
| arupense | asiaticum | aurum | austroafricanum | avium |
| Mycobacterium | Mycobacterium | Mycobacterium | Mycobacterium | Mycobacterium |
| bacteremicum | boenickei | bohemicum | bovis | branderi |
| Mycobacterium | Mycobacterium | Mycobacterium | Mycobacterium | Mycobacterium |
| brisbanense | brumae | canariasense | canettii | celatum |
| Mycobacterium | Mycobacterium | Mycobacterium | Mycobacterium | Mycobacterium |
| celeriflavum | chelonae | chimaera | chlorophenolicum | chubuense |
| Mycobacterium | Mycobacterium | Mycobacterium | Mycobacterium | Mycobacterium |
| colombiense | conceptionense | confluentis | conspicuum | cosmeticum |
| Mycobacterium | Mycobacterium | Mycobacterium | Mycobacterium | Mycobacterium |
| diernhoferi | dioxanotrophicus | doricum | engbaekii | europaeum |
| Mycobacterium | Mycobacterium | Mycobacterium | Mycobacterium | Mycobacterium |
| fallax | farcinogenes | flavescens | florentinum | fortuitum |
| Mycobacterium | Mycobacterium | Mycobacterium | Mycobacterium | Mycobacterium |
| fragae | franklinii | gastri | genavense | gilvum |
| Mycobacterium | Mycobacterium | Mycobacterium | Mycobacterium | Mycobacterium |
| goodii | gordonae | haemophilum | hassiacum | heckeshornense |
| Mycobacterium | Mycobacterium | Mycobacterium | Mycobacterium | Mycobacterium |
| heidelbergense | heraklionense | hiberniae | holsaticum | houstonense |
| Mycobacterium | Mycobacterium | Mycobacterium | Mycobacterium | Mycobacterium |
| icosiumassiliensis | immunogenum | indicuspranii | insubricum | interjectum |
| Mycobacterium | Mycobacterium | Mycobacterium | Mycobacterium | Mycobacterium |
| intermedium | intracellulare | iranicum | kansasii | komanii |
| Mycobacterium | Mycobacterium | Mycobacterium | Mycobacterium | Mycobacterium |
| koreense | kumamotonense | kyorinense | lacus | lentiflavum |
| Mycobacterium | Mycobacterium | Mycobacterium | Mycobacterium | Mycobacterium |
| leprae | lepromatosis | litorale | llatzerense | longobardum |
| Mycobacterium | Mycobacterium | Mycobacterium | Mycobacterium | Mycobacterium |
| mageritense | malmesburyense | malmoense | mantenii | marinum |
| Mycobacterium | Mycobacterium | Mycobacterium | Mycobacterium | Mycobacterium |
| minnesotense | monacense | moriokaense | mucogenicum | nebraskense |
| Mycobacterium | Mycobacterium | Mycobacterium | Mycobacterium | Mycobacterium |
| neoaurum | neworleansense | nonchromogenicum | noviomagense | novocastrense |
| Mycobacterium | Mycobacterium | Mycobacterium | Mycobacterium | Mycobacterium |
| obuense | palustre | paraense | paraffinicum | parafortuitum |
| Mycobacterium | Mycobacterium | Mycobacterium | Mycobacterium | Mycobacterium |
| paraseoulense | parmense | peregrinum | persicum | phlei |
| Mycobacterium | Mycobacterium | Mycobacterium | Mycobacterium | Mycobacterium |
| porcinum | rhodesiae | riyadhense | rutilum | salmoniphilum |
| Mycobacterium | Mycobacterium | Mycobacterium | Mycobacterium | Mycobacterium |
| saopaulense | saskatchewanense | scrofulaceum | senegalense | senuense |
| Mycobacterium | Mycobacterium | Mycobacterium | Mycobacterium | Mycobacterium |
| septicum | setense | sherrisii | shimoidei | shinjukuense |
| Mycobacterium | Mycobacterium | Mycobacterium | Mycobacterium | Mycobacterium |
| simiae | sinense | smegmatis | sp. (ex | sp. 1081908.1 |
| | | | Dasyatis americana) | |
| Mycobacterium | Mycobacterium | Mycobacterium | Mycobacterium | Mycobacterium |
| sp. 1100029.7 | sp. 1137317.9 | sp. 1164966.3 | sp. 1164985.4 | sp. 1165178.9 |
| Mycobacterium | Mycobacterium | Mycobacterium | Mycobacterium | Mycobacterium |
| sp. 1245111.1 | sp. 1245805.9 | sp. 1274756.6 | sp. 1274761.0 | sp. 141 |
| Mycobacterium | Mycobacterium | Mycobacterium | Mycobacterium | Mycobacterium |
| sp. 1423905.2 | sp. 1465703.0 | sp. 1477680.9 | sp. 1482268.1 | sp. 155 |
| Mycobacterium | Mycobacterium | Mycobacterium | Mycobacterium | Mycobacterium |
| sp. 1554424.7 | sp. 852002- | sp. 852002- | sp. 852002- | sp. 852002- |
| | 10029_SCH5224772 | 10318_SCH4845932 | 10379_SCH5584320 | 30065_SCH5024008 |
| Mycobacterium | Mycobacterium | Mycobacterium | Mycobacterium | Mycobacterium |
| sp. 852002- | sp. 852002- | sp. 852002- | sp. 852002- | sp. 852002- |
| 40037_SCH5390672 | 50816_SCH5313054-b | 51057_SCH5723018 | 51163_SCH5372311 | 51296_SCH5728562-a |
| Mycobacterium | Mycobacterium | Mycobacterium | Mycobacterium | Mycobacterium |
| sp. 852002- | sp. 852002- | sp. 852002- | sp. 852002- | sp. 852013- |
| 51613_SCH5001154 | 51759_SCH5129042 | 51971_SCH5477799-a | 53434_SCH5985345 | 50091_SCH5140682 |
| Mycobacterium | Mycobacterium | Mycobacterium | Mycobacterium | Mycobacterium |
| sp. 852013- | sp. 852013- | sp. 852014- | sp. 852014- | sp. 852014- |
| 50142_SCH4511227 | 51886_SCH5428379 | 50255_SCH5639931 | 51730_SCH5271717 | 52144_SCH5372336 |
| Mycobacterium | Mycobacterium | Mycobacterium | Mycobacterium | Mycobacterium |
| sp. 852014- | sp. AB215 | sp. AB308 | sp. AB57 | sp. ACS1612 |
| 52450_SCH5900713 | | | | |
| Mycobacterium | Mycobacterium | Mycobacterium | Mycobacterium | Mycobacterium |
| sp. ACS4054 | sp. ACS4331 | sp. ATI | sp. CECT 8766 | sp. D16Q16 |
| Mycobacterium | Mycobacterium | Mycobacterium | Mycobacterium | Mycobacterium |
| sp. D16R12 | sp. D16R18 | sp. djl-10 | sp. E136 | sp. E1386 |
| Mycobacterium | Mycobacterium | Mycobacterium | Mycobacterium | Mycobacterium |
| sp. E1747 | sp. E2238 | sp. E2327 | sp. E2462 | sp. E2479 |
| Mycobacterium | Mycobacterium | Mycobacterium | Mycobacterium | Mycobacterium |
| sp. E2497 | sp. E2699 | sp. E2733 | sp. E2989 | sp. E3198 |
| Mycobacterium | Mycobacterium | Mycobacterium | Mycobacterium | Mycobacterium |
| sp. E3247 | sp. E3298 | sp. E3339 | sp. E342 | sp. E740 |
| Mycobacterium | Mycobacterium | Mycobacterium | Mycobacterium | Mycobacterium |
| sp. E796 | sp. E802 | sp. EPa45 | sp. GA-1199 | sp. GA-1285 |
| Mycobacterium | Mycobacterium | Mycobacterium | Mycobacterium | Mycobacterium |
| sp. GA-1331 | sp. GA-1683 | sp. GA-1841 | sp. GA-1999 | sp. GA-2613 |
| Mycobacterium | Mycobacterium | Mycobacterium | Mycobacterium | Mycobacterium |
| sp. GA-2617 | sp. GA-2829 | sp. IEC1808 | sp. IS- | sp. IS- |
| | | | 1264 | 1496 |
| Mycobacterium | Mycobacterium | Mycobacterium | Mycobacterium | Mycobacterium |
| sp. IS-1590 | sp. IS-1742 | sp. IS-1744 | sp. IS-288 | sp. IS-3022 |
| Mycobacterium | Mycobacterium | Mycobacterium | Mycobacterium | Mycobacterium |
| sp. IS-836 | sp. JS623 | sp. M26 | sp. MS1601 | sp. NAZ1 90054 |
| Mycobacterium | Mycobacterium | Mycobacterium | Mycobacterium | Mycobacterium |
| sp. NS-7484 | sp. Root135 | sp. Root265 | sp. SCH | sp. Soil538 |
| Mycobacterium | Mycobacterium | Mycobacterium | Mycobacterium | Mycobacterium |
| sp. SP-6446 | sp. ST-F2 | sp. SWH-M1 | sp. SWH-M3 | sp. SWH-M5 |
| Mycobacterium | Mycobacterium | Mycobacterium | Mycobacterium | Mycobacterium |
| sp. TBL 1200985 | sp. UM_CSW | sp. UM_Kg1 | sp. UM_Kg17 | sp. UM_Kg27 |
| Mycobacterium | Mycobacterium | Mycobacterium | Mycobacterium | Mycobacterium |
| sp. UM_NZ2 | sp. UM_WGJ | sp. UM_WWY | sp. UNC267MFSha1.1M11 | sp. UNC280MFTsu5.1 |
| Mycobacterium | Mycobacterium | Mycobacterium | Mycobacterium | Mycobacterium |
| sp. URHB0044 | sp. URHD0025 | sp. VKM Ac-1816D | sp. VKM Ac-1817D | sp. WY10 |
| Mycobacterium | Mycobacterium | Mycobacterium | Mycobacterium | Mycobacterium |
| sp. YC-RL4 | sphagni | szulgai | talmoniae | terrae |
| Mycobacterium | Mycobacterium | Mycobacterium | Mycobacterium | Mycobacterium |
| thermoresistibile | timonense | triplex | triviale | tuberculosis |
| Mycobacterium | Mycobacterium | Mycobacterium | Mycobacterium | Mycobacterium |
| tusciae | vaccae | vanbaalenii | vulneris | wolinskyi |
| Mycobacterium | Mycoplasma | Mycoplasma | Mycoplasma | Mycoplasma |
| xenopi | agalactiae | agassizii | alkalescens | alligatoris |
| Mycoplasma | Mycoplasma | Mycoplasma | Mycoplasma | Mycoplasma |
| alvi | anatis | anseris | arginini | arthritidis |
| Mycoplasma | Mycoplasma | Mycoplasma | Mycoplasma | Mycoplasma |
| auris | bovigenitalium | bovis | bovoculi | buteonis |
| Mycoplasma | Mycoplasma | Mycoplasma | Mycoplasma | Mycoplasma |
| californicum | canadense | canis | capricolum | cloacale |
| Mycoplasma | Mycoplasma | Mycoplasma | Mycoplasma | Mycoplasma |
| collis | columbinum | columborale | conjunctivae | cricetuli |
| Mycoplasma | Mycoplasma | Mycoplasma | Mycoplasma | Mycoplasma |
| crocodyli | cynos | dispar | elephantis | felifaucium |
| Mycoplasma | Mycoplasma | Mycoplasma | Mycoplasma | Mycoplasma |
| felis | feriruminatoris | fermentans | flocculare | gallinarum |
| Mycoplasma | Mycoplasma | Mycoplasma | Mycoplasma | Mycoplasma |
| gallisepticum | glycophilum | haemocanis | hominis | hyopneumoniae |
| Mycoplasma | Mycoplasma | Mycoplasma | Mycoplasma | Mycoplasma |
| hyorhinis | hyosynoviae | imitans | iners | iowae |
| Mycoplasma | Mycoplasma | Mycoplasma | Mycoplasma | Mycoplasma |
| leachii | leonicaptivi | lipofaciens | meleagridis | mobile |
| Mycoplasma | Mycoplasma | Mycoplasma | Mycoplasma | Mycoplasma |
| molare | mycoides | opalescens | orale | ovipneumoniae |
| Mycoplasma | Mycoplasma | Mycoplasma | Mycoplasma | Mycoplasma |
| ovis | parvum | penetrans | pirum | pneumoniae |
| Mycoplasma | Mycoplasma | Mycoplasma | Mycoplasma | Mycoplasma |
| primatum | pullorum | pulmonis | putrefaciens | salivarium |
| Mycoplasma | Mycoplasma | Mycoplasma | Mycoplasma | Mycoplasma |
| simbae | sp. (ex | sp. HU2014 | spumans | sturni |
| | Biomphalaria | | | |
| | glabrata) | | | |
| Mycoplasma | Mycoplasma | Mycoplasma | Mycoplasma | Mycoplasma |
| synoviae | testudineum | testudinis | verecundum | yeatsii |
| Myroides | Myroides | Myroides | Myroides | Myroides |
| guanonis | injenensis | marinus | odoratimimus | odoratus |
| Myroides | Myxococcus | Myxococcus | Myxococcus | Myxococcus |
| phaeus | fulvus | hansupus | stipitatus | virescens |
| Myxococcus | Myxosarcina | Naegleria | Nafulsella | Nakamurella |
| xanthus | sp. G11 | gruberi | turpanensis | lactea |
| Nakamurella | Nakamurella | Nannizzia | Nannochloropsis | Natranaerobius |
| multipartita | panacisegetis | gypsea | gaditana | thermophilus |
| Natranaerobius | Natrialba | Natrialba | Natrialba | Natrialba |
| trueperi | aegyptia | asiatica | chahannaoensis | hulunbeirensis |
| Natrialba | Natrialba | Natrialba | Natrialbaceae | Natribacillus |
| magadii | sp. SSL1 | taiwanensis | archaeon JW/NM-HA | halophilus |
| | | | 15 | |
| Natrinema | Natrinema | Natrinema | Natrinema | Natrinema |
| altunense | pallidum | pellirubrum | salaciae | versiforme |
| Natronincola | Natronincola | Natronobacterium | Natronobacterium | Natronococcus |
| ferrireducens | peptidivorans | gregoryi | texcoconense | amylolyticus |
| Natronococcus | Natronococcus | Natronohydrobacter | Natronolimnobius | Natronolimnobius |
| jeotgali | occultus | thiooxidans | baerhuensis | innermongolicus |
| Natronomonas | Natronomonas | Natronomonas | Natronorubrum | Natronorubrum |
| moolapensis | pharaonis | sp. CBA1134 | bangense | sulfidifaciens |
| Natronorubrum | Natronorubrum | Natronorubrum | Naumovozyma | Naumovozyma |
| texcoconense | thiooxidans | tibetense | castellii | dairenensis |
| Nautella | Nautella | Nautilia | Ndongobacter | Necropsobacter |
| italica | sp. ECSMB14104 | profundicola | massiliensis | massiliensis |
| Necropsobacter | Nectria | Negativibacillus | Negativicoccus | Neglecta |
| rosorum | haematococca | massiliensis | massiliensis | timonensis |
| Neiella | Neisseria | Neisseria | Neisseria | Neisseria |
| marina | animaloris | arctica | bacilliformis | canis |
| Neisseria | Neisseria | Neisseria | Neisseria | Neisseria |
| cinerea | dentiae | elongata | flavescens | gonorrhoeae |
| Neisseria | Neisseria | Neisseria | Neisseria | Neisseria |
| lactamica | macacae | meningitidis | mucosa | polysaccharea |
| Neisseria | Neisseria | Neisseria | Neisseria | Neisseria |
| shayeganii | sicca | sp. 10023 | sp. 114725 | sp. 124861 |
| Neisseria | Neisseria | Neisseria | Neisseria | Neisseria |
| sp. 74A18 | sp. 83E34 | sp. 93087 | sp. HMSC03D10 | sp. HMSC055H02 |
| Neisseria | Neisseria | Neisseria | Neisseria | Neisseria |
| sp. HMSC056A03 | sp. HMSC058F07 | sp. HMSC059F02 | sp. HMSC061B04 | sp. HMSC061E12 |
| Neisseria | Neisseria | Neisseria | Neisseria | Neisseria |
| sp. HMSC061H08 | sp. HMSC063B05 | sp. HMSC064D07 | sp. HMSC064E01 | sp. HMSC064F03 |
| Neisseria | Neisseria | Neisseria | Neisseria | Neisseria |
| sp. HMSC064F04 | sp. HMSC065C04 | sp. HMSC065D04 | sp. HMSC066B07 | sp. HMSC066H01 |
| Neisseria | Neisseria | Neisseria | Neisseria | Neisseria |
| sp. HMSC068C04 | sp. HMSC068C12 | sp. HMSC069H12 | sp. HMSC070A01 | sp. HMSC070E12 |
| Neisseria | Neisseria | Neisseria | Neisseria | Neisseria |
| sp. HMSC070H10 | sp. HMSC071C03 | sp. HMSC072B12 | sp. HMSC072C05 | sp. HMSC072F04 |
| Neisseria | Neisseria | Neisseria | Neisseria | Neisseria |
| sp. HMSC073B07 | sp. HMSC073G10 | sp. HMSC074B07 | sp. HMSC075C10 | sp. HMSC077D05 |
| Neisseria | Neisseria | Neisseria | Neisseria | Neisseria |
| sp. HMSC078C12 | sp. HMSC15C08 | sp. HMSC15G01 | sp. HMSC31F04 | sp. HMSC70E02 |
| Neisseria | Neisseria | Neisseria | Neisseria | Neisseria |
| sp. N95_16 | sp. oral | sp. oral | subflava | wadsworthii |
| | taxon 014 | taxon 020 | | |
| Neisseria | Neisseria | Nematocida | Neoasaia | Neochlamydia |
| weaveri | zoodegmatis | parisii | chiangmaiensis | sp. S13 |
| Neochlamydia | Neofamilia | Neofusicoccum | Neokomagataea | Neomicrococcus |
| sp. TUME1 | massiliensis | parvum | thailandica | aestuarii |
| Neorhizobium | Neorickettsia | Neorickettsia | Neorickettsia | Neospora |
| galegae | helminthoeca | sennetsu | sp. 179522 | caninum |
| Neosynechococcus | Neptuniibacter | Neptuniibacter | Neptuniibacter | Neptunomonas |
| sphagnicola | caesariensis | marinus | pectenicola | antarctica |
| Neptunomonas | Neptunomonas | Neptunomonas | Nereida | Nesiotobacter |
| japonica | phycophila | qingdaonensis | ignava | exalbescens |
| Nesterenkonia | Nesterenkonia | Nesterenkonia | Nesterenkonia | Nesterenkonia |
| alba | jeotgali | massiliensis | sp. AN1 | sp. F |
| Nesterenkonia | Neurospora | Neurospora | Nevskia | Nevskia |
| sp. PF2B19 | crassa | tetrasperma | ramosa | soli |
| Niabella | Niabella | Niabella | Niabella | Niameybacter |
| aurantiaca | drilacis | ginsenosidivorans | soli | massiliensis |
| Niastella | Niastella | Niastella | Niastella | Nioella |
| koreensis | populi | vici | yeongjuensis | nitratireducens |
| Nioella | Nisaea | Nissabacter | Nitratifractor | Nitratireductor |
| sediminis | denitrificans | archeti | salsuginis | aquibiodomus |
| Nitratireductor | Nitratireductor | Nitratireductor | Nitratireductor | Nitratireductor |
| basaltis | indicus | pacificus | soli | sp. ES.061 |
| Nitratiruptor | Nitratiruptor | Nitriliruptor | Nitrincola | Nitrincola |
| sp. SB155-2 | tergarcus | alkaliphilus | lacisaponensis | nitratireducens |
| Nitrincola | Nitritalea | Nitrobacter | Nitrobacter | Nitrobacter |
| sp. A-D6 | halalkaliphila | hamburgensis | sp. Nb-311A | vulgaris |
| Nitrobacter | Nitrococcus | Nitrolancea | Nitrosococcus | Nitrosococcus |
| winogradskyi | mobilis | hollandica | halophilus | oceani |
| Nitrosococcus | Nitrosomonas | Nitrosomonas | Nitrosomonas | Nitrosomonas |
| watsonii | aestuarii | communis | cryotolerans | europaea |
| Nitrosomonas | Nitrosomonas | Nitrosomonas | Nitrosomonas | Nitrosomonas |
| eutropha | halophila | marina | mobilis | nitrosa |
| Nitrosomonas | Nitrosomonas | Nitrosomonas | Nitrosomonas | Nitrosomonas |
| oligotropha | sp. AL212 | sp. Is79A3 | sp. Nm132 | sp. Nm166 |
| Nitrosomonas | Nitrosomonas | Nitrosomonas | Nitrosomonas | Nitrosomonas |
| sp. Nm33 | sp. Nm34 | sp. Nm51 | sp. Nm58 | ureae |
| Nitrosopiimilus | Nitrososphaera | Nitrosospira | Nitrosospira | Nitrosospira |
| sp. Nsub | viennensis | briensis | lacus | multiformis |
| Nitrosospira | Nitrosospira | Nitrosospira | Nitrosospira | Nitrosospira |
| sp. Nl5 | sp. NpAV | sp. Nsp1 | sp. Nsp11 | sp. Nsp13 |
| Nitrosospira | Nitrosospira | Nitrosovibrio | Nitrosovibrio | Nitrosovibrio |
| sp. Nsp14 | sp. Nsp18 | sp. Nv17 | sp. Nv6 | tenuis |
| Nitrospina | Nitrospira | Nitrospira | Nitrospira | Nitrospira |
| gracilis | cf. moscoviensis SBR1015 | defluvii | japonica | moscoviensis |
| Nitrospira | Nitrospirae | Nitrospirillum | Niveispirillum | Niveispirillum |
| sp. ND1 | bacterium | amazonense | irakense | sp. TH1-14 |
| | HCH-1 | | | |
| Nocardia | Nocardia | Nocardia | Nocardia | Nocardia |
| abscessus | acidivorans | africana | alba | altamirensis |
| Nocardia | Nocardia | Nocardia | Nocardia | Nocardia |
| amamiensis | amikacinitolerans | anaemiae | aobensis | araoensis |
| Nocardia | Nocardia | Nocardia | Nocardia | Nocardia |
| arizonensis | arthritidis | asiatica | beijingensis | brasiliensis |
| Nocardia | Nocardia | Nocardia | Nocardia | Nocardia |
| brevicatena | caishijiensis | carnea | cerradoensis | concava |
| Nocardia | Nocardia | Nocardia | Nocardia | Nocardia |
| coubleae | crassostreae | cummidelens | cyriacigeorgica | donostiensis |
| Nocardia | Nocardia | Nocardia | Nocardia | Nocardia |
| elegans | exalbida | farcinica | flavorosea | fusca |
| Nocardia | Nocardia | Nocardia | Nocardia | Nocardia |
| gamkensis | grenadensis | harenae | higoensis | ignorata |
| Nocardia | Nocardia | Nocardia | Nocardia | Nocardia |
| inohanensis | jejuensis | jiangxiensis | jinanensis | kruczakiae |
| Nocardia | Nocardia | Nocardia | Nocardia | Nocardia |
| lijiangensis | mexicana | mikamii | miyunensis | niigatensis |
| Nocardia | Nocardia | Nocardia | Nocardia | Nocardia |
| niwae | nova | otitidiscaviarum | paucivorans | pneumoniae |
| Nocardia | Nocardia | Nocardia | Nocardia | Nocardia |
| pseudobrasiliensis | pseudovaccinii | puris | rhamnosiphila | salmonicida |
| Nocardia | Nocardia | Nocardia | Nocardia | Nocardia |
| seriolae | shimofusensis | sienata | soli | sp. 348MFTsu5.1 |
| Nocardia | Nocardia | Nocardia | Nocardia | Nocardia |
| sp. 852002- | sp. 852002- | sp. 852002- | sp. BMG111209 | sp. BMG51109 |
| 20019_SCH5090214 | 51101_SCH5132738 | 51244_SCH5132740 | | |
| Nocardia | Nocardia | Nocardia | Nocardia | Nocardia |
| sp. CNY236 | sp. NRRL S- | sp. NRRL | sp. Root136 | speluncae |
| | 836 | WC-3656 | | |
| Nocardia | Nocardia | Nocardia | Nocardia | Nocardia |
| takedensis | tenerifensis | terpenica | testacea | thailandica |
| Nocardia | Nocardia | Nocardia | Nocardia | Nocardia |
| transvalensis | uniformis | vaccinii | vermiculata | veterana |
| Nocardia | Nocardia | Nocardia | Nocardia | Nocardioides |
| vinacea | vulneris | xishanensis | yamanashiensis | alkalitolerans |
| Nocardioides | Nocardioides | Nocardioides | Nocardioides | Nocardioides |
| alpinus | dokdonensis | exalbidus | halotolerans | insulae |
| Nocardioides | Nocardioides | Nocardioides | Nocardioides | Nocardioides |
| jensenii | lianchengensis | luteus | psychrotolerans | sp. CF8 |
| Nocardioides | Nocardioides | Nocardioides | Nocardioides | Nocardioides |
| sp. Iso805N | sp. J54 | sp. Leaf285 | sp. Leaf307 | sp. Root122 |
| Nocardioides | Nocardioides | Nocardioides | Nocardioides | Nocardioides |
| sp. Root140 | sp. Root151 | sp. Root190 | sp. Soil774 | sp. Soil777 |
| Nocardioides | Nocardioides | Nocardioides | Nocardioides | Nocardioides |
| sp. Soil796 | sp. Soil797 | sp. Soil805 | sp. URHA0020 | sp. URHA0032 |
| Nocardioides | Nocardioides | Nocardioides | Nocardiopsis | Nocardiopsis |
| sp. YR527 | szechwanensis | terrae | alba | alkaliphila |
| Nocardiopsis | Nocardiopsis | Nocardiopsis | Nocardiopsis | Nocardiopsis |
| baichengensis | chromatogenes | dassonvillei | flavescens | ganjiahuensis |
| Nocardiopsis | Nocardiopsis | Nocardiopsis | Nocardiopsis | Nocardiopsis |
| gilva | halophila | halotolerans | kunsanensis | listeri |
| Nocardiopsis | Nocardiopsis | Nocardiopsis | Nocardiopsis | Nocardiopsis |
| lucentensis | potens | prasina | salina | sinuspersici |
| Nocardiopsis | Nocardiopsis | Nocardiopsis | Nocardiopsis | Nocardiopsis |
| sp. CNR- | sp. CNS- | sp. CNT312 | sp. JB363 | sp. NRRL |
| 923 | 639 | | | B-16309 |
| Nocardiopsis | Nocardiopsis | Nocardiopsis | Nocardiopsis | Nocardiopsis |
| sp. RV163 | sp. SBT366 | sp. TP- | sp. TSRI0078 | synnemataformans |
| | | A0876 | | |
| Nocardiopsis | Nocardiopsis | Nocardiopsis | Nodosilinea | Nodularia |
| trehalosi | valliformis | xinjiangensis | nodulosa | sp. NIES- |
| | | | | 3585 |
| Nodularia | Nonlabens | Nonlabens | Nonlabens | Nonlabens |
| spumigena | dokdonensis | marinus | sp. Hell_33_55 | sp. M1C269 |
| Nonlabens | Nonlabens | Nonlabens | Nonomuraea | Nonomuraea |
| sp. YIK11 | spongiae | ulvanivorans | Candida | coxensis |
| Nonomuraea | Nonomuraea | Nonomuraea | Nonomuraea | Nonomuraea |
| jiangxiensis | maritima | pusilla | sp. ATCC | sp. NBRC |
| | | | 55076 | 110462 |
| Nonomuraea | Nonomuraea | Nosema | Nosocomiicoccus | Nosocomiicoccus |
| sp. SBT364 | wenchangensis | ceranae | ampullae | massiliensis |
| Nosocomiicoccus | Nostoc | Nostoc | Nostoc | Nostoc |
| sp. HMSC09A07 | calcicola | piscinale | punctiforme | sp. 106C |
| Nostoc | Nostoc | Nostoc | Nostoc | Nostoc |
| sp. KVJ20 | sp. NIES-3756 | sp. PCC 7107 | sp. PCC 7120 | sp. PCC 7524 |
| Nostoc | Nostoc | Nostoc | Nostoc | Nostoc |
| sp. ‘Peltigera | sp. ‘Peltigera | sp. ‘Peltigera | sp. RF31Y | sp. T09 |
| membranacea | membranacea | membranacea | | |
| cyanobiont’ 210A | cyanobiont’ 213 | cyanobiont’ 232 | | |
| Nostocales | Notoacmeibacter | Novibacillus | Noviherbaspirillum | Noviherbaspirillum |
| cyanobacterium HT- | marinus | thermophilus | autotrophicum | denitrificans |
| 58-2 | | | | |
| Noviherbaspirillum | Noviherbaspirillum | Noviherbaspirillum | Novispirillum | Novosphingobium |
| humi | massiliense | sp. Root189 | itersonii | acidiphilum |
| Novosphingobium | Novosphingobium | Novosphingobium | Novosphingobium | Novosphingobium |
| barchaimii | capsulatum | fuchskuhlense | lentum | lindaniclasticum |
| Novosphingobium | Novosphingobium | Novosphingobium | Novosphingobium | Novosphingobium |
| malaysiense | mathurense | naphthalenivorans | nitrogenifigens | panipatense |
| Novosphingobium | Novosphingobium | Novosphingobium | Novosphingobium | Novosphingobium |
| pentaromativorans | resinovorum | rosa | sp. AAP1 | sp. AAP83 |
| Novosphingobium | Novosphingobium | Novosphingobium | Novosphingobium | Novosphingobium |
| sp. AAP93 | sp. AP12 | sp. B 225 | sp. B1 | sp. B-7 |
| Novosphingobium | Novosphingobium | Novosphingobium | Novosphingobium | Novosphingobium |
| sp. CCH12-A3 | sp. CF614 | sp. FSW06-99 | sp. Fuku2-lSO-50 | sp. KN65.2 |
| Novosphingobium | Novosphingobium | Novosphingobium | Novosphingobium | Novosphingobium |
| sp. Leaf2 | sp. MBES04 | sp. NDB2Meth1 | sp. P6W | sp. PP1Y |
| Novosphingobium | Novosphingobium | Novosphingobium | Novosphingobium | Niimidum |
| sp. Rr 2-17 | sp. ST904 | subterraneum | tardaugens | massiliense |
| Obesumbacterium | Oblitimonas | Occidentia | Oceanibacterium | Oceanibaculum |
| proteus | alkaliphila | massiliensis | hippocampi | indicum |
| Oceanibaculum | Oceanibaculum | Oceanibulbus | Oceanicaulis | Oceanicaulis |
| pacificum | sp. L54-1-50 | indolifex | alexandrii | sp. HL-87 |
| Oceanicaulis | Oceanicoccus | Oceanicola | Oceanicola | Oceanicola |
| sp. HTCC2633 | sagamiensis | granulosus | litoreus | sp. 22II- |
| | | | | s10i |
| Oceanicola | Oceanicola | Oceanicola | Oceanimonas | Oceanimonas |
| sp. HL-35 | sp. MCTG156(1a) | sp. S124 | baumannii | doudoroffii |
| Oceanimonas | Oceanimonas | Oceaniovalibus | Oceanisphaera | Oceanisphaera |
| smirnovii | sp. GK1 | guishaninsula | profunda | psychrotolerans |
| Oceanisphaera | Oceanithermus | Oceanivirga | Oceanobacillus | Oceanobacillus |
| sp. AMac2203 | profundus | salmonicida | caeni | damuensis |
| Oceanobacillus | Oceanobacillus | Oceanobacillus | Oceanobacillus | Oceanobacillus |
| iheyensis | jeddahense | kimchii | limi | manasiensis |
| Oceanobacillus | Oceanobacillus | Oceanobacillus | Oceanobacillus | Oceanobacillus |
| massiliensis | oncorhynchi | picturae | rekensis | senegalensis |
| Oceanobacillus | Oceanobacillus | Oceanobacillus | Oceanobacillus | Oceanobacter |
| sojae | sp. Castelsardo | sp. E9 | timonensis | kriegii |
| Oceanococcus | Oceanospirillum | Oceanospirillum | Oceanospirillum | Oceanospirillum |
| atlanticus | beijerinckii | linum | maris | multiglobuliferum |
| Oceanospirillum | Ochrobactrum | Ochrobactrum | Ochrobactrum | Ochrobactrum |
| sanctuarii | anthropi | cytisi | grignonense | intermedium |
| Ochrobactrum | Ochrobactrum | Ochrobactrum | Ochrobactrum | Ochrobactrum |
| lupini | pituitosum | pseudogrignonense | rhizosphaerae | sp. A44 |
| Ochrobactrum | Ochrobactrum | Ochrobactrum | Ochrovirga | Octadecabacter |
| sp. EGD-AQ16 | sp. P6BS-III | thiophenivorans | pacifica | antarcticus |
| Octadecabacter | Octadecabacter | Octadecabacter | Odoribacter | Odoribacter |
| arcticus | ascidiaceicola | temperatus | laneus | splanchnicus |
| Oenococcus | Oenococcus | Oerskovia | Oerskovia | Oerskovia |
| kitaharae | oeni | enterophila | sp. Root22 | sp. Root918 |
| Oerskovia | Ogataea | Ogataea | Ohtaekwangia | Okibacterium |
| turbata | parapolymorpha | polymorpha | koreensis | fritillariae |
| Olegusella | Oleiagrimonas | Oleibacter | Oleiphilus | Oleiphilus |
| massiliensis | soli | marinas | messinensis | sp. HI0080 |
| Oleiphilus | Oleispira | Oligella | Oligella | Oligella |
| sp. HI0123 | antarctica | sp. HMSC05A10 | ureolytica | urethralis |
| Oligotropha | Olivibacter | Olleya | Olleya | Olleya |
| carboxidovorans | sitiensis | marilimosa | namhaensis | sp. 1TB9 |
| Olleya | Olsenella | Olsenella | Olsenella | Olsenella |
| sp. VCSA23 | profusa | scatoligenes | sp. An188 | sp. An270 |
| Olsenella | Olsenella | Olsenella | Olsenella | Olsenella |
| sp. An285 | sp. An290 | sp. An293 | sp. DNF00959 | sp. HMSC062G07 |
| Olsenella | Olsenella | Olsenella | Olsenella | Olsenella |
| sp. kh2p3 | sp. KH3B4 | sp. Marseille-P2300 | sp. Marseille-P2912 | sp. Marseille-P2936 |
| Olsenella | Olsenella | Olsenella | Olsenella | Olsenella |
| sp. Marseille-P3197 | sp. Marseille-P3256 | sp. oral | sp. oral | sp. SIT9 |
| | | taxon 807 | taxon 809 | |
| Olsenella | Olsenella | Opitutaceae bacterium | Opitutaceae bacterium | Opitutaceae bacterium |
| uli | umbonata | TAV1 | TAV3 | TAV4 |
| Opitutaceae bacterium | Opitutaceae bacterium | Opitutus | Opitutus | Ordospora |
| TAV5 | TSB47 | sp. GAS368 | terrae | colligata |
| Orenia | Orenia | Oribacterium | Oribacterium | Oribacterium |
| marismortui | metallireducens | asaccharolyticum | parvum | sinus |
| Oribacterium | Oribacterium | Oribacterium | Oribacterium | Oribacterium |
| sp. C9 | sp. FC2011 | sp. KHPX15 | sp. NK2B42 | sp. oral |
| | | | | taxon 078 |
| Oribacterium | Oribacterium | Oribacterium | Orientia | Orientia |
| sp. oral | sp. P6A1 | sp. WCC10 | chuto | tsutsugamushi |
| taxon 108 | | | | |
| Ornatilinea | Ornithinibacillus | Ornithinibacillus | Ornithinibacillus | Ornithinibacillus |
| apprima | californiensis | contaminans | halophilus | scapharcae |
| Omithinimicrobium | Omithinimicrobium | Ornithobacterium | Orrella | Oscillatoria |
| pekingense | sp. CNJ-824 | rhinotracheale | dioscoreae | acuminata |
| Oscillatoria nigro- | Oscillatoria | Oscillatoriales | Oscillatoriales | Oscillibacter |
| viridis | sp. PCC | cyanobacterium JSC- | cyanobacterium MTP1 | ruminantium |
| | 10802 | 12 | | |
| Oscillibacter | Oscillibacter | Oscillibacter | Oscillibacter | Oscillibacter |
| sp. 1-3 | sp. ER4 | sp. KLE | sp. KLE | sp. PC13 |
| | | 1728 | 1745 | |
| Oscillibacter | Oscillochloris | Oscillospiraceae | Ostreid | Ottowia |
| valericigenes | trichoides | bacterium VE202-24 | herpesvirus 1 | sp. oral taxon |
| | | | | 894 |
| Ottowia | Owenweeksia | Oxalobacter | Oxalobacteraceae | Oxalobacteraceae |
| thiooxydans | hongkongensis | formigenes | bacterium AB‘14 | bacterium IMCC9480 |
| Oxobacter | Pacificibacter | Pacificimonas | Paenalcaligenes | Paenarthrobacter |
| pfennigii | marinus | flava | hominis | aurescens |
| Paenarthrobacter | Paenibacillaceae | Paenibacillus | Paenibacillus | Paenibacillus |
| nicotinovorans | bacterium GAS479 | alginolyticus | algorifonticola | alvei |
| Paenibacillus | Paenibacillus | Paenibacillus | Paenibacillus | Paenibacillus |
| amylolyticus | antarcticus | antibioticophila | apiarius | assamensis |
| Paenibacillus | Paenibacillus | Paenibacillus | Paenibacillus | Paenibacillus |
| barengoltzii | beijingensis | borealis | bovis | camerounensis |
| Paenibacillus | Paenibacillus | Paenibacillus | Paenibacillus | Paenibacillus |
| campinasensis | catalpae | chitinolyticus | chondroitinus | curdlanolyticus |
| Paenibacillus | Paenibacillus | Paenibacillus | Paenibacillus | Paenibacillus |
| daejeonensis | darwinianus | dauci | dendritiformis | donghaensis |
| Paenibacillus | Paenibacillus | Paenibacillus | Paenibacillus | Paenibacillus |
| durus | ehimensis | elgii | etheri | ferrarius |
| Paenibacillus | Paenibacillus | Paenibacillus | Paenibacillus | Paenibacillus |
| fonticola | forsythiae | ginsengihumi | glacialis | glucanolyticus |
| Paenibacillus | Paenibacillus | Paenibacillus | Paenibacillus | Paenibacillus |
| gorillae | graminis | harenae | herberti | ihbetae |
| Paenibacillus | Paenibacillus | Paenibacillus | Paenibacillus | Paenibacillus |
| ihumii | jamilae | jilunlii | kribbensis | lactis |
| Paenibacillus | Paenibacillus | Paenibacillus | Paenibacillus | Paenibacillus |
| larvae | lautus | macerans | macquariensis | massiliensis |
| Paenibacillus | Paenibacillus | Paenibacillus | Paenibacillus | Paenibacillus |
| mucilaginosus | naphthalenovorans | odorifer | oryzae | pabuli |
| Paenibacillus | Paenibacillus | Paenibacillus | Paenibacillus | Paenibacillus |
| panacisoli | pasadenensis | pectinilyticus | peoriae | phocaensis |
| Paenibacillus | Paenibacillus | Paenibacillus | Paenibacillus | Paenibacillus |
| physcomitrellae | pini | pinihumi | polymyxa | polysaccharolyticus |
| Paenibacillus | Paenibacillus | Paenibacillus | Paenibacillus | Paenibacillus |
| popilliae | rhizosphaerae | rigui | riograndensis | rubinfantis |
| Paenibacillus | Paenibacillus | Paenibacillus | Paenibacillus | Paenibacillus |
| sabinae | sanguinis | selenitireducens | senegalensis | senegalimassiliensis |
| Paenibacillus | Paenibacillus | Paenibacillus | Paenibacillus | Paenibacillus |
| sonchi | sophorae | sp. 1‘12 | sp. 11 | sp. 1-18 |
| Paenibacillus | Paenibacillus | Paenibacillus | Paenibacillus | Paenibacillus |
| sp. 1-49 | sp. 181mfcol5.1 | sp. 1ZS3-15 | sp. 276b | sp. 32352 |
| Paenibacillus | Paenibacillus | Paenibacillus | Paenibacillus | Paenibacillus |
| sp. 32O-W | sp. 453mf | sp. 7516 | sp. 7523-1 | sp. 7541 |
| Paenibacillus | Paenibacillus | Paenibacillus | Paenibacillus | Paenibacillus |
| sp. 7884-2 | sp. A3 | sp. A59 | sp. A9 | sp. AD87 |
| Paenibacillus | Paenibacillus | Paenibacillus | Paenibacillus | Paenibacillus |
| sp. Aloe-11 | sp. BC26 | sp. CF095 | sp. CF384 | sp. c1141a |
| Paenibacillus | Paenibacillus | Paenibacillus | Paenibacillus | Paenibacillus |
| sp. cl6col | sp. D9 | sp. DMB20 | sp. DMB5 | sp. E194 |
| Paenibacillus | Paenibacillus | Paenibacillus | Paenibacillus | Paenibacillus |
| sp. FF9 | sp. FJAT-22460 | sp. FJAT-26967 | sp. FJAT-27812 | sp. FSL |
| | | | | A5-0031 |
| Paenibacillus | Paenibacillus | Paenibacillus | Paenibacillus | Paenibacillus |
| sp. FSL | sp. FSL | sp. FSL | sp. FSL | sp. FSL |
| H7-0326 | H7-0331 | H7-0357 | H7-0737 | H7-689 |
| Paenibacillus | Paenibacillus | Paenibacillus | Paenibacillus | Paenibacillus |
| sp. FSL | sp. FSL | sp. FSL | sp. FSL | sp. FSL |
| H8-0259 | H8-0548 | H8-457 | P4-0081 | R5-0345 |
| Paenibacillus | Paenibacillus | Paenibacillus | Paenibacillus | Paenibacillus |
| sp. FSL | sp. FSL | sp. FSL | sp. FSL | sp. FSL |
| R5-0490 | R5-0765 | R5-0912 | R5-192 | R7-0273 |
| Paenibacillus | Paenibacillus | Paenibacillus | Paenibacillus | Paenibacillus |
| sp. FSL | sp. FSL | sp. FSL | sp. FSL | sp. FSL |
| R7-0331 | R7-0333 | R7-0337 | R7-269 | R7-277 |
| Paenibacillus | Paenibacillus | Paenibacillus | Paenibacillus | Paenibacillus |
| sp. GD6 | sp. GM2 | sp. GP183 | sp. HGF5 | sp. HW567 |
| Paenibacillus | Paenibacillus | Paenibacillus | Paenibacillus | Paenibacillus |
| sp. IHB | sp. IHBB | sp. J14 | sp. J6 | sp. JCM |
| B 3415 | 10380 | | | 10914 |
| Paenibacillus | Paenibacillus | Paenibacillus | Paenibacillus | Paenibacillus |
| sp. JDR-2 | sp. KS1 | sp. LC231 | sp. Leaf72 | sp. LPB0068 |
| Paenibacillus | Paenibacillus | Paenibacillus | Paenibacillus | Paenibacillus |
| sp. Mc5Re-14 | sp. MY03 | sp. NAIST15-1 | sp. NFR01 | sp. O199 |
| Paenibacillus | Paenibacillus | Paenibacillus | Paenibacillus | Paenibacillus |
| sp. OK003 | sp. OK060 | sp. OK076 | sp. oral | sp. OSY-SE |
| | | | taxon 786 | |
| Paenibacillus | Paenibacillus | Paenibacillus | Paenibacillus | Paenibacillus |
| sp. ov031 | sp. OV219 | sp. P1XP2 | sp. P22 | sp. P26E |
| Paenibacillus | Paenibacillus | Paenibacillus | Paenibacillus | Paenibacillus |
| sp. P32E | sp. P3E | sp. P46E | sp. PAMC 26794 | sp. PDC88 |
| Paenibacillus | Paenibacillus | Paenibacillus | Paenibacillus | Paenibacillus |
| sp. Root444D2 | sp. Root52 | sp. RU5A | sp. RUD330 | sp. Soil522 |
| Paenibacillus | Paenibacillus | Paenibacillus | Paenibacillus | Paenibacillus |
| sp. Soil724D2 | sp. Soil750 | sp. Soil766 | sp. Soil787 | sp. SSG-1 |
| Paenibacillus | Paenibacillus | Paenibacillus | Paenibacillus | Paenibacillus |
| sp. St-s | sp. TCA20 | sp. TI45-13ar | sp. UNC217MF | sp. UNC451MF |
| Paenibacillus | Paenibacillus | Paenibacillus | Paenibacillus | Paenibacillus |
| sp. UNC496MF | sp. URHA0014 | sp. VKM | sp. VT- | sp. VT- |
| | | B-2647 | 16-81 | 400 |
| Paenibacillus | Paenibacillus | Paenibacillus | Paenibacillus | Paenibacillus |
| sp. VTT | sp. VTT | sp. XY044 | sp. Y412MC10 | sp. yr247 |
| E-133280 | E-133291 | | | |
| Paenibacillus | Paenibacillus | Paenibacillus | Paenibacillus | Paenibacillus |
| stellifer | swuensis | taichungensis | taiwanensis | terrae |
| Paenibacillus | Paenibacillus | Paenibacillus | Paenibacillus | Paenibacillus |
| terrigena | thiaminolyticus | tianmuensis | tuaregi | typhae |
| Paenibacillus | Paenibacillus | Paenibacillus | Paenibacillus | Paenibacillus |
| tyrfis | wulumuqiensis | wynnii | xylanexedens | yonginensis |
| Paenibacillus | Paeniclostridium | Paeniglutamicibacter | Paeniglutamicibacter | Paenirhodobacter |
| zanthoxyli | sordellii | antarcticus | gangotriensis | enshiensis |
| Paenirhodobacter | Paenisporosarcina | Paenisporosarcina | Paenisporosarcina | Paenisporosarcina |
| sp. MME-103 | indica | quisquiliarum | sp. HGH0030 | sp. TG-14 |
| Paenisporosarcina | Pajaroellobacter | Palaeococcus | Palleronia | Paludibacter |
| sp. TG20 | abortibovis | ferrophilus | marisminoris | jiangxiensis |
| Paludibacter | Paludibacterium | Paludifilum | Paludisphaera | Pandoraea |
| propionicigenes | yongneupense | halophilum | borealis | apista |
| Pandoraea | Pandoraea | Pandoraea | Pandoraea | Pandoraea |
| faecigallinarum | norimbergensis | oxalativorans | pnomenusa | pulmonicola |
| Pandoraea | Pandoraea | Pandoraea | Pandoraea | Pandoraea |
| sp. B-6 | sp. ISTKB | sp. PE- | sp. PE- | sp. SD6-2 |
| | | S2R-1 | S2T-3 | |
| Pandoraea | Pandoraea | Pandoraea | Pandoravirus | Pannonibacter |
| sputorum | thiooxydans | vervacti | dulcis | indicus |
| Pannonibacter | Pantoea | Pantoea | Pantoea | Pantoea |
| phragmitetus | agglomerans | alhagi | ananatis | anthophila |
| Pantoea | Pantoea | Pantoea | Pantoea | Pantoea |
| calida | conspicua | cypripedii | dispersa | eucrina |
| Pantoea | Pantoea | Pantoea | Pantoea | Pantoea |
| latae | rwandensis | septica | sp. 1.19 | sp. 3.5.1 |
| Pantoea | Pantoea | Pantoea | Pantoea | Pantoea |
| sp. 9140 | sp. A4 | sp. aB | sp. AS- | sp. At-9b |
| | | | PWVM4 | |
| Pantoea | Pantoea | Pantoea | Pantoea | Pantoea |
| sp. BL1 | sp. CFSAN033090 | sp. GM01 | sp. IMH | sp. NGS-ED- |
| | | | | 1003 |
| Pantoea | Pantoea | Pantoea | Pantoea | Pantoea |
| sp. OV426 | sp. OXWO6B1 | sp. PSNIH1 | sp. PSNIH2 | sp. RIT-PI-b |
| Pantoea | Pantoea | Pantoea | Pantoea | Pantoea |
| sp. Sc1 | sp. SL1_M5 | sp. SM3 | sp. VS1 | stewartii |
| Pantoea | Papillibacter | Parabacteroides | Parabacteroides | Parabacteroides |
| vagans | cinnamivorans | chartae | distasonis | goldsteinii |
| Parabacteroides | Parabacteroides | Parabacteroides | Parabacteroides | Parabacteroides |
| gordonii | johnsonii | merdae | phage YZ-2015b | sp. 2_1-7 |
| Parabacteroides | Parabacteroides | Parabacteroides | Parabacteroides | Parabacteroides |
| sp. 20_3 | sp. An277 | sp. ASF519 | sp. AT13 | sp. CT06 |
| Parabacteroides | Parabacteroides | Parabacteroides | Parabacteroides | Parabacteroides |
| sp. D13 | sp. D25 | sp. D26 | sp. HGS0025 | sp. Marseille-P3160 |
| Parabacteroides | Parabacteroides | Paraburkholderia | Paraburkholderia | Paraburkholderia |
| sp. Marseille-P3236 | sp. SN4 | acidipaludis | andropogonis | aspalathi |
| Paraburkholderia | Paraburkholderia | Paraburkholderia | Paraburkholderia | Paraburkholderia |
| bannensis | bryophila | caballeronis | caledonica | caribensis |
| Paraburkholderia | Paraburkholderia | Paraburkholderia | Paraburkholderia | Paraburkholderia |
| caryophylli | diazotrophica | dilworthii | ferrariae | fungorum |
| Paraburkholderia | Paraburkholderia | Paraburkholderia | Paraburkholderia | Paraburkholderia |
| ginsengisoli | ginsengiterrae | graminis | heleia | hospita |
| Paraburkholderia | Paraburkholderia | Paraburkholderia | Paraburkholderia | Paraburkholderia |
| kururiensis | megapolitana | mimosarum | monticola | nodosa |
| Paraburkholderia | Paraburkholderia | Paraburkholderia | Paraburkholderia | Paraburkholderia |
| oxyphila | panaciterrae | phenazinium | phenoliruptrix | phymatum |
| Paraburkholderia | Paraburkholderia | Paraburkholderia | Paraburkholderia | Paraburkholderia |
| phytofirmans | piptadeniae | rhizoxinica | ribeironis | sacchari |
| Paraburkholderia | Paraburkholderia | Paraburkholderia | Paraburkholderia | Paraburkholderia |
| sartisoli | sp. BN5 | sp. SOS3 | sprentiae | susongensis |
| Paraburkholderia | Paraburkholderia | Paraburkholderia | Paraburkholderia | Parachlamydia |
| terrae | tropica | tuberum | xenovorans | sp. C2 |
| Parachlamydiaceae | Paraclostridium | Paraclostridium | Paracoccidioides | Paracoccidioides |
| bacterium HS-T3 | benzoelyticum | bifermentans | brasiliensis | lutzii |
| Paracoccus | Paracoccus | Paracoccus | Paracoccus | Paracoccus |
| alcaliphilus | alkenifer | aminophilus | aminovorans | chinensis |
| Paracoccus | Paracoccus | Paracoccus | Paracoccus | Paracoccus |
| contaminans | denitrificans | halophilus | homiensis | isoporae |
| Paracoccus | Paracoccus | Paracoccus | Paracoccus | Paracoccus |
| pantotrophus | saliphilus | sanguinis | sediminis | seriniphilus |
| Paracoccus | Paracoccus | Paracoccus | Paracoccus | Paracoccus |
| solventivorans | sp. 228 | sp. J55 | sp. J56 | sp. MKU1 |
| Paracoccus | Paracoccus | Paracoccus | Paracoccus | Paracoccus |
| sp. N5 | sp. PAMC | sp. S4493 | sp. SM22M-07 | sp. TRP |
| | 22219 | | | |
| Paracoccus | Paracoccus | Paracoccus | Paracoccus | Paracoccus |
| sphaerophysae | tibetensis | versutus | yeei | zeaxanthinifaciens |
| Paraferrimonas | Paraferrimonas | Paraferrimonas | Parafilimonas | Parageobacillus |
| sedimenticola | sp. AFRC7-2-1 | sp. CFD7-4-2 | terrae | caldoxylosilyticus |
| Parageobacillus | Parageobacillus | Parageobacillus | Parageobacillus | Paraglaciecola |
| genomosp. 1 | thermantarcticus | thermoglucosidans | toebii | agarilytica |
| Paraglaciecola | Paraglaciecola | Paraglaciecola | Paraglaciecola | Paraliobacillus |
| arctica | polaris | psychrophila | sp. S66 | ryukyuensis |
| Paraliobacillus | Paramaledivibacter | Paramecium | Paramesorhizobium | Paraoerskovia |
| sp. PM-2 | caminithermalis | tetraurelia | deserti | marina |
| Parapedobacter | Parapedobacter | Parapedobacter | Parapedobacter | Paraphaeosphaeria |
| composti | indicus | koreensis | luteus | sporalosa |
| Paraprevotella | Paraprevotella | Pararhizobium | Pararhizobium | Pararhodospirillum |
| clara | xylaniphila | antarcticum | polonicum | photometricum |
| Parasaccharibacter | Parascardovia | Parasporobacterium | Parastagonospora | Parasutterella |
| apium | denticolens | paucivorans | nodorum | excrementihominis |
| Parvibaculum | Parvimonas | Parvimonas | Parvimonas | Parvimonas |
| lavamentivorans | micra | sp. KA00067 | sp. oral | sp. oral |
| | | | taxon 110 | taxon 393 |
| ParvularcUla | Parvularctila | Pasteurella | Pasteurella | Pasteurella |
| bermudensis | oceani | bettyae | dagmatis | multocida |
| Pasteurella | Pasteurella | Pasteur | Patulibacter | Patulibacter |
| skyensis | testudinis | ellaceae | americanus | medicamentivorans |
| | | bacterium 15-036681 | | |
| Patulibacter | Paucibacter | Paucisalibacillus | Paucisalibacillus | Pectobacterium |
| minatonensis | sp. KCTC | globulus | sp. EB02 | atrosepticum |
| | 42545 | | | |
| Pectobacterium | Pectobacterium | Pectobacterium | Pectobacterium | Pectobacterium |
| betavasculorum | carotovorum | parmentieri | sp. SCC3193 | wasabiae |
| Pediococcus | Pediococcus | Pediococcus | Pediococcus | Pediococcus |
| acidilactici | argentinicus | cellicola | claussenii | damnosus |
| Pediococcus | Pediococcus | Pediococcus | Pediococcus | Pediococcus |
| ethanolidurans | inopinatus | parvulus | pentosaceus | stilesii |
| Pedobacter | Pedobacter | Pedobacter | Pedobacter | Pedobacter |
| africanus | agri | antarcticus | arcticus | borealis |
| Pedobacter | Pedobacter | Pedobacter | Pedobacter | Pedobacter |
| caeni | cryoconitis | ginsenosidimutans | glucosidilyticus | hartonius |
| Pedobacter | Pedobacter | Pedobacter | Pedobacter | Pedobacter |
| heparinus | insulae | jeongneungensis | kyungheensis | lusitanus |
| Pedobacter | Pedobacter | Pedobacter | Pedobacter | Pedobacter |
| luteus | nyackensis | oryzae | panaciterrae | psychrophilus |
| Pedobacter | Pedobacter | Pedobacter | Pedobacter | Pedobacter |
| rhizosphaerae | ruber | soli | sp. AJM | sp. BAL39 |
| Pedobacter | Pedobacter | Pedobacter | Pedobacter | Pedobacter |
| sp. BMA | sp. Hv1 | sp. Leaf176 | sp. Leaf1 94 | sp. Leaf216 |
| Pedobacter | Pedobacter | Pedobacter | Pedobacter | Pedobacter |
| sp. Leaf41 | sp. ok626 | sp. PACM 27299 | sp. R20-19 | sp. V48 |
| Pedobacter | Pedobacter | Pedobacter | Pedosphaera | Pelagibaca |
| steynii | suwonensis | terrae | parvula | abyssi |
| Pelagibaca | Pelagibacteraceae | Pelagibacteraceae | Pelagibacteraceae | Pelagibacteraceae |
| bermudensis | bacterium | bacterium | bacterium | bacterium |
| | ETNP-OMZ-SAG-A10 | ETNP-OMZ-SAG-A6 | ETNP-OMZ-SAG-A7 | ETNP-OMZ-SAG-B2 |
| Pelagibacteraceae | Pelagibacteraceae | Pelagibacteraceae | Pelagibacteraceae | Pelagibacterium |
| bacterium | bacterium | bacterium | bacterium | luteolum |
| ETNP-OMZ-SAG-B3 | ETNP-OMZ-SAG-D9 | ETNP-OMZ-SAG-E2 | ETNP-OMZ-SAG-E4 | |
| Pelagicola | Pelagirhabdus | Pelobacter | Pelobacter | Pelobacter |
| litorisediminis | alkalitolerans | acetylenicus | carbinolicus | propionicus |
| Pelobacter | Pelobacter | Pelodictyon | Pelodictyon | Pelomonas |
| seleniigenes | sp. SFB93 | luteolum | phaeoclathratiforme | puraquae |
| Pelomonas | Pelomonas | Pelomonas | Pelomonas | Pelosinus |
| sp. KK5 | sp. Root1217 | sp. Root1237 | sp. Root1444 | fermentans |
| Pelosinus | Pelosinus | Penicillium | Penicillium | Penicillium |
| propionicus | sp. UFO1 | digitatum | expansum | mbens |
| Peptoanaerobacter | Peptoclostridium | Peptoclostridium | Peptococcaceae | Peptococcus |
| stomatis | acidaminophilum | litorale | bacterium CEB3 | niger |
| Peptoniphilus | Peptoniphilus | Peptoniphilus | Peptoniphilus | Peptoniphilus |
| asaccharolyticus | coxii | duerdenii | grossensis | harei |
| Peptoniphilus | Peptoniphilus | Peptoniphilus | Peptoniphilus | Peptoniphilus |
| indolicus | lacrimalis | obesi | phoceensis | rhinitidis |
| Peptoniphilus | Peptoniphilus | Peptoniphilus | Peptoniphilus | Peptoniphilus |
| senegalensis | sp. BV3AC2 | sp. BV3C26 | sp. ChDC B134 | sp. DNF00840 |
| Peptoniphilus | Peptoniphilus | Peptoniphilus | Peptoniphilus | Peptoniphilus |
| sp. EL1 | sp. HMSC062D09 | sp. HMSC075B08 | sp. ING2-D1G | sp. KHD2 |
| Peptoniphilus | Peptoniphilus | Peptoniphilus | Peptoniphilus | Peptoniphilus |
| sp. KHD4 | sp. KHD5 | sp. oral | sp. oral | sp. oral |
| | | taxon 375 | taxon 386 | taxon 836 |
| Peptoniphilus | Peptoniphilus | Peptostreptococcaceae | Peptostreptococcaceae | Peptostreptococcaceae |
| timonensis | urinimassiliensis | bacterium AS15 | bacterium oral taxon | bacterium pGA-8 |
| | | | 113 | |
| Peptostreptococcaceae | Peptostreptococcus | Peptostreptococcus | Peptostreptococcus | Peptostreptococcus |
| bacterium VA2 | anaerobius | russellii | sp. D1 | sp. MV1 |
| Peptostreptococcus | Perkinsus | Perlucidibaca | Perlucidibaca | Persephonella |
| stomatis | marinus | aquatica | piscinae | marina |
| Persephonella sp. | Persephonella | Persicobacter | Persicobacter | Pestalotiopsis |
| IF05-L8 | sp. KM09-Lau-8 | sp. CCB-QB2 | sp. JZB09 | fici |
| Petrimonas | Petrotoga | Phaeoacremonium | Phaeobacter | Phaeobacter |
| mucosa | mobilis | minimum | gallaeciensis | inhibens |
| Phaeobacter | Phaeobacter | Phaeobacter | Phaeobacter | Phaeobacter |
| porticola | sp. 11ANDIMAR09 | sp. 22II1- | sp. CECT | sp. CECT |
| | | 1F12B | 5382 | 7735 |
| Phaeobacter | Phaeobacter | Phaeocystis | Phaeodactylibacter | Phaeodactylum |
| sp. S26 | sp. S60 | globosa | xiamenensis | tricornutum |
| | | virus | | |
| Phaeospirillum | Phaeospirillum | Phanerochaete | Phascolarctobacterium | Phaseolibacter |
| fulvum | molischianum | carnosa | succinatutens | flectens |
| Phaseolus | Phenylobacterium | Phenylobacterium | Phenylobacterium | Phenylobacterium |
| vulgaris | immobile | sp. CCH12-B4 | sp. Root700 | zucineum |
| alphaendornavirus 1 | | | | |
| Phialocephala | Phialophora | Phocaeicola | Phocea | Phoenicibacter |
| scopiformis | attae | abscessus | massiliensis | massiliensis |
| Phormidesmis | Phormidium | Phormidium | Phormidium | Phormidium |
| priestleyi | ambiguum | sp. HE10JO | tenue | willei |
| Photobacterium | Photobacterium | Photobacterium | Photobacterium | Photobacterium |
| angustum | aphoticum | aquae | aquimaris | damselae |
| Photobacterium | Photobacterium | Photobacterium | Photobacterium | Photobacterium |
| gaetbulicola | galatheae | ganghwense | halotolerans | iliopiscarium |
| Photobacterium | Photobacterium | Photobacterium | Photobacterium | Photobacterium |
| jeanii | kishitanii | leiognathi | marinum | phosphoreum |
| Photobacterium | Photobacterium | Photobacterium | Photobacterium | Photobacterium |
| piscicola | profundum | proteolyticum | sanctipauli | sanguinicancri |
| Photobacterium | Photobacterium | Photobacterium | Photobacterium | Photobacterium |
| sp. CECT 9190 | sp. CECT 9192 | sp. H01 100410B | sp. J15 | sp. JCM 19050 |
| Photobacterium | Photobacterium | Photorhabdus | Photorhabdus | Photorhabdus |
| sp. SKA34 | swingsii | asymbiotica | heterorhabditis | luminescens |
| Photorhabdus | Phycicoccus | Phycicoccus | Phycicoccus | Phycicoccus |
| temperata | cremeus | dokdonensis | jejuensis | sp. Root101 |
| Phycicoccus | Phycicoccus | Phycicoccus | Phycicoccus | Phycisphaera |
| sp. Root563 | sp. Soil748 | sp. Soil802 | sp. Soil803 | mikurensis |
| Phycisphaerae | Phycisphaerae | Phycisphaerae | Phycomyces | Phyllobacterium |
| bacterium L21-RPul- | bacterium SM-PulAB- | bacterium ST-PulAB- | blakesleeanus | sp. CL33Tsu |
| D3 | D3 | D4 | | |
| Phyllobacterium | Phyllobacterium | Phyllobacterium | Phyllobacterium | Phytophthora |
| sp. OV277 | sp. UNC302MFCol5.2 | sp. YR531 | sp. YR620 | infestans |
| Phytophthora | Phytophthora | Pichia | Pichia | Pigmentiphaga |
| parasitica | sojae | kudriavzevii | membranifaciens | sp. NML030171 |
| Pigmentiphaga | Pilibacter | Pimelobacter | Pirellula | Piscibacillus |
| sp. NML080357 | termitis | simplex | staleyi | halophilus |
| Piscicoccus | Pisciglobus | Piscirickettsia | Piscirickettsia | Piscirickettsiaceae |
| intestinalis | halotolerans | litoralis | salmonis | bacterium NZ-RLO |
| Planctomicrobium | Planctomyces | Planctomyces | Planctopirus | Planctopirus |
| piriforme | sp. SH- | sp. SH- | limnophila | sp. JC280 |
| | PL14 | PL62 | | |
| Planifilum | Planktomarina | Planktotalea | Planktotalea | Planktothricoides |
| fulgidum | temperata | arctica | frisia | sp. SR001 |
| Planktothrix | Planktothrix | Planktothrix | Planktothrix | Planktothrix |
| agardhii | paucivesiculata | serta | sp. PCC | tepida |
| | | | 11201 | |
| Planobispora | Planococcaceae | Planococcus | Planococcus | Planococcus |
| rosea | bacterium VT-49 | antarcticus | donghaensis | faecalis |
| Planococcus | Planococcus | Planococcus | Planococcus | Planococcus |
| halocryophilus | kocurii | maritimus | massiliensis | plakortidis |
| Planococcus | Planococcus | Planococcus | Planococcus | Planococcus |
| rifietoensis | salinarum | sp. CAU13 | sp. L10.15 | sp. PAMC 21323 |
| Planomicrobium | Planomicrobium | Planomicrobium | Planomonospora | Plantactinospora |
| flavidum | glaciei | okeanokoites | sphaerica | sp. KBS50 |
| Plantibacter | Plantibacter | Plantibacter | Plantibacter | Plantibacter |
| cousiniae | elymi | flavus | sp. H53 | sp. MMLR14 011 |
| Plasmodium | Plasmodium | Plasmodium | Plasmodium | Plasmodium |
| berghei | chabaudi | coatneyi | cynomolgi | falciparum |
| Plasmodium | Plasmodium | Plasmodium | Plasmodium | Plasmodium |
| fragile | gaboni | inui | knowlesi | reichenowi |
| Plasmodium | Plasmodium | Plasmodium | Plautia | Pleomorphobacterium |
| vinckei | vivax | yoelii | stali symbiont | xiamenense |
| Pleomorphomonas | Pleomorphomonas | Plesiocystis | Plesiomonas | Plesiomonas |
| koreensis | oryzae | pacifica | shigelloides | sp. ZOR0011 |
| Pleurocapsa | Pleurocapsa | Pluralibacter | Pneumocystis | Pneumocystis |
| minor | sp. PCC | gergoviae | carinii | jirovecii |
| | 7319 | | | |
| Pneumocystis | Pochonia | Podospora | Poinsettia branch- | Polaribacter |
| murina | chlamydosporia | anserina | inducing phytoplasma | atrinae |
| Polaribacter | Polaribacter | Polaribacter | Polaribacter | Polaribacter |
| dokdonensis | haliotis | irgensii | reichenbachii | sp. BM10 |
| Polaribacter | Polaribacter | Polaribacter | Polaribacter | Polaribacter |
| sp. Hel_I_88 | sp. Hel_I33_49 | sp. HelI_33_78 | sp. HelI_85 | sp. KT 15 |
| Polaribacter | Polaribacter | Polaribacter | Polaribacter | Polaribacter |
| sp. KT25b | sp. MED152 | sp. SA4- | sp. SA4- | sp. WS2- |
| | | 10 | 12 | 14 |
| Polaribacter | Polaromonas | Polaromonas | Polaromonas | Polaromonas |
| vadi | glacialis | jejuensis | naphthalenivorans | sp. A23 |
| Polaromonas | Polaromonas | Polaromonas | Polaromonas | Polaromonas |
| sp. C04 | sp. CF318 | sp. CG9_12 | sp. EUR3 1.2.1 | sp. JS666 |
| Polaromonas | Polaromonas | Polycyclovorans | Polygonum ringspot | Polymorphum |
| sp. OV174 | sp. YR568 | algicola | orthotospovirus | gilvum |
| Polynucleobacter | Polynucleobacter | Polynucleobacter | Polynucleobacter | Polynucleobacter |
| asymbioticus | cosmopolitanus | duraquae | necessarius | sinensis |
| Polynucleobacter | Polynucleobacter | Polynucleobacter | Polynucleobacter | Polynucleobacter |
| sp. MWH-Adler-W8 | sp. MWH-EgelM1-30-B4 | sp. MWH-Feld-100 | sp. MWH-K35W1 | sp. MWH-VicM1 |
| Polynucleobacter | Polynucleobacter | Polynucleobacter | Polynucleobacter | Polysphondylium |
| sp. VK13 | sphagniphilus | wuianus | yangtzensis | pallidum |
| Pontibaca | Pontibacillus | Pontibacillus | Pontibacillus | Pontibacillus |
| methylaminivorans | chungwhensis | halophilus | litoralis | marinus |
| Pontibacillus | Pontibacter | Pontibacter | Pontibacter | Pontibacter |
| yanchengensis | actiniarum | akesuensis | chinhatensis | indicus |
| Pontibacter | Pontibacter | Pontibacter | Pontibacter | Pontibacter |
| korlensis | lucknowensis | roseus | sp. BAB1700 | sp. S10-8 |
| Pontibacter | Ponticaulis | Ponticoccus | Porcine type-C | Porphyrobacter |
| ummariensis | koreensis | sp. SJ5A-1 | oncovirus | colymbi |
| Porphyrobacter | Porphyrobacter | Porphyrobacter | Porphyrobacter | Porphyrobacter |
| cryptus | dokdonensis | donghaensis | mercurialis | neustonensis |
| Porphyrobacter | Porphyrobacter | Porphyrobacter | Porphyrobacter | Porphyrobacter |
| sanguineus | sp. AAP60 | sp. AAP82 | sp. CACIAM 03H1 | sp. HL-46 |
| Porphyrobacter | Porphyrobacter | Porphyromonadaceae | Porphyromonadaceae | Porphyromonadaceae |
| sp. LM 6 | tepidarius | bacterium COT-184 | bacterium FC4 | bacterium H1 |
| | | OH4590 | | |
| Porphyromonadaceae | Porphyromonadaceae | Porphyromonadaceae | Porphyromonadaceae | Porphyromonas |
| bacterium KH3CP3RA | bacterium KH3R12 | bacterium KHP3R9 | bacterium NLAE-zl- | asaccharolytica |
| | | | C104 | |
| Porphyromonas | Porphyromonas | Porphyromonas | Porphyromonas | Porphyromonas |
| bennonis | cangingivalis | canoris | catoniae | circumdentaria |
| Porphyromonas | Porphyromonas | Porphyromonas | Porphyromonas | Porphyromonas |
| crevioricanis | endodontalis | gingivalis | gingivicanis | gulae |
| Porphyromonas | Porphyromonas | Porphyromonas | Porphyromonas | Porphyromonas |
| levii | macacae | somerae | sp. COT-052 OH4946 | sp. COT-108 OH1349 |
| Porphyromonas | Porphyromonas | Porphyromonas | Porphyromonas | Porphyromonas |
| sp. COT-108 OH2963 | sp. COT-239 OH1446 | sp. COT-290 OH3588 | sp. COT-290 OH860 | sp. HMSC065F10 |
| Porphyromonas | Porphyromonas | Porphyromonas | Porphyromonas | Porphyromonas |
| sp. HMSC077F02 | sp. KLE 1280 | sp. oral taxon 278 | sp. oral taxon 279 | uenonis |
| Porticoccus | Poseidonocella | Poseidonocella | Postia placenta | Pragia fontium |
| hydrocarbonoclasticus | pacifica | sedimentorum | | |
| Prauserella | Prauserella | Prevotella off. | Prevotella | Prevotella |
| marina | rugosa | ruminicola Tc2-24 | albensis | amnii |
| Prevotella | Prevotella | Prevotella | Prevotella | Prevotella |
| aurantiaca | baroniae | bergensis | bivia | brevis |
| Prevotella | Prevotella | Prevotella | Prevotella | Prevotella |
| bryantii | buccae | buccalis | conceptionensis | copri |
| Prevotella | Prevotella | Prevotella | Prevotella | Prevotella |
| corporis | dentalis | dentasini | denticola | disiens |
| Prevotella | Prevotella | Prevotella | Prevotella | Prevotella |
| enoeca | falsenii | fusca | histicola | ihumii |
| Prevotella | Prevotella | Prevotella | Prevotella | Prevotella |
| intermedia | jejuni | loescheii | maculosa | marshii |
| Prevotella | Prevotella | Prevotella | Prevotella | Prevotella |
| melaninogenica | micans | multiformis | multisaccharivorax | nanceiensis |
| Prevotella | Prevotella | Prevotella | Prevotella | Prevotella |
| nigrescens | oralis | oris | oryzae | oulorum |
| Prevotella | Prevotella | Prevotella | Prevotella | Prevotella |
| pallens | paludivivens | phocaeensis | pleuritidis | ruminicola |
| Prevotella | Prevotella | Prevotella | Prevotella | Prevotella |
| saccharolytica | salivae | scopos | shahii | sp. 10(H) |
| Prevotella | Prevotella | Prevotella | Prevotella | Prevotella |
| sp. 109 | sp. 885 | sp. AGR2160 | sp. BP1-145 | sp. BP1-148 |
| Prevotella | Prevotella | Prevotella | Prevotella | Prevotella |
| sp. BV3P1 | sp. C561 | sp. DNF00663 | sp. F0091 | sp. FD3004 |
| Prevotella | Prevotella | Prevotella | Prevotella | Prevotella |
| sp. HJM029 | sp. HMSC069G02 | sp. HMSC073D09 | sp. HUN102 | sp. ICM33 |
| Prevotella | Prevotella | Prevotella | Prevotella | Prevotella |
| sp. KH2C16 | sp. KHD1 | sp. khpI | sp. khp7 | sp. lc2012 |
| Prevotella | Prevotella | Prevotella | Prevotella | Prevotella |
| sp. MA2016 | sp. MSX73 | sp. ne3005 | sp. oral | sp. oral |
| | | | taxon 299 | taxon 306 |
| Prevotella | Prevotella | Prevotella | Prevotella | Prevotella |
| sp. oral | sp. oral | sp. oral | sp. P2-180 | sp. P3-120 |
| taxon 317 | taxon 472 | taxon 473 | | |
| Prevotella | Prevotella | Prevotella | Prevotella | Prevotella |
| sp. P3-122 | sp. P3-92 | sp. P4-119 | sp. P4-51 | sp. P4-65 |
| Prevotella | Prevotella | Prevotella | Prevotella | Prevotella |
| sp. P4-67 | sp. P4-76 | sp. P4-98 | sp. P5-108 | sp. P5-119 |
| Prevotella | Prevotella | Prevotella | Prevotella | Prevotella |
| sp. P5-125 | sp. P5-126 | sp. P5-50 | sp. P5-60 | sp. P5-64 |
| Prevotella | Prevotella | Prevotella | Prevotella | Prevotella |
| sp. P5-92 | sp. P6B1 | sp. P6B4 | sp. RM4 | sp. S7 MS 2 |
| Prevotella | Prevotella | Prevotella | Prevotella | Prevotella |
| sp. S7-1-8 | sp. tc2-28 | sp. tf2-5 | stercorea | timonensis |
| Prevotella | Prevotellaceae | Prevotellaceae | Prevotellaceae | Prevotellaceae |
| veroralis | bacterium HUN156 | bacterium KH2P17 | bacterium Marseille- | bacterium MN60 |
| | | | P2826 | |
| Prevotellamassilia | Pricia | Prochlorococcus | Prochlorococcus | Prochlorococcus |
| timonensis | antarctica | marinus | sp. HOT208_60m_{—} | sp. HOT208_60m_{—} |
| | | | 808M21 | 813B04 |
| Prochlorococcus | Prochlorococcus | Prochlorococcus | Prochlorococcus | Prochlorococcus |
| sp. HOT208_60m_{—} | sp. HOT208_60m_{—} | sp. HOT212_60m_{—} | sp. HOT212_60m_{—} | sp. MIT 0601 |
| 813G15 | 813O14 | 824E10 | 826P21 | |
| Prochlorococcus | Prochlorococcus | Prochlorococcus | Prochlorococcus | Prochlorothrix |
| sp. MIT 0604 | sp. MIT 0801 | sp. MIT 1303 | sp. MIT 1306 | hollandica |
| Prolixibacter | Promicromonospora | Promicromonospora | Promicromonosporaceae | Propionibacteriaceae |
| bellariivorans | kroppenstedtii | sukumoe | bacterium W15 | bacterium NML |
| | | | | 020265 |
| Propionibacteriaceae | Propionibacteriaceae | Propionibacteriaceae | Propionibacteriaceae | Propionibacteriaceae |
| bacterium NML | bacterium NML | bacterium NML | bacterium NML | bacterium NML |
| 030167 | 061049 | 090529 | 130396 | 140250 |
| Propionibacteriaceae | Propionibacteriaceae | Propionibacteriaceae | Propionibacterium | Propionibacterium |
| bacterium NML | bacterium NML | bacterium P6A17 | acidifaciens | cyclohexanicum |
| 150081 | 160184 | | | |
| Propionibacterium | Propionibacterium | Propionibacterium | Propionibacterium | Propionibacterium |
| freudenreichii | phage PHL009 | sp. 434-HC2 | sp. HGH0353 | sp. HMSC068C01 |
| Propionibacterium | Propionibacterium | Propionibacterium | Propionibacterium | Propionicicella |
| sp. HMSC078F01 | sp. KPL1844 | sp. KPL2008 | sp. oral taxon 192 | superfundia |
| Propionimicrobium | Propionimicrobium | Propionimicrobium | Propionispira | Propionispira |
| lymphophilum | sp. BV2F7 | sp. Marseille-P3275 | arboris | raffinosivorans |
| Propionispora | Propionispora | Propionivibrio | Prosthecobacter | Prosthecochloris |
| sp. 2/2-37 | vibrioides | dicarboxylicus | debontii | aestuarii |
| Prosthecochloris | Prosthecochloris | Prosthecochloris | Prosthecomicrobium | Proteiniborus |
| sp. CIB 2401 | sp. GSB1 | sp. HL-130-GSB | hirschii | ethanoligenes |
| Proteiniborus | Proteiniclasticum | Proteiniphilum | Proteiniphilum | Proteocatella |
| sp. DW1 | ruminis | acetatigenes | saccharofermentans | sphenisci |
| Proteus | Proteus | Proteus | Proteus | Provencibacterium |
| hauseri | mirabilis | sp. H24 | vulgaris | massiliense |
| Providencia | Providencia | Providencia | Providencia | Providencia |
| alcalifaciens | burhodogranariea | heimbachae | rettgeri | mstigianii |
| Providencia | Providencia | Prune dwarf virus | Pseudacidovorax | Pseudacidovorax |
| sneebia | stuartii | | intermedius | sp. RU35E |
| Pseudaminobacter | Pseudaminobacter | Pseudanabaena | Pseudanabaena | Pseudanabaena |
| manganicus | salicylatoxidans | biceps | sp. PCC 6802 | sp. PCC 7367 |
| Pseudanabaena | Pseudanabaena | Pseudarcicella | Pseudarthrobacter | Pseudarthrobacter |
| sp. ‘Roaring Creek’ | sp. SR411 | hirudinis | chlorophenolicus | equi |
| Pseudarthrobacter | Pseudarthrobacter | Pseudarthrobacter | Pseudoalteromonas | Pseudoalteromonas |
| phenanthrenivorans | siccitolerans | sulfonivorans | agarivorans | aliena |
| Pseudoalteromonas | Pseudoalteromonas | Pseudoalteromonas | Pseudoalteromonas | Pseudoalteromonas |
| arabiensis | atlantica | byunsanensis | citrea | denitrificans |
| Pseudoalteromonas | Pseudoalteromonas | Pseudoalteromonas | Pseudoalteromonas | Pseudoalteromonas |
| elyakovii | espejiana | gelatinilytica | haloplanktis | issachenkonii |
| Pseudoalteromonas | Pseudoalteromonas | Pseudoalteromonas | Pseudoalteromonas | Pseudoalteromonas |
| lipolytica | luteoviolacea | phenolica | piratica | piscicida |
| Pseudoalteromonas | Pseudoalteromonas | Pseudoalteromonas | Pseudoalteromonas | Pseudoalteromonas |
| prydzensis | rubra | ruthenica | shioyasakiensis | sp. 10-33 |
| Pseudoalteromonas | Pseudoalteromonas | Pseudoalteromonas | Pseudoalteromonas | Pseudoalteromonas |
| sp. A601 | sp. BMB | sp. BSi20480 | sp. BSi20495 | sp. BSi20652 |
| Pseudoalteromonas | Pseudoalteromonas | Pseudoalteromonas | Pseudoalteromonas | Pseudoalteromonas |
| sp. EB27 | sp. ECSMB14103 | sp. H103 | sp. H105 | sp. H71 |
| Pseudoalteromonas | Pseudoalteromonas | Pseudoalteromonas | Pseudoalteromonas | Pseudoalteromonas |
| sp. NBT06-2 | sp. P1-26 | sp. P1-30 | sp. P1-9 | sp. R3 |
| Pseudoalteromonas | Pseudoalteromonas | Pseudoalteromonas | Pseudoalteromonas | Pseudoalteromonas |
| sp. SCSIO_11900 | sp. SK20 | sp. SW0106-04 | sp. TB64 | spongiae |
| Pseudoalteromonas | Pseudoalteromonas | Pseudoalteromonas | Pseudoalteromonas | Pseudobacteroides |
| tetraodonis | translucida | tunicata | ulvae | cellulosolvens |
| Pseudobutyrivibrio | Pseudobutyrivibrio | Pseudobutyrivibrio | Pseudobutyrivibrio | Pseudobutyrivibrio |
| ruminis | sp. 49 | sp. ACV-2 | sp. AR14 | sp. C4 |
| Pseudobutyrivibrio | Pseudobutyrivibrio | Pseudobutyrivibrio | Pseudobutyrivibrio | Pseudobutyrivibrio |
| sp. JW11 | sp. LB2011 | sp. MD2005 | sp. NOR37 | sp. OR37 |
| Pseudobutyrivibrio | Pseudobutyrivibrio | Pseudobutyrivibrio | Pseudocercospora | Pseudochrobactrum |
| sp. UC1225 | sp. YE44 | xylanivorans | fifiensis | sp. AO18b |
| Pseudochrobactrum | Pseudoclavibacter | Pseudoclavibacter | Pseudoclavibacter | Pseudoclavibacter |
| sp. B5 | alba | bifida | faecalis | helvolus |
| Pseudoclavibacter | Pseudodonghicola | Pseudoduganella | Pseudoflavonifractor | Pseudoflavonifractor |
| soli | xiamenensis | violaceinigra | capillosus | sp. An176 |
| Pseudoflavonifractor | Pseudoflavonifractor | Pseudoflavonifractor | Pseudoflavonifractor | Pseudoflavonifractor |
| sp. An184 | sp. An187 | sp. An44 | sp. An85 | sp. Marseille-P3106 |
| Pseudoglutamicibacter | Pseudogulbenkiania | Pseudogulbenkiania | Pseudogulbenkiania | Pseudogulbenkiania |
| albus | ferrooxidans | sp. MAI-1 | sp. NH8B | subflava |
| Pseudogymnoascus | Pseudogymnoascus | Pseudohaliea | Pseudohongiella | Pseudohongiella |
| destructans | verrucosus | rubra | acticola | spirulinae |
| Pseudolabrys | Pseudomonas | Pseudomonas | Pseudomonas | Pseudomonas |
| sp. Root1462 | abietaniphila | aeruginosa | aestusnigri | agarici |
| Pseudomonas | Pseudomonas | Pseudomonas | Pseudomonas | Pseudomonas |
| alcaligenes | alcaliphila | alkylphenolica | amygdali | anguilliseptica |
| Pseudomonas | Pseudomonas | Pseudomonas | Pseudomonas | Pseudomonas |
| antarctica | argentinensis | arsenicoxydans | asplenii | asturiensis |
| Pseudomonas | Pseudomonas | Pseudomonas | Pseudomonas | Pseudomonas |
| avellanae | azotifigens | azotoformans | balearica | batumici |
| Pseudomonas | Pseudomonas | Pseudomonas | Pseudomonas | Pseudomonas |
| bauzanensis | benzenivorans | borbori | brassicacearum | caeni |
| Pseudomonas | Pseudomonas | Pseudomonas | Pseudomonas | Pseudomonas |
| canadensis | cannabina | capeferrum | caricapapayae | caspiana |
| Pseudomonas | Pseudomonas | Pseudomonas | Pseudomonas | Pseudomonas |
| cedrina | chloritidismutans | chlororaphis | cichorii | citronellolis |
| Pseudomonas | Pseudomonas | Pseudomonas | Pseudomonas | Pseudomonas |
| coleopterorum | composti | congelans | corrugata | costantinii |
| Pseudomonas | Pseudomonas | Pseudomonas | Pseudomonas | Pseudomonas |
| cremoricolorata | deceptionensis | delhiensis | endophytica | entomophila |
| Pseudomonas | Pseudomonas | Pseudomonas | Pseudomonas | Pseudomonas |
| extremaustralis | extremorientalis | flavescens | flexibilis | floridae |
| Pseudomonas | Pseudomonas | Pseudomonas | Pseudomonas | Pseudomonas |
| fluorescens | formosensis | fragi | frederiksbergensis | fulva |
| Pseudomonas | Pseudomonas | Pseudomonas | Pseudomonas | Pseudomonas |
| fuscovaginae | gessardii | gingeri | graminis | granadensis |
| Pseudomonas | Pseudomonas | Pseudomonas | Pseudomonas | Pseudomonas |
| grimontii | guangdongensis | guguanensis | guineae | helleri |
| Pseudomonas | Pseudomonas | Pseudomonas | Pseudomonas | Pseudomonas |
| hussainii | indica | japonica | jessenii | jinjuensis |
| Pseudomonas | Pseudomonas | Pseudomonas | Pseudomonas | Pseudomonas |
| kilonensis | knackmussii | koreensis | kunmingensis | kuykendallii |
| Pseudomonas | Pseudomonas | Pseudomonas | Pseudomonas | Pseudomonas |
| lactis | libanensis | lini | linyingensis | litoralis |
| Pseudomonas | Pseudomonas | Pseudomonas | Pseudomonas | Pseudomonas |
| lundensis | lutea | luteola | mandelii | marginalis |
| Pseudomonas | Pseudomonas | Pseudomonas | Pseudomonas | Pseudomonas |
| marincola | massiliensis | mediterranea | mendocina | migulae |
| Pseudomonas | Pseudomonas | Pseudomonas | Pseudomonas | Pseudomonas |
| mohnii | monteilii | moorei | moraviensis | mosselii |
| Pseudomonas | Pseudomonas | Pseudomonas | Pseudomonas | Pseudomonas |
| mucidolens | nitroreducens | oleovorans | orientalis | oryzae |
| Pseudomonas | Pseudomonas | Pseudomonas | Pseudomonas | Pseudomonas |
| oryzihabitans | otitidis | pachastrellae | panipatensis | parafulva |
| Pseudomonas | Pseudomonas | Pseudomonas | Pseudomonas | Pseudomonas |
| paralactis | pelagia | peli | plecoglossicida | poae |
| Pseudomonas | Pseudomonas | Pseudomonas | Pseudomonas | Pseudomonas |
| pohangensis | prosekii | protegens | proteolytica | pseudoalcaligenes |
| Pseudomonas | Pseudomonas | Pseudomonas | Pseudomonas | Pseudomonas |
| psychrophila | psychrotolerans | punonensis | putida | reinekei |
| Pseudomonas | Pseudomonas | Pseudomonas | Pseudomonas | Pseudomonas |
| resinovorans | rhizosphaerae | rhodesiae | sabulinigri | sagittaria |
| Pseudomonas | Pseudomonas | Pseudomonas | Pseudomonas | Pseudomonas |
| salegens | salomonii | saponiphila | saudimassiliensis | saudiphocaensis |
| Pseudomonas | Pseudomonas | Pseudomonas | Pseudomonas | Pseudomonas |
| savastanoi | segetis | seleniipraecipitans | sihuiensis | simiae |
| Pseudomonas | Pseudomonas | Pseudomonas | Pseudomonas | Pseudomonas |
| soli | sp. 06C | sp. 1 R | sp. 10- | sp. 10B238 |
| | 126 | 17 | 1B | |
| Pseudomonas | Pseudomonas | Pseudomonas | Pseudomonas | Pseudomonas |
| sp. 11/12A | sp. 1239 | sp. 1D4 | sp. 2(2015) | sp. 21 |
| Pseudomonas | Pseudomonas | Pseudomonas | Pseudomonas | Pseudomonas |
| sp. 25 R | sp. 250J | sp. 31 R | sp. 313 | sp. 37R |
| 14 | | 17 | | 15 |
| Pseudomonas | Pseudomonas | Pseudomonas | Pseudomonas | Pseudomonas |
| sp. 45MFCol3.1 | sp. 5 | sp. 58 R | sp. A214 | sp. A3(2016) |
| | | 3 | | |
| Pseudomonas | Pseudomonas | Pseudomonas | Pseudomonas | Pseudomonas |
| sp. A46 | sp. AAC | sp. ABFPK | sp. Ag1 | sp. Ant30-3 |
| Pseudomonas | Pseudomonas | Pseudomonas | Pseudomonas | Pseudomonas |
| sp. AP19 | sp. AP42 | sp. ARP3 | sp. ASS- | sp. ATCC 13867 |
| | | | 1 | |
| Pseudomonas | Pseudomonas | Pseudomonas | Pseudomonas | Pseudomonas |
| sp. ATCC PTA-122608 | sp. AU12215 | sp. B10 | sp. B10(2017) | sp. B11(2017) |
| Pseudomonas | Pseudomonas | Pseudomonas | Pseudomonas | Pseudomonas |
| sp. B14(2017) | sp. B15(2017) | sp. B22(2017) | sp. B26(2017) | sp. B28(2017) |
| Pseudomonas | Pseudomonas | Pseudomonas | Pseudomonas | Pseudomonas |
| sp. B5 (2017) | sp. B8(2017) | sp. Bc-h | sp. BMS12 | sp. BS- |
| | | | | 2016 |
| Pseudomonas | Pseudomonas | Pseudomonas | Pseudomonas | Pseudomonas |
| sp. BTN1 | sp. C5pp | sp. CBZ- | sp. CC6- | sp. CCA |
| | | 4 | YY-74 | 1 |
| Pseudomonas | Pseudomonas | Pseudomonas | Pseudomonas | Pseudomonas |
| sp. CCOS 191 | sp. CF161 | sp. CFII64 | sp. CHM02 | sp. Choll |
| Pseudomonas | Pseudomonas | Pseudomonas | Pseudomonas | Pseudomonas |
| sp. DRA525 | sp. EGD-AK9 | sp. ENNP23 | sp. EpS/L25 | sp. ES3- |
| | | | | 33 |
| Pseudomonas | Pseudomonas | Pseudomonas | Pseudomonas | Pseudomonas |
| sp. Eur1 | sp. FeS53a | sp. FH1 | sp. FH4 | sp. FSL |
| 9.41 | | | | W5-0203 |
| Pseudomonas | Pseudomonas | Pseudomonas | Pseudomonas | Pseudomonas |
| sp. FSL | sp. G5(2012) | sp. GM102 | sp. GM17 | sp. GM18 |
| W5-0299 | | | | |
| Pseudomonas | Pseudomonas | Pseudomonas | Pseudomonas | Pseudomonas |
| sp. GM21 | sp. GM24 | sp. GM25 | sp. GM30 | sp. GM33 |
| Pseudomonas | Pseudomonas | Pseudomonas | Pseudomonas | Pseudomonas |
| sp. GM41(2012) | sp. GM48 | sp. GM49 | sp. GM50 | sp. GM55 |
| Pseudomonas | Pseudomonas | Pseudomonas | Pseudomonas | Pseudomonas |
| sp. GM60 | sp. GM67 | sp. GM74 | sp. GM78 | sp. GM79 |
| Pseudomonas | Pseudomonas | Pseudomonas | Pseudomonas | Pseudomonas |
| sp. GM80 | sp. GM84 | sp. H1h | sp. HMP271 | sp. HMSC08G10 |
| Pseudomonas | Pseudomonas | Pseudomonas | Pseudomonas | Pseudomonas |
| sp. HMSC11A05 | sp. HPB0071 | sp. IB20 | sp. ICMP 19500 | sp. ICMP 3272 |
| Pseudomonas | Pseudomonas | Pseudomonas | Pseudomonas | Pseudomonas |
| sp. In5 | sp. Irchel 3A18 | sp. Irchel 3A5 | sp. Irchel 3A7 | sp. Irchel 3E13 |
| Pseudomonas | Pseudomonas | Pseudomonas | Pseudomonas | Pseudomonas |
| sp. Irchel 3E19 | sp. Irchel 3E20 | sp. Irchel 3F6 | sp. Irchel 3H3 | sp. Irchel 3H7 |
| Pseudomonas | Pseudomonas | Pseudomonas | Pseudomonas | Pseudomonas |
| sp. Irchel 3H9 | sp. Irchel s3a10 | sp. Irchel s3a18 | sp. Irchel s3b2 | sp. Irchel s3b5 |
| Pseudomonas | Pseudomonas | Pseudomonas | Pseudomonas | Pseudomonas |
| sp. Irchel s3h14 | sp. Irchel s3h17 | sp. K2I15 | sp. KG01 | sp. KK4 |
| Pseudomonas | Pseudomonas | Pseudomonas | Pseudomonas | Pseudomonas |
| sp. LAIL14HWK12:I11 | sp. LAIL14HWK12:I7 | sp. Leaf 127 | sp. Leaf129 | sp. Leaf434 |
| Pseudomonas | Pseudomonas | Pseudomonas | Pseudomonas | Pseudomonas |
| sp. Leaf48 | sp. Leaf58 | sp. LFM046 | sp. LPH1 | sp. Lz4W |
| Pseudomonas | Pseudomonas | Pseudomonas | Pseudomonas | Pseudomonas |
| sp. M1 | sp. M30-35 | sp. M47T1 | sp. MF4836 | sp. MF6394 |
| Pseudomonas | Pseudomonas | Pseudomonas | Pseudomonas | Pseudomonas |
| sp. MF6396 | sp. MIACH | sp. ML96 | sp. MOIL14HWK12:I2 | sp. MRSN12121 |
| Pseudomonas | Pseudomonas | Pseudomonas | Pseudomonas | Pseudomonas |
| sp. MT-1 | sp. NBRC 111119 | sp. NBRC 111123 | sp. NBRC 111124 | sp. NBRC 111127 |
| Pseudomonas | Pseudomonas | Pseudomonas | Pseudomonas | Pseudomonas |
| sp. NBRC 111130 | sp. NBRC 111131 | sp. NBRC 111133 | sp. NBRC 111140 | sp. NBRC 111144 |
| Pseudomonas | Pseudomonas | Pseudomonas | Pseudomonas | Pseudomonas |
| sp. NFACC02 | sp. NFACC04-2 | sp. NFACC13-1 | sp. NFACC19-2 | sp. NFACC25 |
| Pseudomonas | Pseudomonas | Pseudomonas | Pseudomonas | Pseudomonas |
| sp. NFACC39-1 | sp. NFACC42-2 | sp. NFACC45 | sp. NFIX28 | sp. NFPP07 |
| Pseudomonas | Pseudomonas | Pseudomonas | Pseudomonas | Pseudomonas |
| sp. NFPP24 | sp. NFR16 | sp. NS1(2017) | sp. ok266 | sp. Os17 |
| Pseudomonas | Pseudomonas | Pseudomonas | Pseudomonas | Pseudomonas |
| sp. P1.31 | sp. P1.8 | sp. p21 | sp. P482 | sp. P818 |
| Pseudomonas | Pseudomonas | Pseudomonas | Pseudomonas | Pseudomonas |
| sp. P97.38 | sp. PA15(2017) | sp. PA27(2017) | sp. PAMC 25886 | sp. PAMC 26793 |
| Pseudomonas | Pseudomonas | Pseudomonas | Pseudomonas | Pseudomonas |
| sp. Pf153 | sp. PH1b | sp. QTF5 | sp. R37(2017) | sp. R45(2017) |
| Pseudomonas | Pseudomonas | Pseudomonas | Pseudomonas | Pseudomonas |
| sp. R62 | sp. RIT288 | sp. RIT357 | sp. RIT- | sp. RIT- |
| | | | PI-a | PI-q |
| Pseudomonas | Pseudomonas | Pseudomonas | Pseudomonas | Pseudomonas |
| sp. RIT- | sp. RL | sp. Root329 | sp. Root401 | sp. Root562 |
| PI-r | | | | |
| Pseudomonas | Pseudomonas | Pseudomonas | Pseudomonas | Pseudomonas |
| sp. Root569 | sp. Root9 | sp. S1E40 | sp. S3E12 | sp. S-6-2 |
| Pseudomonas | Pseudomonas | Pseudomonas | Pseudomonas | Pseudomonas |
| sp. S9 | sp. SCPG-7 | sp. SHC52 | sp. S1D14000 | sp. Snoq117.2 |
| Pseudomonas | Pseudomonas | Pseudomonas | Pseudomonas | Pseudomonas |
| sp. St29 | sp. StFLB209 | sp. T | sp. TAA207 | sp. TAD18 |
| Pseudomonas | Pseudomonas | Pseudomonas | Pseudomonas | Pseudomonas |
| sp. TCU-HL1 | sp. TJI- | sp. TTU2014-066ASC | sp. TTU2014-080ASC | sp. TTU2014-096BSC |
| | 51 | | | |
| Pseudomonas | Pseudomonas | Pseudomonas | Pseudomonas | Pseudomonas |
| sp. TTU2014-105ASC | sp. URHB0015 | sp. URIL14HWK12:I6 | sp. URMO17WK12:I11 | sp. URMO17WK12:I3 |
| Pseudomonas | Pseudomonas | Pseudomonas | Pseudomonas | Pseudomonas |
| sp. URMO17WK12:I4 | sp. UW4 | sp. VLB120 | sp. W15Feb9B | sp. WCHP16 |
| Pseudomonas | Pseudomonas | Pseudomonas | Pseudomonas | Pseudomonas |
| sp. Z003-0.4C(8344-21) | straminea | stutzeri | synxantha | syringae |
| Pseudomonas syringae | Pseudomonas syringae | Pseudomonas syringae | Pseudomonas | Pseudomonas |
| group genomosp. 3 | group genomosp. 7 | pv. coryli | taeanensis | taetrolens |
| Pseudomonas | Pseudomonas | Pseudomonas | Pseudomonas | Pseudomonas |
| thermotolerans | thivervalensis | tolaasii | toyotomiensis | trivialis |
| Pseudomonas | Pseudomonas | Pseudomonas | Pseudomonas | Pseudomonas |
| umsongensis | vancouverensis | veronii | versuta | viridiflava |
| Pseudomonas | Pseudomonas | Pseudomonas | Pseudomonas | Pseudomonas |
| vranovensis | weihenstephanensis | xanthomarina | xinjiangensis | yamanorum |
| Pseudomonas | Pseudonocardia | Pseudonocardia | Pseudonocardia | Pseudonocardia |
| yangmingensis | acaciae | ammonioxydans | asaccharolytica | autotrophica |
| Pseudonocardia | Pseudonocardia | Pseudonocardia | Pseudonocardia | Pseudonocardia |
| dioxanivorans | oroxyli | sp. AL041005-10 | sp. CNS-004 | sp. CNS-139 |
| Pseudonocardia | Pseudonocardia | Pseudonocardia | Pseudonocardia | Pseudonocardia |
| sp. HH130629-09 | sp. HH130630-07 | sp. MH-G8 | sp. P2 | spinosispora |
| Pseudonocardia | Pseudooceanicola | Pseudooceanicola | Pseudooceanicola | Pseudooceanicola |
| thermophila | atlanticus | batsensis | flagellatus | marinus |
| Pseudooceanicola | Pseudooceanicola | Pseudooctadecabacter | Pseudopedobacter | Pseudopelagicola |
| nanhaiensis | nitratireducens | jejudonensis | saltans | gijangensis |
| Pseudophaeobacter | Pseudophaeobacter | Pseudopropionibacterium | Pseudoramibacter | Pseiidorhizobium |
| arcticus | leonis | propionicum | alactolyticus | pelagicum |
| Pseudorhodobacter | Pseudorhodobacter | Pseudorhodobacter | Pseudorhodobacter | Pseudorhodobacter |
| antarcticus | aquimaris | ferrugineus | psychrotolerans | wandonensis |
| Pseudorhodoferax | Pseudorhodoferax | Pseudorhodoferax | Pseudorhodoplanes | Pseudoruegeria |
| sp. Leaf265 | sp. Leaf267 | sp. Leaf274 | sinuspersici | aquimaris |
| Pseudoruegeria | Pseudoruegeria | Pseudoruegeria | Pseudoruegeria | Pseudoscardovia |
| lutimaris | marinistellae | sabulilitoris | sp. SK021 | radai |
| Pseudoscardovia | Pseudosphingobacterium | Pseudospirillum | Pseudothermotoga | Pseudothermotoga |
| suis | domesticum | japonicum | hypogea | thermarum |
| Pseudovibrio | Pseudovibrio | Pseudovibrio | Pseudovibrio | Pseudovibrio |
| ascidiaceicola | axinellae | denitrificans | hongkongensis | sp. Ab134 |
| Pseudovibrio | Pseudovibrio | Pseudovibrio | Pseudovibrio | Pseudovibrio |
| sp. Ad13 | sp. Ad26 | sp. Ad46 | sp. Ad5 | sp. FO- |
| | | | | BEG1 |
| Pseudovibrio | Pseudovibrio | Pseudovibrio | Pseudovibrio | Pseudovibrio |
| sp. JE062 | sp. POLY-S9 | sp. Tun.PHSC04-5.I4 | sp. WM33 | stylochi |
| Pseudoxanthobacter | Pseudoxanthomonas | Pseudoxanthomonas | Pseudoxanthomonas | Pseudoxanthomonas |
| soli | dokdonensis | indica | mexicana | sp. CF125 |
| Pseudoxanthomonas | Pseudoxanthomonas | Pseudoxanthomonas | Pseudoxanthomonas | Pseudoxanthomonas |
| sp. CF385 | sp. GM95 | sp. GW2 | sp. J31 | sp. J35 |
| Pseudoxanthomonas | Pseudoxanthomonas | Pseudoxanthomonas | Pseudoxanthomonas | Pseudoxanthomonas |
| sp. Root630 | sp. Root65 | sp. YR558 | spadix | suwonensis |
| Pseudozobellia | Pseudozyma | Psychrilyobacter | Psychrobacillus | Psychrobacillus |
| thermophila | hubeiensis | atlanticus | psychrodurans | psychrotolerans |
| Psychrobacillus | Psychrobacillus | Psychrobacillus | Psychrobacter | Psychrobacter |
| sp. FJAT-21963 | sp. OK028 | sp. OK032 | alimentarius | aquaticus |
| Psychrobacter | Psychrobacter | Psychrobacter | Psychrobacter | Psychrobacter |
| cibarius | cryohalolentis | glacincola | lutiphocae | pasteurii |
| Psychrobacter | Psychrobacter | Psychrobacter | Psychrobacter | Psychrobacter |
| phenylpyruvicus | piechaudii | piscatorii | sp. 1501(2011) | sp. AntiMn-1 |
| Psychrobacter | Psychrobacter | Psychrobacter | Psychrobacter | Psychrobacter |
| sp. C 20.9 | sp. DAB_AL32B | sp. DAB_AL43B | sp. G | sp. JB385 |
| Psychrobacter | Psychrobacter | Psychrobacter | Psychrobacter | Psychrobacter |
| sp. P11F6 | sp. P11G3 | sp. P11G5 | sp. PAMC 21119 | sp. PRwf-1 |
| Psychrobacter | Psychrobacter | Psychrobacter | Psychroflexus | Psychroflexus |
| sp. Rd 27.2 | sp. SHUES1 | urativorans | gondwanensis | halocasei |
| Psychroflexus | Psychroflexus | Psychroflexus | Psychroflexus | Psychromonas |
| salarius | sediminis | torquis | tropicus | aquimarina |
| Psychromonas | Psychromonas | Psychromonas | Psychromonas | Psychromonas |
| arctica | hadalis | ingrahamii | ossibalaenae | sp. CDI |
| Psychromonas | Psychromonas | Psychroserpens | Psychroserpens | Psychroserpens |
| sp. CNPT3 | sp. SP041 | burtonensis | damuponensis | jangbogonensis |
| Psychroserpens | Puccinia | Punctularia | Puniceibacterium | Puniceibacterium |
| mesophilus | graminis | strigosozonata | sediminis | sp. IMCC21224 |
| Piirpureocillium | Pusillimonas | Pusillimonas | Pusillimonas | Pustulibacterium |
| lilacinum | noertemannii | sp. T2 | sp. T7-7 | marinum |
| Pyramidobacter | Pyramidobacter | Pyrenophora | Pyrenophora | Pyrinomonas |
| piscolens | sp. C12-8 | teres | tritici- | methylaliphatogenes |
| | | | repentis | |
| Pyrococcus | Pyrococcus | Pyrococcus | Pyrococcus | Pyrococcus |
| abyssi | fiiriosus | horikoshii | kukulkanii | sp. NA2 |
| Pyrococcus | Pyrococcus | Pyrodictium | Pyrolobus | Quadrisphaera |
| sp. ST04 | yayanosii | occultum | fumarii | sp. DSM 44207 |
| Rahnella | Rahnella | Rahnella | Raineyella | Ralstonia |
| aquatilis | sp. WP5 | sp. Y9602 | antarctica | insidiosa |
| Ralstonia | Ralstonia | Ralstonia | Ralstonia | Ralstonia |
| mannitolilytica | pickettii | solanacearum | sp. 25mfcol4.1 | sp. A12 |
| Ralstonia | Ralstonia | Ralstonia | Ralstonia | Ralstonia |
| sp. GA3-3 | sp. MD27 | sp. NFACC01 | sp. PBA | sp. UNC404CL21Col |
| Ralstonia | Ramlibacter | Ramlibacter | Raoultella | Raoultella |
| virus RSL1 | sp. Leaf400 | tataouinensis | ornithinolytica | planticola |
| Raoultella | Rappaport | Rasamsonia | Rathayibacter | Rathayibacter |
| terrigena | israeli | emersonii | sp. Leaf296 | sp. Leaf299 |
| Rathayibacter | Rathayibacter | Rathayibacter | Rathayibacter | Rathayibacter |
| sp. VKM | sp. VKM | tanaceti | toxicus | tritici |
| Ac-2121 | Ac-2630 | | | |
| Reichenbachiella | Reichenbachiella | Reinekea | Renibacterium | Reyranella |
| agariperforans | faecimaris | blandensis | salmoninarum | massiliensis |
| Rheinheimera | Rheinheimera | Rheinheimera | Rheinheimera | Rheinheimera |
| baltica | mesophila | nanhaiensis | pacifica | perlucida |
| Rheinheimera | Rheinheimera | Rheinheimera | Rheinheimera | Rheinheimera |
| salexigens | sp. A13L | sp. EpRS3 | sp. KL1 | sp. SA_1 |
| Rheinheimera | Rhinocladiella | Rhizobacter | Rhizobacter | Rhizobacter |
| texasensis | mackenziei | gummiphilus | sp. OV335 | sp. Root1221 |
| Rhizobacter | Rhizobiales | Rhizobiales | Rhizobiales | Rhizobiales |
| sp. Root404 | bacterium | bacterium | bacterium | bacterium |
| | CCH10-E5 | CCH3-A5 | GAS113 | GAS188 |
| Rhizobiales | Rhizobiales | Rhizobiales | Rhizobium | Rhizobium |
| bacterium GAS191 | bacterium HL-109 | bacterium YIM 77505 | acidisoli | aegyptiacum |
| Rhizobium | Rhizobium | Rhizobium | Rhizobium | Rhizobium |
| aethiopicum | alamii | anhuiense | arenae | bangladeshense |
| Rhizobium | Rhizobium | Rhizobium | Rhizobium | Rhizobium |
| ecuadorense | esperanzae | etli | favelukesii | flavum |
| Rhizobium | Rhizobium | Rhizobium | Rhizobium | Rhizobium |
| freirei | gallicum | giardinii | grahamii | hainanense |
| Rhizobium | Rhizobium | Rhizobium | Rhizobium | Rhizobium |
| laguerreae | leguminosarum | leucaenae | loessense | lusitanum |
| Rhizobium | Rhizobium | Rhizobium | Rhizobium | Rhizobium |
| marinum | mesoamericanum | miluonense | mongolense | multihospitium |
| Rhizobium | Rhizobium | Rhizobium | Rhizobium | Rhizobium |
| nepotum | oryzae | oryziradicis | phaseoli | pusense |
| Rhizobium | Rhizobium | Rhizobium | Rhizobium | Rhizobium |
| rhizosphaerae | selenitireducens | sp. 10195 | sp. 2MFCol3.1 | sp. 42MFCr.l |
| Rhizobium | Rhizobium | Rhizobium | Rhizobium | Rhizobium |
| sp. 58 | sp. 9140 | sp. AAP116 | sp. AAP43 | sp. AC27/96 |
| Rhizobium | Rhizobium | Rhizobium | Rhizobium | Rhizobium |
| sp. AC44/96 | sp. AP16 | sp. BR1 0423 | sp. CCGE 510 | sp. CF048 |
| Rhizobium | Rhizobium | Rhizobium | Rhizobium | Rhizobium |
| sp. CF080 | sp. CF097 | sp. CF122 | sp. CF142 | sp. CF258 |
| Rhizobium | Rhizobium | Rhizobium | Rhizobium | Rhizobium |
| sp. CF394 | sp. C1AT894 | sp. GHKF11 | sp. IBUN | sp. IE4771 |
| Rhizobium | Rhizobium | Rhizobium | Rhizobium | Rhizobium |
| sp. IRBG74 | sp. LC145 | sp. LCM | sp. Leaf155 | sp. Leaf262 |
| | | 4573 | | |
| Rhizobium | Rhizobium | Rhizobium | Rhizobium | Rhizobium |
| sp. Leaf306 | sp. Leaf311 | sp. Leaf321 | sp. Leaf341 | sp. Leaf371 |
| Rhizobium | Rhizobium | Rhizobium | Rhizobium | Rhizobium |
| sp. Leaf384 | sp. Leaf386 | sp. Leaf391 | sp. Leaf453 | sp. LMB-1 |
| Rhizobium | Rhizobium | Rhizobium | Rhizobium | Rhizobium |
| sp. N122 | sp. NFR03 | sp. NFR07 | sp. NFR12 | sp. NT-26 |
| Rhizobium | Rhizobium | Rhizobium | Rhizobium | Rhizobium |
| sp. NXC14 | sp. OK494 | sp. OK665 | sp. P44RR- | sp. PDC82 |
| | | | XXIV | |
| Rhizobium | Rhizobium | Rhizobium | Rhizobium | Rhizobium |
| sp. PDO1- | sp. R339 | sp. R634 | sp. R635 | sp. R693 |
| 076 | | | | |
| Rhizobium | Rhizobium | Rhizobium | Rhizobium | Rhizobium |
| sp. Root1203 | sp. Root1204 | sp. Root1220 | sp. Root149 | sp. Root482 |
| Rhizobium | Rhizobium | Rhizobium | Rhizobium | Rhizobium |
| sp. Root483D2 | sp. Root491 | sp. Root564 | sp. Root708 | sp. RSm-3 |
| Rhizobium | Rhizobium | Rhizobium | Rhizobium | Rhizobium |
| sp. RU33A | sp. RU36D | sp. WYCCWR10014 | sp. YK2 | sp. YR060 |
| Rhizobium | Rhizobium | Rhizobium | Rhizobium | Rhizobium |
| sp. YR295 | sp. YR519 | sp. YR528 | sp. YS-1r | sullae |
| Rhizobium | Rhizobium | Rhizobium | Rhizobium | Rhizobium |
| taibaishanense | tibeticum | tropici | undicola | vignae |
| Rhodanobacter | Rhodanobacter fulvus | Rhodanobacter | Rhodanobacter | Rhodanobacter |
| denitrificans | | glycinis | sp. 115 | sp. B04 |
| Rhodanobacter | Rhodanobacter | Rhodanobacter | Rhodanobacter | Rhodanobacter |
| sp. B05 | sp. C01 | sp. C03 | sp. C05 | sp. C06 |
| Rhodanobacter | Rhodanobacter | Rhodanobacter | Rhodanobacter | Rhodanobacter |
| sp. FW510-R10 | sp. OK091 | sp. OR444 | sp. OR87 | sp. OR92 |
| Rhodanobacter | Rhodanobacter | Rhodanobacter | Rhodanobacter | Rhodanobacter |
| sp. Root179 | sp. Root480 | sp. Root561 | sp. Root627 | sp. Soil772 |
| Rhodanobacter | Rhodanobacter | Rhodobaca | Rhodobacter | Rhodobacter |
| spathiphylli | thiooxydans | barguzinensis | aestuarii | capsulatus |
| Rhodobacter | Rhodobacter | Rhodobacter | Rhodobacter | Rhodobacter |
| johrii | megalophilus | sp. 24- | sp. AKP1 | sp. CCB- |
| | | YEA-8 | | MM2 |
| Rhodobacter | Rhodobacter | Rhodobacter | Rhodobacter | Rhodobacteraceae |
| sp. LPB0142 | sp. SW2 | sphaeroides | vinaykumarii | bacterium CY02 |
| Rhodobacteraceae | Rhodobacteraceae | Rhodobacteraceae | Rhodobacteraceae | Rhodobacteraceae |
| bacterium EhC02 | bacterium HIMB11 | bacterium HTCC2083 | bacterium HTCC2150 | bacterium KLH11 |
| Rhodobacteraceae | Rhodobacteraceae | Rhodobacteraceae | Rhodobacteraceae | Rhodobacteraceae |
| bacterium MCCC | bacterium O3.65 | bacterium PD-2 | bacterium SB2 | bacterium WDS1C4 |
| 1A06712 | | | | |
| Rhodobacteraceae | Rhodobacterales | Rhodobacterales | Rhodoblastus | Rhodocista |
| bacterium WFHF2C18 | bacterium HTCC2255 | bacterium Y4I | acidophilus | sp. M1MtkB3 |
| Rhodococcus | Rhodococcus | Rhodococcus | Rhodococcus | Rhodococcus |
| aetherivorans | coprophilus | corynebacterioides | defluvii | erythropolis |
| Rhodococcus | Rhodococcus | Rhodococcus | Rhodococcus | Rhodococcus |
| fascians | gordoniae | hoagii | imtechensis | jostii |
| Rhodococcus | Rhodococcus | Rhodococcus | Rhodococcus | Rhodococcus |
| koreensis | kroppenstedtii | kunmingensis | kyotonensis | maanshanensis |
| Rhodococcus | Rhodococcus | Rhodococcus | Rhodococcus | Rhodococcus |
| marinonascens | opacus | phenolicus | pyridinivorans | qingshengii |
| Rhodococcus | Rhodococcus | Rhodococcus | Rhodococcus | Rhodococcus |
| rhodnii | rhodochrous | ruber | sp. 008 | sp. 05- |
| | | | | 2255-1e |
| Rhodococcus | Rhodococcus | Rhodococcus | Rhodococcus | Rhodococcus |
| sp. 06- | sp. 06- | sp. 06- | sp. 06- | sp. 06- |
| 1059B-a | 1477-1B | 235-1A | 418-1B | 418-5 |
| Rhodococcus | Rhodococcus | Rhodococcus | Rhodococcus | Rhodococcus |
| sp. 1163 | sp. 1168 | sp. 14- | sp. 14- | sp. 14- |
| | | 1411-2a | 2470-1a | 2470-1b |
| Rhodococcus | Rhodococcus | Rhodococcus | Rhodococcus | Rhodococcus |
| sp. 14- | sp. 14- | sp. 15- | sp. 15- | sp. 164Chir2E |
| 2483-1-2 | 2496-1d | 649-2-2 | 725-2-2b | |
| Rhodococcus | Rhodococcus | Rhodococcus | Rhodococcus | Rhodococcus |
| sp. 311R | sp. 4J2A2 | sp. 66b | sp. 852002- | sp. AD45 |
| | | | 51564 SCH6189132-a | |
| Rhodococcus | Rhodococcus | Rhodococcus | Rhodococcus | Rhodococcus |
| sp. ADH | sp. ARP2 | sp. A W25M09 | sp. BH4 | sp. BGPNP1 |
| Rhodococcus | Rhodococcus | Rhodococcus | Rhodococcus | Rhodococcus |
| sp. Chr-9 | sp. CUA- | sp. EPR- | sp. JVH1 | sp. KB6 |
| | 806 | 157 | | |
| Rhodococcus | Rhodococcus | Rhodococcus | Rhodococcus | Rhodococcus |
| sp. Leaf278 | sp. MEB064 | sp. MTM3W5.2 | sp. NCIMB 12038 | sp. OK302 |
| Rhodococcus | Rhodococcus | Rhodococcus | Rhodococcus | Rhodococcus |
| sp. R1101 | sp. RD6.2 | sp. RS1C4 | sp. SC4 | sp. UNC23MFCrub1.1 |
| Rhodococcus | Rhodococcus | Rhodococcus | Rhodococcus | Rhodococcus |
| sp. UNC363MFTsu5.1 | sp. WB1 | sp. WMMA185 | sp. YH3- | triatomae |
| | | | 3 | |
| Rhodococcus | Rhodococcus | Rhodococcus | Rhodococcus | Rhodocyclaceae |
| tukisamuensis | wratislaviensis | yunnanensis | zopfii | bacterium Paddy-1 |
| Rhodoferax | Rhodoferax | Rhodoferax | Rhodoferax | Rhodoferax |
| antarcticus | fermentans | ferrireducens | saidenbachensis | sp. DCY110 |
| Rhodoferax | Rhodohalobacter | Rhodoluna | Rhodomicrobium | Rhodomicrobium |
| sp. TH121 | halophilus | lacicola | udaipurense | vannielii |
| Rhodopirellula | Rhodopirellula | Rhodopirellula | Rhodopirellula | Rhodopirellula |
| baltica | europaea | islandica | maiorica | sallentina |
| Rhodopirellula | Rhodopirellula | Rhodoplanes | Rhodoplanes | Rhodopseudomonas |
| sp. MGV | sp. SWK7 | sp. JGI | sp. Z2- | palustris |
| | | PP 4-B12 | YC6860 | |
| Rhodopseudomonas | Rhodopseudomonas | Rhodopseudomonas | Rhodospira trueperi | Rhodospirillaceae |
| pseudopalustris | sp. AAP120 | sp. B29 | | bacterium CCH5-H10 |
| Rhodospirillales | Rhodospirillales | Rhodospirillales | Rhodospirillum | Rhodospirillum |
| bacterium B29T1 | bacterium URHD0017 | bacterium URHD0088 | centenum | rubrum |
| Rhodothermaceae | Rhodothermus | Rhodothermus | Rhodotorala | Rhodotorala |
| bacterium RA | marinus | profundi | graminis | toraloides |
| Rhodovibrio | Rhodovulum | Rhodovulum | Rhodovulum | Rhodovulum |
| salinarum | sp. ES.010 | sp. MB263 | sp. NI22 | sp. P5 |
| Rhodovulum | Rhodovulum | Richelia | Rickettsia | Rickettsia |
| sp. PH10 | sulfidophilum | intracellularis | aeschlimannii | akari |
| Rickettsia | Rickettsia | Rickettsia | Rickettsia | Rickettsia |
| amblyommatis | asembonensis | australis | bellii | canadensis |
| Rickettsia | Rickettsia | Rickettsia | Rickettsia | Rickettsia |
| conorii | endosymbiont of | felis | gravesii | helvetica |
| | Culicoides newsteadi | | | |
| Rickettsia | Rickettsia | Rickettsia | Rickettsia | Rickettsia |
| honei | hoogstraalii | massiliae | montanensis | philipii |
| Rickettsia | Rickettsia | Rickettsia | Rickettsia | Rickettsia |
| raoultii | rhipicephali | rickettsii | sibirica | slovaca |
| Rickettsia | Rickettsia | Rickettsiales bacterium | Rickettsiella | Riemerella |
| sp. wb | typhi | Ac37b | grylli | anatipestifer |
| Riemerella | Riemerella | Rikenella | Risungbinella | Rivularia |
| columbina | columbipharyngis | microfusus | massiliensis | sp. PCC |
| | | | | 7116 |
| Robiginitalea | Robiginitalea | Robiginitalea | Robiginitomaculum | Robinsoniella |
| biformata | myxolifaciens | sp. O458 | antarcticum | peoriensis |
| Robinsoniella | Rodentibacter | Rodentibacter | Rodentibacter | Rodentibacter |
| sp. KNHs210 | genomosp. 1 | genomosp. 2 | heidelbergensis | heylii |
| Rodentibacter | Rodentibacter | Rodentibacter | Rodentibacter | Rodentibacter |
| mrazii | myodis | pneumotropicus | ranis | ratti |
| Rodentibacter | Rodentibacter | Romboutsia | Romboutsia | Romboutsia |
| sp. Ppn85 | trehalosifermentans | lituseburensis | sp. CCRI- | sp. CCRI- |
| | | | 19649 | 22766 |
| Romboutsia | Romboutsia | Roseateles | Roseateles | Roseateles |
| sp. MT17 | timonensis | aquatilis | depolymerans | sp. YR242 |
| Roseateles | Roseburia | Roseburia | Roseburia | Roseburia |
| terrae | faecis | hominis | intestinalis | inulinivorans |
| Roseburia | Roseburia | Roseibaca | Roseibacterium | Roseibium |
| sp. 499 | sp. 831b | calidilacus | elongation | sp. TrichSKD4 |
| Roseicitreum | Roseiflexus | Roseiflexus | Roseimaritima | Roseinatronobacter |
| antarcticum | castenholzii | sp. RS-1 | ulvae | thiooxidans |
| Roseisalinus | Roseivirga | Roseivirga | Roseivirga | Roseivirga |
| antarcticus | echinicomitans | ehrenbergii | seohaensis | spongicola |
| Roseivivax | Roseivivax | Roseivivax | Roseivivax | Roseivivax |
| atlanticus | halodurans | halotolerans | isoporae | jejudonensis |
| Roseivivax | Roseivivax | Roseivivax | Rosenbergiella | Roseobacter |
| lentus | marinus | roseus | nectarea | denitrificans |
| Roseobacter | Roseobacter | Roseobacter | Roseobacter | Roseobacter |
| litoralis | sp. AzwK- | sp. CCS2 | sp. GA1101 | sp. MED193 |
| | 3b | | | |
| Roseobacter | Roseomonas | Roseomonas | Roseomonas | Roseomonas |
| sp. SK209-2-6 | aerilata | cervicalis | gilardii | mucosa |
| Roseomonas | Roseomonas | Roseomonas | Roseomonas | Roseospirillum |
| rosea | sp. M3 | sp. TAS13 | stagni | parvum |
| Roseovarius | Roseovarius | Roseovarius | Roseovarius | Roseovarius |
| aestuarii | albus | atlanticus | azorensis | gaetbuli |
| Roseovarius | Roseovarius | Roseovarius | Roseovarius | Roseovarius |
| halotolerans | indicus | lutimaris | marisflavi | mucosus |
| Roseovarius | Roseovarius | Roseovarius | Roseovarius | Roseovarius |
| nanhaiticus | nubinhibens | pacificus | sp. 217 | sp. 22II1- |
| | | | | 1F6A |
| Roseovarius | Roseovarius | Roseovarius | Roseovarius | Rothia |
| sp. A-2 | sp. MCTG156(2b) | sp. TM1035 | tolerans | aeria |
| Rothia | Rothia | Rothia | Rothia | Rothia |
| dentocariosa | mucilaginosa | nasimurium | sp. HMSC036D11 | sp. HMSC058E10 |
| Rothia | Rothia | Rothia | Rothia | Rothia |
| sp. HMSC061C12 | sp. HMSC061D12 | sp. HMSC061E04 | sp. HMSC062F03 | sp. HMSC062H08 |
| Rothia | Rothia | Rothia | Rothia | Rothia |
| sp. HMSC064D08 | sp. HMSC064F07 | sp. HMSC065B04 | sp. HMSC065C03 | sp. HMSC065C12 |
| Rothia | Rothia | Rothia | Rothia | Rothia |
| sp. HMSC065D02 | sp. HMSC065D09 | sp. HMSC065G12 | sp. HMSC066G02 | sp. HMSC066G07 |
| Rothia | Rothia | Rothia | Rothia | Rothia |
| sp. HMSC067H10 | sp. HMSC068E02 | sp. HMSC068F09 | sp. HMSC069C01 | sp. HMSC069C04 |
| Rothia | Rothia | Rothia | Rothia | Rothia |
| sp. HMSC069C10 | sp. HMSC069D01 | sp. HMSC071B01 | sp. HMSC071C12 | sp. HMSC071F11 |
| Rothia | Rothia | Rothia | Rothia | Rothia |
| sp. HMSC072B03 | sp. HMSC072B04 | sp. HMSC072E10 | sp. HMSC073B08 | sp. HMSC075F09 |
| Rothia | Rothia | Rothia | Rothia | Rothia |
| sp. HMSC076D04 | sp. HMSC078H08 | sp. HMSC08A08 | sp. ND6WE1A | sp. Olga |
| Rouxiella | Rouxiella | Rouxiella | Ruania albidiflava | Ruaniaceae bacterium |
| badensis | chamberiensis | silvae | | KH17 |
| Rubellimicrobium | Rubellimicrobium | Rubeoparvulum | Rubidibacter | Rubinisphaera |
| mesophilum | thermophilum | massiliense | lacunae | brasiliensis |
| Rubricoccus | Rubrimonas | Rubripirellula | Rubritalea | Rubritepida |
| marinus | cliftonensis | obstinata | marina | flocculans |
| Rubrivirga | Rubrivirga | Rubrivivax | Rubrivivax | Rubrobacter |
| marina | sp. SAORIC476 | benzoatilyticus | gelatinosus | aplysinae |
| Rubrobacter | Rubrobacter | Rudaea | Rudanella | Ruegeria |
| radiotolerans | xylanophilus | cellulosilytica | lutea | arenilitoris |
| Ruegeria | Ruegeria | Ruegeria | Ruegeria | Ruegeria |
| atlantica | conchae | halocynthiae | lacuscaerulensis | marina |
| Ruegeria | Ruegeria | Ruegeria | Ruegeria | Ruegeria |
| meonggei | mobilis | pomeroyi | sp. 6PALISEP08 | sp. ANG-R |
| Ruegeria | Ruegeria | Ruegeria | Ruegeria | Ruegeria |
| sp. ANG-S4 | sp. CECT | sp. PBVC088 | sp. TM1040 | sp. ZGT108 |
| | 5091 | | | |
| Ruegeria | Rufibacter | Rufibacter | Rufibacter | Rufibacter |
| sp. ZGT118 | roseus | ruber | sp. DG15C | sp. DG31D |
| Rufibacter | Rugamonas | Ruminiclostridium | Ruminobacter | Ruminobacter |
| tibetensis | rubra | thermocellum | amylophilus | sp. RM87 |
| Ruminococcaceae | Ruminococcaceae | Ruminococcaceae | Ruminococcaceae | Ruminococcaceae |
| bacterium | bacterium AB4001 | bacterium AE2021 | bacterium CPB6 | bacterium cv2 |
| Ruminococcaceae | Ruminococcaceae | Ruminococcaceae | Ruminococcaceae | Ruminococcaceae |
| bacterium D16 | bacterium D5 | bacterium FB2012 | bacterium KH2T8 | bacterium Marseille- |
| | | | | P2935 |
| Ruminococcaceae | Ruminococcaceae | Ruminococcaceae | Ruminococcus | Ruminococcus |
| bacterium P7 | bacterium YAD3003 | bacterium YRB3002 | albus | bicirculans |
| Ruminococcus | Ruminococcus | Ruminococcus | Ruminococcus | Ruminococcus |
| bromii | callidus | champanellensis | faecis | flavefaciens |
| Ruminococcus | Ruminococcus | Ruminococcus | Ruminococcus | Ruminococcus |
| gauvreauii | lactaris | sp. 5_1_39BFAA | sp. AT10 | sp. DSM 100440 |
| Ruminococcus | Ruminococcus | Ruminococcus | Ruminococcus | Ruminococcus |
| sp. FC2018 | sp. HUN007 | sp. JC304 | sp. NK3A76 | sp. YE71 |
| Ruminococcus | Rummeliibacillus | Runella | Runella | Runella |
| sp. YE78 | stabekisii | limosa | slithyformis | zeae |
| Ruthenibacterium | Saccharibacillus | Saccharibacillus | Saccharibacillus | Saccharibacillus |
| lactatiformans | kuerlensis | sacchari | sp. O16 | sp. O23 |
| Saccharibacter | Saccharibacter | Saccharibacter | Saccharibacter | Saccharicrinis |
| floricola | sp. 3.A.1 | sp. AM169 | sp. M18 | fermentans |
| Saccharomonospora | Saccharomonospora | Saccharomonospora | Saccharomonospora | Saccharomonospora |
| azurea | cyanea | glauca | halophila | marina |
| Saccharomonospora | Saccharomonospora | Saccharomonospora | Saccharomonospora | Saccharomonospora |
| paurometabolica | saliphila | sp. CNQ490 | sp. CUA-673 | viridis |
| Saccharomonospora | Saccharomyces | Saccharomyces | Saccharophagus | Saccharopolyspora |
| xinjiangensis | cerevisiae | eubayanus | degradans | antimicrobica |
| Saccharopolyspora | Saccharopolyspora | Saccharopolyspora | Saccharopolyspora | Saccharopolyspora |
| erythraea | flava | jiangxiensis | rectivirgula | shandongensis |
| Saccharopolyspora | Saccharospirillum | Saccharothrix | Saccharothrix | Saccharothrix |
| spinosa | impatiens | espanaensis | sp. ALI- | sp. CB00851 |
| | | | 22-I | |
| Saccharothrix | Saccharothrix | Saccharothrix | Saccharothrix | Sagittula |
| sp. NRRL B-16314 | sp. NRRL B-16348 | sp. ST- | syringae | stellata |
| | | 888 | | |
| Saitoella | Salegentibacter | Salegentibacter | Salegentibacter | Salegentibacter |
| complicata | agarivorans | echinorum | flavus | holothuriorum |
| Salegentibacter | Salegentibacter | Salegentibacter | Salegentibacter | Salegentibacter |
| mishustinae | salarius | salegens | salinarum | sp. Hel_I_6 |
| Salegentibacter | Salibacterium | Salibacterium | Salimicrobium | Salimicrobium |
| sp. K5023 | halotolerans | qingdaonense | album | flavidum |
| Salimicrobium | Salimicrobium | Salimicrobium | Salinarchaeum | Salinarimonas |
| halophilum | jeotgali | salexigens | sp. Harcht-Bsk1 | rosea |
| Salinibacillus | Salinibacter | Salinibacterium | Salinicoccus | Salinicoccus |
| kushneri | ruber | sp. PAMC 21357 | albus | alkaliphilus |
| Salinicoccus | Salinicoccus | Salinicoccus | Salinicoccus | Salinicoccus |
| carnicancri | halodurans | luteus | qingdaonensis | roseus |
| Salinicoccus | Salinicoccus | Salinicola | Salinicola | Salinicola |
| sediminis | sp. YB14-2 | salarius | socius | sp. MH3R3-1 |
| Salinicola | Salinihabitans | Salinimicrobium | Salinimicrobium | Salinimicrobium |
| sp. MIT1003 | flavidus | catena | terrae | xinjiangense |
| Salinimonas | Salinisphaera | Salinisphaera | Salinispira | Salinispora |
| chungwhensis | hydrothermalis | shabanensis | pacifica | arenicola |
| Salinispora | Salinispora | Salinivibrio | Salinivibrio | Salinivibrio |
| paciflca | tropica | sharmensis | socompensis | sp. BNH |
| Salinivibrio | Salinivibrio | Salinivibrio | Salinivibrio | Salinivibrio |
| sp. DV | sp. ES.052 | sp. IB560 | sp. IB563 | sp. IB574 |
| Salinivibrio | Salinivibrio | Salinivibrio | Salinivibrio | Salinivibrio |
| sp. IB872 | sp. IC317 | sp. MA351 | sp. MA427 | sp. MA607 |
| Salinivibrio | Salinivibrio | Salinivibrio | Salinivirga | Salipaludibacillus |
| sp. ML290 | sp. ML318 | sp. ML323 | cyanobacteriivorans | agaradhaerens |
| Salipaludibacillus | Salipiger | Salisaeta | Salisediminibacterium | Salmonella |
| aurantiacus | mucosus | longa | haloalkalitolerans | bongori |
| Salmonella | Salsuginibacillus | Sandaracinus | Sandarakinorhabdus | Sandarakinorhabdus |
| enterica | kocurii | amylolyticus | limnophila | sp. AAP62 |
| Sandarakinorhabdus | Sanguibacter | Sanguibacter | Sanguibacter | Sanguibacter |
| sp. TH057 | gelidistatuariae | keddieii | marinus | sp. Leaf1 |
| Sanguibacter | Sanguibacteroides | Saprolegnia | Saprolegnia | Saprospira |
| saurezii | justesenii | diclina | parasitica | grandis |
| SAR116 cluster alpha | Sarcina | Scurdovia | Scurdovia | Scedosporium |
| proteobacterium | sp. DSM 11001 | inopinata | wiggsiae | apiospermum |
| H1MB100 | | | | |
| Scheffersomyces | Schizophyllum | Schizosaccharomyces | Schizosaccharomyces | Schizosaccharomyces |
| stipitis | commune | cryophilus | japonicus | octosporus |
| Schizosaccharomyces | Schleiferia | Schlesneria | Schwartzia | Sciscionella |
| pombe | thermophilu | puludicolu | succinivorans | murina |
| Sciscionella | Sclerotinia | Scytonema | Scytonema | Scytonema |
| sp. SE31 | sclerotiorum | hofmannii | millei | sp. HK-05 |
| Scytonema | Sebaldella | secondary | secondary | secondary |
| tolypothrichoides | termitidis | endosymbiont of | endosymbiont of | endosymbiont of |
| | | Ctenarytaina | Heteropsylla | Trabutina |
| | | eucalypti | cubana | mannipara |
| Sedimentibacter | Sedimentibacter | Sedimenticola | Sedimenticola | Sedimentitalea |
| sp. B4 | sp. SX930 | selenatireducens | thiotaarini | nanhaiensis |
| Sediminibacillus | Sediminibacillus | Sediminibacillus | Sediminibacter | Sediminibacterium |
| albus | halophilus | massiliensis | sp. Hel_I_10 | ginsengisoli |
| Sediminibacterium | Sediminibacterium | Sediminicola | Sediminimonas | Sediminispirochaeta |
| salmoneum | sp. C3 | sp. YIK13 | qiohouensis | bajacaliforniensis |
| Sediminispirochaeta | Segetibacter | Segniliparus | Segniliparus | Seinonella |
| smaragdinae | koreensis | rotundus | rugosus | peptonophila |
| Selenihalanaerobacter | Selenomonas | Selenomonas | Selenomonas | Selenomonas |
| shriftii | artemidis | bovis | flueggei | infelix |
| Selenomonas | Selenomonas | Selenomonas | Selenomonas | Selenomonas |
| noxiu | ruminuntium | sp. AE3005 | sp. CM52 | sp. F0473 |
| Selenomonas | Selenomonas | Selenomonas | Selenomonas | Selenomonas |
| sp. FC4001 | sp. FOBRC6 | sp. FOBRC9 | sp. ND2010 | sp. oral |
| | | | | taxon 126 |
| Selenomonas | Selenomonas | Selenomonas | Selenomonas | Selenomonas |
| sp. oral | sp. oral | sp. oral | sp. oral | sp. oral |
| taxon 136 | taxon 137 | taxon 138 | taxon 149 | taxon 478 |
| Selenomonas | Selenomonas | Selenomonas | Sellimonus | Senegaluimassilia |
| sp. oral | sp. oral | sputigena | intestinalis | anaerobia |
| taxon 892 | taxon 920 | | | |
| Seonamhaeicola | Serinicoccus | Serinicoccus | Serinicoccus | Serinicoccus |
| sp. S2-3 | chungangensis | marinus | profundi | sp. CNJ- |
| | | | | 927 |
| Serinicoccus | Serpula | Serratia | Serratia | Serratia |
| sp. JLT9 | lucrymuns | ficaria | fonticola | grimesii |
| Serratia | Serratia | Serratia | Serratia | Serratia |
| liquiefaciens | marcescens | nematodiphila | odorifera | oryzae |
| Serratia | Serratia | Serratia | Serratia | Serratia |
| plymuthica | proteamaculans | rubidaea | sp. 14-2641 | sp. 506_PEND |
| Serratia | Serratia | Serratia | Serratia | Serratia |
| sp. Ag1 | sp. ATCC | sp. C-1 | sp. DD3 | sp. FS14 |
| | 39006 | | | |
| Serratia | Serratia | Serratia | Serratia | Serratia |
| sp. HMSC15F11 | sp. ISTD04 | sp. Leaf50 | sp. Leaf51 | sp. M24T3 |
| Serratia | Serratia | Serratia | Serratia | Serratia |
| sp. S4 | sp. SCBI | sp. TEL | sp. YD25 | symbiotica |
| Setosphaeria | Sharpea | Shewanella | Shewanella | Shewanella |
| turcica | azabuensis | algae | amazonensis | bultica |
| Shewanella | Shewanella | Shewanella | Shewanella | Shewanella |
| benthica | bicestrii | colwelliana | decolorationis | denitrificans |
| Shewanella | Shewanella | Shewanella | Shewanella | Shewanella |
| frigidimarina | halifaxensis | haliotis | japonica | loihica |
| Shewanella | Shewanella | Shewanella | Shewanella | Shewanella |
| mangrovi | marina | morhuae | oneidensis | pealeana |
| Shewanella | Shewanella | Shewanella | Shewanella | Shewanella |
| piezotolerans | psychrophila | putrefaciens | sediminis | sp. 38A_GOM-205m |
| Shewanella | Shewanella | Shewanella | Shewanella | Shewanella |
| sp. Alg231_23 | sp. ANA-3 | sp. cp20 | sp. ECSMB14101 | sp. FDAARGOS_354 |
| Shewanella | Shewanella | Shewanella | Shewanella | Shewanella |
| sp. HN-41 | sp. MR-4 | sp. MR-7 | sp. P1-14-1 | sp. POL2 |
| Shewanella | Shewanella | Shewanella | Shewanella | Shewanella |
| sp. Sh95 | sp. UCD- | sp. UCD- | sp. UCD- | sp. ZOR0012 |
| | FRSSP16 17 | KL12 | KL21 | |
| Shewanella | Shewanella | Shewanella | Shewanella | Shigella |
| violacea | waksmanii | woodyi | xiamenensis | boydii |
| Shigella | Shigella | Shigella | Shigella | Shigella |
| dysenteriae | flexneri | sonnei | sp. FC1172 | sp. FC130 |
| Shigella | Shigella | Shigella | Shigella | Shigella |
| sp. FC1708 | sp. FC2045 | sp. FC2175 | sp. FC2710 | sp. FC2928 |
| Shigella | Shigella | Shigella | Shimazuella | Shimia |
| sp. FC569 | sp. PAMC | sp. SF-2015 | kribbensis | haliotis |
| | 28760 | | | |
| Shimia | Shimia | Shimwellia | Shinella | Shinella |
| marina | sp. SK013 | blattae | sp. DD12 | sp. HZN7 |
| Shuttleworthia | Shuttleworthia | Siansivirga | Siccationidurans | Siccibacter |
| satelles | sp. MSX8B | zeaxanthinifaciens | arizonensis | colletis |
| Sideroxydans | Silanimonas | Silicibacter | Silvibacterium | Simiduia |
| lithotrophicus | lenta | sp. TrichCH4B | bohemicum | agarivorans |
| Simkania | Simonsiella | Simplicispira | Singulisphaera | Singulisphaera |
| negevensis | muelleri | psychrophila | acidiphila | sp. GP187 |
| Sinomicrobium | Sinomonas | Sinomonas | Sinomonas | Sinorhizobium |
| oceani | atrocyanea | humi | mesophila | americanum |
| Sinorhizobium | Sinorhizobium | Sinorhizobium | Sinorhizobium | Sinorhizobium |
| arboris | fredii | medicae | meliloti | saheli |
| Sinorhizobium | Sinorhizobium | Sinorhizobium | Sinorhizobium | Sinorhizobium |
| sp. A49 | sp. CCBAU 05631 | sp. GL28 | sp. GW3 | sp. NFACC03 |
| Sinorhizobium | Sinorhizobium | Sinorhizobium | Sinosporangium | Siphonobacter |
| sp. PC2 | sp. RAC02 | sp. Sb3 | album | aquaeclarae |
| Siphonobacter | Skermanella | Skermanella | Skermania | Slackia |
| sp. BAB-5385 | aerolata | stibiiresistens | piniformis | exigua |
| Slackia | Slackia | Slackia | Smaragdicoccus | Sneathia |
| heliotrinireducens | piriformis | sp. CM382 | niigatensis | amnii |
| Sneathia | Sneathiella | Snodgrassella | Snodgrassella | Snodgrassella |
| sanguinegens | glossodoripedis | alvi | sp. CFCC 13594 | sp. R- |
| | | | | 53583 |
| Sodalis | Sodalis | Solemya | Solemya | Solemya velesiana gill |
| glossinidius | praecaptivus | elarraichensis | pervernicosa | symbiont |
| | | gill symbiont | gill symbiont | |
| Solemya velum gill | Solibacillus | Solibacillus | Solibacillus | Solimonas |
| symbiont | isronensis | kalamii | Silvestris | aquatica |
| Solimonas | Solimonas | Solimonas | Solirubrobacter | Solirubrobacter |
| flava | soli | variicoloris | soli | sp. URHD0082 |
| Solirubrobacterales | Solimbrum | Solitalea | Solobacterium | Soonwooa |
| bacterium URHD0059 | puertoriconensis | canadensis | moorei | buanensis |
| Sorangium | Sordaria | Spathaspora | Sphaerobacter | Sphaerochaeta |
| cellulosum | macrospora | passalidarum | thermophilus | coccoides |
| Sphaerochaeta | Sphaerochaeta | Sphaerotilus | Sphaerulina | Sphingobacteriaceae |
| globosa | pleomorpha | natans | musiva | bacterium DW12 |
| Sphingobacteriaceae | Sphingobacteriales | Sphingobacteriales | Sphingobacterium | Sphingobacterium |
| bacterium GW460-11- | bacterium TSM_CSM | bacterium TSM_CSS | cellulitidis | deserti |
| 11-14-LB5 | | | | |
| Sphingobacterium | Sphingobacterium | Sphingobacterium | Sphingobacterium | Sphingobacterium |
| mizutaii | nematocida | paucimobilis | psychroaquaticum | sp. 21 |
| Sphingobacterium | Sphingobacterium | Sphingobacterium | Sphingobacterium | Sphingobacterium |
| sp. Ag1 | sp. B29 | sp. CFCC 11742 | sp. CZ-UAM | sp. G1-14 |
| Sphingobacterium | Sphingobacterium | Sphingobacterium | Sphingobacterium | Sphingobacterium |
| sp. HMSC13C05 | sp. UTKGP-BTPF85 | sp. JB170 | sp. ML3W | sp. PM2-P1-29 |
| Sphingobacterium | Sphingobacterium | Sphingobacterium | Sphingobacterium | Sphingobium |
| sp. T2 | spiritivorum | thalpophilum | wenxiniae | abikonense |
| Sphingobium | Sphingobium | Sphingobium | Sphingobium | Sphingobium |
| amiense | baderi | chlorophenolicum | chungbukense | cloacae |
| Sphingobium | Sphingobium | Sphingobium | Sphingobium | Sphingobium |
| czechense | faniae | herbicidovorans | japonicum | lactosutens |
| Sphingobium | Sphingobium | Sphingobium | Sphingobium | Sphingobium |
| phenoxybenzoativorans | quisquiliarum | sp. Ant17 | sp. AP49 | sp. AP50 |
| Sphingobium | Sphingobium | Sphingobium | Sphingobium | Sphingobium |
| sp. ba1 | sp. C100 | sp. CCH11-B1 | sp. DC-2 | sp. EP60837 |
| Sphingobium | Sphingobium | Sphingobium | Sphingobium | Sphingobium |
| sp. GW456-12-10-14- | sp. HDIP04 | sp. Leaf26 | sp. MI1205 | sp. Ndbn- |
| TSB1 | | | | 10 |
| Sphingobium | Sphingobium | Sphingobium | Sphingobium | Sphingobium |
| sp. RAC03 | sp. SYK-6 | sp. TCM1 | sp. TKS | sp. YBL2 |
| Sphingobium | Sphingobium | Sphingobium | Sphingobium | Sphingobium |
| sp. YL23 | sp. YR657 | sp. YR768 | sp. Z007 | ummariense |
| Sphingobium | Sphingobium | Sphingomonadales | Sphingomonas | Sphingomonas |
| xenophagum | yanoikuyae | bacterium EhC05 | adhaesiva | asaccharolytica |
| Sphingomonas | Sphingomonas | Sphingomonas | Sphingomonas | Sphingomonas |
| astaxanthinifaciens | azotifigens | changbaiensis | dokdonensis | echinoides |
| Sphingomonas | Sphingomonas | Sphingomonas | Sphingomonas | Sphingomonas |
| elodea | endophytica | haloaromaticamans | hankookensis | hengshuiensis |
| Sphingomonas | Sphingomonas | Sphingomonas | Sphingomonas | Sphingomonas |
| histidinilytica | indica | jaspsi | koreensis | laterariae |
| Sphingomonas | Sphingomonas | Sphingomonas | Sphingomonas | Sphingomonas |
| mali | melonis | mucosissima | panacis | parapaucimobilis |
| Sphingomonas | Sphingomonas | Sphingomonas | Sphingomonas | Sphingomonas |
| paucimobilis | phyllosphaerae | pituitosa | pruni | rubra |
| Sphingomonas | Sphingomonas | Sphingomonas | Sphingomonas | Sphingomonas |
| sanguinis | sanxanigenens | soli | sp. 35- | sp. 37zxx |
| | | | 24ZXX | |
| Sphingomonas | Sphingomonas | Sphingomonas | Sphingomonas | Sphingomonas |
| sp. Ag1 | sp. Ant H11 | sp. Ant20 | sp. CCH10-B3 | sp. CCH21-G11 |
| Sphingomonas | Sphingomonas | Sphingomonas | Sphingomonas | Sphingomonas |
| sp. CCH5-D11 | sp. CCH9-F2 | sp. CDS-1 | sp. ERG5 | sp. FUKUSWI1S1 |
| Sphingomonas | Sphingomonas | Sphingomonas | Sphingomonas | Sphingomonas |
| sp. G39 | sp. IBVSS1 | sp. IBVSS2 | sp. JJ-A5 | sp. JS21-1 |
| Sphingomonas | Sphingomonas | Sphingomonas | Sphingomonas | Sphingomonas |
| sp. KC8 | sp. Leaf10 | sp. Leaf17 | sp. Leaf198 | sp. Leaf20 |
| Sphingomonas | Sphingomonas | Sphingomonas | Sphingomonas | Sphingomonas |
| sp. Leaf208 | sp. Leaf22 | sp. Leaf226 | sp. Leaf23 | sp. Leaf230 |
| Sphingomonas | Sphingomonas | Sphingomonas | Sphingomonas | Sphingomonas |
| sp. Leaf231 | sp. Leaf242 | sp. Leaf25 | sp. Leaf257 | sp. Leaf28 |
| Sphingomonas | Sphingomonas | Sphingomonas | Sphingomonas | Sphingomonas |
| sp. Leaf30 | sp. Leaf33 | sp. Leaf339 | sp. Leaf34 | sp. Leaf343 |
| Sphingomonas | Sphingomonas | Sphingomonas | Sphingomonas | Sphingomonas |
| sp. Leaf357 | sp. Leaf38 | sp. Leaf412 | sp. Leaf62 | sp. Leaf67 |
| Sphingomonas | Sphingomonas | Sphingomonas | Sphingomonas | Sphingomonas |
| sp. LH128 | sp. LK11 | sp. LM7 | sp. MCT13 | sp. MEA3-1 |
| Sphingomonas | Sphingomonas | Sphingomonas | Sphingomonas | Sphingomonas |
| sp. MM-1 | sp. Mn802worker | sp. Ndbn-20 | sp. NFR04 | sp. NFR15 |
| Sphingomonas | Sphingomonas | Sphingomonas | Sphingomonas | Sphingomonas |
| sp. N1C1 | sp. OK281 | sp. OV641 | sp. PAMC 26605 | sp. PAMC 26617 |
| Sphingomonas | Sphingomonas | Sphingomonas | Sphingomonas | Sphingomonas |
| sp. PAMC 26621 | sp. PR090111-T3T-6A | sp. RIT328 | sp. Root241 | sp. Root710 |
| Sphingomonas | Sphingomonas | Sphingomonas | Sphingomonas | Sphingomonas |
| sp. S5-249 | sp. S6-262 | sp. SKA58 | sp. Sph1(2015) | sp. SRS2 |
| Sphingomonas | Sphingomonas | Sphingomonas | Sphingomonas | Sphingomonas |
| sp. STIS6.2 | sp. TDK1 | sp. TZW2008 | sp. GNC305MFCol5.2 | sp. URHD0057 |
| Sphingomonas | Sphingomonas | Sphingomonas | Sphingomonas | Sphingomonas |
| sp. W16RD | sp. Y57 | sp. YL-JM2C | sp. YR710 | taxi |
| Sphingomonas | Sphingomonas | Sphingomonas- | Sphingopyxis | Sphingopyxis |
| wittichii | yabuuchiae | like bacterium B12 | alaskensis | baekryungensis |
| Sphingopyxis | Sphingopyxis | Sphingopyxis | Sphingopyxis | Sphingopyxis |
| bauzanensis | flava | fribergensis | granuli | indica |
| Sphingopyxis | Sphingopyxis | Sphingopyxis | Sphingopyxis | Sphingopyxis |
| macrogoltabida | sp. 113P3 | sp. C-1 | sp. GW247-27LB | sp. H050 |
| Sphingopyxis | Sphingopyxis | Sphingopyxis | Sphingopyxis | Sphingopyxis |
| sp. H115 | sp. KK2 | sp. LC363 | sp. LC81 | sp. LPB0140 |
| Sphingopyxis | Sphingopyxis | Sphingopyxis | Sphingopyxis | Sphingopyxis |
| sp. MC1 | sp. MWB1 | sp. QXT-31 | sp. Root1497 | sp. YR583 |
| Sphingopyxis | Sphingopyxis | Sphingopyxis | Sphingorhabdus | Sphingorhabdus |
| terrae | ummariensis | witflariensis | flavimaris | marina |
| Sphingorhabdus | Spiribacter | Spiribacter | Spirillospora | Spirochaeta |
| sp. M41 | curvatus | salinus | albida | africana |
| Spirochaeta | Spirochaeta | Spirochaeta | Spiroplasma | Spiroplasma |
| cellobiosiphila | lutea | thermophila | apis | cantharicola |
| Spiroplasma | Spiroplasma | Spiroplasma | Spiroplasma | Spiroplasma |
| chrysopicola | citri | corruscae | culicicola | diminutum |
| Spiroplasma | Spiroplasma | Spiroplasma | Spiroplasma | Spiroplasma |
| eriocheiris | helicoides | kunkelii | litorale | melliferum |
| Spiroplasma | Spiroplasma | Spiroplasma | Spiroplasma | Spiroplasma |
| poulsonii | sabaudiense | syrphidicola | taiwanense | turonicum |
| Spirosoma | Spirosoma | Spirosoma | Spirosoma | Spirosoma |
| aerolatum | endophyticum | luteum | montaniterrae | panaciterrae |
| Spirosoma | Spirosoma | Spirosoma | Spirosoma | Spirulina |
| radiotolerans | rigui | sp. 209 | spitsbergense | major |
| Spirulina | Spizellomyces | Spongiibacter | Spongiibacter | Spongiibacter |
| subsalsa | punctatus | marinus | sp. IMCC21906 | tropicus |
| Spongiibacterium | Sporanaerobacter | Sporanaerobacter | Sporichthya | Sporobacter |
| flavum | acetigenes | sp. PP17-6a | polymorpha | termitidis |
| Sporocytophaga | Sporolactobacillus | Sporolactobacillus | Sporolactobacillus | Sporolactobacillus |
| myxococcoides | inulinus | laevolacticus | nakayamae | terrae |
| Sporolactobacillus | Sporolituus | Sporomusa | Sporomusa | Sporomusa |
| vineae | thermophilus | acidovorans | malonica | ovata |
| Sporomusa | Sporomusa | Sporosarcina | Sporosarcina | Sporosarcina |
| silvacetica | sphaeroides | globispora | koreensis | newyorkensis |
| Sporosarcina | Sporosarcina | Sporosarcina | Sporosarcina | Sporosarcina |
| psychrophila | sp. D27 | sp. EUR3 2.2.2 | sp. HYO08 | sp. P33 |
| Sporosarcina | Sporosarcina | Sporosarcina | Sporothrix | Stackebrandtia |
| sp. P37 | sp. ZBG7A | ureae | schenckii | nassauensis |
| Stanieria | Staphylococcus | Staphylococcus | Staphylococcus | Staphylococcus |
| cyanosphaera | agnetis | argenteus | arlettae | aureus |
| Staphylococcus | Staphylococcus | Staphylococcus | Staphylococcus | Staphylococcus |
| auricularis | capitis | carnosus | chromogenes | cohnii |
| Staphylococcus | Staphylococcus | Staphylococcus | Staphylococcus | Staphylococcus |
| condimenti | delphini | epidermidis | equorum | fleurettii |
| Staphylococcus | Staphylococcus | Staphylococcus | Staphylococcus | Staphylococcus |
| gallinarum | haemolyticus | hominis | hyicus | intermedius |
| Staphylococcus | Staphylococcus | Staphylococcus | Staphylococcus | Staphylococcus |
| lentus | lugdunensis | lutrae | massiliensis | microti |
| Staphylococcus | Staphylococcus | Staphylococcus | Staphylococcus | Staphylococcus |
| muscae | pasteuri | pettenkoferi | phage 2638A | piscifermentans |
| Staphylococcus | Staphylococcus | Staphylococcus | Staphylococcus | Staphylococcus |
| pseudintermedius | saccharolyticus | saprophyticus | schleiferi | schweitzeri |
| Staphylococcus | Staphylococcus | Staphylococcus | Staphylococcus | Staphylococcus |
| sciuri | simiae | simulans | sp. EGD-HP3 | sp. HGB0015 |
| Staphylococcus | Staphylococcus | Staphylococcus | Staphylococcus | Staphylococcus |
| sp. HMSC036D05 | sp. HMSC057G10 | sp. HMSC061G12 | sp. HMSC062A01 | sp. HMSC065D05 |
| Staphylococcus sp. | Staphylococcus | Staphylococcus | Staphylococcus | Staphylococcus |
| HMSC065D11 | sp. HMSC067G10 | sp. HMSC068D07 | sp. HMSC070D05 | sp. HMSC071G07 |
| Staphylococcus | Staphylococcus | Staphylococcus | Staphylococcus | Staphylococcus |
| sp. HMSC072D04 | sp. HMSC072E01 | sp. HMSC14D01 | sp. HMSC62A08 | sp. M0480 |
| Staphylococcus | Staphylococcus | Staphylococcus | Staphylococcus | Staphylococcus |
| sp. NAM3COL9 | sp. OJ82 | stepanovicii | succinus | vitulinus |
| Staphylococcus | Staphylococcus | Staphylothermus | Stappia | Stappia |
| warneri | xylosus | marinus | indica | sp. 2211-S9- |
| | | | | Z10 |
| Stappia | Stappia | Starkeya | Stenotrophomonas | Stenotrophomonas |
| sp. ES.058 | stellulata | novella | acidaminiphila | chelatiphaga |
| Stenotrophomonas | Stenotrophomonas | Stenotrophomonas | Stenotrophomonas | Stenotrophomonas |
| daejeonensis | ginsengisoli | humi | koreensis | maltophilia |
| Stenotrophomonas | Stenotrophomonas | Stenotrophomonas | Stenotrophomonas | Stenotrophomonas |
| nitritireducens | panacihumi | pavanii | pictorum | rhizophila |
| Stenotrophomonas | Stenotrophomonas | Stenotrophomonas | Stenotrophomonas | Stenotrophomonas |
| sp. 92mfcol6.1 | sp. BIIR7 | sp. CC120222-04 | sp. HMSC10F06 | sp. KAs 5-3 |
| Stenotrophomonas | Stenotrophomonas | Stenotrophomonas | Stenotrophomonas | Stenotrophomonas |
| sp. Leaf70 | sp. LM091 | sp. RIT309 | sp. SC-N050 | sp. SKA14 |
| Stenotrophomonas | Stenotrophomonas | Stenotrophomonas | Stenotrophomonas | Stenoxybacter |
| sp. TD3 | sp. WZN-1 | sp. YR347 | terrae | acetivorans |
| Stereum | Steroidobacter | Sterolibacterium | Stigmatella | Stigmatella |
| hirsutum | denitrificans | denitrificans | aurantiaca | erecta |
| Stomatobaculum | Streptacidiphilus | Streptacidiphilus | Streptacidiphilus | Streptacidiphilus |
| longum | albus | anmyonensis | carbonis | jeojiense |
| Streptacidiphilus | Streptacidiphilus | Streptacidiphilus | Streptacidiphilus | Streptacidiphilus |
| jiangxiensis | melanogenes | neutrinimicus | oryzae | rugosus |
| Streptoalloteichus | Streptobacillus | Streptobacillus | Streptobacillus | Streptobacillus |
| hindustanus | felis | hongkongensis | moniliformis | notomytis |
| Streptobacillus | Streptococcus | Streptococcus | Streptococcus | Streptococcus |
| ratti | acidominimus | agalactiae | anginosus | australis |
| Streptococcus | Streptococcus | Streptococcus | Streptococcus | Streptococcus |
| azizii | caballi | canis | castoreus | constellatus |
| Streptococcus | Streptococcus | Streptococcus | Streptococcus | Streptococcus |
| criceti | cristatus | cuniculi | devriesei | didelphis |
| Streptococcus | Streptococcus | Streptococcus | Streptococcus | Streptococcus |
| dysgalactiae | entericus | equi | equinus | ferus |
| Streptococcus | Streptococcus | Streptococcus | Streptococcus | Streptococcus |
| gallolyticus | gordonii | halotolerans | henryi | himalayensis |
| Streptococcus | Streptococcus | Streptococcus | Streptococcus | Streptococcus |
| hyovaginalis | ictaluri | infantarius | infantis | iniae |
| Streptococcus | Streptococcus | Streptococcus | Streptococcus | Streptococcus |
| intermedius | lutetiensis | macacae | macedonicus | marimammalium |
| Streptococcus | Streptococcus | Streptococcus | Streptococcus | Streptococcus |
| marmotae | massiliensis | merionis | minor | mitis |
| Streptococcus | Streptococcus | Streptococcus | Streptococcus | Streptococcus |
| mutans | oralis | orisasini | orisratti | ovis |
| Streptococcus | Streptococcus | Streptococcus | Streptococcus | Streptococcus |
| pantholopis | parasanguinis | parauberis | pasteurianus | peroris |
| Streptococcus | Streptococcus | Streptococcus | Streptococcus | Streptococcus |
| phage 5093 | phage 7201 | phage Dp-1 | phage DT1 | phage O1205 |
| Streptococcus | Streptococcus | Streptococcus | Streptococcus | Streptococcus |
| phage Sfi11 | phage Sfi19 | phage Sfi21 | phage SpSL1 | phage Str-PAP-1 |
| Streptococcus | Streptococcus | Streptococcus | Streptococcus | Streptococcus |
| phage TP-778L | phage TP-J34 | phage YMC-2011 | phocae | plurextorum |
| Streptococcus | Streptococcus | Streptococcus | Streptococcus | Streptococcus |
| pneumoniae | porci | porcinus | pseudopneumoniae | pseudoporcinus |
| Streptococcus | Streptococcus | Streptococcus | Streptococcus | Streptococcus |
| pyogenes | ratti | salivarius | sanguinis | sinensis |
| Streptococcus | Streptococcus | Streptococcus | Streptococcus | Streptococcus |
| sobrinus | sp. 1171_SSPC | sp. 2_1_36FAA | sp. 263_SSPC | sp. 343_SSPC |
| Streptococcus | Streptococcus | Streptococcus | Streptococcus | Streptococcus |
| sp. 400_SSPC | sp. 449_SSPC | sp. 45 | sp. A12 | sp. ACS2 |
| Streptococcus | Streptococcus | Streptococcus | Streptococcus | Streptococcus |
| sp. AS14 | sp. AS20 | sp. C150 | sp. C300 | sp. ‘caviae’ |
| Streptococcus | Streptococcus | Streptococcus | Streptococcus | Streptococcus |
| sp. CCH5-D3 | sp. CCH8-C6 | sp. CCH8-G7 | sp. CCH8-H5 | sp. CCUG 49591 |
| Streptococcus | Streptococcus | Streptococcus | Streptococcus | Streptococcus |
| sp. CM6 | sp. CM7 | sp. DD04 | sp. DD10 | sp. DD12 |
| Streptococcus | Streptococcus | Streptococcus | Streptococcus | Streptococcus |
| sp. DD13 | sp. F0441 | sp. F0442 | sp. FDAARGOS_146 | sp. FDAARGOS_192 |
| Streptococcus | Streptococcus | Streptococcus | Streptococcus | Streptococcus |
| sp. GMD4S | sp. HibF3 | sp. HMSC034A12 | sp. HMSC034B03 | sp. HMSC034B04 |
| Streptococcus | Streptococcus | Streptococcus | Streptococcus | Streptococcus |
| sp. HMSC034B05 | sp. HMSC034E03 | sp. HMSC034E12 | sp. HMSC034F02 | sp. HMSC056C01 |
| Streptococcus | Streptococcus | Streptococcus | Streptococcus | Streptococcus |
| sp. HMSC056D07 | sp. HMSC057E02 | sp. HMSC057G03 | sp. HMSC061D10 | sp. HMSC061E03 |
| Streptococcus | Streptococcus | Streptococcus | Streptococcus | Streptococcus |
| sp. HMSC062D07 | sp. HMSC062H02 | sp. HMSC063B03 | sp. HMSC064D12 | sp. HMSC064H09 |
| Streptococcus | Streptococcus | Streptococcus | Streptococcus | Streptococcus |
| sp. HMSC065C01 | sp. HMSC065E03 | sp. HMSC065H07 | sp. HMSC066E07 | sp. HMSC066F01 |
| Streptococcus | Streptococcus | Streptococcus | Streptococcus | Streptococcus |
| sp. HMSC067A03 | sp. HMSC067H01 | sp. HMSC068F04 | sp. HMSC070B10 | sp. HMSC071D03 |
| Streptococcus | Streptococcus | Streptococcus | Streptococcus | Streptococcus |
| sp. HMSC071H03 | sp. HMSC072C09 | sp. HMSC072D03 | sp. HMSC072D05 | sp. HMSC072D07 |
| Streptococcus | Streptococcus | Streptococcus | Streptococcus | Streptococcus |
| sp. HMSC072G04 | sp. HMSC073A12 | sp. HMSC073D05 | sp. HMSC073F11 | sp. HMSC074B11 |
| Streptococcus | Streptococcus | Streptococcus | Streptococcus | Streptococcus |
| sp. HMSC074F05 | sp. HMSC076C08 | sp. HMSC076C09 | sp. HMSC077D04 | sp. HMSC077F03 |
| Streptococcus | Streptococcus | Streptococcus | Streptococcus | Streptococcus |
| sp. HMSC078D09 | sp. HMSC078H03 | sp. HMSC078H12 | sp. HMSC10A01 | sp. HMSC10E12 |
| Streptococcus | Streptococcus | Streptococcus | Streptococcus | Streptococcus |
| sp. HMSC34B10 | sp. HMSC36C04 | sp. HPH0090 | sp. I-G2 | sp. 1-P16 |
| Streptococcus | Streptococcus | Streptococcus | Streptococcus | Streptococcus |
| sp. KR | sp. M143 | sp. M334 | sp. NLAE-zl-C503 | sp. NZ1587 |
| Streptococcus | Streptococcus | Streptococcus | Streptococcus | Streptococcus |
| sp. OBRC6 | sp. oral | sp. oral | sp. oral | sp. oral |
| | taxon 056 | taxon 058 | taxon 064 | taxon 071 |
| Streptococcus | Streptococcus | Streptococcus | Streptococcus | Streptococcus |
| sp. oral | sp. SK140 | sp. SK643 | sp. SR1 | sp. SR4 |
| taxon 431 | | | | |
| Streptococcus | Streptococcus suis | Streptococcus | Streptococcus | Streptococcus |
| sp. X13SY08 | | thermophilus | thoraltensis | timonensis |
| Streptococcus | Streptococcus | Streptococcus | Streptococcus | Streptococcus |
| uberis | urinalis | varani | vestibularis | virus 9872 |
| Streptomonospora | Streptomyces | Streptomyces | Streptomyces | Streptomyces |
| alba | abyssalis | achromogenes | acidiscabies | afghaniensis |
| Streptomyces | Streptomyces | Streptomyces | Streptomyces | Streptomyces |
| africanus | agglomeratus | aidingensis | albidoflavus | albireticuli |
| Streptomyces | Streptomyces | Streptomyces | Streptomyces | Streptomyces |
| alboflavus | alboniger | alboverticillatus | alboviridis | albulus |
| Streptomyces | Streptomyces | Streptomyces | Streptomyces | Streptomyces |
| albus | alni | ambofaciens | amritsarensis | angustmyceticus |
| Streptomyces | Streptomyces | Streptomyces | Streptomyces | Streptomyces |
| antibioticus | antioxidans | anulatus | atratus | atriruber |
| Streptomyces | Streptomyces | Streptomyces | Streptomyces | Streptomyces |
| atroolivaceus | aurantiacus | auratus | aureocirculatus | aureus |
| Streptomyces | Streptomyces | Streptomyces | Streptomyces | Streptomyces |
| autolyticus | avellaneus | avermitilis | avicenniae | azureus |
| Streptomyces | Streptomyces | Streptomyces | Streptomyces | Streptomyces |
| baarnensis | bicolor | bikiniensis | bingchenggensis | bobili |
| Streptomyces | Streptomyces | Streptomyces | Streptomyces | Streptomyces |
| bottropensis | brasiliensis | bungoensis | caatingaensis | cacaoi |
| Streptomyces | Streptomyces | Streptomyces | Streptomyces | Streptomyces |
| caelestis | caeruleatus | californicus | canus | capuensis |
| Streptomyces | Streptomyces | Streptomyces | Streptomyces | Streptomyces |
| carpinensis | catenulae | cattleya | cellostaticus | celluloflavus |
| Streptomyces | Streptomyces | Streptomyces | Streptomyces | Streptomyces |
| cellulosae | chartreusis | chattanoogensis | cinnamoneus | ciscaucasicus |
| Streptomyces | Streptomyces | Streptomyces | Streptomyces | Streptomyces |
| clavuligerus | coelicoflavus | collinus | corchorusii | curacoi |
| Streptomyces | Streptomyces | Streptomyces | Streptomyces | Streptomyces |
| cvaneogrisens | davawensis | decoyicus | diastatochromogenes | durhamensis |
| Streptomyces | Streptomyces | Streptomyces | Streptomyces | Streptomyces |
| endus | erythrochromogenes | europaeiscabiei | exfoliatus | filamentosus |
| Streptomyces | Streptomyces | Streptomyces | Streptomyces | Streptomyces |
| fimicarius | flavidovirens | flavochromogenes | flavotricini | flocculus |
| Streptomyces | Streptomyces | Streptomyces | Streptomyces | Streptomyces |
| floridae | fradiae | fulvoviolaceus | galbus | gilvigriseus |
| Streptomyces | Streptomyces | Streptomyces | Streptomyces | Streptomyces |
| gilvosporeus | glaucescens | glauciniger | globisporus | graminilatus |
| Streptomyces | Streptomyces | Streptomyces | Streptomyces | Streptomyces |
| griseoaurantiacus | griseochromogenes | griseoflavus | griseofuscus | griseoluteus |
| Streptomyces | Streptomyces | Streptomyces | Streptomyces | Streptomyces |
| griseoplanus | griseorubens | griseoruber | griseonibiginosus | griseus |
| Streptomyces | Streptomyces | Streptomyces | Streptomyces | Streptomyces |
| guanduensis | harbinensis | himastatinicus | hirsutus | hokutonensis |
| Streptomyces | Streptomyces | Streptomyces | Streptomyces | Streptomyces |
| humi | hyaluromycini | hygroscopicus | iakyrus | indicus |
| Streptomyces | Streptomyces | Streptomyces | Streptomyces | Streptomyces |
| ipomoeae | iranensis | jeddahensis | jietaisiensis | kanamyceticus |
| Streptomyces | Streptomyces | Streptomyces | Streptomyces | Streptomyces |
| kanasensis | kasugaensis | katrae | kebangsaanensis | lavendulae |
| Streptomyces | Streptomyces | Streptomyces | Streptomyces | Streptomyces |
| lavenduligriseus | leeuwenhoekii | lincolnensis | lividans | longwoodensis |
| Streptomyces | Streptomyces | Streptomyces | Streptomyces | Streptomyces |
| lushanensis | luteocolor | luteus | lydicus | malaysiense |
| Streptomyces | Streptomyces | Streptomyces | Streptomyces | Streptomyces |
| mangrovisoli | mediolani | megasporus | melanosporofaciens | mirabilis |
| Streptomyces | Streptomyces | Streptomyces | Streptomyces | Streptomyces |
| misionensis | mobaraensis | monomycini | murinus | nanshensis |
| Streptomyces | Streptomyces | Streptomyces | Streptomyces | Streptomyces |
| natalensis | neyagawaensis | niger | niveiscabiei | niveus |
| Streptomyces | Streptomyces | Streptomyces | Streptomyces | Streptomyces |
| nodosus | noursei | novaecaesareae | oceani | ochraceiscleroticus |
| Streptomyces | Streptomyces | Streptomyces | Streptomyces | Streptomyces |
| olindensis | olivaceus | olivochromogenes | ossamyceticus | pactum |
| Streptomyces | Streptomyces | Streptomyces | Streptomyces | Streptomyces |
| parvulus | pathocidini | paucisporeus | peucetius | phaeochromogenes |
| Streptomyces | Streptomyces | Streptomyces | Streptomyces | Streptomyces |
| phaeoluteigriseus | phaeopurpureus | phage Amela | pharetrae | pini |
| Streptomyces | Streptomyces | Streptomyces | Streptomyces | Streptomyces |
| platensis | prasinopilosus | prasinus | pristinaespiralis | prunicolor |
| Streptomyces | Streptomyces | Streptomyces | Streptomyces | Streptomyces |
| puniciscabiei | purpureus | purpurogeneiscleroticus | qinglanensis | radiopugnans |
| Streptomyces | Streptomyces | Streptomyces | Streptomyces | Streptomyces |
| rangoonensis | recifensis | regalis | resistomycificus | reticuli |
| Streptomyces | Streptomyces | Streptomyces | Streptomyces | Streptomyces |
| rhizosphaericus | rimosus | rochei | roseochromogenus | roseoverticillatus |
| Streptomyces | Streptomyces | Streptomyces | Streptomyces | Streptomyces |
| rubellomurinus | ruber | nibidus | sampsonii | scabiei |
| Streptomyces | Streptomyces | Streptomyces | Streptomyces | Streptomyces |
| scabrisporus | sclerotialus | scopuliridis | seoulensis | silaceus |
| Streptomyces | Streptomyces | Streptomyces | Streptomyces | Streptomyces |
| silvensis | somaliensis | sp. 1222.5 | sp. 13- | sp. 142MFCol3.1 |
| | | | 12-16 | |
| Streptomyces | Streptomyces | Streptomyces | Streptomyces | Streptomyces |
| sp. 150FB | sp. 2131.1 | sp. 2224.1 | sp. 2231.1 | sp. 2R |
| Streptomyces | Streptomyces | Streptomyces | Streptomyces | Streptomyces |
| sp. 303MFCol5.2 | sp. 3124.6 | sp. 3211 | sp. 3213 | sp. 351MFTsu5.1 |
| Streptomyces | Streptomyces | Streptomyces | Streptomyces | Streptomyces |
| sp. 769 | sp. AA0539 | sp. AA4 | sp. AcH | sp. ACT-1 |
| | | | 505 | |
| Streptomyces | Streptomyces | Streptomyces | Streptomyces | Streptomyces |
| sp. Ag109_O5-10 | sp. Amel2xC10 | sp. Amel2xE9 | sp. AS58 | sp. ATexAB-D23 |
| Streptomyces | Streptomyces | Streptomyces | Streptomyces | Streptomyces |
| sp. AVP053U2 | sp. AW19M42 | sp. B9173 | sp. BoleA5 | sp. BpilaLS-43 |
| Streptomyces | Streptomyces | Streptomyces | Streptomyces | Streptomyces |
| sp. CB00072 | sp. CB00316 | sp. CB00455 | sp. CB01249 | sp. CB01580 |
| Streptomyces | Streptomyces | Streptomyces | Streptomyces | Streptomyces |
| sp. CB01883 | sp. CB02009 | sp. CB02056 | sp. CB02058 | sp. CB02115 |
| Streptomyces | Streptomyces | Streptomyces | Streptomyces | Streptomyces |
| sp. CB02130 | sp. CB02261 | sp. CB02366 | sp. CB02400 | sp. CB02414 |
| Streptomyces | Streptomyces | Streptomyces | Streptomyces | Streptomyces |
| sp. CB02460 | sp. CB02488 | sp. CB02923 | sp. CB03234 | sp. CB03238 |
| Streptomyces | Streptomyces | Streptomyces | Streptomyces | Streptomyces |
| sp. CB03578 | sp. CB03911 | sp. CC53 | sp. CC71 | sp. CC77 |
| Streptomyces | Streptomyces | Streptomyces | Streptomyces | Streptomyces |
| sp. CcalMP-8W | sp. CCM_MD2014 | sp. CdTB01 | sp. cf124 | sp. cf136 |
| Streptomyces | Streptomyces | Streptomyces | Streptomyces | Streptomyces |
| sp. CFMR 7 | sp. CNB091 | sp. CNH099 | sp. CNH189 | sp. CNH287 |
| Streptomyces | Streptomyces | Streptomyces | Streptomyces | Streptomyces |
| sp. CNQ329 | sp. CNQ431 | sp. CNQ- | sp. CNQ- | sp. CNQ766 |
| | | 509 | 525 | |
| Streptomyces | Streptomyces | Streptomyces | Streptomyces | Streptomyces |
| sp. CNQ865 | sp. CNR698 | sp. CNS606 | sp. CNS615 | sp. CNS654 |
| Streptomyces | Streptomyces | Streptomyces | Streptomyces | Streptomyces |
| sp. CNT302 | sp. CNT318 | sp. CNT360 | sp. CNT371 | sp. CNT372 |
| Streptomyces | Streptomyces | Streptomyces | Streptomyces | Streptomyces |
| sp. CNY243 | sp. CS057 | sp. CS113 | sp. CS227 | sp. CT34 |
| Streptomyces | Streptomyces | Streptomyces | Streptomyces | Streptomyces |
| sp. D1166 | sp. DSM | sp. DvalAA-14 | sp. DvalAA-19 | sp. DvalAA-43 |
| | 15324 | | | |
| Streptomyces | Streptomyces | Streptomyces | Streptomyces | Streptomyces |
| sp. e14 | sp. EN16 | sp. EN23 | sp. ERV7 | sp. F1 |
| Streptomyces | Streptomyces | Streptomyces | Streptomyces | Streptomyces |
| sp. F-3 | sp. F-7 | sp. FBKL.4005 | sp. fd1-xmd | sp. FR- |
| | | | | 008 |
| Streptomyces | Streptomyces | Streptomyces | Streptomyces | Streptomyces |
| sp. FxanaA7 | sp. FxanaC1 | sp. FxanaD5 | sp. FXJ1.172 | sp. FXJ7.023 |
| Streptomyces | Streptomyces | Streptomyces | Streptomyces | Streptomyces |
| sp. GKU 895 | sp. H021 | sp. H036 | sp. HGB0020 | sp. H-KF8 |
| Streptomyces | Streptomyces | Streptomyces | Streptomyces | Streptomyces |
| sp. HmicA12 | sp. HNS054 | sp. HPH0547 | sp. IB2014 011-1 | sp. IGB124 |
| Streptomyces | Streptomyces | Streptomyces | Streptomyces | Streptomyces |
| sp. IMTB 2501 | sp. JHA19 | sp. JHA26 | sp. JS01 | sp. KE1 |
| Streptomyces | Streptomyces | Streptomyces | Streptomyces | Streptomyces |
| sp. KS_5 | sp. LaPpAH-108 | sp. LaPpAH-202 | sp. LcepLS | sp. LUP30 |
| Streptomyces | Streptomyces | Streptomyces | Streptomyces | Streptomyces |
| sp. LUP47B | sp. M10 | sp. M1013 | sp. M41(2017) | sp. MBT28 |
| Streptomyces | Streptomyces | Streptomyces | Streptomyces | Streptomyces |
| sp. MBT76 | sp. Mg1 | sp. MJM1172 | sp. MJM8645 | sp. MMG1064 |
| Streptomyces | Streptomyces | Streptomyces | Streptomyces | Streptomyces |
| sp. MMG1121 | sp. MMG1522 | sp. MMG1533 | sp. MnatMP-M17 | sp. MnatMP-M27 |
| Streptomyces | Streptomyces | Streptomyces | Streptomyces | Streptomyces |
| sp. MnatMP-M77 | sp. MOE7 | sp. MP131-18 | sp. MspMP-M5 | sp. MUSC 1 |
| Streptomyces | Streptomyces | Streptomyces | Streptomyces | Streptomyces |
| sp. MUSC 125 | sp. MUSC 14 | sp. MUSC 93 | sp. NBRC 109436 | sp. NBRC 109706 |
| Streptomyces | Streptomyces | Streptomyces | Streptomyces | Streptomyces |
| sp. NBRC110027 | sp. NBRC 110028 | sp. NBRC 110030 | sp. NBRC 110035 | sp. NBRC 110465 |
| Streptomyces | Streptomyces | Streptomyces | Streptomyces | Streptomyces |
| sp. NBRC 110468 | sp. NBRC 110611 | sp. NBS 14/10 | sp. Ncost-T10-10d | sp. Ncost-T6T-1 |
| Streptomyces | Streptomyces | Streptomyces | Streptomyces | Streptomyces |
| sp. NRRL | sp. NRRL | sp. NRRL | sp. NRRL | sp. NRRL |
| B-11253 | B-1140 | B-1347 | B-1381 | B-1568 |
| Streptomyces | Streptomyces | Streptomyces | Streptomyces | Streptomyces |
| sp. NRRL | sp. NRRL | sp. NRRL | sp. NRRL | sp. NRRL |
| B-24051 | B-24085 | B-24484 | B-24572 | B-24720 |
| Streptomyces | Streptomyces | Streptomyces | Streptomyces | Streptomyces |
| sp. NRRL | sp. NRRL | sp. NRRL | sp. NRRL | sp. NRRL |
| B-24891 | B-3229 | B-3253 | B-3648 | B-5680 |
| Streptomyces | Streptomyces | Streptomyces | Streptomyces | Streptomyces |
| sp. NRRL | sp. NRRL | sp. NRRL | sp. NRRL | sp. NRRL |
| F-2202 | F-2295 | F-2305 | F-2580 | F-2664 |
| Streptomyces | Streptomyces | Streptomyces | Streptomyces | Streptomyces |
| sp. NRRL | sp. NRRL | sp. NRRL | sp. NRRL | sp. NRRL |
| F-2747 | F-2799 | F-2890 | F-3213 | F-3218 |
| Streptomyces | Streptomyces | Streptomyces | Streptomyces | Streptomyces |
| sp. NRRL | sp. NRRL | sp. NRRL | sp. NRRL | sp. NRRL |
| F-4335 | F-4474 | F-4489 | F-4707 | F-4711 |
| Streptomyces | Streptomyces | Streptomyces | Streptomyces | Streptomyces |
| sp. NRRL | sp. NRRL | sp. NRRL | sp. NRRL | sp. NRRL |
| F-5053 | F-5065 | F-5122 | F-5123 | F-5126 |
| Streptomyces | Streptomyces | Streptomyces | Streptomyces | Streptomyces |
| sp. NRRL | sp. NRRL | sp. NRRL | sp. NRRL | sp. NRRL |
| F-5135 | F-5193 | F-525 | F-5527 | F-5555 |
| Streptomyces | Streptomyces | Streptomyces | Streptomyces | Streptomyces |
| sp. NRRL | sp. NRRL | sp. NRRL | sp. NRRL | sp. NRRL |
| F-5630 | F-5639 | F-5650 | F-5681 | F-5702 |
| Streptomyces | Streptomyces | Streptomyces | Streptomyces | Streptomyces |
| sp. NRRL | sp. NRRL | sp. NRRL | sp. NRRL | sp. NRRL |
| F-5727 | F-5755 | F-5917 | F-6131 | F-6491 |
| Streptomyces | Streptomyces | Streptomyces | Streptomyces | Streptomyces |
| sp. NRRL | sp. NRRL | sp. NRRL | sp. NRRL | sp. NRRL |
| F-6628 | F-6674 | F-6676 | F-6677 | F-7442 |
| Streptomyces | Streptomyces | Streptomyces | Streptomyces | Streptomyces |
| sp. NRRL | sp. NRRL | sp. NRRL | sp. NRRL | sp. NRRL |
| S-1022 | S-118 | S-1448 | S-146 | S-15 |
| Streptomyces | Streptomyces | Streptomyces | Streptomyces | Streptomyces |
| sp. NRRL | sp. NRRL | sp. NRRL | sp. NRRL | sp. NRRL |
| S-1521 | S-1813 | S-1824 | S-1831 | S-1868 |
| Streptomyces | Streptomyces | Streptomyces | Streptomyces | Streptomyces |
| sp. NRRL | sp. NRRL | sp. NRRL | sp. NRRL | sp. NRRL |
| S-1896 | S-237 | S-241 | S-244 | S-31 |
| Streptomyces | Streptomyces | Streptomyces | Streptomyces | Streptomyces |
| sp. NRRL | sp. NRRL | sp. NRRLS-340 | sp. NRRL S-350 | sp. NRRL S-37 |
| S-325 | S-337 | | | |
| Streptomyces | Streptomyces | Streptomyces | Streptomyces | Streptomyces |
| sp. NRRL S-378 | sp. NRRL | sp. NRRL S-4 | sp. NRRL S-455 | sp. NRRL S-475 |
| | S-384 | | | |
| Streptomyces | Streptomyces | Streptomyces | Streptomyces | Streptomyces |
| sp. NRRL S-495 | sp. NRRL S-515 | sp. NRRL S-646 | sp. NRRL S-813 | sp. NRRL S-87 |
| Streptomyces | Streptomyces | Streptomyces | Streptomyces | Streptomyces |
| sp. NRR S-920 | sp. NRRL WC-3540 | sp. NRRL WC-3549 | sp. NRRL WC-3604 | sp. NRRL WC-3618 |
| Streptomyces | Streptomyces | Streptomyces | Streptomyces | Streptomyces |
| sp. NRRL WC-3626 | sp. NRRL WC-3701 | sp. NRRL WC-3719 | sp. NRRL WC-3723 | sp. NRRL WC-3742 |
| Streptomyces | Streptomyces | Streptomyces | Streptomyces | Streptomyces |
| sp. NRRL WC-3744 | sp. NRRL WC-3795 | sp. NTK 937 | sp. OspMP-M43 | sp. PAMC 26508 |
| Streptomyces | Streptomyces | Streptomyces | Streptomyces | Streptomyces |
| sp. PAN FS17 | sp. PCS3-D2 | sp. PRh5 | sp. PsTaAH-124 | sp. PTY087I2 |
| Streptomyces | Streptomyces | Streptomyces | Streptomyces | Streptomyces |
| sp. Root1310 | sp. Root264 | sp. Root369 | sp. Root431 | sp. RTd22 |
| Streptomyces | Streptomyces | Streptomyces | Streptomyces | Streptomyces |
| sp. RV15 | sp. S10(2016) | sp. S4 | sp. S8 | sp. SAT1 |
| Streptomyces | Streptomyces | Streptomyces | Streptomyces | Streptomyces |
| sp. SBT349 | sp. ScaeMP-6W | sp. ScaeMP-e10 | sp. ScaeMP-e83 | sp. SCS1O 03032 |
| Streptomyces | Streptomyces | Streptomyces | Streptomyces | Streptomyces |
| sp. SolWspMP-5a-2 | sp. SolWspMP-sol2th | sp. SolWspMP-sol7th | sp. SPB074 | sp. SPB78 |
| Streptomyces | Streptomyces | Streptomyces | Streptomyces | Streptomyces |
| sp. SPMA113 | sp. SS07 | sp. TAA040 | sp. TAA204 | sp. TAA486 |
| Streptomyces | Streptomyces | Streptomyces | Streptomyces | Streptomyces |
| sp. TLI_053 | sp. TLI_105 | sp. TOR3209 | sp. TP-A0356 | sp. TP-A0874 |
| Streptomyces | Streptomyces | Streptomyces | Streptomyces | Streptomyces |
| sp. TSRI0107 | sp. TSRI0261 | sp. TSRI0281 | sp. Tu 6176 | sp. Tu6071 |
| Streptomyces | Streptomyces | Streptomyces | Streptomyces | Streptomyces |
| sp. Tue 6075 | sp. UNC401CLCol | sp. URHA0041 | sp. W007 | sp. WAC04657 |
| Streptomyces | Streptomyces | Streptomyces | Streptomyces | Streptomyces |
| sp. Wb2n-11 | sp. WM4235 | sp. WM6349 | sp. WM6368 | sp. WM6372 |
| Streptomyces | Streptomyces | Streptomyces | Streptomyces | Streptomyces |
| sp. WM6378 | sp. WM6386 | sp. WM6391 | sp. WMMB 322 | sp. WMMB 714 |
| Streptomyces | Streptomyces | Streptomyces | Streptomyces | Streptomyces |
| sp. XY006 | sp. XY152 | sp. XY332 | sp. XY37 | sp. XY413 |
| Streptomyces | Streptomyces | Streptomyces | Streptomyces | Streptomyces |
| sp. XY431 | sp. XY511 | sp. XY533 | sp. XY58 | sp. XY66 |
| Streptomyces | Streptomyces | Streptomyces | Streptomyces | Streptomyces |
| sp. yr375 | sparsogenes | specialis | sporocinereus | stelliscabiei |
| Streptomyces | Streptomyces | Streptomyces | Streptomyces | Streptomyces |
| subrutilus | sulphureus | sviceus | swartbergensis | thermoautotrophicus |
| Streptomyces | Streptomyces | Streptomyces | Streptomyces | Streptomyces |
| thermolilacinus | thermovulgaris | toralosus | toyocaensis | tsukubensis |
| Streptomyces | Streptomyces | Streptomyces | Streptomyces | Streptomyces |
| turhidiscabies | uncialis | varsoviensis | venezuelae | vietnamensis |
| Streptomyces | Streptomyces | Streptomyces | Streptomyces | Streptomyces |
| vinaceus | violaceoruber | violaceorubidus | violaceusniger | violens |
| Streptomyces | Streptomyces | Streptomyces | Streptomyces | Streptomyces |
| virginiae | viridifaciens | viridochromogenes | viridosporus | vitaminophilus |
| Streptomyces | Streptomyces | Streptomyces | Streptomyces | Streptomyces |
| wuyuanensis | xanthophaeus | xiamenensis | xiaopingdaonensis | xylophagus |
| Streptomyces | Streptomyces | Streptomyces | Streptomyces | Streptomyces |
| yangpuensis | yeochonensis | yerevanensis | yokosukanensis | yunnanensis |
| Streptomyces | Streptomycetaceae | Streptosporangium | Streptosporangium | Streptosporangium |
| zinciresistens | bacterium MP113-05 | amethystogenes | canum | roseum |
| Streptosporangium | Streptosporangium | Subdoligranulum | Subdoligranulum | Succinatimonas |
| sp. M26 | subroseum | sp. 4_3_54A2FAA | variabile | hippei |
| Succiniclasticum | Succinimonas | Succinispira | Succinivibrio | Succinivibrionaceae |
| ruminis | amylolytica | mobilis | dextrinosolvens | bacterium WG-1 |
| Sugiyamaella | Sulfitobacter | Sulfitobacter | Sulfitobacter | Sulfitobacter |
| lignohabitans | brevis | delicatus | donghicola | dubius |
| Sulfitobacter | Sulfitobacter | Sulfitobacter | Sulfitobacter | Sulfitobacter |
| geojensis | guttiformis | litoralis | marinus | mediterraneus |
| Sulfitobacter | Sulfitobacter | Sulfitobacter | Sulfitobacter | Sulfitobacter |
| noctilucae | noctilucicola | pontiacus | pseudonitzschiae | sp. 20 GPM-1509m |
| Sulfitobacter | Sulfitobacter | Sulfitobacter | Sulfitobacter | Sulfitobacter |
| sp. AM1-D1 | sp. CB2047 | sp. EE-36 | sp. EhC04 | sp. HI0054 |
| Sulfitobacter | Sulfobacillus | Sulfolobus | Sulfolobus | Sulfolobus |
| sp. NAS-14.1 | thermosulfidooxidans | metallicus | solfataricus | sp. A20 |
| Sulfuricella | Sulfuricella | Sulfuricurvum | Sulfuriferula | Sulfurihydrogenibium |
| denitrificans | sp. T08 | kujiense | sp. AH1 | azorense |
| Sulfurihydrogenibium | Sulfurihydrogenibium | Sulfurihydrogenibium | Sulfurimonas | Sulfurimonas |
| sp. YO3AOP1 | subterraneum | yellowstonense | autotrophica | denitrificans |
| Sulfurimonas | Sulfurimonas | Sulfuritalea | Sulfurivirga | Sulfurospirillum |
| gotlandica | hongkongensis | hydrogenivorans | caldicuralii | arcachonense |
| Sulfurospirillum | Sulfurospirillum | Sulfurospirillum | Sulfurospirillum | Sulfurospirillum |
| arsenophilum | barnesii | cavolei | deleyianum | halorespirans |
| Sulfurospirillum | Sulfurospirillum | Sulfurovum | Sulfurovum | Sulfurovum |
| multivorans | sp. UCH001 | lithotrophicum | riftiae | sp. AR |
| Sulfiirovum | Sunxiuqinia | Sunxiuqinia | Sutterella | Sutterella |
| sp. NBC37-1 | dokdonensis | elliptica | parvirubra | sp. KLE1602 |
| Sutterella | Suttonella | Symbiobacterium | Synechococcus | Synechococcus |
| wadsworthensis | ornithocola | thermophilum | sp. 1G10 | sp. 8F6 |
| Synechococcus | Synechococcus | Synechococcus | Synechococcus | Synechococcus |
| sp. BL107 | sp. BO 8801 | sp. CB0101 | sp. CB0205 | sp. CC9311 |
| Synechococcus | Synechococcus | Synechococcus | Synechococcus | Synechococcus |
| sp. CC9605 | sp. CC9616 | sp. CC9902 | sp. GFB01 | sp. JA-2-3B′a(2-13) |
| Synechococcus | Synechococcus | Synechococcus | Synechococcus | Synechococcus |
| sp. JA-3-3Ab | sp. KORDI-100 | sp. KORDI-49 | sp. KORDI-52 | sp. LL |
| Synechococcus | Synechococcus | Synechococcus | Synechococcus | Synechococcus |
| sp. MIT S9504 | sp. MIT S9508 | sp. MIT S9509 | sp. MW101C3 | sp. NKBG15041C |
| Synechococcus | Synechococcus | Synechococcus | Synechococcus | Synechococcus |
| sp. PCC 6312 | sp. PCC 7003 | sp. PCC 7117 | sp. PCC 7335 | sp. PCC 7336 |
| Synechococcus | Synechococcus | Synechococcus | Synechococcus | Synechococcus |
| sp. PCC 7502 | sp. PCC 8807 | sp. RCC307 | sp. RS9916 | sp. RS9917 |
| Synechococcus | Synechococcus | Synechococcus | Synechococcus | Synechococcus |
| sp. SynAce01 | sp. WH 5701 | sp. WH 7803 | sp. WH 7805 | sp. WH 8016 |
| Synechococcus | Synechococcus | Synechococcus | Synechococcus | Synechocystis |
| sp. WH 8020 | sp. WH 8102 | sp. WH 8103 | sp. WH 8109 | sp. PCC 6714 |
| Synechocystis | Synechocystis | Synergistes | Synergistes | Syntrophaceticus |
| sp. PCC 6803 | sp. PCC 7509 | jonesii | sp. 3_1_syn1 | schinkii |
| Syntrophobacter | Syntrophobotulus | Syntrophomonas | Syntrophomonas | Syntrophomonas |
| fumaroxidans | glycolicus | palmitatica | wolfei | zehnderi |
| Syntrophorhabdus | Syntrophothermus | Syntrophus | Syntrophus | Talaromyces |
| aromaticivorans | lipocalidus | aciditrophicus | gentianae | atroroseus |
| Talaromyces | Talaromyces | Tamlana | Tamlana | Tamlana |
| marneffei | stipitatus | agarivorans | nanhaiensis | sedimentorum |
| Tamlana | Tangfeifania | Tannerella | Tannerella | Tannerella |
| sp. s12 | diversioriginum | forsythia | sp. 6_1_58FAA_CT1 | sp. oral |
| | | | | taxon HOT-286 |
| Tanticharoenia | Tardiphaga | Tardiphaga | Tardiphaga | Tateyamaria |
| sakaeratensis | robiniae | sp. OK245 | sp. OK246 | omphalii |
| Tateyamaria | Tatumella | Tatumella | Tatumella | Tatumella sp. UCD- |
| sp. ANG-S1 | citrea | morbirosei | saanichensis | D suzukii |
| Taylorella | Taylorella | Tenacibaculum | Tenacibaculum | Tenacibaculum |
| asinigenitalis | equigenitalis | dicentrarchi | holothuriorum | jejuense |
| Tenacibaculum | Tenacibaculum | Tenacibaculum | Tenacibaculum | Tenacibaculum |
| maritimum | mesophilum | ovolyticum | soleae | sp. 47A_GOM-205m |
| Tenacibaculum | Tenacibaculum | Tenacibaculum | Tenacibaculum | Tenuibacillus |
| sp. HZ1 | sp. LPB0136 | sp. MAR_2009_124 | sp. MAR_2010_89 | multivorans |
| Tepidanaerobacter | Tepidanaerobacter | Tepidibacillus | Tepidibacillus | Tepidibacter |
| acetatoxydans | syntrophicus | decaturensis | sp. HK-1 | formicigenes |
| Tepidibacter | Tepidicaulis | Tepidimicrobium | Tepidimonas | Tepidimonas |
| thalassicus | marinas | xylanilyticum | fonticaldi | taiwanensis |
| Tepidiphilus | Tepidiphilus | Terasakiella | Terasakiella | Terasakiispira |
| margaritifer | thermophilus | pusilia | sp. PR1 | papahanaumokuakeensis |
| Teredinibacter | Teredinibacter | Teredinibacter | Terrabacter | Terrabacter |
| sp. 1162T.S.0a.05 | sp. 991H.S.0a.06 | turnerae | sp. 28 | sp. Root181 |
| Terrabacter | Terracidiphilus | Terracoccus | Terribacillus | Terribacillus |
| sp. Root85 | gabretensis | sp. 273MFTsu3.1 | aidingensis | halophilus |
| Terribacillus | Terribacillus | Terriglobus | Terriglobus | Terriglobus |
| saccharophilus | sp. 7520-G | roseus | saanensis | sp. TAA 43 |
| Terrimicrobium | Terrimonas | Terrisporobacter | Terrisporobacter | Tersicoccus |
| sacchariphilum | ferruginea | glycolicus | othiniensis | phoenicis |
| Tersicoccus | Tessaracoccus | Tessaracoccus | Tessaracoccus | Tessaracoccus |
| sp. Bi-70 | bendigoensis | flavescens | flavus | massiliensis |
| Tessaracoccus | Tessaracoccus | Tessaracoccus | Tessaracoccus | Tetragenococcus |
| oleiagri | sp. NSG39 | sp. T2.5-30 | sp. ZS01 | halophilus |
| Tetragenococcus | Tetragenococcus | Tetrahymena | Tetrapisispora | Tetrapisispora |
| muriaticus | solitarius | thermophila | blattae | phaffii |
| Tetrasphaera | Tetrasphaera | Tetrasphaera | Tetrasphaera | Tetrasphaera |
| australiensis | elongata | japonica | jenkinsii | phage TJE1 |
| Tetrasphaera | Thalassiosira | Thalassobacillus | Thalassobacillus | Thalassobacillus |
| sp. Soil756 | pseudonana | cyri | devorans | sp. C254 |
| Thalassobacillus | Thalassobacter | Thalassobaculum | Thalassobaculum | Thalassobium |
| sp. TM-1 | stenotrophicus | litoreum | salexigens | sp. R2A62 |
| Thalassobius | Thalassobius | Thalassobius | Thalassobius | Thalassolituus |
| aestuarii | gelatinovorus | maritimus | mediterraneus | oleivorans |
| Thalassomonas | Thalassomonas | Thalassospira | Thalassospira | Thalassospira |
| actiniarum | viridans | alkalitolerans | australica | lucentensis |
| Thalassospira | Thalassospira | Thalassospira | Thalassospira | Thalassospira |
| mesophila | permensis | profundimaris | sp. HJ | sp. MCCC 1A01148 |
| Thalassospira | Thalassospira | Thalassospira | Thalassospira | Thalassospira |
| sp. MCCC 1A01428 | sp. MCCC 1A02491 | sp. MCCC 1A03138 | sp. TSL5-1 | tepidiphila |
| Thalassospira | Thalassotalea | Thalassotalea | Thalassotalea | Thalassotalea |
| xiamenensis | agarivorans | crassostreae | sp. ND16A | sp. PP2-459 |
| Thauera | Thauera | Thauera | Thauera | Thauera |
| aminoaromatica | butanivorans | chlorobenzoica | humireducens | linaloolentis |
| Thauera | Thauera | Thauera | Thauera | Thauera |
| phenylacetica | sp. 27 | sp. 28 | sp. 63 | sp. KNDSS-Mac4 |
| Thauera | Thauera sp. SWB20 | Thauera | Thauera | Thaumarchaeota |
| sp. MZ1T | | sp. ZV-l-C | terpenica | archaeon N4 |
| Thaumasiovibrio | Thaumasiovibrio | Thecamonas | Theileria | Theileria |
| occultus | subtropicus | trahens | annulata | equi |
| Theileria | Theileria | Thermacetogenium | Thermaerobacter | Thermaerobacter |
| orientalis | parva | phaeum | marianensis | subterraneus |
| Thermanaeromonas | Thermanaerothrix | Thermanaerovibrio | Thermanaerovibrio | Thermicanus |
| toyohensis | daxensis | acidaminovorans | velox | aegyptius |
| Thermincola | Thermincola | Thermithiobacillus | Thermoactinomyces | Thermoactinomyces |
| ferriacetica | potens | tepidarius | daqus | sp. CDF |
| Thermoactinomyces | Thermoactinomyces | Thermoactinomyces | Thermoactinomyces | Thermoactinospora |
| sp. DSM 45891 | sp. DSM 45892 | sp. Gus2-1 | vulgaris | rubra |
| Thermoanaerobacter | Thermoanaerobacter | Thermoanaerobacter | Thermoanaerobacter | Thermoanaerobacter |
| cellulolyticiis | ethanolicus | italicus | kivui | mathranii |
| Thermoanaerobacter | Thermoanaerobacter | Thermoanaerobacter | Thermoanaerobacter | Thermoanaerobacter |
| pseudethanolicus | siderophilus | sp. YS13 | thermocopriae | thermohydrosulfuricus |
| Thermoanaerobacter | Thermoanaerobacter | Thermoanaerobacterium | Thermoanaerobacterium | Thermoanaerobacterium |
| uzonensis | wiegelii | aotearoense | saccharolyticum | sp. PSU-2 |
| Thermoanaerobacterium | Thermoanaerobacterium | Thermoanaerobaculum | Thermobacillus | Thermobaculum |
| thermosaccharolyticum | xylanolyticum | aquaticum | composti | terrenum |
| Thermobiflda | Thermobifida | Thermobifida | Thermobispora | Thermobrachium |
| cellulosilytica | fusca | halotolerans | bispora | celere |
| Thermococcus | Thermococcus | Thermococcus | Thermococcus | Thermococcus |
| barophilus | barossii | celer | celericrescens | chitonophagus |
| Thermococcus | Thermococcus | Thermococcus | Thermococcus | Thermococcus |
| cleftensis | eurythermalis | gammatolerans | guaymasensis | kodakarensis |
| Thermococcus | Thermococcus | Thermococcus | Thermococcus | Thermococcus |
| litoralis | nautili | onnurineus | pacificus | paralvinellae |
| Thermococcus | Thermococcus | Thermococcus | Thermococcus | Thermococcus |
| peptonophilus | piezophilus | profundus | sibiricus | siculi |
| Thermococcus | Thermococcus | Thermococcus | Thermococcus | Thermococcus |
| sp. 4557 | sp. 5-4 | sp. AM4 | sp. P6 | sp. PK |
| Thermococcus | Thermococcus | Thermocrinis | Thermocrinis | Thermocrinis |
| thioreducens | zilligii | albus | minervae | ruber |
| Thermocrinis | Thermocrispum | Thermocrispum | Thermodesulfatator | Thermodesulfatator |
| sp. GBS | agreste | municipale | atlanticus | autotrophicus |
| Thermodesulfatator | Thermodesulfobacterium | Thermodesulfobacterium | Thermodesulfobacterium | Thermodesulfobacterium |
| indicus | commune | geofontis | hveragerdense | hydrogeniphilum |
| Thermodesulfobacterium | Thermodesulfobium | Thermodesulforhabdus | Thermodesulfovibrio | Thermodesulfovibrio |
| thermophilum | narugense | norvegica | aggregans | islandicus |
| Thermodesulfovibrio | Thermodesulfovibrio | Thermodesulfovibrio | Thermofilum | Thermoflavimicrobium |
| sp. N1 | thiophilus | yellowstonii | pendens | dichotomicum |
| Thermoflexibacter | Thermoflexus | Thermogemmatispora | Thermogemmatispora | Thermogladius |
| ruber | hugenholtzii | carboxidivorans | onikobensis | calderae |
| Thermogutta | Thermoleophilum | Thermomicrobium | Thermomonas | Thermomonas |
| terrifontis | album | roseum | fusca | hydrothermalis |
| Thermomonospora | Thermomonospora | Thermonema | Thermopetrobacter | Thermophagus |
| chromogena | curvata | rossianum | sp. TC1 | xiamenensis |
| Thermoplasma | Thermoplasmatales | Thermoproteus | Thermoproteus | Thermorudis |
| volcanium | archaeon BRNA1 | tenax | uzoniensis | peleae |
| Thermosediminibacter | Thermosinus | Thermosipho | Thermosipho | Thermosipho |
| oceani | carboxydivorans | affectus | africanus | atlanticus |
| Thermosipho | Thermosipho | Thermosipho | Thermosulfidibacter | Thermosulfurimonas |
| melanesiensis | sp. 1070 | sp. 1223 | takaii | dismutans |
| Thermosynechococcus | Thermosynechococcus | Thermosyntropha | Thermotalea | Thermothelomyces |
| elongatus | sp. NK55a | lipolytica | metallivorans | thermophila |
| Thermotoga | Thermotoga | Thermotoga | Thermotoga | Thermotoga |
| caldifontis | maritima | neapolitana | profunda | sp. Mc24 |
| Thermotoga | Thermovenabulum | Thermovibrio | Thermovirga | Thermus |
| sp. RQ7 | gondwanense | ammonificans | lienii | amyloliquefaciens |
| Thermus | Thermus | Thermus | Thermus | Thermus |
| antranikianii | aquaticus | arciformis | brockianus | caliditerrae |
| Thermus | Thermus | Thermus | Thermus | Thermus |
| filiformis | igniterrae | islandicus | oshimai | parvatiensis |
| Thermus | Thermus | Thermus | Thermus | Thermus |
| phage TMA | scotoductus | sp. 2.9 | sp. CCB_US3_UF1 | sp. NMX2.A1 |
| Thermus | Thermus | Thielavia | Thioalkalimicrobium | Thioalkalimicrobium |
| tengchongensis | thermophilus | terrestris | aerophilum | cyclicum |
| Thioalkalimicrobium | Thioalkalimicrobium | Thioalkalivibrio | Thioalkalivibrio | Thioalkalivibrio |
| microaerophilum | sp. ALE5 | denitrificans | halophilus | nitratireducens |
| Thioalkalivibrio | Thioalkalivibrio | Thioalkalivibrio | Thioalkalivibrio | Thioalkalivibrio |
| paradoxus | sp. AKL10 | sp. AKL11 | sp. AKL12 | sp. AKL17 |
| Thioalkalivibrio | Thioalkalivibrio | Thioalkalivibrio | Thioalkalivibrio | Thioalkalivibrio |
| sp. AKL19 | sp. AKL6 | sp. AKL7 | sp. AKL8 | sp. ALE11 |
| Thioalkalivibrio | Thioalkalivibrio | Thioalkalivibrio | Thioalkalivibrio | Thioalkalivibrio |
| sp. ALE12 | sp. ALE16 | sp. ALE20 | sp. ALE31 | sp. ALE9 |
| Thioalkalivibrio | Thioalkalivibrio | Thioalkalivibrio | Thioalkalivibrio | Thioalkalivibrio |
| sp. ALgr1 | sp. ALJ1 | sp. ALJ15 | sp. ALJ16 | sp. ALJ2 |
| Thioalkalivibrio | Thioalkalivibrio | Thioalkalivibrio | Thioalkalivibrio | Thioalkalivibrio |
| sp. ALJ24 | sp. ALJ7 | sp. ALJT | sp. ALM2T | sp. ALMg11 |
| Thioalkalivibrio | Thioalkalivibrio | Thioalkalivibrio | Thioalkalivibrio | Thioalkalivibrio |
| sp. ALMg13-2 | sp. ALMg2 | sp. ALMg9 | sp. ALR17-21 | sp. ALRh |
| Thioalkalivibrio | Thioalkalivibrio | Thioalkalivibrio | Thioalkalivibrio | Thioalkalivibrio |
| sp. ALSr1 | sp. ARh3 | sp. HK1 | sp. HL-Eb18 | sulfidiphilus |
| Thioalkalivibrio | Thioalkalivibrio | Thiobacillus | Thiobacillus | Thiobacimonas |
| thiocyanodenitrificans | versutus | denitrificans | thioparus | profunda |
| Thiocapsa | Thiocapsa | Thiocapsa | Thioclava | Thioclava |
| marina | roseopersicina | sp. KS1 | atlantica | dalianensis |
| Thioclava | Thioclava | Thioclava | Thioclava | Thioclava |
| indica | marinus | nitratireducens | pacifica | sediminum |
| Thioclava | Thioclava | Thioclava | Thioclava | Thioclava |
| sp. DLFJ4-1 | sp. DLFJ5-1 | sp. ElOx9 | sp. F1Mire-8 | sp. F28-4 |
| Thioclava | Thioclava | Thioclava | Thioclava | Thioclava |
| sp. F34-6 | sp. F36-6 | sp. F36-7 | sp. F42-5 | sp. JM3 |
| Thioclava | Thioclava | Thiocystis | Thioflavicoccus | Thioflexothrix |
| sp. L04-15 | sp. SK-1 | violascens | mobilis | psekupsii |
| Thiohalocapsa | Thiohalomonas | Thiohalorhabdus | Thiohalospira | Thiolapillus |
| sp. ML1 | denitrificans | denitrificans | halophila | brandeum |
| Thiomicrospira | Thiomicrospira | Thiomicrospira | Thiomicrospira | Thiomicrospira |
| arctica | chilensis | crunogena | halophila | kuenenii |
| Thiomicrospira | Thiomicrospira | Thiomicrospira | Thiomicrospira | Thiomicrospira |
| pelophila | sp. Kp2 | sp. MA2-6 | sp. Milos-T1 | sp. Milos-T2 |
| Thiomicrospira | Thiomicrospira | Thiomonas | Thiomonas | Thiomonas |
| sp. WB1 | sp. XS5 | bhubaneswarensis | delicata | intermedia |
| Thiomonas | Thiomonas | Thiomonas | Thioploca | Thiorhodococcus |
| sp. B1 | sp. FB-6 | sp. FB-Cd | ingrica | drewsii |
| Thiorhodospira | Thiorhodovibrio | Thiothrix | Thiothrix | Thiothrix |
| sibirica | sp. 970 | caldifontis | disciformis | eikelboomii |
| Thiothrix | Thiothrix | Thiothrix | Thorsellia | Tidjanibacter |
| flexilis | lacustris | nivea | anophelis | massiliensis |
| Tilletiaria | Timonella | Tindallia | Tindallia | Tissierella |
| anomala | senegalensis | californiensis | magadiensis | creatinophila |
| Tissierella | Tissierella | Tissierellia | Tissierellia | Tissierellia |
| praeacuta | sp. P1 | bacterium KA00581 | bacterium S5-A11 | bacterium S7-1-4 |
| Tistlia | Tistrella | Tolumonas | Tolumonas | Tolypothrix |
| consotensis | mobilis | auensis | lignilytica | bouteillei |
| Tolypothrix | Tolypothrix | Tolypothrix | Tomitella | Toralaspora |
| campylonemoides | sp. NIES-4075 | sp. PCC 7601 | biformata | delbrueckii |
| Toxoplasma | Trabulsiella | Trabulsiella | Trametes | Tranquillimonas |
| gondii | guamensis | odontotermitis | versicolor | alkanivorans |
| Traorella | Tremella | Treponema | Treponema | Treponema |
| massiliensis | mesenterica | azotonutricium | berlinense | brennaborense |
| Treponema | Treponema | Treponema | Treponema | Treponema |
| bryantii | caldarium | denticola | endosymbiont of | lecithinolyticum |
| | | | Eucomonympha sp. | |
| Treponema | Treponema | Treponema | Treponema | Treponema |
| maltophilum | medium | pedis | phagedenis | porcinum |
| Treponema | Treponema | Treponema | Treponema | Treponema |
| primitia | putidum | saccharophilum | socranskii | sp. C6A8 |
| Treponema | Treponema | Treponema | Treponema | Trichococcus |
| sp. JC4 | sp. OMZ 838 | succinifaciens | vincentii | collinsii |
| Trichococcus | Trichococcus | Trichococcus | Trichoderma | Trichoderma |
| flocculiformis | palustris | pasteurii | atroviride | gamsii |
| Trichoderma | Trichoderma | Trichodesmium | Trichomonas | Trichophyton |
| reesei | virens | erythraeum | vaginalis | benhamiae |
| Trichophyton | Trichophyton | Trichormus | Trichormus | Trichosporon |
| rubrum | verrucosum | azollae | sp. NMC-1 | asahii |
| Tropheryma | Tropicibacter | Tropicibacter | Tropicibacter | Tropicimonas |
| whipplei | litoreus | multivorans | naphthalenivorans | isoalkanivorans |
| Tropicimonas | Truepera | Trueperella | Trueperella | Trypanosoma |
| sediminicola | radiovictrix | pyogenes | sp. HMSC08H06 | brucei |
| Trypanosoma | Trypanosoma | Tsuchiyaea | Tsukamurella | Tsukamurella |
| cruzi | grayi | wingfieldii | paurometabola | pseudospumae |
| Tsukamurella | Tsukamurella | Tsukamurella | Tuber | Tuberibacillus |
| pulmonis | sp. 1534 | tyrosinosolvens | melanosporum | calidus |
| Tuberibacillus | Tumebacillus | Tumebacillus | Tumebacillus | Turicella |
| sp. Marseille-P3662 | algifaecis | flagellatus | sp. AR23208 | otitidis |
| Turicella | Turicella | Turicella | Turicibacter | Turicibacter |
| sp. HMSC059E07 | sp. HMSC068H04 | sp. HMSC076G08 | sanguinis | sp. H121 |
| Turicibacter | Turicimonas | Turneriella | Type-C symbiont of | Type-D symbiont of |
| sp. HGF1 | muris | parva | Plautia stali | Plautia stali |
| Type-E symbiont of | Type-F symbiont of | Tyzzerella | Tyzzerella | Tyzzerella |
| Plautia stali | Plautia stall | nexilis | sp. An114 | sp. Marseille-P3062 |
| Uliginosibacterium | Ulvibacter | Ulvibacter | Uncinocarpus | uncultured Termite |
| gangwonense | litoralis | sp. LPB0005 | reesii | group 1 bacterium |
| unicellular | unicellular | Ureaplasma | Ureaplasma | Ureaplasma |
| cyanobacterium | cyanobacterium | canigenitalium | diversum | parvum |
| SU2 | SU3 | | | |
| Ureaplasma | Ureibacillus | Urinacoccus | Urmitella | Ustilago |
| urealyticum | thermosphaericus | massiliensis | timonensis | maydis |
| Vaccinium witches'- | Vaginella | Vagococcus | Vagococcus | Vagococcus |
| broom phytoplasma | massiliensis | fluvialis | lutrae | martis |
| Vagococcus | Vagococcus | Vanderwaltozyma | Varibaculum | Varibaculum |
| penaei | teuberi | polyspora | cambriense | sp. Marseille-P2802 |
| Varibaculum | Variovorax | Variovorax | Variovorax | Variovorax |
| timonense | boronicumulans | paradoxus | soli | sp. 770b2 |
| Variovorax | Variovorax | Variovorax | Variovorax | Variovorax |
| sp. CF079 | sp. CF313 | sp. EL159 | sp. HW608 | sp. JS1663 |
| Variovorax | Variovorax | Variovorax | Variovorax | Variovorax |
| sp. KK3 | sp. OK605 | sp. OV084 | sp. OV329 | sp. OV700 |
| Variovorax | Variovorax | Variovorax | Variovorax | Variovorax |
| sp. PAMC | sp. PDC80 | sp. Root318D1 | sp. Root411 | sp. Root434 |
| 28711 | | | | |
| Variovorax | Variovorax | Variovorax | Variovorax | Variovorax |
| sp. Root473 | sp. URHB0020 | sp. WDL1 | sp. YR216 | sp. YR266 |
| Variovorax | Variovorax | Veillonella | Veillonella | Veillonella |
| sp. YR634 | sp. YR750 | atypica | dispar | magna |
| Veillonella | Veillonella | Veillonella | Veillonella | Veillonella |
| montpellierensis | parvula | rodentium | seminalis | sp. 3_1_44 |
| Veillonella | Veillonella | Veillonella | Veillonella | Veillonella |
| sp. 6_1_27 | sp. ACP1 | sp. AS16 | sp. DNF00869 | sp. HPA0037 |
| Veillonella | Veillonella | Veillonella | Veillonellaceae | Veillonellaceae |
| sp. 1CM51a | sp. oral | tobetsuensis | bacterium DNF00626 | bacterium DNF00751 |
| | taxon 780 | | | |
| Veillonellaceae | Ventosimonas | Verminephrobacter | Verminephrobacter | Verrucomicrobia |
| bacterium KA00182 | gracilis | aporrectodeae | eiseniae | bacterium IMCC26134 |
| Verrucomicrobia | Verrucomicrobia | Verrucomicrobiaceae | Verrucomicrobiae | Verrucomicrobium |
| bacterium LP2A | bacterium SCGC | bacterium GAS474 | bacterium DG1235 | sp. 3C |
| | AAA168-F10 | | | |
| Verrucomicrobium | Verrucomicrobium | Verrucomicrobium | Verruconis | Verrucosispora |
| sp. BvORR034 | sp. BvORR106 | spinosum | gallopava | maris |
| Verrucosispora | Verticillium | Verticillium | Vibrio | Vibrio |
| sediminis | alfalfae | dahliae | aerogenes | albensis |
| Vibrio | Vibrio | Vibrio | Vibrio | Vibrio |
| alginolyticus | anguillarum | antiquarius | azureus | barjaei |
| Vibrio | Vibrio | Vibrio | Vibrio | Vibrio |
| brasiliensis | breoganii | campbellii | caribbeanicus | celticus |
| Vibrio | Vibrio | Vibrio | Vibrio | Vibrio |
| cholerae | cidicii | cincinnatiensis | coralliilyticus | crassostreae |
| Vibrio | Vibrio | Vibrio | Vibrio | Vibrio |
| cyclitrophicus | diabolicus | diazotrophicus | europaeus | ezurae |
| Vibrio | Vibrio | Vibrio | Vibrio | Vibrio |
| fluvialis | fortis | furnissii | galatheae | gazogenes |
| Vibrio | Vibrio | Vibrio | Vibrio | Vibrio |
| genomosp. F10 | genomosp. F6 | gigantis | halioticoli | harveyi |
| Vibrio | Vibrio | Vibrio | Vibrio | Vibrio |
| hepatarius | hyugaensis | ichthyoenteri | jasicida | mangrovi |
| Vibrio | Vibrio | Vibrio | Vibrio | Vibrio |
| maritimus | metoecus | metschnikovii | mexicanus | mimicus |
| Vibrio | Vibrio | Vibrio | Vibrio | Vibrio |
| mytili | natriegens | navarrensis | neptunius | nereis |
| Vibrio | Vibrio | Vibrio | Vibrio | Vibrio |
| nigripulchritudo | ordalii | orientalis | ostreicida | owensii |
| Vibrio | Vibrio | Vibrio | Vibrio | Vibrio |
| pacinii | palustris | panuliri | parahaemolyticus | phage phi 1 |
| Vibrio | Vibrio | Vibrio | Vibrio | Vibrio |
| phage phi 3 | ponticus | proteolyticus | quintilis | renipiscarius |
| Vibrio | Vibrio | Vibrio | Vibrio | Vibrio |
| rhizosphaerae | rotiferianus | ruber | rumoiensis | sagamiensis |
| Vibrio | Vibrio | Vibrio | Vibrio | Vibrio |
| scophthalmi | shilonii | sinaloensis | sonorensis | sp. 10N.222.47.A9 |
| Vibrio | Vibrio | Vibrio | Vibrio | Vibrio |
| sp. 10N.261.45.E1 | sp. 16 | sp. 2521-89 | sp. AND4 | sp. B183 |
| Vibrio | Vibrio | Vibrio | Vibrio | Vibrio |
| sp. CA-1004 | sp. ECSMB14105 | sp. ECSMB14106 | sp. EJY3 | sp. ER1A |
| Vibrio | Vibrio | Vibrio | Vibrio | Vibrio |
| sp. HI00D65 | sp. J2-17 | sp. J2-26 | sp. J2-31 | sp. M12-1144 |
| Vibrio | Vibrio | Vibrio | Vibrio | Vibrio |
| sp. MEBiC08052 | sp. MED222 | sp. N418 | sp. OY15 | sp. Q67 |
| Vibrio | Vibrio | Vibrio | Vibrio | Vibrio |
| sp. qd031 | sp. RC586 | sp. S234-5 | sp. S457-15 | sp. S512-13 |
| Vibrio | Vibrio | Vibrio | Vibrio | Vibrio |
| sp. V08_P9A1T1 | sp. V11_P1A41T118 | sp. V19_P1S1T109 | sp. Vb278 | sp. VPAP30 |
| Vibrio | Vibrio | Vibrio | Vibrio | Vibrio |
| sp. ZF57 | spartinae | splendidus | tasmaniensis | toranzoniae |
| Vibrio | Vibrio | Vibrio | Vibrio | Vibrionaceae |
| tritonius | tubiashii | vulnificus | xiamenensis | bacterium |
| | | | | NSCS20N07D |
| Vibrionales | Virgibacillus | Virgibacillus | Virgibacillus | Virgibacillus |
| bacterium | alimentarius | chiguensis | dakarensis | dokdonensis |
| SWAT-3 | | | | |
| Virgibacillus | Virgibacillus | Virgibacillus | Virgibacillus | Virgibacillus |
| halodenitrificans | necropolis | pantothenticus | proomii | salinus |
| Virgibacillus | Virgibacillus | Virgibacillus | Virgibacillus | Virgibacillus |
| senegalensis | siamensis | soli | sp. 6R | sp. 7505 |
| Virgibacillus | Virgibacillus | Virgibacillus | Virgibacillus | Viridibacillus |
| sp. IO3-P2-C2 | sp. LM2416 | sp. SK37 | subterraneus | arvi |
| Vitellibacter | Vitreoscilla | Vitreoscilla | Vitreoscilla | Vogesella |
| aquimaris | filiformis | sp. SN6 | stercoraria | sp. EB |
| Vogesella | Vulcanibacillus | Vulcanisaeta | Vulcanisaeta | Vulgatibacter |
| sp. LIG4 | modesticaldus | distributa | sp. EB80 | incomptus |
| Waddlia | Wallemia | Wallemia | Weeksella | Weeksella |
| chondrophila | ichthyophaga | mellicola | massiliensis | sp. HMSC059D05 |
| Weeksella | Weissella | Weissella | Weissella | Weissella |
| virosa | bombi | ceti | cibaria | confusa |
| Weissella | Weissella | Weissella | Weissella | Weissella |
| halotolerans | hellenica | jogaejeotgali | kandleri | koreensis |
| Weissella | Weissella | Weissella | Weissella | Weissella |
| minor | oryzae | paramesenteroides | sp. DD23 | viridescens |
| Wenxinia | Wenxinia | Wenyingzhuangia | Wenyingzhuangia | Wenzhouxiangella |
| marina | saemankumensis | fucanilytica | marina | marina |
| Wheat blue dwarf | Wickerhamomyces | Wickerhamomyces | Wigglesworthia | Williamsia |
| phytoplasma | anomalus | ciferrii | glossinidia | herbipolensis |
| Williamsia | Williamsia | Williamsia | Williamsia | Williamsia |
| muralis | sp. 1135 | sp. 1138 | sp. D3 | sp. Leaf354 |
| Williamsia | Winogradskyella | Winogradskyella | Winogradskyella | Winogradskyella |
| sterculiae | jejuensis | psychrotolerans | sp. J14-2 | sp. PC-19 |
| Winogradskyella | Winogradskyella | Winogradskyella | Winogradskyella | Woeseia |
| sp. PG-2 | sp. RHA_55 | sp. WNB302 | thalassocola | oceani |
| Wohlfahrtiimonas | Wohlfahrtiimonas | Wohlfahrtiimonas | Wohlfahrtiimonas | Wolbachia |
| chitiniclastica | larvae | sp. 34C10-3-10 | sp. G9077 | endosymbiont of |
| | | | | Cimex |
| | | | | lectularius |
| Wolbachia | Wolbachia | Wolbachia | Wolbachia | Wolbachia |
| endosymbiont of | endosymbiont of | endosymbiont of | endosymbiont of | pipientis |
| Drosophila | Laodelphax | Trichogramma | Wuchereria | |
| simulans | striatella | pretiosum | bancrofti | |
| Wolinella | Woodsholea | Xanthobacter | Xanthobacter | Xanthobacter |
| succinogenes | maritima | autotrophicus | sp. 126 | sp. 91 |
| Xanthomarina | Xanthomonas | Xanthomonas | Xanthomonas | Xanthomonas |
| gelatinilytica | albilineans | alfalfae | arboricola | axonopodis |
| Xanthomonas | Xanthomonas | Xanthomonas | Xanthomonas | Xanthomonas |
| bromi | campestris | cannabis | cassavae | citri |
| Xanthomonas | Xanthomonas | Xanthomonas | Xanthomonas | Xanthomonas |
| euvesicatoria | floridensis | fragariae | fuscans | gardneri |
| Xanthomonas | Xanthomonas | Xanthomonas | Xanthomonas | Xanthomonas |
| hortorum | maliensis | massiliensis | nasturtii | oryzae |
| Xanthomonas | Xanthomonas | Xanthomonas | Xanthomonas | Xanthomonas |
| perforans | phaseoli | pisi | retroflexus | sacchari |
| Xanthomonas | Xanthomonas | Xanthomonas | Xanthomonas | Xanthomonas |
| sp. GPE 39 | sp. Leaf131 | sp. Leaf148 | sp. Mitacek01 | sp. MUS 060 |
| Xanthomonas | Xanthomonas | Xanthomonas | Xanthomonas | Xanthomonas |
| sp. NCPPB1128 | sp. SHU166 | sp. SHU199 | sp. SHU308 | translucens |
| Xanthomonas | Xanthomonas | Xanthomonas | Xenococcus | Xenophilus |
| vasicola | vesicatoria | virus OP2 | sp. PCC 7305 | azovorans |
| Xenophilus | Xenorhabdus | Xenorhabdus | Xenorhabdus | Xenorhabdus |
| sp. AP218F | beddingii | bovienii | cabanillasii | doucetiae |
| Xenorhabdus | Xenorhabdus | Xenorhabdus | Xenorhabdus | Xenorhabdus |
| eapokensis | griffiniae | hominickii | innexi | japonica |
| Xenorhabdus | Xenorhabdus | Xenorhabdus | Xenorhabdus | Xenorhabdus |
| khoisanae | koppenhoeferi | mauleonii | nematophila | poinarii |
| Xenorhabdus | Xenorhabdus | Xenorhabdus | Xenorhabdus | Xiangella |
| sp. NBAII XenSa04 | szentirmaii | thuongxuanensis | vietnamensis | phaseoli |
| Xylanimonas | Xylella | Xylella | Xylona | Xylophilus |
| cellulosilytica | fastidiosa | taiwanensis | heveae | ampelinus |
| Xylophilus | Yangia | Yangia | Yaniella | Yarrowia |
| sp. Leaf220 | pacifica | sp. CCB-MM3 | halotolerans | lipolytica |
| Yersinia | Yersinia | Yersinia | Yersinia | Yersinia |
| aldovae | aleksiciae | bercovieri | enterocolitica | entomophaga |
| Yersinia | Yersinia | Yersinia | Yersinia | Yersinia |
| frederiksenii | intermedia | kristensenii | massiliensis | mollaretii |
| Yersinia | Yersinia | Yersinia | Yersinia | Yersinia |
| nurmii | pekkanenii | pestis | phage L-413C | pseudotuberculosis |
| Yersinia | Yersinia | Yersinia | Yokenella | Yonghaparkia |
| rohdei | ruckeri | wautersii | regensburgei | sp. Root332 |
| Yonghaparkia | Youngiibacter | Yuhushiella | Zavarzinella | Zetaproteobacteria |
| sp. Soil809 | fragilis | deserti | formosa | bacterium TAG-1 |
| Zhongshania | Zhouia | Zobellella | Zobellia | Zobellia |
| aliphaticivorans | amylolytica | denitriflcans | galactanivorans | uliginosa |
| Zoogloea | Zooshikella | Zunongwangia | Zunongwangia | Zunongwangia |
| sp. LCSB751 | ganghwensis | atlantica | mangrovi | profunda |
| Zygosaccharomyces | Zymobacter | Zymomonas | Zymoseptoria | |
| rouxii | palmae | mobilis | tritici | |

All scientific and technical terms used in this application have meanings commonly used in the art unless otherwise specified.

As used herein, the term “subject” includes humans and non-human animals. The term “non-human animal” includes all vertebrates, e.g., mammals and non-mammals, such as non-human primates, horses, sheep, dogs, cows, pigs, chickens, and other veterinary subjects and test animals.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the terms “sample” and “biological sample” refer to any sample suitable for the methods provided by the present invention. A sample of cells can be any sample, including, for example, gut or fecal sample obtained by non-invasive or invasive techniques such as biopsy of a subject. In one embodiment, the term “sample” refers to any preparation derived from fecal matter or gut tissue of a subject. For example, a sample of cells obtained using the non-invasive method described herein can be used to isolate nucleic acid molecules or proteins for the methods of the present invention.

In embodiments, analysis can be of any nucleic acid, including DNA, RNA, cDNA, miRNA, mtDNA, single or double-stranded. This nucleic acid can be of any length, as short as oligos of about 5 bp to as long a megabase or even longer. As used herein, the term “nucleic acid molecule” means DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. For methods that analyze expression of a gene, the nucleic acid isolated from a sample is typically RNA.

A single-stranded nucleic acid molecule is “complementary” to another single-stranded nucleic acid molecule when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

As used herein “hybridization” refers to the process by which a nucleic acid strand joins with a complementary strand through base pairing. Hybridization reactions can be sensitive and selective so that a particular sequence of interest can be identified even in samples in which it is present at low concentrations. In an in vitro situation, suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

As used herein, the term “microbiome” refers to microorganisms, including bacteria, viruses, and fungi, archaea, protozoa, amoeba, or helminths that inhabit the gut of the subject.

As used herein, the terms microbial, microbe, or microorganism refer to any microscopic organism including prokaryotes or eukaryotes, spores, bacterium, archeaebacterium, fungus, virus, or protist, unicellular or multicellular.

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

Methods for data analysis according to various aspects of the present invention may be implemented in any suitable manner, for example using a computer program operating on the computer system. An exemplary analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The analysis system according to various aspects of the present invention and its various elements provide functions and operations to facilitate microbiome analysis, such as data gathering, processing, analysis, reporting and/or diagnosis. The present analysis system maintains information relating to microbiomes and samples and facilitates analysis and/or diagnosis. For example, in the present embodiment, the computer system executes the computer program, which may receive, store, search, analyze, and report information relating to the microbiome. The computer program may comprise multiple modules performing various functions or operations, such as a processing module for processing raw data and generating supplemental data and an analysis module for analyzing raw data and supplemental data to generate a models and/or predictions.

The analysis system may also provide various additional modules and/or individual functions. For example, the analysis system may also include a reporting function, for example to provide information relating to the processing and analysis functions. The analysis system may also provide various administrative and management functions, such as controlling access and performing other administrative functions.

The following examples are provided to further illustrate the embodiments of the present invention, but are not intended to limit the scope of the invention. While they are typical of those that might be used, other procedures, methodologies, or techniques known to those skilled in the art may alternatively be used.

To the extent necessary to understand or complete the disclosure of the present invention, all publications, patents, and patent applications mentioned herein are expressly incorporated by reference therein to the same extent as though each were individually so incorporated.

### EXAMPLES

### Example 1

### Microbiome Analysis

Subjects were subject to microbiome analysis to produce a microbiome profile. Subject stool samples were collected using a collection card/specialized paper. The collection card/specialized paper serves to preserve the DNA of all species of interest, including phages and viruses. A proprietary DNA extraction protocol was used to extract DNA from bacteria, fungi, viruses, phages, archaea and helminths in the stool sample. The DNA was subjected to shotgun sequencing. Whole Genome Shotgun sequencing was found to be more sensitive than 16S sequencing. The data resulting from the whole genome shotgun sequencing was analyzed to determine the presence of bacteria, fungi, viruses, phages, archaea and/or helminths in the sample. An in house tool along with an in house reference genome is used to identify the species of organism present in a sample and to compare abundance levels to the general population. The in house tool is built on open source and in house bioinformatics software. The in house reference genome was built on public data bases with in house curation.

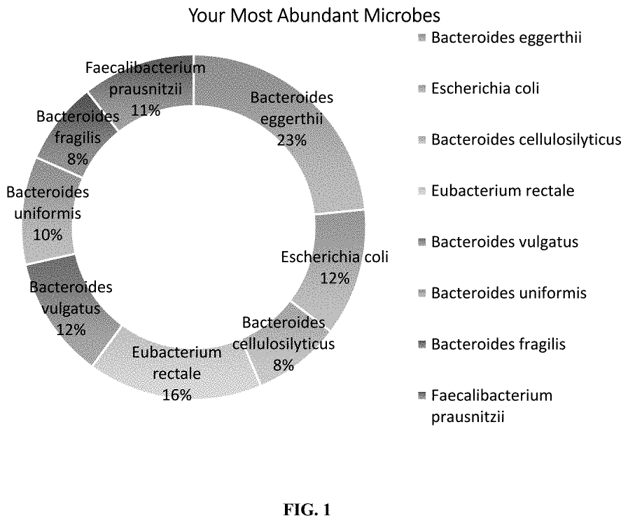

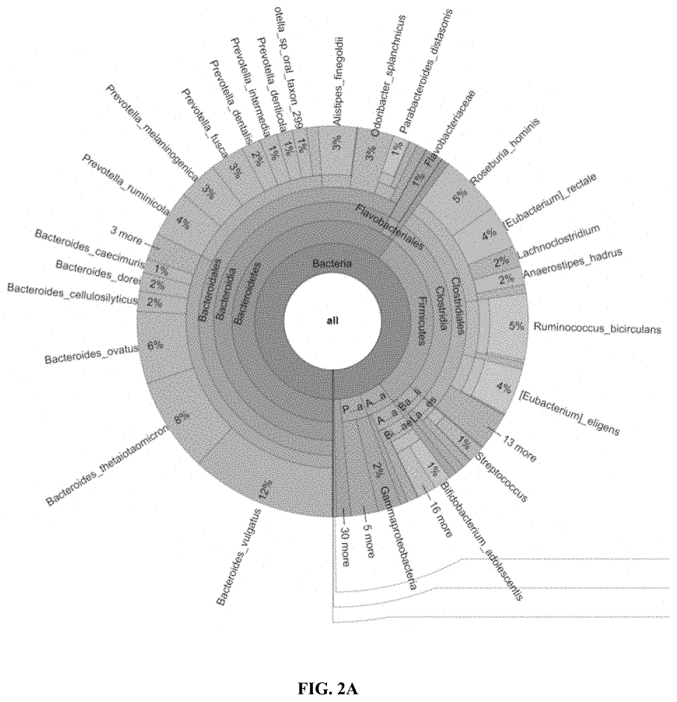

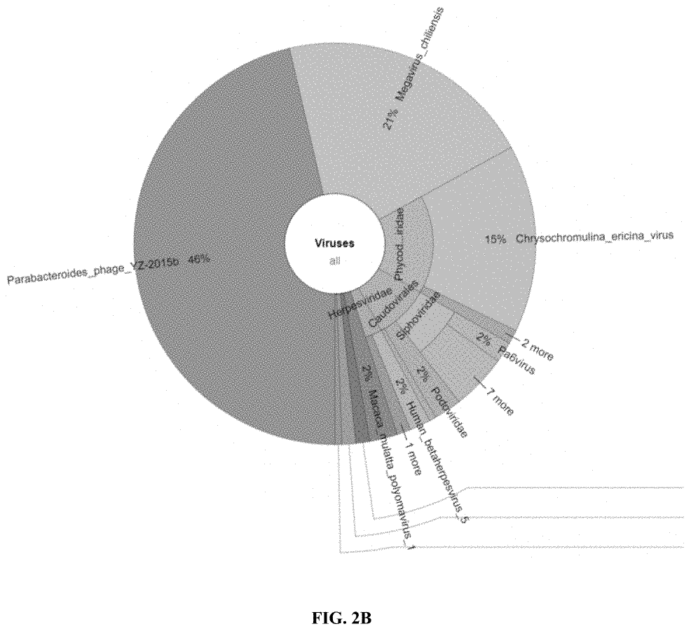

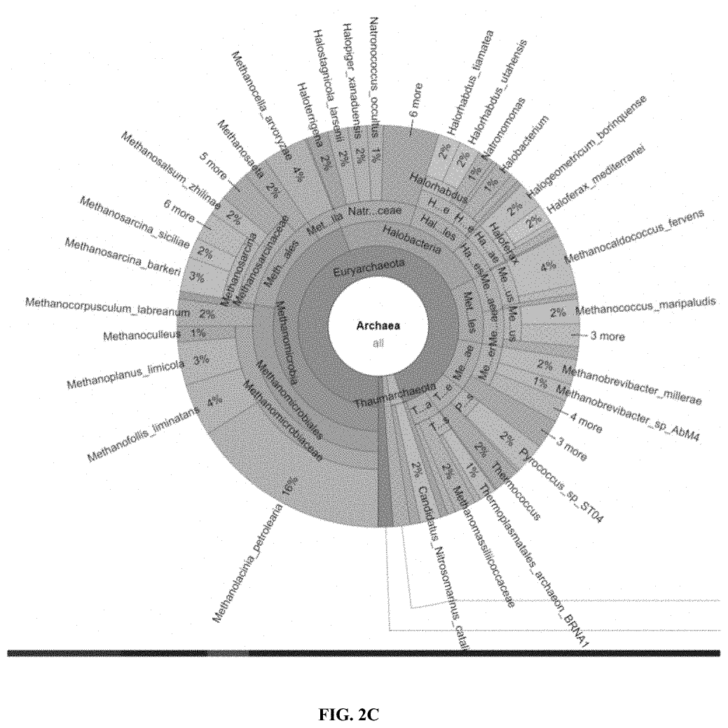

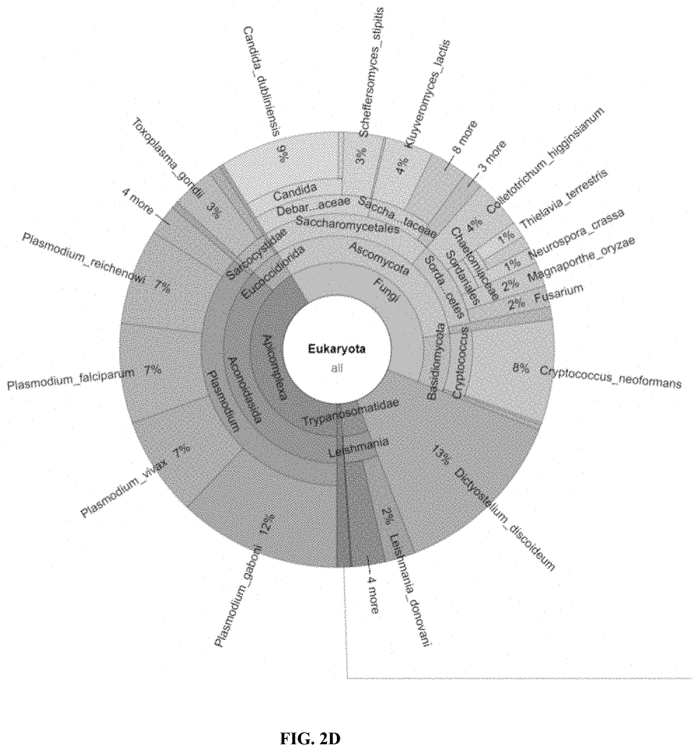

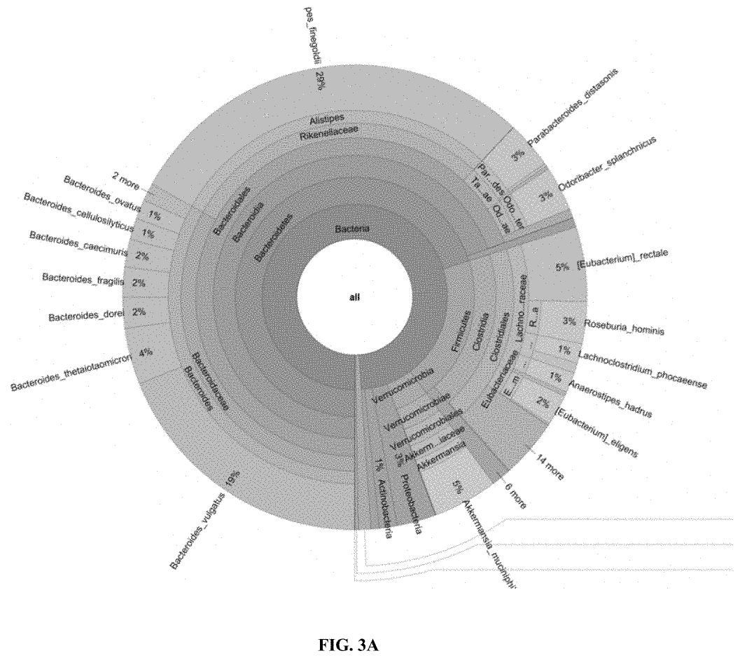

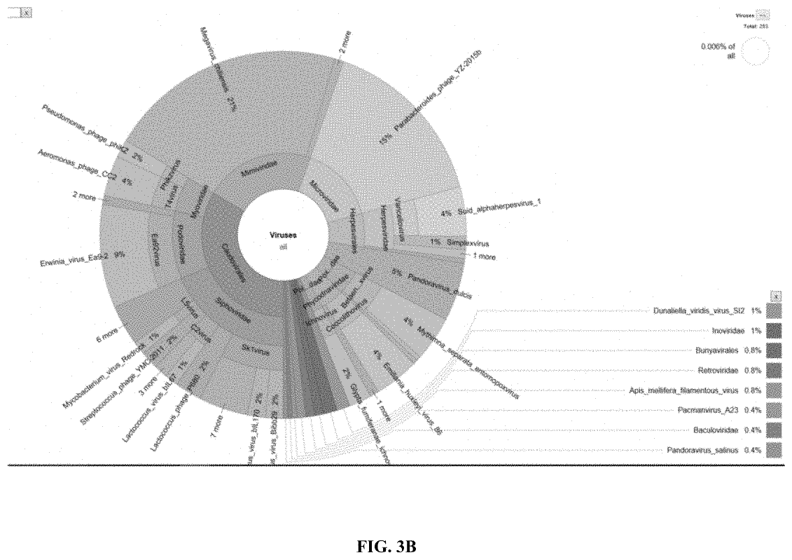

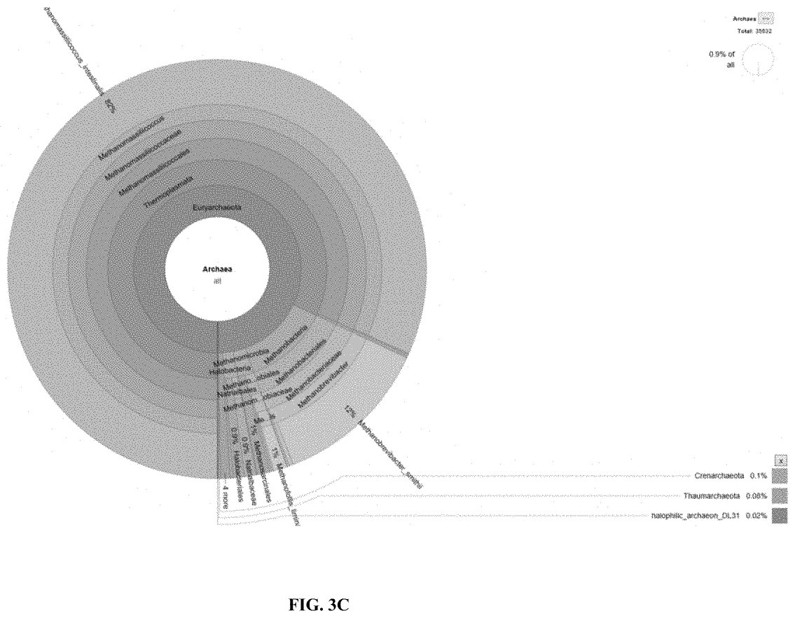

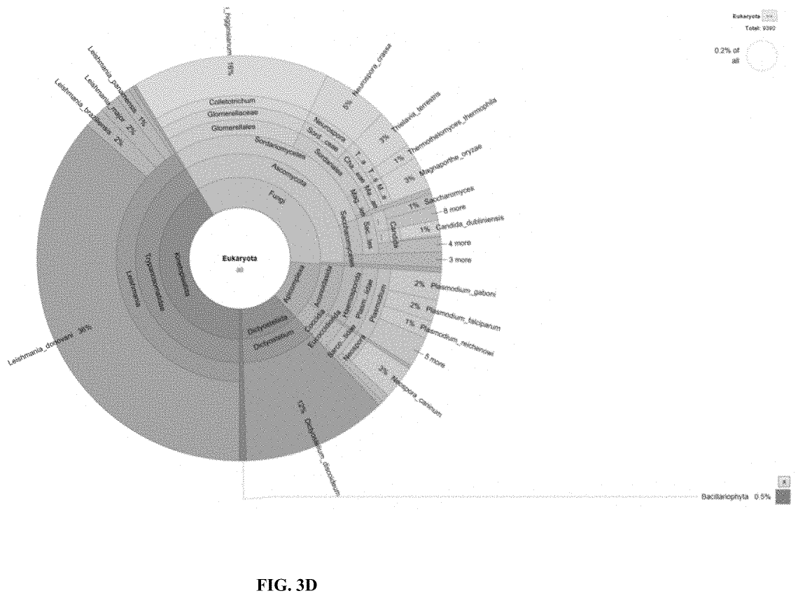

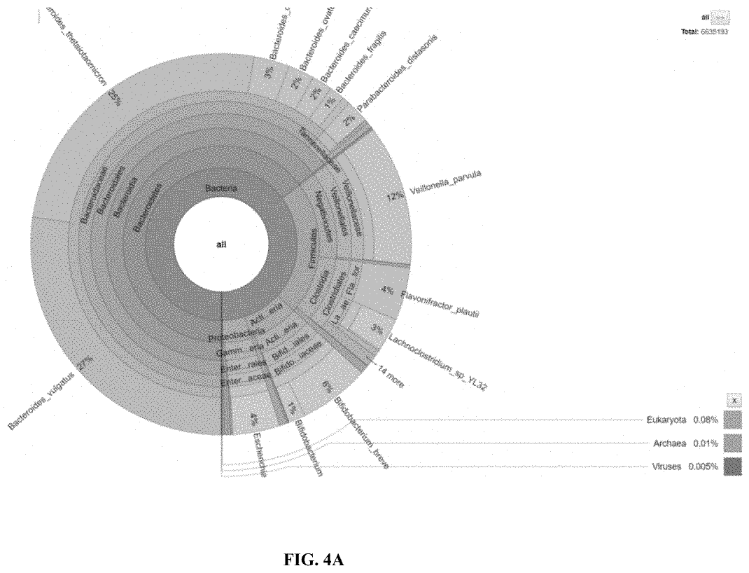

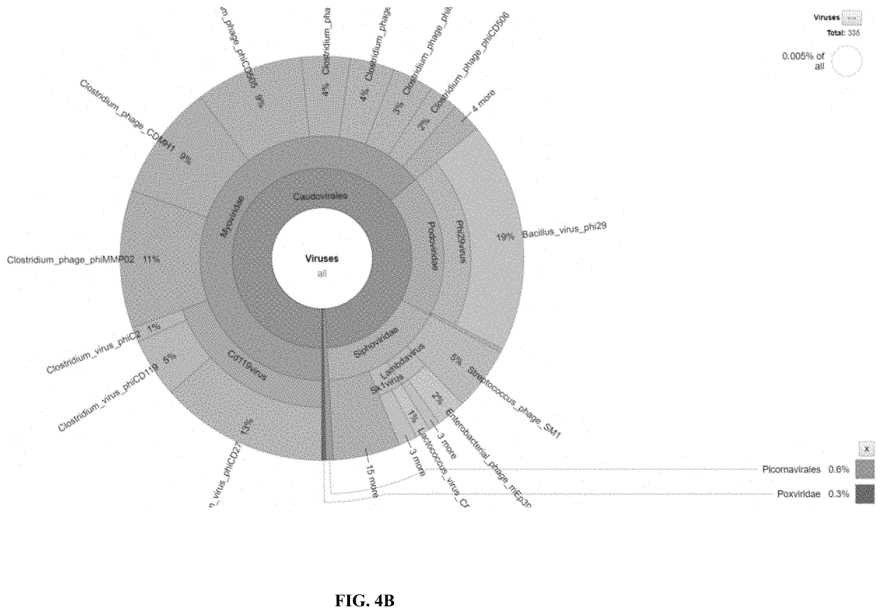

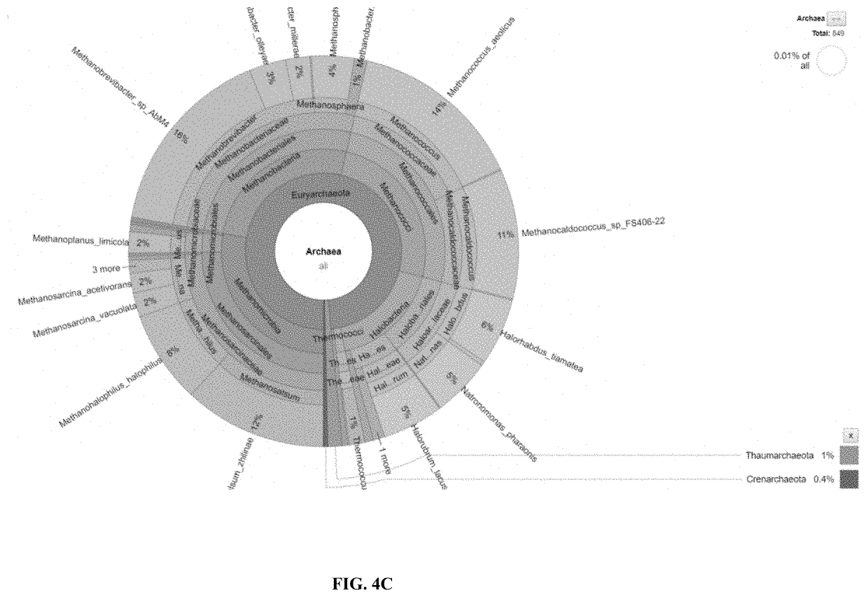

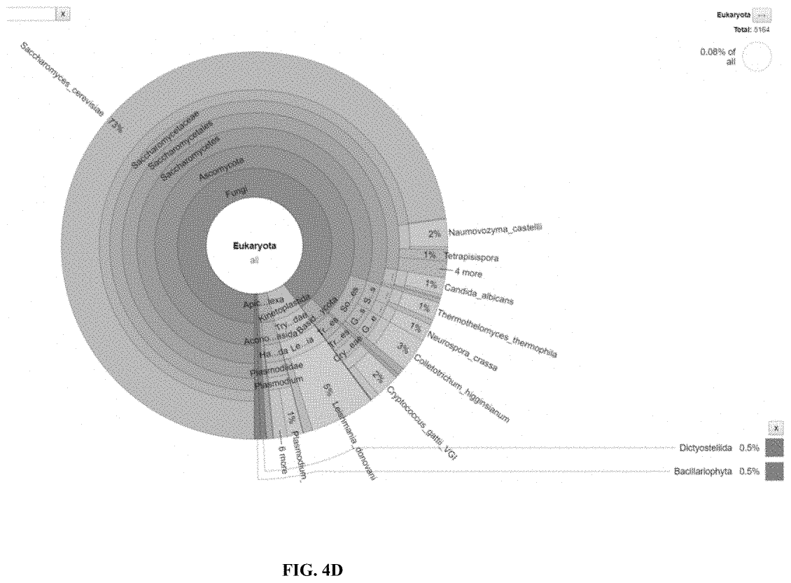

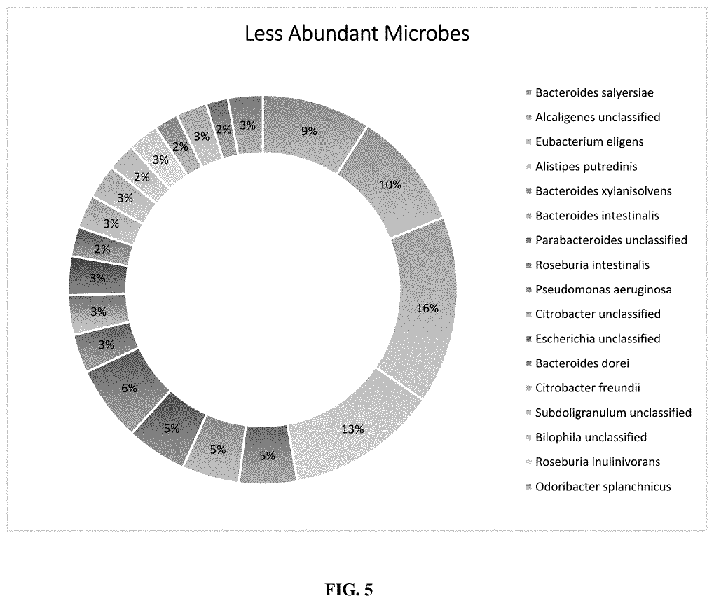

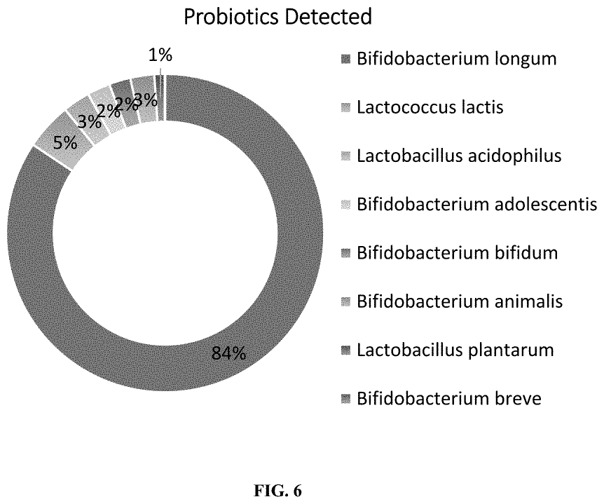

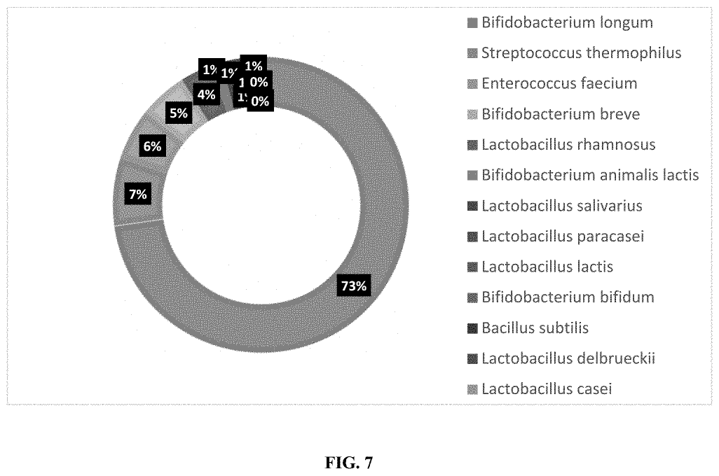

FIG. 1 shows the high prevalence organisms of a microbiome signature of a subject with a high protein diet, who is >50 years old, and is supplement user. Several different species of viral, archaea and eukaryotic organisms were identified in the samples as well as determining the abundance levels of the organisms (FIGS. 2-4). FIG. 2 shows of high prevalence organisms (bacteria, viruses, phages and aschaea) of a microbiome signature of a subject with a high carbohydrate diet, who is 18-50 years old, and has a vegetarian diet. FIG. 3 shows of high prevalence organisms (bacteria, viruses, phages and aschaea) of a microbiome signature of a subject with a high carbohydrate diet, who is 18-50 years old, and has a non-vegetarian diet. FIG. 4 shows of high prevalence organisms (bacteria, viruses, phages and aschaea) of a microbiome signature of a subject with a high dairy protein diet, who is 0-2 years old, and has a vegetarian non-nursing diet. Examples of viruses identified in the samples include Chrysochromulina ericina virus and Megavirus chilensis. Examples of archaea organisms identified in the samples include Methanolinea petrolera and Haloferax mediterranei. Examples of eukaryotic organisms identified in the samples include Cryprococcus neoformans and Plasmodium gaboni. FIG. 5 shows least prevalent organisms and identification of opportunistic pathogens of a microbiome signature of a subject. Further, different species of probiotics such as Bifidobacterium longum and Lactobacillus acidophilus were identified in subject samples (FIGS. 6-7).

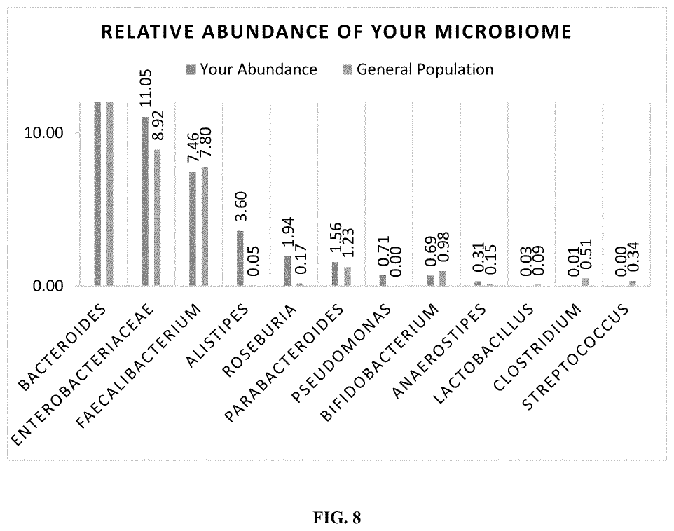

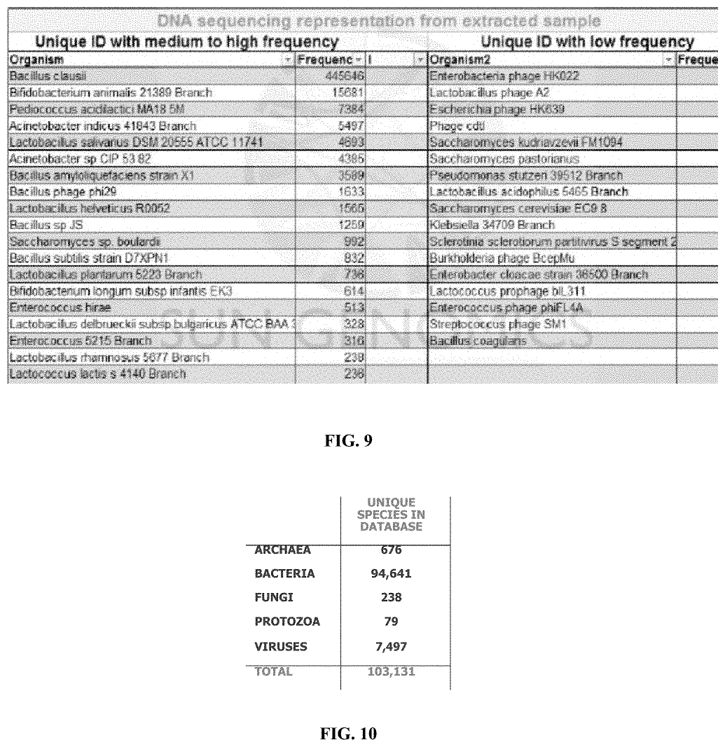

A comparison was performed to show the differences between the relative abundance of microbes identified in a subject sample with the general population (FIG. 8). As is shown in FIG. 8 this subjects microbiome was substantially similar to that of the general population with the biggest difference in the levels of Roseburia. Further, the analysis was performed to identify the most and least frequent microbes identified in a sample from a subject as well as the number of different species of archaea, bacteria, fungi, protozoa and viruses that were identified from samples (FIGS. 9 and 10). FIG. 9 shows the microbiome profile from a dietary supplement mixed culture. FIG. 10 shows the classification of unique species of various microbes stored in the database of the invention.

### Example 2

### Specific Subject Examples of Microbiome Analysis

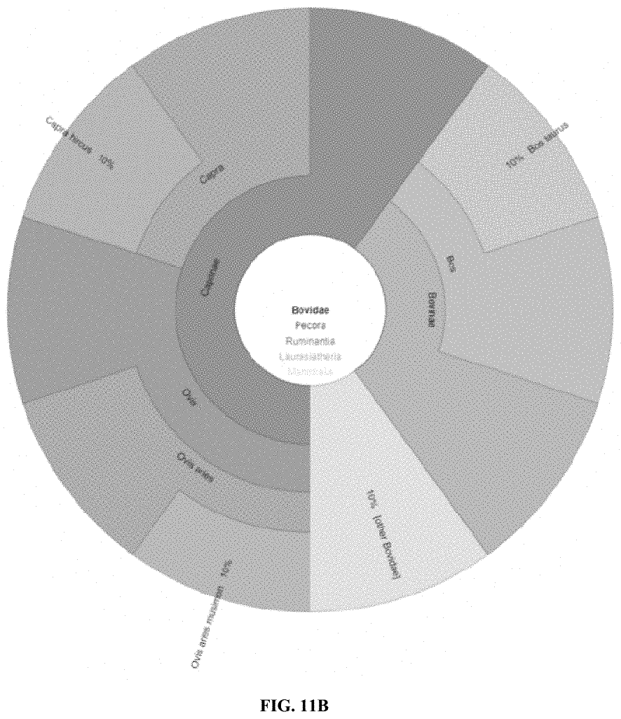

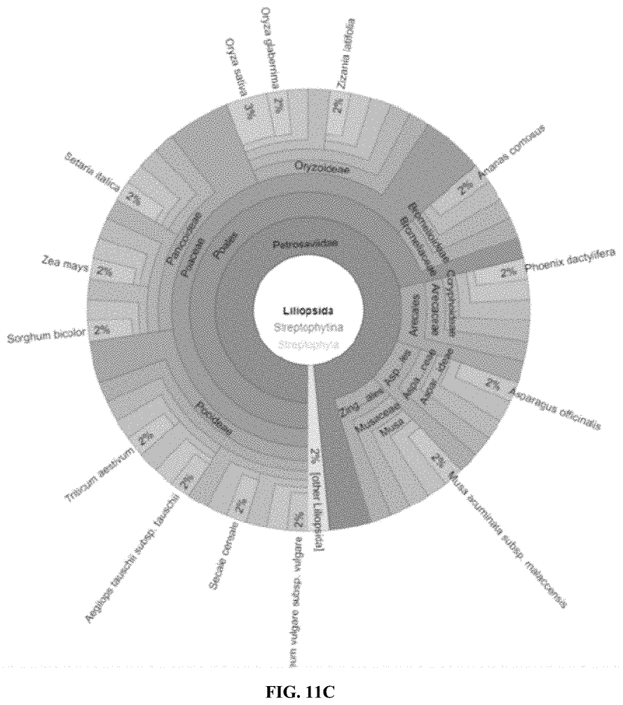

One subject submitted a survey prior to providing a sample for microbiome analysis. The subject was of Asian descent, with a BMI of 24.7 and a self-reported relatively high energy level (i.e 4/5). The subject did not consume protein shakes of other nutritional supplements. The subject reported having a low alcohol consumption level (1 glass of wine/beer a week) and didn't smoke. The subject's diet consisted of chicken, pork, beef, rice, vegetables and seafood. The subject's sample was analyzed to identify Mollusca, Bovidae and Liliopsida organisms (FIGS. 11A-C). Several types of Mollusca organisms were identified related to seafood consumption including Mytilus galloprovincialis and Mizuhopecten yessoensis (FIG. 11A). Several types of Bovidae organisms were identified related to meat consumption including Ovis aries musimon and Bos taurus (FIG. 11B). Several types of Lilopsida organisms were identified related to grain consumption including Asparagus officinalis and Ananas comosus (FIG. 11C).

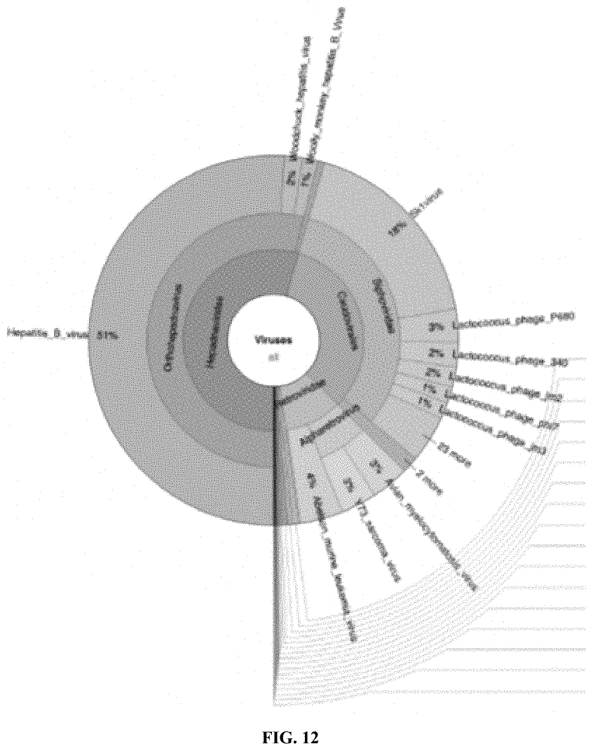

One subject was being evaluated as a normal control subject. The subject was diagnosed with a latent Hepatitis B infection using the disclosed methods (FIG. 12). This diagnosis was later confirmed by a blood test.

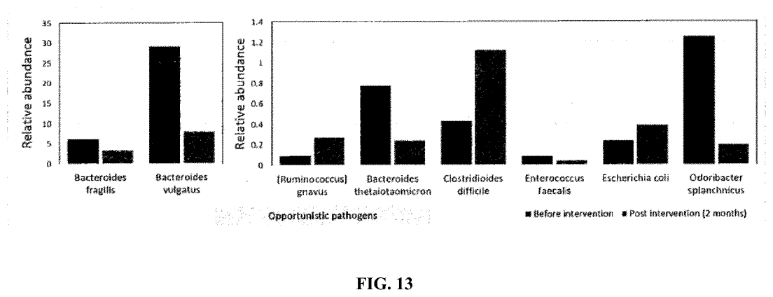

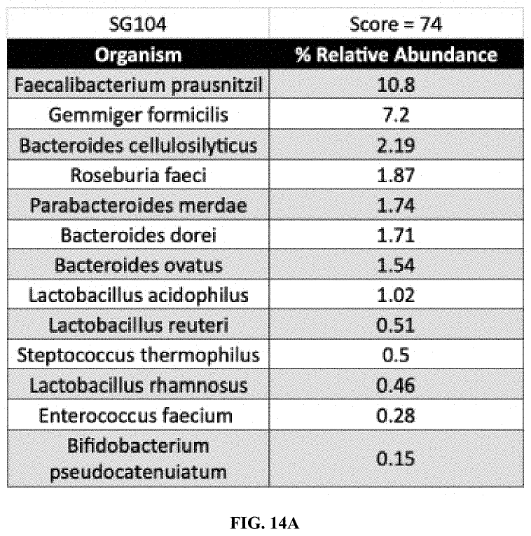

Another subject was diagnosed with small intestinal bacterial overgrowth (SIBO). The subject's sample was analyzed before and two months after treatment with antibiotics (FIG. 13). The results indicated decreased levels of Bacteroides Bacteroides vulgatus, Bacteroides thetaiotaomicron, Odoribactor splanchnicus and increased levels of Ruminococcus gnavus, Clostridoides difficile and E. coli following antibiotic treatment.

### Example 3

### Use and Development of Personalized Probiotics

Isolation and characterization of Novel probiotics and delivery back to individuals as single isolates or in combination to restore the gut, blood, skin, lung, genitals, oral microbiome to an improved health status and reduce symptoms associated with chronic conditions, disease, or disorders and utilization of molecular, biochemical, immunological, or other chemistry techniques for detection of the gut, blood, skin, lung, genitals, oral (microbial ecosystems) composition prior to individualized therapy, supplement, therapeutic, or medical product. The use of probiotic strains, prebiotics, postbiotics, herbs (such as turmeric, curcumin, flaxseed), herbal extracts (fibers, inulin, starches), or other biomolecules to reduce the concentration of metabolites, probiotics, or molecules for the improvement of health. In addition to the use of these known probiotic strains, Sun Genomics herein describes the use of new strains of gut bacteria to restore conditions and profiles of the microbial ecosystems that would be non-favorable to the host (human, animal, or other organism). Below is a table list of the organism of use, the combination of which it may be used a part of or by itself, not limited to but as example the strain ID, the profiles that has defined this as healthy and the profiles types it can affect to restore gut health, chronic conditions, wellness, disease, or disorders. One mechanism by which this is accomplished is through tight cell junction repair of the cells that line the intestinal system, this repair is mediated through molecules released by the intestinal system or microbes within the intestinal system. Such molecules include short chain fatty acids (SCFA) known as resistance starches. Resistant starch is especially associated with one type of SCFA, called butyrate, which is protective of colon cells and associated with less genetic damage butyrate also protects the cells in other ways. This is one of the real strengths of resistant starch over oligosaccharides and soluble fiber. Their fermentation does produce butyrate, but not at the levels of resistant starch. The invention herein describes the ability to precisely determine at the species and strain level the organism of the microbial ecosystem and modulate its concentration through the use of other microbes that have been isolated and/or purified from a microbial ecosystem along with or along with other biomolecules such as prebiotics, postbiotics, herbs, or extracts. Specifically, we modulate particular microbiome profiles using the organisms and molecules alone or in combination with other molecules and organisms, where any of the combination microbe may also be the main strain used alone or in combination. The below table lists common strains that may be used at the lead strain as an example and extends beyond this to other strains yet unpublished to the public databases. Key to this process is the whole genome sequencing, 16s/18s/ITS sequencing, PCR, or other molecular or biochemical methods that analyze the proteins, DNA, or RNA of the sample and assign the identification and taxonomy of the organism from kingdom to species and strain. This system is typically called a bioinformatics platform which we have previously described in U.S. Pat. No. 10,428,370. This bioinformatic system can then be used along with a database of individuals that serve as a reference set to help define the healthy normal profile. The reference set then serves as the goal to modulate a microbiome toward. The healthy normal profile of an individual is defined here in FIG. 1 as an example of an omnivore, however other profiles such as that of a herbivore, or predominate carnivore may be represented as a healthy normal. Diets such as Mediterranean, vegan, vegetarian (and all derivatives thereof), paleo, keto, aktins, slow carb, or geographical regions, etc may also have profiles that define healthy normal from which are used as reference to modulate the microbiome with single strains or strains in combination of 1 or more or with multiple combinations of groups or a single group with a single strain or group of strains form any other combination.

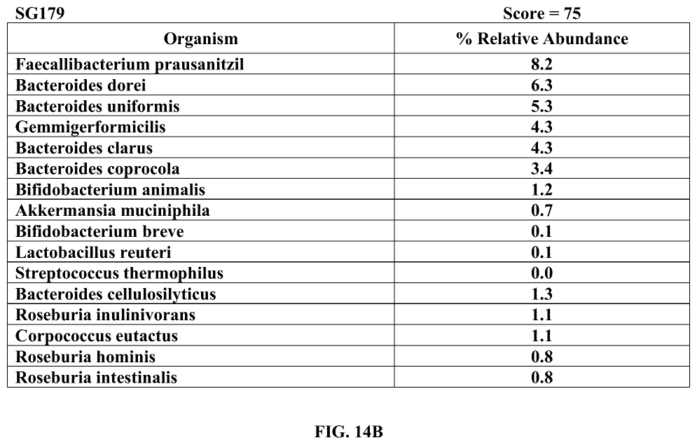

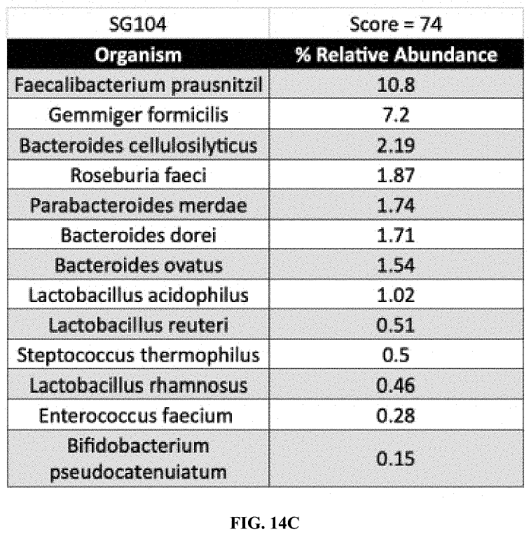

Subject samples were collected and processed as described previously. Subject samples were analyzed to develop a microbiome profile of the species and levels of organisms. The profiles were compared with a healthy reference microbiome profile to identify any imbalances in the microbiome. Personalized probiotic combinations, including new strains of bacteria, were developed to correct these imbalances. A healthy reference profile (i.e. a healthy gut microbiome) was defined as having Faecelibacterium above 10%; Gemmiger formicillis above 2%; Bacteroides vulgatus below 12% and Bacteroides fragilis between 1-5%; along with Bacteroides celluosilyticus, faecis and finegoldi above 1% or more each with lots of probiotic diversity of organisms above 0.1% including Lactobacillus gasseri, acidophilus; lactis, reuteri, rhamnous, pseudocatenulatum along with Rosburia faecis, inteninalis, or inulinlvorans with at least 1% or higher or aggregate above 2%, with Eubacterium species such as rectale, hallii, eligens at 1% or greater. Devoid or reduced Enterobacteriaceae family organisms (below 2%) such as Escherichia, Shigella, Salmonella, Candidatus, and Klebsiella genus organisms. Devoid or reduced Pseudomonas genus (below 1%) organisms. Examples of a microbiome profiles that were used to define the healthy reference profile are shown in FIGS. 14A-C.

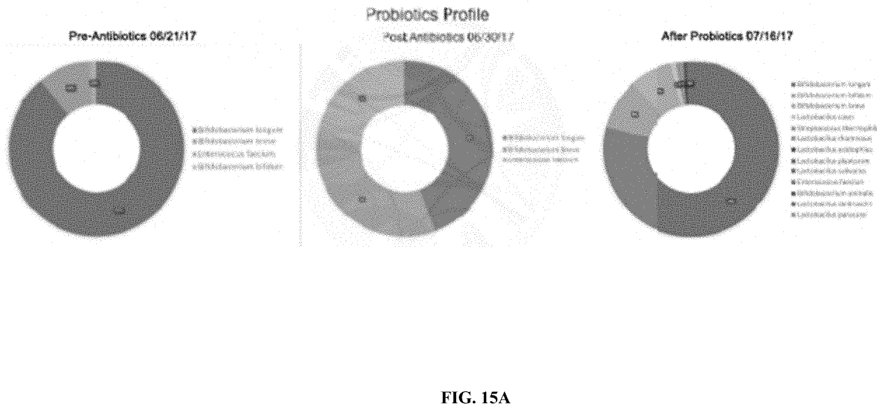

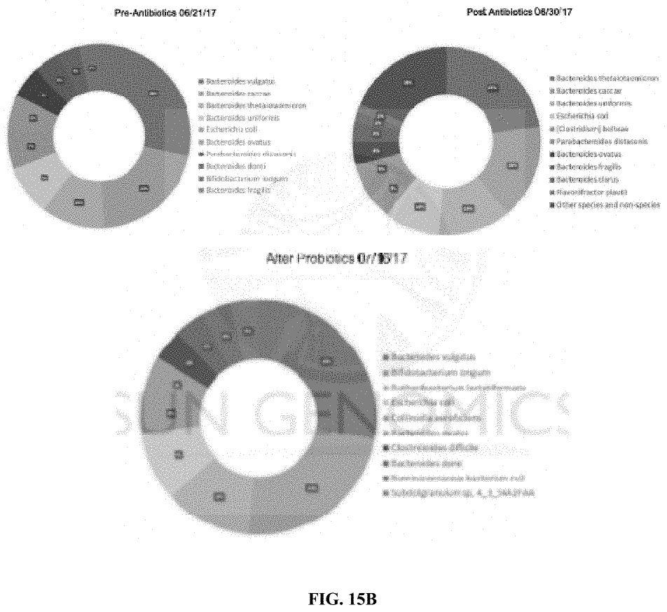

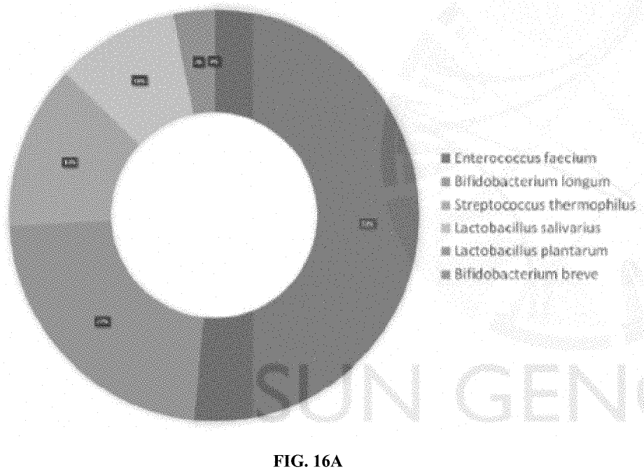

One subject was analyzed after antibiotic treatment. The bacterium Bifidobacterium bilidus is commonly transferred from mother to infant in vaginal births and is key to establishing and colonizing infant microbes. Bifidobacterium infanis was not found prior to or after antibiotic treatment as this subject was not nursing and these organisms transfer through breast milk to the infant and is key to increase nutritional benefits from mother's milk. Post antibiotic and probiotic analysis showed that the treatment fully restored the probiotic profile to help reduce colonization of opportunistic pathogens (FIG. 15).

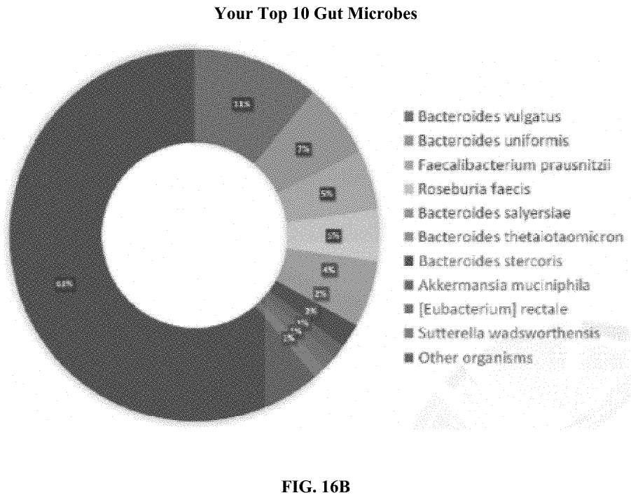

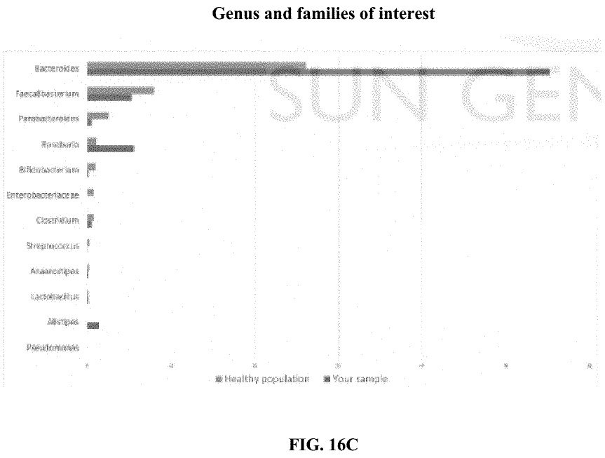

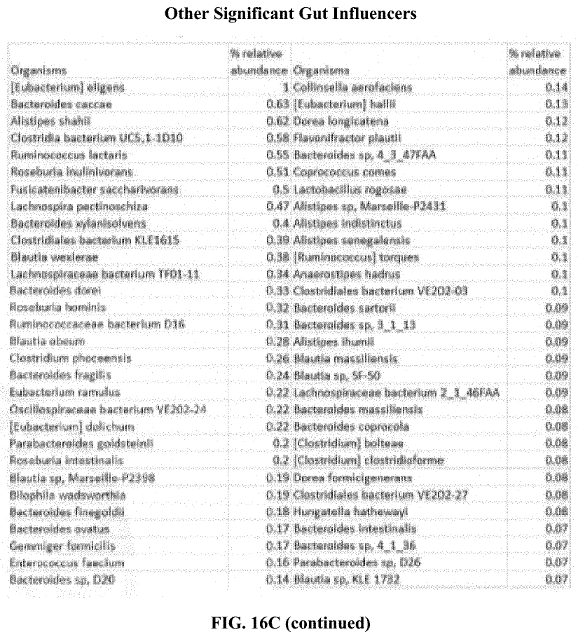

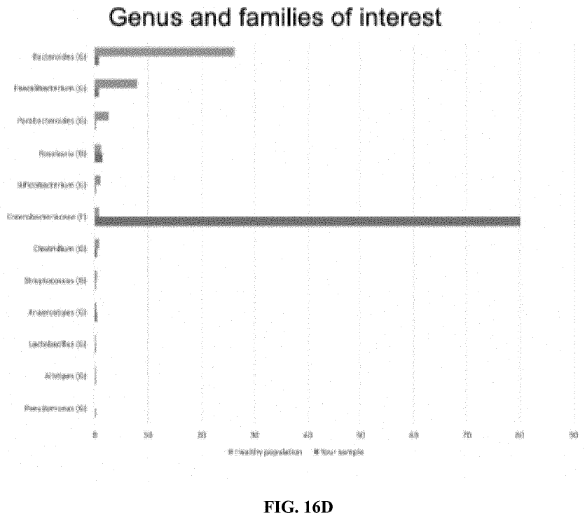

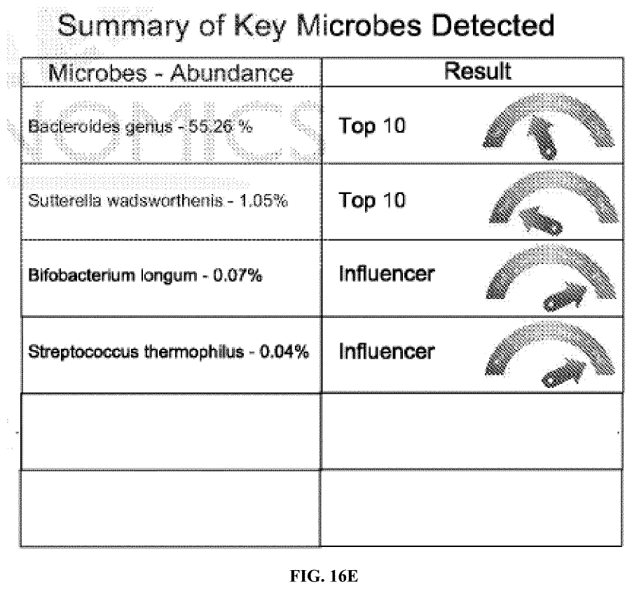

Another subject was found to have more than one probiotic organism in the gut influencer group indicating that the subject has some immunity to parasitic microbes. A probiotic regimen was formulated to maximize the subject's probiotic profile (FIG. 16).

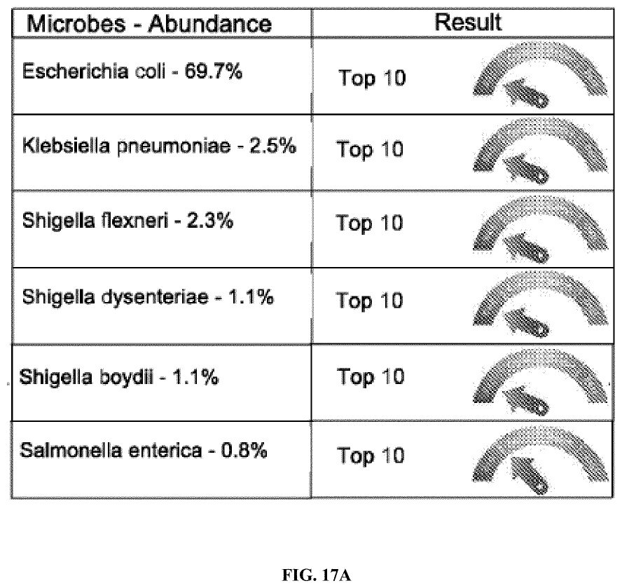

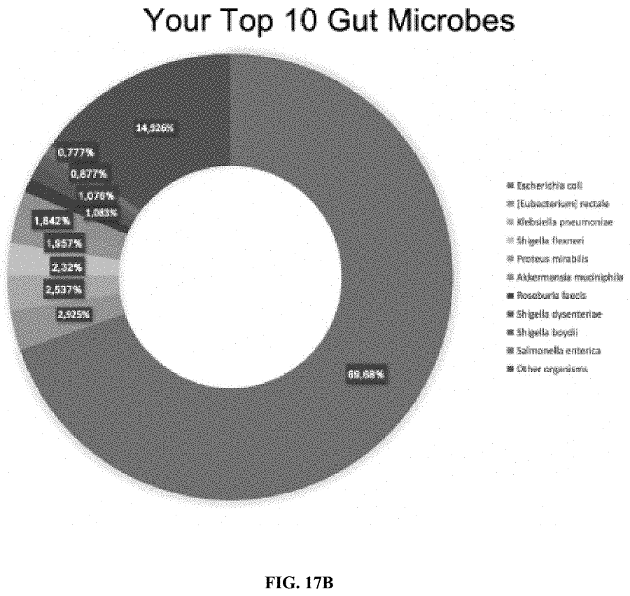

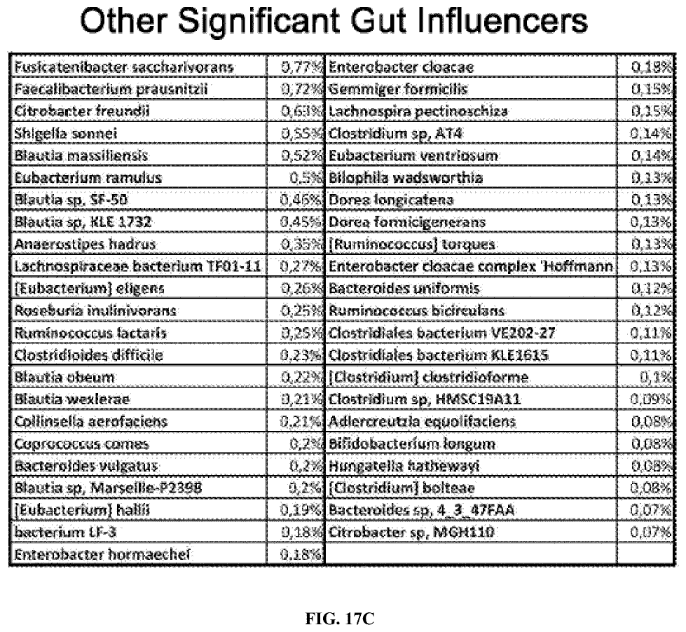

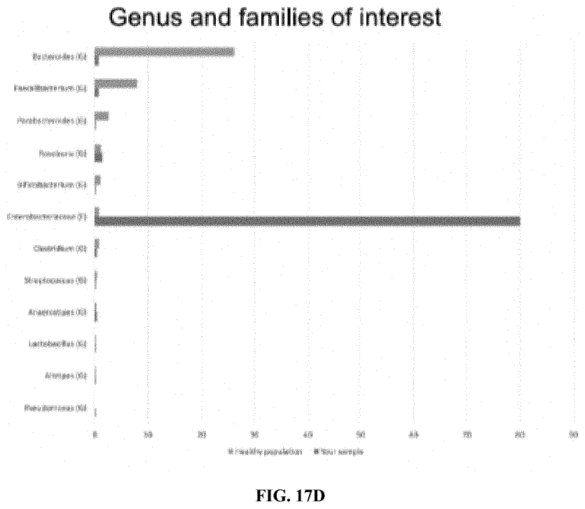

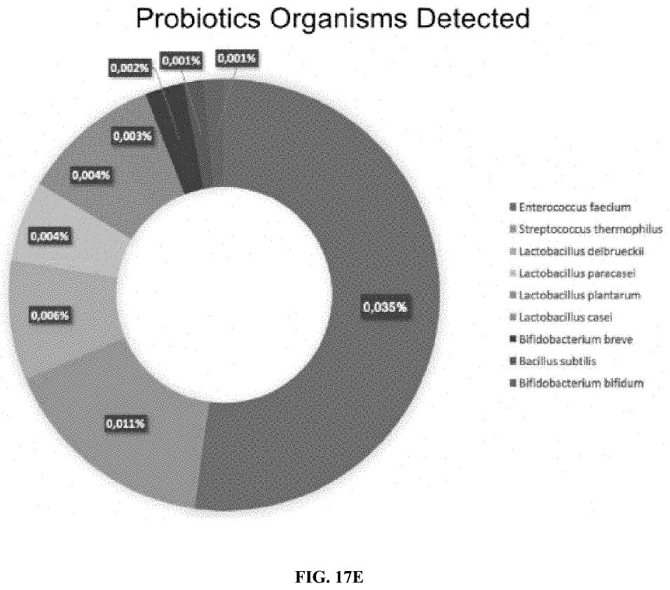

One subject's microbiome profile identified several gram negative microbes at the DNA level that may be associated with diarrhea or more complex gastrointestinal issues. The top ten microbe profile showed that E. coli made up almost 70% of the subject's microbiome compared with 40-60% of a healthy reference profile. This indicates that there was bacterial overgrowth (FIG. 17).

Using the disclosed methods specific probiotics combinations have been associated with specific microbiome profiles. Additionally, disease conditions have been identified that benefit from specific probiotic treatment (Table 5). For example Akkermansia may be used to metabolism and weight loss to treat obesity and chronic fatigue. Roseburia has been found useful for treating inflammatory bowel disease, irritable bowel syndrome, C. difficle infections or other gut inflammation issues, butyrate producing bacteria and possibly autoimmune issues.

Further, using the disclosed methods microbes impacting the microbiome and gut health have been identified, several of which had not previously been described (Table 6). For example, Bacteroides bulgatus and/or fragilis are associated with irritable bowel syndrome. In another example. Klebsiella, Eschicheria, Salmonella and/or Shingella may be used to diagnose cancer.

### Example 4

### Individual Microbiome Profiles

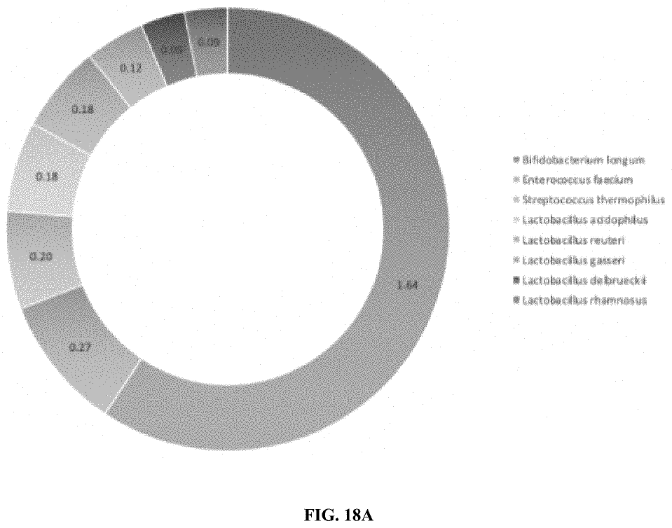

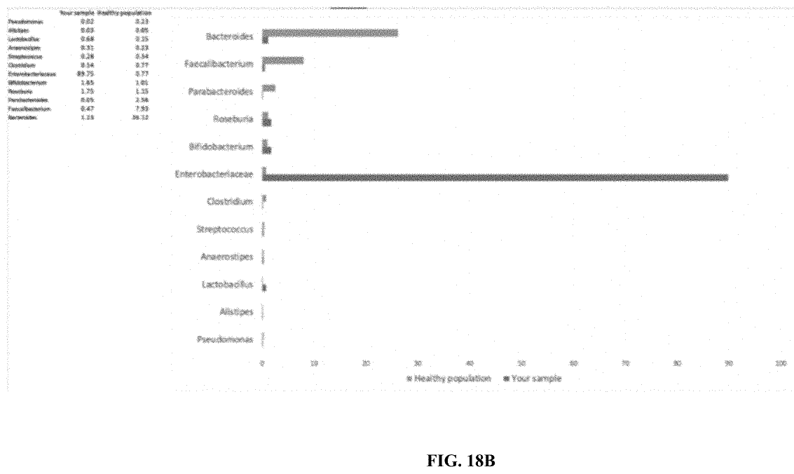

Using the methods previously described many subject microbiome profiles have been developed. Examples of the subject microbiome profiles are shown in FIGS. 18-24. In these examples, the top 10 microbes are identified and compared to the general population.

One example shows that subject E. coli made up 46.68% of the microbes with Klesiella pheumoniae being the next most prevalent microbe identified in the sample (FIG. 18A). Interestingly the analysis showed that the subject had significantly increased levels of Enterobacteriacae compared to the healthy reference (FIG. 18B).

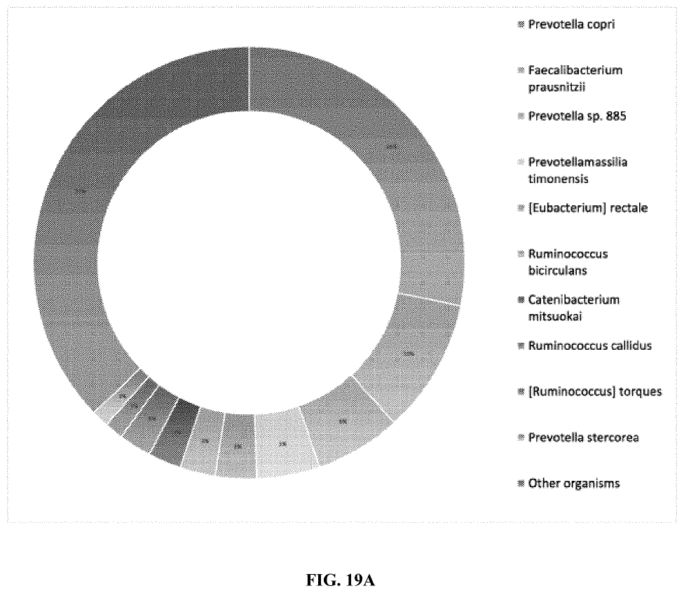

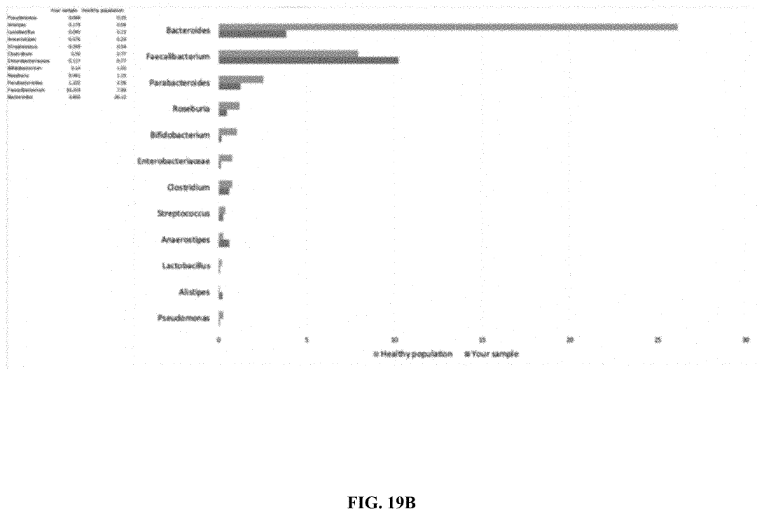

In another example, Prevotells copri was the most prevalent microbe followed by Faecalibacterium prausnitzii (FIG. 19A). This subject showed a significantly decreased level of Bacteroides compared with the healthy reference (FIG. 19B).

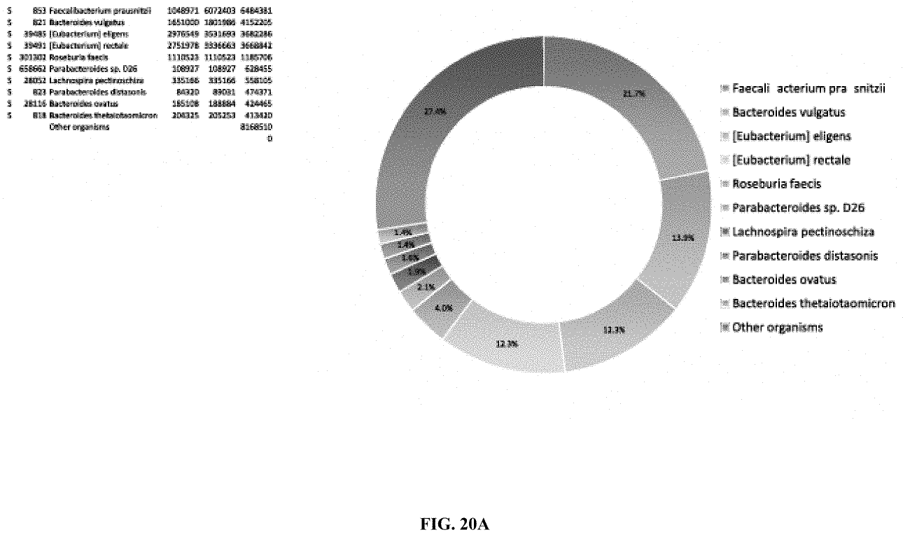

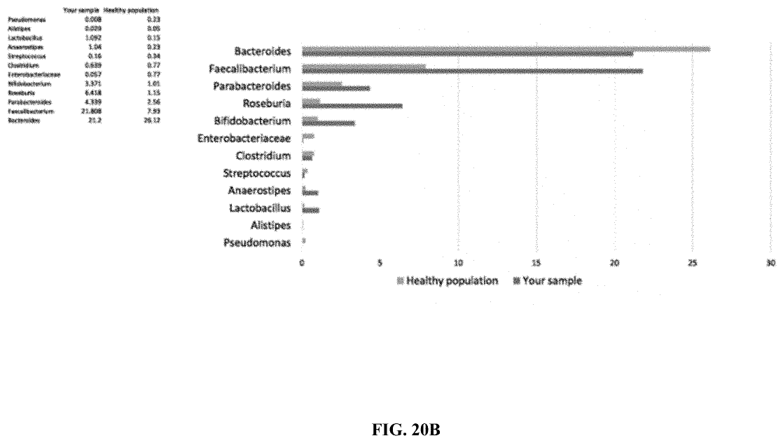

In an additional example, Faecalibacterium prausnitzii was the most prevalent microbe followed by Bacteroides vulgatus (FIG. 20A). This subject showed significantly increased levels of Faecalibacterium compared with the healthy reference (FIG. 20B).

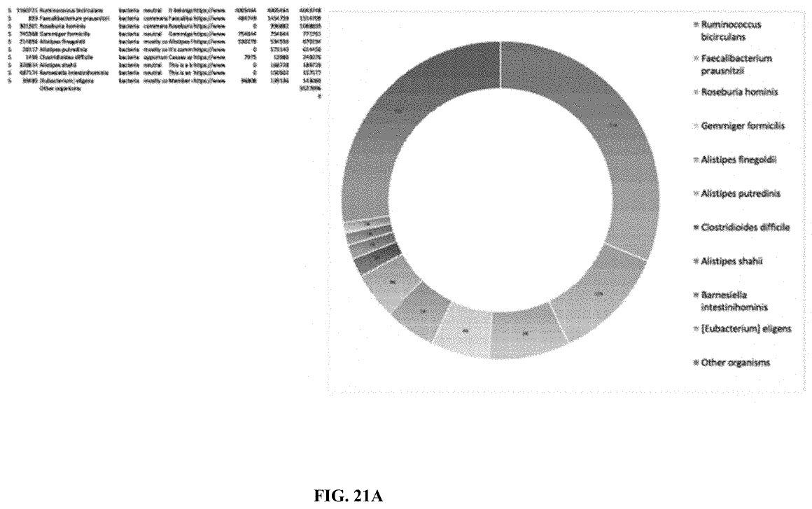

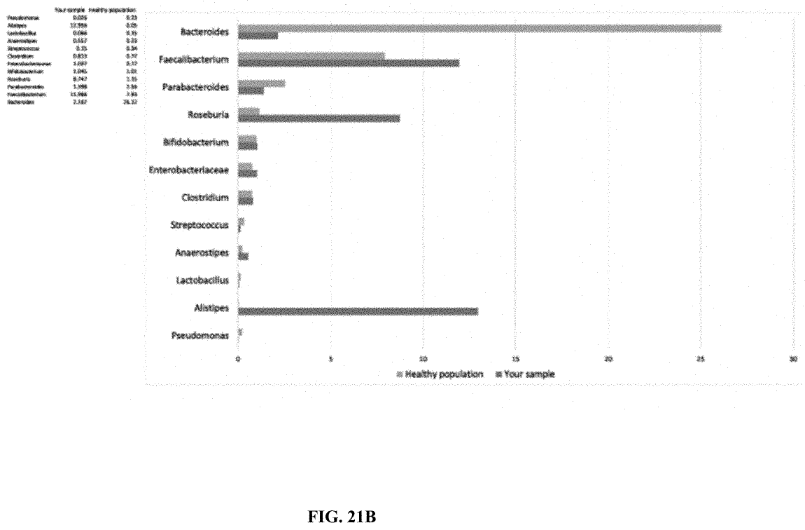

In a further example, Ruminococcus birculans was the most prevalent microbe followed by Faecalibacterium prausnitzii (FIG. 21A). This subject showed significantly increased levels of Bacteroides and Alistipes compared with the healthy reference (FIG. 21B).

In one example, Ruminococcus birculans was the most prevalent microbe followed by Faecalibacterium prausnitzii (FIG. 21A). This subject showed significantly increased levels of Bacteroides and Alistipes compared with the healthy reference (FIG. 21B).

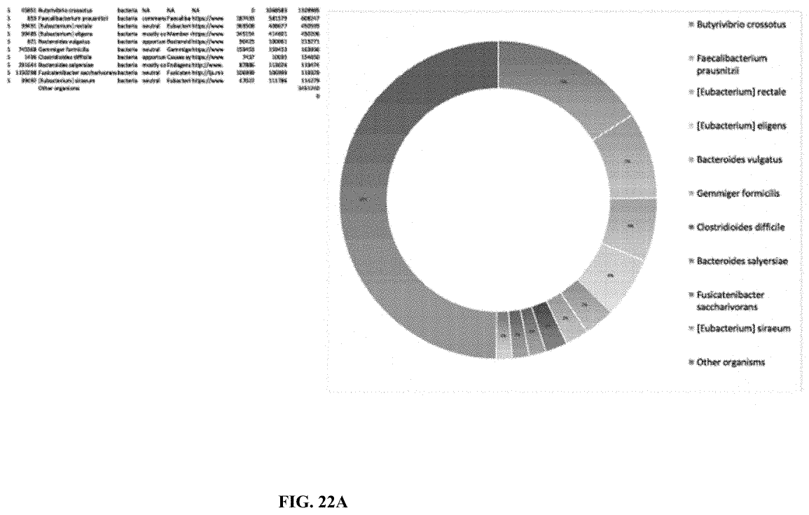

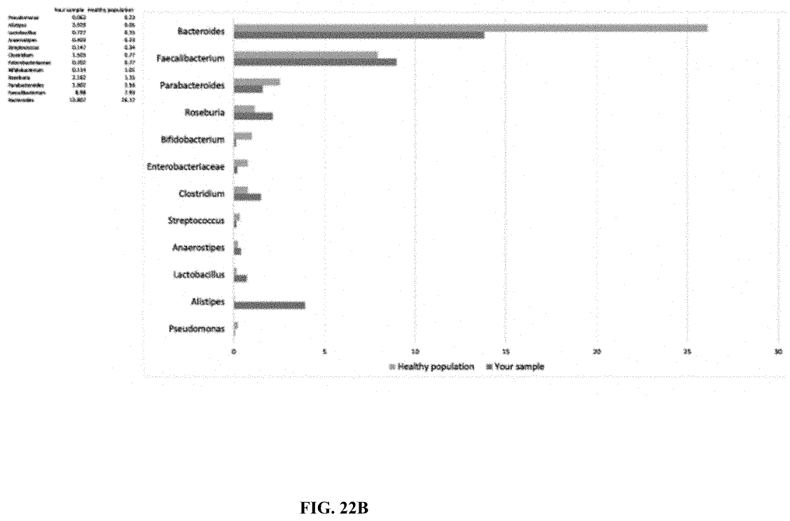

In one example, Butyrivibrio crossotus was the most prevalent microbe followed by Faecalibacterium prausnitzii (FIG. 22A). This subject showed significantly increased levels of Alistipes and significantly decreased levels of Bacteroides compared with the healthy reference (FIG. 22B).

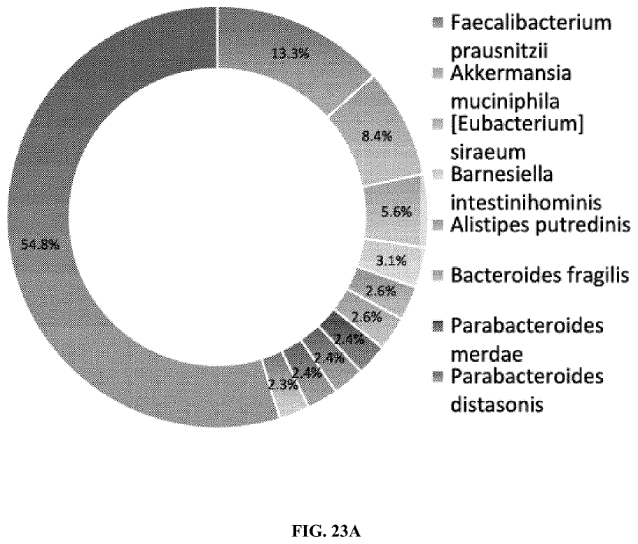

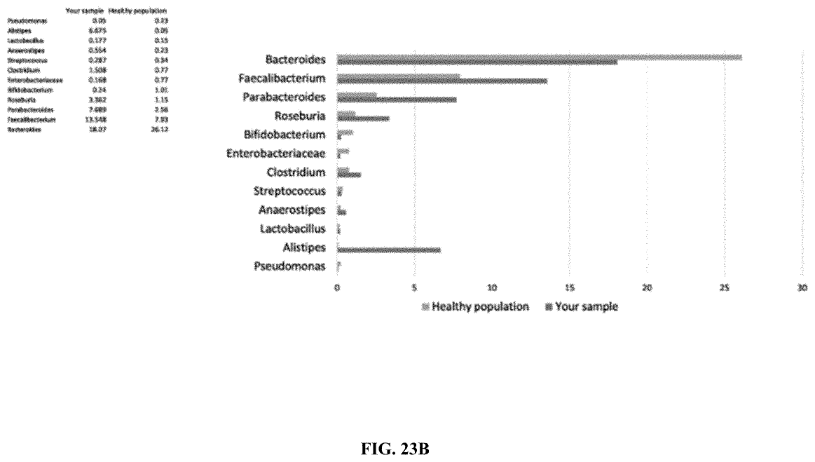

In one example, Faecalibacterium prausnitzii was the most prevalent microbe followed by Akkermansia muciniphila (FIG. 23A). This subject showed significantly increased levels of Alistipes, Faecalibacterium and Parabacteroides and significantly decreased levels of Bacteroides compared with the healthy reference (FIG. 23B).

Although the invention has been described with reference to the above example, it will be understood that modifications and variations are encompassed within the spirit and scope of the invention. Accordingly, the invention is limited only by the following claims.

1-73. (canceled)

74. A method of detecting, diagnosing and/or treating a subject comprising administering to the subject a formulation comprising a probiotic, pre-biotic, metabolite of the gut microbiome, or combination thereof, thereby causing a reduction or elimination of an opportunistic pathogen or a disease or disorder causing microbe of the gut.

75. The method of claim 74, further comprising detecting the opportunistic pathogen or the disease or disorder causing microbe of the gut.

76. The method of claim 75, further comprising isolating the opportunistic pathogen or the disease or disorder causing microbe of the gut.

77. The method of claim 75, wherein the opportunistic pathogen or the disease or disorder causing microbe of the gut is from a sample of the subject.

78. The method of claim 74, further comprising administering an isolated microbe from a sample obtained from the subject to the subject to reduce or eliminate the opportunistic pathogen or the disease or disorder causing microbe of the gut.

79. The method of claim 74, wherein the probiotic comprises a microbe isolated from a sample obtained from the subject.

80. The method of claim 74, further comprising diagnosing the disease or disorder in the subject by detecting a microbe of the gut obtained from the subject.

81. The method of claim 80, wherein the disease or disorder is selected from autism spectrum disorder, mood disorder, chronic fatigue, infection, necrosis, inflammation, autoimmune, hemorrhage, weight loss, metabolic disorder, irritable bowel disorder, diabetes 1 or 2, rheumatoid arthritis, cancer, and cardiovascular disorder.

82. The method of claim 81, wherein the disease or disorder is autism spectrum disorder.

83. The method of claim 74, wherein the disease or disorder is selected from those set forth in Tables 5 or 6.

84. The method of claim 80, further comprising administering a microbe to the subject to treat the disease or disorder, wherein the microbe is optionally isolated from a sample obtained from the subject.

85. The method of claim 84, wherein the disease or disorder is autism spectrum disorder.

86. The method of claim 74, wherein the probiotic comprises one or more microbes selected from those set forth in Tables 5-15.

87. The method of claim 86, wherein the one or more microbes is isolated from a gut sample obtained from the subject.

88. The method of claim 87, wherein the one or more microbes is selected from the group consisting of Clostridium bolteae, Bifidobacterium lactis, Lactobacillus acidophilus, Bifidobacterium longum, Bifidobacterium bifidum, Lactobacillus casei, Lactobacillus plantarum, Lactobacillus gasseri, Lactobacillus salivarius, Lactobacillus rhamnosus, Lactobacillus bulgaricus, Bacillus coagulans, and any combination thereof.

89. The method of claim 74, wherein presence of a Blastocystis genus organisms is reduced in the subject.

90. The method of claim 89, wherein the Blastocystis genus organism is B. hominis.

91. The method of claim 74, wherein presence of Clostridium bolteae, Toxoplasma gondii, Klebsiella pneumonia, Candidatus Methanomassillicoccus intestinalis or C. difficile is reduced in the subject.

92. The method of claim 87, wherein the one or more microbes is selected from the group consisting of Bacillus coagulans, Bacillus indicus, Bacillus lichenformis, Bacillus subtilis, Bifidobacterium animalis, Bifidobacterium bifidum, Bifidobacterium breve, Bifidobacterium coagilans, Bifidobacterium infantis, Bifidobacterium lactis, Bifidobacterium longum, Bifidobacterium subtilis, Enterococcus faecium, Lactobacillus acidophilus, Lactobacillus bulgaricus, Lactobacillus casei, Lactobacillus delbrueckii, Lactobacillus gasseri, Lactobacillus helveticus, Lactobacillus lactis, Lactobacillus paracasei, Lactobacillus plantarum, Lactobacillus reuteri, Lactobacillus rhamnosus, Lactobacillus salivarius, Saccharomyces boulardii, Streptococcus thermophiles, Lactobacillus buchneri, Lactobacillus fermentum, Lactobacillus crispatus, Bifidobacterium catenulatum, Bifidobacterium pseudocatenulatum, and combinations thereof.

93. The method of claim 84, wherein longevity and/or aging of the subject is improved.

94. The method of claim 74, further comprising confirming a shift and/or predicting a shift or a change in microflora of the subject using short, branched, or chemical fatty acid analysis of the subject.

95. The method of claim 94, wherein the fatty acid analysis includes detecting a marker selected from butyrate, propionate, acetate, or other fatty acid.

96. The method of claim 94, wherein the fatty acid analysis is used to determine differences between diagnosed autism spectrum disorder and normal phenotype.

97. A method of treating autism spectrum disorder in a subject comprising administering to the subject a probiotic including one or more microbes as set forth in Table 15 causing a reduction of Clostridium bolteae in the subject, thereby treating autism spectrum disorder in the subject.

98. The method of claim 97, wherein the one or more microbes is selected from the group consisting of Bifidobacterium lactis, Lactobacillus acidophilus, Bifidobacterium longum, Bifidobacterium bifidum, Lactobacillus casei, Lactobacillus plantarum, Lactobacillus gasseri, Lactobacillus salivarius, Lactobacillus rhamnosus, Lactobacillus bulgaricus, and Bacillus coagulans.
